(19) United States
(12) Patent Application Publication
(10) Pub. No.: US 2024/0246968 A1
(43) Pub. Date: Jul. 25, 2024

(54) SMALL MOLECULE INHIBITORS OF KRAS G12C MUTANT

(71) Applicant: Merck Sharp & Dohme LLC, Rahway, NJ (US)

(72) Inventors: Kazuaki SHIBATA, Ibaraki (JP); Hitomi KONDO, Ibaraki (JP); Tomohiro YAMAMOTO, Ibaraki (JP); Toshihiro SAKAMOTO, Ibaraki (JP); Hiroki ASAKURA, Ibaraki (JP); Kei AKEMOTO, Ibaraki (JP); Risako MIURA, Ibaraki (JP); Tetsuya SUGIMOTO, Ibaraki (JP); Juan DEL POZO, Brookline, MA (US); George Madalin GIAMBUSU, Natick, MA (US); Thomas H. GRAHAM, Somerville, MA (US); Yongxin HAN, Needham, MA (US); Elisabeth T. HENNESSY, Weston, MA (US); Shuhei KAWAMURA, Cambridge, MA (US); Michael RYAN, Brookline, MA (US)

(21) Appl. No.: 18/557,458

(22) PCT Filed: Apr. 27, 2022
(86) PCT No.: PCT/US2022/026601

§ 371 Date: Oct. 26, 2023

Related U.S. Application Data
(60) Provisional application No. 63/180,426, filed on Apr. 27, 2021.

(51) Int. Cl. C07D 471/04; A61K 31/437; A61K 31/4985; A61K 45/06; A61P 35/00; C07D 403/04; C07D 487/04; C07D 519/00

(52) U.S. Cl. CPC C07D 471/04; A61K 31/437; A61K 31/4985; A61K 45/06; A61P 35/00; C07D 403/04; C07D 487/04; C07D 519/00

(57) ABSTRACT

Compounds or their pharmaceutically acceptable salts can inhibit the G12C mutant of Kirsten rat sarcoma (KRAS) protein and are expected to have utility as therapeutic agents, for example, for treating cancer. The disclosure also provides pharmaceutical compositions which comprise compounds disclosed herein or pharmaceutically acceptable salts thereof. The disclosure also relates to methods for use of the compounds or their pharmaceutically acceptable salts in the therapy and prophylaxis of cancer and for preparing pharmaceuticals for this purpose.

### CROSS-REFERENCE TO RELATED APPLICATION

The present application claims the benefit of U.S. Provisional Application No. 63/180,426, filed Apr. 27, 2021, the entirety of which is incorporated by reference herein.

### BACKGROUND

The present disclosure relates to certain aniline compounds that inhibit the G12C mutant of Kirsten rat sarcoma (KRAS) protein and relates to a pharmaceutical composition comprising a compound disclosed herein as well as methods of using such a compound for treatment of diseases, including cancers.

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releasing GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts it to GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 95% of pancreatic cancers, in 45% of colorectal cancers, and in 35% of lung cancers. The mutations often occur in the glycine residue at position 12; in pulmonary adenocarcinoma, in particular, the mutation in the glycine residue at position 12 occurs in about 90% of the total KRAS mutations. Among such mutations, the most often occurring mutation (44%) has been reported to be a mutation into cysteine (Nature Reviews Drug Discovery, 13 (11), 828-51, 2014).

KRAS containing mutations are historically thought to exist in a constitutively active state (GTP-bound) in cancer cells. However, a recent study indicated that KRAS proteins having the G12C mutation have basal GTPase activity. K-Ras has a pocket structure to which a therapeutic agent can bind. Part of the pocket contains Switch 1 (residue 30 to 40) and Switch 2 (residue 60 to 76). Switch 1 has threonine-35 and Switch 2 has glycine-60, and these amino acids respectively form a hydrogen bond with the γ-phosphoric acid of GTP, which keeps Switch 1 and Switch 2 in an active form. These two regions will be released by hydrolysis of GTP and liberate phosphoric acid to form an inactive GDP form. When GTP bound to K-Ras is replaced with GDP, the three-dimensional conformation of the switch region containing these switches is changed. The change may relate to a bond between K-Ras and a target protein, such as c-Raf.

Actually, it was reported that ARS-853 binds to the cysteine residue of the G12C mutant of inactive KRAS (GDP), thus preventing conversion of inactive KRAS (GDP) to active KRAS (GTP), inhibiting downstream signaling, and inducing apoptosis in cancer cells with the KRAS G12C mutation (WO 2014/152588; Cancer Discov., 6 (3), 316-29, 2016). It has also been reported that ARS-1620 with a quinazoline backbone exerts antitumor action in tumor-bearing mice expressing the KRAS G12C mutation by improving metabolic stability in mice (WO 2015/054572; Cell, 172 (3), 578-89, 2018).

However, because of its mode of action, there is a possibility that the inhibitors which bind to the inactive form of KRAS G12C are not able to exert sufficient effect to KRAS G12C-positive cancer patients in whom the active form of KRAS protein (GTP) tends to be increased by activation of a KRAS upstream pathway or deactivation of GTPase activity in clinical settings. In fact, it has been reported that the inhibition of KRAS activity and the antiproliferative effect of ARS-853 are attenuated by EGFR activation in a KRAS G12C mutated cell line (Cancer Discov., 6 (3), 316-29, 2016; Science, 351 (6273), 604-8, 2016).

It is likely that the specific KRAS upstream inputs to KRAS protein or aberration of GAP function will be cancer-type- or patient-specific. Thus, appropriate treatment by GDP form KRAS G12C mutation inhibitors such as ARS-853 and ARS-1620 may require a precise understanding of tumor-specific signaling vulnerabilities upstream or downstream of the KRAS pathway. Because of that possible vulnerability, clinicians administering inhibitors for GDP form KRAS G12C mutation will need to select the effective patient population and likely have to choose the strategies of drug combination, based on the vulnerabilities of KRAS upstream/downstream signaling. On the other hand, inhibitors against the active form of KRAS G12C mutation are expected to provide therapeutic opportunities toward a wider population of G12C positive patients even as a single agent, with almost no effect from KRAS upstream status.

There is thus a need for a novel compound or a salt thereof that binds to the mutation. e.g., the mutant cysteine, of the active form of KRAS G12C mutation and a pharmaceutical composition comprising the same.

### SUMMARY OF THE DISCLOSURE

The present disclosure provides compounds or their pharmaceutically acceptable salts which modulate mutant KRAS protein and may be valuable pharmaceutically active compounds for the treatment of cancer. The compounds of the disclosure selectively inhibit the KRAS (G12C) protein. The disclosure furthermore provides processes for preparing compounds of the disclosure, methods for using such compounds to treat oncological disorders, and pharmaceutical compositions which comprise compounds of the disclosure.

### DETAILED DESCRIPTION

### Definitions

Unless defined otherwise, all technical and scientific terms used herein have the same meaning as is commonly understood by one of skill in the art to which this disclosure belongs.

As used throughout this disclosure, “compound(s) disclosed herein”, “compound(s) described herein”. “compound(s) of the disclosure”, etc., are used interchangeably and include both the compound, as well as a pharmaceutically acceptable salt thereof. Disclosure of a compound likewise discloses a pharmaceutically acceptable salt of the compound, and disclosure of a pharmaceutically acceptable salt of a compound likewise discloses the compound. i.e., the non-salt or un-ionized form of the compound, or an alternative pharmaceutically acceptable salt of the compound, as would be readily understood by one of ordinary skill in the art.

### Compounds of the Disclosure

In an embodiment, the compounds disclosed herein include:

| | | | | ESI-MS |
|---|---|---|---|---|
| Ex. | Structure | Name | NMR | [M + H]⁺ |
| 1 | 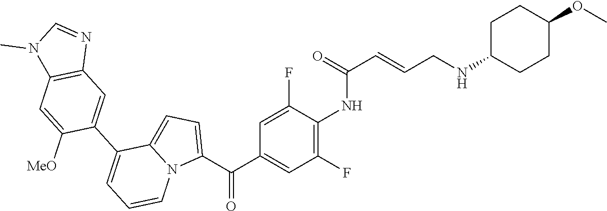 | (E)-N-(2,6-difluoro-4-(8-(6- methoxy-1-methyl-1H- benzo[d]imidazol-5- yl)indolizine-3- carbonyl)phenyl)-4-(((1r,4r)-4- methoxycyclohexyl)amino)but- 2-enamide | 1H-NMR (400 MHz, CHLOROFORM-d) δ 9.99 (d, J = 7.1 Hz, 1H), 7.87 (s, 1H), 7.78 (s, 1H), 7.50-7.39 (m, 2H), 7.34 (d, J = 4.9 Hz, 1H), 7.31- 7.26 (m, 1H), 7.19-7.05 (m, 2H), 6.98 (s, 1H), 6.34 (dd, J = 4.9, 0.6 Hz, 1H), 6.31-6.25 (m, 1H), 3.91 (s, 3H), 3.85 (s, 3H), 3.52 (dd, J = 5.3, 1.8 Hz, 2H), 3.37 (s, 3H), 3.22-3.13 (m, 1H), 2.61-2.48 (m, 1H), 2.15-2.07 (m, 2H), 2.00 (brd, J = 11.9 Hz, 2H), 1.31-1.12 (m, 4H) | 628.3 |
| 2 | 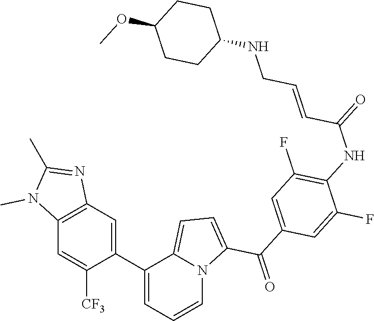 [SMILES: CO[C@H]1CC[C@H](NC/C=C/C(=O)Nc2c(F)cc(C(=O)c3ccc4c(-c5cc6nc(C)n(C)c6cc5C(F)(F)F)cccn34)cc2F)CC1] | (E)-N-(4-(8-(1,2-dimethyl-6- (trifluoromethyl)-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.99 (d, J = 7.0 Hz, 1H), 7.79 (s, 1H), 7.70 (s, 1H), 7.39 (br d, J = 4.1 Hz, 1H), 7.37-7.30 (m, 1H), 7.27-7.04 (m, 3H), 6.29 (br d, J = 15.4 Hz, 1H), 6.11 (d, J = 4.8 Hz, 1H), 3.88 (s, 3H), 3.63-3.44 (m, 2H), 3.36 (s, 3H), 3.25-3.07 (m, 1H), 2.71 (s, 3H), 2.56 (br t, J = 3.8 Hz, 1H), 2.15-1.96 (m, 4H), 1.38-1.12 (m, 4H) | 680.2 |
| 3 | 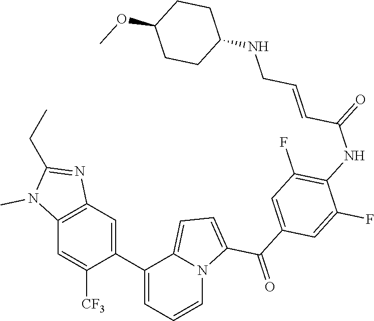 [SMILES: CCc1nc2cc(-c3cccn4c(C(=O)c5cc(F)c(NC(=O)/C=C/CN[C@H]6CC[C@H](OC)CC6)c(F)c5)ccc34)c(C(F)(F)F)cc2n1C] | (E)-N-(4-(8-(2-ethyl-1-methyl- 6-(trifluoromethyl)-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.98 (d, J = 7.0 Hz, 1H), 7.80 (s, 1H), 7.74 (s, 1H), 7.53-7.38 (m, 2H), 7.32 (d, J = 5.0 Hz, 1H), 7.21 (d, J = 6.9 Hz, 1H), 7.18-7.02 (m, 2H), 6.32 (br d, J = 15.4 H, 1H), 6.12 (d, J = 4.9 Hz, 1H), 3.88 (s, 3H), 3.54 (dd, J = 5.4, 1.6 Hz, 2H), 3.36 (s, 3H), 3.16 (br d, J = 4.0 Hz, 1H), 3.02 (q, J = 7.6 Hz, 2H), 2.60 (br d, J = 3.6 Hz, 1H), 2.14-1.93 (m, 4H), 1.35-1.16 (m, 4H) | 694.3 |
| 4 | 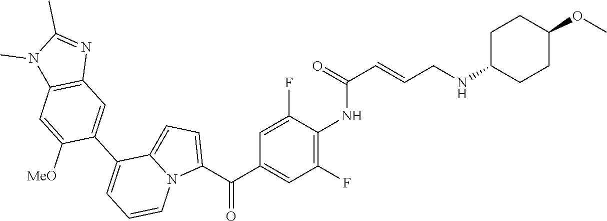 | (E)-N-(2,6-difluoro-4-(8-(6- methoxy-1,2-dimethyl-1H- benzo[d]imidazol-5- yl)indolizine-3- carbonyl)phenyl)-4-(((1r,4r)-4- methoxycyclohexyl)amino)but- 2-enamide | 1H-NMR (400 MHz, CHLOROFORM-d) δ 9.98 (d, J = 6.9 Hz, 1H), 7.66 (s, 1H), 7.46 (d, J = 8.7 Hz, 2H), 7.34 (d, J = 4.9 Hz, 1H), 7.17-7.08 (m, 2H), 6.96 (br s, 1H), 6.90 (s, 1H), 6.34 (dd, J = 4.8, 0.7 Hz, 1H), 6.25 (d, J = 15.4 Hz, 1H), 3.84 (s, 3H), 3.80 (s, 3H), 3.53 (dd, J = 5.3, 1.8 Hz, 2H), 3.38 (s, 3H), 3.21-3.13 (m, 1H), 2.65 (s, 3H), 2.60-2.52 (m, 1H), 2.14-2.08 (m, 2H), 2.05-1.99 (m, 2H), 1.25-1.10 (m, 4H) | 642.4 |
| 5 | 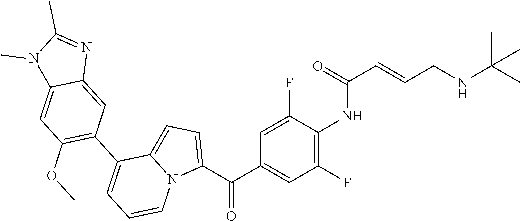 [SMILES: COc1cc2c(cc1-c1cccn3c(C(=O)c4cc(F)c(NC(=O)/C=C/CNC(C)(C)C)c(F)c4)ccc13)nc(C)n2C] | (E)-4-(tert-butylamino)-N-(2,6- difluoro-4-(8-(6-methoxy-1,2- dimethyl-1H-benzo[d]imidazol- 5-yl)indolizine-3- carbonyl)phenyl)but-2-enamide | 1H-NMR (400 MHz, CHLOROFORM-d) δ 9.96 (d, J = 6.9 Hz, 1H), 7.63 (s, 1H), 7.43-7.37 (m, 2H), 7.31 (d, J = 4.8 Hz, 1H), 7.29-7.22 (m, 2H), 7.19-7.01 (m, 2H), 6.87 (s, 1H), 6.35-6.31 (m, 1H), 6.31 (s, 1H), 3.81 (s, 3H), 3.77 (s, 3H), 3.45 (dd, J = 5.3, 1.8 Hz, 2H), 2.62 (s, 3H), 1.16 (s, 9H) | 586.4 |
| 6 | 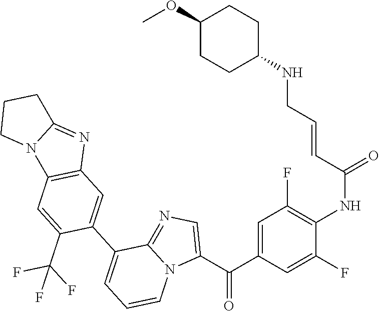 [SMILES: CO[C@H]1CC[C@H](NC/C=C/C(=O)Nc2c(F)cc(C(=O)c3cnc4c(-c5cc6nc7n(c6cc5C(F)(F)F)CCC7)cccn34)cc2F)CC1] | (E)-N-(2,6-difluoro-4-(8-(7- (trifluoromethyl)-2,3-dihydro- 1H-benzo[d]pyrrolo[1,2- a]imidazol-6-yl)imidazo[1,2- a]pyridine-3-carbonyl)phenyl)- 4-(((1r,4r)-4- methoxycyclohexyl)amino)but- 2-enamide | 1H NMR (400 MHz, CHLOROFORM-d) δ = 9.74 (br d, J = 6.8 Hz, 1H), 8.18 (s, 1H), 7.79 (s, 1H), 7.72 (s, 1H), 7.60-7.45 (m, 3H), 7.33-7.19 (m, 2H), 7.15-7.05 (m, 1H), 6.22 (br d, J = 15.3 Hz, 1H), 4.23 (br t, J = 6.8 Hz, 2H), 3.49 (br d, J = 4.0 Hz, 2H), 3.35 (s, 3H), 3.15 (br t, J = 7.1 Hz, 3H), 2.91-2.73 (m, 2H), 2.52 (br t, J = 10.1 Hz, 1H), 2.07 (br d, J = 10.4 Hz, 2H), 1.97 (br d, J = 10.9 Hz, 2H), 1.34-1.07 (m, 4H) | 693.3 |
| 7 | 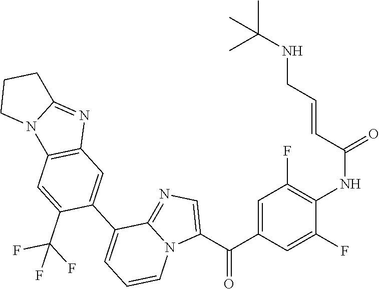 [SMILES: CC(C)(C)NC/C=C/C(=O)Nc1c(F)cc(C(=O)c2cnc3c(-c4cc5nc6n(c5cc4C(F)(F)F)CCC6)cccn23)cc1F] | (E)-4-(tert-butylamino)-N-(2,6- difluoro-4-(8-(7- (trifluoromethyl)-2,3-dihydro- 1H-benzo[d]pyrrolo[1,2- a]imidazol-6-yl)imidazo[1,2- a]pyridine-3- carbonyl)phenyl)but-2-enamide | 1H NMR (400 MHz, CHLOROFORM-d) δ = 9.75 (dd, J = 6.9, 1.2 Hz, 1H), 8.18 (s, 1H), 7.80 (s, 1H), 7.73 (s, 1H), 7.57-7.46 (m, 3H), 7.29- 7.23 (m, 1H), 7.19-7.08 (m, 2H), 6.25 (dt, J = 15.3, 1.8 Hz, 1H), 4.23 (t, J = 7.1 Hz, 2H), 3.44 (dd, J = 1.8, 5.0 Hz, 2H), 3.15 (t, J = 7.6 Hz, 2H), 2.88-2.76 (m, 2H), 1.14 (s, 9H) | 637.3 |
| 8 | 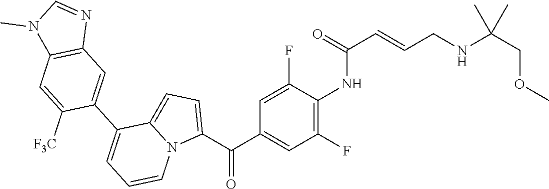 | (E)-N-(2,6-difluoro-4-(8-(1- methyl-6-(trifluoromethyl)-1H- benzo[d]imidazol-5- yl)indolizine-3- carbonyl)phenyl)-4-((1- methoxy-2-methylpropan-2- yl)amino)but-2-enamide | 1H-NMR (400 MHz, CHLOROFORM-d) δ 9.97 (d, J = 6.9 Hz, 1H), 8.10 (s, 1H), 7.91 (s, 1H), 7.83 (s, 1H), 7.44 (s, 1H), 7.42 (s, 1H), 7.32 (d, J = 4.8 Hz, 1H), 7.24-7.10 (m, 3H), 7.07 (t, J = 7.0 Hz, 1H), 6.32-6.19 (m, 2H), 6.10 (d, J = 4.8 Hz, 1H), 6.04 (d, J = 7.1 Hz, 1H), 4.00 (s, 3H), 3.40 (dd, J = 1.8, 5.1 Hz, 2H), 3.39-3.35 (m, 5H), 3.33 (s, 1H, 3.21 (s, 2H), 1.09 (s, 6H) | 640.3 |
| 9 | 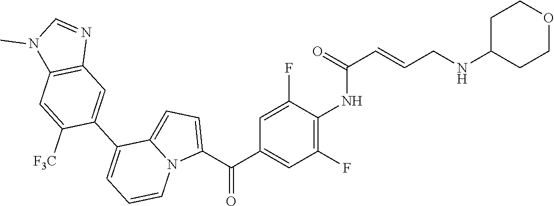 [SMILES: Cn1cnc2cc(-c3cccn4c(C(=O)c5cc(F)c(NC(=O)/C=C/CNC6CCOCC6)c(F)c5)ccc34)c(C(F)(F)F)cc21] | (E)-N-(2,6-difluoro-4-(8-(1- methyl-6-(trifluoromethyl)-1H- benzo[d]imidazol-5- yl)indolizine-3- carbonyl)phenyl)-4- ((tetrahydro-2H-pyran-4- yl)amino)but-2-enamide | 1H-NMR (400 MHz, CHLOROFORM-d) δ 9.98 (d, J = 6.9 Hz, 1H), 8.09 (s, 1H), 7.91 (s, 1H), 7.83 (s, 1H), 7.45 (s, 1H), 7.43 (s, 1H), 7.32 (d, J = 4.8 Hz, 1H), 7.24-7.17 (m, 1H), 7.17-7.00 (m, 3H), 6.32-6.22 (m, 1H), 6.11 (d, J = 4.8 Hz, 1H), 4.00 (s, 3H), 4.03-3.96 (m, 1H), 3.53 (dd, J = 5.2, 1.7 Hz, 2H), 3.42 (td, J = 11.7, 2.1 Hz, 2H), 2.80-2.62 (m, 1H), 1.93-1.81 (m, 2H), 1.49- 1.36 (m, 2H) | 638.3 |
| 10 | 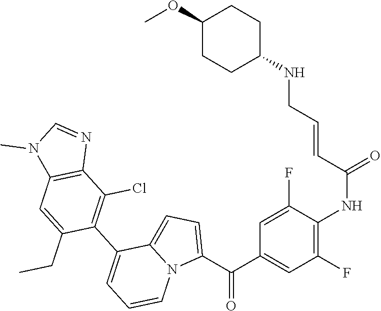 [SMILES: CCc1cc2c(ncn2C)c(Cl)c1-c1cccn2c(C(=O)c3cc(F)c(NC(=O)/C=C/CN[C@H]4CC[C@H](OC)CC4)c(F)c3)ccc12] | (E)-N-(4-(8-(4-chloro-6-ethyl- 1-methyl-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 10.00 (d, J = 6.8 Hz, 1H), 7.95 (s, 1H), 7.44 (d, J = 8.0 Hz, 2H), 7.32 (d, J = 5.3 Hz, 2H), 7.18-7.08 (m, 4H), 6.26 (d, J = 15.5 Hz, 1H), 6.05 (d, J = 4.8 Hz, 1H), 3.92 (s, 3H), 3.51 (d, J = 5.3 Hz, 2H), 3.35 (s, 3H), 3.17-3.12 (m, 1H), 2.56-2.44 (m, 3H), 2.09-1.97 (m, 4H), 1.30-1.16 (m, 4H), 1.09 (t, J = 7.5 Hz, 3H) | 660.0 |
| 11 | 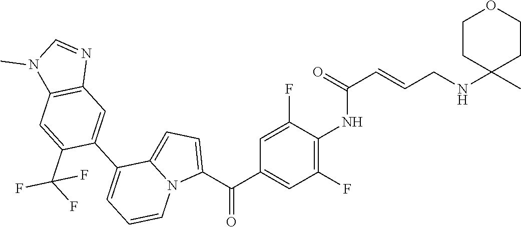 [SMILES: Cn1cnc2cc(-c3cccn4c(C(=O)c5cc(F)c(NC(=O)/C=C/CNC6(C)CCOCC6)c(F)c5)ccc34)c(C(F)(F)F)cc21] | (E)-N-(2,6-difluoro-4-(8-(1- methyl-6-(trifluoromethyl)-1H- benzo[d]imidazol-5- yl)indolizine-3- carbonyl)phenyl)-4-((4- methyltetrahydro-2H-pyran-4- yl)amino)but-2-enamide | 1H-NMR (400 MHz, CHLOROFORM-d) δ 9.98 (d, J = 7.0 Hz, 1H), 8.10 (s, 1H), 7.91 (s, 1H), 7.83 (s, 1H), 7.47-7.37 (m, 2H), 7.32 (d, J = 4.9 Hz, 1H), 7.24-7.06 (m, 3H), 6.35 (dd, J = 15.3, 1.6 Hz, 1H), 6.11 (d, J = 4.8 Hz, 1H), 4.00 (s, 3H), 3.88-3.77 (m, 2H), 3.67-3.56 (m, 2H), 3.46-3.39 (m, 2H), 1.62-1.54 (m, 4H), 1.17 (s, 3H) | 652.4 |
| 12-1, 12-2 | 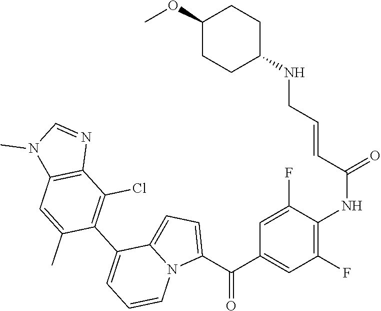 [SMILES: CO[C@H]1CC[C@H](NC/C=C/C(=O)Nc2c(F)cc(C(=O)c3ccc4c(-c5c(C)cc6c(ncn6C)c5Cl)cccn34)cc2F)CC1] | (E)-N-(4-(8-(4-chloro-1,6- dimethyl-1H-benzo[d]imidazol- 5-yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 10.01 (d, J = 6.6 Hz, 1H), 7.96 (s, 1H), 8.01-7.90 (m, 1H), 7.55-7.43 (m, 2H), 7.39-7.30 (m, 3H), 7.27- 7.10 (m, 3H), 6.97 (s, 1H), 6.25 (br d, J = 15.3 Hz, 1H), 6.09 (d, J = 4.8 Hz, 1H), 3.92 (s, 3H), 3.53 (dd, J = 5.1, 1.4 Hz, 2H), 3.37 (s, 3H), 3.21- 3.13 (m, 1H), 2.62-2.51 (m, 1H), 2.23 (s, 3H), 2.15-1.96 (m, 4H), 1.3-1.11 (m, 4H) | 646.4 |
| 13-1, 13-2 | 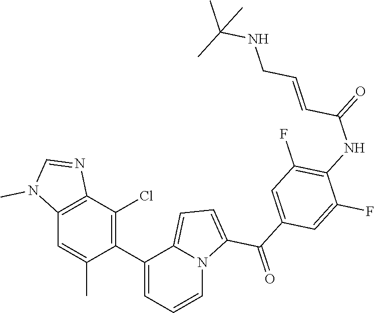 [SMILES: Cc1cc2c(ncn2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-1,6-dimethyl-1H- benzo[d]imidazol-5- yl)indolizine-3-carbonyl)-2,6- difluorophenyl)but-2-enamide (1^{st} eluting isomer and 2^{nd} eluting isomer) | 1H-NMR (400 MHz, CHLOROFORM-d) δ 9.99 (dd, J = 6.8, 0.8 Hz, 1H), 7.94 (s, 1H), 7.45 (d, J = 7.8 Hz, 2H), 7.33 (d, J = 4.8 Hz, 1H), 7.29 (d, J = 0.8 Hz, 1H), 7.17-7.11 (m, 3H), 6.29 (d, J = 15.5 Hz, 1H), 6.06 (dd, J = 4.9, 0.6 Hz, 1H), 3.90 (s, 3H), 3.46 (dd, J = 5.1, 1.9 Hz, 2H), 2.20 (d, J = 0.8 Hz, 3H), 1.16 (s, 9H) | 590.3 |
| 14-1, 14-2 | 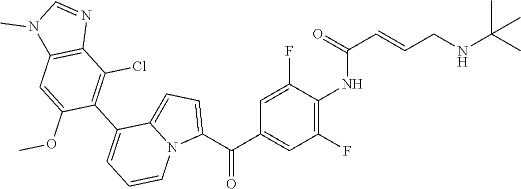 [SMILES: COc1cc2c(ncn2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-6-methoxy-1- methyl-1H-benzo[d]imidazol- 5-yl)indolizine-3-carbonyl)-2,6- difluorophenyl)but-2-enamide (1^{st} eluting isomer and 2^{nd} eluting isomer) | 1H-NMR (400 MHz, CHLOROFORM-d) 9.99 (d, J = 7.0 Hz, 1H), 7.89 (s, 1H), 7.43 (d, J = 7.9 Hz, 2H), 7.31 (d, J = 4.8 Hz, 1H), 7.20 (dd, J = 7.0, 1.1 Hz, 1H), 7.15 (dt, J = 15.4, 5.2 Hz, 1H), 7.11 (t, J = 7.0 Hz, 1H), 6.88 (s, 1H), 6.30 (d, J = 15.4 Hz, 1H), 6.10 (dd, J = 4.8, 0.6 Hz, 1H), 3.90 (s, 3H), 3.77 (s, 3H), 3.46 (dd, J = 5.2, 1.8 Hz, 1H), 1.16 (s, 9H) | 606.5 |
| 15-1, 15-2 | 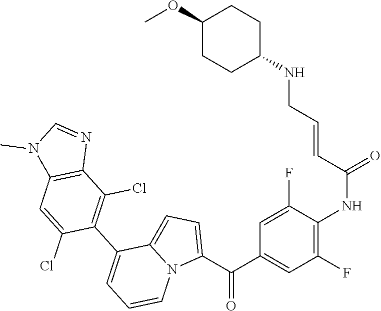 [SMILES: CO[C@H]1CC[C@H](NC/C=C/C(=O)Nc2c(F)cc(C(=O)c3ccc4c(-c5c(Cl)cc6c(ncn6C)c5Cl)cccn34)cc2F)CC1] | (E)-N-(4-(8-(4,6-dichloro-1- methyl-1H-benzo[d]imidazol- 5-yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 10.01 (d, J = 6.8 Hz, 1H), 8.01 (s, 1H), 7.56 (s, 1H), 7.48-7.44 (m, 2H), 7.36 (d, J = 4.8 Hz, 1H), 7.21 (dd, J = 6.9, 1.1 Hz, 1H), 7.15-7.08 (m, 2H), 6.25 (dd, J = 13.8, 1.8 Hz, 1H), 6.10 (d, J = 4.8 Hz, 1H), 3.92 (s, 3H), 3.51 (dd, J = 5.3, 1.8 Hz, 2H), 3.35 (s, 3H), 3.17-3.14 (m, 1H), 2.55-2.51 (m, 1H), 2.09-1.97 (m, 4H), 1.26-1.13 (m, 4H) | 666.4 |
| 16 | 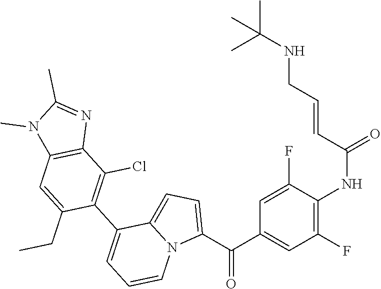 [SMILES: CCc1cc2c(nc(C)n2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-6-ethyl-1,2- dimethyl-1H-benzo[d]imidazol- 5-yl)indolizine-3-carbonyl)-2,6- difluorophenyl)but-2-enamide | 1H-NMR (400 MHz, CHLOROFORM-d) δ 10.00 (d, J = 6.8 Hz, 1H), 7.48-7.42 (m, 2H), 7.33 (d, J = 4.8 Hz, 1H), 7.27-7.01 (m, 5H), 6.30 (dd, J = 15.4, 1.6 Hz, 1H), 6.06 (d, J = 4.9 Hz, 1H), 3.81 (s, 3H), 3.52-3.38 (m, 2H), 2.69 (s, 3H), 2.59- 2.41 (m, 2H), 1.17 (s, 9H), 1.09 (t, J = 7.5 Hz, 3H) | 618.4 |
| 17 | 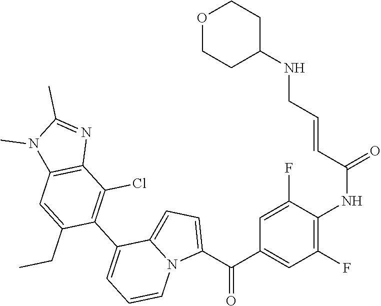 [SMILES: CCc1cc2c(nc(C)n2C)c(Cl)c1-c1cccn2c(C(=O)c3cc(F)c(NC(=O)/C=C/CNC4CCOCC4)c(F)c3)ccc12] | (E)-N-(4-(8-(4-chloro-6-ethyl- 1,2-dimethyl-1H- benzo[d]imidazol-5- yl)indolizine-3-carbonyl)-2,6- difluorophenyl)-4-((tetrahydro- 2H-pyran-4-yl)amino)but-2- enamide | 1H-NMR (400 MHz, CHLOROFORM-d) δ 10.01 (d, J = 6.9 Hz, 1H), 7.47 (br d, J = 8.1 Hz, 2H), 7.33 (d, J = 4.9 Hz, 1H), 7.27-7.08 (m, 4H), 6.29 (br d, J = 15.4 Hz, 1H), 6.07 (d, J = 4.8 Hz, 1H), 4.05-3.98 (m, 2H), 3.82 (s, 3H), 3.55 (dd, J = 5.1, 1.5 Hz, 2H), 3.44 (dt, J = 11.6, 1.8 Hz, 2H), 2.78 (tt, J = 10.5, 4.0 Hz, 1H), 2.70 (s, 3H), 2.60-2.42 (m, 2H), 1.93-1.84 (m, 2H), 1.57-1.40 (m, 2H), 1.10 (t, J = 7.6 Hz, 3H) | 646.3 |
| 18-1, 18-2 | 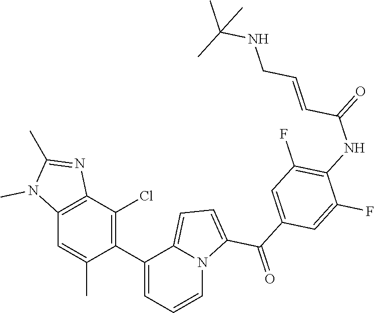 [SMILES: Cc1cc2c(nc(C)n2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-1,2,6-trimethyl- 1H-benzo[d]imidazol-5- yl)indolizine-3-carbonyl)-2,6- difluorophenyl)but-2-enamide (1^{st} eluting isomer and 2^{nd} eluting isomer) | 1H-NMR (400 MHz, CHLOROFORM-d) δ 9.98 (d, J = 6.8 Hz, 1H), 7.46-7.42 (m, 2H), 7.32 (d, J = 4.8 Hz, 1H), 7.17-7.10 (m, 4H), 6.29 (dt, J = 15.3, 1.8 Hz, 1H), 6.05 (d, J = 5.0 Hz, 1H), 3.78 (s, 3H), 3.45 (dd, J = 5.1, 1.9 Hz, 2H), 2.67 (s, 3H), 2.18 (s, 3H), 1.15 (s, 9H) | 604.4 |
| 19-1, 19-2 | 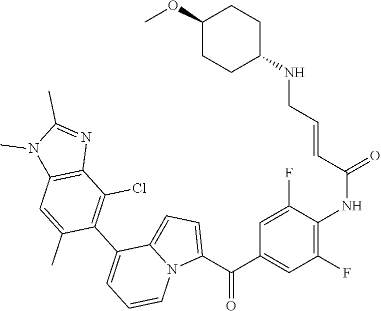 [SMILES: CO[C@H]1CC[C@H](NC/C=C/C(=O)Nc2c(F)cc(C(=O)c3ccc4c(-c5c(C)cc6c(nc(C)n6C)c5Cl)cccn34)cc2F)CC1] | (E)-N-(4-(8-(4-chloro-1,2,6- trimethyl-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.98 (dd, J = 6.8, 1.0 Hz, 1 H), 7.47-7.43 (m, 2H), 7.32 (d, J = 4.8 Hz, 1H), 7.17-7.08 (m, 4H), 6.24 (dt, J = 15.3, 1.6 Hz, 1H), 6.06 (dd, J = 4.8, 0.5 Hz, 1H), 3.78 (s, 3H), 3.51 (dd, J = 5.3, 2.0 Hz, 2H), 3.35 (s, 3H), 3.17-3.12 (m, 1H), 2.67 (s, 3H), 2.56-2.51 (m, 1H), 2.18 (s, 3H), 2.09-1.97 (m, 4H), 1.31-1.13 (m, 4H) | 660.4 |
| 20-1, 20-2 | 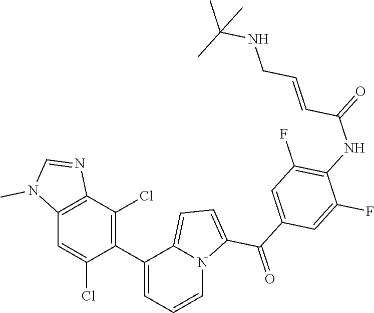 [SMILES: Cn1cnc2c(Cl)c(-c3cccn4c(C(=O)c5cc(F)c(NC(=O)/C=C/CNC(C)(C)C)c(F)c5)ccc34)c(Cl)cc21] | (E)-4-(tert-butylamino)-N-(4- (8-(4,6-dichloro-1-methyl-1H- benzo[d]imidazol-5- yl)indolizine-3-carbonyl)-2,6- difluorophenyl)but-2-enamide (1^{st} eluting isomer and 2^{nd} eluting isomer) | 1H-NMR (400 MHz, CHLOROFORM-d) δ 10.01 (d, J = 7.0 Hz, 1H), 8.01 (s, 1H), 7.56 (s, 1H), 7.46 (dd, J = 10.6, 2.9 Hz, 2H), 7.36 (d, J = 4.8 Hz, 1H), 7.21 (dd, J = 6.9, 1.1 Hz, 1H), 7.17-7.11 (m, 2H), 6.31-6.27 (m, 1H), 6.10 (d, J = 5.0 Hz, 1H), 3.92 (s, 3H), 3.46 (dd, J = 5.3, 1.8 Hz, 2H), 1.16 (s, 9H) | 610.3 |
| 21 | 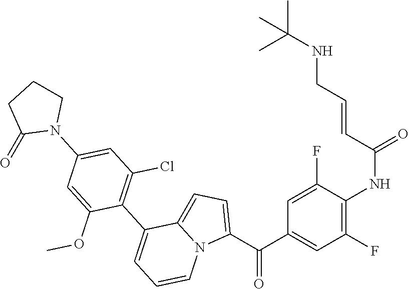 [SMILES: COc1cc(N2CCCC2=O)cc(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(2-chloro-6-methoxy-4-(2- oxopyrrolidin-1- yl)phenyl)indolizine-3- carbonyl)-2,6- difluorophenyl)but-2-enamide | 1H-NMR (400 MHz, CHLOROFORM-d) δ 9.97 (d, J = 6.9 Hz, 1H), 7.77 (d, J = 1.9 Hz, 1H), 7.40 (d, J = 7.9 Hz, 1H), 7.33 (d, J = 4.8 Hz, 1H), 7.17-7.06 (m, 4H), 6.32 (d, J = 15.5 Hz, 1H), 6.15 (d, J = 4.8 Hz, 1H), 3.92 (t, J = 7.0 Hz, 2H), 3.72 (s, 3H), 3.45 (dd, J = 5.3, 1.8 Hz, 2H), 2.68 (t, J = 8.1 Hz, 2H), 2.23 (t, J = 7.6 Hz, 2H), 1.15 (s, 9H) | 635.5 |
| 22-1, 22-2 | 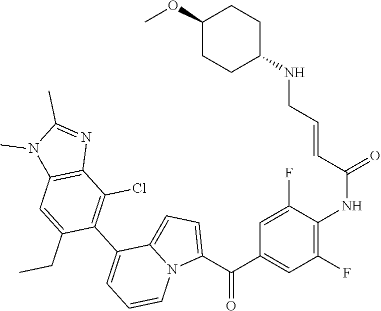 [SMILES: CCc1cc2c(nc(C)n2C)c(Cl)c1-c1cccn2c(C(=O)c3cc(F)c(NC(=O)/C=C/CN[C@H]4CC[C@H](OC)CC4)c(F)c3)ccc12] | (E)-N-(4-(8-(4-chloro-6-ethyl- 1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 10.00 (d, J = 6.7 Hz, 1H), 7.55-7.47 (m, 1H), 7.47-7.36 (m, 2H), 7.32 (d, J = 4.9 Hz, 1H), 7.27-7.24 (m, 1H), 7.24-7.16 (m, 2H), 7.16-7.01 (m, 2H), 6.30 (d, J = 15.4 Hz, 1H), 6.06 (dd, J = 4.8, 0.7 Hz, 1H), 3.81 (s, 3H), 3.56-3.44 (m, 2H), 3.36 (s, 3H), 3.23-3.08 (m, 1H), 2.68 (s, 3H), 2.60-2.40 (m, 3H), 2.16-1.90 (m, 4H), 1.87-1.68 (m, 2H), 1.34-1.12 (m, 4H), 1.08 (t, J = 7.5 Hz, 3H) | 674.4 |
| 23 | 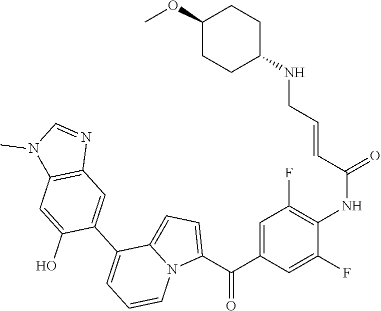 [SMILES: CO[C@H]1CC[C@H](NC/C=C/C(=O)Nc2c(F)cc(C(=O)c3ccc4c(-c5cc6ncn(C)c6cc5O)cccn34)cc2F)CC1] | (E)-N-(2,6-difluoro-4-(8-(6- hydroxy-1-methyl-1H- benzo[d]imidazol-5- yl)indolizine-3- carbonyl)phenyl)-4-(((1r,4r)-4- methoxycyclohexyl)amino)but- 2-enamide | 1H-NMR (400 MHz, DMSO-d6) δ 10.00 (s, 1H), 9.86 (d, J = 7.0 Hz, 1H), 9.57 (s, 1H), 8.06 (s, 1H), 7.57-7.50 (m, 3H), 7.42 (d, J = 4.8 Hz, 1H), 7.34 (dd, J = 7.0, 1.0 Hz, 1H), 7.25 (dd, J = 7.0, 7.0 Hz, 1H), 7.03 (s, 1H), 6.87 (dt, J = 15.4, 5.1 Hz, 1H), 6.38-6.31 (m, 2H), 3.79 (s, 3H), 3.39- 3.36 (m, 2H), 3.22 (s, 3H), 3.12-3.04 (m, 1H), 2.40 (s, 1H), 2.01-1.84 (m, 4H), 1.18-0.98 (m, 4H) | 614.6 |
| 24 | 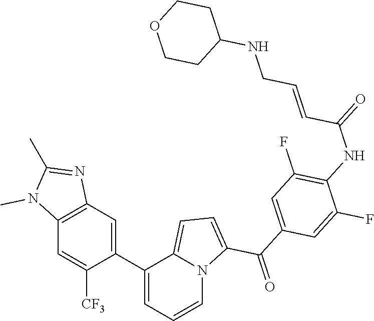 [SMILES: Cc1nc2cc(-c3cccn4c(C(=O)c5cc(F)c(NC(=O)/C=C/CNC6CCOCC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((tetrahydro- 2H-pyran-4-yl)amino)but-2- enamide | 1H-NMR (400 MHz, CHLOROFORM-d) δ 9.97 (d, J = 7.0 Hz, 1H), 7.78 (s, 1H), 7.68 (s, 1H), 7.45-7.41 (m, 2H), 7.31 (d, J = 4.8 Hz, 1H), 7.20 (d, J = 7.0 Hz, 1H), 7.15-7.06 (m, 2H), 6.28 (dd, J = 13.6, 1.6 Hz, 1H), 6.10 (dd, J = 4.9, 0.6 Hz, 1H), 4.01-3.97 (m, 2H), 3.87 (s, 3H), 3.53 (dd, J = 5.3, 1.8 Hz, 2H), 3.41 (td, J = 11.6,2.1 Hz, 2H), 2.79-2.74 (m, 1H), 2.70 (s, 3H), 1.88-1.84 (m, 2H), 1.48-1.39 (m, 2H) | 652.5 |
| 25 | 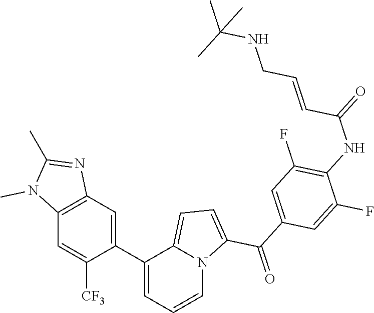 [SMILES: Cc1nc2cc(-c3cccn4c(C(=O)c5cc(F)c(NC(=O)/C=C/CNC(C)(C)C)c(F)c5)ccc34)c(C(F)(F)F)cc2n1C] | (E)-4-(tert-butylamino)-N-(4- (8-(1,2-dimethyl-6- (trifluoromethyl)-1H- benzo[d]imidazol-5- yl)indolizine-3-carbonyl)-2,6- difluorophenyl)but-2-enamide | 1H-NMR (400 MHz, CHLOROFORM-d) δ 9.97 (d, J = 7.3 Hz, 1H), 7.78 (s, 1H), 7.68 (s, 1H), 7.45-7.41 (m, 2H), 7.30 (t, J = 4.8 Hz, 1H), 7.20- 7.05 (m, 3H), 6.31 (d, J = 15.3 Hz, 1H), 6.10 (d, J = 5.0 Hz, 1H), 3.86 (s, 3H), 3.46 (dd, J = 5.3, 1.5 Hz, 2H), 2.70 (s, 3H), 1.17 (s, 9H) | 624.5 |
| 26 | 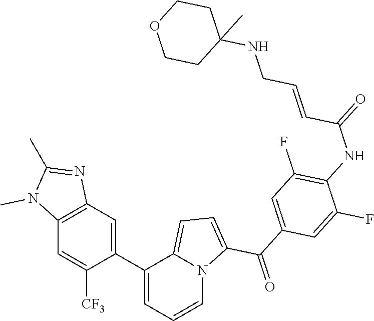 [SMILES: Cc1nc2cc(-c3cccn4c(C(=O)c5cc(F)c(NC(=O)/C=C/CNC6(C)CCOCC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((4- methyltetrahydro-2H-pyran-4- yl)amino)but-2-enamide | 1H-NMR (400 MHz, CHLOROFORM-d) δ 9.97 (dd, J = 7.1, 0.9 Hz, 1H), 7.78 (s, 1H), 7.68 (s, 1H), 7.42-7.38 (m, 2H), 7.30 (d, J = 5.0 Hz, 1H), 7.20 (d, J = 7.0 Hz, 1H), 7.13 (td, J = 10.2, 5.1 Hz, 1H), 7.07 (t, J = 7.0 Hz, 1H), 6.40 (d, J = 15.5 Hz, 1H), 6.09 (dd, J = 4.8, 0.5 Hz, 1H), 3.86 (s, 3H), 3.85-3.79 (m, 2H), 3.62-3.56 (m, 2H), 3.42 (dd, J = 5.0, 1.8 Hz, 2H), 2.69 (s, 3H), 1.57 (t, J = 5.3 Hz, 4H), 1.17 (s, 3H) | 666.6 |
| 27-1, 27-2 | 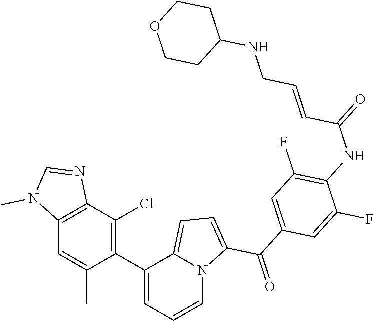 [SMILES: Cc1cc2c(ncn2C)c(Cl)c1-c1cccn2c(C(=O)c3cc(F)c(NC(=O)/C=C/CNC4CCOCC4)c(F)c3)ccc12] | (E)-N-(4-(8-(4-chloro-1,6- dimethyl-1H-benzo[d]imidazol- 5-yl)indolizine-3-carbonyl)-2,6- difluorophenyl)-4-((tetrahydro- 2H-pyran-4-yl)amino)but-2- enamide (1^{st} eluting isomer and 2^{nd} eluting isomer) | 1H-NMR (400 MHz, CHLOROFORM-d) δ 10.00-9.98 (m, 1H), 7.94 (s, 1H), 7.46-7.42 (m, 2H), 7.33-7.32 (m, 2H), 7.29 (d, J = 0.8 Hz, 1H), 7.18-7.09 (m, 3H), 6.29 (dt, J = 15.3, 1.8 Hz, 1H), 6.06 (dd, J = 4.9, 0.6 Hz, 1H), 3.99 (dt, J = 11.4, 3.2 Hz, 2H), 3.90 (s, 3H), 3.53 (dd, J = 5.3, 1.8 Hz, 2H), 3.41 (td, J = 11.7, 2.1 Hz, 2H), 2.78- 2.72 (m, 1H), 2.20 (d, J = 0.5 Hz, 3H), 1.86 (dt, J = 10.6, 1.9 Hz, 2H), 1.48-1.38 (m, 2H) | 618.5 |
| 28-1, 28-2 | 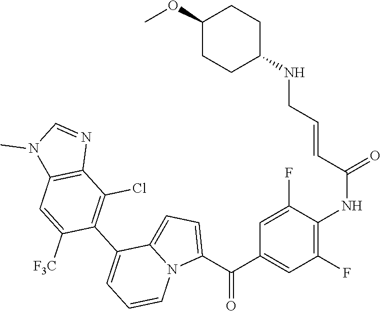 [SMILES: CO[C@H]1CC[C@H](NC/C=C/C(=O)Nc2c(F)cc(C(=O)c3ccc4c(-c5c(C(F)(F)F)cc6c(ncn6C)c5Cl)cccn34)cc2F)CC1] | (E)-N-(4-(8-(4-chloro-1- methyl-6-(trifluoromethyl)-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 10.01 (dt, J = 7.0, 0.9 Hz, 1H), 8.15 (s, 1H), 7.87 (s, 1H), 7.45 (dd, J = 10.5, 2.8 Hz, 2H), 7.33 (d, J = 4.8 Hz, 1H), 7.19-7.08 (m, 4H), 6.25 (dt, J = 15.3, 1.6 Hz, 1H), 6.01 (dd, J = 4.9, 0.6 Hz, 1H), 4.02 (s, 3H), 3.50 (dd, J = 5.3, 1.8 Hz, 2H), 3.35 (s, 3H), 3.17-3.14 (m, 1H), 2.59-2.52 (m, 1H), 2.10- 1.97 (m, 4H), 1.28-1.23 (m, 4H) | 700.4 |
| 29 | 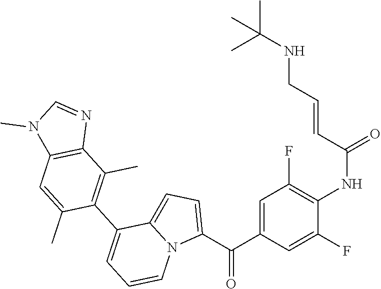 [SMILES: Cc1cc2c(ncn2C)c(C)c1-c1cccn2c(C(=O)c3cc(F)c(NC(=O)/C=C/CNC(C)(C)C)c(F)c3)ccc12] | (E)-4-(tert-butylamino)-N-(2,6- difluoro-4-(8-(1,4,6-trimethyl- 1H-benzo[d]imidazol-5- yl)indolizine-3- carbonyl)phenyl)but-2-enamide | 1H-NMR (400 MHz, DMSO-d6) δ 9.98 (s, 1H), 9.88 (d, J = 7.0 Hz, 1H), 8.14 (s, 1H), 7.56 (s, 1H), 7.54 (s, 1H), 7.40-7.39 (m, 2H), 7.30 (t, J = 6.9 Hz, 1H), 7.22 (dd, J = 7.0, 1.0 Hz, 1H), 6.89 (td, J = 10.3, 5.3 Hz, 1H), 6.37 (d, J = 15.3 Hz, 1H), 5.97 (d, J = 4.8 Hz, 1H), 3.84 (s, 3H), 2.19 (s, 3H), 2.07 (s, 3H), 1.07 (brs, 9H) | 570.4 |
| 30-1, 30-2 | 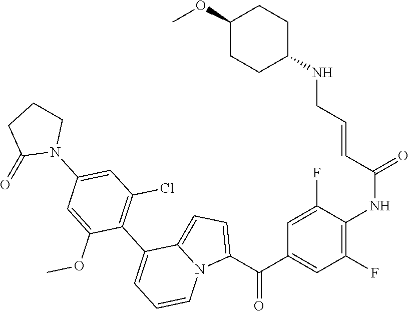 [SMILES: COc1cc(N2CCCC2=O)cc(Cl)c1-c1cccn2c(C(=O)c3cc(F)c(NC(=O)/C=C/CN[C@H]4CC[C@H](OC)CC4)c(F)c3)ccc12] | (E)-N-(4-(8-(2-chloro-6- methoxy-4-(2-oxopyrrolidin-1- yl)phenyl)indolizine-3- carbonyl)-2,6-difluorophenyl)- 4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.97 (d, J = 7.0 Hz, 1H), 7.77 (s, 1H), 7.40 (dd, J = 7.9, 0.9 Hz, 2H), 7.33 (d, J = 4.8 Hz, 1H), 7.16-7.07 (m, 4H), 6.28 (dd, J = 15.3, 1.0 Hz, 1H), 6.16 (d, J = 5.0 Hz, 1H), 3.92 (t, J = 7.0 Hz, 2H), 3.72 (s, 3H), 3.50 (d, J = 5.5 Hz, 2H), 3.35 (d, J = 1.0 Hz, 3H), 3.17-3.12 (m, 1H), 2.68 (t, J = 8.1 Hz, 2H), 2.56-2.51 (m, 1H), 2.26-2.19 (m, 2H), 2.09-1.97 (m, 4H), 1.28-1.13 (m, 4H) | 691.6 |
| 31 | 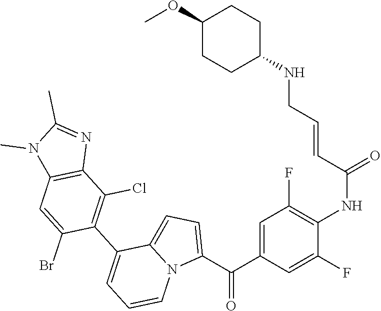 [SMILES: CO[C@H]1CC[C@H](NC/C=C/C(=O)Nc2c(F)cc(C(=O)c3ccc4c(-c5c(Br)cc6c(nc(C)n6C)c5Cl)cccn34)cc2F)CC1] | (E)-N-(4-(8-(6-bromo-4-chloro- 1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 10.00 (d, J = 6.8 Hz, 1H), 7.63 (s, 1H), 7.49-7.46 (m, 2H), 7.36-7.34 (m, 1H), 7.19-7.09 (m, 3H), 6.87 (s, 1H), 6.23 (d, J = 15.5 Hz, 1H), 6.09 (d, J = 4.8 Hz, 1H), 3.79 (s, 3H), 3.52-3.50 (m, 2H), 3.35 (s, 3H), 3.14 (d, J = 4.0 Hz, 1H), 2.69 (s, 3H), 2.58- 2.51 (m, 1H), 2.10-1.96 (m, 4H), 1.30-1.13 (m, 4H) | 726.3 |
| 32-1, 32-2 | 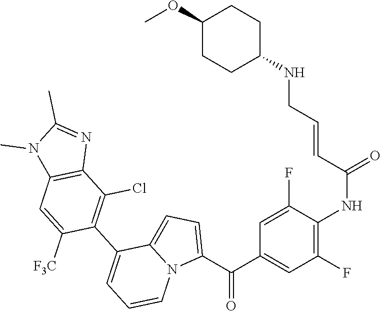 [SMILES: CO[C@H]1CC[C@H](NC/C=C/C(=O)Nc2c(F)cc(C(=O)c3ccc4c(-c5c(C(F)(F)F)cc6c(nc(C)n6C)c5Cl)cccn34)cc2F)CC1] | (E)-N-(4-(8-(4-chloro-1,2- dimethyl-6-(trifluoromethyl)- 1H-benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 10.00 (dt, J = 7.1, 0.9 Hz, 1H), 7.74 (s, 1H), 7.45 (dd, J = 10.5, 2.8 Hz, 2H), 7.32 (d, J = 4.8 Hz, 1H), 7.18- 7.16 (m, 1H), 7.14-7.08 (m, 2H), 6.26 (dd, J = 13.6, 1.6 Hz, 1H), 6.00 (dd, J = 4.8, 0.8 Hz, 1H), 3.88 (s, 3H), 3.51 (dd, J = 5.3, 1.8 Hz, 2H), 3.35 (s, 3H), 3.17-3.11 (m, 1H), 2.74 (s, 3H), 2.60- 2.52 (m, 1H), 2.10-1.97 (m, 4H), 1.28-1.15 (m, 4H) | 714.6 |
| 33 | 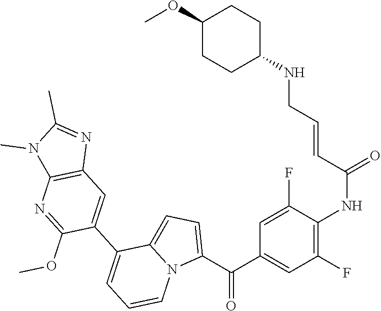 [SMILES: COc1nc2c(cc1-c1cccn3c(C(=O)c4cc(F)c(NC(=O)/C=C/CN[C@H]5CC[C@H](OC)CC5)c(F)c4)ccc13)nc(C)n2C] | (E)-N-(2,6-difluoro-4-(8-(5- methoxy-2,3-dimethyl-3H- imidazo[4,5-b]pyridin-6- yl)indolizine-3- carbonyl)phenyl)-4-(((1r,4r)-4- methoxycyclohexyl)amino)but- 2-enamide | 1H-NMR (400 MHz, CHLOROFORM-d) δ 9.98 (d, J = 7.0 Hz, 1H), 7.93 (t, J = 3.9 Hz, 1H), 7.52- 7.46 (m, 2H), 7.40-7.32 (m, 1H), 7.13-7.05 (m, 2H), 6.35 (d, J = 5.0 Hz, 1H), 6.27 (d, J = 15.3 Hz, 1H), 3.99 (s, 3H), 3.81 (s, 3H), 3.59-3.56 (m, 2H), 3.37 (s, 3H), 3.18-3.12 (m, 1H), 2.67 (s, 3H), 2.56-2.48 (m, 1H), 2.23-1.97 (m, 4H), 1.30- 1.18 (m, 4H) | 643.6 |
| 34-1, 34-2 | 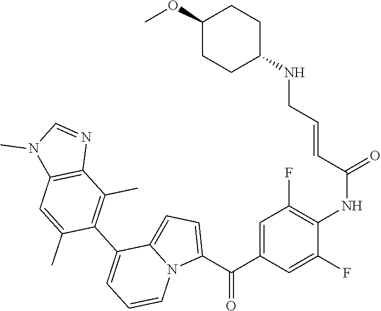 [SMILES: CO[C@H]1CC[C@H](NC/C=C/C(=O)Nc2c(F)cc(C(=O)c3ccc4c(-c5c(C)cc6c(ncn6C)c5C)cccn34)cc2F)CC1] | (E)-N-(2,6-difluoro-4-(8-(1,4,6- trimethyl-1H- benzo[d]imidazol-5- yl)indolizine-3- carbonyl)phenyl)-4-(((1r,4r)-4- methoxycyclohexyl)amino)but- 2-enamide (1^{st} eluting isomer and 2^{nd} eluting isomer) | 1H-NMR (400 MHz, DMSO-d6) δ 9.98 (s, 1H), 9.88 (d, J = 6.8 Hz, 1H), 8.14 (s, 1H), 7.56-7.53 (m, 2H), 7.40-7.39 (m, 2H), 7.30 (t, J = 7.0 Hz, 1H), 7.22 (dd, J = 6.9, 1.1 Hz, 1H), 6.86 (td, J = 10.3, 5.2 Hz, 1H), 6.33 (d, J = 15.5 Hz, 1H), 5.97 (d, J = 4.8 Hz, 1H), 3.84 (s, 3H), 3.43 (qd, J = 7.0, 5.0 Hz, 1H), 3.36 (d, J = 3.5 Hz, 2H), 3.21 (s, 3H), 3.09-3.04 (m, 1H), 2.19 (s, 3H), 2.07 (s, 3H), 1.96-1.86 (m, 4H), 1.13-1.02 (m, 4H) | 626.4 |
| 35-1, 35-2 | 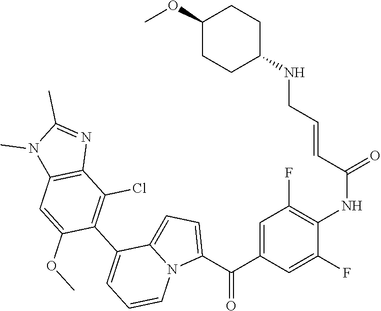 [SMILES: COc1cc2c(nc(C)n2C)c(Cl)c1-c1cccn2c(C(=O)c3cc(F)c(NC(=O)/C=C/CN[C@H]4CC[C@H](OC)CC4)c(F)c3)ccc12] | (E)-N-(4-(8-(4-chloro-6- methoxy-1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.94 (d, J = 6.5 Hz, 1H), 7.41 (d, J = 8.0 Hz, 2H), 7.27 (d, J = 5.3 Hz, 1H), 7.16 (d, J = 7.0 Hz, 1 H), 7.09-7.05 (m, 2H), 6.76 (s, 1H), 6.37-6.17 (m, 1H), 6.06 (d, J = 4.8 Hz, 1H), 3.74 (s, 3H), 3.71 (s, 3H), 3.51 (dd, J = 5.3, 1.8 Hz, 2H), 3.31 (s, 3H), 3.17-3.11 (m, 1H), 2.62 (s, 3H), 2.58-2.52 (m, 1H), 2.05-2.00 (m, 4H), 1.28-1.16 (m, 4H) | 676.6 |
| 36-1, 36-2 | 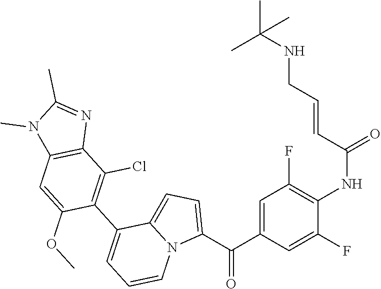 [SMILES: COc1cc2c(nc(C)n2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-6-methoxy-1,2- dimethyl-1H-benzo[d]imidazol- 5-yl)indolizine-3-carbonyl)-2,6- difluorophenyl)but-2-enamide (1^{st} eluting isomer and 2^{nd} eluting isomer) | 1H-NMR (400 MHz, CHLOROFORM-d) δ 9.97 (d, J = 7.0 Hz, 1H), 7.44 (d, J = 8.0 Hz, 2H), 7.31 (d, J = 4.8 Hz, 1H), 7.21-7.09 (m, 3H), 6.80 (s, 1H), 6.66-6.51 (m, 1H), 6.10 (d, J = 4.8 Hz, 1H), 3.78 (s, 3H), 3.75 (s, 3H), 3.60-3.56 (m, 2H), 2.66 (s, 3H), 1.15 (s, 9H) | 620.6 |
| 37 | 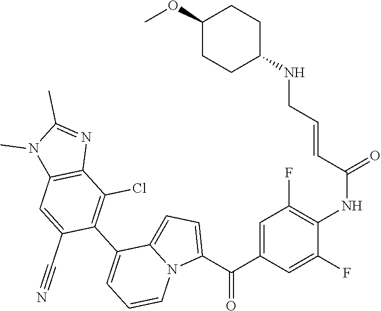 [SMILES: CO[C@H]1CC[C@H](NC/C=C/C(=O)Nc2c(F)cc(C(=O)c3ccc4c(-c5c(C#N)cc6c(nc(C)n6C)c5Cl)cccn34)cc2F)CC1] | (E)-N-(4-(8-(4-chloro-6-cyano- 1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 10.03 (d, J = 7.0 Hz, 1H), 7.73 (s, 1H), 7.49-7.43 (m, 1H), 7.39-7.36 (m, 1H), 7.29 (dd, J = 7.0, 1.0 Hz, 1H), 7.16-7.09 (m, 2H), 6.90 (s, 1H), 6.23 (d, J = 15.5 Hz, 1H), 6.11 (d, J = 4.8 Hz, 1H), 3.87 (s, 3H), 3.51 (dd, J = 5.3, 1.8 Hz, 2H), 3.34 (s, 3H), 3.19-3.07 (m, 1H), 2.76 (s, 3H), 2.62-2.50 (m, 1H), 2.10-1.97 (m, 4H), 1.30-1.09 (m, 4H) | 671.6 |
| 38 | 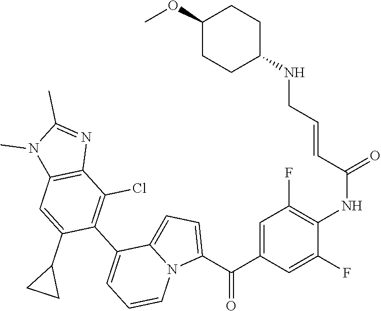 [SMILES: CO[C@H]1CC[C@H](NC/C=C/C(=O)Nc2c(F)cc(C(=O)c3ccc4c(-c5c(C6CC6)cc6c(nc(C)n6C)c5Cl)cccn34)cc2F)CC1] | (E)-N-(4-(8-(4-chloro-6- cyclopropyl-1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.99 (d, J = 7.0 Hz, 1H), 7.46 (d, J = 8.0 Hz, 2H), 7.33-7.30 (m, 1H), 7.23 (d, J = 6.8 Hz, 1H), 7.15- 7.08 (m, 2H), 6.91 (s, 1H), 6.25 (d, J = 15.3 Hz, 1H), 6.10 (d, J = 4.8 Hz, 1H), 3.77 (s, 3H), 3.52- 3.49 (m, 2H), 3.34 (s, 3H), 3.18-3.07 (m, 1H), 2.67 (s, 3H), 2.59-2.53 (m, 1H), 2.10-1.98 (m, 4H), 1.30-1.12 (m, 4H), 0.97-0.54 (m, 4H) | 686.5 |
| 39-1, 39-2 | 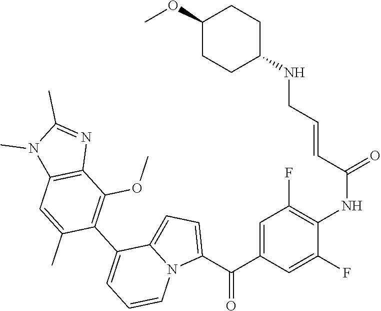 [SMILES: COc1c(-c2cccn3c(C(=O)c4cc(F)c(NC(=O)/C=C/CN[C@H]5CC[C@H](OC)CC5)c(F)c4)ccc23)c(C)cc2c1nc(C)n2C] | (E)-N-(2,6-difluoro-4-(8-(4- methoxy-1,2,6-trimethyl-1H- benzo[d]imidazol-5- yl)indolizine-3- carbonyl)phenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.96- 9.94 (m, 1H), 7.46-7.41 (m, 2H), 7.30 (d, J = 4.8 Hz, 1H), 7.15-7.08 (m, 4H), 6.93 (d, J = 0.5 Hz, 1H), 6.24 (d, J = 15.5 Hz, 1H), 6.11 (dd, J = 4.8, 0.8 Hz, 1H), 4.08 (s, 3H), 3.73 (s, 3H), 3.50 (dd, J = 5.3, 1.8 Hz, 2H), 3.35 (s, 3H), 3.18-3.11 (m, 1H), 2.62 (s, 3H), 2.59-2.50 (m, 1H), 2.12 (d, J = 0.5 Hz, 3H), 2.09-2.04 (m, 2H), 2.01-1.97 (m, 2H), 1.35-1.08 (m, 4H) | 656.7 |
| 40-1, 40-2 | 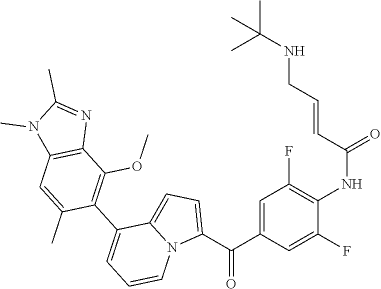 [SMILES: COc1c(-c2cccn3c(C(=O)c4cc(F)c(NC(=O)/C=C/CNC(C)(C)C)c(F)c4)ccc23)c(C)cc2c1nc(C)n2C] | (E)-4-(tert-butylamino)-N-(2,6- difluoro-4-(8-(4-methoxy- 1,2,6-trimethyl-1H- benzo[d]imidazol-5- yl)indolizine-3- carbonyl)phenyl)but-2-enamide (1^{st} eluting isomer and 2^{nd} eluting isomer) | 1H-NMR (400 MHz, CHLOROFORM-d) δ 9.95 (d, J = 6.5 Hz, 1H), 7.45-7.42 (m, 2H), 7.30 (d, J = 4.8 Hz, 1H), 7.18-7.07 (m, 4H), 6.93 (d, J = 0.8 Hz, 1H), 6.27 (dt, J = 15.3, 1.8 Hz, 1H), 6.11 (dd, J = 4.8, 0.5 Hz, 1H), 4.08 (s, 3H), 3.73 (s, 3H), 3.45 (dd, J = 5.1, 1.9 Hz, 2H), 2.62 (s, 3H), 2.12 (s, 3H), 1.15 (s, 9H) | 600.6 |
| 41-1, 41-2 | 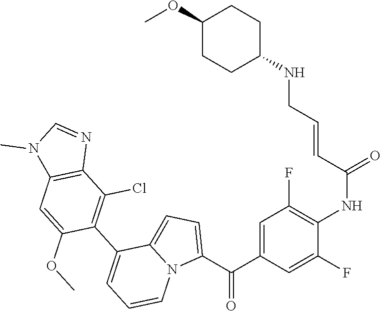 [SMILES: COc1cc2c(ncn2C)c(Cl)c1-c1cccn2c(C(=O)c3cc(F)c(NC(=O)/C=C/CN[C@H]4CC[C@H](OC)CC4)c(F)c3)ccc12] | (E)-N-(4-(8-(4-chloro-6- methoxy-1-methyl-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.99 (d, J = 7.0 Hz, 1H), 7.89 (s, 1H), 7.45 (d, J = 7.8 Hz, 2H), 7.32 (d, J = 4.8 Hz, 1H), 7.21 (d, J = 6.3 Hz, 1H), 7.14-7.08 (m, 2H), 6.98 (d, J = 12.3 Hz, 1H), 6.88 (s, 1H), 6.23 (d, J = 15.5 Hz, 1H), 6.10 (d, J = 4.8 Hz, 1H), 3.90 (s, 3H), 3.77 (s, 3H), 3.51-3.50 (m, 2H), 3.35 (s, 3H), 3.18-3.11 (m, 1H), 2.62-2.50 (m, 1H), 2.17-1.97 (m, 4H), 1.35- 1.10 (m, 4H) | 662.4 |
| 42 | 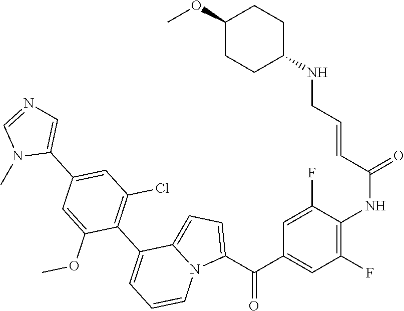 [SMILES: COc1cc(-c2cncn2C)cc(Cl)c1-c1cccn2c(C(=O)c3cc(F)c(NC(=O)/C=C/CN[C@H]4CC[C@H](OC)CC4)c(F)c3)ccc12] | (E)-N-(4-(8-(2-chloro-6- methoxy-4-(1-methyl-1H- imidazol-5- yl)phenyl)indolizine-3- carbonyl)-2,6-difluorophenyl)- 4-(((1r,4r)-4- methoxycyclohexyl)amino)but- 2-enamide | 1H-NMR (400 MHz, CHLOROFORM-d) δ 9.99 (dt, J = 7.0, 0.9 Hz, 1H), 7.59 (d, J = 6.0 Hz, 1H), 7.44 (d, J = 8.3 Hz, 2H), 7.36 (d, J = 4.8 Hz, 1H), 7.23 (d, J = 1.0 Hz, 1H), 7.21-7.17 (m, 2H), 7.14- 7.08 (m, 2H), 6.95 (d, J = 1.5 Hz, 1H), 6.27 (d, J = 15.3 Hz, 1H), 6.21 (dd, J = 4.8 Hz, 0.8 Hz, 1H), 3.79 (s, 3H), 3.75 (s, 3H), 3.53-3.48 (m, 2H), 3.37 (s, 3H), 3.19-3.11 (m, 1H), 2.59-2.50 (m, 1H), 2.11-1.97 (m, 4H), 1.21 (m, 4H) | 688.4 |
| 43 | 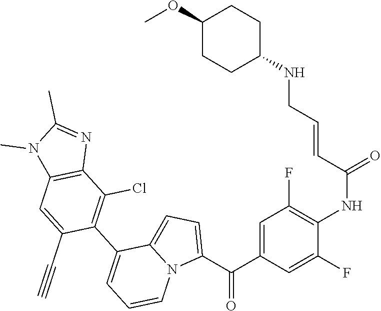 [SMILES: C#Cc1cc2c(nc(C)n2C)c(Cl)c1-c1cccn2c(C(=O)c3cc(F)c(NC(=O)/C=C/CN[C@H]4CC[C@H](OC)CC4)c(F)c3)ccc12] | (E)-N-(4-(8-(4-chloro-6- ethynyl-1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 10.00 (d, J = 7.0 Hz, 1H), 7.56 (s, 1H), 7.52-7.46 (m, 2H), 7.36-7.33 (m, 1H), 7.25-7.24 (m, 1H), 7.15- 7.08 (m, 2H), 6.26 (d, J = 15.0 Hz, 1H), 6.12 (d, J = 4.8 Hz, 1H), 3.81 (s, 3H), 3.53-3.51 (m, 2H), 3.35 (s, 3H), 3.18-3.07 (m, 1H), 2.83 (s, 1H), 2.70 (s, 3H), 2.62-2.53 (m, 1H), 2.10-1.99 (m, 4H), 1.30-1.17 (m, 4H) | 670.5 |
| 44 | 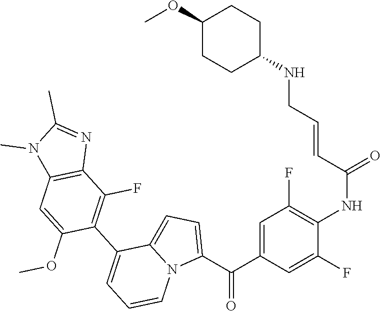 [SMILES: COc1cc2c(nc(C)n2C)c(F)c1-c1cccn2c(C(=O)c3cc(F)c(NC(=O)/C=C/CN[C@H]4CC[C@H](OC)CC4)c(F)c3)ccc12] | (E)-N-(2,6-difluoro-4-(8-(4- fluoro-6-methoxy-1,2- dimethyl-1H-benzo[d]imidazol- 5-yl)indolizine-3- carbonyl)phenyl)-4-(((1r,4r)-4- methoxycyclohexyl)amino)but- 2-enamide | 1H-NMR (400 MHz, DMSO-d6) δ 9.99 (s, 1H), 9.86 (d, J = 6.8 Hz, 1H), 7.56-7.52 (m, 2H), 7.40 (d, J = 4.8 Hz, 1H), 7.32 (dd, J = 7.0, 1.0 Hz, 1H), 7.25 (t, J = 7.0 Hz, 1H), 7.16 (s, 1H), 6.86 (dt, J = 10.3, 5.1 Hz, 1H), 6.33 (d, J = 15.5 Hz, 1H), 6.14 (d, J = 4.8 Hz, 1H), 3.78 (s, 3H), 3.75 (s, 3H), 3.38 (s, 2H), 3.21 (s, 3H), 3.10-3.06 (m, 1H), 2.53 (s, 3H), 2.34-2.44 (1H), 1.97-1.86 (m, 4H), 1.16- 1.02 (m, 4H) | 660.5 |
| 45 | 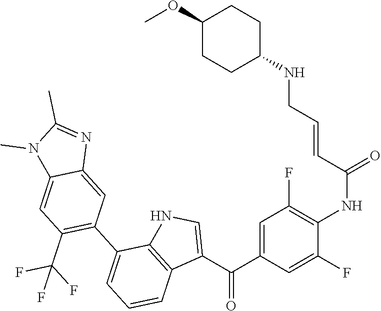 [SMILES: CO[C@H]1CC[C@H](NC/C=C/C(=O)Nc2c(F)cc(C(=O)c3c[nH]c4c(-c5cc6nc(C)n(C)c6cc5C(F)(F)F)cccc34)cc2F)CC1] | (E)-N-(4-(7-(1,2-dimethyl-6- (trifluoromethyl)-1H- benzo[d]imidazol-5-yl)-1H- indole-3-carbonyl)-2,6- difluorophenyl)-4-(((1r,4r)-4- methoxycyclohexyl)amino)but- 2-enamide | 1H-NMR (400 MHz, CHLOROFORM-d) δ 10.00 (d, J = 7.0 Hz, 1H), 7.56 (s, 1H), 7.52-7.46 (m, 2H), 7.36-7.33 (m, 1H), 7.25-7.24 (m, 1H), 7.15- 7.08 (m, 2H), 6.26 (d, J = 15.0 Hz, 1H), 6.12 (d, J = 4.8 Hz, 1H), 3.81 (s, 3H), 3.53-3.51 (m, 2H), 3.35 (s, 3H), 3.18-3.07 (m, 1H), 2.83 (s, 1H), 2.70 (s, 3H), 2.62-2.53 (m, 1H), 2.10-1.99 (m, 4H), 1.30-1.17 (m, 4H) | 680.6 |
| 46 | 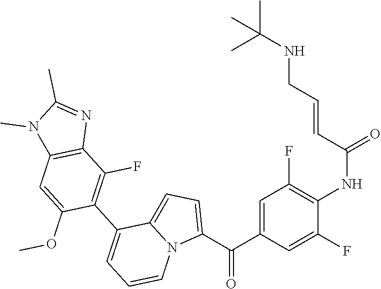 [SMILES: COc1cc2c(nc(C)n2C)c(F)c1-c1cccn2c(C(=O)c3cc(F)c(NC(=O)/C=C/CNC(C)(C)C)c(F)c3)ccc12] | (E)-4-(tert-butylamino)-N-(2,6- difluoro-4-(8-(4-fluoro-6- methoxy-1,2-dimethyl-1H- benzo[d]imidazol-5- yl)indolizine-3- carbonyl)phenyl)but-2-enamide | 1H-NMR (400 MHz, DMSO-d6) δ 10.09 (s, 1H), 9.86 (d, J = 6.8 Hz, 1H), 7.56-7.54 (m, 2H), 7.39 (d, J = 5.0 Hz, 1H), 7.32 (dd, J = 7.0, 1.0 Hz, 1H), 7.26 (t, J = 7.0 Hz, 1H), 7.17 (s, 1H), 6.89-6.85 (m, 1H), 6.45-6.40 (m, 1H), 6.15 (d, J = 4.8 Hz, 1H), 3.78 (s, 3H), 3.75 (s, 3H), 2.52 (s, 3H), 1.14 (brs, 9H) | 604.4 |
| 47 | 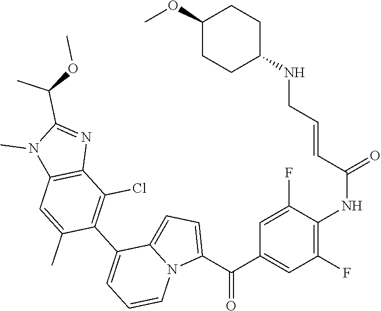 [SMILES: CO[C@H](C)c1nc2c(Cl)c(-c3cccn4c(C(=O)c5cc(F)c(NC(=O)/C=C/CN[C@H]6CC[C@H](OC)CC6)c(F)c5)ccc34)c(C)cc2n1C] | (E)-N-(4-(8-(4-chloro-2-((R)-1- methoxyethyl)-1,6-dimethyl- 1H-benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.99 (dd, J = 6.6, 0.6 Hz, 1H), 7.47-7.43 (m, 2H), 7.33 (d, J = 4.8 Hz, 1H), 7.25 (s, 1H), 7.17-7.08 (m, 3H), 6.25 (d, J = 15.5 Hz, 1H), 6.07 (d, J = 4.8 Hz, 1H), 4.96 (q, J = 6.8 Hz, 1H), 3.97 (s, 3H), 3.51 (dd, J = 5.1, 1.6 Hz, 2H), 3.36 (s, 3H), 3.35 (s, 3H), 3.17-3.12 (m, 1H), 2.56-2.51 (m, 1H), 2.20 (s, 3H), 2.09-1.97 (m, 4H), 1.70 (d, J = 7.0 Hz, 3H), 1.31-1.11 (m, 4H) | 704.6 |
| 48 | 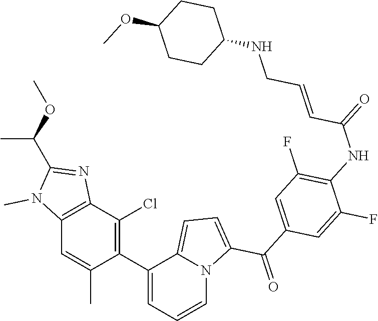 [SMILES: CO[C@H](C)c1nc2c(Cl)c(-c3cccn4c(C(=O)c5cc(F)c(NC(=O)/C=C/CN[C@H]6CC[C@H](OC)CC6)c(F)c5)ccc34)c(C)cc2n1C] | (E)-N-(4-(8-(4-chloro-2-((R)-1- methoxyethyl)-1,6-dimethyl- 1H-benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.99 (dd, J = 6.6, 0.6 Hz, 1H), 7.47-7.43 (m, 2H), 7.33 (d, J = 4.8 Hz, 1H), 7.25 (s, 1H), 7.17-7.08 (m, 3H), 6.25 (d, J = 15.5 Hz, 1H), 6.07 (d, J = 4.8 Hz, 1H), 4.96 (q, J = 6.8 Hz, 1H), 3.97 (s, 3H), 3.51 (dd, J = 5.1, 1.6 Hz, 2H), 3.36 (s, 3H), 3.35 (s, 3H), 3.17-3.12 (m, 1H), 2.56-2.51 (m, 1H), 2.20 (s, 3H), 2.09-1.97 (m, 4H), 1.70 (d, J = 7.0 Hz, 3H), 1.31-1.11 (m, 4H) | 704.6 |
| 49 | 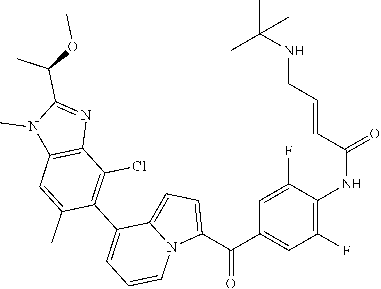 [SMILES: CO[C@H](C)c1nc2c(Cl)c(-c3cccn4c(C(=O)c5cc(F)c(NC(=O)/C=C/CNC(C)(C)C)c(F)c5)ccc34)c(C)cc2n1C] | (R,E)-4-(tert-butylamino)-N-(4- (8-(4-chloro-2-(1- methoxyethyl)-1,6-dimethyl- 1H-benzo[d]imidazol-5- yl)indolizine-3-carbonyl)-2,6- difluorophenyl)but-2-enamide (1^{st} eluting isomer) | 1H-NMR (400 MHz, CHLOROFORM-d) δ 9.99 (dd, J = 6.8, 0.8 Hz, 1H), 7.46 (d, J = 2.8 Hz, 1H), 7.43 (d, J = 2.8 Hz, 1H), 7.33 (d, J = 4.8 Hz, 1H), 7.25 (d, J = 0.8 Hz, 1H), 7.18-7.10 (m, 3H), 6.28 (dt, J = 15.3, 1.9 Hz, 1H), 6.07 (dd, J = 4.8, 0.8 Hz, 1H), 4.96 (q, J = 6.8 Hz, 1H), 3.97 (s, 3H), 3.45 (dd, J = 5.3, 1.8 Hz, 2H), 3.36 (s, 3H), 2.20 (s, 3H), 1.70 (d, J = 7.0 Hz, 3H), 1.16 (s, 9H) | 648.6 |
| 50 | 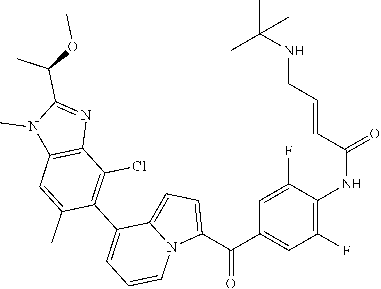 [SMILES: CO[C@H](C)c1nc2c(Cl)c(-c3cccn4c(C(=O)c5cc(F)c(NC(=O)/C=C/CNC(C)(C)C)c(F)c5)ccc34)c(C)cc2n1C] | (R,E)-4-(tert-butylamino)-N-(4- (8-(4-chloro-2-(1- methoxyethyl)-1,6-dimethyl- 1H-benzo[d]imidazol-5- yl)indolizine-3-carbonyl)-2,6- difluorophenyl)but-2-enamide (2^{nd} eluting isomer) | 1H-NMR (400 MHz, CHLOROFORM-d) δ 9.99 (dd, J = 6.8, 0.8 Hz, 1H), 7.46 (d, J = 2.8 Hz, 1H), 7.43 (d, J = 2.8 Hz, 1H), 7.33 (d, J = 4.8 Hz, 1H), 7.25 (d, J = 0.8 Hz, 1H), 7.18-7.10 (m, 3H), 6.28 (dt, J = 15.3, 1.9 Hz, 1H), 6.07 (dd, J = 4.8, 0.8 Hz, 1H), 4.96 (q, J = 6.8 Hz, 1H), 3.97 (s, 3H), 3.45 (dd, J = 5.3, 1.8 Hz, 2H), 3.36 (s, 3H), 2.20 (s, 3H), 1.70 (d, J = 7.0 Hz, 3H), 1.16 (s, 9H) | 648.6 |
| 51 | 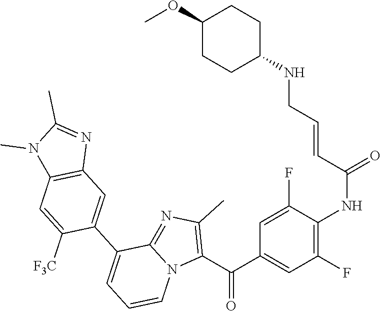 [SMILES: CO[C@H]1CC[C@H](NC/C=C/C(=O)Nc2c(F)cc(C(=O)c3c(C)nc4c(-c5cc6nc(C)n(C)c6cc5C(F)(F)F)cccn34)cc2F)CC1] | (E)-N-(4-(8-(1,2-dimethyl-6- (trifluoromethyl)-1H- benzo[d]imidazol-5-yl)-2- methylimidazo[1,2-a]pyridine- 3-carbonyl)-2,6- difluorophenyl)-4-(((1r,4r)-4- methoxycyclohexyl)amino)but- 2-enamide | 1H-NMR (400 MHz, CHLOROFORM-d) δ = 9.51 (d, J = 6.8 Hz, 1H), 7.73-7.69 (m, 2H), 7.46 (d, J = 7.1 Hz, 1H), 7.32 (d, J = 8.0 Hz, 2H), 7.12-7.02 (m, 2H), 6.21 (d, J = 15.4 Hz, 1H), 3.83 (s, 3H), 3.48 (brs, 2H), 3.35 (s, 3H), 3.14 (s, 3H), 2.67 (s, 3H), 2.17 (s, 3H), 2.12-2.00 (m, 4H), 1.27-1.17 (m, 4H) | 695.6 |
| 52 | 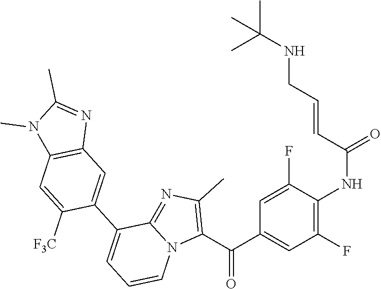 [SMILES: Cc1nc2c(-c3cc4nc(C)n(C)c4cc3C(F)(F)F)cccn2c1C(=O)c1cc(F)c(NC(=O)/C=C/CNC(C)(C)C)c(F)c1] | (E)-4-(tert-butylamino)-N-(4- (8-(1,2-dimethyl-6- (trifluoromethyl)-1H- benzo[d]imidazol-5-yl)-2- methylimidazo[1,2-a]pyridine- 3-carbonyl)-2,6- difluorophenyl)but-2-enamide | 1H-NMR (400 MHz, CHLOROFORM-d) δ 9.52 (d, J = 6.8 Hz, 1H), 7.74-7.70 (m, 2H), 7.45 (d, J = 7.0 Hz, 1H), 7.32 (d, J = 8.5 Hz, 2H), 7.14-7.07 (m, 2H), 6.25 (d, J = 15.3 Hz, 1H), 3.83 (s, 3H), 3.44 (brs, 2H), 2.67 (s, 3H), 2.18 (s, 3H), 1.14 (s, 9H) | 639.5 |
| 53 | 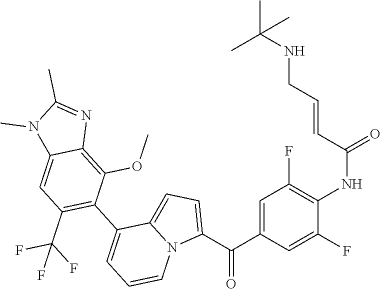 [SMILES: COc1c(-c2cccn3c(C(=O)c4cc(F)c(NC(=O)/C=C/CNC(C)(C)C)c(F)c4)ccc23)c(C(F)(F)F)cc2c1nc(C)n2C] | (E)-4-(tert-butylamino)-N-(2,6- difluoro-4-(8-(4-methoxy-1,2- dimethyl-6-(trifluoromethyl)- 1H-benzo[d]imidazol-5- yl)indolizine-3- carbonyl)phenyl)but-2-enamide | 1H-NMR (400 MHz, CHLOROFORM-d) δ 9.96 (d, J = 7.0 Hz, 1H), 7.52 (s, 1H), 7.46-7.44 (m, 3H), 7.31-7.29 (m, 1H), 7.17-7.06 (m, 2 H), 6.27 (d, J 15.3 Hz, 1H), 6.05 (d, J = 4.8 Hz, 1H), 4.13 (s, 3H), 3.82 (s, 3H), 3.46-3.45 (m, 2H), 2.68 (s, 3H), 1.16 (s, 9H) | 654.5 |
| 54 | 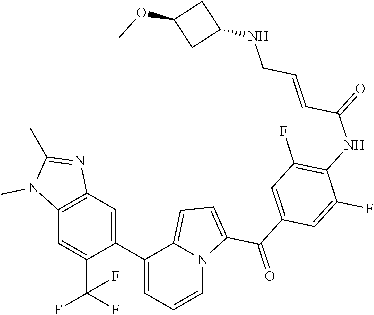 [SMILES: CO[C@H]1C[C@H](NC/C=C/C(=O)Nc2c(F)cc(C(=O)c3ccc4c(-c5cc6nc(C)n(C)c6cc5C(F)(F)F)cccn34)cc2F)C1] | (E)-N-(4-(8-(1,2-dimethyl-6- (trifluoromethyl)-1H- benzo[d]imidazol-5- yl)indolizine-3-carbonyl)-2,6- difluorophenyl)-4-(((1r,3r)-3- methoxycyclobutyl)amino)but- 2-enamide | 1H-NMR (400 MHz, CHLOROFORM-d) δ 9.97 (d, J = 7.0 Hz, 1H), 7.78 (s, 1H), 7.68 (s, 1H), 7.45 (t, J = 2.5 Hz, 1H), 7.42 (t, J = 2.5 Hz, 1H), 7.30 (t, J = 4.6 Hz, 1H), 7.19 (d, J = 7.0 Hz, 1H), 7.13-7.05 (m, 2H), 6.27 (d, J = 15.5 Hz, 1H), 6.10 (d, J = 4.8 Hz, 1H), 4.07-4.04 (m, 1H), 3.87 (s, 3H), 3.54-3.50 (m, 1H), 3.41 (dd, J = 5.3, 1.8 Hz, 2H), 3.24 (s, 3H), 2.70 (s, 3H), 2.26-2.20 (m, 2H), 2.10-2.03 (m, 2H) | 652.6 |
| 55 | 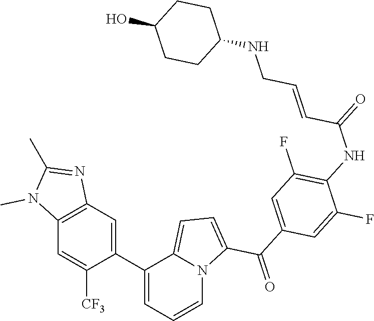 [SMILES: Cc1nc2cc(-c3cccn4c(C(=O)c5cc(F)c(NC(=O)/C=C/CN[C@H]6CC[C@H](O)CC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-(((1r,4r)-4- hydroxycyclohexyl)amino)but- 2-enamide | 1H-NMR (400 MHz, CHLOROFORM-d) δ 9.96 (d, J = 7.3 Hz, 1H), 7.77 (s, 1H), 7.68 (s, 1H), 7.42 (d, J = 8.0 Hz, 2H), 7.30 (d, J = 4.8 Hz, 1H), 7.19 (d, J = 7.0 Hz, 1H), 7.11-7.05 (m, 2H), 6.27 (d, J = 15.3 Hz, 1H), 6.09 (d, J = 4.8 Hz, 1H), 3.86 (s, 3H), 3.67-3.58 (m, 1H), 3.51 (d, J = 4.5 Hz, 2H), 2.69 (s, 3H), 2.61-2.51 (m, 1H), 2.02- 1.95 (m, 4H), 1.37-1.18 (m, 4H) | 666.6 |
| 56-1, 56-2 | 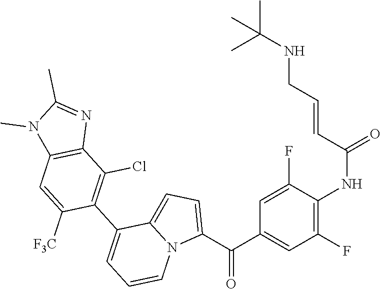 [SMILES: Cc1nc2c(Cl)c(-c3cccn4c(C(=O)c5cc(F)c(NC(=O)/C=C/CNC(C)(C)C)c(F)c5)ccc34)c(C(F)(F)F)cc2n1C] | (E)-4-(tert-butylamino)-N-(4- (8-(4-chloro-1,2-dimethyl-6- (trifluoromethyl)-1H- benzo[d]imidazol-5- yl)indolizine-3-carbonyl)-2,6- difluorophenyl)but-2-enamide (1^{st} eluting isomer and 2^{nd} eluting isomer) | 1H-NMR (400 MHz, CHLOROFORM-d) δ 10.00 (d, J =7.0 Hz, 1H), 7.74 (s, 1H), 7.44 (d, J = 7.8, 2H), 7.32 (d, J = 4.8 Hz, 1H), 7.19-7.09 (m, 3H), 6.30 (d, J = 15.5 Hz, 1H), 6.00 (d, J = 5.0 Hz, 1H), 3.88 (s, 3H), 3.45 (dd, J = 5.3, 1.5 Hz, 2H), 2.74 (s, 3H), 1.15 (s, 9H) | 658.5 |
| 57-1, 57-2 | 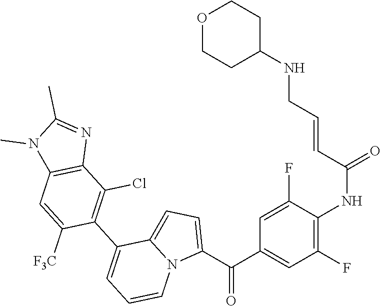 [SMILES: Cc1nc2c(Cl)c(-c3cccn4c(C(=O)c5cc(F)c(NC(=O)/C=C/CNC6CCOCC6)c(F)c5)ccc34)c(C(F)(F)F)cc2n1C] | (E)-N-(4-(8-(4-chloro-1,2- dimethyl-6-(trifluoromethyl)- 1H-benzo[d]imidazol-5- yl)indolizine-3-carbonyl)-2,6- difluorophenyl)-4-((tetrahydro- 2H-pyran-4-yl)amino)but-2- enamide (1^{st} eluting isomer and 2^{nd} eluting isomer) | 1H-NMR (400 MHz, CHLOROFORM-d) δ 10.00 (dt, J = 7.0, 1.0 Hz, 1H), 7.74 (s, 1H), 7.49-7.45 (m, 2H), 7.33 (d, J = 5.0 Hz, 1 H), 7.18-7.09 (m, 3H), 6.98 (brs, 1H), 6.25 (d, J = 15.5 Hz, 1H), 6.00 (dd, J = 4.8, 0.8 Hz, 1H), 4.02-3.98 (m, 2H), 3.88 (s, 3H), 3.53 (dd, J = 5.1, 1.9 Hz, 2H), 3.42 (td, J = 11.7, 2.2 Hz, 2H), 2.75 (s, 3H), 2.61 (d, J = 18.8 Hz, 1H), 1.89-1.83 (m, 2H) | 686.9 |
| 58 | 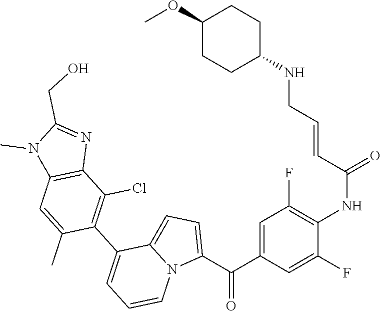 [SMILES: CO[C@H]1CC[C@H](NC/C=C/C(=O)Nc2c(F)cc(C(=O)c3ccc4c(-c5c(C)cc6c(nc(CO)n6C)c5Cl)cccn34)cc2F)CC1] | (E)-N-(4-(8-(4-chloro-2- (hydroxymethyl)-1,6-dimethyl- 1H-benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.98 (dd, J = 6.3, 1.0 Hz, 1H), 7.46-7.41 (m, 2H), 7.30 (t, J = 4.5 Hz, 1H), 7.25 (s, 1H), 7.15-7.07 (m, 3H), 6.30 (d, J = 15.5 Hz, 1H), 6.02 (d, J = 4.8 Hz, 1H), 4.99 (s, 2H), 3.90 (s, 3H), 3.53-3.52 (m, 2H), 3.35 (s, 3H), 3.17-3.12 (m, 1H), 2.62-2.52 (m, 1H), 2.19 (s, 3H), 2.09-1.98 (m, 4H), 1.35- 1.12 (m, 4H) | 676.4 |
| 59 | 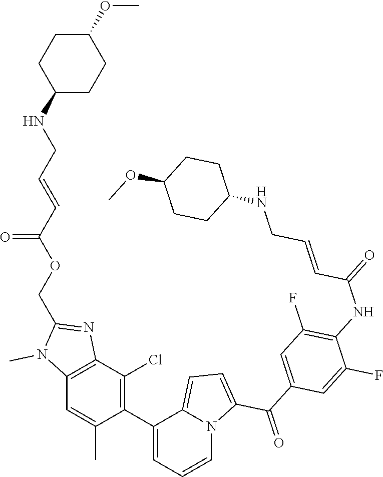 [SMILES: CO[C@H]1CC[C@H](NC/C=C/C(=O)Nc2c(F)cc(C(=O)c3ccc4c(-c5c(C)cc6c(nc(COC(=O)/C=C/CN[C@H]7CC[C@H](OC)CC7)n6C)c5Cl)cccn34)cc2F)CC1] | (4-chloro-5-(3-(3,5-difluoro-4- ((E)-4-(((1r,4r)-4- methoxycyclohexyl)amino)but- 2-enamido)benzoyl)indolizin-8- yl)-1,6-dimethyl-1H- benzo[d]imidazol-2-yl)methyl (E)-4-(((1r,4r)-4- methoxycyclohexyl)amino)but- 2-enoate | 1H-NMR (400 MHz, CHLOROFORM-d) δ 9.99 (dq, 6.6, 0.8 Hz, 1H), 7.48-7.43 (m, 2H), 7.35- 7.30 (m, 2H), 7.16-7.06 (m, 4H), 6.28-6.19 (m, 1H), 6.08-6.02 (m, 2H), 5.51-5.47 (m, 2H), 3.86 (d, J = 10.8 Hz, 3H), 3.55-3.45 (m, 4H), 3.35 (s, 3H), 3.34 (s, 3H), 3.18-3.07 (m, 2H), 2.62-2.45 (m, 2H), 2.09-1.88 (m, 8H), 1.35-1.06 (m, 8H) | 871.6 |
| 60-1, 60-2 | 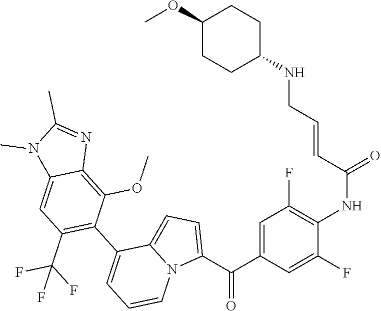 [SMILES: COc1c(-c2cccn3c(C(=O)c4cc(F)c(NC(=O)/C=C/CN[C@H]5CC[C@H](OC)CC5)c(F)c4)ccc23)c(C(F)(F)F)cc2c1nc(C)n2C] | (E)-N-(2,6-difluoro-4-(8-(4- methoxy-1,2-dimethyl-6- (trifluoromethyl)-1H- benzo[d]imidazol-5- yl)indolizine-3- carbonyl)phenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.97 (d, J = 7.0 Hz, 1H), 7.46-7.44 (m, 3H), 7.31-7.29 (m 1H), 7.16-7.06 (m, 3H), 6.94 (s, 1H), 6.22 (d, J = 15.3 Hz, 1H), 6.05 (d, J = 4.5 Hz, 1H), 4.14 (s, 3H), 3.82 (s, 3H), 3.50 (dd, J = 5.1, 1.6 Hz, 2H), 3.36 (s, 3H), 3.18-3.11 (m, 1H), 2.68 (s, 3H), 2.57-2.50 (m, 1H), 2.09-1.97 (m, 4H), 1.32- 1.09 (m, 4H) | 710.4 |
| 61-1, 61-2 | 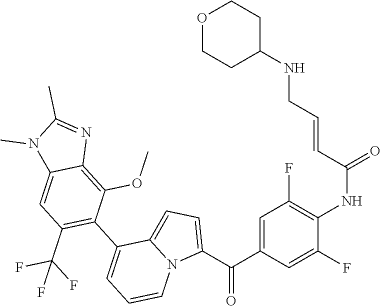 [SMILES: COc1c(-c2cccn3c(C(=O)c4cc(F)c(NC(=O)/C=C/CNC5CCOCC5)c(F)c4)ccc23)c(C(F)(F)F)cc2c1nc(C)n2C] | (E)-N-(2,6-difluoro-4-(8-(4- methoxy-1,2-dimethyl-6- (trifluoromethyl)-1H- benzo[d]imidazol-5- yl)indolizine-3- carbonyl)phenyl)-4- ((tetrahydro-2H-pyran-4- yl)amino)but-2-enamide (1^{st} eluting isomer and 2^{nd} eluting isomer) | 1H-NMR (400 MHz, CHLOROFORM-d) δ 9.97 (d, J = 7.0 Hz, 1H), 7.48-7.44 (m, 3H), 7.31-7.29 (m, 1H), 7.16-7.06 (m, 3H), 6.94 (s, 1H), 6.25 (dd, J = 13.6, 1.9 Hz, 1H), 6.05 (dd, J = 4.8, 0.8 Hz, 1H), 4.13 (s, 3H), 4.02-3.97 (m, 2H), 3.83 (s, 3H), 3.53 (dd, J = 5.3, 1.8 Hz, 2H), 3.42 (td, J = 11.7, 2.2 Hz, 2H), 2.68 (s, 3H), 1.86 (dd, J = 12.5, 2.0 Hz, 2H), 1.48-1.38 (m, 2H) | 682.4 |
| 62 | 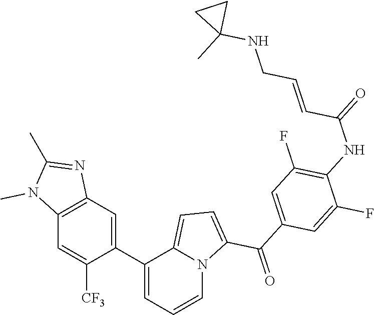 [SMILES: Cc1nc2cc(-c3cccn4c(C(=O)c5cc(F)c(NC(=O)/C=C/CNC6(C)CC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((1- methylcyclopropyl)amino)but- 2-enamide | 1H-NMR (400 MHz, CHLOROFORM-d) δ 9.97 (d, J = 7.3 Hz, 1H), 7.78 (s, 1H), 7.69 (s, 1H), 7.46-7.41 (m, 2H), 7.31 (d, J = 4.8 Hz, 1H), 7.20- 7.05 (m, 3H), 6.23 (d, J = 15.5 Hz, 1H), 6.10 (d, J = 4.8 Hz, 1H), 3.87 (s, 3H), 3.55 (dd, J = 5.0, 1.5 Hz, 2H), 2.70 (s, 3H), 1.29 (s, 3H), 0.62 (t, J = 5.0 Hz, 2H), 0.41 (dd, J = 6.0, 4.5 Hz, 2H) | 622.4 |
| 63 | 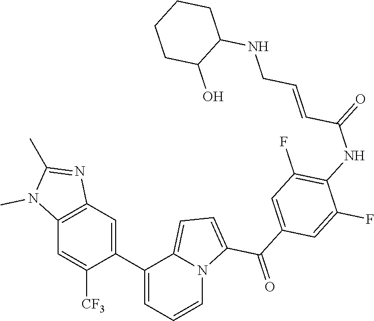 [SMILES: Cc1nc2cc(-c3cccn4c(C(=O)c5cc(F)c(NC(=O)/C=C/CNC6CCCCC6O)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((2- hydroxycyclohexyl)amino)but- 2-enamide | 1H-NMR (400 MHz, CHLOROFORM-d) δ 9.97 (dd, J = 7.2, 0.9 Hz, 1H), 7.78 (s, 1H), 7.69 (s, 1H), 7.46-7.42 (m, 2H), 7.32-7.30 (m, 2H), 7.19 (d, J = 7.0 Hz, 1H), 7.14-7.05 (m, 2H), 6.32-6.28 (m, 1H), 6.10 (dd, J = 4.8, 0.8 Hz, 1H), 3.87 (s, 3H), 3.75-3.60 (m, 1H), 3.50-3.44 (m, 1H), 3.29- 3.24 (m, 1H), 2.70 (s, 3H), 2.37-2.31 (m, 1H), 2.12-2.03 (m, 1H), 1.75-1.73 (m, 2H), 1.32-1.19 (m, 4H) | 666.5 |
| 64 | 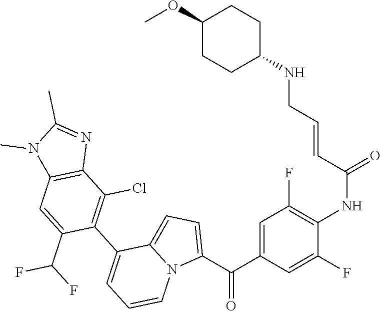 [SMILES: CO[C@H]1CC[C@H](NC/C=C/C(=O)Nc2c(F)cc(C(=O)c3ccc4c(-c5c(C(F)F)cc6c(nc(C)n6C)c5Cl)cccn34)cc2F)CC1] | (E)-N-(4-(8-(4-chloro-6- (difluoromethyl)-1,2-dimethyl- 1H-benzo[d]imidazol-5- yl)indolizine-3-carbonyl)-2,6- difluorophenyl)-4-(((1r,4)-4- methoxycyclohexyl)amino)but- 2-enamide | 1H-NMR (400 MHz, CHLOROFORM-d) δ 10.01 (d, J = 6.8 Hz, 1H), 7.71 (s, 1H), 7.49-7.45 (m, 2H), 7.35 (d, J = 4.8 Hz, 1H), 7.20 (d, J = 7.0 Hz, 1H), 7.15-7.08 (m, 2H), 6.25-6.19 (m, 1H), 6.05 (t, J = 4.5 Hz, 1H), 3.87 (s, 3H), 3.52-3.51 (m, 2H), 3.35 (s, 3H), 3.18-3.11 (m, 1H), 2.73 (s, 3H), 2.57-2.51 (m, 1H), 2.10-1.97 (m, 4H), 1.72- 1.13 (m, 4H) | 696.5 |
| 65 | 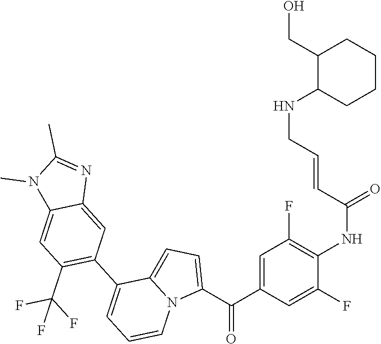 [SMILES: Cc1nc2cc(-c3cccn4c(C(=O)c5cc(F)c(NC(=O)/C=C/CNC6CCCCC6CO)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((2- (hydroxymethyl)cyclohexyl)ami- no)but-2-enamide | 1H-NMR (400 MHz, CHLOROFORM-d) δ 9.97 (d, J = 7.3 Hz, 1H), 7.78 (s, 1H), 7.69 (s, 1H), 7.45 (d, J = 8.0 Hz, 2H), 7.32 (d, J = 4.8 Hz, 1H), 7.19 (d, J = 7.0 Hz, 1H), 7.12-7.05 (m, 2H), 6.30 (d, J = 15.5 Hz, 1H), 6.10 (d, J = 4.8 Hz, 1H), 3.87 (s, 3H), 3.74 (dd, J = 16.9, 4.4 Hz, 1H), 3.67-3.59 (m, 2H), 3.48 (dd, J = 17.0, 3.8 Hz, 1H), 2.70 (s, 3H), 2.47 (dd, J = 14.1, 10.6 Hz, 1H), 2.23-2.20 (m, 1H), 1.82-1.64 (m, 4H), 1.26- 1.16 (m, 5H) | 680.5 |
| 66 | 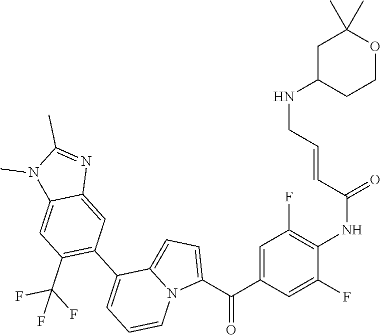 [SMILES: Cc1nc2cc(-c3cccn4c(C(=O)c5cc(F)c(NC(=O)/C=C/CNC6CCOC(C)(C)C6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((2,2- dimethyltetrahydro-2H-pyran- 4-yl)amino)but-2-enamide | 1H-NMR (400 MHz, CHLOROFORM-d) δ 9.97 (d, J = 7.0 Hz, 1H), 7.78 (s, 1H), 7.69 (s, 1H), 7.47-7.44 (m, 2H), 7.31 (d, J = 4.8 Hz, 1H), 7.19 (d, J = 7.0 Hz, 1H), 7.16-7.00 (m, 2H), 6.28 (d, J = 15.3 Hz, 1H), 6.10 (d, J = 4.8 Hz, 1H), 3.87 (s, 3H), 3.83-3.79 (m, 1H), 3.66 (td, J = 12.3, 2.3 Hz, 1H), 3.56-3.54 (m, 2H), 2.97-2.92 (m, 1H), 2.70 (s, 3H), 1.89-1.82 (m, 2H), 1.60-1.40 (m, 2H), 1.25 (s, 3H), 1.21 (s, 3H) | 680.5 |
| 67 | 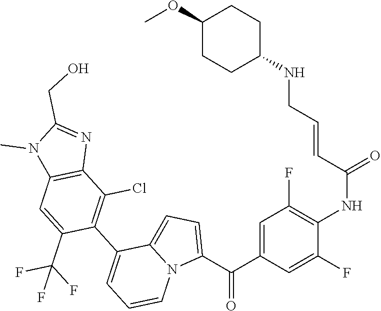 [SMILES: CO[C@H]1CC[C@H](NC/C=C/C(=O)Nc2c(F)cc(C(=O)c3ccc4c(-c5c(C(F)(F)F)cc6c(nc(CO)n6C)c5Cl)cccn34)cc2F)CC1] | (E)-N-(4-(8-(4-chloro-2- (hydroxymethyl)-1-methyl-6- (trifluoromethyl)-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 9.99 (dd, J = 6.9, 0.9 Hz, 1H), 7.82 (s, 1H), 7.44-7.41 (m, 2H), 7.29 (d, J = 4.8 Hz, 1H), 7.15-7.07 (m, 3H), 6.31 (d, J = 15.5 Hz, 1H), 5.94 (dd, J = 4.8, 0.8 Hz, 1H), 5.03 (dd, J = 18.5, 14.0 Hz, 2H), 4.02 (s, 3H), 3.50 (dd, J = 5.0, 1.8 Hz, 2H), 3.34 (s, 3H), 3.17-3.10 (m, 1H), 2.54 (tt, J = 10.4, 3.8 Hz, 1H), 2.08-1.88 (m, 4H), 1.28-1.09 (m, 4H) | 730.4 |
| 68 | 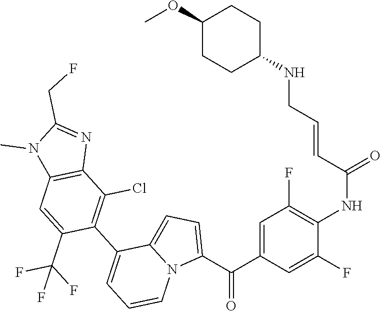 [SMILES: CO[C@H]1CC[C@H](NC/C=C/C(=O)Nc2c(F)cc(C(=O)c3ccc4c(-c5c(C(F)(F)F)cc6c(nc(CF)n6C)c5Cl)cccn34)cc2F)CC1] | (E)-N-(4-(8-(4-chloro-2- (fluoromethyl)-1-methyl-6- (trifluoromethyl)-1H- benzo[d]imidazol-5- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-d) δ 10.01 (d, J = 7.0 Hz, 1H), 7.87 (s, 1H), 7.46 (dt, J = 1 3.0, 2.6 Hz, 2H), 7.34 (d, J = 4.8 Hz, 1H), 7.18- 7.08 (m, 3H), 6.26 (d, J = 15.5 Hz, 1H), 5.99 (dd, J = 4.9, 0.6 Hz, 1H), 5.80 (d, J = 47.3 Hz, 2H), 4.05 (s, 3H), 3.52 (dd, J = 5.4, 1.6 Hz, 2H), 3.35 (s, 3H), 3.17-3.12 (m, 1H), 2.59-2.54 (m, 1H), 2.10-1.98 (m, 4H), 1.31-1.16 (m, 4H) | 732.4 |
| 69 | 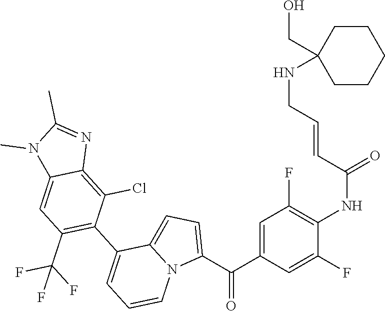 [SMILES: Cc1nc2c(Cl)c(-c3cccn4c(C(=O)c5cc(F)c(NC(=O)/C=C/CNC6(CO)CCCCC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((1- (hydroxymethyl)cyclohexyl)ami- no)but-2-enamide | 1H-NMR (400 MHz, CHLOROFORM-d) δ 9.97 (d, J = 7.0 Hz, 1H), 7.78 (s, 1H), 7.69 (s, 1H), 7.48-7.44 (m, 2H), 7.31 (d, J = 5.0 Hz, 1H), 7.20- 7.05 (m, 3H), 6.33 (d, J = 15.3 Hz, 1H), 6.10 (d, J = 4.8 Hz, 1H), 3.87 (s, 3H), 3.41-3.37 (m, 4H), 2.70 (s, 3H), 1.65-1.42 (m, 10H) | 680.5 |
| 70 | 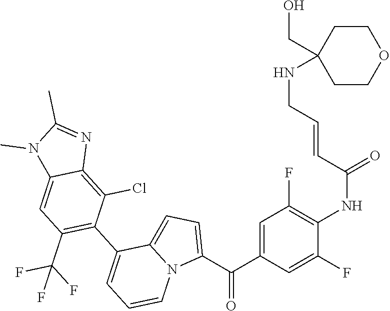 [SMILES: Cc1nc2c(Cl)c(-c3cccn4c(C(=O)c5cc(F)c(NC(=O)/C=C/CNC6(CO)CCOCC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((4- (hydroxymethyl)tetrahydro-2H- pyran-4-yl)amino)but-2- enamide | 1H-NMR (400 MHz, CHLOROFORM-d) δ 9.97 (d, J = 7.0 Hz, 1H), 7.78 (s, 1H), 7.69 (s, 1H), 7.51-7.43 (m, 2H), 7.32-7.31 (m, 1H), 7.22-7.05 (m, 3H), 6.33 (d, J = 15.5 Hz, 1H), 6.10 (d, J = 4.8 Hz, 1H), 3.87 (s, 3H), 3.79-3.74 (m, 2H), 3.71-3.67 (m, 2H), 3.52-3.48 (m, 2H), 3.42 (d, J = 3.5 Hz, 2H), 2.70 (s, 3H), 1.68-1.62 (m, 4H) | 682.6 |
| 71-1, 71-2 | 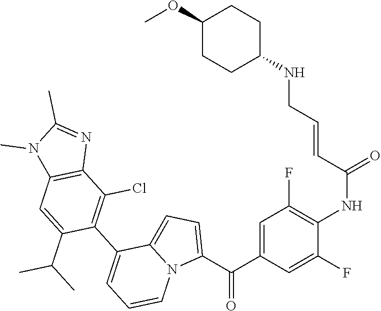 [SMILES: CO[C@H]1CC[C@H](NC/C=C/C(=O)Nc2c(F)cc(C(=O)c3ccc4c(-c5c(C(C)C)cc6c(nc(C)n6C)c5Cl)cccn34)cc2F)CC1] | (E)-N-(4-(8-(4-chloro-6- isopropyl-1,2-dimethyl-1H- benzo[d]imidazol-5- yl)indolizine-3-carbonyl)-2,6- difluorophenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.99- 9.86 (m, 1H), 7.48-7.44 (m, 2H), 7.32-7.29 (m, 1H), 7.25-7.23 (m, 1H), 7.22-7.03 (m, 4H), 6.25 (d, J = 15.5 Hz, 1H), 6.04 (dd, J = 4.8, 0.8 Hz, 1H), 3.81 (s, 3H), 3.54-3.58 (m, 2H), 3.35 (s, 3H), 3.18-3.11 (m, 1H), 2.75-2.67 (m, 4H), 2.58- 2.50 (m, 1H), 2.09-1.97 (m, 4H), 1.30-1.19 (m, 4H), 1.15 (d, J = 5.8 Hz, 3H), 1.14 (d, J = 5.8 Hz, 3H) | 688.4 |
| 72 | 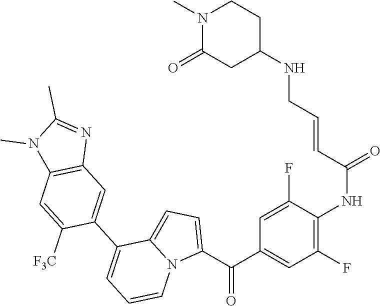 [SMILES: Cc1nc2cc(-c3cccn4c(C(=O)c5cc(F)c(NC(=O)/C=C/CNC6CCN(C)C(=O)C6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((1-methyl- 2-oxopiperidin-4-yl)amino)but- 2-enamide | 1H-NMR (400 MHz, CHLOROFORM-d) δ 9.96 (d, J = 7.0 Hz, 1H), 7.79 (s, 1H), 7.68 (s, 1H), 7.46-7.42 (m, 2H), 7.31 (d, J = 4.8 Hz, 1H), 7.19 (d, J = 7.0 Hz, 1H), 7.11-7.05 (m, 2H), 6.31 (d, J = 15.5 Hz, 1H), 6.10 (d, J = 4.8 Hz, 1H), 3.87 (s, 3H), 3.50 (d, J = 5.0 Hz, 2H), 3.41-3.22 (m, 2H), 3.12-3.06 (m, 1H), 2.94 (s, 3H), 2.70 (s, 3H), 2.25-2.17 (m, 1H), 2.10-2.00 (m, 1H), 1.75-1.67 (m, 2H) | 679.4 |
| 73 | 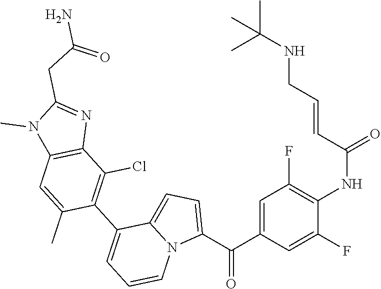 [SMILES: Cc1cc2c(nc(CC(N)=O)n2C)c(Cl)c1-c1cccn2c(C(=O)c3cc(F)c(NC(=O)/C=C/CNC(C)(C)C)c(F)c3)ccc12] | (E)-N-(4-(8-(2-(2-amino-2- oxoethyl)-4-chloro-1,6- dimethyl-1H-benzo[d]imidazol- 5-yl)indolizine-3-carbonyl)-2,6- difluorophenyl)-4-(tert- butylamino)but-2-enamide | 1H-NMR (400 MHz, CHLOROFORM-d) δ 9.98 (dd, J = 6.1, 2.1 Hz, 1H), 7.44 (d, J = 8.0 Hz, 2H), 7.34 (d, J = 4.8 Hz, 1H), 7.25 (d, J = 0.8 Hz, 1H), 7.16-7.11 (m, 2H), 7.02-6.94 (m, 1H), 6.48 (d, J = 15.5 Hz, 1H), 6.06 (d, J = 4.8 Hz, 1H), 3.94 (s, 2H), 3.91 (s, 3H), 3.60-3.54 (m, 2H), 2.20 (s, 3H), 1.31 (s, 9H) | 647.3 |
| 74 | 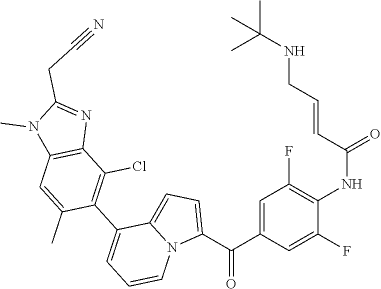 [SMILES: Cc1cc2c(nc(CC#N)n2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-2-(cyanomethyl)- 1,6-dimethyl-1H- benzo[d]imidazol-5- yl)indolizine-3-carbonyl)-2,6- difluorophenyl)but-2-enamide | 1H-NMR (400 MHz, CHLOROFORM-d) δ 9.99 (dd, J = 6.4, 0.8 Hz, 1H), 7.45 (d, J = 7.8 Hz, 2H), 7.33 (d, J = 4.8 Hz, 1H), 7.28-7.27 (m, 1H), 7.16- 7.09 (m, 3H), 6.36 (d, J = 15.3 Hz, 1H), 6.04 (dd, J = 4.8, 0.8 Hz, 1H), 4.19 (s, 2H), 3.95 (s, 3H), 3.50-3.48 (m, 2H), 2.21 (d, J = 0.8 Hz, 3H), 1.21 (s, 9H) | 629.4 |
| 75 | 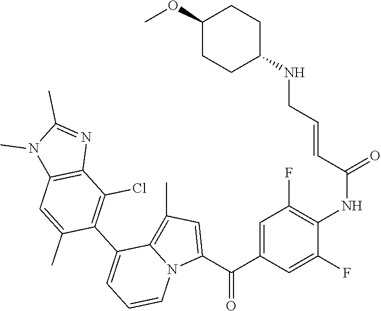 [SMILES: CO[C@H]1CC[C@H](NC/C=C/C(=O)Nc2c(F)cc(C(=O)c3cc(C)c4c(-c5c(C)cc6c(nc(C)n6C)c5Cl)cccn34)cc2F)CC1] | (E)-N-(4-(8-(4-chloro-1,2,6- trimethyl-1H- benzo[d]imidazol-5-yl)-1- isoindolizine-3-carbonyl)-2,6- difluorophenyl)-4-(((1r,4r)-4- methoxycyclohexyl)amino)but- 2-enamide | 1H NMR (400 MHz, CHLOROFORM-d) δ = 10.10 (dd, J = 5.9, 2.4 Hz, 1H), 7.53-7.48 (m, 2H), 7.32 (s, 1H), 7.18-7.08 (m, 3H), 6.87 (brs, 1H), 6.57 (s, 1H), 6.23 (brd, J = 15.4, 1.6 Hz, 1H), 3.64 (s, 3H), 3.51 (dd, J = 5.3, 1.5 Hz, 2H), 3.37 (s, 3H), 3.20-3.13 (m, 1H), 2.65 (s, 3H), 2.60-2.52 (m, 1H), 2.10-1.97 (m, 4H), 1.91 (s, 3H), 1.28-1.11 (m, 4H) | 786.1 |
| 76 | 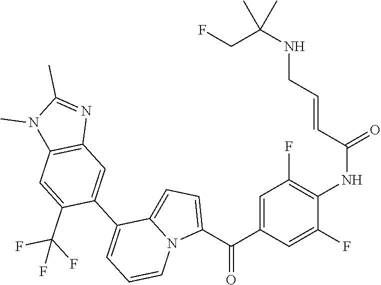 [SMILES: Cc1nc2cc(-c3cccn4c(C(=O)c5cc(F)c(NC(=O)/C=C/CNC(C)(C)CF)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((1-fluoro-2- methylpropan-2-yl)amino)but- 2-enamide | 1H-NMR (400 MHz, CHLOROFORM-d) δ 9.97 (d, J = 7.0 Hz, 1H), 7.78 (s, 1H), 7.69 (s, 1H), 7.45 (dt, J = 13.1, 2.6 Hz, 2H), 7.33-7.29 (m, 1H), 7.20-7.05 (m, 3H), 6.30 (d, J = 15.5 Hz, 1H), 6.10 (d, J = 4.8 Hz, 1H), 4.23 (d, J = 47.8 Hz, 2H), 3.87 (s, 3H), 3.47 (d, J = 3.5 Hz, 2H), 2.70 (s, 3H), 1.15 (s, 3H), 1.14 (s, 3H) | 642.5 |
| 77 | 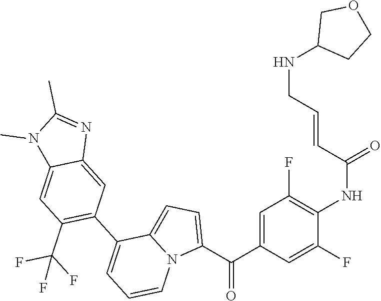 [SMILES: Cc1nc2cc(-c3cccn4c(C(=O)c5cc(F)c(NC(=O)/C=C/CNC6CCOC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4- ((tetrahydrofuran-3- yl)amino)but-2-enamide | 1H-NMR (400 MHz, CHLOROFORM-d) δ 9.97 (d, J = 7.0 Hz, 1H), 7.78 (s, 1H), 7.69 (s, 1H), 7.47-7.44 (m, 2H), 7.31 (d, J = 5.0 Hz, 1H), 7.19 (d, J = 7.0 Hz, 1H), 7.14-7.05 (m, 2H), 6.91 (s, 1H), 6.26 (d, J = 15.3 Hz, 1H), 6.10 (d, J = 4.8 Hz, 1H), 3.95 (q, J = 7.7 Hz, 1H), 3.87 (s, 3H), 3.81 (ddd, J = 16.8, 8.7, 5.8 Hz, 1H), 3.68-3.61 (m, 2H), 3.49-3.44 (m, 3H), 2.70 (s, 3H), 2.18- 2.09 (m, 1H), 1.79-1.72 (m, 1H) | 638.3 |
| 78 | 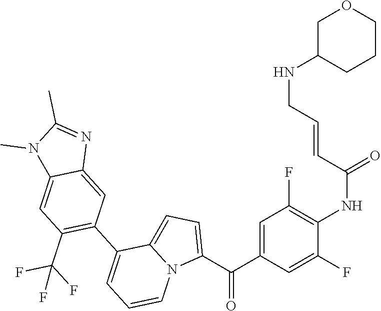 [SMILES: Cc1nc2cc(-c3cccn4c(C(=O)c5cc(F)c(NC(=O)/C=C/CNC6CCCOC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((tetrahydro- 2H-pyran-3-yl)amino)but-2- enamide | 1H-NMR (400 MHz, CHLOROFORM-d) δ 9.97 (d, J = 7.0 Hz, 1H), 7.78 (s, 1H), 7.69 (s, 1H), 7.47-7.44 (m, 2H), 7.32-7.29 (m, 1H), 7.21-7.18 (m, 1H), 7.13-7.05 (m, 2H), 6.92 (s 1H), 6.26 (d, J = 15.3 Hz, 1H), 6.10 (d, J = 5.5 Hz, 1H), 3.88 (s, 3H), 3.78 (dt, J = 11.3, 4.4 Hz, 1H), 3.68-3.62 (m, 1H), 3.53-3.45 (m, 3H), 3.26 (dd, J = 11.0, 7.8 Hz, 1H), 2.74-2.70 (m, 4H), 2.07-1.96 (m, 2H), 1.75-1.73 (m, 2H) | 652.3 |
| 79 | 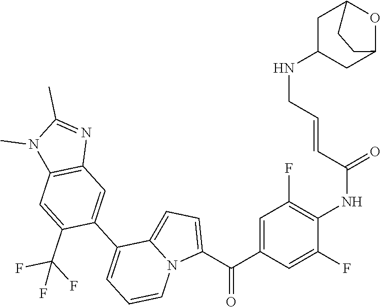 [SMILES: Cc1nc2cc(-c3cccn4c(C(=O)c5cc(F)c(NC(=O)/C=C/CNC6CC7CCC(C6)O7)c(F)c5)ccc34)c(C(F)(F)F)cc2n1C] | (E)-4-((8- oxabicyclo[3.2.1]octan-3- yl)amino)-N-(4-(8-(1,2- dimethyl-6-(trifluoromethyl)- 1H-benzo[d]imidazol-5- yl)indolizine-3-carbonyl)-2,6- difluorophenyl)but-2-enamide | 1H-NMR (400 MHz, CHLOROFORM-d) δ 9.97 (d, J = 7.0 Hz, 1H), 7.78 (s, 1H), 7.69 (s, 1H), 7.48-7.44 (m, 2H), 7.32-7.31 (m, 1H), 7.21-7.06 (m, 3H), 6.88 (s, 1H), 6.22 (d, J = 15.0 Hz, 1H), 6.10 (d, J = 5.0 Hz, 1H), 4.38-4.32 (m, 2H), 3.87 (s, 3H), 3.65 (t, J = 6.1 Hz, 1H), 3.45-3.44 (m, 2H), 3.03-2.99 (m, 1H), 2.70 (s, 3H), 2.18-2.03 (m, 4H), 1.95-1.90 (m, 2H), 1.60-1.52 (m, 2H) | 678.3 |
| 80 | 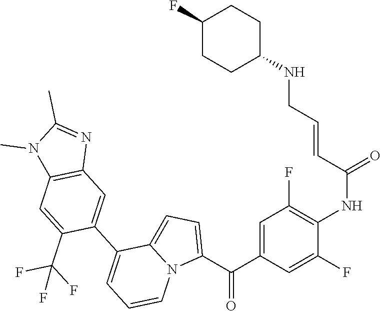 [SMILES: Cc1nc2cc(-c3cccn4c(C(=O)c5cc(F)c(NC(=O)/C=C/CN[C@H]6CC[C@H](F)CC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-(((1r,4r)-4- fluorocyclohexyl)amino)but-2- enamide | 1H-NMR (400 MHz, CHLOROFORM-d) δ 9.97 (dt, J = 7.0, 0.9 Hz, 1H), 7.77 (s, 1H), 7.68 (s, 1H), 7.47-7.42 (m, 2H), 7.30 (d, J = 4.8 Hz, 1H), 7.19 (d, J = 7.0 Hz, 1H), 7.14-7.05 (m, 2H), 6.98 (s, 1H), 6.23 (dt, J = 15.3, 1.8 Hz, 1H), 6.10 (dd, J = 4.9, 0.6 Hz, 1H), 1H), 4.64-4.45 (m, 1H), 3.86 (s, 3H), 3.49 (dd, J = 5.3, 1.8 Hz, 2H), 2.70 (s, 3H), 2.63-2.57 (m, 1H), 2.15-1.95 (m, 4H), 1.62-1.50 (m, 2H), 1.25-1.15 (m, 2H) | 668.4 |
| 81 | 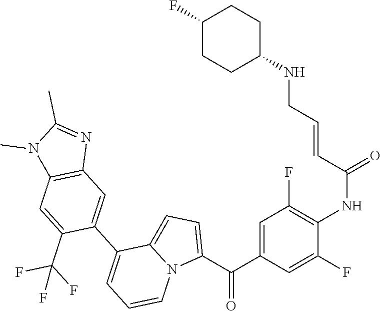 [SMILES: Cc1nc2cc(-c3cccn4c(C(=O)c5cc(F)c(NC(=O)/C=C/CN[C@H]6CC[C@@H](F)CC6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-(((1s,4s)-4- fluorocyclohexyl)amino)but-2- enamide | 1H-NMR (400 MHz, CHLOROFORM-d) δ 9.97 (dt, J = 7.2, 0.9 Hz, 1H), 7.77 (s, 1H), 7.68 (s, 1H), 7.47-7.41 (m, 2H), 7.31 (d, J = 4.8 Hz, 1H), 7.19 (d, J = 6.8 Hz, 1H), 7.15-7.05 (m, 3H), 6.27 (dt, J = 15.3, 1.8 Hz, 1H), 6.10 (dd, J = 4.8, 0.5 Hz, 1H), 4.86-4.68 (m, 1H), 3.86 (s, 3H), 3.53 (dd, J = 5.1, 1.9 Hz, 2H), 2.70 (s, 3H), 2.62-2.53 (m, 1H), 2.11-1.99 (m, 2H), 1.79-1.71 (m, 2H), 1.64-1.45 (m, 4H) | 668.4 |
| 82 | 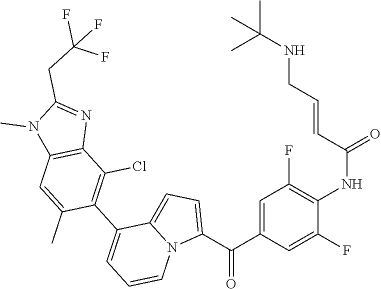 [SMILES: Cc1cc2c(nc(CC(F)(F)F)n2C)c(Cl)c1-c1cccn2c(C(=O)c3cc(F)c(NC(=O)/C=C/CNC(C)(C)C)c(F)c3)ccc12] | (E)-4-(tert-butylamino)-N-(4- (8-(4-chloro-1,6-dimethyl-2- (2,2,2-trifluoroethyl)-1H- benzo[d]imidazol-5- yl)indolizine-3-carbonyl)-2,6- difluorophenyl)but-2-enamide | 1H-NMR (400 MHz, CHLOROFORM-d) δ 9.98 (dd, J = 6.5, 0.8 Hz, 1H), 7.44 (d, J = 7.8 Hz, 2H), 7.33 (d, J = 4.8 Hz, 1H), 7.16-7.08 (m, 3H), 6.41 (d, J = 15.3 Hz, 1H), 6.07 (dd, J = 4.9, 0.6 Hz, 1H), 3.91 (q, J = 9.9 Hz, 2H), 3.87 (s, 3H), 3.51 (dd, J = 5.8, 1.3 Hz, 2H), 2.20 (s, 3H), 1.23 (s, 9H) | 672.3 |
| 83-1, 83-2 | 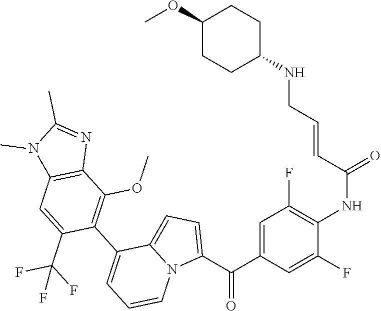 [SMILES: COc1c(-c2cccn3c(C(=O)c4cc(F)c(NC(=O)/C=C/CN[C@H]5CC[C@H](OC)CC5)c(F)c4)ccc23)c(C(F)(F)F)cc2c1nc(C)n2C] | (E)-N-(2,6-difluoro-4-(8-(4- methoxy-1,2-dimethyl-6- (trifluoromethyl)-1H- benzo[d]imidazol-5- yl)imidazo[1,2-a]pyridine-3- carbonyl)phenyl)-4-(((1r,4r)-4- methoxycyclohexyl)amino)but- 2-enamide (1^{st} eluting isomer and 2^{nd} eluting isomer) | 1H-NMR (400 MHz, CHLOROFORM-d) δ 9.73 (d, J = 6.8 Hz, 1H), 8.17 (s, 1H), 7.53-7.48 (m, 3H), 7.44 (s, 1H), 7.08 (td, J = 10.3, 5.1 Hz, 1H), 6.20 (d, J = 15.3 Hz, 1H), 4.14 (s, 3H), 3.80 (s, 3H), 3.48 (d, J = 5.3 Hz, 2H), 3.36 (s, 3H), 3.18- 3.10 (m, 1H), 2.64 (s, 3H), 2.56-2.48 (m, 1H), 2.18 (d, J = 2.5 Hz, 1H), 2.10-1.96 (m, 4H), 1.27- 1.13 (m, 4H) | 711.3 |
| 84 | 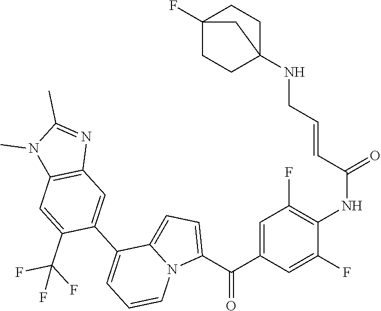 [SMILES: Cc1nc2cc(-c3cccn4c(C(=O)c5cc(F)c(NC(=O)/C=C/CNC67CCC(F)(CC6)C7)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- diflurophenyl)-4-((4- fluorobicyclo[2.2.1]heptan-1- yl)amino)but-2-enamide | 1H-NMR (400 MHz, CHLOROFORM-D) δ 9.97 (ddd, J = 7.2, 0.8, 0.8 Hz, 1H), 7.77 (s, 1H), 7.68 (s, 1H), 7.47-7.40 (m, 2H), 7.30 (d, J = 4.8 Hz, 1H), 7.19 (ddd, J = 6.8, 0.8, 0.8 Hz, 1H), 7.15- 7.00 (m, 3H), 6.26 (dt, J = 15.3, 1.9 Hz, 1H), 6.10 (dd, J = 4.9, 0.6 Hz, 1H), 3.86 (s, 3H), 3.49 (dd, J = 5.0, 2.0 Hz, 2H), 2.70 (s, 3H), 2.05-1.93 (m, 2H), 1.89-1.80 (m, 4H), 1.77 (s, 2H), 1.71-1.66 (m, 2H) | 680.4 |
| 85 | 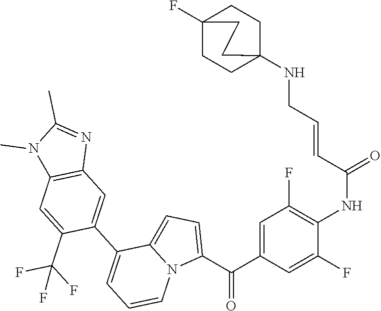 [SMILES: Cc1nc2cc(-c3cccn4c(C(=O)c5cc(F)c(NC(=O)/C=C/CNC67CCC(F)(CC6)CC7)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-((4- fluorobicyclo[2.2.2]octan-1- yl)amino)but-2-enamide | 1H-NMR (400 MHz, CHLOROFORM-D) δ 9.97 (ddd, J = 7.2, 0.8, 0.8 Hz, 1H), 7.77 (s, 1H), 7.68 (s, 1H), 7.47-7.41 (m, 2H), 7.30 (d, J = 5.0 Hz, 1H), 7.19 (ddd, J = 7.2, 0.8, 0.8 Hz, 1H), 7.12- 7.05 (m, 2H), 6.94 (s, 1H), 6.23 (dt, J = 15.3, 1.8 Hz, 1H), 6.10 (dd, J = 4.8, 0.8 Hz, 1H), 3.86 (s, 3H), 3.38 (dd, J = 5.1, 1.9 Hz, 2H), 2.70 (s, 3H), 1.95-1.87 (m, 6H), 1.80-1.73 (m, 6H) | 694.4 |
| 86 | 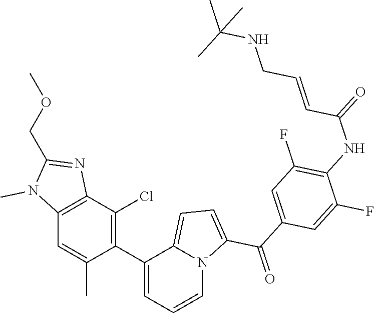 [SMILES: COCc1nc2c(Cl)c(-c3cccn4c(C(=O)c5cc(F)c(NC(=O)/C=C/CNC(C)(C)C)c(F)c5)ccc34)c(C)cc2n1C] | (E)-4-(tert-butylamino)-N-(4- (8-(4-chloro-2- (methoxymethyl)-1,6-dimethyl- 1H-benzo[d]imidazol-5- yl)indolizine-3-carobnyl)-2,6- difluorophenyl)but-2-enamide | 1H-NMR (400 MHz, CHLOROFORM-d) δ 9.99 (dd, J = 6.8, 0.7 Hz, 1H), 7.48-7.44 (m, 2H), 7.33 (d, J = 5.0 Hz, 1H), 7.25 (d, J = 0.8 Hz, 1H), 7.18-7.10 (m, 3H), 6.29 (d, J = 15.3 Hz, 1H), 6.06 (dd, J = 4.8, 0.8 Hz, 1H), 4.84 (s, 2H), 3.91 (s, 3H), 3.48-3.46 (m, 2H), 3.44 (s, 3H), 2.20 (s, 3H), 1.17 (s, 9H) | 634.3 |
| 87 | 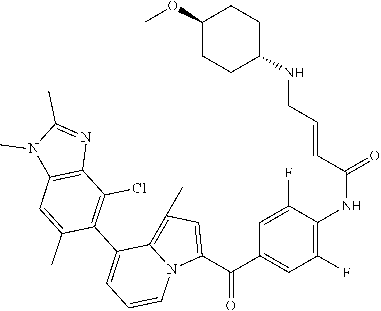 [SMILES: CO[C@H]1CC[C@H](NC/C=C/C(=O)Nc2c(F)cc(C(=O)c3cc(C)c4c(-c5c(C)cc6c(nc(C)n6C)c5Cl)cccn34)cc2F)CC1] | (E)-N-(4-(8-(4-chloro-1,2,6- trimethyl-1H- benzo[d]imidazol-5-yl)-1- methylindolizine-3-carbonyl)- 2,6-difluorophenyl)-4-(((1r,4r)- 4- methoxycyclohexyl)amino)but- 2-enamide | 1H-NMR (400 MHz, DMSO-d6) δ 9.92-9.85 (m, 2H), 7.46-7.41 (m, 3H), 7.18-7.04 (m, 3H), 6.80 (d, J = 15.3 Hz, 1H), 6.26 (d, J = 15.3 Hz, 1H), 3.70 (s, 3H), 3.14 (s, 3H), 3.00 (m, 2H), 2.43 (m, 2H), 2.30 (brs, 1H), 2.01 (s, 3H), 1.84 (m, 4H), 1.39 (s, 3H), 1.15-0.95 (m, 4H) | 674.0 |
| 88 | 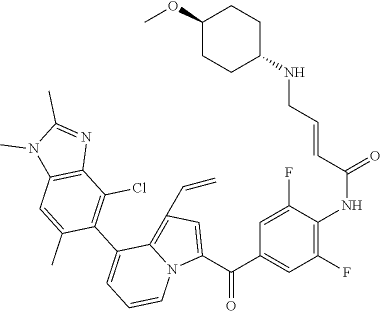 [SMILES: C=Cc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro-1,2,6- trimethyl-1H- benzo[d]imidazol-5-yl)-1- vinylindolizine-3-carbonyl)- 2,6-difluorophenyl)-4-(((1r,4r)- 4- methoxycyclohexyl)amino)but- 2-enamide | 1H-NMR (400 MHz, DMSO-d6) δ 10.01 (s, 1H), 9.87 (d, J = 6.5 Hz, 1H), 7.64 (s, 1H), 7.57 (d, J = 8.0 Hz, 2H), 7.51 (s, 1H), 7.26 (t, J = 7.0 Hz, 1H), 7.15 (d, J = 6.3 Hz, 1H), 6.86 (td, J = 10.3, 5.2 Hz, 1H), 6.33 (d, J = 15.5 Hz, 1H), 5.52-5.38 (m, 2H), 4.59 (d, J = 12.3 Hz, 1H), 3.78 (s, 3H), 3.27- 3.19 (m, 5H), 3.09-3.01 (m, 1H), 2.57 (s, 3H), 2.40-2.34 (m, 1H), 2.06 (s, 3H), 1.96-1.86 (m, 4H), 1.18-0.98 (m, 4H) | 686.0 |
| 89 | 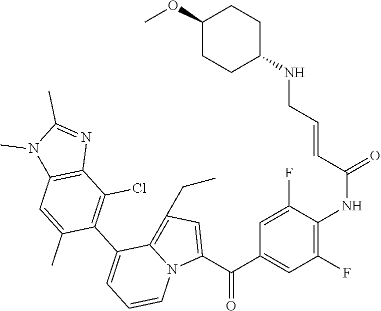 [SMILES: CCc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro-1,2,6- trimethyl-1H- benzo[d]imidazol-5-yl)-1- ethylindolizine-3-carbonyl)- 2,6-difluorophenyl)-4-(((1r,4r)- 4- methoxycyclohexyl)amino)but- 2-enamide | 1H-NMR (400 MHz, DMSO-d6) δ 9.99 (s, 1H), 9.95-9.93 (dd, J = 6.8, 0.8 Hz, 1H), 7.57-7.50 (m, 1H), 7.53 (d, J = 8.0 Hz, 1H), 7.48 (s, 1H), 7.23 (t, J = 7.0 Hz, 1H), 7.23 (s, 1H), 7.08 (dd, J = 7.0, 1.0 Hz, 1H), 6.86 (td, J = 10.3, 5.2 Hz, 1H), 6.32 (d, J = 15.5 Hz, 1H), 3.77 (s, 3H), 3.20 (s, 3H), 3.09-3.03 (m, 1H), 2.56 (s, 3H), 2.40-2.35 (m, 1H), 2.08 (s, 4H), 1.96-1.86 (m, 4H), 1.81 (q, J = 7.5 Hz, 2H), 1.15-0.98 (m, 4H), 0.79 (t, J = 7.5 Hz, 3H) | 687.9 |
| 90 | 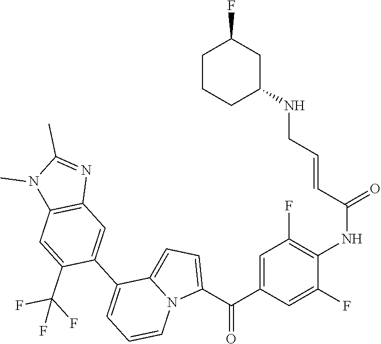 [SMILES: Cc1nc2cc(-c3cccn4c(C(=O)c5cc(F)c(NC(=O)/C=C/CN[C@@H]6CCC[C@@H](F)C6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-(((1R,3R)-3- fluorocyclohexyl)amino)but-2- enamide and (E)-N-(4-(8-(1,2- dimethyl-6-(trifluromethyl)- 1H-benzo[d]imidazol-5- yl)indolizine-3-carbonyl)-2,6- difluorophenyl)-4-(((1S,3S)-3- fluorocylcohexyl)amino)but-2- enamide | 1H-NMR (400 MHz, CHLOROFORM-d) δ 9.97 (d, J = 7.1 Hz, 1H), 7.78 (s, 1H), 7.69 (s, 1H), 7.45 (d, J = 8.0 Hz, 2H), 7.31 (d, J = 4.8 Hz, 1H), 7.19 (d, J = 7.0 Hz, 1H), 7.20-7.00 (m, 3H), 6.26 (d, J = 15.4 Hz, 1H), 6.10 (d, J = 4.8 Hz, 1H), 5.30-4.80 (m, 1H), 3.87 (s, 3H), 3.52 (d, J = 5.09 Hz, 2H), 3.00-2.90 (m, 1H), 2.70 (s, 3H), 2.30- 2.15 (m, 1H), 2.00-1.90 (m, 2H), 1.80-1.20 (m, 5H) | 668.4 |
| 91 | 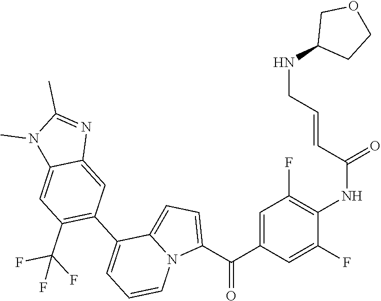 [SMILES: Cc1nc2cc(-c3cccn4c(C(=O)c5cc(F)c(NC(=O)/C=C/CN[C@@H]6CCOC6)c(F)c5)ccc34)c(C(F)(F)F)cc2n1C] | (R,E)-N-(4-(8-(1,2-dimethyl-6- (trifluoromethyl)-1H- benzo[d]imidazol-5- yl)indolizine-3-carbonyl)-2,6- difluorophenyl)-4- ((tetrahydrofuran-3- yl)amino)but-2-enamide | 1H-NMR (400 MHz, CHLOROFORM-d) δ 9.97 (d, J = 7.0 Hz, 1H), 7.78 (s, 1H), 7.69 (s, 1H), 7.47-7.44 (m, 2H), 7.31 (d, J = 5.0 Hz, 1H), 7.19 (d, J = 7.0 Hz, 1H), 7.14-7.05 (m, 2H), 6.91 (s, 1H), 6.26 (d, J = 15.3 Hz, 1H), 6.10 (d, J = 4.8 Hz, 1H), 3.95 (q, J = 7.7 Hz, 1H), 3.87 (s, 3H), 3.81 (ddd, J = 16.8, 8.7, 5.8 Hz, 1H), 3.68-3.61 (m, 2H), 3.49-3.44 (m, 3H), 2.70 (s, 3H), 2.18- 2.09 (m, 1H), 1.79-1.72 (m, 1H) | 638.3 |
| 92 | 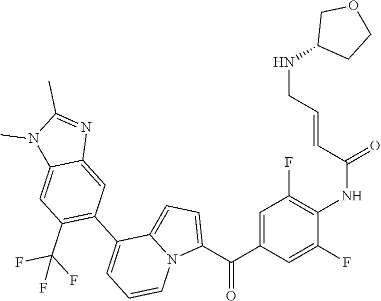 [SMILES: Cc1nc2cc(-c3cccn4c(C(=O)c5cc(F)c(NC(=O)/C=C/CN[C@H]6CCOC6)c(F)c5)ccc34)c(C(F)(F)F)cc2n1C] | (S,E)-N-(4-(8-(1,2-dimethyl-6- (trifluoromethyl)-1H- benzo[d]imidazol-5- yl)indolizine-3-carbonyl)-2,6- difluorophenyl)-4- ((tetrahydrofuran-3- yl)amino)but-2-enamide | 1H-NMR (400 MHz, CHLOROFORM-d) δ 9.97 (d, J = 7.0 Hz, 1H), 7.78 (s, 1H), 7.69 (s, 1H), 7.47-7.44 (m, 2H), 7.31 (d, J = 5.0 Hz, 1H), 7.19 (d, J = 7.0 Hz, 1H), 7.14-7.05 (m, 2H), 6.91 (s, 1H), 6.26 (d, J = 15.3 Hz, 1H), 6.10 (d, J = 4.8 Hz, 1H), 3.95 (q, J = 7.7 Hz, 1H), 3.87 (s, 3H), 3.81 (ddd, J = 16.8, 8.7, 5.8 Hz, 1H), 3.68-3.61 (m, 2H), 3.49-3.44 (m, 3H), 2.70 (s, 3H), 2.18- 2.09 (m, 1H), 1.79-1.72 (m, 1H) | 638.3 |
| 93 | 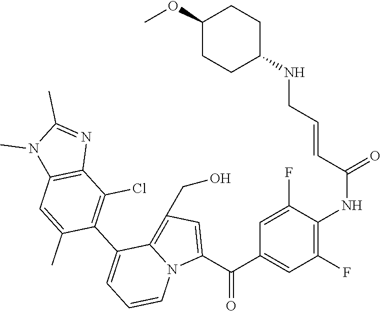 [SMILES: CO[C@H]1CC[C@H](NC/C=C/C(=O)Nc2c(F)cc(C(=O)c3cc(CO)c4c(-c5c(C)cc6c(nc(C)n6C)c5Cl)cccn34)cc2F)CC1] | (E)-N-(4-(8-(4-chloro-1,2,6- trimethyl-1H- benzo[d]imidazol-5-yl)-1- (hydroxymethyl)indolizine-3- carobnyl)-2,6-difluorophenyl)- 4-(((1r,4r)-4- methoxycyclohexyl)amino)but- 2-enamide | 1H-NMR (400 MHz, CHLOROFORM-D) δ 10.02 (dd, J = 5.8, 2.3 Hz, 1H), 7.42 (d, J = 8.0 Hz, 2H), 7.36 (s, 1H), 7.15 (s, 1H), 7.11-7.04 (m, 3H), 6.21 (d, J = 15.3 Hz, 1H), 3.93 (q, J = 15.2 Hz, 2H), 3.75 (s, 3H), 3.47 (dd, J = 5.3, 1.5 Hz, 2H), 3.33 (s, 3H), 3.15-3.08 (m, 1H), 2.64 (s, 3H), 2.55-2.47 (m, 1H), 2.15 (s, 3H), 2.07-1.94 (m, 4H), 1.27-1.07 (m, 6H) | 690.0 |
| 94 | 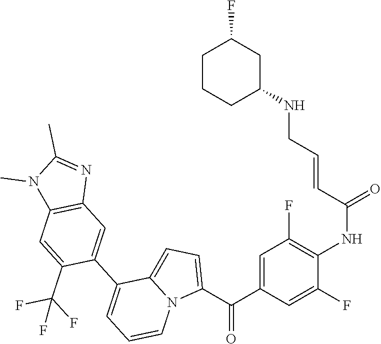 [SMILES: Cc1nc2cc(-c3cccn4c(C(=O)c5cc(F)c(NC(=O)/C=C/CN[C@@H]6CCC[C@H](F)C6)c(F)c5)ccc34)c(C(F)(F)F)cc2n1C] | (E)-N-(4-(8-(1,2-dimethyl-6- (trifluoromethyl)-1H- benzo[d]imidazol-5- yl)indolizine-3-carbonyl)-2,6- difluorophenyl)-4-(((1R,3S)-3- fluorocyclohexyl)amino)but-2- enamide and (E)-N-(4-(8-(1,2- dimethyl-6-(trifluoromethyl)- 1H-benzo[d]imidazol-5- yl)indolizine-3-carbonyl)-2,6- difluorophenyl)-4-(((1S,3R)-3- fluorocyclohexyl)amino)but-2- enamide | 1H-NMR (400 MHz, CHLOROFORM-d) δ 9.97 (d, J = 7.0 Hz, 1H), 7.78 (s, 1H), 7.69 (s, 1H), 7.45 (d, J = 7.9 Hz, 2H), 7.31 (d, J = 4.8 Hz, 1H), 7.20 (d, J = 6.9 Hz, 1H), 7.15-7.00 (m, 3H), 6.26 (d, J = 15.4 Hz, 1H), 6.10 (d, J = 4.8 Hz, 1H), 4.65-4.40 (m, 1H), 3.87 (s, 3H), 3.55-3.50 (m, 2H), 2.70 (s, 3H), 2.65-2.55 (m, 1H), 2.40-2.30 (m, 1H), 2.15-2.05 (m, 1H), 1.95-1.80 (m, 2H), 1.50-1.20 (m, 3H), 1.15-1.00 (m, 1H) | 668.3 |
| 95 | 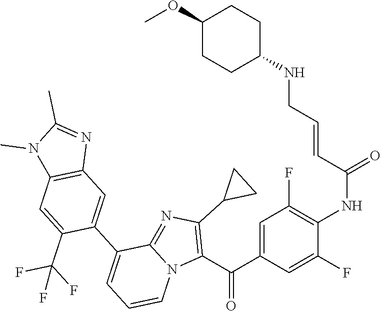 [SMILES: CO[C@H]1CC[C@H](NC/C=C/C(=O)Nc2c(F)cc(C(=O)c3c(C4CC4)nc4c(-c5cc6nc(C)n(C)c6cc5C(F)(F)F)cccn34)cc2F)CC1] | (E)-N-(4-(2-cyclopropyl-8-(1,2- dimethyl-6-(trifluoromethyl)- 1H-benzo[d]imidazol-5- yl)imidazo[1,2-a]pyridine-3- carbonyl)-2,6-difluorophenyl)- 4-(((1r,4r)-4- methoxycyclohexyl)amino)but- 2-enamide | 1H-NMR (400 MHz, CHLOROFORM-D) δ 9.51 (d, J = 6.8 Hz, 1H), 7.73 (s, 1H), 7.69 (s, 1H), 7.48 (d, J = 8.0 Hz, 2H), 7.41 (d, J = 7.0 Hz, 1H), 7.14-7.06 (m, 3H), 6.23 (d, J = 15.3 Hz, 1H), 3.84 (s, 3H), 3.51 (d, J = 5.3 Hz, 2H), 3.35 (s, 3H), 3.17-3.11 (m, 1H), 2.68 (s, 3H), 2.56-2.50 (m, 1H), 2.09-1.97 (m, 4H), 1.30-1.10 (m, 4H), 1.05- 1.01 (m, 2H), 0.7-0.74 (m, 2H) | 721.4 |
| 96 | 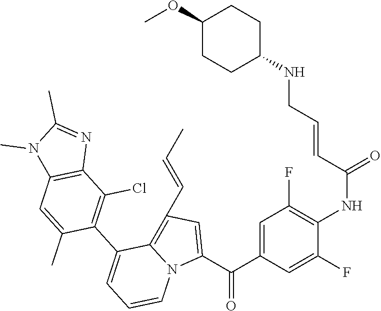 [SMILES: C/C=C/c1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro-1,2,6- trimethyl-1H- benzo[d]imidazol-5-yl)-1-((E)- prop-1-en-1-yl)indolizine-3- carbonyl)-2,6-difluorophenyl)- 4-(((1r,4r)-4- methoxycyclohexyl)amino)but- 2-enamide | 1H-NMR (400 MHz, CHLOROFORM-D) δ 9.99-9.95 (m, 1H), 7.45 (d, J = 7.8 Hz, 2H), 7.37 (s, 1H), 7.15-7.02 (m, 4H), 6.26 (d, J = 15.5 Hz, 1H), 5.80 (dt, J = 15.5, 6.6 Hz, 1H), 5.18 (d, J = 1 5.5 Hz, 1H), 3.79 (s, 3H), 3.52 (d, J = 5.3 Hz, 2H), 3.35 (s, 3H), 3.18-3.11 (m, 1H), 2.68 (s, 3H), 2.57-2.51 (m, 1H), 2.13 (s, 3H), 2.10-1.98 (m, 4H), 1.36-1.11 (m, 8H) | 699.5 |
| 97 | 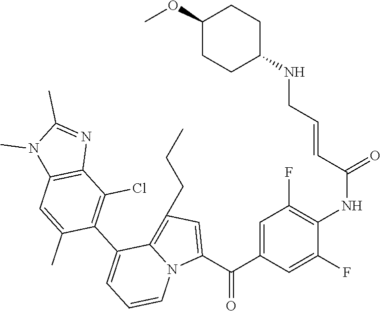 [SMILES: CCCc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro-1,2,6- trimethyl-1H- benzo[d]imidazol-5-yl)-1- propylindolizine-3-carbonyl)- 2,6-difluorophenyl)-4-(((1r,4r)- 4- methoxycyclohexyl)amino)but- 2-enamide | 1H-NMR (400 MHz, CHLOROFORM-D) δ 10.05 (dd, J = 6.8, 1.3 Hz, 1H), 7.55-7.36 (m, 3H), 7.15-7.14 (m, 2H), 7.12-7.00 (m, 2H), 6.29 (d, J = 15.5 Hz, 1H), 3.78 (s, 3H), 3.51 (dd, J = 5.1, 1.4 Hz, 2H), 3.34 (s, 3H), 3.18-3.11 (m, 1H), 2.68 (s, 3H), 2.58-2.51 (m, 1H), 2.16 (s, 3H), 2.13-1.97 (m, 4H), 1.85 (t, J = 7.4 Hz, 2H), 1.32- 1.10 (m, 6H), 0.51 (t, J = 7.4 Hz, 3H) | 701.9 |
| 98 | 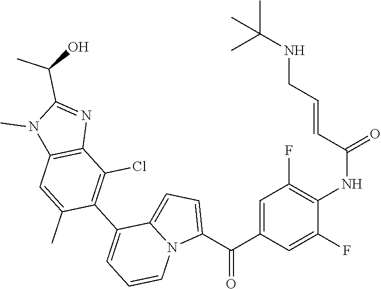 [SMILES: Cc1cc2c(nc([C@@H](C)O)n2C)c(Cl)c1-c1cccn2c(C(=O)c3cc(F)c(NC(=O)/C=C/CNC(C)(C)C)c(F)c3)ccc12] | (R,E)-4-(tert-butylamino)-N-(4- (8-(4-chloro-2-(1- hydroxyethyl)-1,6-dimethyl- 1H-benzo[d]imidazol-5- yl)indolizine-3-carbonyl)-2,6- difluorophenyl)but-2-enamide | 1H-NMR (400 MHz, CHLOROFORM-d) δ 9.98-9.97 (m, 1H), 7.40-7.50 (m, 2H), 7.30-7.26 (m, 1H), 7.25-7.20 (m, 2H), 7.19-7.10 (m, 3H), 6.40-6.30 (m, 1H), 6.08-6.00 (m, 1H), 5.31-5.20 (m, 1H), 3.93 (s, 1.5H), 3.90 (s, 1.5H), 3.46 (d, J = 4.1 Hz, 2H), 2.20-2.15 (brs, 3H), 1.80-1.75 (brs, 3H), 1.17 (s, 9H) | 634.4 |
| 99 | 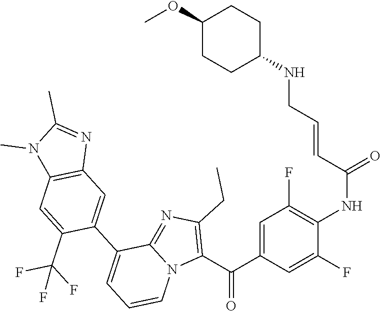 [SMILES: CCc1nc2c(-c3cc4nc(C)n(C)c4cc3C(F)(F)F)cccn2c1C(=O)c1cc(F)c(NC(=O)/C=C/CN[C@H]2CC[C@H](OC)CC2)c(F)c1] | (E)-N-(4-(8-(1,2-dimethyl-6- (trifluoromethyl)-1H- benzo[d]imidazol-5-yl)-2- ethylimidazo[1,2-a]pyridine-3- carbonyl)-2,6-difluorophenyl)- 4-(((1r,4r)-4- methoxycyclohexyl)amino)but- 2-enamide | 1H-NMR (400 MHz, CHLOROFORM-D) δ 9.45 (dd, J = 7.0, 1.3 Hz, 1H), 7.73 (d, J = 6.8 Hz, 2H), 7.44 (d, J = 7.3 Hz, 1H), 7.35 (d, J = 7.8 Hz, 2H), 7.14-7.06 (m, 2H), 6.23 (dt, J = 15.3, 1.6 Hz, 1H), 3.83 (s, 3H), 3.49 (dd, J = 5.3, 1.8 Hz, 2H), 3.35 (s, 3H), 3.17-3.12 (m, 1H), 2.67 (s, 3H), 2.54-2.44 (m, 3H), 2.09-1.96 (m, 4H), 1.26-1.13 (m, 4H), 1.04 (t, J = 7.5 Hz, 3H) | 709.4 |
| 100 | 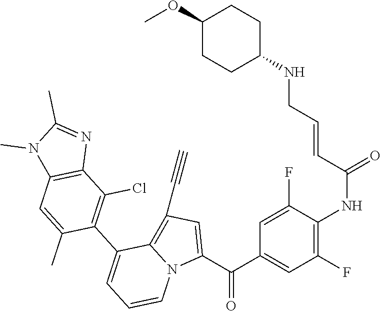 [SMILES: C#Cc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro-1,2,6- trimethyl-1H- benzo[d]imidazol-5-yl)-1- ethynylindolizine-3-carbonyl)- 2,6-difluorophenyl)-4-(((1r,4r)- 4- methoxycyclohexyl)amino)but- 2-enamide | 1H-NMR (400 MHz, CHLOROFORM-D) δ 9.98 (d, J = 6.8 Hz, 1H), 7.47-7.43 (m, 4H), 7.20-7.08 (m, 3H), 6.27 (d, J = 15.5 Hz, 1H), 3.77 (s, 3H), 3.50 (d, J = 4.8 Hz, 2H), 3.35 (s, 3H), 3.18-3.11 (m, 1H), 2.66 (s, 3H), 2.57-2.52 (m, 1H), 2.25 (s, 1H), 2.16 (s, 3H), 2.12-1.97 (m, 4H), 1.30-1.11 (m, 4H) | 684.0 |
| 101 | 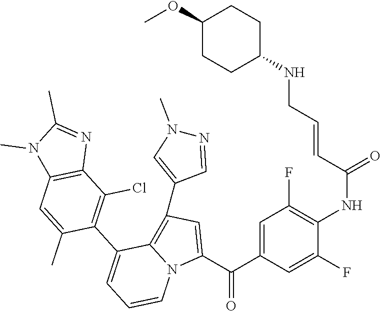 [SMILES: CO[C@H]1CC[C@H](NC/C=C/C(=O)Nc2c(F)cc(C(=O)c3cc(-c4cnn(C)c4)c4c(-c5c(C)cc6c(nc(C)n6C)c5Cl)cccn34)cc2F)CC1] | (E)-N-(4-(8-(4-chloro-1,2,6- trimethyl-1H- benzo[d]imidazol-5-yl)-1-(1- methyl-1H-pyrazol-4- yl)indolizine-3-carbonyl)-2,6- difluorophenyl)-4-(((1r,4r)-4- methoxycyclohexyl)amino)but- 2-enamide | 1H-NMR (400 MHz, CHLOROFORM-D) δ 10.08 (dd, J = 5.8, 2.5 Hz, 1H), 7.46 (d, J = 7.8 Hz, 2H), 7.25 (s, 1H), 7.15-7.07 (m, 3H), 6.90 (s, 1H), 6.85 (s, 1H), 6.61 (s 1H), 6.23 (d, J = 15.3 Hz, 1H), 3.71 (s, 3H), 3.50 (d, J = 5.0 Hz, 2H), 3.35 (s, 3H), 3.25 (s, 3H), 3.18-3.11 (m, 1H), 2.65 (s, 3H), 2.56-2.49 (m, 1H), 2.09-1.98 (m, 7H), 1.30-1.09 (m, 4H) | 740.0 |
| 102-1 | 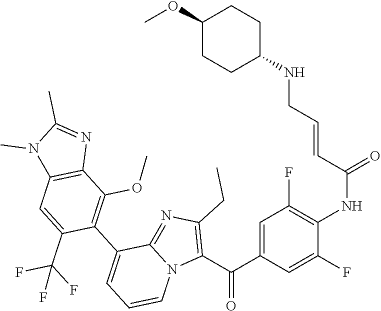 [SMILES: CCc1nc2c(-c3c(C(F)(F)F)cc4c(nc(C)n4C)c3OC)cccn2c1C(=O)c1cc(F)c(NC(=O)/C=C/CN[C@H]2CC[C@H](OC)CC2)c(F)c1] | (E)-N-(4-(2-ethyl-8-(4- methoxy-1,2-dimethyl-6- (trifluoromethyl)-1H- benzo[d]imidazol-5- yl)imidazo[1,2-a]pyridine- 3-carbonyl)-2,6- difluorophenyl)-4- (((1r,4r)-4- methoxycyclohexyl)amino) but-2-enamide | 1H-NMR (400 MHz, CHLOROFORM- D) δ 9.46 (d, J = 6.8 Hz, 1H), 7.44 (s, 1H), 7.37 (dd, J = 12.1, 8.1 Hz, 3H), 71.4-7.07 (m, 2H), 6.23 (d, J = 15.3 Hz, 1H), 4.17 (s, 3H), 3.80 (s, 3H), 3.50 (d, J = 5.0 Hz, 2H), 3.35 (s, 3H), 3.17-3.12 (m, 1H), 2.66 (s, 3H), 2.54-2.40 (m, 3H), 2.11-1.95 (m, 4H), 1.29-1.12 (m, 4H), 1.04 (t, J = 7.4 Hz, 3H) | 739.4 |
| 102-2 | 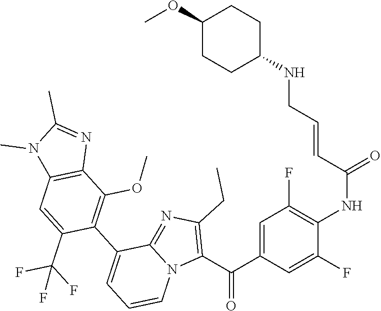 [SMILES: CCc1nc2c(-c3c(C(F)(F)F)cc4c(nc(C)n4C)c3OC)cccn2c1C(=O)c1cc(F)c(NC(=O)/C=C/CN[C@H]2CC[C@H](OC)CC2)c(F)c1] | (E)-N-(4-(2-ethyl-8-(4- methoxy-1,2-dimethyl-6- (trifluoromethyl)-1H- benzo[d]imidazol-5- yl)imidazo[1,2-a]pyridine- 3-carbonyl)-2,6- difluorophenyl)-4- (((1r,4r)-4- methoxycyclohexyl)amino) but-2-enamide | 1H-NMR (400 MHz, CHLOROFORM- D) δ 9.46 (d, J = 6.8 Hz, 1H), 7.44 (s, 1H), 7.37 (dd, J = 12.1, 8.1 Hz, 3H), 7.14-7.07 (m, 2H), 6.23 (d, J = 15.3 Hz, 1H), 4.17 (s, 3H), 3.80 (s, 3H), 3.50 (d, J = 5.0 Hz, 2H), 3.35 (s, 3H), 3.17-3.12 (m, 1H), 2.66 (s, 3H), 2.54-2.40 (m, 3H), 2.11-1.95 (m, 4H), 1.29-1.12 (m, 4H), 1.04 (t, J = 7.4 Hz, 3H) | 739.4 |
| 103 | 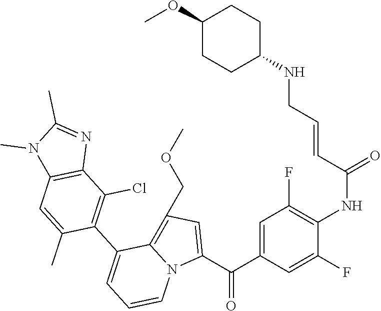 [SMILES: COCc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12] | (E)-N-(4-(8-(4-chloro- 1,2,6-trimethyl-1H- benzo[d]imidazol-5-yl)-1- (methoxymethyl)indolizine- 3-carbonyl)-2,6- difluorophenyl)-4- (((1r,4r)-4- methoxycyclohexyl)amino) but-2-enamide | 1H-NMR (400 MHz, CHLOROFORM- D) δ 10.04 (d, J = 6.3 Hz, 1H), 7.46 (s, 1H), 7.44 (s, 1H), 7.36 (s, 1H), 7.16-7.07 (m, 4H), 6.26 (d, J = 15.3 Hz, 1H), 3.83- 3.66 (m, 5H), 3.51 (d, J = 4.3 Hz, 2H), 3.35 (s, 3H), 3.17-3.12 (m, 1H), 2.81 (s, 3H), 2.68 (s, 3H), 2.58-2.53 (m, 1H), 2.18 (s, 3H), 2.04 (dd, J = 35.0, 11.3 Hz, 4H), 1.30-1.12 (m, 4H) | 704.0 |
| 104 | 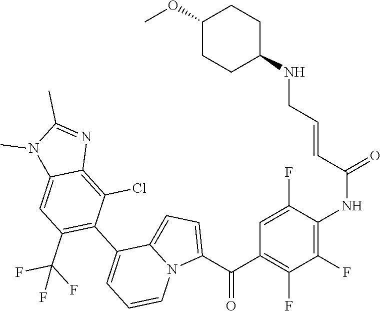 [SMILES: CO[C@H]1CC[C@H](NC/C=C/C(=O)Nc2c(F)cc(C(=O)c3ccc4c(-c5c(C(F)(F)F)cc6c(nc(C)n6C)c5Cl)cccn34)c(F)c2F)CC1] | (E)-N-(4-(8-(4-chloro-1,2- dimethyl-6- (trifluoromethyl)-1H- benzo[d]imidazol-5- yl)indolizine-3-carbonyl)- 2,3,6-trifluorophenyl)-4- (((1r,4r)-4- methoxycyclohexyl)amino) but-2-enamide | 1H-NMR (400 MHz, CHLOROFORM- D) δ 10.04 (d, J = 7.0 Hz, 1H), 7.74 (s, 1H), 7.22-7.17 (m, 3H), 7.16-7.10 (m, 2H), 7.01 (d, J = 6.3 Hz, 1H), 6.24 (dt, J = 15.3, 1.6 Hz, 1H), 5.99 (dd, J = 4.9, 0.6 Hz, 1H), 3.87 (d, J = 4.0 Hz, 3H), 3.51 (dd, J = 5.1, 1.9 Hz, 2H), 3.36-3.35 (m, 3H), 3.17-3.12 (m, 1H), 2.74 (s, 3H), 2.56-2.51 (m, 1H), 2.10-2.06 (m, 2H), 2.01-1.97 (m, 2H), 1.31-1.13 (m, 4H) | 732.1 |
| 105-1 | 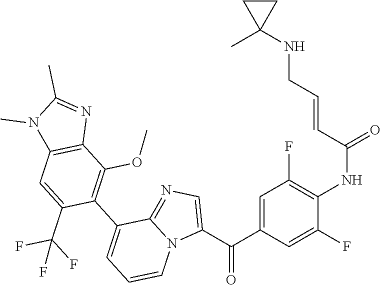 [SMILES: COc1c(-c2cccn3c(C(=O)c4cc(F)c(NC(=O)/C=C/CNC5(C)CC5)c(F)c4)cnc23)c(C(F)(F)F)cc2c1nc(C)n2C] | (E)-N-(2,6-difluoro-4-(8- (4-methoxy-1,2-dimethyl- 6-(trifluoromethyl)-1H- benzo[d]imidazol-5- yl)imidazo[1,2-a]pyridine- 3-carbonyl)phenyl)-4-((1- methylcyclopropyl)amino) but-2-enamide | 1H-NMR (400 MHz, CHLOROFORM- D) δ 9.73 (dd, J = 7.0, 1.3 Hz, 1H), 8.17 (s, 1H), 7.52-7.48 (m, 3H), 7.44 (s, 1H), 7.38-7.36 (brs, 1H), 7.10 (td, J = 10.3, 5.3 Hz, 1H), 6.17 (dt, J = 15.3, 1.8 Hz, 1H), 4.15 (s, 3H), 3.79 (s, 3H), 3.52 (dd, J = 5.0, 1.8 Hz, 2H), 2.65 (s, 3H), 1.27 (s, 3H), 0.58 (t, J = 5.1 Hz, 2H), 0.39 (dd, J = 6.1, 4.4 Hz, 2H) | 653.4 |
| 105-2 | 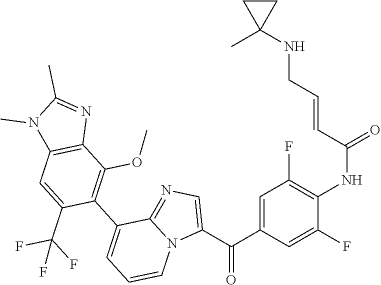 [SMILES: COc1c(-c2cccn3c(C(=O)c4cc(F)c(NC(=O)/C=C/CNC5(C)CC5)c(F)c4)cnc23)c(C(F)(F)F)cc2c1nc(C)n2C] | (E)-N-(2,6-difluoro-4-(8- (4-methoxy-1,2-dimethyl- 6-(trifluoromethyl)-1H- benzo[d]imidazol-5- yl)imidazo[1,2-a]pyridine- 3-carbonyl)phenyl)-4-((1- methylcyclopropyl)amino) but-2-enamide | 1H-NMR (400 MHz, CHLOROFORM- D) δ 9.73 (dd, J = 7.0, 1.3 Hz, 1H), 8.17 (s, 1H), 7.52-7.48 (m, 3H), 7.44 (s, 1H), 7.38-7.36 (brs, 1H), 7.10 (td, J = 10.3, 5.3 Hz, 1H), 6.17 (dt, J = 15.3, 1.8 Hz, 1H), 4.15 (s, 3H), 3.79 (s, 3H), 3.52 (dd, J = 5.0, 1.8 Hz, 2H), 2.65 (s, 3H), 1.27 (s, 3H), 0.58 (t, J = 5.1 Hz, 2H), 0.39 (dd, J = 6.1, 4.4 Hz, 2H) | 653.4 |
| 106 | 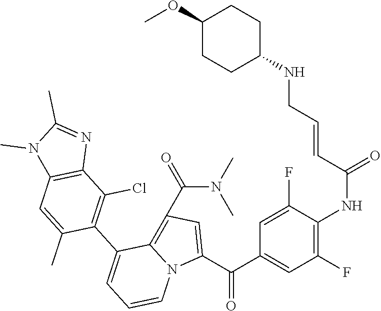 [SMILES: CO[C@H]1CC[C@H](NC/C=C/C(=O)Nc2c(F)cc(C(=O)c3cc(C(=O)N(C)C)c4c(-c5c(C)cc6c(nc(C)n6C)c5Cl)cccn34)cc2F)CC1] | 8-(4-chloro-1,2,6- trimethyl-1H- benzo[d]imidazol-5-yl)-3- (3,5-difluoro-4-((E)-4- (((1r,4r)-4- methoxycyclohexyl)amino) but-2-enamido)benzoyl)- N,N-dimethylindolizine- 1-carboxamide | 1H-NMR (400 MHz, CHLOROFORM- D) δ 10.01 (dd, J = 5.6, 2.6 Hz, 1H), 7.44 (d, J = 8.0 Hz, 2H), 7.29 (s, 1H), 7.19- 7.16 (m, 3H), 7.09 (dt, J = 15.3, 5.4 Hz, 1H), 6.29 (d, J = 15.3 Hz, 1H), 3.76 (s, 3H), 3.52 (d, J = 4.8 Hz, 2H), 3.34 (s, 3H), 3.19-3.11 (m, 1H), 2.76 (s, 3H), 2.65 (s, 3H), 2.61-2.59 (m, 1H), 2.28 (s, 3H), 2.24 (s, 3H), 2.03-194 (m, 4H), 1.33-1.15 (m, 4H) | 731.4 |
| 107 | 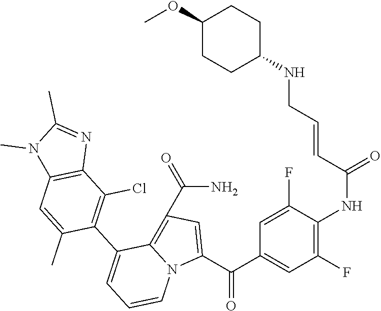 [SMILES: CO[C@H]1CC[C@H](NC/C=C/C(=O)Nc2c(F)cc(C(=O)c3cc(C(N)=O)c4c(-c5c(C)cc6c(nc(C)n6C)c5Cl)cccn34)cc2F)CC1] | 8-(4-chloro-1,2,6- trimethyl-1H- benzo[d]imidazol-5-yl)-3- (3,5-difluoro-4-((E)-4- (((1r,4r)-4- methoxycyclohexyl)amino) but-2- enamido)benzoyl)indolizine- 1-carboxamide | 1H-NMR (400 MHz, CHLOROFORM- D) δ 10.05 (d, J = 6.5 Hz, 1H), 7.64 (s, 1H), 7.46 (s, 1H), 7.44 (s, 1H), 7.27-7.17 (m, 5H), 7.08 (td, J = 10.1, 5.0 Hz, 1H), 6.23 (d, J = 15.3 Hz, 1H), 5.37-4.38 (m, 2H), 3.76 (s, 3H), 3.49 (d, J = 4.3 Hz, 2H), 3.35 (s, 3H), 3.17-3.12 (m, 1H), 2.65 (s, 3H), 2.56-2.51 (m, 1H), 2.20 (s, 3H), 2.09-1.96 (m, 4H), 1.33-1.11 (m, 4H) | 703.3 |
| 108 | 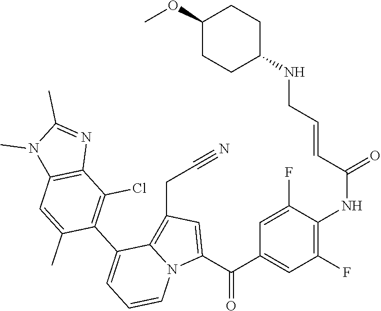 [SMILES: CO[C@H]1CC[C@H](NC/C=C/C(=O)Nc2c(F)cc(C(=O)c3cc(CC#N)c4c(-c5c(C)cc6c(nc(C)n6C)c5Cl)cccn34)cc2F)CC1] | (E)-N-(4-(8-(4-chloro- 1,2,6-trimethyl-1H- benzo[d]imidazol-5-yl)-1- (cyanomethyl)indolizine- 3-carbonyl)-2,6- difluorophenyl)-4- (((1r,4r)-4- methoxycyclohexyl)amino) but-2-enamide | 1H-NMR (400 MHz, CHLOROFORM- D) δ 10.05-10.01 (m, 1H), 7.47-7.42 (m, 2H), 7.37 (s, 1H), 7.24-7.08 (m, 5H), 6.25 (d, J = 15.3 Hz, 1H), 3.80 (s, 3H), 3.51 (dd, J = 5.3, 1.8 Hz, 2H), 3.38-3.33 (m, 3H), 3.21-3.11 (m, 2H), 2.82 (d, J = 18.5 Hz, 1H), 2.69 (s, 3H), 2.58-2.49 (m, 1H), 2.21 (s, 3H), 2.10-1.97 (m, 4H), 1.31-1.10 (m, 4H) | 699.4 |
| 109-1 | 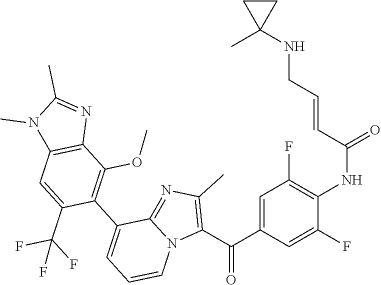 [SMILES: COc1c(-c2cccn3c(C(=O)c4cc(F)c(NC(=O)/C=C/CNC5(C)CC5)c(F)c4)c(C)nc23)c(C(F)(F)F)cc2c1nc(C)n2C] | (E)-N-(2,6-difluoro-4-(8- (4-methoxy-1,2-dimethyl- 6-(trifluoromethyl)-1H- benzo[d]imidazol-5-yl)-2- methylimidazo[1,2- a]pyridine-3- carbonyl)phenyl)-4-((1- methylcyclopropyl)amino) but-2-enamide | 1H-NMR (400 MHz, CHLOROFORM- D) δ 9.51 (dd, J = 6.8, 1.3 Hz, 1H), 7.53 (brs, 1H), 7.41-7.39 (m, 2H), 7.31 (d, J = 8.0 Hz, 2H), 7.14 (t, J = 7.1 Hz, 1H), 7.08 (td, J = 10.3, 5.2 Hz, 1H), 6.17 (dt, J = 15.3, 1.8 Hz, 1H), 4.15 (s, 3H), 3.79 (s, 3H), 3.51 (dd, J = 5.1, 1.6 Hz, 2H), 2.64 (s, 3H), 2.17 (s, 3H), 1.27 (s, 3H), 0.58 (dd, J = 5.9, 4.4 Hz, 2H), 0.39 (dd, J = 6.3, 4.3 Hz, 2H) | 667.5 |
| 109-2 | 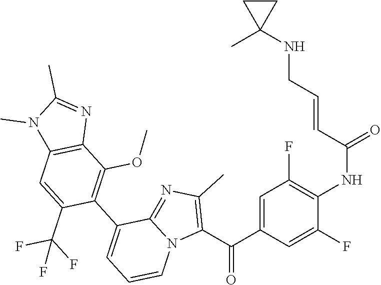 [SMILES: COc1c(-c2cccn3c(C(=O)c4cc(F)c(NC(=O)/C=C/CNC5(C)CC5)c(F)c4)c(C)nc23)c(C(F)(F)F)cc2c1nc(C)n2C] | (E)-N-(2,6-difluoro-4-(8- (4-methoxy-1,2-dimethyl- 6-(trifluoromethyl)-1H- benzo[d]imidazol-5-yl)-2- methylimidazo[1,2- a]pyridine-3- carbonyl)phenyl)-4-((1- methylcyclopropyl)amino) but-2-enamide | 1H-NMR (400 MHz, CHLOROFORM- D) δ 9.51 (dd, J = 6.8, 1.3 Hz, 1H), 7.53 (brs, 1H), 7.41-7.39 (m, 2H), 7.31 (d, J = 8.0 Hz, 2H), 7.14 (t, J = 7.1 Hz, 1H), 7.08 (td, J = 10.3, 5.2 Hz, 1H), 6.17 (dt, J = 15.3, 1.8 Hz, 1H), 4.15 (s, 3H), 3.79 (s, 3H), 3.51 (dd, J = 5.1, 1.6 Hz, 2H), 2.64 (s, 3H), 2.17 (s, 3H), 1.27 (s, 3H), 0.58 (dd, J = 5.9, 4.4 Hz, 2H), 0.39 (dd, J = 6.3, 4.3 Hz, 2H) | 667.5 |
| 110 | 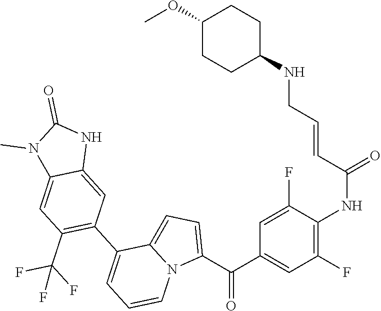 [SMILES: CO[C@H]1CC[C@H](NC/C=C/C(=O)Nc2c(F)cc(C(=O)c3ccc4c(-c5cc6[nH]c(=O)n(C)c6cc5C(F)(F)F)cccn34)cc2F)CC1] | (E)-N-(2,6-difluoro-4-(8- (1-methyl-2-oxo-6- (trifluoromethyl)-2,3- dihydro-1H- benzo[d]imidazol-5- yl)indolizine-3- carobnyl)phenyl)-4- (((1r,4r)-4- methoxycyclohexyl)amino) but-2-enamide | 1H-NMR (400 MHz, CHLOROFORM- D) δ 9.95 (d, J = 7.0 Hz, 1H), 7.44-7.41 (m, 3H), 7.33 (d, J = 4.9 Hz, 1H), 7.16- 7.04 (m, 4H), 6.98 (s, 1H), 6.25 (d, J = 15.3 Hz, 1H), 6.13 (d, J = 5.3 Hz, 1H), 3.52-3.51 (m, 2H), 3.52 (s, 3H), 3.35 (s, 3H), 3.35-3.32 (m, 1H), 3.17-3.12 (m, 1H), 2.55-2.53 (m, 1H), 2.10-2.06 (m, 2H), 1.99 (d, J = 13.3 Hz, 2H), 1.31-1.01 (m, 4H) | 682.3 |
| 111-1 | 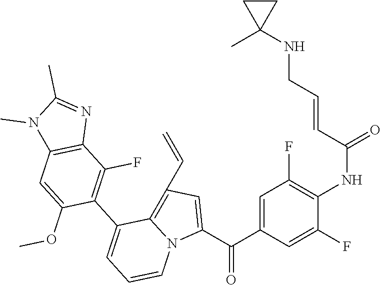 [SMILES: C=Cc1cc(C(=O)c2cc(F)c(NC(=O)/C=C/CNC3(C)CC3)c(F)c2)n2cccc(-c3c(OC)cc4c(nc(C)n4C)c3F)c12] | (E)-N-(2,6-difluoro-4-(8- (4-fluoro-6-methoxy-1,2- dimethyl-1H- benzo[d]imidazol-5-yl)-1- vinylindolizine-3- carobnyl)phenyl)-4-((1- methylcyclopropyl)amino) but-2-enamide | 1H-NMR (400 MHz, CHLOROFORM- D) δ 9.98 (dd, J = 7.0, 1.3 Hz, 1H), 7.46- 7.42 (m, 3H), 7.17-7.10 (m, 2H), 7.06- 7.00 (m, 2H), 6.62 (s, 1H), 6.22 (dt, J = 15.3, 1.9 Hz, 1H), 5.86 (dd, J = 17.4, 10.9 Hz, 1H), 5.34 (dd, J = 17.3, 1.5 Hz, 1H), 4.68 (dd, J = 10.9, 1.4 Hz, 1H), 3.78 (s, 3H), 3.74 (s, 3H), 3.55 (dd, J = 5.0, 1.8 Hz, 2H), 2.64 (s, 3H), 1.29 (s, 3H), 0.62-0.59 (m, 2H), 0.42-0.40 (m, 2H) | 628.3 |
| 111-2 | 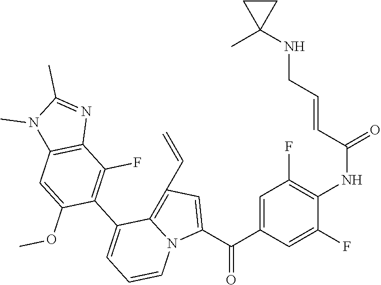 [SMILES: C=Cc1cc(C(=O)c2cc(F)c(NC(=O)/C=C/CNC3(C)CC3)c(F)c2)n2cccc(-c3c(OC)cc4c(nc(C)n4C)c3F)c12] | (E)-N-(2,6-difluoro-4-(8- (4-fluoro-6-methoxy-1,2- dimethyl-1H- benzo[d]imidazol-5-yl)-1- vinylindolizine-3- carbonyl)phenyl)-4-((1- methylcyclopropyl)amino) but-2-enamide | 1H-NMR (400 MHz, CHLOROFORM- D) δ 9.98 (dd, J = 7.0, 1.3 Hz, 1H), 7.46- 7.42 (m, 3H), 7.17-7.10 (m, 2H), 7.06- 7.00 (m, 2H), 6.62 (s, 1H), 6.22 (dt, J = 1 5.3, 1.9 Hz, 1H), 4.86 (dd, J = 17.4, 10.9 Hz, 1H), 5.34 (dd, J = 17.3, 1.5 Hz, 1H), 4.68 (dd, J = 10.9, 1.4 Hz, 1H), 3.78 (s, 3H), 3.74 (s, 3H), 3.55 (dd, J = 5.0, 1.8 Hz, 2H), 2.64 (s, 3H), 1.29 (s, 3H), 0.62-0.59 (m, 2H), 0.42-0.40 (m, 2H) | 628.3 |
| 112-1 | 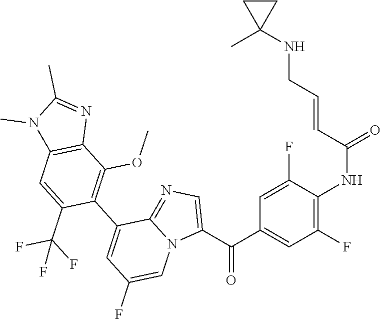 [SMILES: COc1c(-c2cc(F)cn3c(C(=O)c4cc(F)c(NC(=O)/C=C/CNC5(C)CC5)c(F)c4)cnc23)c(C(F)(F)F)cc2c1nc(C)n2C] | (E)-N-(2,6-difluoro-4-(6- fluoro-8-(4-methoxy-1,2- dimethyl-6- (trifluoromethyl)-1H- benzo[d]imidazol-5- yl)imidazo[1,2-a]pyridine- 3-carbonyl)phenyl)-4-((1- methylcyclopropyl)amino) but-2-enamide | 1H-NMR (400 MHz, CHLOROFORM- D) δ 9.73 (q, J = 2.3 Hz, 1H), 8.18 (s, 1H), 7.52-7.49 (m, 2H), 7.44-7.42 (m, 2H), 7.09 (td, J = 10.2, 5.1 Hz, 1H), 6.21 (d, J = 15.5 Hz, 1H), 4.16 (s, 3H), 3.78 (s, 3H), 3.55-3.41 (m, 2H), 2.65 (s, 3H), 1.28 (s, 3H), 0.63 (t, J = 4.9 Hz, 2H), 0.41 (dd, J = 6.0, 4.5 Hz, 2H) | 671.2 |
| 112-2 | 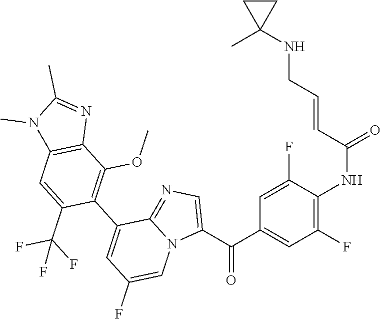 [SMILES: COc1c(-c2cc(F)cn3c(C(=O)c4cc(F)c(NC(=O)/C=C/CNC5(C)CC5)c(F)c4)cnc23)c(C(F)(F)F)cc2c1nc(C)n2C] | (E)-N-(2,6-difluoro-4-(6- fluoro-8-(4-methoxy-1,2- dimethyl-6- (trifluoromethyl)-1H- benzo[d]imidazol-5- yl)imidazo[1,2-a]pyridine- 3-carbonyl)phenyl)-4-((1- methylcyclopropyl)amino) but-2-enamide | 1H-NMR (400 MHz, CHLOROFORM- D) δ 9.73 (q, J = 2.3 Hz, 1H), 8.18 (s, 1H), 7.52-7.49 (m, 2H), 7.44-7.42 (m, 2H), 7.09 (td, J = 10.2, 5.1 Hz, 1H), 6.21 (d, J = 15.5 Hz, 1H), 4.16 (s, 3H), 3.78 (s, 3H), 3.55-3.41 (m, 2H), 2.65 (s, 3H), 1.28 (s, 3H), 0.63 (t, J = 4.9 Hz, 2H), 0.41 (dd, J = 6.0, 4.5 Hz, 2H) | 671.2 |
| 113 | 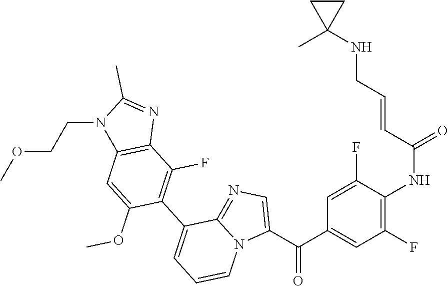 [SMILES: COCCn1c(C)nc2c(F)c(-c3cccn4c(C(=O)c5cc(F)c(NC(=O)/C=C/CNC6(C)CC6)c(F)c5)cnc34)c(OC)cc21] | (E)-N-(2,6-difluoro-4-(8- (4-fluoro-6-methoxy-1-(2- methoxyethyl)-2-methyl- 1H-benzo[d]imidazol-5- yl)indolizine-3- carbonyl)phenyl)-4-((1- methylcyclopropyl)amino) but-2-enamide | 1H-NMR (400 MHz, CHLOROFORM- D) δ 9.98 (d, J = 7.0 Hz, 1H), 7.41-7.38 (m, 3H), 7.31 (dd, J = 4.9, 1.1 Hz, 1H), 72.7-7.26 (m, 2H), 7.15-7.08 (m, 2H), 6.71 (s, 1H), 6.27-6.23 (m, 2H), 4.30 (t, J = 5.3 Hz, 2H), 3.77 (s, 3H), 3.74 (t, J = 5.3 Hz, 2H), 3.55-3.53 (m, 2H), 3.34 (d, J = 1.3 Hz, 3H), 2.64 (s, 3H), 1.29 (s, 3H), 0.60 (t, J = 5.0 Hz, 2H), 0.42-0.39 (m, 2H) | 645.9 |
| 114 | 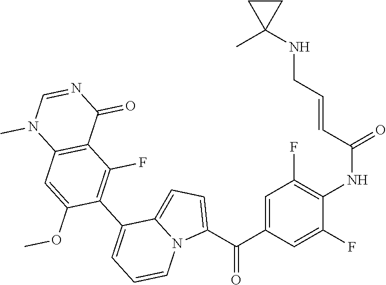 [SMILES: COc1cc2c(c(F)c1-c1cccn3c(C(=O)c4cc(F)c(NC(=O)/C=C/CNC5(C)CC5)c(F)c4)ccc13)c(=O)ncn2C] | (E)-N-(2,6-difluoro-4-(8- (5-fluoro-7-methoxy-1- methyl-4-oxo-1,4- dihydroquinazolin-6- yl)indolizine-3- carbonyl)phenyl)-4-((1- methylcyclopropyl)amino) but-2-enamide | 1H-NMR (400 MHz, DMSO-D6) δ 9.97 (s, 1H), 9.88 (d, J = 7.0 Hz, 1H), 8.45 (s, 1H), 7.58-7.52 (m, 2H), 7.44 (d, J = 4.8 Hz, 1H), 7.37 (dd, J = 7.0, 1.0 Hz, 1H), 7.28 (dd, J = 7.0, 7.0 Hz, 1H), 6.98 (s, 1H), 6.88 (td, J = 10.2, 5.2 Hz, 1H), 6.30 (dt, J = 15.3, 1.8 Hz, 1H), 6.23 (d, J = 4.8 Hz, 1H), 3.90 (s, 3H), 3.80 (s, 3H), 3.40 (dd, J = 5.1, 1.4 Hz, 2H), 1.21 (s, 3H), 0.49 (dd, J = 5.8, 4.0 Hz, 2H), 0.31 (dd, J = 6.0, 4.0 Hz, 2H) | 616.2 |
| 115 | 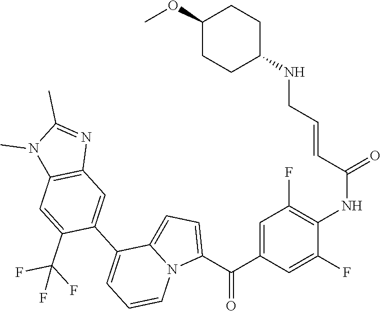 [SMILES: CO[C@H]1CC[C@H](NC/C=C/C(=O)Nc2c(F)cc(C(=O)c3ccc4c(-c5cc6nc(C)n(C)c6cc5C(F)(F)F)cccn34)cc2F)CC1] | (E)-N-(4-(1-(1,2- dimethyl-6- (trifluoromethyl)-1H- benzo[d]imidazol-5- yl)pyrrolo[1,2-a]pyrazine- 6-carbonyl)-2,6- difluorophenyl)-4- (((1r,4r)-4- methoxycyclohexyl)amino) but-2-enamide | 1H-NMR (400 MHz, CHLOROFORM- D) δ 9.61 (d, J = 4.8 Hz, 1H), 8.13 (d, J = 5.0 Hz, 1H), 7.81 (d, J = 6.0 Hz, 2H), 7.50 (t, J = 7.9 Hz, 2H), 7.38 (d, J = 5.0 Hz, 1H), 7.13 (td, J = 10.3, 5.1 Hz, 1H), 6.47 (d, J = 4.8 Hz, 1H), 6.25 (d, J = 15.8 Hz, 1H), 3.88 (s, 3H), 3.52 (d, J = 3.8 Hz, 2H), 3.35 (s, 3H), 3.18-3.13 (m, 1H), 2.71 (s, 3H), 2.60-2.55 (m, 1H), 2.10- 1.98 (m, 4H), 1.28-1.21 (m, 4H) | 681.2 |
| 116 | 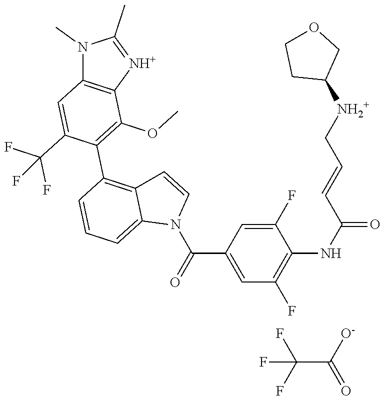 [SMILES: COc1c(-c2cccc3c2ccn3C(=O)c2cc(F)c(NC(=O)/C=C/C[NH2+][C@H]3CCOC3)c(F)c2)c(C(F)(F)F)cc2c1[nH+]c(C)n2C ; O=C([O-])C(F)(F)F] | mono(5-(1-(3,5-difluoro- 4-((E)-4-(((S)- tetrahydrofuran-3- yl)ammonio)but-2- enamido)benzoyl)-1H- indol-4-yl)-4-methoxy- 1,2-dimethyl-6- (trifluoromethyl)-1H- benzo[d]imidazol-3-ium) mono(2,2,2- trifluoroacetate) | ¹H NMR (499 MHz, MeOD) δ 8.48 (d, J = 8.4 Hz, 1H), 7.99 (s, 1H), 7.57 (d, J = 7.6 Hz, 2H), 7.4 9(t, J = 7.9 Hz, 1H), 7.41 (d, J = 3.8 Hz, 1H), 7.28 (d, J = 7.2 Hz, 1H), 7.01-6.87 (m, 1H), 6.63 (d, J = 15.4 Hz, 1H), 6.22 (d, J = 3.7 Hz, 1H), 4.16-3.92 (m, 9H), 3.89-3.81 (m, 1H), 3.77 (q, J = 8.3 Hz, 1H), 3.71 (s, 3H), 2.84 (s, 3H), 2.44 (m, 1H), 2.07 (m, 1H). [M + H]⁺ m/z 668. | 668 |
| 117 | 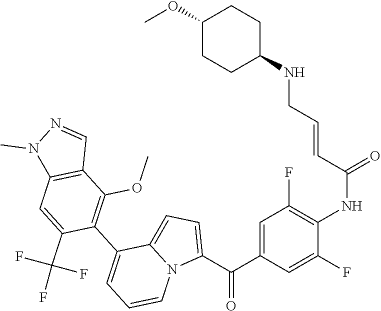 [SMILES: COc1c(-c2cccn3c(C(=O)c4cc(F)c(NC(=O)/C=C/CN[C@H]5CC[C@H](OC)CC5)c(F)c4)ccc23)c(C(F)(F)F)cc2c1cnn2C] | (E)-N-(2,6-difluoro-4-(8- (4-methoxy-1-methyl-6- (trifluoromethyl)-1H- indazol-5-yl)indolizine-3- carbonyl)phenyl)-4- (((1r,4r)-4- methoxycyclohexyl)amino) but-2-enamide | ¹H NMR (400 MHz, MeOD) δ 9.94 (d, J = 6.85 Hz, 1H), 8.43 (s, 1H), 7.78 (s, 1H), 7.49 (d, J = 8.07 Hz, 2H), 7.37 (d, J = 4.89 Hz, 1H), 7.21-7.25 (m, 1H), 7.15- 7.20 (m, 1H), 7.00 (td, J = 5.93, 15.53 Hz, 1H), 6.37 (d, J = 15.41 Hz, 1H), 6.09 (d, J = 4.65 Hz, 1H), 4.18 (s, 3H), 4.06 (s, 3H), 3.55 (d, J = 5.38 Hz, 2H), 3.34-3.36 (m, 3H), 3.13-3.26 (m, 1H), 2.56-2.69 (m, 1H), 2.02-2.13 (m, 4H), 1.20-1.26 (m, 4H). | 696.2 |
| 118 | 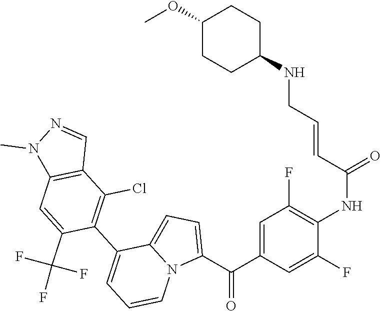 [SMILES: CO[C@H]1CC[C@H](NC/C=C/C(=O)Nc2c(F)cc(C(=O)c3ccc4c(-c5c(C(F)(F)F)cc6c(cnn6C)c5Cl)cccn34)cc2F)CC1] | (E)-N-(4-(8-(4-chloro-1- methyl-6- (trifluoromethyl)-1H- indazol-5-yl)indolizine-3- carbonyl)-2,6- diflurophenyl)-4- (((1r,4r)-4- methoxycyclohexyl)amino) but-2-enamide | 1H NMR (400 MHz, MeOD) δ 10.00 (d, J = 7.09 Hz, 1H), 8.23 (d, J = 6.11 Hz, 2H), 7.49 (d, J = 7.83 Hz, 2H), 7.42 (d, J = 4.89 Hz, 1H), 7.26-7.30 (m, 1H), 7.19-7.25 (m, 1H), 7.01 (td, J = 5.96, 15.47 Hz, 1H), 6.34 (d, J = 15.41 Hz, 1H), 6.06 (d, J = 4.65 Hz, 1H), 4.24 (s, 3H), 3.49 (d, J = 5.14 Hz, 2H), 3.33-3.35 (m, 3H), 3.13-3.24 (m, 1H), 2.54 (dd, J = 2.81, 6.97 Hz, 1H), 2.00-2.11 (m, 4H), 1.17-1.25 (m, 4H). | 700.2 |
| 119 | 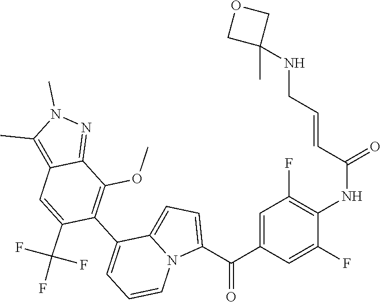 [SMILES: COc1c(-c2cccn3c(C(=O)c4cc(F)c(NC(=O)/C=C/CNC5(C)COC5)c(F)c4)ccc23)c(C(F)(F)F)cc2c(C)n(C)nc12] | (E)-N-(2,6-difluoro-4-(8- (7-methoxy-2,3-dimethyl- 5-(trifluoromethyl)-2H- indazol-6-yl)indolizine-3- carbonyl)phenyl)-4-((3- methyloxetan-3- yl)amino)but-2-enamide | ¹H NMR (500 MHz, CD3OD) δ 9.97 (d, J = 7.0 Hz, 1H), 7.97 (s, 1H), 7.52 (d, J = 7.9 Hz, 2H), 7.40 (d, J = 4.9 Hz, 1H), 7.33-7.18 (m, 2H), 7.11-6.90 (m, 1H), 6.66 (br d, J = 15.4 Hz, 1H), 6.17 (d, J = 4.7 Hz, 1H), 4.81 (br d, J = 7.6 Hz, 2H), 4.60 (d, J = 7.8 Hz, 2H), 4.25-4.15 (m, 3H), 4.03-3.95 (m, 5H), 2.76 (s, 3H), 1.85- 1.68 (m, 3H). | 668.2 |
| 120 | 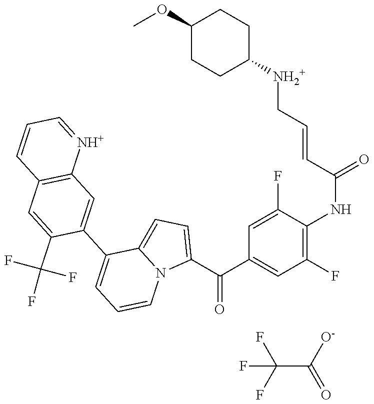 [SMILES: CO[C@H]1CC[C@H]([NH2+]C/C=C/C(=O)Nc2c(F)cc(C(=O)c3ccc4c(-c5cc6[nH+]cccc6cc5C(F)(F)F)cccn34)cc2F)CC1 ; O=C([O-])C(F)(F)F] | mono(7-(3-(3,5-difluoro- 4-((E)-4-(((1r,4r)-4- methoxycyclohexyl)ammo- nio)but-2- enamido)benzoyl)indolizin- 8-yl)-6- (trifluoromethyl)quinolin- 1-ium) mono(2,2,2- trifluoroacetate | 1H NMR (400 MHz, MeOD) δ 10.02 (d, J = 7.0 Hz, 1H), 9.11 (d, J = 3.0 Hz, 1H), 86.1-8.79 (m, 2H), 8.17 (s, 1H), 7.79 (dd, J = 8.4, 4.4 Hz, 1H), 7.55 (d, J = 8.0 Hz, 2H), 7.46 (d, J = 4.9 Hz, 1H), 7.39 (d, J = 6.9 Hz, 1H), 7.23-7.33 (m, 1H), 6.86- 7.03 (m, 1H), 6.61 (d, J = 15.4 Hz, 1H), 6.22 (d, J = 4.8 Hz, 1H), 3.88-4.00 (m, 1H), 3.96 (d, J = 6.6 Hz, 1H), 3.38 (s, 3H), 3.12-3.29 (m, 2H), 2.22 (br d, J = 9.1 Hz, 2H), 2.12-2.32 (m, 1H), 2.12-2.34 (m, 1H), 1.42-1.54 (m, 1H), 1.39-1.57 (m, 1H), 1.26-1.37 (m, 2H). | 663.3 |
| 121 | 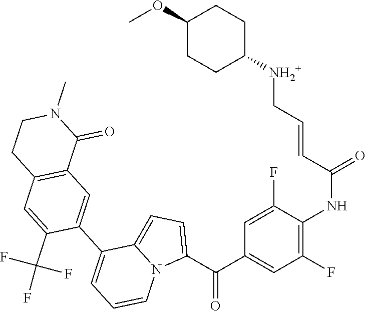 [SMILES: CO[C@H]1CC[C@H]([NH2+]C/C=C/C(=O)Nc2c(F)cc(C(=O)c3ccc4c(-c5cc6c(cc5C(F)(F)F)CCN(C)C6=O)cccn34)cc2F)CC1] | (1r,4r)-N-((E)-4-((2,6- difluoro-4-(8-(2-methyl-1- oxo-6-(trifluoromethyl)- 1,2,3,4- tetrahydroisoquinolin-7- yl)indolizine-3- carbonyl)phenyl)amino)- 4-oxobut-2-en-1-yl)-4- methoxycyclohexan-1- aminiun 2,2,2- trifluoroacetate | ¹H NMR (400 MHz, MeOD) δ 9.96 (d, J = 6.9 Hz, 1H), 8.00 (s, 1H), 7.85 (s, 1H), 7.52 (d, J = 7.8 Hz, 2H), 7.43 (d, J = 4.9 Hz, 1H), 7.23-7.26 (m, 1H), 7.18-7.22 (m, 1H), 6.93 (dt, J = 15.3, 6.8 Hz, 1H), 6.59 (d, J = 15.4 Hz, 1H), 6.17 (d, J = 4.9 Hz, 1H), 3.94 (d, J = 6.9 Hz, 2H), 3.74 (t, J = 6.7 Hz, 2H), 3.36 (s, 3H), 3.20-3.25 (m, 3H), 3.13-3.19 (m, 4H), 2.20 (br d, J = 10.4 Hz, 4H), 1.46 (q, J = 11.6 Hz, 2H), 1.27-1.34 (m, 2H). | 695.2 |
| 122 | 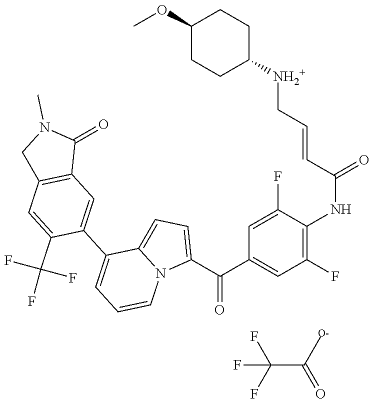 [SMILES: CO[C@H]1CC[C@H]([NH2+]C/C=C/C(=O)Nc2c(F)cc(C(=O)c3ccc4c(-c5cc6c(cc5C(F)(F)F)CN(C)C6=O)cccn34)cc2F)CC1 ; O=C([O-])C(F)(F)F] | (1r,4r)-N-((E)-4-((2,6- difluoro-4-(8-(2-methyl-3- oxo-6- (trifluoromethyl)isoindolin- 5-yl)indolizine-3- carbonyl)phenyl)amino)- 4-oxobut-2-en-1-yl)-4- methoxycyclohexan-1- aminium 2,2,2- trifluoroacetate | ¹H NMR (400 MHz, MeOD) δ 9.99 (d, J = 7.0 Hz, 1H), 8.18 (s, 1H), 7.85 (s, 1H), 7.54 (d, J = 7.9 Hz, 2H), 7.45 (d, J = 4.9 Hz, 1H), 7.30 (d, J = 7.0 Hz, 1H), 7.21-7.27 (m, 1H), 6.94 (dt, J = 15.4, 6.8 Hz, 1H), 6.61 (d, J = 15.4 Hz, 1H), 6.17 (d, J = 4.9 Hz, 1H), 4.72 (s, 2H), 3.96 (d, J = 7.3 Hz, 2H), 3.38 (s, 3H), 3.28 (s, 3H), 3.21-3.26 (m, 1H), 3.15-3.21 (m, 1H), 2.22 (br d, J = 12.1 Hz, 4H), 1.43-1.53 (m, 2H), 1.29- 1.39 (m, 2H). | 681.2 |
| 123 | 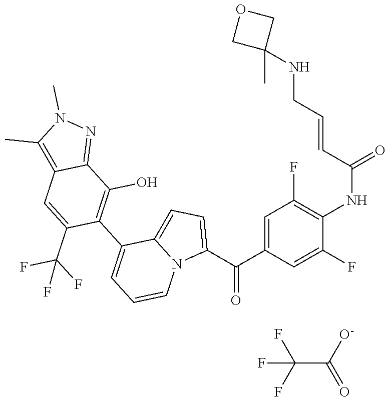 [SMILES: Cc1c2cc(C(F)(F)F)c(-c3cccn4c(C(=O)c5cc(F)c(NC(=O)/C=C/CNC6(C)COC6)c(F)c5)ccc34)c(O)c2nn1C ; O=C([O-])C(F)(F)F] | (E)-N-(4-((2,6-difluoro-4- (8-(7-hydroxy-2,3- dimethyl-5- (trifluoromethyl)-2H- indazol-6-yl)indolizine-3- carbonyl)phenyl)amino)- 4-oxobut-2-en-1-yl)-3- methyloxetan-3-aminium 2,2,2-trifluoroacetate | 1H NMR (400 MHz, MeOD) δ 9.96 (br d, J = 6.8 Hz, 1H), 7.73 (s, 1H), 7.50 (br d, J = 8.1 Hz, 2H), 7.39-7.35 (m, 1H), 7.31- 7.26 (m, 1H), 7.24-7.14 (m, 1H), 7.04- 6.89 (m, 1H), 6.65 (br d, J = 15.2 Hz, 1H), 6.15 (d, J = 4.6 Hz, 1H), 4.80 (br d, J = 7.8 Hz, 2H), 4.58 (d, J = 7.8 Hz, 2H), 4.15 (s, 3H), 3.97 (br d, J = 6.8 Hz, 2H), 2.70 (d, J = 1.7 Hz, 3H), 1.74 (s, 3H). | 654.1 |
| 124 | 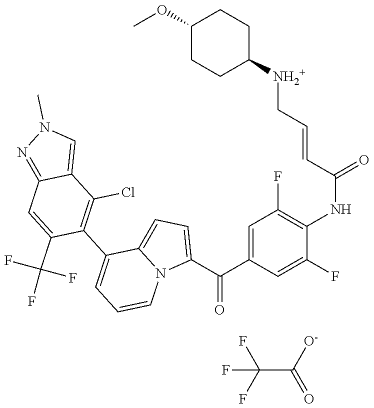 [SMILES: CO[C@H]1CC[C@H]([NH2+]C/C=C/C(=O)Nc2c(F)cc(C(=O)c3ccc4c(-c5c(C(F)(F)F)cc6nn(C)cc6c5Cl)cccn34)cc2F)CC1 ; O=C([O-])C(F)(F)F] | (1r,4r)-N-((E)-4-((4-(8-(4- chloro-2-methyl-6- (trifluoromethyl)-2H- indazol-5-yl)indolizine-3- carbonyl)-2,6- difluorophenyl)amino)-4- oxobut-2-en-1-yl)-4- methoxycyclohexan-1- aminium 2,2,2- trifluoroacettate | 1H NMR (400 MHz, MeOD) δ 10.01 (d, J = 6.9 Hz, 1H), 8.55 (s, 1H), 8.20 (s, 1H), 7.53 (d, J = 7.9 Hz, 2H), 7.43 (d, J = 4.9 Hz, 1H), 7.36-7.29 (m, 1H), 7.28-7.21 (m, 1H), 6.94 (td, J = 6.8, 15.3 Hz, 1H), 6.61 (d, J = 15.4 Hz, 1H), 6.12 (d, J = 4.8 Hz, 1H), 4.36 (s, 3H), 3.96 (br d, J = 6.7 Hz, 2H), 3.38 (s, 3H), 3.29-3.07 (m, 2H), 2.22 (br d, J = 9.2 Hz, 4H), 1.62-1.41 (m, 2H), 1.40-1.20 (m, 2H). | 701.1 |
| 125 | 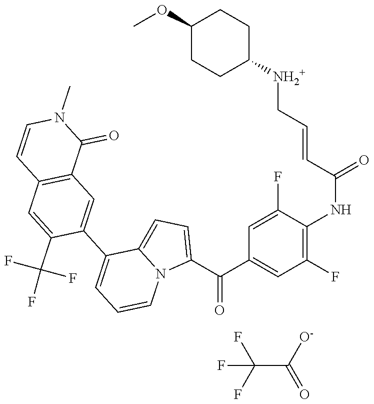 [SMILES: CO[C@H]1CC[C@H]([NH2+]C/C=C/C(=O)Nc2c(F)cc(C(=O)c3ccc4c(-c5cc6c(=O)n(C)ccc6cc5C(F)(F)F)cccn34)cc2F)CC1 ; O=C([O-])C(F)(F)F] | (1r,4r)-N-((E)-4-((2,6- difluoro-4-(8-(2-methyl-1- oxo-6-(trifluoromethyl)- 1,2-dihydroisoquinolin-7- yl)indolizine-3- carobnyl)phenyl)amino)- 4-oxobut-2-en-1-yl)-4- methoxycyclohexan-1- aminium 2,2,2- trifluoroacetate | ¹H NMR (400 MHz, MeOD) δ 9.98 (d, J = 7.0 Hz, 1H), 8.38 (s, 1H), 8.25 (s, 1H), 7.58 (d, J = 7.3 Hz, 1H), 7.52 (d, J = 7.9 Hz, 2H), 7.43 (d, J = 4.9 Hz, 1H), 7.31 (d, J = 7.0 Hz, 1H), 7.17-7.25 (m, 1H), 6.88- 6.96 (m, 2H), 6.59 (d, J = 15.4 Hz, 1H), 6.18 (d, J = 4.9 Hz, 1H), 3.94 (d, J = 6.7 Hz, 2H), 3.66 (s, 2H), 3.64-3.67 (m, 1H), 3.36 (s, 3H), 3.21-3.26 (m, 1H), 3.13- 3.19 (m, 1H), 2.20 (br d, J = 10.1 Hz, 4H), 1.40-1.51 (m, 2H), 1.31 (br d, J = 12.8 Hz, 2H). | 693.2 |
| 126 | 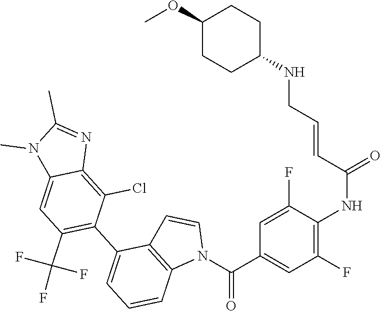 [SMILES: CO[C@H]1CC[C@H](NC/C=C/C(=O)Nc2c(F)cc(C(=O)n3ccc4c(-c5c(C(F)(F)F)cc6c(nc(C)n6C)c5Cl)cccc43)cc2F)CC1] | (E)-N-(4-(4-(4-chloro-1,2- dimethyl-6- (trifluoromethyl)-1H- benzo[d]imidazol-5-yl)- 1H-indole-1-carbonyl)- 2,6-difluorophenyl)-4- (((1r,4r)-4- methoxycyclohexyl)amino) but-2-enamide | ¹H NMR (400 MHz, CDCl₃) δ 8.50 (d, J = 8.34 Hz, 1H), 7.72 (s, 1H), 7.50 (t, J = 7.87 Hz, 1H), 7.41 (d, J = 7.39 Hz, 2H), 7.22-7.26 (m, 2H), 7.11 (td, J = 5.10, 15.32 Hz, 1H), 6.31 (d, J = 15.38 Hz, 1H), 6.14 (d, J = 3.70 Hz, 1H), 3.88 (s, 3H), 3.55 (d, J = 4.41 Hz, 2H), 3.35 (s, 3H), 3.08-3.19 (m, 1H), 2.74 (s, 3H), 2.57- 2.67 (m, 1H), 2.10 (d, J = 9.30 Hz, 2H), 2.01-2.04 (m, 2H), 1.19-1.30 (m, 4H). | 714.2 |
| 127 | 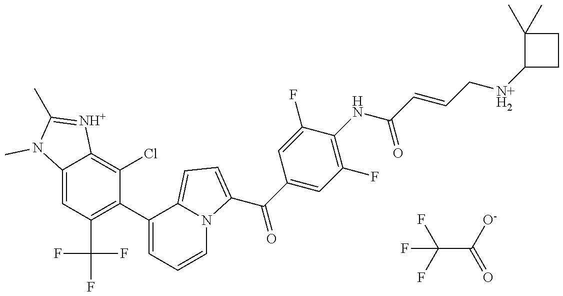 | mono((E)-4-chloro-5-(3- (4-(4-((2,2- dimethylcyclobutyl)ammo- nio)but-2-enamido)-3,5- difluorobenzoyl)indolizin- 8-yl)-1,2-dimethyl-6- (trifluoromethyl)-1H- benzo[d]imidazol-3-ium) mono(2,2,2- trifluoroacetate) | 1H NMR (499 MHz, DMSO-d6) δ 10.35 (s, 1H), 9.92 (t, J = 3.8 Hz, 1H), 8.95 (br. s, 1H), 8.71 (br. s, 1H), 8.20 (s, 1H), 7.60 (d, J = 7.9 Hz, 2H), 7.42 (d, J = 4.7 Hz, 1H), 7.31 (d, J = 4.0 Hz, 2H), 6.91 (dt, J = 15.5, 4.9 Hz, 1H), 6.53 (d, J = 15.3 Hz, 1H), 6.01 (d, J = 4.6 Hz, 1H), 3.92 (s, 3H), 3.77 (br. s, 2H), 2.65 (s, 3H), 2.17- 2.00 (m, 1H), 1.98-1.59 (m, 1H), 1.62- 1.59 (m, 1H), 1.25 (s, 3H), 1.17 (s, 3H). | 684 |
| 128 | 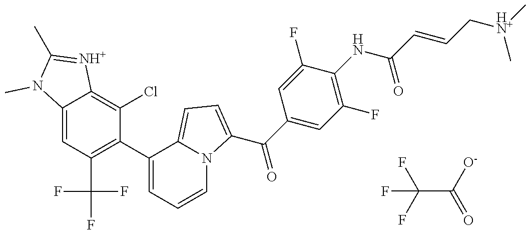 | mono((E)-4-chloro-5-(3- (4-(4- (dimethylammonio)but-2- enamido)-3,5- difluorobenzoyl)indolizin- 8-yl)-1,2-dimethyl-6- (trifluoromethyl)-1H- benzo[d]imidazol-3-ium) mono(2,2,2- trifluoroacetate) | | 630 |
| 129 | 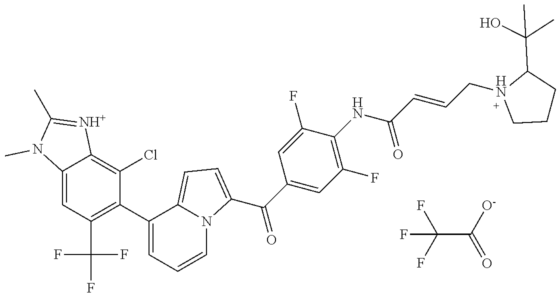 | mono((E)-4-chloro-5-(3- (3,5-difluoro-4-(4-(2-(2- hydroxypropan-2- yl)pyrrolidin-1-ium-1- yl)but-2- enamido)benzoyl)indolizin- 8-yl)-1,2-dimethyl-6- (trifluoromethyl)-1H- benzo[d]imidazol-3-ium) mono(2,2,2- trifluoroacetate) | | 714 |
| 130 | 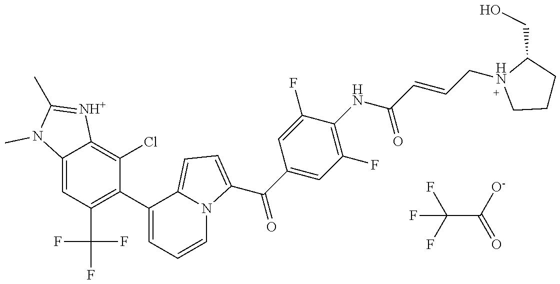 | mono(4-chloro-5-(3-(3,5- difluoro-4-((E)-4-((2S)-2- (hydroxymethyl)pyrrolidin- 1-ium-1-yl)but-2- enamido)benzoyl)indolizin- 8-yl)-1,2-dimethyl-6- (trifluoromethyl)-1H- benzo[d]imidazol-3-ium) mono(2,2,2- triflurooacetate) | | 686 |
| 131a | 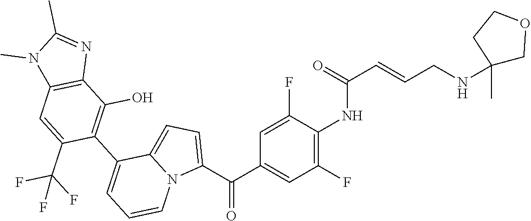 [SMILES: Cc1nc2c(O)c(-c3cccn4c(C(=O)c5cc(F)c(NC(=O)/C=C/CNC6(C)CCOC6)c(F)c5)ccc34)c(C(F)(F)F)cc2n1C] | (E)-N-(4-(8-(4-chloro-1,2- dimethyl-6- (trifluoromethyl)-1H- benzo[d]imidazol-5- yl)indolizine-3-carbonyl)- 2,6-difluorophenyl)-4-((3- methyltetrahydrofuran-3- yl)amino)but-2-enamide | 1H NMR (499 MHz, DMSO-d6) δ 10.00 (s, 1H), 9.92 (t, J = 3.8 Hz, 1H), 8.20 (s, 1H), 7.56 (d, J = 7.9 Hz, 2H), 7.43 (d, J = 4.7 Hz, 1H), 7.31 (d, J = 4.0 Hz, 2H), 6.91 (dt, J = 15.5, 4.9 Hz, 1H), 6.38 (d, J = 15.3 Hz, 1H), 6.00 (d, J = 4.6 Hz, 1H), 3.92 (s, 3H), 3.85 (q, J = 7.8 Hz, 1H), 3.74 (q, J = 8.0 Hz, 1H), 3.54 (d, J = 8.2 Hz, 2H), 3.36 (d, J = 7.6 Hz, 3H), 2.66 (s, 3H), 1.89 (dt, J = 13.7, 7.0 Hz, 1H), 1.73- 1.63 (m, 1H), 1.21 (s, 3H). | 686.1 |
| 131b | 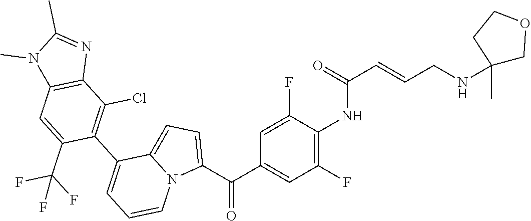 [SMILES: Cc1nc2c(Cl)c(-c3cccn4c(C(=O)c5cc(F)c(NC(=O)/C=C/CNC6(C)CCOC6)c(F)c5)ccc34)c(C(F)(F)F)cc2n1C] | (E)-N-(4-(8-(4-chloro-1,2- dimethyl-6- (trifluoromethyl)-1H- benzo[d]imidazol-5- yl)indolizine-3-carobnyl)- 2,6-difluorophenyl)-4-((3- methyltetrahydrofuran-3- yl)amino)but-2-enamide | 1H NMR (499 MHz, DMSO-d6) δ 10.02 (s, 1H), 9.92 (s, 1H), 8.21 (s, 1H), 7.57 (d, J = 7.4 Hz, 2H), 7.43 (s, 1H), 7.31 (s, 2H), 6.91 (d, J = 15.9 Hz, 1H), 6.38 (d, J = 15.1 Hz, 1H), 6.00 (s, 1H), 3.92 (s, 3H), 3.84 (s, 1H), 3.75 (s, 1H), 3.54 (d, J = 7.7 Hz, 2H), 3.38 (br. s, 3H), 2.66 (s, 3H), 1.89 (s, 1H), 1.69 (s, 1H), 1.21 (s, 3H). | 686 |
| 132 | 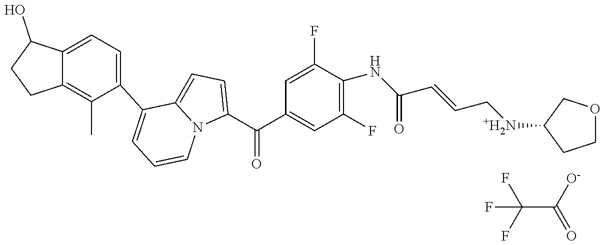 [SMILES: Cc1c(-c2cccn3c(C(=O)c4cc(F)c(NC(=O)/C=C/C[NH2+][C@H]5CCOC5)c(F)c4)ccc23)ccc2c1CCC2O ; O=C([O-])C(F)(F)F] | (3S)-N-((E)-4-((2,6- difluoro-4-(8-(1-hydroxy- 4-methyl-2,3-dihydro-1H- inden-5-yl)indolizine-3- carbonyl)phenyl)amino)- 4-oxobut-2-en-1- yl)tetrahydrofuran-3- aminium 2,2,2- trifluoroacetate | 1H NMR (499 MHz, DMSO) δ 10.35 (s, 1H), 9.87 (d, J = 7.3 Hz, 1H), 9.09 (s, 2H), 7.59 (d, J = 7.9 Hz, 2H), 7.44 (d, J = 4.3 Hz, 1H), 7.26 (dd, J = 15.8, 7.5 Hz, 3H), 6.82 (dt, J = 13.5, 6.6 Hz, 1H), 6.54 (d, J = 15.4 Hz, 1H), 6.22 (d, J = 4.7 Hz, 1H), 5.09 (s, 1H), 3.91 (d, J = 6.6 Hz, 5H), 3.78 (dd, J = 11.0, 6.4 Hz, 2H), 3.73- 3.62 (m, 2H), 2.96 (d, J = 8.9 Hz, 1H), 2.77 (dt, J = 15.9, 7.9 Hz, 1H), 2.38 (d, J = 7.3 Hz, 1H), 2.26 (dq, J = 14.2, 7.9 Hz, 1H), 2.10 (s, 3H), 2.02 (s, 1H), 1.84 (s, 1H). | 572 |

In some instances, the compounds presented in the Examples and in the table above were isolated and are represented as salts. e.g., trifluoroacetate salts such as Ex. 122. One of ordinary skill in the art will appreciate that, depending on the isolation conditions, the un-ionized form of the compound could be isolated, or a salt form having a counterion other than trifluoroacetate could be isolated. Such un-ionized forms of the compound and alternative salt forms of the compound are within the scope of the present disclosure. Such un-ionized forms of the compound include, without limitation:

| | |
|---|---|
| 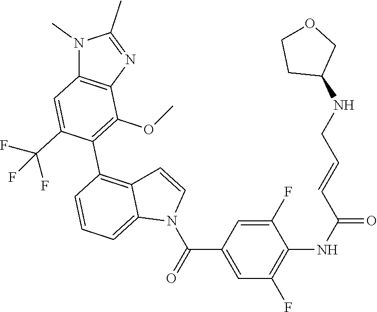 [SMILES: COc1c(-c2cccc3c2ccn3C(=O)c2cc(F)c(NC(=O)/C=C/CN[C@H]3CCOC3)c(F)c2)c(C(F)(F)F)cc2c1nc(C)n2C] | (E)-N-(2,6-difluoro-4-(4-(4-methoxy- 1,2-dimethyl-6-(trifluoromethyl)-1H- benzo[d]imidazol-5-yl)-1H-indole-1- carbonyl)phenyl)-4-(((S)- tetrahydrofuran-3-yl)amino)but-2- enamide |
| 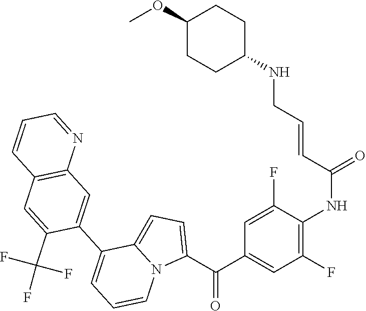 [SMILES: CO[C@H]1CC[C@H](NC/C=C/C(=O)Nc2c(F)cc(C(=O)c3ccc4c(-c5cc6ncccc6cc5C(F)(F)F)cccn34)cc2F)CC1] | (E)-N-(2,6-difluoro-4-(8-(6- (trifluoromethyl)quinolin-7-yl)indolizine- 3-carbonyl)phenyl)-4-(((1r,4r)-4- methoxycyclohexyl)amino)but-2- enamide |
| 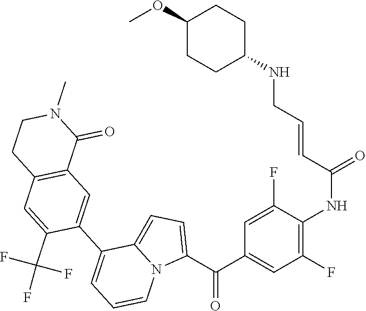 [SMILES: CO[C@H]1CC[C@H](NC/C=C/C(=O)Nc2c(F)cc(C(=O)c3ccc4c(-c5cc6c(cc5C(F)(F)F)CCN(C)C6=O)cccn34)cc2F)CC1] | (E)-N-(2,6-difluoro-4-(8-(2-methyl-1- oxo-6-(trifluoromethyl)-1,2,3,4- tetrahydroisoquinolin-7-yl)indolizine-3- carbonyl)phenyl)-4-(((1r,4r)-4- methoxycyclohexyl)amino)but-2- enamide |
| 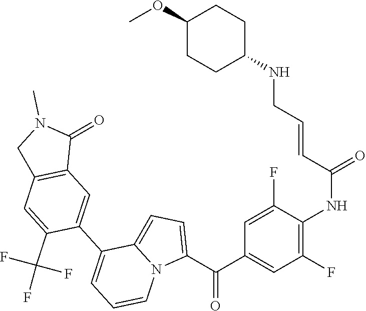 [SMILES: CO[C@H]1CC[C@H](NC/C=C/C(=O)Nc2c(F)cc(C(=O)c3ccc4c(-c5cc6c(cc5C(F)(F)F)CN(C)C6=O)cccn34)cc2F)CC1] | (E)-N-(2,6-difluoro-4-(8-(2-methyl-3- oxo-6-(trifluoromethyl)isoindolin-5- yl)indolizine-3-carbonyl)phenyl)-4- (((1r,4r)-4- methoxycyclohexyl)amino)but-2- enamide |
| 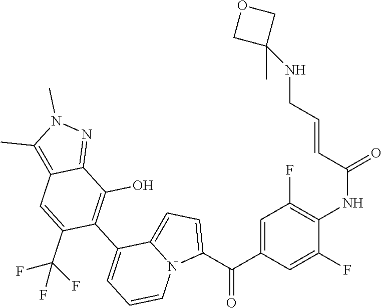 [SMILES: Cc1c2cc(C(F)(F)F)c(-c3cccn4c(C(=O)c5cc(F)c(NC(=O)/C=C/CNC6(C)COC6)c(F)c5)ccc34)c(O)c2nn1C] | (E)-N-(2,6-difluoro-4-(8-(7-hydroxy-2,3- dimethyl-5-(trifluoromethyl)-2H-indazol- 6-yl)indolizine-3-carbonyl)phenyl)-4-((3- methyloxetan-3-yl)amino)but-2-enamide |
| 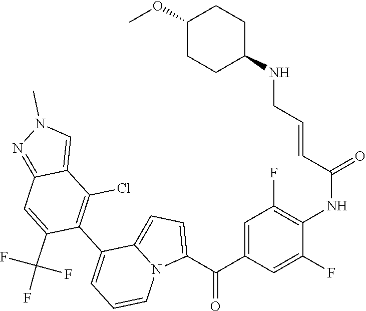 [SMILES: CO[C@H]1CC[C@H](NC/C=C/C(=O)Nc2c(F)cc(C(=O)c3ccc4c(-c5c(C(F)(F)F)cc6nn(C)cc6c5Cl)cccn34)cc2F)CC1] | (E)-N-(4-(8-(4-chloro-2-methyl-6- (trifluoromethyl)-2H-indazol-5- yl)indolizine-3-carbonyl)-2,6- difluorophenyl)-4-(((1r,4r)-4- methoxycyclohexyl)amino)but-2- enamide |
| 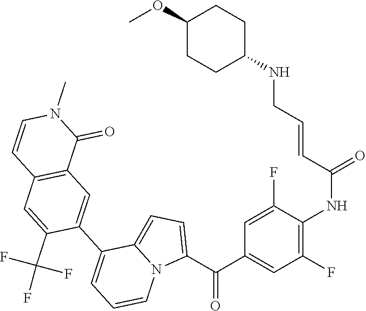 [SMILES: CO[C@H]1CC[C@H](NC/C=C/C(=O)Nc2c(F)cc(C(=O)c3ccc4c(-c5cc6c(=O)n(C)ccc6cc5C(F)(F)F)cccn34)cc2F)CC1] | (E)-N-(2,6-difluoro-4-(8-(2-methyl-1- oxo-6-(trifluoromethyl)-1,2- dihydroisoquinolin-7-yl)indolizine-3- carbonyl)phenyl)-4-(((1,4r)-4- methoxycyclohexyl)amino)but-2- enamide |
| 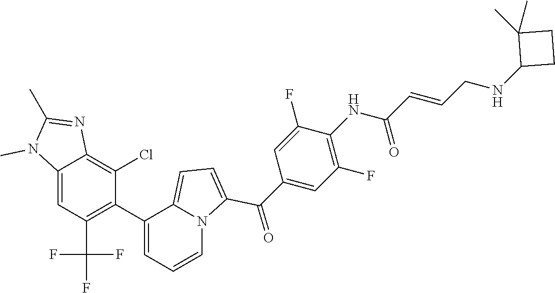 [SMILES: Cc1nc2c(Cl)c(-c3cccn4c(C(=O)c5cc(F)c(NC(=O)/C=C/CNC6CCC6(C)C)c(F)c5)ccc34)c(C(F)(F)F)cc2n1C] | (E)-N-(4-(8-(4-chloro-1,2-dimethyl-6- (trifluoromethyl)-1H-benzo[d]imidazol- 5-yl)indolizine-3-carbonyl)-2,6- difluorophenyl)-4-((2,2- dimethylcyclobutyl)amino)but-2-enamide |
| 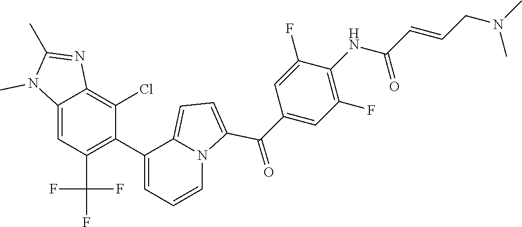 | (E)-N-(4-(8-(4-chloro-1,2-dimethyl-6- (trifluoromethyl)-1H-benzo[d]imidazol-5- yl)indolizine-3-carbonyl)-2,6- difluorophenyl)-4-(dimethylamino)but-2- enamide |
| 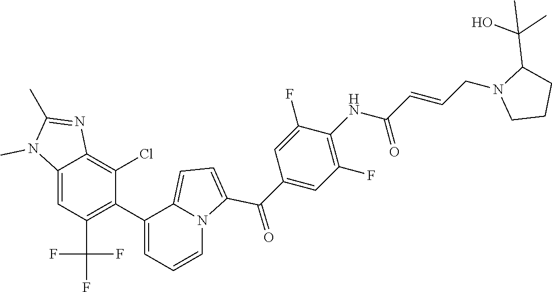 [SMILES: Cc1nc2c(Cl)c(-c3cccn4c(C(=O)c5cc(F)c(NC(=O)/C=C/CN6CCCC6C(C)(C)O)c(F)c5)ccc34)c(C(F)(F)F)cc2n1C] | (E)-N-(4-(9,10-dimethyl-7-(trifluoromethyl)- 13,14-dihydro-9H- imidazo[4″,5″:5′,6′]benzo[1′,2′:2,3]oxocino[4, 5,6-hi]indolizine-2-carbonyl)-2,6- difluorophenyl)-3-(pyrrolidin-2- yl)acrylamide |
| 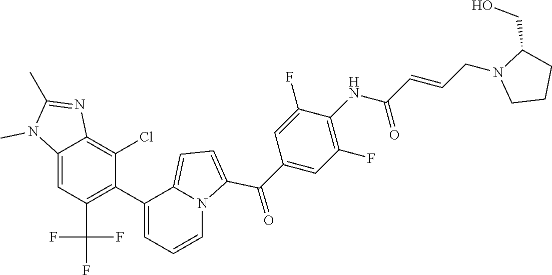 [SMILES: Cc1nc2c(Cl)c(-c3cccn4c(C(=O)c5cc(F)c(NC(=O)/C=C/CN6CCC[C@H]6CO)c(F)c5)ccc34)c(C(F)(F)F)cc2n1C] | (S,E)-N-(4-(8-(4-chloro-1,2-dimethyl-6- (trifluoromethyl)-1H-benzo[d]imidazol-5- yl)indolizine-3-carbonyl)-2,6- difluorophenyl)-4-(2- (hydroxymethyl)pyrrolidin-1-yl)but-2- enamide |
| 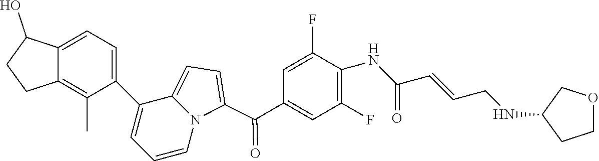 [SMILES: Cc1c(-c2cccn3c(C(=O)c4cc(F)c(NC(=O)/C=C/CN[C@H]5CCOC5)c(F)c4)ccc23)ccc2c1CCC2O] | (E)-N-(2,6-difluoro-4-(8-(1-hydroxy-4- methyl-2,3-dihydro-1H-inden-5- yl)indolizine-3-carbonyl)phenyl)-4-(((R)- tetrahydrofuran-3-yl)amino)but-2- enamide ((132)) |

The compounds disclosed herein may contain one or more asymmetric centers and can thus occur as racemates and racemic mixtures, single enantiomers, diastereoisomeric mixtures and individual diastereoisomers. Centers of asymmetry that are present in the compounds disclosed herein can all independently of one another have S configuration or R configuration. The compounds disclosed herein include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds disclosed herein. Where a structural formula or chemical name specifies a particular configuration at a stereocenter, the enantiomer or stereoisomer of the compound resulting from that specified stereocenter is intended. Where a structural formula of the compounds disclosed herein indicates a straight line at a chiral center, the structural formula includes both the S and R stereoisomers associated with the chiral center and mixtures thereof.

The compounds disclosed herein may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds disclosed herein may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds disclosed herein which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds disclosed herein.

Some of the compounds described herein may exist as atropisomers when the rotational energy barrier around a single bond is sufficiently high to prevent free rotation at a given temperature, thus allowing isolation of individual conformers with distinct properties. A typical example of stable atropisomers are exemplified by Example 12-1 and 12-2 (described below), which can be resolved by chiral chromatography separation. The individual atropisomers as well as mixtures thereof are encompassed with compounds of the present disclosure. When resolved, individual atropisomers can be designated by established conventions such as those specified by the International Union of Pure Applied Chemistry (IUPAC) 2013 Recommendations.

In the compounds disclosed herein, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds disclosed herein and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound disclosed herein is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol. 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound disclosed herein is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound disclosed herein simultaneously contain acidic and basic groups in the molecule, the disclosure also includes, in addition to the salt forms mentioned, inner salts or betaines (zwitterions). Salts can be obtained from the compounds disclosed herein by customary methods which are known to the person skilled in the art, for example, by combination with an organic or inorganic acid or base in a solvent or dispersant, or by anion exchange or cation exchange from other salts. The present disclosure also includes all salts of the compounds disclosed herein which, owing to low physiological compatibility, are not directly suitable for use in pharmaceuticals but which can be used, for example, as intermediates for chemical reactions or for the preparation of pharmaceutically acceptable salts.

Furthermore, the compounds disclosed herein may exist in amorphous form and/or one or more crystalline forms, and as such all amorphous and crystalline forms and mixtures thereof of the compounds disclosed herein, including the Examples, are intended to be included within the scope of the present disclosure. In addition, some of the compounds disclosed herein may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound disclosed herein which results in conversion in vivo to a compound within the scope of this disclosure is also within the scope of this disclosure.

The present disclosure also relates to processes for the preparation of the compounds disclosed herein which are described in the following and by which the compounds of the disclosure are obtainable.

The terms “therapeutically effective (or efficacious) amount” and similar descriptions such as “an amount efficacious for treatment” or “an effective dose” are intended to mean that amount of a compound disclosed herein that will elicit the biological or medical response of a tissue, a system, animal or human that is being sought by a researcher, veterinarian, medical doctor or other clinician. In a preferred embodiment, the term “therapeutically effective amount” means an amount of a compound disclosed herein that alleviates at least one clinical symptom in a human patient. The terms “prophylactically effective (or efficacious) amount” and similar descriptions such as “an amount efficacious for prevention” are intended to mean that amount of a compound disclosed herein that will prevent or reduce the risk of occurrence of the biological or medical event that is sought to be prevented in a tissue, a system, animal or human by a researcher, veterinarian, medical doctor or other clinician.

### Dosages of the Compounds Disclosed Herein

The dosage regimen utilizing a compound disclosed herein is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds disclosed herein is within the skill of the art. For administration to a human in, for example, the curative or prophylactic treatment of the conditions and disorders identified herein, the typical dosages of the compounds disclosed herein can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg×5 kg) to about 5000 mg/day (50 mg/kg×100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds disclosed herein and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds disclosed herein and their pharmaceutically acceptable salts for use as pharmaceuticals, their use for modulating the activity of mutant KRAS. HRAS and/or NRAS proteins and in particular their use in the therapy and prophylaxis of the below-mentioned diseases or disorders as well as their use for preparing medicaments for these purposes. In certain embodiments, the compounds disclosed herein and their pharmaceutically acceptable salts inhibit the KRAS G12C protein.

Furthermore, the present disclosure provides pharmaceutical compositions which comprise as active component an effective dose of at least one compound disclosed herein and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound disclosed herein and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound disclosed herein and/or its pharmaceutically acceptable salts in the pharmaceutical composition is from 0.01 to 10 mg per dose. The pharmaceutical compositions usually comprise 0.5 to 90 percent by weight of at least one compound disclosed herein and/or its pharmaceutically acceptable salts. The preparation of the pharmaceutical compositions can be carried out in a manner known per se. For this purpose, one or more compounds disclosed herein and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds disclosed herein and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds Disclosed Herein

The present application provides a method of inhibiting RAS-mediated cell signaling comprising contacting a cell with a compound disclosed herein or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds disclosed herein (or their pharmaceutically acceptable salts) or pharmaceutical compositions containing such compounds to treat disease conditions, including but not limited to, conditions implicated by mutant KRAS. HRAS and/or NRAS proteins (e.g., cancer), and in some embodiments the KRAS G12C mutant.

In some embodiments, a method for treatment of cancer is provided, the method comprising administering a therapeutically effective amount a compound disclosed herein (or a pharmaceutically acceptable salt thereof) or any of the foregoing pharmaceutical compositions comprising such a compound to a subject in need of such treatment. In some embodiments, the cancer is mediated by a KRAS. HRAS or NRAS mutation, e.g., the KRAS G12C mutation. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS, HRAS or NRAS mutation (e.g., KRAS G12C mutation) and if the subject is determined to have the KRAS. HRAS or NRAS mutation, then administering to the subject a therapeutically effective amount of a compound disclosed herein or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound disclosed herein.

KRAS, HRAS or NRAS mutations have also been identified in hematological malignancies (e.g., cancers that affect blood, bone marrow and/or lymph nodes). Accordingly, certain embodiments are directed to administration of the compounds disclosed herein (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkins lymphoma or non-Hodgkins lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS, HRAS or NRAS mutation (e.g., the KRAS G12C mutation) can be undertaken by assessing the nucleotide sequence encoding the KRAS. HRAS or NRAS protein, by assessing the amino acid sequence of the KRAS, HRAS or NRAS protein, or by assessing the characteristics of a putative KRAS. HRAS or NRAS mutant protein. The sequences of wild-type human KRAS, HRAS or NRAS are known in the art.

Methods for detecting a mutation in a KRAS. HRAS or NRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays. PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS, HRAS or NRAS mutations (e.g., the KRAS G12C mutation) by real-time PCR. In real-time PCR, fluorescent probes specific for the KRAS. HRAS or NRAS mutation are used. When a mutation is present, the probe binds and fluorescence is detected. In some embodiments, the KRAS. HRAS or NRAS mutation is identified using a direct sequencing method of specific regions (e.g., exon 2 and/or exon 3) in the KRAS, HRAS or NRAS gene.

Methods for detecting a mutation in a KRAS, HRAS or NRAS protein (e.g., the KRAS G12C mutation) are known by those of skill in the art. These methods include, but are not limited to, detection of a KRAS. HRAS or NRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS, HRAS or NRAS mutation (e.g., the KRAS G12C mutation). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound disclosed herein, or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors. CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplasia syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds disclosed herein (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds disclosed herein may provide therapeutic benefit for include, but are not limited to, glandular tumors, carcinoid tumors and undifferentiated carcinomas.

The present disclosure also provides methods of modulating a mutant KRAS, HRAS or NRAS protein activity (e.g., activity resulting from the KRAS G12C mutation) by contacting the protein with an effective amount of a compound disclosed herein. Modulation can be inhibiting or activating protein activity. In some embodiments, the present disclosure provides methods of inhibiting protein activity by contacting the mutant KRAS, HRAS or NRAS protein (e.g., KRAS G12C mutant) with an effective amount of a compound disclosed herein in solution. In some embodiments, the present disclosure provides methods of inhibiting the mutant KRAS, HRAS or NRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound disclosed herein.

### Combination Therapies

One or more additional pharmacologically active agents may be administered in combination with a compound disclosed herein (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound disclosed herein. The additional active agents also include free-acid, free-base and pharmaceutically acceptable salts of said additional active agents. Generally, any suitable additional active agent or agents, including chemotherapeutic agents or therapeutic antibodies, may be used in any combination with the compound disclosed herein in a single dosage formulation (e.g., a fixed dose drug combination), or in one or more separate dosage formulations which allows for concurrent or sequential administration of the active agents (co-administration of the separate active agents) to subjects. In addition, the compounds disclosed herein (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound disclosed herein, or a pharmaceutically acceptable salt thereof. In one embodiment, such therapy includes, but is not limited to, the combination of one or more compounds disclosed herein with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAG1 agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU). 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan. SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles. EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen. Maruyama vaccine, melanoma lysate vaccine, melanoma oncolysate vaccine (New York Medical College), melanoma vaccine (New York University), melanoma vaccine (Sloan Kettering Institute). TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin. CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilimumab; anti-LAG1 agents; and anti-OX40 agents. Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors. EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors. ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993. Br. J. Cancer 67:247-253; Teramoto, T., et al., 1996. Cancer 77:639-645; Goldstein et al. 1995, Clin. Cancer Res. 1: 1311-1318; Huang, S. M., et al., 1999. Cancer Res. 15:59(8): 1935-40; and Yang, X., et al., 1999. Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM disintegrin domain to antagonize the binding of integrin to its ligands (US 2002/0042368); anti-eph receptor and/or anti-ephrin antibodies or antigen binding regions (U.S. Pat. No. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors. MMP-9 (matrix-metalloproteinase 9) inhibitors, prinomastat. RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4. MMP-5, MMP-6, MMP-7, MMP-8, MMP-10. MMP-11. MMP-12, and MMP-13).

Non-limiting examples of VEGF and VEGFR inhibitory agents include bevacizumab, cediranib, CEP 7055. CP 547632. KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol. AE 941, alemtuzumab, alpha-D148 Mab (Amgen. US), alphastatin, anecortave acetate, angiocidin, angiogenesis inhibitors, (SUGEN, US), angiostatin, anti-Vn Mab (Crucell, Netherlands), atiprimod, axitinib, AZD 9935. BAY RES 2690 (Bayer. Germany, BC 1 (Genoa Institute of Cancer Research. Italy), beloranib, benefin (Lane Labs, US), cabozantinib. CDP 791 (Celltech Group, UK), chondroitinase AC, cilengitide, combretastatin A4 prodrug. CP 564959 (OSI, US). CV247, CYC 381 (Harvard University, US), E 7820, EHT 0101, endostatin, enzastaurin hydrochloride, ER-68203-0 (IVAX, US), fibrinogen-E fragment. Flk-1 (ImClone Systems, US), forms of FLT 1 (VEGFR 1). FR-111142, GCS-100, GW 2286 (GlaxoSmithKline, UK), IL-8, ilomastat, IM-862, irsogladine. KM-2550 (Kyowa Hakko. Japan), lenalidomide, lenvatinib. MAb alpha5beta3 integrin, second generation (Applied Molecular Evolution. USA and MedImmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego. US). SG 292 (Telios, US). SU-0879 (Pfizer. US), TAN-1120. TBC-1635, tesevatinib, tetrathiomolybdate, thalidomide, thrombospondin 1 inhibitor, Tie-2 ligands (Regeneron. US), tissue factor pathway inhibitors (EntreMed. US), tumor necrosis factor-alpha inhibitors, tumstatin, TZ 93, urokinase plasminogen activator inhibitors, vadimezan, vandetanib, vasostatin, vatalanib. VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor. EGFR inhibitor, MEK inhibitor, ERK inhibitor. PI3K inhibitor, AKT inhibitor, TOR inhibitor. MCL-1 inhibitor, BCL-2 inhibitor. SHP2 inhibitor, proteasome inhibitor, or immune therapy, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901. PD334581, PD98059, refametinib, selumetinib, and trametinib.

Non-limiting examples of ERK inhibitors include LY3214996, LTT462. MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; 1C87114; idelalisib; INK1117; LY294002; Palomid 529; paxalisib; perifosine; PI-103; PI-103 hydrochloride; pictilisib (e.g., WO 09/036,082; WO 09/055,730); P1K 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Aktl) (Barnett et al. (2005) Biochem. J., 385 (Pt. 2). 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl). 3493S-3498S); perifosine, Dasmahapatra et al. (2004) Clin. Cancer Res. 10(15). 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus. WO 9409010; rapalogs, e.g. as disclosed in WO 98/02441 and WO 01/14387. e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. No. 5,118,677. U.S. Pat. Nos. 5,118,678, 5,100,883, 5,151,413. U.S. Pat. No. 5,120,842. WO 93/111130. WO 94/02136, WO 94/02485, WO 95/14023. WO 94/02136. WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176. MIK665, and S63845. Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide. 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine. BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate. DA 3030 (Dong-A), defofamine, denileukin diftitox, dexrazoxane, diaziquone, dichloroacetic acid, dilazep, discodermolide, docosanol, doxercalciferol, edelfosine, eflomithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin 1 (Harvard University, US), urethan, valspodar, verteporfin, zoledronic acid, and zosuquidar.

The present disclosure further provides a method for using the compounds disclosed herein or pharmaceutical compositions provided herein, in combination with radiation therapy to treat cancer. Techniques for administering radiation therapy are known in the art, and these techniques can be used in the combination therapy described herein. The administration of the compound disclosed herein in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy.” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source. I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198. Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound disclosed herein, or a pharmaceutically acceptable salt thereof. In one embodiment, such therapy includes, but is not limited to, the combination of one or more compounds disclosed herein with chemotherapeutic agents, immunotherapeutic agents, hormonal therapy agents, therapeutic antibodies, targeted therapy agents, and radiation treatment, to provide a synergistic or additive therapeutic effect. The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound disclosed herein and any of the agents described above can be formulated together in the same dosage form and administered simultaneously. Alternatively, a compound disclosed herein and any of the agents described above can be simultaneously administered, wherein both the agents are present in separate formulations. In another alternative, a compound disclosed herein can be administered just followed by and any of the agents described above, or vice versa. In some embodiments of the separate administration protocol, a compound disclosed herein and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound disclosed herein, and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound disclosed herein, or the pharmaceutically acceptable salt thereof, for use in therapy, or use of the compound disclosed herein, or the pharmaceutically acceptable salt thereof, in therapy. The present disclosure also provides for the compound disclosed herein, or the pharmaceutically acceptable salt thereof, for use in treating cancer, or use of a compound disclosed herein, or the pharmaceutically acceptable salt thereof, for treating cancer. The present disclosure also provides for the compound disclosed herein, or the pharmaceutically acceptable salt thereof, for the preparation of a medicament for the treatment of cancer, or use of the compound disclosed herein, or the pharmaceutically acceptable salt thereof, for the preparation of a medicament for the treatment of cancer. The present disclosure also provides for the compound disclosed herein, or the pharmaceutically acceptable salt thereof, and an additional anti-cancer agent, for use in the treatment of cancer, or use of the compound disclosed herein, or the pharmaceutically acceptable salt thereof, and the additional anti-cancer agent for treating cancer. The disclosure also provides the compound disclosed herein, or the pharmaceutically acceptable salt thereof, and an additional anti-cancer agent, for the preparation of a medicament for the treatment of cancer, or use of the compound disclosed herein, or the pharmaceutically acceptable salt thereof, and the additional anti-cancer agent, for the preparation of a medicament for the treatment of cancer. The present disclosure also provides for a pharmaceutical composition comprising the compound disclosed herein, or the pharmaceutically acceptable salt thereof, for use in the treatment of cancer, or use of the pharmaceutical composition comprising the compound disclosed herein, or the pharmaceutically acceptable salt thereof, for treating cancer. The present disclosure also provides for a pharmaceutical composition comprising the compound disclosed herein, or the pharmaceutically acceptable salt thereof, and an additional anti-cancer agent, for use in the treatment of cancer, or use of the pharmaceutical composition comprising the compound disclosed herein, or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Throughout the synthetic schemes and examples, abbreviations and acronyms may be used with the following meanings unless otherwise indicated: s: singlet, d: doublet, t: triplet, q: quartet, sep: septet, dd: double doublet, dt: double triplet, td: triple doublet, tt: triple triplet, ddd: double double doublet, ddt: double double triplet, dtd: double triple doublet, tdd: triple double doublet. m: multiplet. br: broad, brs: broad singlet, tert: tertiary, DMSO-d₆: deuterated dimethyl sulfoxide, CDCl₃: deuterated chloroform, CD₃OD: deuterated methanol. THF: tetrahydrofuran. DMF: N,N-dimethylformamide, NMP: 1-methyl-2-pyrrolidinone, DMSO: dimethyl sulfoxide. DMA: N,N-dimethyllactamide, DME: 1,2-dimethoxyethane, DCM: dichloromethane. IPE: diisopropyl ether, MTBE: methyl tert-butyl ether, EtOAc: ethyl acetate. AcOH: acetic acid, MeOH: methanol. EtOH: ethanol, iPrOH: isopropanol. T3P: propylphosphonic anhydride. Et₃N: triethylamine, RT: room temperature. NBS: N-bromosuccinimide, NIS: N-iodosuccinimide, Ms: methanesulfonyl. TIPS: triisopropylsilyl, TBAF: tetrabutylammonium fluoride, Pd(dppf)Cl₂: [1,1′-Bis(diphenylphosphino)ferrocene]dichloropalladium(II), and JohnPhos: (2-Biphenylyl)di-tert-butylphosphine.

The reagents used in the Examples are commercially available products unless indicated otherwise. Prepacked columns manufactured by Shoko Scientific Co., Ltd., or Biotage were used in silica gel column chromatography and basic silica gel column chromatography. An AVANCE NEO 400 spectrometer (400 MHz; BRUKER) was used for NMR spectra. For a deuterated solvent containing tetramethylsilane, tetramethylsilane was used as the internal reference. For other cases, measurement was performed using an NMR solvent as the internal reference. All S values are indicated in ppm. Microwave reaction was performed using an Initiator (trademark) manufactured by Biotage. XSelect CSH C18 OBD Prep Columns manufactured by Waters were used for preparative reversed-phase HPLC.

### Example 1

### (E)-N-(2,6-difluoro-4-(8-(6-methoxy-1-methyl-1H-benzo[d]imidazol-5-yl)indolizine-3-carbonyl)phenyl)-4-(((1r,4r)-4-methoxycyclohexyl)amino)but-2-enamide

### Step 1

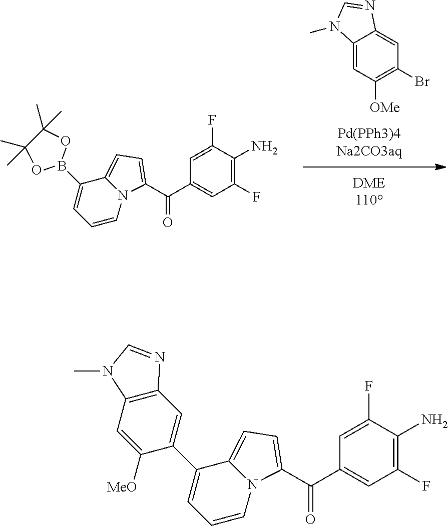 [SMILES: CC1(C)OB(c2cccn3c(C(=O)c4cc(F)c(N)c(F)c4)ccc23)OC1(C)C ; COc1cc2c(cc1Br)ncn2C ; COc1cc2c(cc1-c1cccn3c(C(=O)c4cc(F)c(N)c(F)c4)ccc13)ncn2C]

To a mixture of (4-amino-3,5-difluorophenyl)(8-(4,4,5,5-tetramethyl-1,3,2-dioxaborolan-2-yl)indolizin-3-yl)methanone (153 mg, 0.384 mmol). 5-bromo-6-methoxy-1-methyl-1H-benzo[d]imidazole (130 mg, 0.384 mmol) and Pd(PPh₃)₄ (44 mg) in 1,2-dimethoxyethane (1.0 mL) was added 2M aqueous sodium carbonate solution (0.384 mL). After reacting by microwave apparatus at 110° C. for 2 hours, the mixture was diluted with EtOAc and water. The organic layer was washed with brine, dried over sodium sulfate and evaporated under reduced pressure. The residue was purified by column chromatography on silica gel (CHCl₃-ethanol) to give (4-amino-3,5-difluorophenyl)(8-(6-methoxy-1-methyl-1H-benzo[d]imidazol-5-yl)indolizin-3-yl)methanone (61.2 mg).

### Step 2

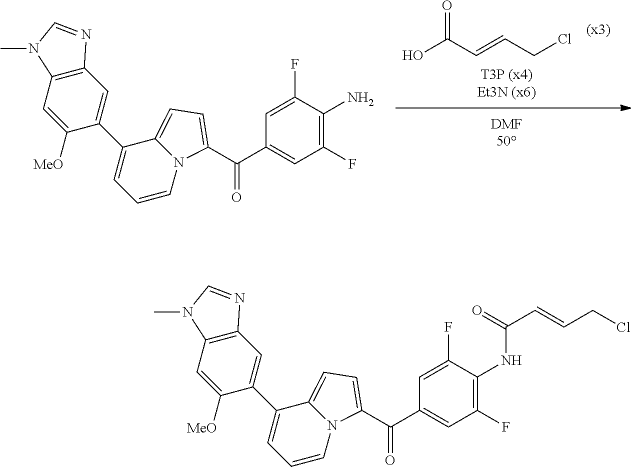 [SMILES: COc1cc2c(cc1-c1cccn3c(C(=O)c4cc(F)c(N)c(F)c4)ccc13)ncn2C ; O=C(O)/C=C/CCl ; COc1cc2c(cc1-c1cccn3c(C(=O)c4cc(F)c(NC(=O)/C=C/CCl)c(F)c4)ccc13)ncn2C]

A solution of (4-amino-3,5-difluorophenyl)(8-(6-methoxy-1-methyl-1H-benzo[d]imidazol-5-yl)indolizin-3-yl)methanone (61 mg, 0.141 mmol). 4-chlorocrotonic acid (51 mg, 0.423 mmol) and propylphosphonic anhydride solution (50 wt % in THF, 1.4 mL, 0.563 mmol) in DMF (1.0 mL) was heated at 50° C. for 30 min. Then triethylamine (0.118 mL, 0.847 mmol) was added thereto, and the mixture was stirred at 50° C. for 30 min. To the resulting mixture was added saturated aqueous NaHCO₃, and extracted with EtOAc. and washed successively with H₂O then brine, dried over sodium sulfate, and evaporated. The residue was purified by column chromatography on silica gel (gradient elution, CHCl₃-ethanol) to give (E)-4-chloro-N-(2,6-difluoro-4-(8-(6-methoxy-1-methyl-1H-benzo[d]imidazol-5-yl)indolizine-3-carbonyl)phenyl)but-2-enamide.

### Step 3

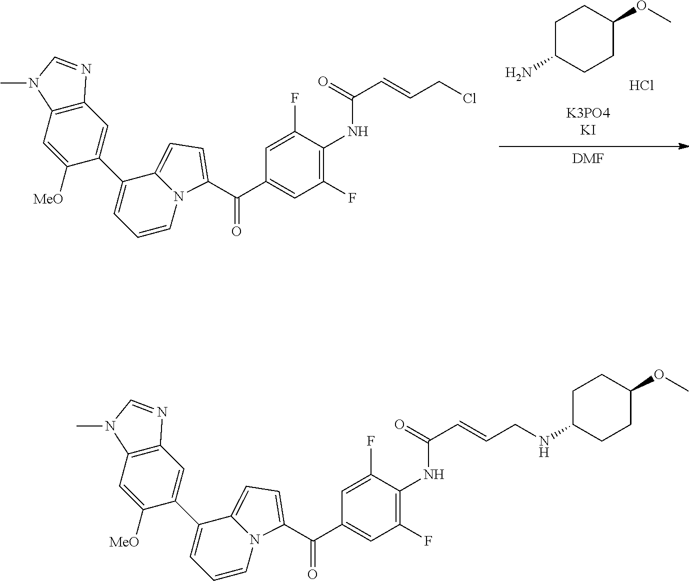

A solution of the above (E)-4-chloro-N-(2,6-difluoro-4-(8-(6-methoxy-1-methyl-1H-benzo[d]imidazol-5-yl)indolizine-3-carbonyl)phenyl)but-2-enamide, trans-4-methoxycyclohexanamine hydrochloride (70 mg, 0.423 mmol). KI (70 mg, 0.422 mmol) and K3PO4 (70 mg, 0.330 mmol) in DMF (1.0 mL) was stirred at 50° C. for 1.0 hour. To the resulting mixture was added saturated aqueous NaHCO₃, and extracted with EtOAc. and washed successively with H2O then brine, dried over sodium sulfate, and evaporated. The residue was purified by preparative HPLC (water:acetonitrile (0.1% formic acid)) to give the title compound (30.1 mg). f

### Example 2

### (E)-N-(4-(8-(1,2-dimethyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide

### Step 1

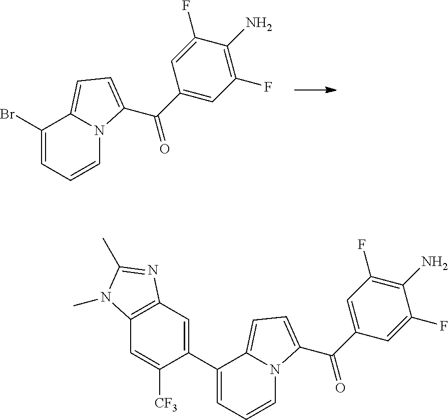 [SMILES: Nc1c(F)cc(C(=O)c2ccc3c(Br)cccn23)cc1F ; Cc1nc2cc(-c3cccn4c(C(=O)c5cc(F)c(N)c(F)c5)ccc34)c(C(F)(F)F)cc2n1C]

A solution of (4-amino-3,5-difluorophenyl)(8-bromoindolizin-3-yl)methanone (50 mg, 0.14 mmol), 1,2-dimethyl-5-(4,4,5,5-tetramethyl-1,3,2-dioxaborolan-2-yl)-6-(trifluoromethyl)-1H-benzo[d]imidazole (73 mg, 0.21 mmol). Pd(PPh₃)₂Cl₂ (10 mg, 0.014 mmol) and 2M Na₂CO₃ aq. (0.14 mL) in DMF (1.0 mL) was evacuated and N₂ back-filled. After reacting by microwave apparatus at 110° C. for 2 hours, the mixture was diluted with EtOAc and water. The organic layer was washed with H₂O then brine, dried over sodium sulfate and evaporated under reduced pressure. The residue was purified by column chromatography on silica gel (CHCl₃-methanol) to give crude (4-amino-3,5-difluorophenyl)(8-(1,2-dimethyl-6-(trifluoromethyl)-1H-benzo[d]imidazol-5-yl)indolizin-3-yl)methanone (100 mg).

### Step 2

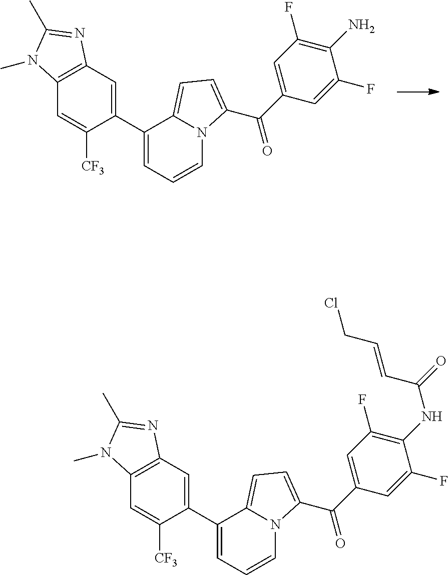 [SMILES: Cc1nc2cc(-c3cccn4c(C(=O)c5cc(F)c(N)c(F)c5)ccc34)c(C(F)(F)F)cc2n1C ; Cc1nc2cc(-c3cccn4c(C(=O)c5cc(F)c(NC(=O)/C=C/CCl)c(F)c5)ccc34)c(C(F)(F)F)cc2n1C]

A solution of crude (4-amino-3,5-difluorophenyl)(8-(1,2-dimethyl-6-(trifluoromethyl)-1H-benzo[d]imidazol-5-yl)indolizin-3-yl)methanone (100 mg), 4-chlorocrotonic acid (51 mg, 0.42 mmol) and propylphosphonic anhydride solution (50 wt % in THF. 0.27 mL, 0.42 mmol) in DMF (1.0 mL) was stirred at RT for 15 min. Then triethylamine (0.097 mL, 0.70 mmol) was added thereto, and the mixture was stirred at RT for 4 hours. To the resulting mixture was added saturated aqueous NaHCO₃ and water, and extracted with EtOAc. and the organic layer was washed successively with H₂O, saturated aqueous NaHCO₃ then brine, dried over sodium sulfate, and evaporated to give crude (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (82 mg).

### Step 3

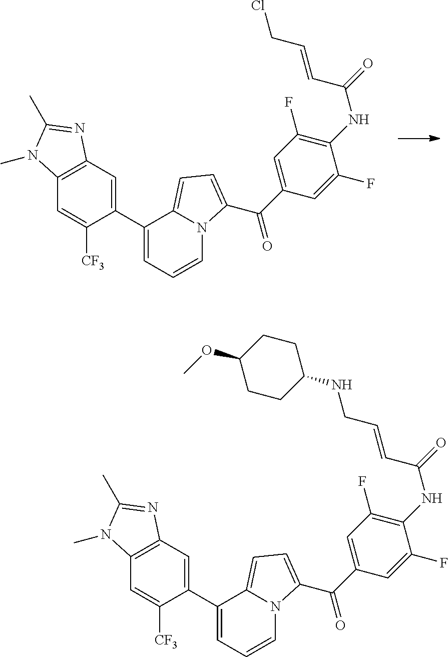 [SMILES: Cc1nc2cc(-c3cccn4c(C(=O)c5cc(F)c(NC(=O)/C=C/CCl)c(F)c5)ccc34)c(C(F)(F)F)cc2n1C ; CO[C@H]1CC[C@H](NC/C=C/C(=O)Nc2c(F)cc(C(=O)c3ccc4c(-c5cc6nc(C)n(C)c6cc5C(F)(F)F)cccn34)cc2F)CC1]

A solution of crude (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (82 mg), trans-4-methoxycyclohexanamine hydrochloride (58 mg, 0.35 mmol). KI (70 mg, 0.42 mmol) and K₃PO₄ (150 mg, 0.70 mmol) in DMF (1.0 mL) was stirred at 50° C. for 50 min. To the resulting mixture was added H₂O, and extracted with EtOAc, and the organic layer was washed successively with H₂O then brine, dried over sodium sulfate, and evaporated. The residue was purified by preparative HPLC (water:acetonitrile (0.1% formic acid)). Saturated sodium bicarbonate water and H₂O were added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (67 mg).

### Example 3

### (E)-N-(4-(8-(2-ethyl-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide

### Step 1

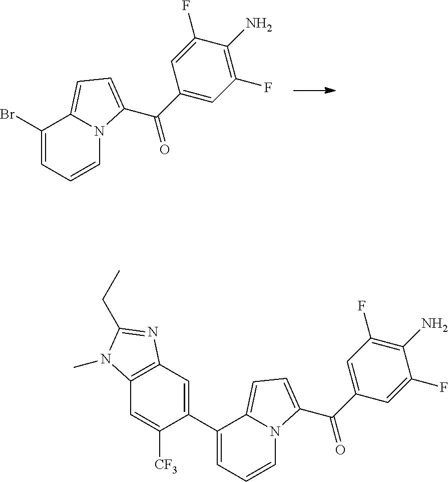 [SMILES: Nc1c(F)cc(C(=O)c2ccc3c(Br)cccn23)cc1F ; CCc1nc2cc(-c3cccn4c(C(=O)c5cc(F)c(N)c(F)c5)ccc34)c(C(F)(F)F)cc2n1C]

A solution of (4-amino-3,5-difluorophenyl)(8-bromoindolizin-3-yl)methanone (50 mg, 0.14 mmol), 2-ethyl-1-methyl-5-(4,4,5,5-tetramethyl-1,3,2-dioxaborolan-2-yl)-6-(trifluoromethyl)-1H-benzo[d]imidazole (95 mg, 0.21 mmol). Pd(PPh₃)₂Cl₂ (10 mg, 0.014 mmol) and 2M Na₂CO₃ aq. (0.14 mL) in DMF (1.0 mL) was evacuated and N₂ back-filled. After reacting by microwave apparatus at 110° C. for 2 hours, the mixture was diluted with EtOAc and water. The organic layer was washed with H₂O then brine, dried over sodium sulfate and evaporated under reduced pressure. The residue was suspended in IPE (1 mL) and the suspension was stirred at RT for 90 min. The precipitate was collected via filtration, washing with IPE, and the solid dried in vacuo to give (4-amino-3,5-difluorophenyl)(8-(2-ethyl-1-methyl-6-(trifluoromethyl)-1H-benzo[d]imidazol-5-yl)indolizin-3-yl)methanone (62 mg).

### Step 2

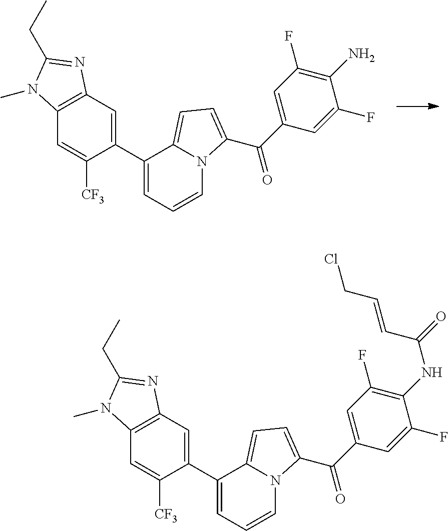 [SMILES: CCc1nc2cc(-c3cccn4c(C(=O)c5cc(F)c(N)c(F)c5)ccc34)c(C(F)(F)F)cc2n1C ; CCc1nc2cc(-c3cccn4c(C(=O)c5cc(F)c(NC(=O)/C=C/CCl)c(F)c5)ccc34)c(C(F)(F)F)cc2n1C]

The procedure of Example 2 (step 2) was performed except that (4-amino-3,5-difluorophenyl)(8-(2-ethyl-1-methyl-6-(trifluoromethyl)-1H-benzo[d]imidazol-5-yl)indolizin-3-yl)methanone (62 mg) obtained in step 1 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2) thereby obtaining crude (E)-4-chloro-N-(4-(8-(2-ethyl-1-methyl-6-(trifluoromethyl)-1H-benzo[d]imidazol-5-yl)indolizine-3-carbonyl)-2,6-difluorophenyl)but-2-enamide (72 mg).

### Step 3

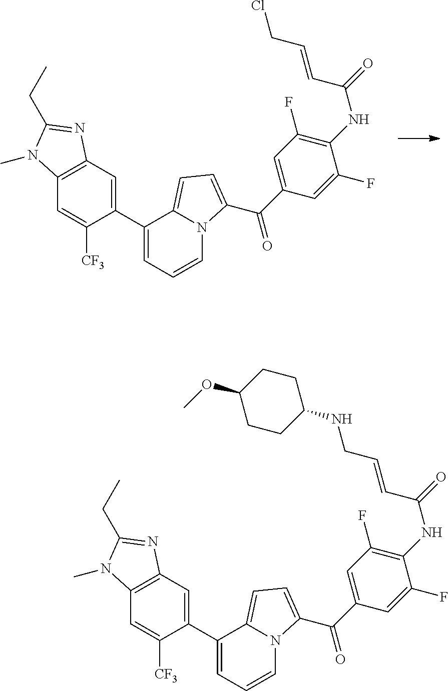 [SMILES: CCc1nc2cc(-c3cccn4c(C(=O)c5cc(F)c(NC(=O)/C=C/CCl)c(F)c5)ccc34)c(C(F)(F)F)cc2n1C ; CCc1nc2cc(-c3cccn4c(C(=O)c5cc(F)c(NC(=O)/C=C/CN[C@H]6CC[C@H](OC)CC6)c(F)c5)ccc34)c(C(F)(F)F)cc2n1C]

The procedure of Example 2 (step 3) was performed except that crude (E)-4-chloro-N-(4-(8-(2-ethyl-1-methyl-6-(trifluoromethyl)-1H-benzo[d]imidazol-5-yl)indolizine-3-carbonyl)-2,6-difluorophenyl)but-2-enamide (72 mg) obtained in step 2 was used instead of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide in Example 2 (step 3), thereby obtaining the title compound (55 mg).

### Example 4

### (E)-N-(2,6-difluoro-4-(8-(6-methoxy-1,2-dimethyl-1H-benzo[d]imidazol-5-yl)indolizine-3-carbonyl)phenyl)-4-(((1r,4r)-4-methoxycyclohexyl)amino)but-2-enamide

### Step 1

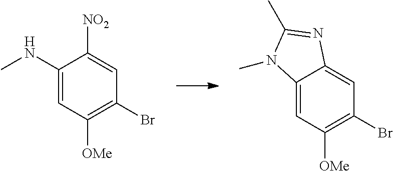

The mixture of 4-bromo-5-methoxy-N-methyl-2-nitroaniline (2.61 g, 10 mmol), ammonium chloride (2.67 g. 50 mmol) and zinc dust (3.27 g. 50 mmol) in ethanol (10 mL) and water (5.0 mL) was stirred at 80° C. overnight. To the resulting mixture was added 28% aqueous ammonium hydroxide solution and EtOAc, and the organic layer was separated, and washed with brine, dried over sodium sulfate, and evaporated. To the residue was added trimethyl orthoacetate (18 mL) and slowly added concentrated aqueous HCl (1 mL). The mixture was diluted with water, and neutralized by aqueous NaOH, and extracted with EtOAc. The organic layer was washed with brine, dried over sodium sulfate, and evaporated. The residue was purified by column chromatography on silica gel (gradient elution, hexane-EtOAc/ethanol(4/1)) to give 5-bromo-6-methoxy-1,2-dimethyl-1H-benzo[d]imidazole (1.08 g).

### Step 2

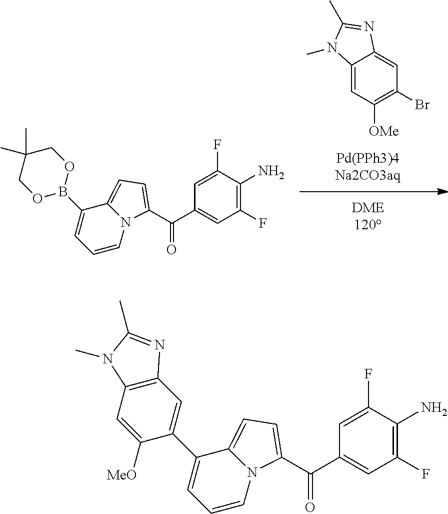 [SMILES: CC1(C)COB(c2cccn3c(C(=O)c4cc(F)c(N)c(F)c4)ccc23)OC1 ; COc1cc2c(cc1Br)nc(C)n2C ; COc1cc2c(cc1-c1cccn3c(C(=O)c4cc(F)c(N)c(F)c4)ccc13)nc(C)n2C]

To a mixture of (4-amino-3,5-difluorophenyl)(8-(5,5-dimethyl-1,3,2-dioxaborinan-2-yl)indolizin-3-yl)methanone (236 mg.), 5-bromo-6-methoxy-1,2-dimethyl-1H-benzo[d]imidazole (130 mg) and Pd(PPh₃)₄ (29 mg) in 1,2-dimethoxyethane (3.0 mL) was added 2M aqueous sodium carbonate solution (0.5 mL). After reacting by microwave apparatus at 120° C. for 3 hours, the mixture was diluted with EtOAc and water. The organic layer was washed with brine, dried over sodium sulfate and evaporated under reduced pressure. The residue was purified by column chromatography on silica gel (CHCl₃-ethanol) to give (4-amino-3,5-difluorophenyl)(8-(6-methoxy-1,2-dimethyl-1H-benzo[d]imidazol-5-yl)indolizin-3-yl)methanone (200 mg).

### Step 3

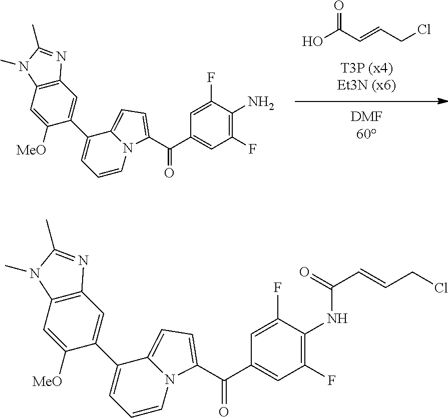 [SMILES: COc1cc2c(cc1-c1cccn3c(C(=O)c4cc(F)c(N)c(F)c4)ccc13)nc(C)n2C ; O=C(O)/C=C/CCl ; COc1cc2c(cc1-c1cccn3c(C(=O)c4cc(F)c(NC(=O)/C=C/CCl)c(F)c4)ccc13)nc(C)n2C]

A solution of (4-amino-3,5-difluorophenyl)(8-(6-methoxy-1,2-dimethyl-1H-benzo[d]imidazol-5-yl)indolizin-3-yl)methanone (200 mg, 0.448 mmol), 4-chlorocrotonic acid (162 mg, 1.34 mmol) and propylphosphonic anhydride solution (50 wt % in THF, 1.4 mL, 2.24 mmol) in DMF (3.00 mL) was heated at 45° C. for 30 min. Then triethylamine (0.400 mL, 2.87 mmol) was added thereto, and the mixture was stirred at 45° C. for 30 min. To the resulting mixture was added saturated aqueous NaHCO₃, and extracted with EtOAc. and washed successively with H₂O then brine, dried over sodium sulfate, and evaporated. The residue was purified by column chromatography on silica gel (gradient elution, CHCl₃-ethanol) to give (E)-4-chloro-N-(2,6-difluoro-4-(8-(6-methoxy-1,2-dimethyl-1H-benzo[d]imidazol-5-yl)indolizine-3-carbonyl)phenyl)but-2-enamide (75.4 mg).

### Step 4

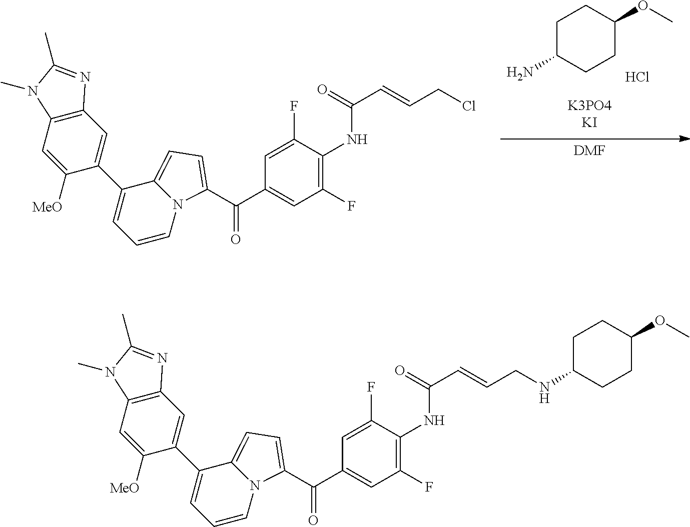

A solution of (E)-4-chloro-N-(2,6-difluoro-4-(8-(6-methoxy-1,2-dimethyl-1H-benzo[d]imidazol-5-yl)indolizine-3-carbonyl)phenyl)but-2-enamide (37 mg, 0.0674 mmol), trans-4-methoxycyclohexanamine hydrochloride (20 mg, 0.120 mmol), KI (20 mg. 0.120 mmol) and K₃PO₄ (72 mg, 0.339 mmol) in DMF (1.0 mL) was stirred at 40° C. for 2.5 hours. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (5.95 mg).

### Example 5

### (E)-4-(tert-butylamino)-N-(2,6-difluoro-4-(8-(6-methoxy-1,2-dimethyl-1H-benzo[d]imidazol-5-yl)indolizine-3-carbonyl)phenyl)but-2-enamide

A solution of (E)-4-chloro-N-(2,6-difluoro-4-(8-(6-methoxy-1,2-dimethyl-1H-benzo[d]imidazol-5-yl)indolizine-3-carbonyl)phenyl)but-2-enamide (37 mg, 0.0674 mmol), tert-butylamine (0.142 μL) and KI (20 mg, 0.120 mmol) in DMF (1.0 mL) was stirred at room temperature for 4 hours. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (27.42 mg).

### Example 6

### (E)-N-(2,6-difluoro-4-(8-(7-(trifluoromethyl)-2,3-dihydro-1H-benzo[d]pyrrolo[1,2-a]imidazol-6-yl)imidazo[1,2-a]pyridine-3-carbonyl)phenyl)-4-(((1r,4r)-4-methoxycyclohexyl)amino)but-2-enamide

### Step 1

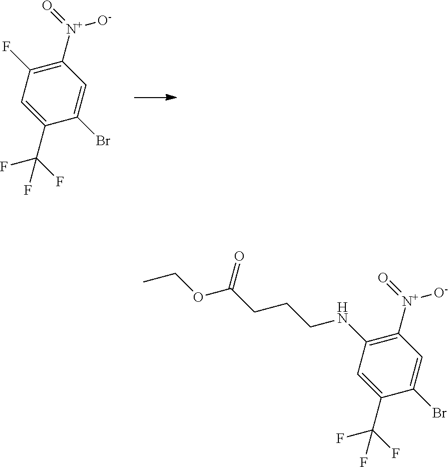 [SMILES: O=[N+]([O-])c1cc(Br)c(C(F)(F)F)cc1F ; CCOC(=O)CCCNc1cc(C(F)(F)F)c(Br)cc1[N+](=O)[O-]]

A solution of 1-bromo-4-fluoro-5-nitro-2-(trifluoromethyl)benzene (2.00 g, 6.94 mmol), ethyl 4-aminobutyrate hydrochloride (1.75 g, 10.4 mmol) and K₂CO₃ (2.88 g, 20.8 mmol) in CH₃CN (14 mL) was stirred at 60° C. for 4 hours After cooling, the reaction mixture was diluted with EtOAc, washed with H₂O and brine then dried over Na₂SO₄ and evaporated. The residue was purified by column chromatography on silica gel (hexane/EtOAc), thereby obtaining ethyl 4-[4-bromo-2-nitro-5-(trifluoromethyl)anilino]butanoate (2.84 g. 102%).

### Step 2

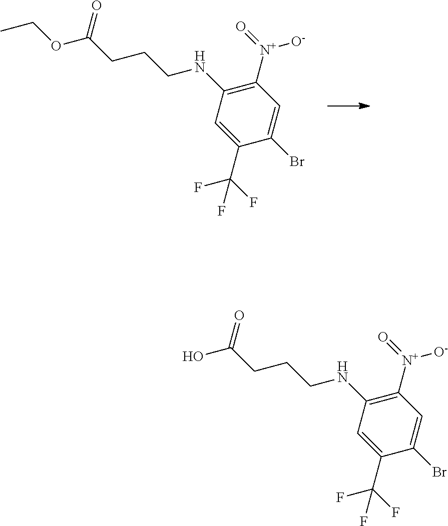 [SMILES: CCOC(=O)CCCNc1cc(C(F)(F)F)c(Br)cc1[N+](=O)[O-] ; O=C(O)CCCNc1cc(C(F)(F)F)c(Br)cc1[N+](=O)[O-]]

5M NaOH aq. (4.15 mL, 20.7 mmol) was added to a solution of ethyl 4-[4-bromo-2-nitro-5-(trifluoromethyl)anilino]butanoate (2.76 g. 6.91 mmol) in THF (13.8 mL) and MeOH (13.8 mL). After stirring at RT for 1 hour, the reaction was quenched with 1M HCl aq. (20.7 mL, 20.7 mmol) at 0° C. then stirred at RT for 1 hour The resulting solid was collected by filtration, washing with further H₂O and vacuum-dried at 50° C. thereby obtaining crude 4-((4-bromo-2-nitro-5-(trifluoromethyl)phenyl)amino)butanoic acid (2.35 g. 92%).

### Step 3

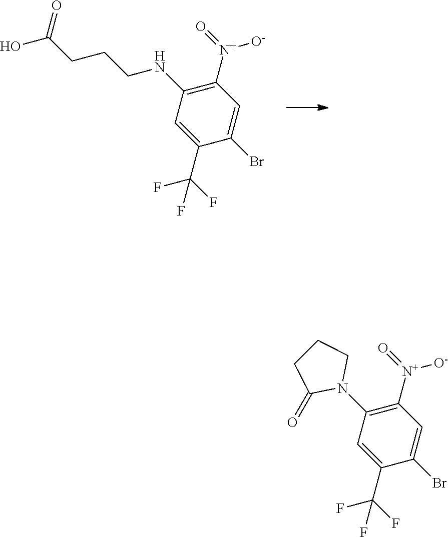 [SMILES: O=C(O)CCCNc1cc(C(F)(F)F)c(Br)cc1[N+](=O)[O-] ; O=C1CCCN1c1cc(C(F)(F)F)c(Br)cc1[N+](=O)[O-]]

Propylphosphonic anhydride solution (50 wt % in THF, 15.9 mL, 25.4 mmol) was added to a solution of 4-((4-bromo-2-nitro-5-(trifluoromethyl)phenyl)amino)butanoic acid (2.35 g, 6.35 mmol). Et₃N (4.40 mL, 31.7 mmol) in THE (42 mL). After stirring at 40° C. for 6 hours, the reaction mixture was diluted with H₂O and sat. NaHCO₃ aq. at 0° C. and evaporated to remove THF. The solution was extracted with EtOAc (2×) then combined organics washed with brine, dried over Na₂SO₄ and evaporated, thereby obtaining crude 1-(4-bromo-2-nitro-5-(trifluoromethyl)phenyl)pyrrolidin-2-one and used in the next step without further purification.

### Step 4

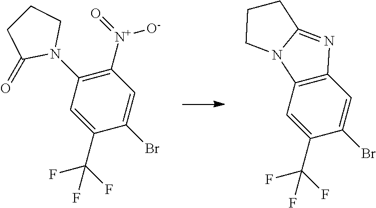 [SMILES: O=C1CCCN1c1cc(C(F)(F)F)c(Br)cc1[N+](=O)[O-] ; FC(F)(F)c1cc2c(cc1Br)nc1n2CCC1]

A solution of crude 1-(4-bromo-2-nitro-5-(trifluoromethyl)phenyl)pyrrolidin-2-one (6.35 mmol) and iron (1.06 g, 19.1 mmol) in AcOH (63.3 mL) was stirred at 120° C. for 20 hours The reaction mixture was evaporated and purified by column chromatography on NH silica gel (hexane/EtOAc), thereby obtaining 6-bromo-7-(trifluoromethyl)-2,3-dihydro-1H-benzo[d]pyrrolo[1,2-a]imidazole (1.59 g, 82%, 2 steps).

### Step 5

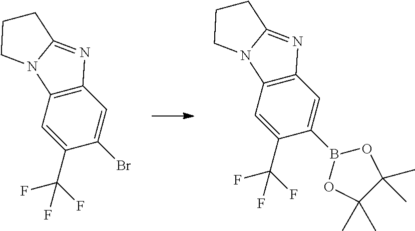 [SMILES: FC(F)(F)c1cc2c(cc1Br)nc1n2CCC1 ; CC1(C)OB(c2cc3nc4n(c3cc2C(F)(F)F)CCC4)OC1(C)C]

A suspension of 6-bromo-7-(trifluoromethyl)-2,3-dihydro-1H-benzo[d]pyrrolo[1,2-a]imidazole (700 mg, 2.29 mmol), AcOK (788 mg, 20.1 mmol), bis(pinacolato)diboron (1.46 g. 5.74 mmol), (1,1′-bis(diphenylphosphino)ferrocene)palladium(II) dichloride dichloromethane adduct (187 mg, 0.229 mmol) in DMSO (7.6 mL) was stirred at 100° C. for 12 hours Further AcOK (158 mg, 0.803 mmol), bis(pinacolato)diboron (291 mg, 1.15 mmol). (1,1′-bis(diphenylphosphino)ferrocene)palladium(II) dichloride dichloromethane adduct (38 mg, 0.046 mmol) were added and stirring continued for 10 hours EtOAc and H₂O were added to the reaction mixture then filtered through a Celite pad. The organic layer was separated and washed with H₂O and brine then dried over Na₂SO₄ and evaporated. The residue was purified by column chromatography on silica gel (hexane/acetone), thereby obtaining 6-(4,4,5,5-tetramethyl-1,3,2-dioxaborolan-2-yl)-7-(trifluoromethyl)-2,3-dihydro-1H-benzo[d]pyrrolo[1,2-a]imidazole (236 mg, 29%).

### Step 6

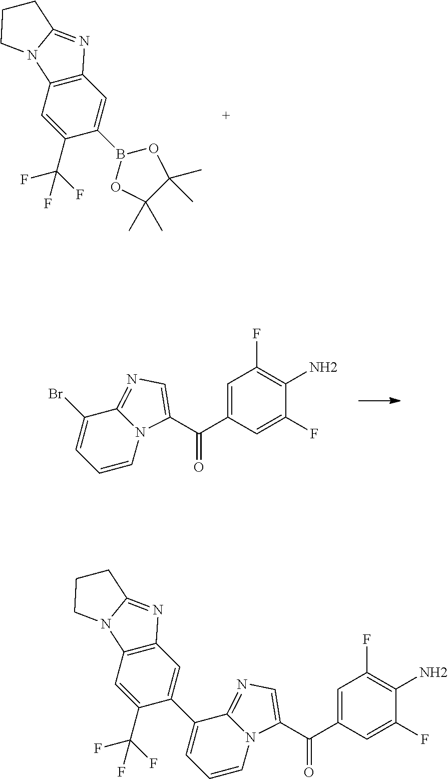 [SMILES: CC1(C)OB(c2cc3nc4n(c3cc2C(F)(F)F)CCC4)OC1(C)C ; Nc1c(F)cc(C(=O)c2cnc3c(Br)cccn23)cc1F ; Nc1c(F)cc(C(=O)c2cnc3c(-c4cc5nc6n(c5cc4C(F)(F)F)CCC6)cccn23)cc1F]

### Step 7

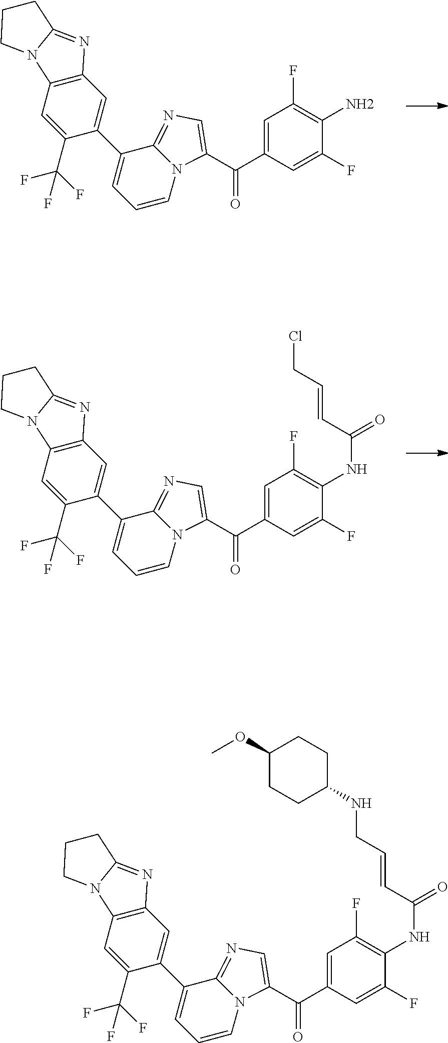 [SMILES: Nc1c(F)cc(C(=O)c2cnc3c(-c4cc5nc6n(c5cc4C(F)(F)F)CCC6)cccn23)cc1F ; O=C(/C=C/CCl)Nc1c(F)cc(C(=O)c2cnc3c(-c4cc5nc6n(c5cc4C(F)(F)F)CCC6)cccn23)cc1F ; CO[C@H]1CC[C@H](NC/C=C/C(=O)Nc2c(F)cc(C(=O)c3cnc4c(-c5cc6nc7n(c6cc5C(F)(F)F)CCC7)cccn34)cc2F)CC1]

The procedure of step 6 and 7 was performed in the same manner as Example 39 (step 1-3) to give the title compound.

### Example 7

### (E)-4-(tert-butylamino)-N-(2,6-difluoro-4-(8-(7-(trifluoromethyl)-2,3-dihydro-1H-benzo[d]pyrrolo[1,2-a]imidazol-6-yl)imidazo[1,2-a]pyridine-3-carbonyl)phenyl)but-2-enamide

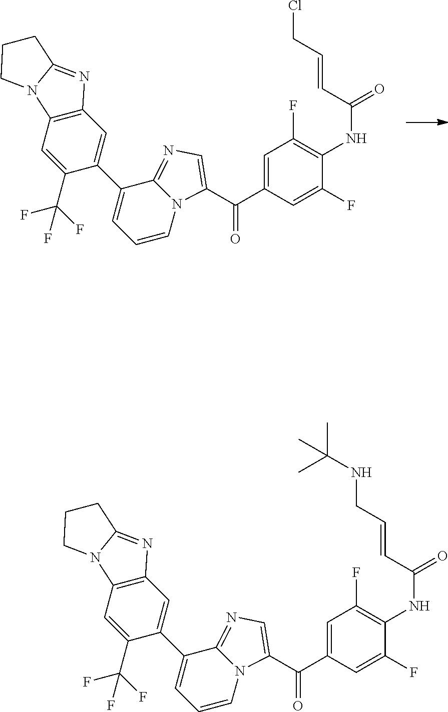 [SMILES: O=C(/C=C/CCl)Nc1c(F)cc(C(=O)c2cnc3c(-c4cc5nc6n(c5cc4C(F)(F)F)CCC6)cccn23)cc1F ; CC(C)(C)NC/C=C/C(=O)Nc1c(F)cc(C(=O)c2cnc3c(-c4cc5nc6n(c5cc4C(F)(F)F)CCC6)cccn23)cc1F]

The procedure was performed in the same manner as Example 40 to give the title compound.

### Example 8

### (E)-N-(2,6-difluoro-4-(8-(1-methyl-6-(trifluoromethyl)-1H-benzo[d]imidazol-5-yl)indolizine-3-carbonyl)phenyl-4-((1-methoxy-2-methylpropan-2-yl)amino)but-2-enamide

### Step 1

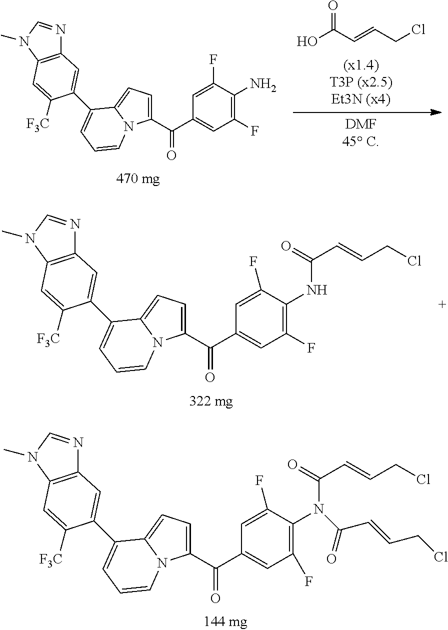 [SMILES: Cn1cnc2cc(-c3cccn4c(C(=O)c5cc(F)c(N)c(F)c5)ccc34)c(C(F)(F)F)cc21 ; O=C(O)/C=C/CCl ; Cn1cnc2cc(-c3cccn4c(C(=O)c5cc(F)c(NC(=O)/C=C/CCl)c(F)c5)ccc34)c(C(F)(F)F)cc21 ; Cn1cnc2cc(-c3cccn4c(C(=O)c5cc(F)c(N(C(=O)/C=C/CCl)C(=O)/C=C/CCl)c(F)c5)ccc34)c(C(F)(F)F)cc21]

The mixture of (4-amino-3,5-difluorophenyl)(8-(1-methyl-6-(trifluoromethyl)-1H-benzo[d]imidazol-5-yl)indolizin-3-yl)methanone (470 mg, 1.0 mmol), 4-chlorocrotonic acid (169 mg, 1.40 mmol) and propylphosphonic anhydride solution (50 wt % in THF. 1.56 mL, 2.50 mmol) in DMF (6.0 mL) was heated at 50° C. for 50 min. Then triethylamine (0.558 mL, 4.00 mmol) was added thereto, and the mixture was stirred at 40° C. for 15 min. To the resulting mixture was added saturated aqueous NaHCO₃, and extracted with EtOAc, and washed successively with H₂O then brine, dried over sodium sulfate, and evaporated. The residue was purified by column chromatography on silica gel (gradient elution, hexane-EtOAc/ethanol(4/1)) to give (E)-4-chloro-N-(2,6-<difluoro-4-(8-(I-methyl-6-(trifluoromethyl)-1H-benzo[d]imidazol-5-yl)indolizine-3-carbonyl)phenyl)but-2-enamide (322 mg) and (E)-4-chloro-N-((E)-4-chlorobut-2-enoyl)-N-(2,6-difluoro-4-(8-(1-methyl-6-(trifluoromethyl)-1H-benzo[d]imidazol-5-yl)indolizine-3-carbonyl)phenyl)but-2-enamide (144 mg).

### Step 2

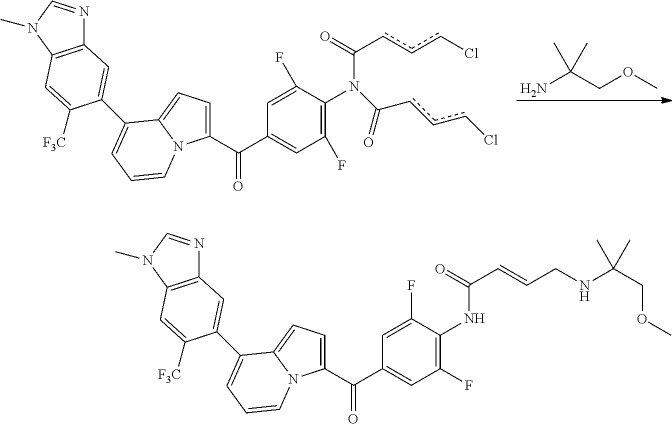

To a solution of the (E)-4-chloro-N-((E)-4-chlorobut-2-enoyl)-N-(2,6-difluoro-4-(8-(1-methyl-6-(trifluoromethyl)-1H-benzo[d]imidazol-5-yl)indolizine-3-carbonyl)phenyl)but-2-enamide (72 mg) in DMF (2 mL) was added 1-methoxy-2-methylpropan-2-amine (0.1 mL). The mixture was stirred at 40° C. for 10 hours. Additional 1-methoxy-2-methylpropan-2-amine (0.05 mL) was added thereto, and stirred at 40° C. overnight. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (23.5 mg).

### Example 9

### (E)-N-(2,6-difluoro-4-(8-(1-methyl-6-(trifluoromethyl)-1H-benzo[d]imidazol-5-yl)indolizine-3-carbonyl)phenyl)-4-((tetrahydro-2H-pyran-4-yl)amino)but-2-enamide

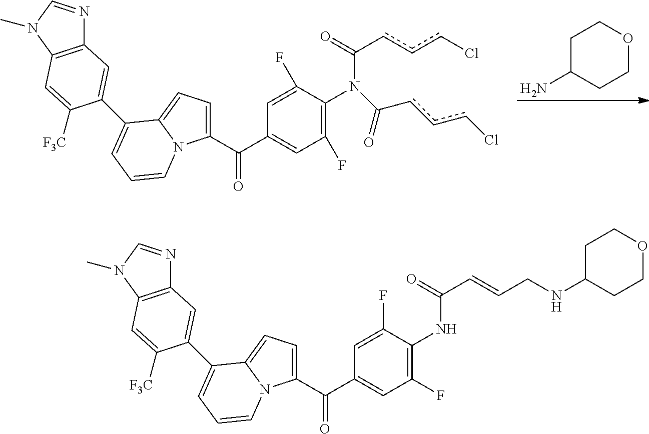

To a solution of the (E)-4-chloro-N-((E)-4-chlorobut-2-enoyl)-N-(2,6-difluoro-4-(8-(1-methyl-6-(trifluoromethyl)-1H-benzo[d]imidazol-5-yl)indolizine-3-carbonyl)phenyl)but-2-enamide (72 mg) in DMF (2 mL) was added tetrahydro-2H-pyran-4-amine (0.1 mL). The mixture was stirred at 40° C. for 4 hours To the resulting mixture was added saturated aqueous NaHCO₃, and the mixture extracted with EtOAc, and washed successively with H₂O then brine, dried over sodium sulfate, and evaporated. The residue was purified by preparative HPLC (water:acetonitrile (0.1% formic acid)) to give the title compound (21.5 mg).

### Example 10

(E)-N-(4-(8-(4-chloro-6-ethyl-1-methyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

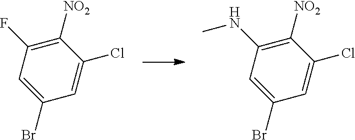

Methylamine (2M THF solution. 7 mL) was added to 5-bromo-1-chloro-3-fluoro-2-nitrobenzene (1.40 g) in THF (3 mL), and the reaction mixture was stirred at 30° C. for 17 hours. After dilution with ethyl acetate, the mixture was washed with water and a saturated sodium chloride solution, followed by drying over sodium sulfate. After evaporation, the residue was purified by column chromatography (hexane:ethyl acetate), thereby obtaining 5-bromo-3-chloro-N-methyl-2-nitroaniline (1.50 g).

### Step 2

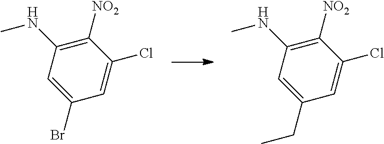

To a solution of 5-bromo-3-chloro-N-methyl-2-nitroaniline (500 mg) obtained in step 1 in 1,2-dimethoxyethane (10 mL), ethylboronic acid (153 mg), tetrakis(triphenylphosphine)palladium(0) (217 mg) and 0.5 M potassium triphosphate in water (7.5 mL) was added. After stirring at 14° C. for 2 hours using microwave irradiation, the mixture was diluted with ethyl acetate and water. After the mixture was extracted with ethyl acetate, the organic layer was washed with a saturated sodium chloride solution, followed by drying over sodium sulfate. The solvent was evaporated under reduced pressure, thereby obtaining crude 3-chloro-5-ethyl-N-methyl-2-nitroaniline (300 mg).

### Step 3

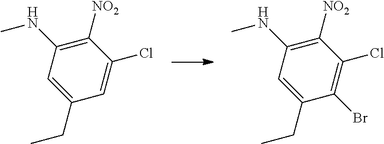

A solution of crude 3-chloro-5-ethyl-N-methyl-2-nitroaniline (300 mg) obtained in step 2 and N-bromosuccinimide (274 mg) in DMF (5 mL) was stirred at room temperature for 30 minutes. After the mixture was diluted with ethyl acetate, the organic layer was washed with a saturated sodium chloride solution, followed by drying over sodium sulfate. After evaporation, the residue was purified by column chromatography (hexane:ethyl acetate), thereby obtaining 4-bromo-3-chloro-5-ethyl-N-methyl-2-nitroaniline (230 mg).

### Step 4

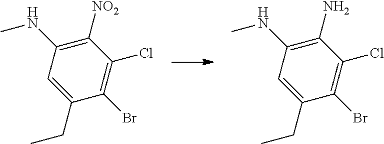

A solution of 4-bromo-3-chloro-5-ethyl-N-methyl-2-nitroaniline (140 mg) obtained in step 3, iron powder (133 mg), and 2 M ammonium chloride solution (1.2 mL) in methanol (2.4 mL) and THF (2.4 mL) was stirred at 75° C. for 3 hours. The insoluble matter was filtered off, followed by evaporating the solvent under reduced pressure. After dilution with ethyl acetate, the mixture was washed with water and a saturated sodium chloride solution, followed by drying over sodium sulfate. The solvent was evaporated under reduced pressure, thereby obtaining crude 4-bromo-3-chloro-5-ethyl-N1-methylbenzene-1,2-diamine (220 mg).

### Step 5

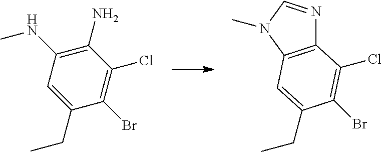

Concentrated hydrochloric acid (25 μL) was added to a solution of crude 4-bromo-3-chloro-5-ethyl-N1-methylbenzene-1,2-diamine (220 mg) obtained in step 4 in triethoxymethane (1.5 mL), followed by stirring at room temperature for 30 minutes. 5M sodium hydroxide solution (128 μL), water, and ethyl acetate were added to the reaction mixture and the aqueous layer was extracted with ethyl acetate. The combined organic layer was washed with a saturated sodium chloride solution, followed by drying over sodium sulfate. After evaporation, the residue was purified by column chromatography (chloroform:ethanol), thereby obtaining 5-bromo-4-chloro-6-ethyl-1-methyl-1H-benzo[d]imidazole (160 mg).

### Step 6

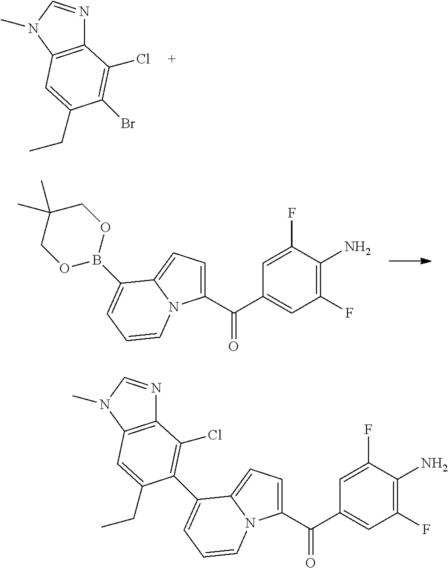

A solution of 5-bromo-4-chloro-6-ethyl-1-methyl-1H-benzo[d]imidazole (27.0 mg) obtained in step 5. (4-amino-3,5-difluorophenyl)(8-(5,5-dimethyl-1,3,2-dioxaborinan-2-yl)indolizin-3-yl)methanone (52.0 mg), tetrakis(triphenylphosphine)palladium(O) (12.5 mg). 2.0 M sodium bicarbonate in water (100 μL) in 1,2-dimethoxyethane (1.0 mL) was stirred at 120° C. for 2 hours using microwave irradiation. After dilution with ethyl acetate and water, the mixture was extracted with ethyl acetate. The combined organic layer was washed with water and a saturated sodium chloride solution, followed by drying over sodium sulfate. The solvent was evaporated under reduced pressure, thereby obtaining crude (4-amino-3,5-difluorophenyl)(8-(4-chloro-6-ethyl-1-methyl-1H-benzo[d]imidazol-5-yl)indolizin-3-yl)methanone (47.0 mg).

### Step 7

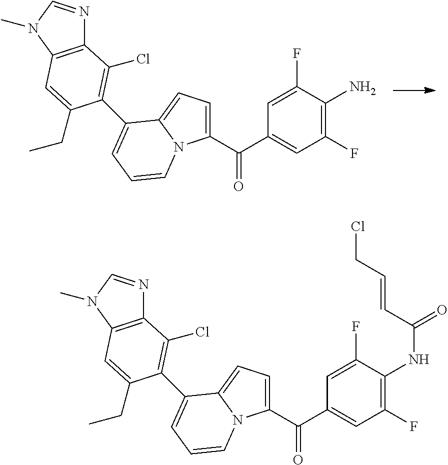 [SMILES: CCc1cc2c(ncn2C)c(Cl)c1-c1cccn2c(C(=O)c3cc(F)c(N)c(F)c3)ccc12 ; CCc1cc2c(ncn2C)c(Cl)c1-c1cccn2c(C(=O)c3cc(F)c(NC(=O)/C=C/CCl)c(F)c3)ccc12]

(E)-4-chlorobut-2-enoic acid (12.2 mg) and 1-propanephosphonic acid anhydride cyclic trimer (a 1.7M THF solution, 200 μL) were added to a solution of (4-amino-3,5-difluorophenyl)(8-(4-chloro-6-ethyl-1-methyl-1H-benzo[d]imidazol-5-yl)indolizin-3-yl)methanone (47.0 mg) obtained in step 6 in DMF (0.8 mL), followed by stirring at room temperature for 20 minutes. Triethylamine (86 μL) was added to the reaction mixture. A reaction was performed at room temperature for 2 hours. After water was added to the reaction mixture, extraction was performed with ethyl acetate, followed by washing the organic layer with a saturated sodium chloride solution. The washed organic layer was dried over sodium sulfate. After evaporation, the obtained residue was purified by column chromatography (chloroform:ethanol), thereby obtaining (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide (58.4 mg).

### Step 8

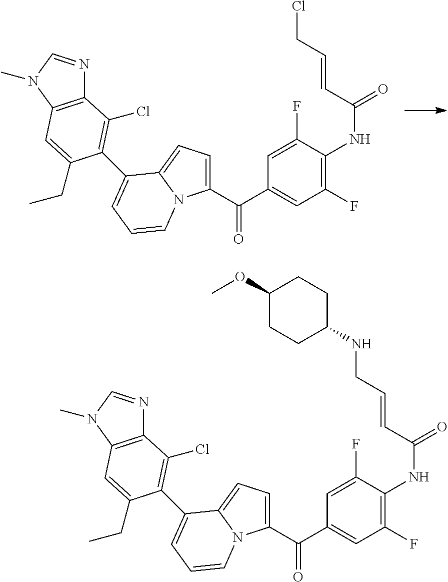

(1r,4r)-4-methoxycyclohexan-1-amine hydrochloride (34.1 mg), tripotassium phosphate (87.5 mg), and potassium iodide (51.3 mg) were added to a solution of (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide (58.4 mg) obtained in step 7 in DMF (500 μL). A reaction was performed at room temperature for 2 hours. After water was added to the reaction mixture, extraction was performed with ethyl acetate. The combined organic layer was washed with a saturated sodium chloride solution. After the washed layer was dried over sodium sulfate, the solvent was evaporated under reduced pressure. The residue was purified by preparative reversed-phase HPLC (water:acetonitrile (0.1% formic acid)). Saturated sodium bicarbonate water was added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (15.1 mg).

### Example 11

(E)-N-(2,6-difluoro-4-(8-(1-methyl-6-(trifluoromethyl)-1H-benzo[d]imidazol-5-yl)indolizine-3-carbonyl)phenyl)-4-((4-methyltetrahydro-2H-pyran-4-yl)amino)but-2-enamide

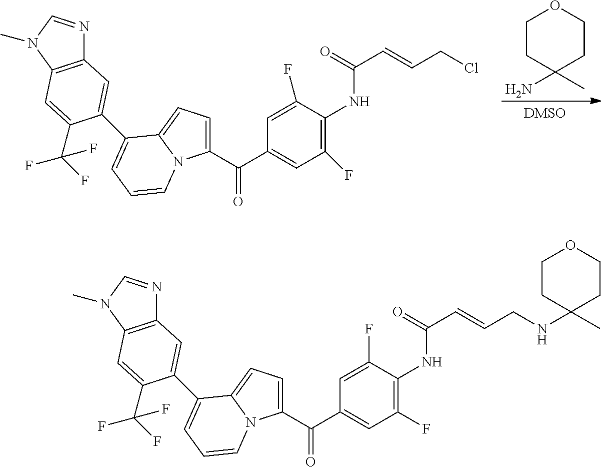

To a solution of the (E)-4-chloro-N-(2,6-difluoro-4-(8-(1-methyl-6-(trifluoromethyl)-1H-benzo[d]imidazol-5-yl)indolizine-3-carbonyl)phenyl)but-2-enamide (40 mg, 0.0698 mmol) in DMSO (0.6 mL) was added 4-methyltetrahydro-2H-pyran-4-amine (0.086 mL, 0.698 mmol). The mixture was stirred at 40° C. for 100 min. The resulting mixture was purified by preparative HPLC (water:acetonitrile (0.1% formic acid)) to give the title compound (29.7 mg, 65%).

### Example 12-1 and 12-2

### (E)-N-(4-(8-(4-chloro-1,6-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

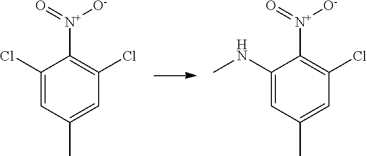

The mixture of 1,3-dichloro-5-methyl-2-nitrobenzene (37.0 g. 180 mmol) and 2M methylamine in THF (242 mL, 485 mmol) was stirred at 80° C. overnight. To the resulting mixture was added EtOAc and H₂O, and extracted with EtOAc, the organic layer was separated, and washed with brine, dried over sodium sulfate, and evaporated. The residue was purified by column chromatography on silica gel (gradient elution, hexane-EtOAc) to give 3-chloro-N,5-dimethyl-2-nitroaniline (11.2 g).

### Step 2

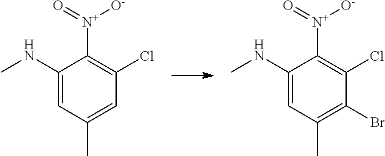

NBS (1.13 g, 6.36 mmol) was added to a solution of 3-chloro-N,5-dimethyl-2-nitroaniline (1.16 g. 5.78 mmol) in DMF (20 mL) at 0° C. and the mixture was stirred at RT for 20 min. To the resulting mixture was added EtOAc and H₂O, and extracted with EtOAc, the organic layer was separated, and washed with H₂O and brine, dried over sodium sulfate, and evaporated to give crude 4-bromo-3-chloro-N,5-dimethyl-2-nitroaniline (1.62 g).

### Step 3

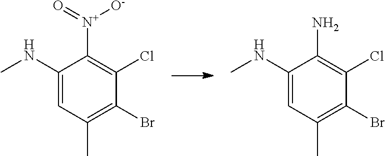

2M NH₄Cl aq. (5.8 mL, 11.6 mmol) was added to a solution of crude 4-bromo-3-chloro-N,5-dimethyl-2-nitroaniline (1.62 g) and Fe (1.61 g. 28.9 mmol) in THF (12 mL) and MeOH (12 mL) and the mixture was stirred at 70° C. for 30 min and 80° C. for 45 min. After cooling, the reaction was filtered through Celite, rinsing with EtOAc, and the filtrate was evaporated. To the residue was added EtOAc and H₂O, and extracted with EtOAc, the organic layer was separated, and washed with brine, dried over sodium sulfate, and evaporated to give crude 4-bromo-3-chloro-N1,5-dimethylbenzene-1,2-diamine (1.48 g).

### Step 4

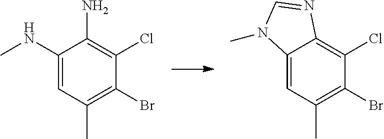

To crude 4-bromo-3-chloro-N1,5-dimethylbenzene-1,2-diamine (1.48 g) was added triethyl orthoformate (13.4 mL) and slowly added concentrated aqueous HCl (0.0482 mL). The mixture was stirred at RT for 10 min and neutralized by aqueous NaOH, then diluted with EtOAc and H₂O and extracted with EtOAc. The organic layer was washed with H₂O and brine, dried over sodium sulfate, and evaporated. The residue was suspended in IPE (10 mL) and the suspension was stirred at RT for 5 min. The precipitate was collected via filtration, washing with IPE, and the solid dried in vacuo to give 5-bromo-4-chloro-1,6-dimethyl-1H-benzo[d]imidazole (1.37 g).

### Step 5

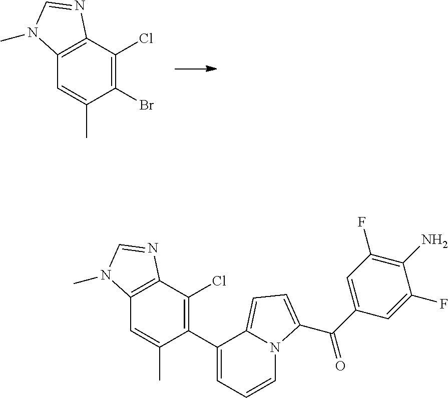 [SMILES: Cc1cc2c(ncn2C)c(Cl)c1Br ; Cc1cc2c(ncn2C)c(Cl)c1-c1cccn2c(C(=O)c3cc(F)c(N)c(F)c3)ccc12]

To a mixture of (4-amino-3,5-difluorophenyl)(8-(5,5-dimethyl-1,3,2-dioxaborinan-2-yl)indolizin-3-yl)methanone (207 mg,), 5-bromo-4-chloro-1,6-dimethyl-1H-benzo[d]imidazole (100 mg) and Pd(PPh3)4 (44.5 mg) in 1,2-dimethoxyethane (2.0 mL) was added 2M aqueous sodium carbonate solution (0.385 mL). After reacting by microwave apparatus at 120° C. for 3 hours, the mixture was diluted with EtOAc and water. The organic layer was washed with brine, dried over sodium sulfate and evaporated under reduced pressure. The residue was purified by column chromatography on silica gel (CHCl₃-methanol) to give (4-amino-3,5-difluorophenyl)(8-(4-chloro-1,6-dimethyl-1H-benzo[d]imidazol-5-yl)indolizin-3-yl)methanone (160 mg).

### Step 6

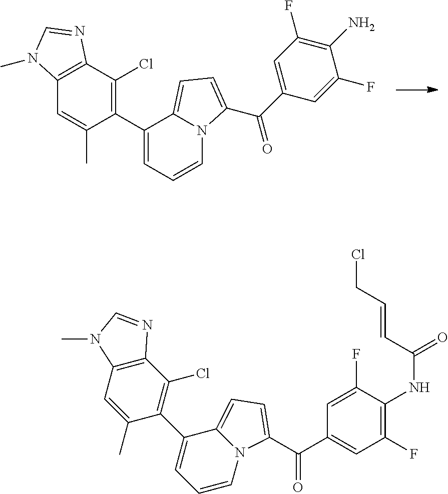 [SMILES: Cc1cc2c(ncn2C)c(Cl)c1-c1cccn2c(C(=O)c3cc(F)c(N)c(F)c3)ccc12 ; Cc1cc2c(ncn2C)c(Cl)c1-c1cccn2c(C(=O)c3cc(F)c(NC(=O)/C=C/CCl)c(F)c3)ccc12]

The procedure of Example 2 (step 2) was performed except that (4-amino-3,5-difluorophenyl)(8-(4-chloro-1,6-dimethyl-1H-benzo[d]imidazol-5-yl)indolizin-3-yl)methanone (160 mg) obtained in step 5 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2), thereby obtaining crude (E)-4-chloro-N-(4-(8-(4-chloro-1,6-dimethyl-1H-benzo[d]imidazol-5-yl)indolizine-3-carbonyl)-2,6-difluorophenyl)but-2-enamide (178 mg).

### Step 7

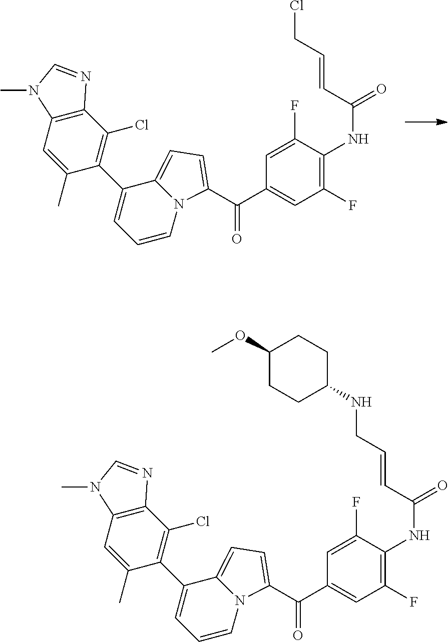 [SMILES: Cc1cc2c(ncn2C)c(Cl)c1-c1cccn2c(C(=O)c3cc(F)c(NC(=O)/C=C/CCl)c(F)c3)ccc12 ; CO[C@H]1CC[C@H](NC/C=C/C(=O)Nc2c(F)cc(C(=O)c3ccc4c(-c5c(C)cc6c(ncn6C)c5Cl)cccn34)cc2F)CC1]

The procedure of Example 2 (step 3) was performed except that crude (E)-4-chloro-N-(4-(8-(4-chloro-1,6-dimethyl-1H-benzo[d]imidazol-5-yl)indolizine-3-carbonyl)-2,6-difluorophenyl)but-2-enamide (89 mg) obtained in step 6 was used instead of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide in Example 2 (step 3), thereby obtaining the racemic title compound (47.5 mg). The racemate was separated by CHIRAL ART SB (hexane-ethanol (0.1% triethyl amine)) to give the title compound (Example 12-1, 16.3 mg; single isomer, 1^{st}-eluting isomer and Example 12-2, 15.9 mg; single isomer, 2^{nd}-eluting isomer).

### Example 13-1 and 13-2

### (E)-4-(tert-butylamino)-N-(4-(8-(4-chloro-1,6-dimethyl-1H-benzo[d]imidazol-5-yl)indolizine-3-carbonyl)-2,6-difluorophenyl)but-2-enamide

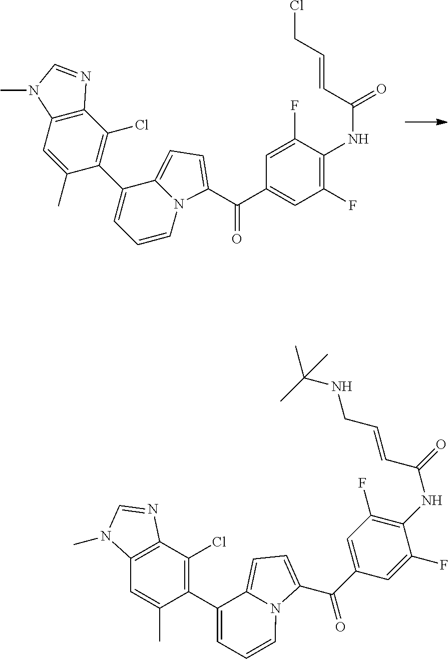 [SMILES: Cc1cc2c(ncn2C)c(Cl)c1-c1cccn2c(C(=O)c3cc(F)c(NC(=O)/C=C/CCl)c(F)c3)ccc12 ; Cc1cc2c(ncn2C)c(Cl)c1-c1cccn2c(C(=O)c3cc(F)c(NC(=O)/C=C/CNC(C)(C)C)c(F)c3)ccc12]

A solution of crude (E)-4-chloro-N-(4-(8-(4-chloro-1,6-dimethyl-1H-benzo[d]imidazol-5-yl)indolizine-3-carbonyl)-2,6-difluorophenyl)but-2-enamide (89 mg), KI (88.6 mg, 0.534 mmol) and tert-butylamine (0.187 mL, 1.78 mmol) in DMF (1.0 mL) was stirred at RT for 3.5 hours. To the resulting mixture was added H₂O and EtOAc. The organic layer was washed with H₂O and brine dried over sodium sulfate, and evaporated. The residue was purified by column chromatography on silica gel (CHCl₃-methanol), thereby obtaining the racemic title compound (48.5 mg). The racemate was separated by CHIRAL ART SB (hexane-ethanol (0.1% triethyl amine)) to give the title compound (Example 13-1, 15.2 mg; single isomer. 1^{st}-eluting isomer and Example 13-2, 15.7 mg; single isomer. 2^{nd}-eluting isomer).

### Example 14-1 and 14-2

### (E)-4-(tert-butylamino)-N-(4-(8-(4-chloro-6-methoxy-1-methyl-1H-benzo[d]imidazol-5-yl)indolizine-3-carbonyl)-2,6-difluorophenyl)but-2-enamide

### Step 1

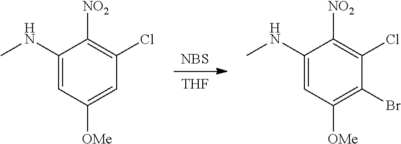

To a solution of 3-chloro-5-methoxy-N-methyl-2-nitroaniline (229 mg, 1.06 mmol) in THF (3 mL) was added N-Bromosuccinimide (208 mg, 1.17 mmol) at room temperature, and the mixture was stirred for 15 min. Saturated aqueous NaHCO₃ aq. Na₂S₂O₃ and AcOEt were added to the reaction mixture. The organic layer was separated, washed with brine, dried over Na₂SO₄, and evaporated. The residue was diluted with chloroform and diisopropyl ether and the precipitates were collected by filtration to give 4-bromo-3-chloro-5-methoxy-N-methyl-2-nitroaniline (253 mg, 81%).

### Step 2

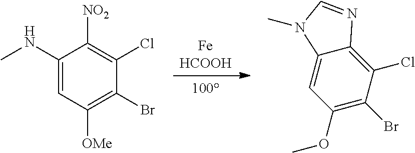

The mixture of 4-bromo-3-chloro-5-methoxy-N-methyl-2-nitroaniline (186 mg. 0.629 mmol) in formic acid (3 mL) was stirred at 100° C. for 10 min. Iron powder (272 mg, 4.87 mmol) was added thereto, and the mixture was stirred at 100° C. overnight. After cooling, the resulting mixture was diluted with EtOAc and saturated aqueous NaHCO₃, and the insolubles were removed by filtration. The organic layer was separated, washed with brine, dried over sodium sulfate, and evaporated. The residue was purified by column chromatography on silica gel (gradient elution, hexane-EtOAc) to give 5-bromo-4-chloro-6-methoxy-1-methyl-1H-benzo[d]imidazole (47 mg, 27%).

### Step 3

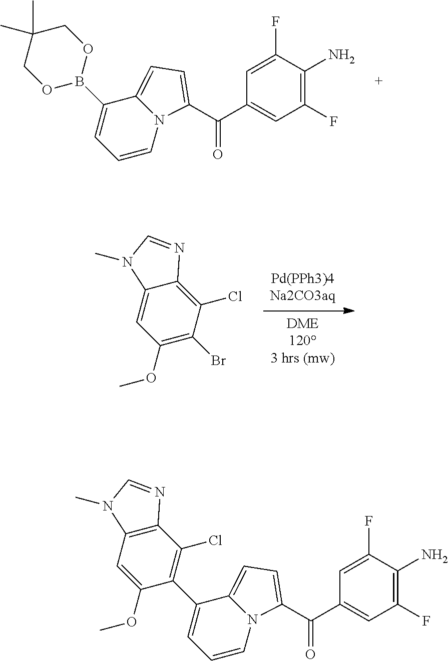 [SMILES: CC1(C)COB(c2cccn3c(C(=O)c4cc(F)c(N)c(F)c4)ccc23)OC1 ; COc1cc2c(ncn2C)c(Cl)c1Br ; "DME120°3 hrs (mw)" ; "Pd(PPh3)4Na2CO3aq" ; COc1cc2c(ncn2C)c(Cl)c1-c1cccn2c(C(=O)c3cc(F)c(N)c(F)c3)ccc12]

To a mixture of (4-amino-3,5-difluorophenyl)(8-(5,5-dimethyl-1,3,2-dioxaborinan-2-yl)indolizin-3-yl)methanone (88.7 mg, 0.231 mmol). 5-bromo-4-chloro-6-methoxy-1-methyl-1H-benzo[d]imidazole (53 mg, 0.192 mmol) and Pd(PPh₃)₄ (22.2 mg. 0.019 mmol) in 1,2-dimethoxyethane (1.5 mL) was added 2M aqueous sodium carbonate solution (0.192 mL, 0.385 mmol). After reacting by microwave apparatus at 120° C. for 3 hours, the mixture was diluted with EtOAc and water. The organic layer was washed with brine, dried over sodium sulfate and evaporated under reduced pressure. The residue was purified by column chromatography on silica gel (CHCl₃-EtOH) to give the crude of (4-amino-3,5-difluorophenyl)(8-(4-chloro-6-methoxy-1-methyl-1H-benzo[d]imidazol-5-yl)indolizin-3-yl)methanone (98 mg).

### Step 4

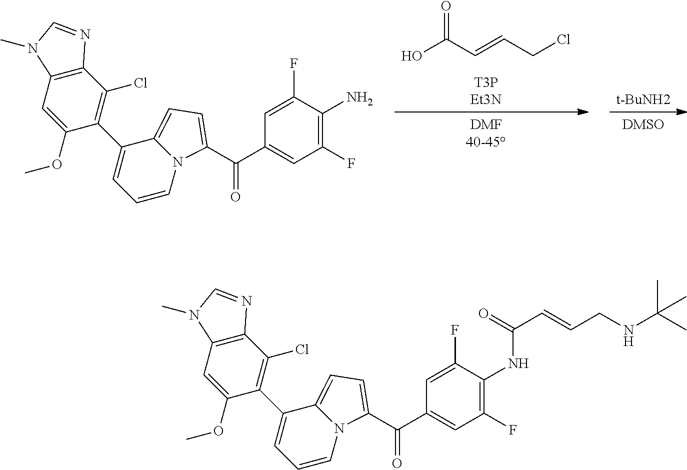

The mixture of the above (4-amino-3,5-difluorophenyl)(8-(4-chloro-6-methoxy-1-methyl-1H-benzo[d]imidazol-5-yl)indolizin-3-yl)methanone (89 mg, 191 mmol), 4-chlorocrotonic acid (68.9 mg, 0.572 mmol) and propylphosphonic anhydride solution (50 wt % in THF. 0.596 mL, 0.953 mmol) in DMF (2 mL) was heated at 40° C. for 15 min. Then triethylamine (0.159 mL, 1.14 mmol) was added thereto, and the mixture was stirred at room temperature for 30 min. To the resulting mixture was added saturated aqueous NaHCO₃, and extracted with EtOAc. and washed successively with H₂O then brine, dried over sodium sulfate, and evaporated. To the residue was added DMSO (2 mL) and tert-butylamine (0.238 mL). The mixture was stirred at room temperature overnight. The resulting mixture was purified by preparative HPLC (water:acetonitrile (0.1% formic acid)) to give the racemic title compound (55.5 mg, 48%). The racemate was separated by CHIRALPAK ID (hexane-ethanol(0.1/triethyl amine)) to give the title compound (Example 14-1, 23.3 mg: single isomer, 1^{st}-eluting isomer and Example 14-2, 24.4 mg: single isomer, 2^{nd}-eluting isomer).

### Example 15-1 and 15-2

### (E)-N-(4-(8-(4,6-dichloro-1-methyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

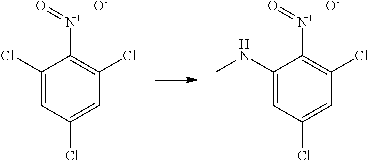

The mixture of 1,3,5-trichloro-2-nitrobenzene (3.00 g, 13.2 mmol) and 2M methylamine in THF (33.0 mL, 66.2 mmol) was stirred at 30° C. for 17 hours and at RT for 2 days. To the resulting mixture was added EtOAc and H₂O, the organic layer was separated, and washed with brine, dried over sodium sulfate, and evaporated. The residue was suspended in IPE (30 mL) and the suspension was stirred at RT for 15 min. The suspension was filtered, rinsing with IPE, and the filtrate was evaporated. The residue was purified by column chromatography on silica gel (gradient elution, hexane-EtOAc) to give 3,5-dichloro-N-methyl-2-nitroaniline 1.05 g).

### Step 2

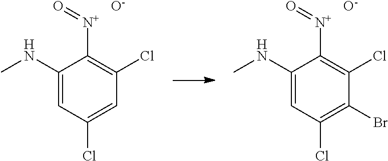

NBS (930 mg, 5.23 mmol) was added to a solution of 3,5-dichloro-N-methyl-2-nitroaniline (1.05 g. 4.75 mmol) in DMF (20 mL) at RT and the mixture was stirred at RT for 25 min. To the resulting mixture was added EtOAc and H₂O, and extracted with EtOAc, the organic layer was separated, and washed with H₂O and brine, dried over sodium sulfate, and evaporated to give crude 4-bromo-3,5-dichloro-N-methyl-2-nitroaniline (1.63 g).

### Step 3

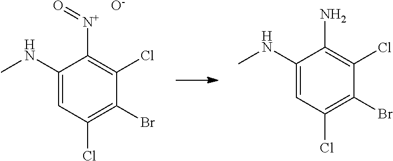

2M NH₄Cl aq. (5.0 mL, 10 mmol) was added to a solution of crude 4-bromo-3,5-dichloro-N-methyl-2-nitroaniline (1.63 g) and Fe (1.33 g, 23.8 mmol) in THF (10 mL) and MeOH (10 mL) and the mixture was stirred at 70° C. for 3 hours. After cooling, the reaction was filtered through Celite, rinsing with EtOAc, and the filtrate was evaporated. To the residue was added EtOAc and H₂O, and the organic layer was separated, and washed with brine, dried over sodium sulfate, and evaporated to give crude 4-bromo-3,5-dichloro-N1-methylbenzene-1,2-diamine (1.32 g).

### Step 4

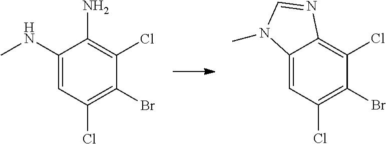

To crude 4-bromo-3,5-dichloro-N1-methylbenzene-1,2-diamine (1.32 g) was added triethyl orthoformate (12.9 mL) and slowly added concentrated aqueous HCl (0.0515 mL). The mixture was stirred at RT for 15 min and neutralized by aqueous NaOH, then diluted with EtOAc and H₂O and extracted with EtOAc. The organic layer was washed with brine, dried over sodium sulfate, and evaporated. The residue was suspended in EtOAc/Heptane=6/1 (10 mL) and the suspension was stirred at RT for 55 min. The precipitate was collected via filtration, washing with EtOAc/Heptane=6/1, and the solid dried in vacuo to give 5-bromo-4,6-dichloro-1-methyl-1H-benzo[d]imidazole (799 mg). The filtrate was evaporated and the residue was suspended in IPE (5 mL) and the suspension was stirred at RT for 25 min. The precipitate was collected via filtration, washing with IPE, and the solid dried in vacuo to give 5-bromo-4,6-dichloro-1-methyl-1H-benzo[d]imidazole (446 mg).

### Step 5

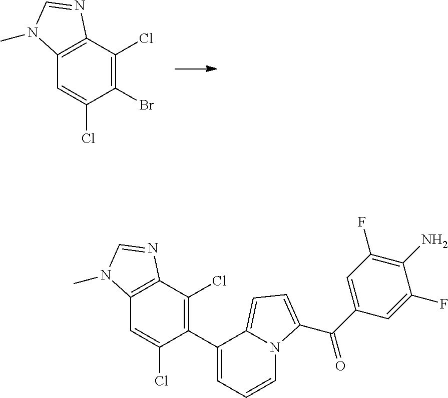 [SMILES: Cn1cnc2c(Cl)c(Br)c(Cl)cc21 ; Cn1cnc2c(Cl)c(-c3cccn4c(C(=O)c5cc(F)c(N)c(F)c5)ccc34)c(Cl)cc21]

To a mixture of (4-amino-3,5-difluorophenyl)(8-(5,5-dimethyl-1,3,2-dioxaborinan-2-yl)indolizin-3-yl)methanone (178 mg,), 5-bromo-4,6-dichloro-1-methyl-1H-benzo[d]imidazole (100 mg) and Pd(PPh₃)₄ (41.3 mg) in 1,2-dimethoxyethane (2.0 mL) was added 2M aqueous sodium carbonate solution (0.357 mL). After reacting by microwave apparatus at 120° C. for 3 hours, the mixture was diluted with EtOAc and water. The organic layer was washed with brine, dried over sodium sulfate and evaporated under reduced pressure. The residue was purified by column chromatography on silica gel (CHCl₃-methanol) to give crude (4-amino-3,5-difluorophenyl)(8-(4,6-dichloro-1-methyl-1H-benzo[d]imidazol-5-yl)indolizin-3-yl)methanone (176 mg).

### Step 6

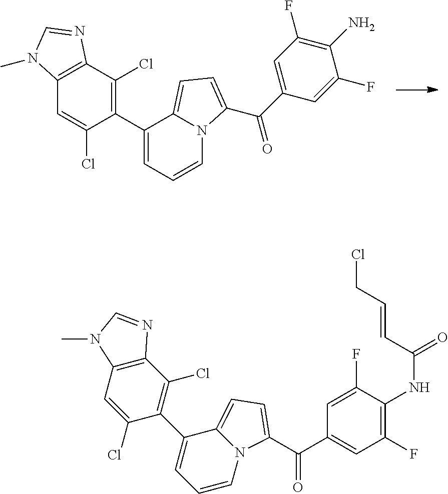 [SMILES: Cn1cnc2c(Cl)c(-c3cccn4c(C(=O)c5cc(F)c(N)c(F)c5)ccc34)c(Cl)cc21 ; Cn1cnc2c(Cl)c(-c3cccn4c(C(=O)c5cc(F)c(NC(=O)/C=C/CCl)c(F)c5)ccc34)c(Cl)cc21]

The procedure of Example 2 (step 2) was performed except that crude (4-amino-3,5-difluorophenyl)(8-(4,6-dichloro-1-methyl-1H-benzo[d]imidazol-5-yl)indolizin-3-yl)methanone (176 mg) obtained in step 5 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2), thereby obtaining crude (E)-4-chloro-N-(4-(8-(4,6-dichloro-1-methy-1H-benzo[d]imidazol-5-yl)indolizine-3-carbonyl)-2,6-difluorophenyl)but-2-enamide (125 mg).

### Step 7

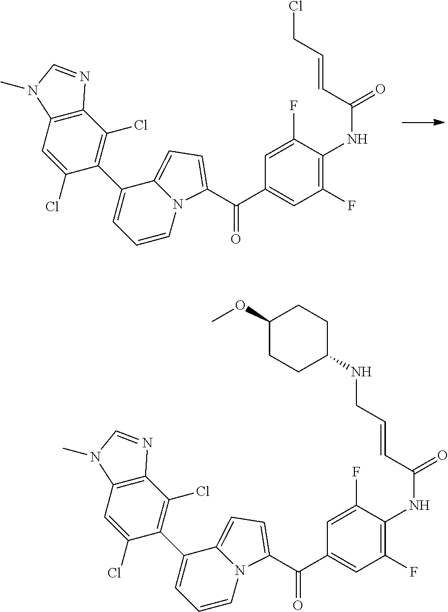 [SMILES: Cn1cnc2c(Cl)c(-c3cccn4c(C(=O)c5cc(F)c(NC(=O)/C=C/CCl)c(F)c5)ccc34)c(Cl)cc21 ; CO[C@H]1CC[C@H](NC/C=C/C(=O)Nc2c(F)cc(C(=O)c3ccc4c(-c5c(Cl)cc6c(ncn6C)c5Cl)cccn34)cc2F)CC1]

The procedure of Example 2 (step 3) was performed except that crude (E)-4-chloro-N-(4-(8-(4,6-dichloro-1-methyl-1H-benzo[d]imidazol-5-yl)indolizine-3-carbonyl)-2,6-difluorophenyl)but-2-enamide (61.5 mg) obtained in step 6 was used instead of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide in Example 2 (step 3), thereby obtaining the racemic title compound (27.1 mg). The racemate was separated by CHIRAL ART SB (hexane-ethanol (0.1% triethyl amine)) to give the title compound (Example 15-1, 8.77 mg: single isomer, 1^{st}-eluting isomer and Example 15-2, 8.99 mg: single isomer, 2^{nd}-eluting isomer).

### Example 16

(E)-4-(tert-butylamino)-N-(4-(8-(4-chloro-6-ethyl-1,2-dimethyl-1H-benzo[d]imidazol-5-yl)indolizine-3-carbonyl)-2,6-difluorophenyl)but-2-enamide

### Step 1

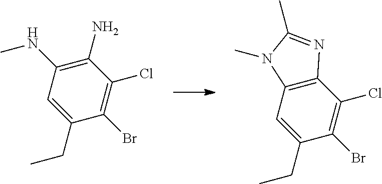

The procedure of Example 10 (step 5) was performed except that 1,1,1-triethoxyethane was used instead of triethoxymethane, thereby obtaining 5-bromo-4-chloro-6-ethyl-1,2-dimethyl-1H-benzo[d]imidazole.

### Step 2

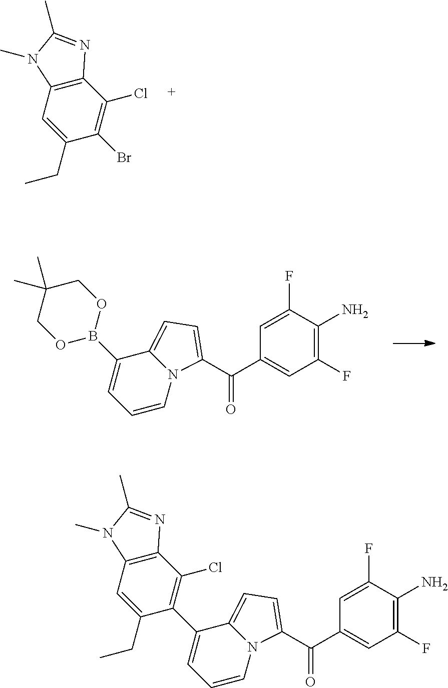 [SMILES: CCc1cc2c(nc(C)n2C)c(Cl)c1Br ; CC1(C)COB(c2cccn3c(C(=O)c4cc(F)c(N)c(F)c4)ccc23)OC1 ; CCc1cc2c(nc(C)n2C)c(Cl)c1-c1cccn2c(C(=O)c3cc(F)c(N)c(F)c3)ccc12]

The procedure of Example 10 (step 6) was performed except that 5-bromo-4-chloro-6-ethyl-1,2-dimethyl-1H-benzo[d]imidazole (37.4 mg) obtained in step 1 was used instead of 5-bromo-4-chloro-6-ethyl-1-methyl-1H-benzo[d]imidazole used in Example 10 (step 6), thereby obtaining (4-amino-3,5-difluorophenyl)(8-(4-chloro-6-ethyl-1,2-dimethyl-1H-benzo[d]imidazol-5-yl)indolizin-3-yl)methanone (26.0 mg).

### Step 3

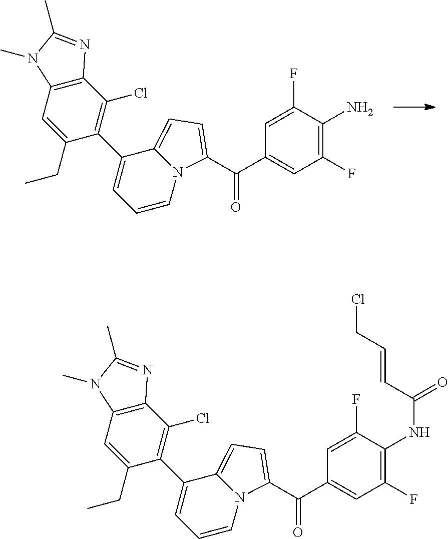 [SMILES: CCc1cc2c(nc(C)n2C)c(Cl)c1-c1cccn2c(C(=O)c3cc(F)c(N)c(F)c3)ccc12 ; CCc1cc2c(nc(C)n2C)c(Cl)c1-c1cccn2c(C(=O)c3cc(F)c(NC(=O)/C=C/CCl)c(F)c3)ccc12]

The procedure of Example 10 (step 7) was performed except that (4-amino-3,5-difluorophenyl)(8-(4-chloro-6-ethyl-1,2-dimethyl-1H-benzo[d]imidazol-5-yl)indolizin-3-yl)methanone (26.0 mg) obtained in step 2 was used instead of (4-amino-3,5-difluorophenyl)(8-(4-chloro-6-ethyl-1-methyl-1H-benzo[d]imidazol-5-yl)indolizin-3-yl)methanone used in Example 10 (step 7), thereby obtaining (E)-4-chloro-N-(4-(8-(4-chloro-6-ethyl-1,2-dimethyl-1H-benzo[d]imidazol-5-yl)indolizine-3-carbonyl)-2,6-difluorophenyl)but-2-enamide (31.0 mg).

### Step 4

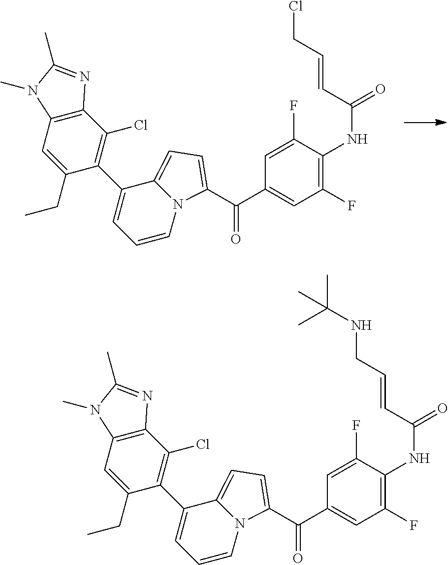 [SMILES: CCc1cc2c(nc(C)n2C)c(Cl)c1-c1cccn2c(C(=O)c3cc(F)c(NC(=O)/C=C/CCl)c(F)c3)ccc12 ; CCc1cc2c(nc(C)n2C)c(Cl)c1-c1cccn2c(C(=O)c3cc(F)c(NC(=O)/C=C/CNC(C)(C)C)c(F)c3)ccc12]

The procedure of Example 10 (step 8) was performed except that (E)-4-chloro-N-(448-(4-chloro-6-ethyl-1,2-dimethyl-1H-benzo[d]imidazol-5-yl)indolizine-3-carbonyl)-2,6-difluorophenyl)but-2-enamide (31.0 mg) obtained in step 3 and 2-methylpropan-2-amine were used instead of (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide used in Example 10 (step 8) and (1r,4r)-4-methoxycyclohexan-1-amine hydrochloride, thereby obtaining the title compound (10.6 mg).

### Example 17

(E)-N-(4-(8(4-chloro-6-ethyl-1,2-dimethyl-1H-benzo[d]imidazol-5-yl)indolizine-3-carbonyl)-2,6-difluorophenyl)-4-((tetrahydro-2H-pyran-4-yl)amino)but-2-enamide

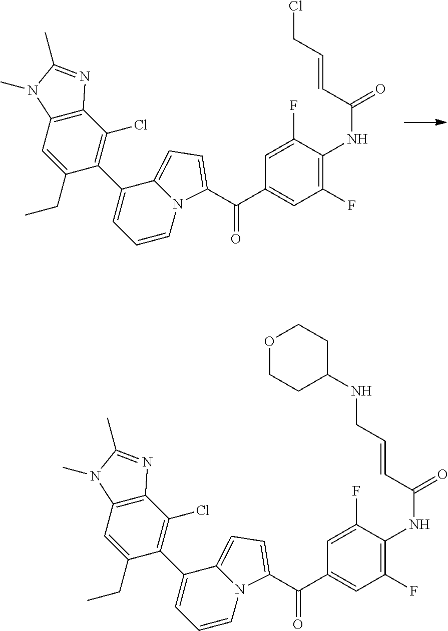 [SMILES: CCc1cc2c(nc(C)n2C)c(Cl)c1-c1cccn2c(C(=O)c3cc(F)c(NC(=O)/C=C/CCl)c(F)c3)ccc12 ; CCc1cc2c(nc(C)n2C)c(Cl)c1-c1cccn2c(C(=O)c3cc(F)c(NC(=O)/C=C/CNC4CCOCC4)c(F)c3)ccc12]

The procedure of Example 16 (step 4) was performed except that tetrahydro-2H-pyran-4-amine was used instead of 2-methylpropan-2-amine, thereby obtaining the title compound (8.98 mg).

### Example 18-1 and 18-2

### (E)-4-(tert-butylamino)-N-(4-(8-(4-chloro-1,2,6-trimethyl-1H-benzo[d]imidazol-5-yl)indolizine-3-carbonyl)-2,6-difluorophenyl)but-2-enamide

### Step 1

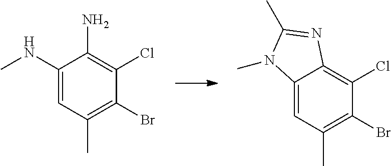

To 4-bromo-3-chloro-N1,5-dimethylbenzene-1,2-diamine (2.00 g) was added trimethyl orthoacetate (18.0 mL) and slowly added concentrated aqueous HCl (0.668 mL). The mixture was stirred at RT for 85 min and trimethyl orthoacetate (2.0 mL) was added and the mixture was stirred at RT for 2 hours and at 40° C. for 2 hours. AcOH (0.459 mL) was added at RT and the mixture was stirred at 50° C. for 2 hours and AcOH (0.459 mL) was added at RT and the mixture was stirred at 50° C. for 1 hour and at RT overnight. The mixture was neutralized by aqueous NaOH, then diluted with H₂O and the suspension was stirred at RT for 100 min. The precipitate was collected via filtration, washing with H₂O and Heptane, and the solid dried in vacuo to give 5-bromo-4-chloro-1,2,6-trimethyl-1H-benzo[d]imidazole (2.07 g).

### Step 2

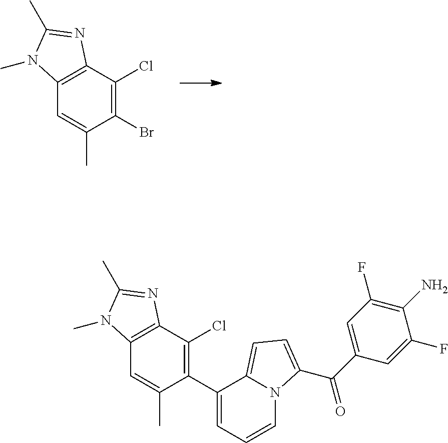 [SMILES: Cc1cc2c(nc(C)n2C)c(Cl)c1Br ; Cc1cc2c(nc(C)n2C)c(Cl)c1-c1cccn2c(C(=O)c3cc(F)c(N)c(F)c3)ccc12]

To a mixture of (4-amino-3,5-difluorophenyl)(8-(5,5-dimethyl-1,3,2-dioxaborinan-2-yl)indolizin-3-yl)methanone (183 mg). 5-bromo-4-chloro-1,2,6-trimethyl-1H-benzo[d]imidazole (100 mg) and Pd(PPh3)4 (42.2 mg) in 1,2-dimethoxyethane (3.0 mL) was added 2M aqueous sodium carbonate solution (0.366 mL). After reacting by microwave apparatus at 120° C. for 3 hours, the mixture was diluted with EtOAc, water, and brine. The organic layer was washed with brine, dried over sodium sulfate and evaporated under reduced pressure. The residue was purified by column chromatography on silica gel (CHCl₃-methanol) to give crude (4-amino-3,5-difluorophenyl)(8-(4-chloro-1,2,6-trimethyl-1H-benzo[d]imidazol-5-yl)indolizin-3-yl)methanone (214 mg).

### Step 3

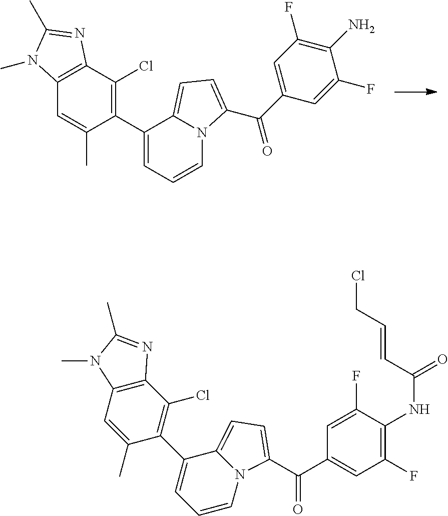 [SMILES: Cc1cc2c(nc(C)n2C)c(Cl)c1-c1cccn2c(C(=O)c3cc(F)c(N)c(F)c3)ccc12 ; Cc1cc2c(nc(C)n2C)c(Cl)c1-c1cccn2c(C(=O)c3cc(F)c(NC(=O)/C=C/CCl)c(F)c3)ccc12]

The procedure of Example 2 (step 2) was performed except that crude (4-amino-3,5-difluorophenyl)(8-(4-chloro-1,2,6-trimethyl-1H-benzo[d]imidazol-5-yl)indolizin-3-yl)methanone (214 mg) obtained in step 2 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2), thereby obtaining crude (E)-4-chloro-N-(4-(8-(4-chloro-1,2,6-trimethyl-1H-benzo[d]imidazol-5-yl)indolizine-3-carbonyl)-2,6-difluorophenyl)but-2-enamide (204 mg).

### Step 4

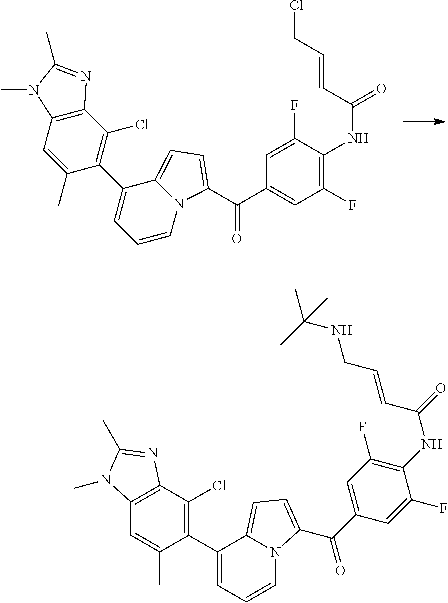 [SMILES: Cc1cc2c(nc(C)n2C)c(Cl)c1-c1cccn2c(C(=O)c3cc(F)c(NC(=O)/C=C/CCl)c(F)c3)ccc12 ; Cc1cc2c(nc(C)n2C)c(Cl)c1-c1cccn2c(C(=O)c3cc(F)c(NC(=O)/C=C/CNC(C)(C)C)c(F)c3)ccc12]

A solution of crude (E)-4-chloro-N-(4-(8-(4-chloro-1,2,6-trimethyl-1H-benzo[d]imidazol-5-yl)indolizine-3-carbonyl)-2,6-difluorophenyl)but-2-enamide (102 mg). KI (89.6 mg, 0.540 mmol) and tert-butylamine (0.189 mL, 1.80 mmol) in DMF (1.0 mL) was stirred at RT for 2 hours. To the resulting mixture was added H₂O and EtOAc. The organic layer was washed with H₂O and brine, then dried over sodium sulfate, and evaporated. The residue was purified by column chromatography on silica gel (CHCl₃-methanol), thereby obtaining the racemic title compound (51.0 mg). The racemate was separated by CHIRAL ART SB (hexane-ethanol (0.1% triethyl amine)) to give the title compound (Example 18-1, 17.0 mg; single isomer, 1^{st}-eluting isomer and Example 18-2, 17.3 mg; single isomer, 2^{nd}-eluting isomer).

### Example 19-1 and 19-2

### (E)-N-(4-(8-(4-chloro-1,2,6-trimethyl-1H-benzo[d]imidazol-5-yl)indolizine-3-carbonyl)-2,6-difluorophenyl)-4-(((1r,4r)-4-methoxycyclohexyl)amino)but-2-enamide

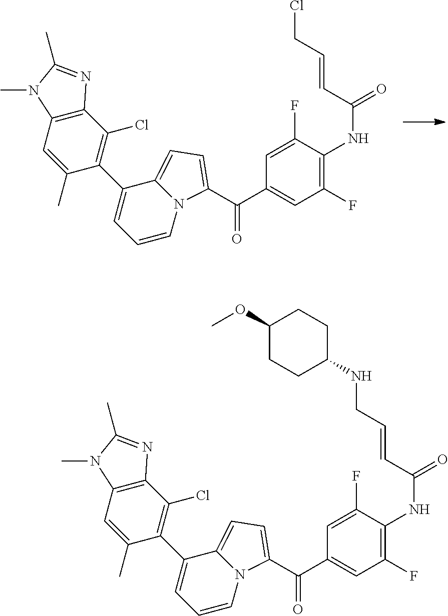 [SMILES: Cc1cc2c(nc(C)n2C)c(Cl)c1-c1cccn2c(C(=O)c3cc(F)c(NC(=O)/C=C/CCl)c(F)c3)ccc12 ; CO[C@H]1CC[C@H](NC/C=C/C(=O)Nc2c(F)cc(C(=O)c3ccc4c(-c5c(C)cc6c(nc(C)n6C)c5Cl)cccn34)cc2F)CC1]

The procedure of Example 2 (step 3) was performed except that crude (E)-4-chloro-N-(4-(8-(4-chloro-1,2,6-trimethyl-1H-benzo[d]imidazol-5-yl)indolizine-3-carbonyl)-2,6-difluorophenyl)but-2-enamide (102 mg) obtained in Example 18 (step 3) was used instead of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide in Example 2 (step 3), thereby obtaining the racemic title compound (44.9 mg). The racemate was separated by CHIRAL ART SB (hexane-ethanol (0.1% triethyl amine)) to give the title compound (Example 19-1, 14.1 mg; single isomer, 1^{st}-eluting isomer and Example 19-2, 14.0 mg; single isomer, 2^{nd}-eluting isomer).

### Example 20-1 and 20-2

### (E)-4-(tert-butylamino)-N-(4-(8-(4,6-dichloro-1-methyl-1H-benzo[d]imidazol-5-yl)indolizine-3-carbonyl)-2,6-difluorophenyl)but-2-enamide

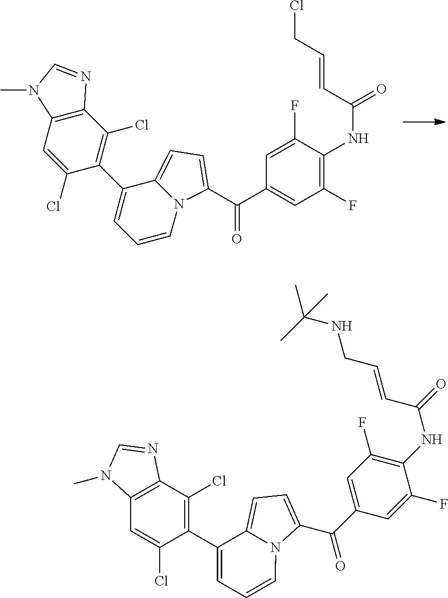 [SMILES: Cn1cnc2c(Cl)c(-c3cccn4c(C(=O)c5cc(F)c(NC(=O)/C=C/CCl)c(F)c5)ccc34)c(Cl)cc21 ; Cn1cnc2c(Cl)c(-c3cccn4c(C(=O)c5cc(F)c(NC(=O)/C=C/CNC(C)(C)C)c(F)c5)ccc34)c(Cl)cc21]

A solution of crude crude (E)-4-chloro-N-(4-(8-(4,6-dichloro-1-methyl-1H-benzo[d]imidazol-5-yl)indolizine-3-carbonyl)-2,6-difluorophenyl)but-2-enamide (61.5 mg), KI (53.4 mg, 0.322 mmol) and tert-butylamine (0.113 mL, 1.07 mmol) in DMF (1.5 mL) was stirred at RT for 5 hours. To the resulting mixture was added H₂O and EtOAc. The organic layer was washed with H₂O and brine, then dried over sodium sulfate, and evaporated. The residue was purified by preparative HPLC (water:acetonitrile (0.1% formic acid)). Saturated sodium bicarbonate water and H₂O were added to the purified fractions containing the racemic title compound, followed by extraction with ethyl acetate. The extract was washed with a saturated sodium chloride solution and dried over sodium sulfate. The solvent was evaporated under reduced pressure, thereby obtaining the racemic title compound (34.1 mg). The racemate was separated by CHIRALPAK ID (bexane-ethanol (0.1% triethyl amine)) to give the title compound (Example 20-1, 13.0 mg; single isomer, 1^{st}-eluting isomer and Example 20-2, 13.1 mg; single isomer, 2^{nd}-eluting isomer).

### Example 21

### (E)-4-(tert-butylamino)-N-(4-(8-(2-chloro-6-methoxy-4-(2-oxopyrrolidin-1-yl)phenyl)indolizine-3-carbonyl)-2,6-difluorophenyl)but-2-enamide

### Step 1

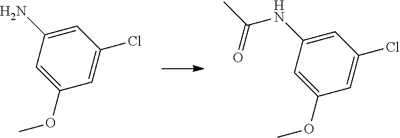

A solution of 3-chloro-5-methoxyaniline (500 mg), sodium acetate (781 mg) and acetic anhydride (486 mg) in dichloromethane (10.0 mL) was stirred at room temperature for 1 hour. After dilution with chloroform and water, the mixture was extracted with chloroform. The combined organic layer was washed with a saturated ammonium chloride solution and a saturated sodium chloride solution, followed by drying over sodium sulfate. The solvent was evaporated under reduced pressure, thereby obtaining crude N-(3-chloro-5-methoxyphenyl)acetamide (620 mg).

### Step 2

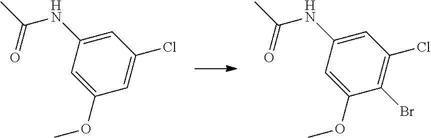

A solution of crude N-(3-chloro-5-methoxyphenyl)acetamide (620 mg) obtained in step 1 and N-bromosuccinimide (563 mg) in DMF (7.0 mL) was stirred at room temperature for 30 minutes. After dilution with ethyl acetate, the mixture was washed with water and a saturated sodium chloride solution, followed by drying over sodium sulfate. After evaporation, the residue was purified by column chromatography (hexane:ethyl acetate), thereby obtaining N-(4-bromo-3-chloro-5-methoxyphenyl)acetamide (540 mg).

### Step 3

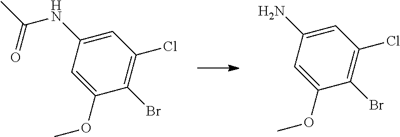

A solution of 5 M sodium hydroxide solution (2.0 mL) was added to a solution of N-(4-bromo-3-chloro-5-methoxyphenyl)acetamide (540 mg) obtained in step 2 in methanol (5.0 mL) and tetrahydrofuran (5.0 mL), followed by stirring at 70° C. for 14 hours. After cooling to room temperature, the reaction mixture was quenched with 5 M hydrochloric acid (2.0 mL). After dilution with ethyl acetate, the mixture was extracted with ethyl acetate. The combined organic layer was washed with water and a saturated sodium chloride solution, followed by drying over sodium sulfate. After evaporation, the residue was purified by column chromatography (hexane:ethyl acetate), thereby obtaining 4-bromo-3-chloro-5-methoxyaniline (480 mg).

### Step 4

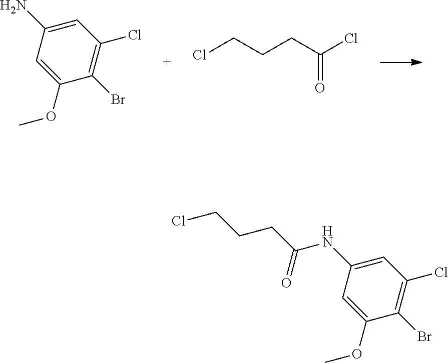 [SMILES: COc1cc(N)cc(Cl)c1Br ; O=C(Cl)CCCCl ; COc1cc(NC(=O)CCCCl)cc(Cl)c1Br]

4-chlorobutanoyl 1 chloride (272 μL) was added to a solution of 4-bromo-3-chloro-5-methoxyaniline (480 mg) obtained in step 3 and triethylamine (566 μL) in tetrahydrofuran (5.0 mL), followed by stirring at 0° C. for 1 hour. After dilution with ethyl acetate and water, the mixture was extracted with ethyl acetate. The combined organic layer was washed with water and a saturated sodium chloride solution, followed by drying over sodium sulfate. The solvent was evaporated under reduced pressure, thereby obtaining crude N-(4-bromo-3-chloro-5-methoxyphenyl)-4-chlorobutanamide (600 mg).

### Step 5

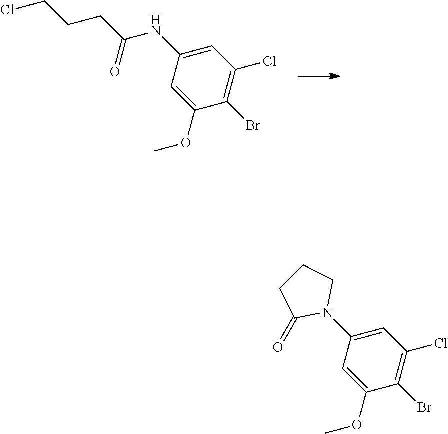 [SMILES: COc1cc(NC(=O)CCCCl)cc(Cl)c1Br ; COc1cc(N2CCCC2=O)cc(Cl)c1Br]

Sodium hydride (70 mg) was added to a solution of crude N-(4-bromo-3-chloro-5-methoxyphenyl)-4-chlorobutanamide (500 mg) obtained in step 4 in DMF (5.0 mL), followed by stirring at 60° C. for 1 hour. After dilution with ethyl acetate, the mixture was washed with water and a saturated sodium chloride solution, followed by drying over sodium sulfate. After evaporation, the residue was purified by column chromatography (hexane:ethyl acetate), thereby obtaining 1-(4-bromo-3-chloro-5-methoxyphenyl)pyrrolidin-2-one (414 mg).

### Step 6

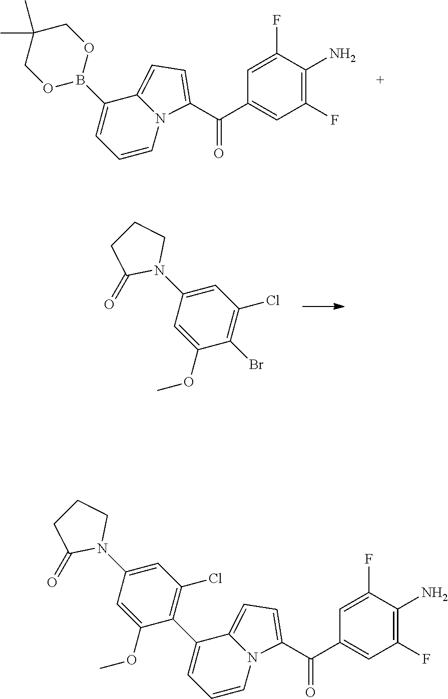 [SMILES: CC1(C)COB(c2cccn3c(C(=O)c4cc(F)c(N)c(F)c4)ccc23)OC1 ; COc1cc(N2CCCC2=O)cc(Cl)c1Br ; COc1cc(N2CCCC2=O)cc(Cl)c1-c1cccn2c(C(=O)c3cc(F)c(N)c(F)c3)ccc12]

A solution of 1-(4-bromo-3-chloro-5-methoxyphenyl)pyrrolidin-2-one (98.0 mg) obtained in step 5, (4-amino-3,5-difluorophenyl)(8-(5,5-dimethyl-1,3,2-dioxaborinan-2-yl)indolizin-3-yl)methanone (70.0 mg), tetrakis(triphenylphosphine)palladium(0) (29.5 mg), 2.0 M sodium bicarbonate in water (255 μL) in 1,2-dimethoxyethane (1.2 mL) was stirred at 120° C. for 2 hours using microwave irradiation. After dilution with ethyl acetate, the mixture was washed with water and a saturated sodium chloride solution, followed by drying over sodium sulfate. After evaporation, the residue was purified by column chromatography (hexane:ethyl acetate), thereby obtaining 1-(4-(3-(4-amino-3,5-difluorobenzoyl)indolizin-8-yl)-3-chloro-5-methoxyphenyl)pyrrolidin-2-one (95.0 mg).

### Step 7

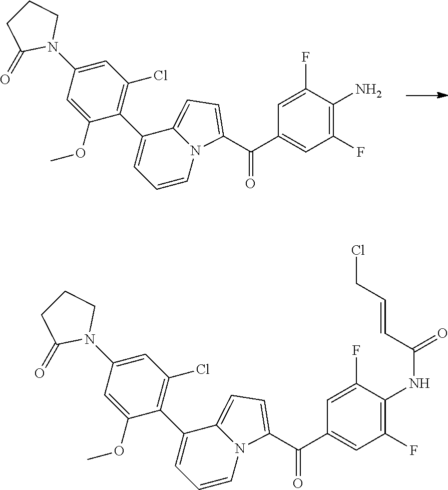 [SMILES: COc1cc(N2CCCC2=O)cc(Cl)c1-c1cccn2c(C(=O)c3cc(F)c(N)c(F)c3)ccc12 ; COc1cc(N2CCCC2=O)cc(Cl)c1-c1cccn2c(C(=O)c3cc(F)c(NC(=O)/C=C/CCl)c(F)c3)ccc12]

The procedure of Example 10 (step 7) was performed except that 1-(4-(3-(4-amino-3,5-difluorobenzoyl)indolizin-8-yl)-3-chloro-5-methoxyphenyl)pyrrolidin-2-one (95.0 mg) obtained in step 6 was used instead of (4-amino-3,5-difluorophenyl)(8-(4-chloro-6-ethyl-1-methyl-1H-benzo[d]imidazol-5-yl)indolizin-3-yl)methanone used in Example 10 (step 7), thereby obtaining (E)-4-chloro-N-(4-(8-(2-chloro-6-methoxy-4-(2-oxopyrrolidin-1-yl)phenyl)indolizine-3-carbonyl)-2,6-difluorophenyl)but-2-enamide (80.0 mg).

### Step 8

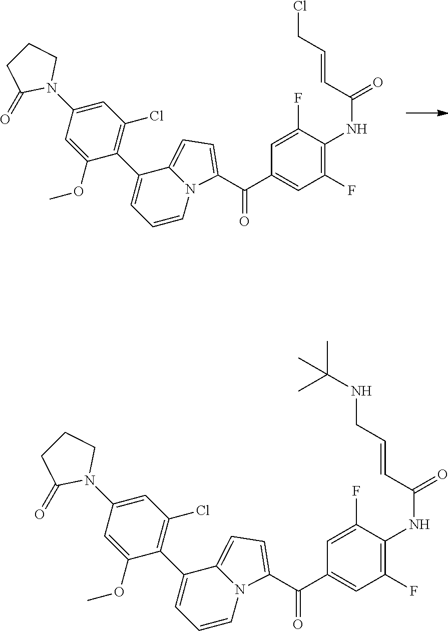 [SMILES: COc1cc(N2CCCC2=O)cc(Cl)c1-c1cccn2c(C(=O)c3cc(F)c(NC(=O)/C=C/CCl)c(F)c3)ccc12 ; COc1cc(N2CCCC2=O)cc(Cl)c1-c1cccn2c(C(=O)c3cc(F)c(NC(=O)/C=C/CNC(C)(C)C)c(F)c3)ccc12]

The procedure of Example 10 (step 8) was performed except that (E)-4-chloro-N-(4-(8-(2-chloro-6-methoxy-4-(2-oxopyrrolidin-1-yl)phenyl)indolizine-3-carbonyl)-2,6-difluorophenyl)but-2-enamide (20.0 mg) obtained in step 7 and 2-methylpropan-2-amine were used instead of (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide used in Example 10 (step 8) and (1r,4r)-4-methoxycyclohexan-1-amine hydrochloride, thereby obtaining the title compound (7.50 mg).

### Example 22-1 and 22-2

### (E)-N-(4-(8-(4-chloro-6-ethyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

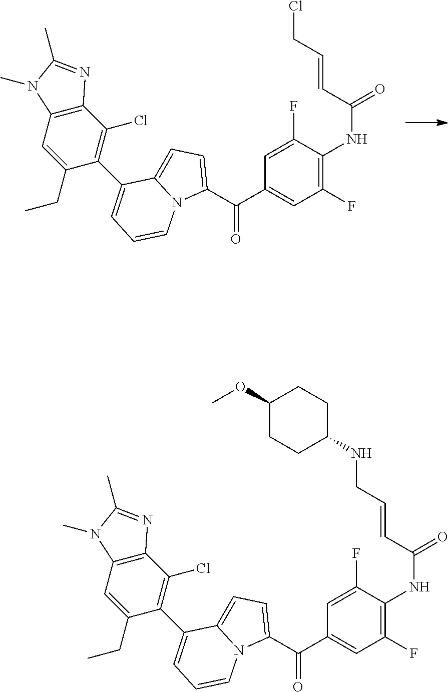 [SMILES: CCc1cc2c(nc(C)n2C)c(Cl)c1-c1cccn2c(C(=O)c3cc(F)c(NC(=O)/C=C/CCl)c(F)c3)ccc12 ; CCc1cc2c(nc(C)n2C)c(Cl)c1-c1cccn2c(C(=O)c3cc(F)c(NC(=O)/C=C/CN[C@H]4CC[C@H](OC)CC4)c(F)c3)ccc12]

The procedure of Example 10 (step 8) was performed except that (E)-4-chloro-N-(4-(8-(4-chloro-6-ethyl-1,2-dimethyl-1H-benzo[d]imidazol-5-yl)indolizine-3-carbonyl)-2,6-difluorophenyl)but-2-enamide (31.0 mg) obtained in step 3 (Example 16) was used instead of (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide used in Example 10 (step 8), thereby obtaining the title compound (15.4 mg).

### Step 2

(E)-N-(4-(8-(4-chloro-6-ethyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide (15.4 mg) obtained in step 1 was dissolved in EtOH and purified by preparative HPLC (CHIRAL ART SB, hexane-ethanol (0.1% triethyl amine)) to afford (E)-N-(4-(8-(4-chloro-6-ethyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide (Example 22-1, 5.12 mg, single isomer. 1^{st}-eluting isomer) and (E)-N-(4-(8-(4-chloro-6-ethyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide (Example 22-2, 4.87 mg, single isomer, 2^{nd}-eluting isomer).

### Example 23

### (E)-N-(2,6-difluoro-4-(8-(6-hydroxy-1-methyl-1H-benzo[d]imidazol-5-yl)indolizine-3-carbonyl)phenyl)-4-(((1r,4r)-4-methoxycyclohexyl)amino)but-2-enamide

### Step 1

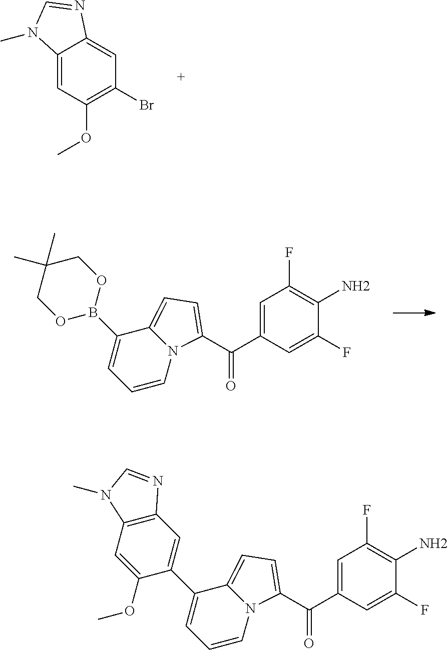 [SMILES: COc1cc2c(cc1Br)ncn2C ; CC1(C)COB(c2cccn3c(C(=O)c4cc(F)c(N)c(F)c4)ccc23)OC1 ; COc1cc2c(cc1-c1cccn3c(C(=O)c4cc(F)c(N)c(F)c4)ccc13)ncn2C]

A solution of 5-bromo-6-methoxy-1-methyl-1H-benzo[d]imidazole (150 mg, 0.622 mmol), (4-amino-3,5-difluorophenyl)(8-(5,5-dimethyl-1,3,2-dioxaborinan-2-yl)indolizin-3-yl)methanone (311 mg, 0.809 mmol), Pd(PPh₃)₄ (72 mg, 0.062 mmol) in 1,4-Dioxane (6.5 mL) and 2M Na₂CO₃ aq. (622 μL, 1.24 mmol) was evacuated and N₂ back-filled before heating to 100° C. for 14 hours After cooling, the reaction mixture was diluted with H₂O and CHCl₃. The resulting suspension was filtered, rinsing with CHCl₃, the filtrate was washed with brine then dried over Na₂SO₄ and evaporated. The residue and filter cake were suspended in EtOAc/hexane, stirred at 50° C. After cooling, the solid was collected by filtration, washing with further hexane, and vacuum-dried at 40° C., thereby obtaining crude (4-amino-3,5-difluorophenyl)(8-(6-methoxy-1-methyl-1H-benzo[d]imidazol-5-yl)indolizin-3-yl)methanone (318 mg, 118%).

### Step 2

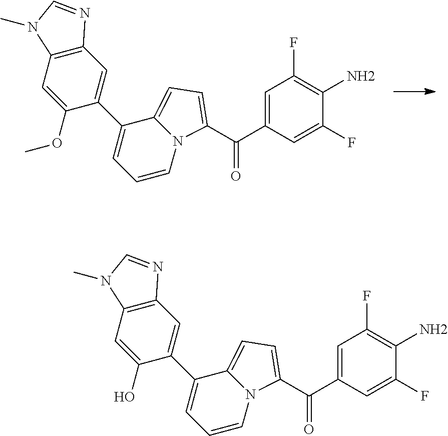 [SMILES: COc1cc2c(cc1-c1cccn3c(C(=O)c4cc(F)c(N)c(F)c4)ccc13)ncn2C ; Cn1cnc2cc(-c3cccn4c(C(=O)c5cc(F)c(N)c(F)c5)ccc34)c(O)cc21]

BBr₃ (1.0 M in CH₂Cl₂, 2.14 mL, 2.14 mmol) was added to a solution of crude (4-amino-3,5-difluorophenyl)(8-(6-methoxy-1-methyl-1H-benzo[d]imidazol-5-yl)indolizin-3-yl)methanone (185 mg, 0.428 mmol) in CH₂Cl₂ (4.28 mL) at 0° C. After stirring at RT for 3 h, the reaction mixture was cooled to 0° C. then sat. NaHCO₃ aq. and CHCl₃/MeOH were added to the reaction mixture slowly. The insoluble solid was filtered and the organic layer was dried over Na2SO4 and evaporated. The residue and filter cake were suspended in MTBE (15 mL), stirred at 50° C. The solid was collected by filtration, washing with further MTBE, and vacuum-dried at 40° C., thereby obtaining crude (4-amino-3,5-difluorophenyl)(8-(6-hydroxy-1-methyl-1H-benzo[d]imidazol-5-yl)indolizin-3-yl)methanone (340 mg, 190%).

### Step 3

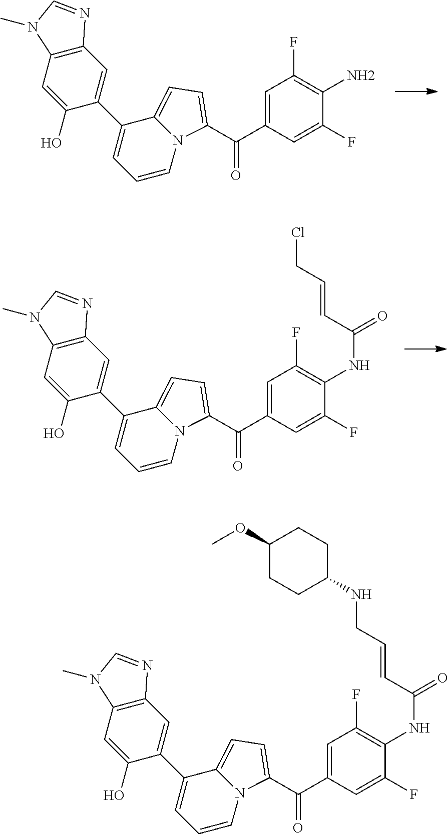 [SMILES: Cn1cnc2cc(-c3cccn4c(C(=O)c5cc(F)c(N)c(F)c5)ccc34)c(O)cc21 ; Cn1cnc2cc(-c3cccn4c(C(=O)c5cc(F)c(NC(=O)/C=C/CCl)c(F)c5)ccc34)c(O)cc21 ; CO[C@H]1CC[C@H](NC/C=C/C(=O)Nc2c(F)cc(C(=O)c3ccc4c(-c5cc6ncn(C)c6cc5O)cccn34)cc2F)CC1]

The procedure of step 3 was performed in the same manner as Example 39 (step 2, 3) to give the title compound.

### Example 24

### (E)-N-(4-(8(1,2-dimethyl-6-(trifluoromethyl)-1H-benzo[d]imidazol-5-yl)indolizine-3-carbonyl)-2,6-difluorophenyl)-4-((tetrahydro-2H-pyran-4-yl)amino)but-2-enamide

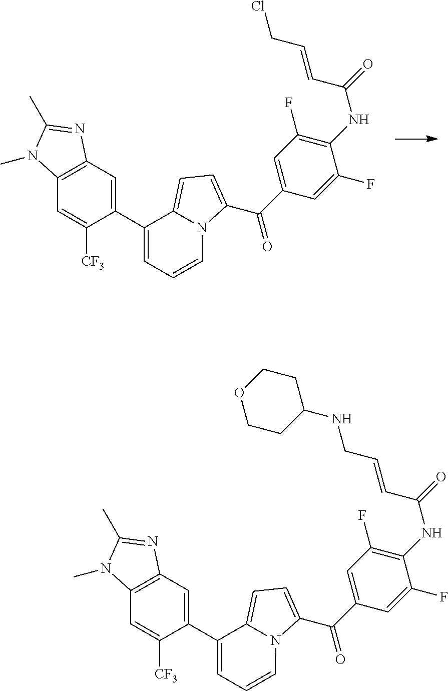 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCOCC6)c(F)c5)ccc34)c(C(F)(F)F)cc2n1C]

A solution of crude (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (170 mg), KI (144 mg, 0.870 mmol) and 4-aminotetrahydropyran (0.150 mL, 1.45 mmol) in DMF (2.0 mL) was stirred at 35° C. for 7 hours. The mixture was diluted with H₂O and EtOAc, and extracted with EtOAc. The organic layer was washed with H₂O and brine, then dried over sodium sulfate, and evaporated. The residue was purified by column chromatography on silica gel (CHCl₃-methanol), thereby obtaining the title compound (71.6 mg).

### Example 25

### (E)-4-(tert-butylamino)-N-(4-(8-(1,2-dimethyl-6-(trifluoromethyl)-1H-benzo[d]imidazol-5-yl)indolizine-3-carbonyl)-2,6-difluorophenyl)but-2-enamide

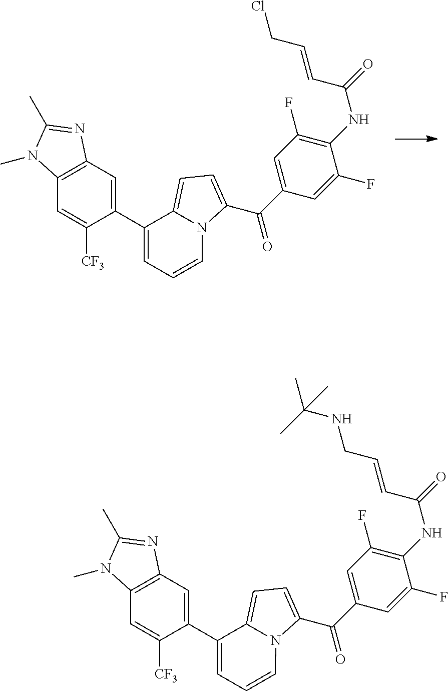 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC(C)(C)C)c(F)c5)ccc34)c(C(F)(F)F)cc2n1C]

The procedure of Example 24 was performed except that tert-butylamine (0.305 mL) was used instead of 4-aminotetrahydropyran in Example 24, thereby obtaining the title compound (74.4 mg).

### Example 26

### (E)-N-(4-(8-(1,2-dimethyl-6-(trifluoromethyl)-1H-benzo[d]imidazol-5-indolizine-3-carbonyl)-2,6-difluorophenyl)-4-((4-methyltetrahydro-2H-pyran-4-yl)amino)but-2-enamide

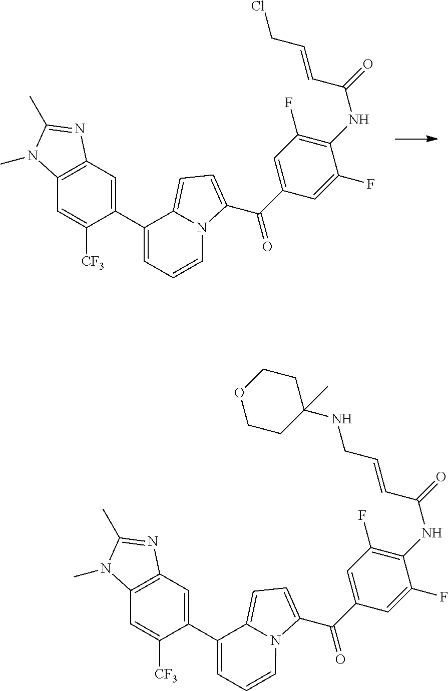 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6(C)CCOCC6)c(F)c5)ccc34)c(C(F)(F)F)cc2n1C]

The procedure of Example 24 was performed except that 4-methyltetrahydropyran-4-amine (0.179 mL) was used instead of 4-aminotetrahydropyran in Example 24, thereby obtaining the title compound (77.8 mag).

### Example 27-1 and 27-2

### (E)-N-(4-(8-(4-chloro-1,6-dimethyl-1H-benzo[d]imidazol-5-yl)indolizine-3-carbonyl)-2,6-difluorophenyl)-4-((tetrahydro-2H-pyran-4-yl)amino)but-2-enamide

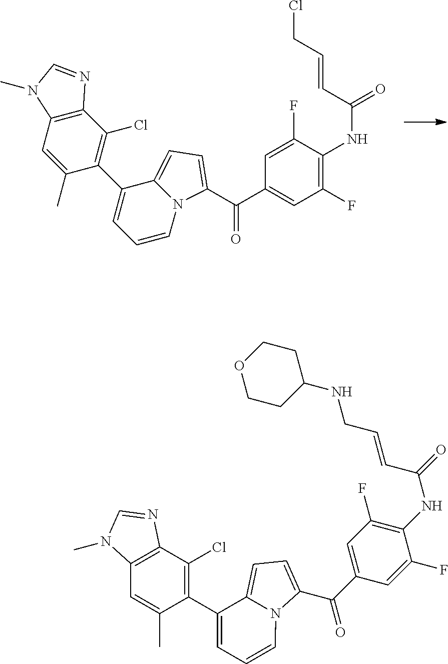 [SMILES: Cc1cc2c(ncn2C)c(Cl)c1-c1cccn2c(C(=O)c3cc(F)c(NC(=O)/C=C/CCl)c(F)c3)ccc12 ; Cc1cc2c(ncn2C)c(Cl)c1-c1cccn2c(C(=O)c3cc(F)c(NC(=O)/C=C/CNC4CCOCC4)c(F)c3)ccc12]

A solution of (E)-4-chloro-N-(4-(8-(4-chloro-1,6-dimethyl-1H-benzo[d]imidazol-5-yl)indolizine-3-carbonyl)-2,6-difluorophenyl)but-2-enamide (68 mg), KI (61.2 mg, 0.369 mmol) and 4-aminotetrahydropyran (0.0318 mL, 0.307 mmol) in DMF (1.0 mL) was stirred at 35° C. for 2.5 hours. To the resulting mixture was added H₂O, and extracted with EtOAc. The organic layer was washed with H₂O and brine, dried over sodium sulfate, and evaporated. The residue was purified by column chromatography on silica gel (CHCl₃-methanol), thereby obtaining the racemic title compound (50.6 mg). The racemate was separated by CHIRAL ART SB (hexane-ethanol (0.1% triethyl amine)) to give the title compound (Example 27-1, 18.4 mg; single isomer. 1^{st}-eluting isomer and Example 27-2, 19.6 mg; single isomer, 2^{nd}-eluting isomer).

### Example 28-1 and 28-2

### (E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide

### Step 1˜3

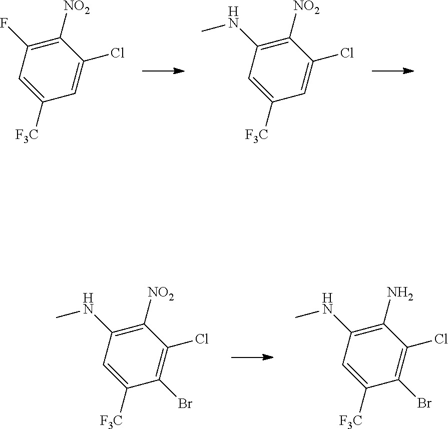 [SMILES: O=[N+]([O-])c1c(F)cc(C(F)(F)F)cc1Cl ; CNc1cc(C(F)(F)F)cc(Cl)c1[N+](=O)[O-] ; CNc1cc(C(F)(F)F)c(Br)c(Cl)c1[N+](=O)[O-] ; CNc1cc(C(F)(F)F)c(Br)c(Cl)c1N]

The procedures of Example 10 (steps 1, 3 and 4) were performed except that 1-chloro-3-fluoro-2-nitro-5-(trifluoromethyl)benzene (7.5 g) was used instead of 5-bromo-1-chloro-3-fluoro-2-nitrobenzene used in Example 10 (step 1), thereby obtaining 4-bromo-3-chloro-N1-methyl-5-(trifluoromethyl)benzene-1,2-diamine (9.4 g).

### Step 4

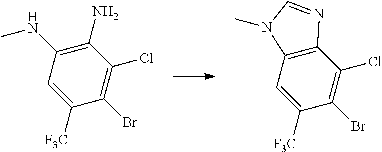 [SMILES: CNc1cc(C(F)(F)F)c(Br)c(Cl)c1N ; Cn1cnc2c(Cl)c(Br)c(C(F)(F)F)cc21]

The procedure of Example 10 (step 5) was performed except that 4-bromo-3-chloro-N1-methyl-5-(trifluoromethyl)benzene-1,2-diamine (600 mg) obtained in step 3 was used instead of 4-bromo-3-chloro-5-ethyl-N1-methylbenzene-1,2-diamine used in Example 10 (step 5), thereby obtaining 5-bromo-4-chloro-1-methyl-6-(trifluoromethyl)-1H-benzo[d]imidazole (538 mg).

### Step 5

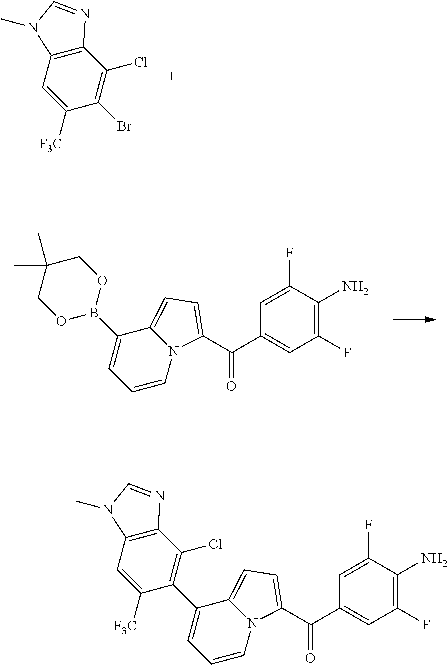 [SMILES: Cn1cnc2c(Cl)c(Br)c(C(F)(F)F)cc21 ; CC1(C)COB(c2cccn3c(C(=O)c4cc(F)c(N)c(F)c4)ccc23)OC1 ; Cn1cnc2c(Cl)c(-c3cccn4c(C(=O)c5cc(F)c(N)c(F)c5)ccc34)c(C(F)(F)F)cc21]

A solution of 5-bromo-4-chloro-1-methyl-6-(trifluoromethyl)-1H-benzo[d]imidazole (65.0 mg) obtained in step 4, (4-amino-3,5-difluorophenyl)(8-(5,5-dimethyl-1,3,2-dioxaborinan-2-yl)indolizin-3-yl)methanone (159 mg), Dichloro[1,3-bis(2,6-Di-3-pentylphenyl)imidazol-2-ylidene](3-chloropyridyl)palladium(II) (4.93 mg), potassium hydroxide (34.9 mg) in 1,4-dioxane (2.0 mL) was stirred at 90° C. for 6 hours. After dilution with ethyl acetate, the mixture was washed with water and a saturated sodium chloride solution, followed by drying over sodium sulfate. After evaporation, the residue was purified by column chromatography (hexane:acetone), thereby obtaining (4-amino-3,5-difluorophenyl)(8-(4-chloro-1-methyl-6-(trifluoromethyl)-1H-benzo[d]imidazol-5-yl)indolizin-3-yl)methanone (30.0 mg).

### Step 6, 7

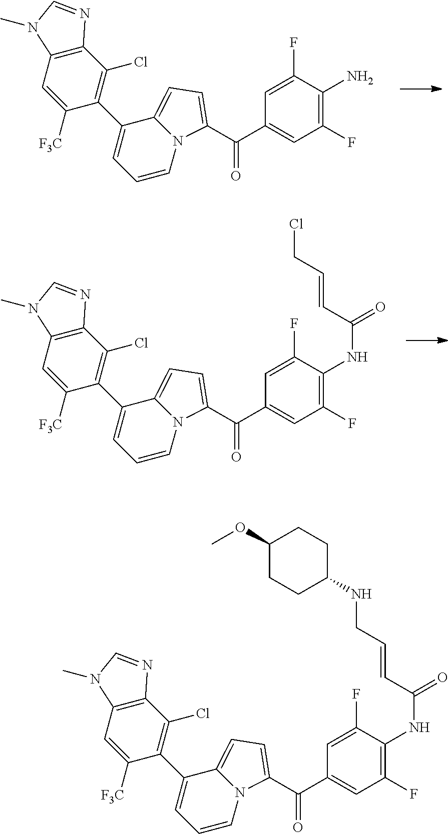 [SMILES: Cn1cnc2c(Cl)c(-c3cccn4c(C(=O)c5cc(F)c(N)c(F)c5)ccc34)c(C(F)(F)F)cc21 ; Cn1cnc2c(Cl)c(-c3cccn4c(C(=O)c5cc(F)c(NC(=O)/C=C/CCl)c(F)c5)ccc34)c(C(F)(F)F)cc21 ; CO[C@H]1CC[C@H](NC/C=C/C(=O)Nc2c(F)cc(C(=O)c3ccc4c(-c5c(C(F)(F)F)cc6c(ncn6C)c5Cl)cccn34)cc2F)CC1]

The procedure of Example 10 (steps 7 and 8) was performed except that (4-amino-3,5-difluorophenyl)(8-(4-chloro-1-methyl-6-(trifluoromethyl)-1H-benzo[d]imidazol-5-yl)indolizin-3-yl)methanone (30.0 mg) obtained in step 5 was used instead of (4-amino-3,5-difluorophenyl)(8-(4-chloro-6-ethyl-1-methyl-1H-benzo[d]imidazol-5-yl)indolizin-3-yl)methanone used in Example 10 (step 7), thereby obtaining title compound (12.0 mg).

### Step 8

(E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (11.0 mg) obtained in step 7 was dissolved in EtOH and purified by preparative HPLC (CHIRAL ART SB, hexane-ethanol (0.1% triethyl amine)) to afford (E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (Example 28-1, 4.36 mg, single isomer, 1^{st}-eluting isomer) and (E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (Example 28-2, 4.44 mg, single isomer. 2^{nd}-eluting isomer).

### Example 29

### (E)-4-(tert-butylamino)-N-(2,6-difluoro-4-(8-(1,4,6-trimethyl-1H-benzo[d]imidazol-5-vi)indolizine-3-carbonyl)phenyl)but-2-enamide

The title compound was obtained using the same procedure as that of other examples.

### Example 30-1 and 30-2

### (E)-N-(4-(8-(2-chloro-6-methoxy-4-(2-oxopyrrolidin-1-yl)phenyl)indolizine-3-carbonyl)-2,6-difluorophenyl)-4-(((1r,4r)-4-methoxycyclohexyl)amino)but-2-enamide

### Step 1

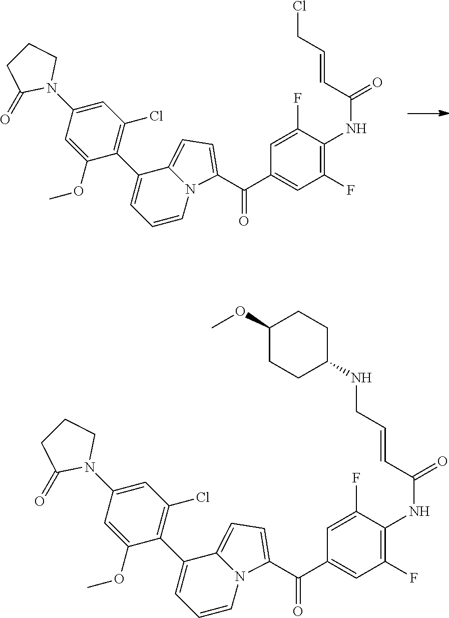 [SMILES: COc1cc(N2CCCC2=O)cc(Cl)c1-c1cccn2c(C(=O)c3cc(F)c(NC(=O)/C=C/CCl)c(F)c3)ccc12 ; COc1cc(N2CCCC2=O)cc(Cl)c1-c1cccn2c(C(=O)c3cc(F)c(NC(=O)/C=C/CN[C@H]4CC[C@H](OC)CC4)c(F)c3)ccc12]

The procedure of Example 10 (step 8) was performed except that (E)-4-chloro-N-(4-(8-(2-chloro-6-methoxy-4-(2-oxopyrrolidin-1-yl)phenyl)indolizine-3-carbonyl)-2,6-difluorophenyl)but-2-enamide (65.0 mg) obtained in step 7 (Example 21) was used instead of (E)-4-chloro-N-(4-(8-(4-chloro-6-ethyl-1-methyl-1H-benzo[d]imidazol-5-yl)indolizine-3-carbonyl)-2,6-difluorophenyl)but-2-enamide used in Example 10 (step 8), thereby obtaining the title compound (47.0 mg).

### Step 2

(E)-N-(4-(8-(2-chloro-6-methoxy-4-(2-oxopyrrolidin-1-yl)phenyl)indolizine-3-carbonyl)-2,6-difluorophenyl)-4-(((1r,4r)-4-methoxycyclohexyl)amino)but-2-enamide (42.0 mg) obtained in step 1 was dissolved in EtOH and purified by preparative HPLC (CHIRALPAK IA, hexane-ethanol (0.1% triethyl amine)) to afford (E)-N-(4-(8-(2-chloro-6-methoxy-4-(2-oxopyrrolidin-1-yl)phenyl)indolizine-3-carbonyl)-2,6-difluorophenyl)-4-(((1r,4r)-4-methoxycyclohexyl)amino)but-2-enamide (Example 30-1, 17.5 mg, single isomer. 1^{st}-eluting isomer) and (E)-N-(4-(8-(2-chloro-6-methoxy-4-(2-oxopyrrolidin-1-yl)phenyl)indolizine-3-carbonyl)-2,6-difluorophenyl)-4-(((1r,4r)-4-methoxycyclohexyl)amino)but-2-enamide (Example 30-2, 17.3 mg, single isomer, 2^{nd}-eluting isomer).

### Example 31

### (E)-N-(4-(8(6-bromo-4-chloro-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

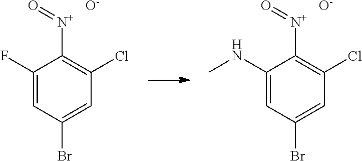

Methylamine (2M THF solution. 33.1 mL, 66.3 mmol) was added to a solution of 5-bromo-1-chloro-3-fluoro-2-nitrobenzene (5.62 g, 22.1 mmol) in iPrOH (30.0 mL). After stirring at ambient temperature for 20 minutes, the reaction mixture was concentrated under reduced pressure, thereby obtaining crude 5-bromo-3-chloro-N-methyl-2-nitroaniline (5.86 g. 66.3 mmol).

### Step 2

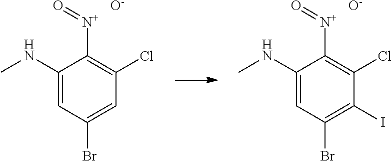

N-Iodosuccinimide (5.00 g, 22.2 mmol) was added to a solution of 5-bromo-3-chloro-N-methyl-2-nitroaniline (5.62 g, 21.2 mmol) in acetic acid (70.0 mL). After the mixture was stirred at 70° C. for 90 minutes, the mixture was cooled to ambient temperature and water (150 mL) was added to reaction mixture. The resulting solid was collected by filtration, and vacuum-dried at 50° C. for 1 day, thereby obtaining crude 5-bromo-3-chloro-4-iodo-N-methyl-2-nitroaniline (8.27 g, 20.7 mmol).

### Step 3

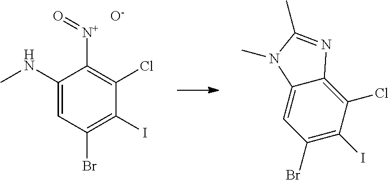

Acetic anhydride (13.2 mL, 140 mmol) was added to 5-bromo-3-chloro-4-iodo-N-methyl-2-nitroaniline (3.00 g, 7.67 mmol). After the mixture was stirred at 180° C. for 2 hours, the mixture was cooled to ambient temperature. EtOAc and saturated aqueous NaHCO₃, and extracted with EtOAc, and washed successively with H₂O then brine, dried over sodium sulfate, and evaporated. The residue was purified by column chromatography on silica gel (EtOAc-hexane), thereby obtaining N-(5-bromo-3-chloro-4-iodo-2-nitrophenyl)-N-methylacetamide.

Iron (3.00 g. 53.7 mmol) was added to a solution of N-(5-bromo-3-chloro-4-iodo-2-nitrophenyl)-N-methylacetamide (3.32 g, 7.67 mmol) in acetic acid (30.0 mL). After the mixture was stirred at 85° C. for 40 minutes, the mixture was cooled to ambient temperature. EtOAc was added to the reaction mixture and insoluble matter was filtered off through celite pad and the mixture was extracted with EtOAc and H₂O, and washed successively with H₂O then brine, dried over sodium sulfate, and evaporated. The residue was purified by column chromatography on silica gel (methanol-EtOAc-hexane), thereby obtaining 6-bromo-4-chloro-5-iodo-1,2-dimethyl-1H-benzo[d]imidazole (2.01 g, 5.24 mmol).

### Step 4

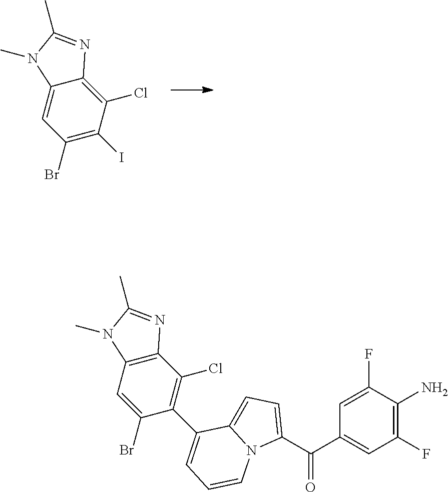 [SMILES: Cc1nc2c(Cl)c(I)c(Br)cc2n1C ; Cc1nc2c(Cl)c(-c3cccn4c(C(=O)c5cc(F)c(N)c(F)c5)ccc34)c(Br)cc2n1C]

To a mixture of 6-bromo-4-chloro-5-iodo-1,2-dimethyl-1H-benzo[d]imidazole (1.00 g, 2.59 mmol), (4-amino-3,5-difluorophenyl)(8-(5,5-dimethyl-1,3,2-dioxaborinan-2-yl)indolizin-3-yl)methanone (1.40 g. 3.63 mmol) and Pd(PPh3)4 (300 mg, 0.261 mmol) in 1,2-dimethoxyethane (10.0 mL) was added 2M aqueous sodium carbonate solution (2.60 mL, 5.19 mmol). After reacting by microwave apparatus at 120° C. for 5 hours, the mixture was diluted with EtOAc and water. The organic layer was washed successively with H₂O then brine, dried over sodium sulfate, and evaporated. The residue was purified by column chromatography on silica gel (acetone-hexane), thereby obtaining (4-amino-3,5-difluorophenyl)(8-(6-bromo-4-chloro-1,2-dimethyl-1H-benzo[d]imidazol-5-yl)indolizin-3-yl)methanone (0.942 g, 1.78 mmol).

### Step 5

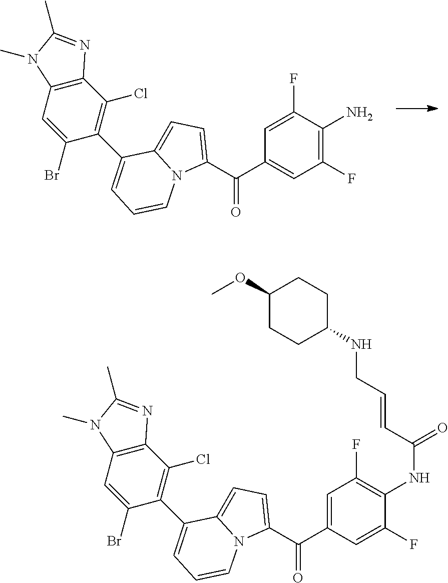 [SMILES: Cc1nc2c(Cl)c(-c3cccn4c(C(=O)c5cc(F)c(N)c(F)c5)ccc34)c(Br)cc2n1C ; CO[C@H]1CC[C@H](NC/C=C/C(=O)Nc2c(F)cc(C(=O)c3ccc4c(-c5c(Br)cc6c(nc(C)n6C)c5Cl)cccn34)cc2F)CC1]

A solution of (4-amino-3,5-difluorophenyl)(8-(6-bromo-4-chloro-1,2-dimethyl-1H-benzo[d]imidazol-5-yl)indolizin-3-yl)methanone (20.4 mg, 0.0382 mmol). 4-chlorocrotonic acid (9.12 mg, 0.0761 mmol) and propylphosphonic anhydride solution (50 wt % in THF. 71.0 μL, 0.115 mmol) in DMF (250 μL) was stirred at ambient temperature for 15 minutes. Then triethylamine (32.8 μL, 0.226 mmol) was added thereto, and the mixture was stirred at 35° C. for 1 hour. To the resulting mixture was added saturated aqueous NaHCO₃, and extracted with EtOAc. and washed successively with H₂O then brine, dried over sodium sulfate, and evaporated. The residue was used for next reaction without further purification.

A solution of (E)-N-(4-(8-(6-bromo-4-chloro-1,2-dimethyl-1H-benzo[d]imidazol-5-yl)indolizine-3-carbonyl)-2,6-difluorophenyl)-4-chlorobut-2-enamide (24.2 mg, 0.0382 mmol), trans-4-methoxycyclohexanamine hydrochloride (13.5 mg, 0.0766 mmol), KI (19.3 mg, 0.117 mmol) and K₃PO₄ (32.2 mg, 0.152 mmol) in DMF (500 μL) was stirred at ambient temperature for 1 day. To the resulting mixture was added saturated aqueous NaHCO₃, and extracted with EtOAc. and washed successively with H₂O then brine, dried over sodium sulfate, and evaporated. The residue was purified by preparative HPLC (water:acetonitrile (0.1% formic acid)) to give the title compound (3.86 mg, 0.00533 mmol).

### Example 32-1 and 32-2

### (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide

### Step 1

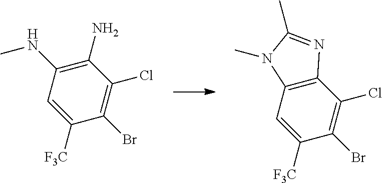 [SMILES: CNc1cc(C(F)(F)F)c(Br)c(Cl)c1N ; Cc1nc2c(Cl)c(Br)c(C(F)(F)F)cc2n1C]

The procedure of Example 10 (step 5) was performed except that 4-bromo-3-chloro-N1-methyl-5-(trifluoromethyl)benzene-1,2-diamine (220 mg) obtained in Example 28 (step 3) and 1,1,1-triethoxyethane were used instead of 4-bromo-3-chloro-5-ethyl-N1-methylbenzene-1,2-diamine used in Example 10 (step 5) and triethoxymethane, thereby obtaining 5-bromo-4-chloro-1,2-dimethyl-6-(trifluoromethyl)-1H-benzo[d]imidazole (210 mg).

### Step 2

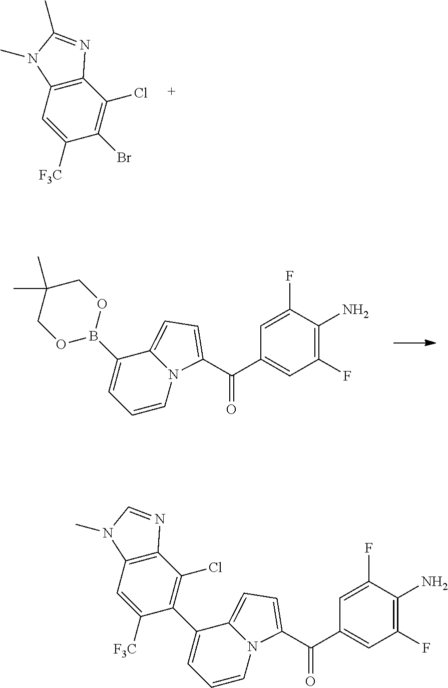 [SMILES: Cc1nc2c(Cl)c(Br)c(C(F)(F)F)cc2n1C ; CC1(C)COB(c2cccn3c(C(=O)c4cc(F)c(N)c(F)c4)ccc23)OC1 ; Cn1cnc2c(Cl)c(-c3cccn4c(C(=O)c5cc(F)c(N)c(F)c5)ccc34)c(C(F)(F)F)cc21]

A solution of 5-bromo-4-chloro-1,2-dimethyl-6-(trifluoromethyl)-1H-benzo[d]imidazole (150 mg) obtained in step 1. (4-amino-3,5-difluorophenyl)(8-(5,5-dimethyl-1,3,2-dioxaborinan-2-yl)indolizin-3-yl)methanone (352 mg), dichloro[1,3-bis(2,6-Di-3-pentylphenyl)imidazol-2-ylidene](3-chloropyridyl)palladium(II) (10.9 mg), potassium hydroxide (77.1 mg) in 1,4-dioxane (5.0 mL) was stirred at 110° C. for 3 hours. After dilution with ethyl acetate, the mixture was washed with water and a saturated sodium chloride solution, followed by drying over sodium sulfate. After evaporation, the residue was purified by column chromatography (hexane:acetone), thereby obtaining (4-amino-3,5-difluorophenyl)(8-(4-chloro-1,2-dimethyl-6-(trifluoromethyl)-1H-benzo[d]imidazol-5-yl)indolizin-3-yl)methanone (123 mg).

### Step 3, 4

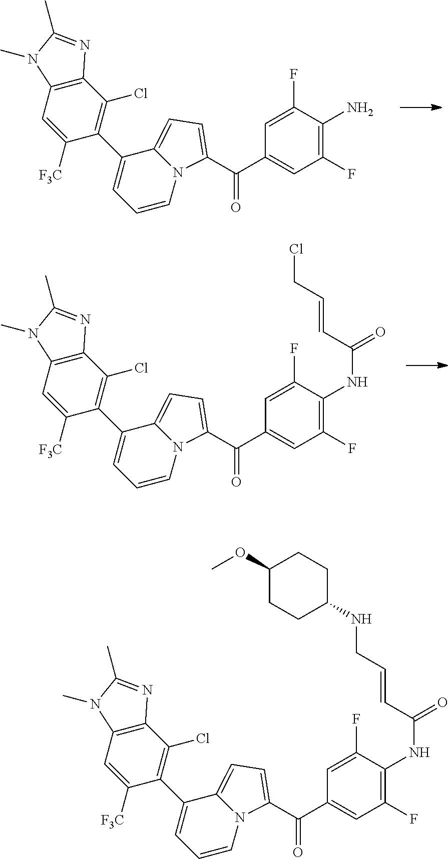 [SMILES: Cc1nc2c(Cl)c(-c3cccn4c(C(=O)c5cc(F)c(N)c(F)c5)ccc34)c(C(F)(F)F)cc2n1C ; Cc1nc2c(Cl)c(-c3cccn4c(C(=O)c5cc(F)c(NC(=O)/C=C/CCl)c(F)c5)ccc34)c(C(F)(F)F)cc2n1C ; CO[C@H]1CC[C@H](NC/C=C/C(=O)Nc2c(F)cc(C(=O)c3ccc4c(-c5c(C(F)(F)F)cc6c(nc(C)n6C)c5Cl)cccn34)cc2F)CC1]

The procedure of Example 10 (steps 7 and 8) was performed except that (4-amino-3,5-difluorophenyl)(8-(4-chloro-1,2-dimethyl-6-(trifluoromethyl)-1H-benzo[d]imidazol-5-yl)indolizin-3-yl)methanone (161 mg) obtained in step 2 was used instead of (4-amino-3,5-difluorophenyl)(8-(4-chloro-6-ethyl-1-methyl-1H-benzo[d]imidazol-5-yl)indolizin-3-yl)methanone used in Example 10 (step 7), thereby obtaining title compound (75.0 mg).

### Step 5

(E)-N-(4-(8-(4-chloro-1,2-dimethyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (72.0 mg) obtained in step 4 was dissolved in EtOH and purified by preparative HPLC (CHIRAL AR T SB, hexane-ethanol (0.1% triethyl amine)) to afford (E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (Example 32-1, 33.0 mg, single isomer, 1^{st}-eluting isomer) and (E)-N-(4-(8-(4-chloro-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide (Example 32-2, 32.0 mg, single isomer. 2^{nd}-eluting isomer).

### Example 33

### (E)-N-(2,6-difluoro-4-(8-(5-methoxy-2,3-dimethyl-3H-imidazo[4,5-b]pyridin-6-yl)indolizine-3-carbonyl)phenyl)-4-(((1r,4r)-4-methoxycyclohexyl)amino)but-2-enamide

### Step 1, 2

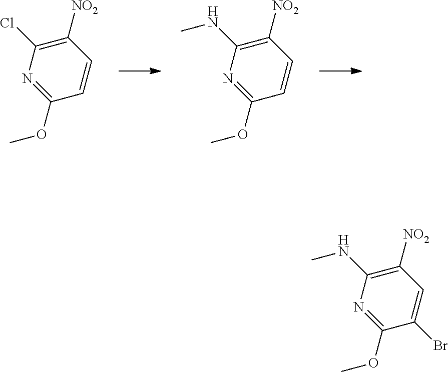

The procedures of Example 10 (steps 1, 3) were performed except that 2-chloro-6-methoxy-3-nitropyridine (10.0 g) was used instead of 5-bromo-1-chloro-3-fluoro-2-nitrobenzene used in Example 10 (step 1), thereby obtaining 5-bromo-6-methoxy-N-methyl-3-nitropyridin-2-amine (12.7 g).

### Step 3

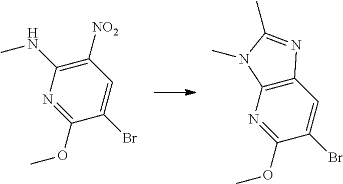

A solution of 5-bromo-6-methoxy-N-methyl-3-nitropyridin-2-amine (2.5 g) obtained in step 2 in acetic anhydride was stirred at 180° C. for 3 hours using microwave irradiation. To the reaction mixture, iron powder (1.6 g) and acetic acid (10 mL) were added, followed by stirring at 90° C. for 2 hours. To the reaction mixture, zinc powder (1.9 g) and conc. hydrochloric acid (3.5 mL) were added, followed by stirring at 90° C. for 2 hours. The insoluble matter was filtered off, followed by evaporating the solvent under reduced pressure. After dilution with ethyl acetate, the mixture was washed with a saturated sodium bicarbonate solution and a saturated sodium chloride solution, followed by drying over sodium sulfate. After evaporation, the residue was purified by column chromatography (chloroform:ethanol), thereby obtaining 6-bromo-5-methoxy-2,3-dimethyl-3H-imidazo[4,5-b]pyridine (300 mg).

### Step 4

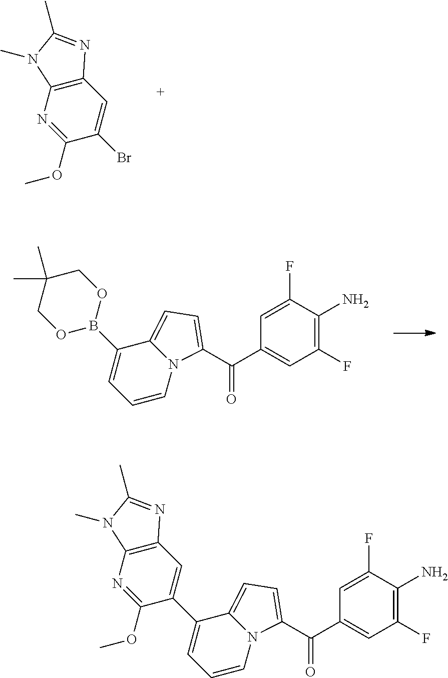 [SMILES: COc1nc2c(cc1Br)nc(C)n2C ; CC1(C)COB(c2cccn3c(C(=O)c4cc(F)c(N)c(F)c4)ccc23)OC1 ; COc1nc2c(cc1-c1cccn3c(C(=O)c4cc(F)c(N)c(F)c4)ccc13)nc(C)n2C]

A solution of 6-bromo-5-methoxy-2,3-dimethyl-3H-imidazo[4,5-b]pyridine (53.1 mg) obtained in step 3, (4-amino-3,5-difluorophenyl)(8-(5,5-dimethyl-1,3,2-dioxaborinan-2-yl)indolizin-3-yl)methanone (159 mg), tetrakis(triphenylphosphine)palladium(0) (24.0 mg). 2.0 M sodium bicarbonate in water (207 μL) in 1,2-dimethoxyethane (1.5 mL) was stirred at 140° C. for 40 minutes using microwave irradiation. After dilution with ethyl acetate, the mixture was washed with a saturated sodium chloride solution, followed by drying over sodium sulfate. After evaporation, the residue was purified by column chromatography (chloroform:ethanol), thereby obtaining (4-amino-3,5-difluorophenyl)(8-(5-methoxy-2,3-dimethyl-3H-imidazo[4,5-b]pyridin-6-yl)indolizin-3-yl)methanone (72.1 mg).

### Step 5

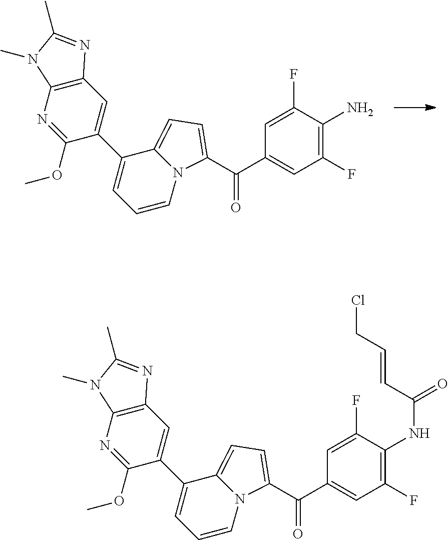 [SMILES: COc1nc2c(cc1-c1cccn3c(C(=O)c4cc(F)c(N)c(F)c4)ccc13)nc(C)n2C ; COc1nc2c(cc1-c1cccn3c(C(=O)c4cc(F)c(NC(=O)/C=C/CCl)c(F)c4)ccc13)nc(C)n2C]

(E)-4-chlorobut-2-enoic acid (38.8 mg) and 1-propanephosphonic acid anhydride cyclic trimer (a 1.7M THF solution. 302 μL) were added to a solution of (4-amino-3,5-difluorophenyl)(8-(5-methoxy-2,3-dimethyl-3H-imidazo[4,5-b]pyridin-6-yl)indolizin-3-yl)methanone (72.1 mg) obtained in step 4 in DMF (1.4 mL), followed by stirring at room temperature for 20 minutes. Triethylamine (134 μL) was added to the reaction mixture. A reaction was performed at room temperature for 2 hours. After water was added to the reaction mixture, extraction was performed with ethyl acetate, followed by washing the organic layer with a saturated sodium chloride solution. After the washed layer was dried over sodium sulfate, the solvent was evaporated under reduced pressure. The obtained residue was purified by column chromatography (chloroform:ethanol), thereby obtaining (E)-4-chloro-N-(2,6-difluoro-4-(8-(5-methoxy-2,3-dimethyl-3H-imidazo[4,5-b]pyridin-6-yl)indolizine-3-carbonyl)phenyl)but-2-enamide (70.6 mg).

### Step 6

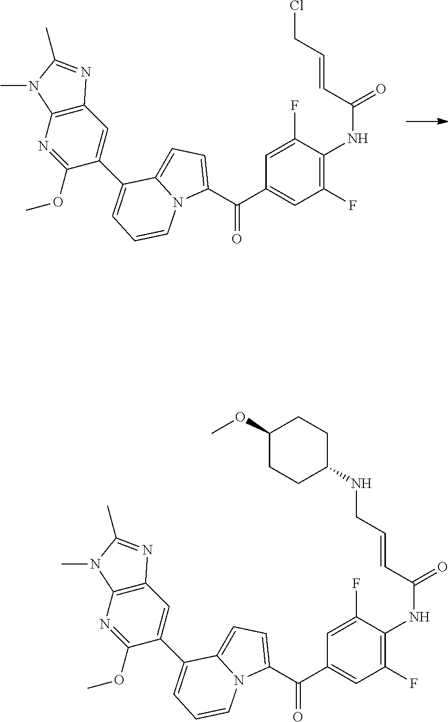 [SMILES: COc1nc2c(cc1-c1cccn3c(C(=O)c4cc(F)c(NC(=O)/C=C/CCl)c(F)c4)ccc13)nc(C)n2C ; COc1nc2c(cc1-c1cccn3c(C(=O)c4cc(F)c(NC(=O)/C=C/CN[C@H]5CC[C@H](OC)CC5)c(F)c4)ccc13)nc(C)n2C]

(1r,4r)-4-methoxycyclohexan-1-amine hydrochloride (21.1 mg), tripotassium phosphate (54.0 mg), and potassium iodide (31.7 mg) were added to a solution of (E)-4-chloro-N-(2,6-difluoro-4-(8-(5-methoxy-2,3-dimethyl-3H-imidazo[4,5-b]pyridin-6-yl)indolizine-3-carbonyl)phenyl)but-2-enamide (35.0 mg) obtained in step 5 in DMF (600 μL). A reaction was performed at room temperature for 3 hours. Water was added to the reaction mixture, and extraction was performed with ethyl acetate, followed by washing the organic layer with a saturated sodium chloride solution. The washed layer was dried over sodium sulfate, and the solvent was evaporated under reduced pressure. The residue was purified by preparative reversed-phase HPLC (water:acetonitrile (0.1% formic acid)). Saturated sodium bicarbonate water was added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (12.9 mg).

### Example 34-1 and 34-2

### (E)-N-(2,6-difluoro-4-(8-(1,4,6-trimethyl-1H-benzo[d]imidazol-5-yl)indolizine-3-carbonyl)phenyl)-4-(((1r,4r)-4-methoxycyclohexyl)amino)but-2-enamide

The title compounds were obtained using the same procedure as that of other examples. The racemate was dissolved in EtOH and purified by preparative HPLC (CHIRAL ART SB, hexane-ethanol (0.1% triethyl amine)) to afford (E)-N-(2,6-difluoro-4-(8-(1,4,6-trimethyl-1H-benzo[d]imidazol-5-yl)indolizine-3-carbonyl)phenyl)-4-(((1r,4r)-4-methoxycyclohexyl)amino)but-2-enamide (Example 34-1, 14.1 mg, single isomer, 1^{st}-eluting isomer) and (E)-N-(2,6-difluoro-4-(8-(1,4,6-trimethyl-1H-benzo[d]imidazol-5-yl)indolizine-3-carbonyl)phenyl)-4-(((1r,4r)-4-methoxycyclohexyl)amino)but-2-enamide (Example 34-2, 14.4 mg, single isomer, 2^{nd}-eluting isomer).

### Example 35-1 and 35-2

### (E)-N-(4-(8-(4-chloro-6-methoxy-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

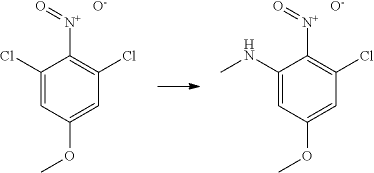

Methylamine (2M THF solution. 6.75 mL, 13.5 mmol) was added to a solution of 1,3-dichloro-5-methoxy-2-nitrobenzene (1.50 g, 6.75 mmol) in DMSO (2.50 mL). After reacting by microwave apparatus at 80° C. for 3 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining 3-chloro-5-methoxy-N-methyl-2-nitroaniline (0.369 g. 1.66 mmol).

### Step 2

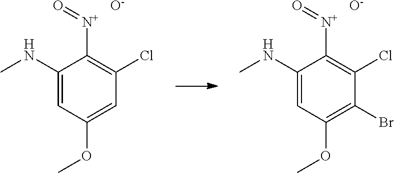

To a solution of 3-chloro-5-methoxy-N-methyl-2-nitroaniline (364 mg, 1.68 mmol) in THF (5.60 mL) was added N-Bromosuccinimide (329 mg, 1.85 mmol) at ambient temperature, and the mixture was stirred for 20 minutes. Saturated aqueous NaHCO₃ aq., Na₂S2O₃ and EtOAc were added to the reaction mixture. The organic layer was separated, washed with brine, dried over Na₂SO₄, and evaporated, thereby obtaining crude 4-bromo-3-chloro-5-methoxy-N-methyl-2-nitroaniline (561 mg, 1.91 mmol).

### Step 3

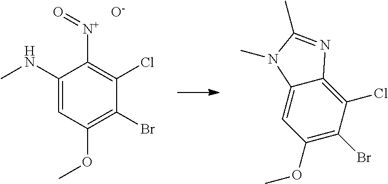

Acetic anhydride (5.21 mL, 54.1 mmol) was added to 4-bromo-3-chloro-5-methoxy-N-methyl-2-nitroaniline (2.00 g. 6.77 mmol). After the mixture was stirred at 180° C. for 2.5 hours, the mixture was cooled to ambient temperature. EtOAc and saturated aqueous NaHCO₃ were added to the reaction mixture. The organic layer was extracted with EtOAc, and washed successively with H₂O then brine, dried over sodium sulfate, and evaporated. The residue was purified by column chromatography on silica gel (EtOAc-hexane), thereby obtaining N-(3-chloro-5-methoxy-2-nitrophenyl)-N-methylacetamide (2.10 g, 6.22 mmol).

Iron (2.65 g. 47.4 mmol) was added to a solution of N-(3-chloro-5-methoxy-2-nitrophenyl)-N-methylacetamide (2.10 g. 6.22 mmol) in acetic acid (10.0 mL). After the mixture was stirred at 85° C. for 1 hour, the mixture was cooled to ambient temperature. EtOAc was added to the reaction mixture and insoluble matter was filtered off through celite pad and the mixture was extracted with EtOAc and H₂O, and washed successively with H₂O then brine, dried over sodium sulfate, and evaporated. The residue was purified by column chromatography on silica gel (methanol-EtOAc-hexane), thereby obtaining 5-bromo-4-chloro-6-methoxy-1,2-dimethyl-1H-benzo[d]imidazole (1.48 g, 5.11 mmol).

### Step 4

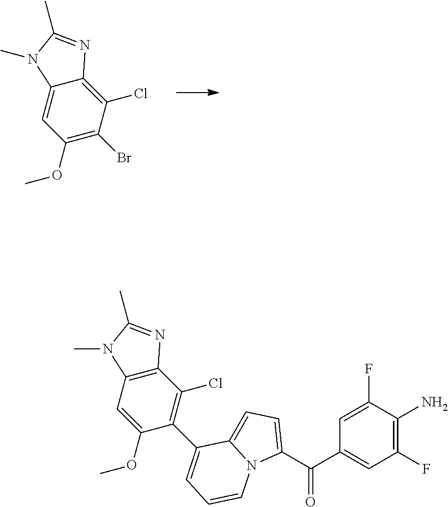 [SMILES: COc1cc2c(nc(C)n2C)c(Cl)c1Br ; COc1cc2c(nc(C)n2C)c(Cl)c1-c1cccn2c(C(=O)c3cc(F)c(N)c(F)c3)ccc12]

To a mixture of 5-bromo-4-chloro-6-methoxy-1,2-dimethyl-1H-benzo[d]imidazole (0.79 g. 2.73 mmol), (4-amino-3,5-difluorophenyl)(8-(5,5-dimethyl-1,3,2-dioxaborinan-2-yl)indolizin-3-yl)methanone (3.15 g, 8.18 mmol) and Pd(PPh3)4 (473 mg, 0.414 mmol) in 1,2-dimethoxyethane (18.2 mL) was added 2M aqueous sodium carbonate solution (6.82 mL, 13.6 mmol). After reacting by microwave apparatus at 120° C. for 3 hours, the mixture was diluted with EtOAc and H₂O. The organic layer was washed successively with H₂O then brine, dried over sodium sulfate, and evaporated. The residue was purified by column chromatography on silica gel (EtOAc-hexane), thereby obtaining (4-amino-3,5-difluorophenyl)(8-(4-chloro-6-methoxy-1,2-dimethyl-1H-benzo[d]imidazol-5-yl)indolizin-3-yl)methanone (1.13 g, 2.35 mmol).

### Step 5

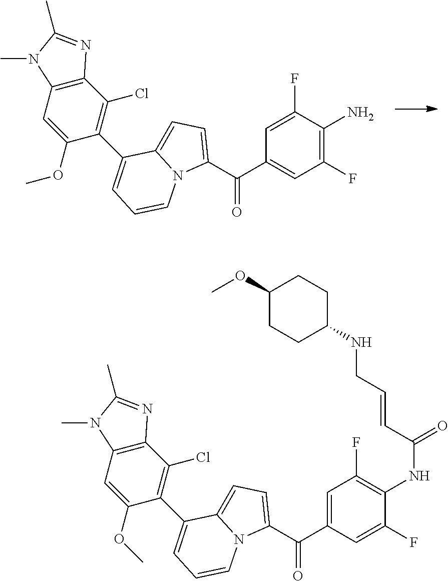 [SMILES: COc1cc2c(nc(C)n2C)c(Cl)c1-c1cccn2c(C(=O)c3cc(F)c(N)c(F)c3)ccc12 ; COc1cc2c(nc(C)n2C)c(Cl)c1-c1cccn2c(C(=O)c3cc(F)c(NC(=O)/C=C/CN[C@H]4CC[C@H](OC)CC4)c(F)c3)ccc12]

A solution of (4-amino-3,5-difluorophenyl)(8-(4-chloro-6-methoxy-1,2-dimethyl-1H-benzo[d]imidazol-5-yl)indolizin-3-yl)methanone (309 mg, 0.643 mmol). 4-chlorocrotonic acid (155 mg, 1.29 mmol) and propylphosphonic anhydride solution (50 wt % in THF. 1.20 mL, 1.93 mmol) in DMF (4.30 mL) was stirred at ambient temperature for 20 minutes. Then triethylamine (535 μL, 3.86 mmol) was added thereto, and the mixture was stirred at 35° C. for 30 minutes. To the resulting mixture was added saturated aqueous NaHCO₃, and extracted with EtOAc. and washed successively with H₂O then brine, dried over sodium sulfate, and evaporated. The residue was used for next reaction without further purification.

A solution of (E)-4-chloro-N-(4-(8-(4-chloro-6-methoxy-1,2-dimethyl-1H-benzo[d]imidazol-5-yl)indolizine-3-carbonyl)-2,6-difluorophenyl)but-2-enamide (187 mg. 0.320 mmol), trans-4-methoxycyclohexanamine hydrochloride (106 mg, 0.640 mmol). KI (159 g, 0.960 mmol) and K₃PO₄ (272 g. 1.28 mmol) in DMF (2.00 mL) was stirred at 35° C. for 1 hour. To the resulting mixture was added saturated aqueous NaHCO₃, and extracted with EtOAc. and washed successively with H₂O then brine, dried over sodium sulfate, and evaporated. The residue was purified by column chromatography on silica gel (methanol-EtOAc-hexane), thereby obtaining the racemate title compound (133 mg, 0.197 mmol). The racemate was separated by CHIRALPAK ART SB(YMC) (hexane-ethanol(0.1% triethyl amine)) to give the title compound (32.6 mg, single isomer, 1-eluting isomer) and (31.1 mg, single isomer, 2^{nd}-eluting isomer).

### Example 36-1 and 36-2

### (E)-4-(tert-butylamino)-N-(4-(8-(4-chloro-6-methoxy-1,2-dimethyl-1H-benzo[d]imidazol-5-yl)indolizine-3-carbonyl)-2,6-difluorophenyl)but-2-enamide

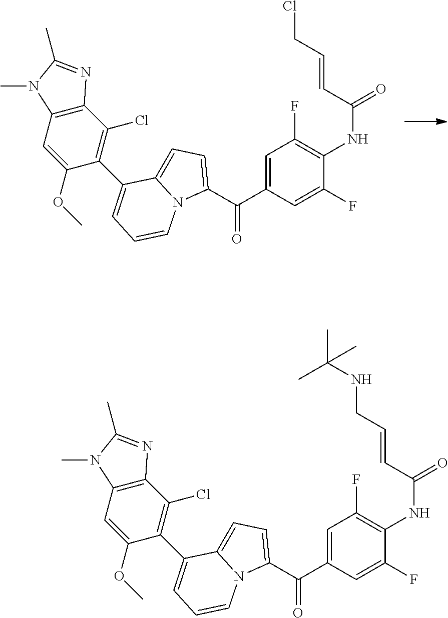 [SMILES: COc1cc2c(nc(C)n2C)c(Cl)c1-c1cccn2c(C(=O)c3cc(F)c(NC(=O)/C=C/CCl)c(F)c3)ccc12 ; COc1cc2c(nc(C)n2C)c(Cl)c1-c1cccn2c(C(=O)c3cc(F)c(NC(=O)/C=C/CNC(C)(C)C)c(F)c3)ccc12]

A solution of (E)-4-chloro-N-(4-(8-(4-chloro-6-methoxy-1,2-dimethyl-1H-benzo[d]imidazol-5-yl)indolizine-3-carbonyl)-2,6-difluorophenyl)but-2-enamide (187 mg, 0.320 mmol), 2-methylpropan-2-amine (67.3 μL, 0.642 mmol), and KI (159 mg, 0.965 mmol) in DMF (2.00 mL) was stirred at 35° C. for 1 hour. To the resulting mixture was added saturated aqueous NaHCO₃, and extracted with EtOAc, and washed successively with H₂O then brine, dried over sodium sulfate, and evaporated. The residue was purified by column chromatography on silica gel (methanol-EtOAc-hexane), thereby obtaining the racemate title compound (67.5 mg, 0.113 mmol). The racemate was separated by CHIRALPAK ID (hexane-ethanol(0.1/triethyl amine)) to give the title compound (16.5 mg, single isomer. 1^{st}-eluting isomer) and (15.1 mg, single isomer, 2^{nd}-eluting isomer).

### Example 37

### (E)-N-(4-(8-(4-chloro-6-cyano-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

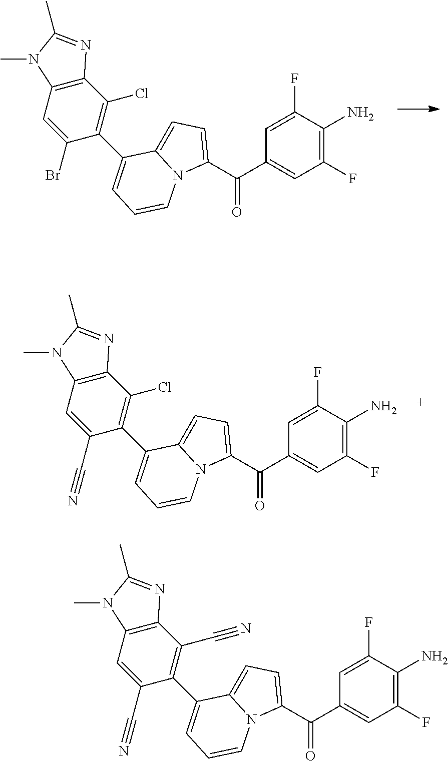 [SMILES: Cc1nc2c(Cl)c(-c3cccn4c(C(=O)c5cc(F)c(N)c(F)c5)ccc34)c(Br)cc2n1C ; Cc1nc2c(Cl)c(-c3cccn4c(C(=O)c5cc(F)c(N)c(F)c5)ccc34)c(C#N)cc2n1C ; Cc1nc2c(C#N)c(-c3cccn4c(C(=O)c5cc(F)c(N)c(F)c5)ccc34)c(C#N)cc2n1C]

To a mixture of (4-amino-3,5-difluorophenyl)(8-(6-bromo-4-chloro-1,2-dimethyl-1H-benzo[d]imidazol-5-yl)indolizin-3-yl)methanone (100 mg, 0.189 mmol), and Pd(dppf)Cl2 (27.6 mg, 0.0378 mmol) in DMF (500 μL) was added zinc cyanide (33.3 mg, 0.283 mmol). After reacting by microwave apparatus at 150° C. for 1 hour, the mixture was diluted with EtOAc and water. The organic layer was washed successively with H₂O then brine, dried over sodium sulfate, and evaporated. The residue was purified by column chromatography on silica gel (EtOAc-hexane), thereby obtaining mixture of 5-(3-(4-amino-3,5-difluorobenzoyl)indolizin-8-yl)-4-chloro-1,2-dimethyl-1H-benzo[d]imidazole-6-carbonitrile and 5-(3-(4-amino-3,5-difluorobenzoyl)indolizin-8-yl)-1,2-dimethyl-1H-benzo[d]imidazole-4,6-dicarbonitrile (61.7 mg).

### Step 2

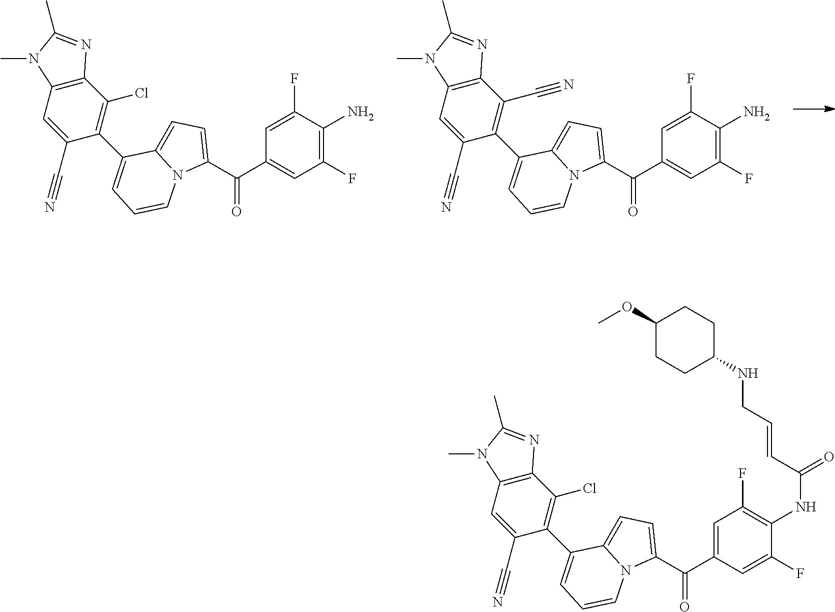 [SMILES: Cc1nc2c(Cl)c(-c3cccn4c(C(=O)c5cc(F)c(N)c(F)c5)ccc34)c(C#N)cc2n1C ; Cc1nc2c(C#N)c(-c3cccn4c(C(=O)c5cc(F)c(N)c(F)c5)ccc34)c(C#N)cc2n1C ; CO[C@H]1CC[C@H](NC/C=C/C(=O)Nc2c(F)cc(C(=O)c3ccc4c(-c5c(C#N)cc6c(nc(C)n6C)c5Cl)cccn34)cc2F)CC1]

A solution of 5-(3-(4-amino-3,5-difluorobenzoyl)indolizin-8-yl)-4-chloro-1,2-dimethyl-1H-benzo[d]imidazole-6-carbonitrile and 5-(3-(4-amino-3,5-difluorobenzoyl)indolizin-8-yl)-1,2-dimethyl-1H-benzo[d]imidazole-4,6-dicarbonitrile (61.7 mg), 4-chlorocrotonic acid (31.3 mg, 0.259 mmol) and propylphosphonic anhydride solution (50 wt % in THF. 243 μL, 0.389 mmol) in DMF (1.00 mL) was stirred at ambient temperature for 20 minutes. Then triethylamine (108 μL, 0.778 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(8-(4-chloro-6-cyano-1,2-dimethyl-1H-benzo[d]imidazol-5-yl)indolizine-3-carbonyl)-2,6-difluorophenyl)but-2-enamide and (E)-4-chloro-N-(4-(8-(4,6-dicyano-1,2-dimethyl-1H-benzo[d]imidazol-5-yl)indolizine-3-carbonyl)-2,6-difluorophenyl)but-2-enamide (70.0 mg, 0.121 mmol), trans-4-methoxycyclohexanamine hydrochloride (40.1 mg, 0.242 mmol), KI (60.3 mg, 0.363 mmol) and K₃PO₄ (103 mg, 0.484 mmol) in DMF (1.00 mL) was stirred at 35° C. for 1 hour. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (8.51 mg, 0.0127 mmol).

### Example 38

### (E)-N-(4-(8-(4-chloro-6-cyclopropyl-1,2-dimethyl-1H-benzo[d]imidazol-5-yl)indolizine-3-carbonyl)-2,6-difluorophenyl)-4-((1r,4r)-4-methoxycyclohexyl)amino)but-2-enamide

### Step 1

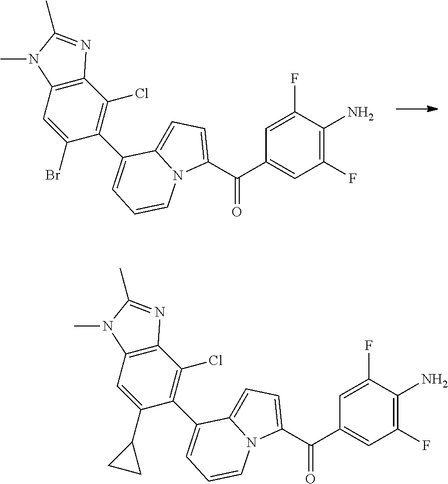 [SMILES: Cc1nc2c(Cl)c(-c3cccn4c(C(=O)c5cc(F)c(N)c(F)c5)ccc34)c(Br)cc2n1C ; Cc1nc2c(Cl)c(-c3cccn4c(C(=O)c5cc(F)c(N)c(F)c5)ccc34)c(C3CC3)cc2n1C]

To a mixture of (4-amino-3,5-difluorophenyl)(8-(6-bromo-4-chloro-1,2-dimethyl-1H-benzo[d]imidazol-5-yl)indolizin-3-yl)methanone (100 mg, 0.189 mmol), cyclopropyl boronic acid (32.5 mg, 0.378 mmol) and Pd(dppf)Cl₂ (30.8 mg, 0.0378 mmol) in 1,2-dimethoxyethane (1.00 mL) was added 2M aqueous sodium carbonate solution (6.82 mL. 13.6 mmol). After reacting by microwave apparatus at 100° C. for 3 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining (4-amino-3,5-difluorophenyl)(8-(4-chloro-6-cyclopropyl-1,2-dimethyl-1H-benzo[d]imidazol-5-yl)indolizin-3-yl)methanone (64.0 mg, 0.130 mmol).

### Step 2

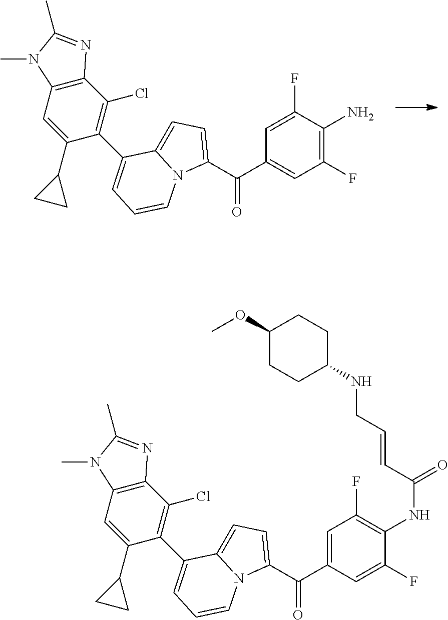 [SMILES: Cc1nc2c(Cl)c(-c3cccn4c(C(=O)c5cc(F)c(N)c(F)c5)ccc34)c(C3CC3)cc2n1C ; CO[C@H]1CC[C@H](NC/C=C/C(=O)Nc2c(F)cc(C(=O)c3ccc4c(-c5c(C6CC6)cc6c(nc(C)n6C)c5Cl)cccn34)cc2F)CC1]

A solution of (4-amino-3,5-difluorophenyl)(8-(4-chloro-6-cyclopropyl-1,2-dimethyl-1H-benzo[d]imidazol-5-yl)indolizin-3-yl)methanone (38.5 mg, 0.0784 mmol), 4-chlorocrotonic acid (18.9 mg, 0.157 mmol) and propylphosphonic anhydride solution (50 wt % in THF. 147 μL, 0.235 mmol) in DMF (1.00 mL) was stirred at ambient temperature for 20 minutes. Then triethylamine (65.3 μL, 0.471 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(8-(4-chloro-6-cyclopropyl-1,2-dimethyl-1H-benzo[d]imidazol-5-yl)indolizine-3-carbonyl)-2,6-difluorophenyl)but-2-enamide (48.1 mg, 0.0784 mmol), trans-4-methoxycyclohexanamine hydrochloride (26.9 mg, 0.162 mmol). KI (40.4 mg, 0.243 mmol) and K₃PO₄ (68.8 mg, 0.324 mmol) in DMF (1.00 mL) was stirred at 35° C. for 1 hour. To the resulting mixture was added saturated aqueous NaHCO₃, and extracted with EtOAc. and washed successively with H₂O then brine, dried over sodium sulfate, and evaporated. The residue was purified by preparative HPLC (water:acetonitrile (0.1% formic acid)) to give the title compound (7.49 mg, 0.0109 mmol).

### Example 39-1 and 39-2

### (E)-N-(2,6-difluoro-4-(8-(4-methoxy-1,2,6-trimethyl-1H-benzo[d]imidazol-5-yl)indolizine-3-carbonyl)phenyl)-4-(((1r,4r)-4-methoxycyclohexyl)amino)but-2-enamide

### Step 1

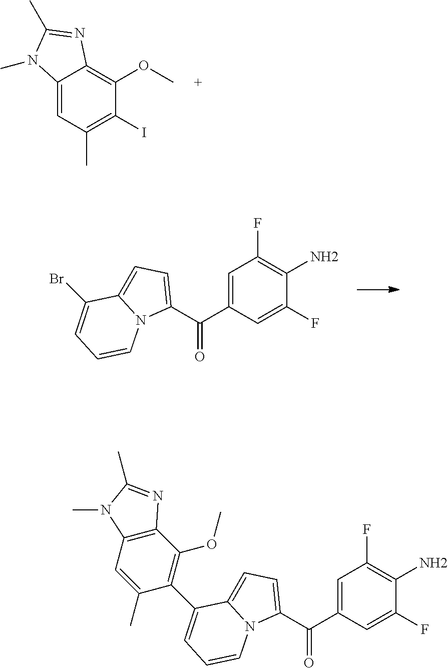

A solution of 5-iodo-4-methoxy-1,2,6-trimethyl-1H-benzo[d]imidazole (230 mg 0.729 mmol). (4-amino-3,5-difluorophenyl)(8-bromoindolizin-3-yl)methanone (336 mg, 0.875 mmol), Pd(PPh3)4 (84 mg, 0.073 mmol) in 1,4-Dioxane (3.7 mL) and 2M Na₂CO₃ aq. (656 μL, 1.31 mmol) was evacuated and N₂ back-filled before heating to 100° C. for 12 hours After cooling, the reaction mixture was diluted with H₂O and CHCl₃. The resulting suspension was filtered, rinsing with CHCl₃, the filtrate was washed with brine then dried over Na₂SO₄ and evaporated. The residue was purified by column chromatography on silica gel (hexane/EtOAc), thereby obtaining (4-amino-3,5-difluorophenyl)(8-(4-methoxy-1,2,6-trimethyl-1H-benzo[d]imidazol-5-yl)indolizin-3-yl)methanone (224 mg, 67%).

### Step 2

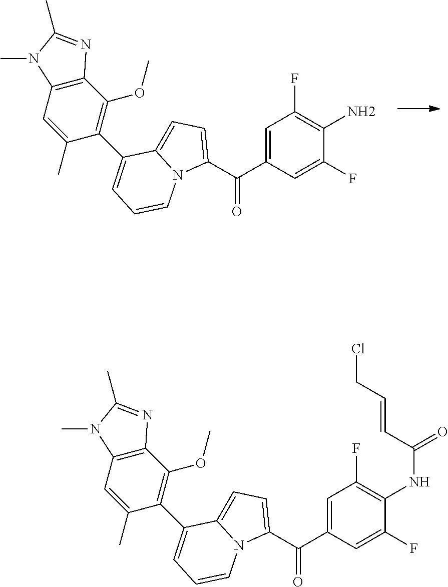 [SMILES: COc1c(-c2cccn3c(C(=O)c4cc(F)c(N)c(F)c4)ccc23)c(C)cc2c1nc(C)n2C ; COc1c(-c2cccn3c(C(=O)c4cc(F)c(NC(=O)/C=C/CCl)c(F)c4)ccc23)c(C)cc2c1nc(C)n2C]

A solution of (4-amino-3,5-difluorophenyl)(8-(4-methoxy-1,2,6-trimethyl-1H-benzo[d]imidazol-5-yl)indolizin-3-yl)methanone (224 mg, 0.486 mmol). 4-chlorocrotonic acid (88 mg, 0.73 mmol) and propylphosphonic anhydride solution (50 wt % in THF. 456 μL, 0.729 mmol) in DMF (1.6 mL) was stirred at RT for 10 min. Then Et₃N (170 μL, 1.22 mmol) was added thereto, and the mixture was stirred at 30° C. for 2 hours Further 4-chlorocrotonic acid (15 mg, 0.12 mmol), propylphosphonic anhydride solution (50 wt % in THF, 76.0 μL, 0.122 mmol) and Et₃N (34.0 μL, 0.243 mmol) were added and stirring continued for 1 hour The reaction mixture was diluted with H₂O and sat. NaHCO₃ aq. then stirred at RT for 1 hour The resulting solid was collected by filtration and vacuumed at RT, thereby obtaining crude (E)-4-chloro-N-(2,6-difluoro-4-(8-(4-methoxy-1,2,6-trimethyl-1H-benzo[d]imidazol-5-yl)indolizine-3-carbonyl)phenyl)but-2-enamide (235 mg, 86%).

### Step 3

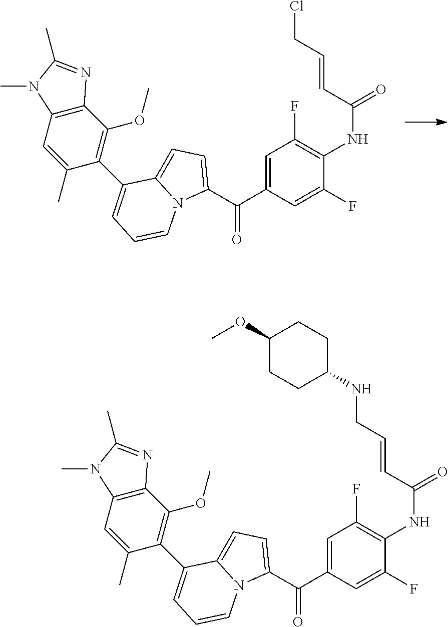 [SMILES: COc1c(-c2cccn3c(C(=O)c4cc(F)c(NC(=O)/C=C/CCl)c(F)c4)ccc23)c(C)cc2c1nc(C)n2C ; COc1c(-c2cccn3c(C(=O)c4cc(F)c(NC(=O)/C=C/CN[C@H]5CC[C@H](OC)CC5)c(F)c4)ccc23)c(C)cc2c1nc(C)n2C]

A solution of crude (E)-4-chloro-N-(2,6-difluoro-4-(8-(4-methoxy-1,2,6-trimethyl-1H-benzo[d]imidazol-5-yl)indolizine-3-carbonyl)phenyl)but-2-enamide (129 mg), trans-4-methoxycyclohexanamine hydrochloride (114 mg, 0.687 mmol), KI (114 mg, 0.687 mmol) and K₃PO₄ (292 mg, 1.37 mmol) in DMF (763 μL) was stirred at 30° C. for 2 hours The reaction mixture was diluted with H₂O then stirred at RT for 3 hours The resulting solid was collected by filtration and vacuum-dried at RT. The obtained solid was purified by column chromatography on silica gel (CHCl₃ (1% Et₃N)/MeOH) and preparative HPLC (water:acetonitrile (0.1% formic acid)). The purified fractions were washed with sat. NaHCO₃ aq., extracted with CHCl₃/MeOH then dried over Na₂SO₄ and evaporated, thereby obtaining the racemic title compound (70 mg). The racemate was separated by CHIRALPAK IE (hexane-EtOH(0.1% Et₃N)) to give the title compound (Example 39-1, 28.2 mg, 19%; single isomer. 1st-eluting isomer and Example 39-2, 27.7 mg, 19%; single isomer, 2nd-eluting isomer).

### Example 40-1 and 40-2

### (E)-4-(tert-butylamino)-N-(2,6-difluoro-4(8-(4-methoxy-1,2,6-trimethyl-1H-benzo[d]imidazol-5-yl)indolizine-3-carbonyl)phenyl but-2-enamide

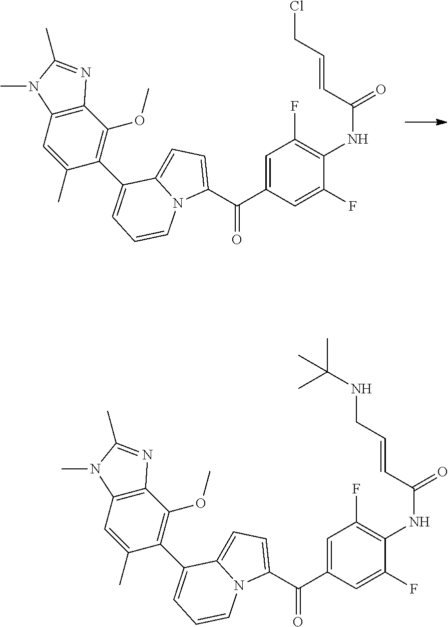 [SMILES: COc1c(-c2cccn3c(C(=O)c4cc(F)c(NC(=O)/C=C/CCl)c(F)c4)ccc23)c(C)cc2c1nc(C)n2C ; COc1c(-c2cccn3c(C(=O)c4cc(F)c(NC(=O)/C=C/CNC(C)(C)C)c(F)c4)ccc23)c(C)cc2c1nc(C)n2C]

A solution of crude (E)-4-chloro-N-(2,6-difluoro-4-(8-(4-methoxy-1,2,6-trimethyl-1H-benzo[d]imidazol-5-yl)indolizine-3-carbonyl)phenyl)but-2-enamide (106 mg), tert-Butylamine (148 μL, 1.41 mmol), KI (94 mg, 0.57 mmol) in DMF (627 μL) was stirred at 30° C. for 2 hours The reaction mixture was purified in a similar manner to Example 39-1, 39-2. The racemate (68 mg) was separated by CHIRALPAK IE (hexane-EtOH(0.1% Et₃N)) to give the title compound (Example 40-1, 26.4 mg, 23%; single isomer. 1st-eluting isomer and Example 40-2, 26.4 mg, 23%; single isomer, 2nd-eluting isomer).

### Example 41-1 and 41-2

### (E)-N-(4-(8-(4-chloro-6-methoxy-1-methyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

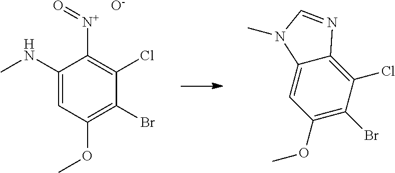

The mixture of 4-bromo-3-chloro-5-methoxy-N-methyl-2-nitroaniline (410 mg. 1.39 mmol) in formic acid (8.20 mL, 217 mmol) was stirred at 100° for 1 hour. Iron (542 mg, 9.71 mmol) was added thereto, and the mixture was stirred at 85° C. for 1 hour. After cooling, the resulting mixture was diluted with EtOAc and saturated aqueous NaHCO3, and the insolubles were removed by filtration. The organic layer was separated, washed with brine, dried over sodium sulfate, and evaporated. The residue was purified by column chromatography on silica gel (EtOAc-hexane) to give 5-bromo-4-chloro-6-methoxy-1-methyl-1H-benzo[d]imidazole (49.0 mg, 0.180 mmol).

### Step 2

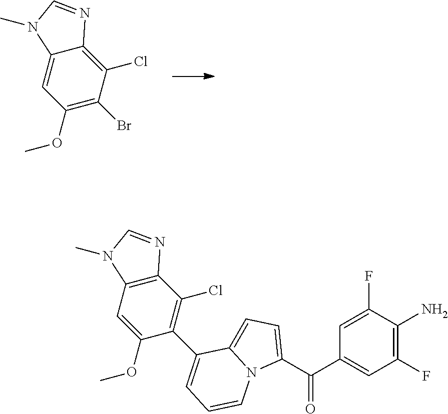 [SMILES: COc1cc2c(ncn2C)c(Cl)c1Br ; COc1cc2c(ncn2C)c(Cl)c1-c1cccn2c(C(=O)c3cc(F)c(N)c(F)c3)ccc12]

To a mixture of 5-bromo-4-chloro-6-methoxy-1-methyl-1H-benzo[d]imidazole (83.0 mg, 0.301 mmol), (4-amino-3,5-difluorophenyl)(8-(5,5-dimethyl-1,3,2-dioxaborinan-2-yl)indolizin-3-yl)methanone (139 mg, 0.361 mmol) and Pd(PPh3)4 (34.8 mg, 0.0301 mmol) in 1,2-dimethoxyethane (4.80 mL) was added 2M aqueous sodium carbonate solution (301 μL, 0.602 mmol). After reacting by microwave apparatus at 120° C. for 3 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining (4-amino-3,5-difluorophenyl)(8-(4-chloro-6-methoxy-1-methyl-1H-benzo[d]imidazol-5-yl)indolizin-3-yl)methanone (140 mg, 0.300 mmol).

### Step 3

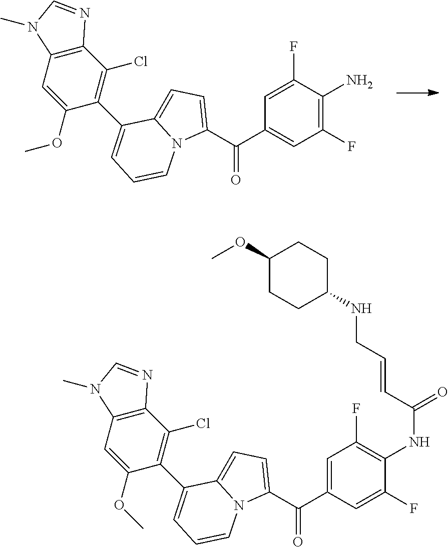 [SMILES: COc1cc2c(ncn2C)c(Cl)c1-c1cccn2c(C(=O)c3cc(F)c(N)c(F)c3)ccc12 ; COc1cc2c(ncn2C)c(Cl)c1-c1cccn2c(C(=O)c3cc(F)c(NC(=O)/C=C/CN[C@H]4CC[C@H](OC)CC4)c(F)c3)ccc12]

A solution of (4-amino-3,5-difluorophenyl)(8-(4-chloro-6-methoxy-1-methyl-1H-benzo[d]imidazol-5-yl)indolizin-3-yl)methanone (140 mg, 0.300 mmol), 4-chlorocrotonic acid (72.3 mg, 0.600 mmol) and propylphosphonic anhydride solution (50 wt % in THF. 562 μL, 0.900 mmol) in DMF (4.30 mL) was stirred at ambient temperature for 20 minutes. Then triethylamine (250 μL, 1.80 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(8-(4-chloro-6-methoxy-1-methyl-1H-benzo[d]imidazol-5-yl)indolizine-3-carbonyl)-2,6-difluorophenyl)but-2-enamide (171 mg, 0.300 mmol), trans-4-methoxycyclohexanamine hydrochloride (99.5 mg, 0.6100 mmol), KI (150 mg, 0.900 mmol) and K₃PO₄ (255 mg, 1.20 mmol) in DMF (2.00 mL) was stirred at at 35° C. for 1 hour. To the resulting mixture was added saturated aqueous NaHCO₃, and extracted with EtOAc, and washed successively with H₂O then brine, dried over sodium sulfate, and evaporated. The residue was purified by column chromatography on silica gel (methanol-EtOAc-hexane), thereby obtaining the racemate title compound (83.2 mg, 0.126 mmol). The racemate was separated by CHIRALPAK IE (hexane-ethanol(0.1% triethyl amine)) to give the title compound (20.8 mg, single isomer. 1^{st}-eluting isomer) and (19.1 mg, single isomer. 2^{nd}-eluting isomer).

### Example 42

### (E)-N-(4-(8-(2-chloro-6-methoxy-4-(1-methyl-1H-imidazol-5-yl)phenyl)indolizine-3-carbonyl)-2,6-difluorophenyl)-4-(((1r,4r)-4-methoxycyclohexyl)amino)but-2-enamide

### Step 1

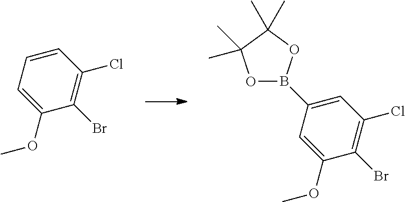

A solution of 2-bromo-1-chloro-3-methoxybenzene (1.5 g), Bis(pinacolato)diboron (2.6 g). (1,5-Cyclooctadiene)(methoxy)iridium(I) Dimer (89.8 mg), 4,4′-Di-tert-butyl-2,2′-bipyridine (72.7 mg) in heptane (15.0 mL) was stirred at 80° C. for 2 hours. After evaporation, the obtained residue was purified by column chromatography (hexane:ethyl acetate), thereby obtaining 2-(4-bromo-3-chloro-5-methoxyphenyl)-4,4,5,5-tetramethyl-1,3,2-dioxaborolane (1.7 g).

### Step 2

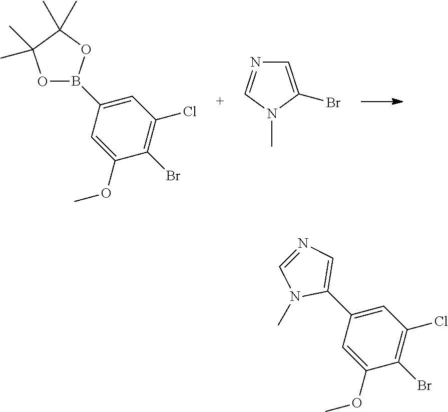

A solution of 2-(4-bromo-3-chloro-5-methoxyphenyl)-4,4,5,5-tetramethyl-1,3,2-dioxaborolane (80.0 mg) obtained in step 1, 5-bromo-1-methyl-1H-imidazole (37.1 mg), tetrakis(triphenylphosphine)palladium(0) (26.6 mg), 2.0 M sodium bicarbonate in water (265 μL) in 1,2-dimethoxyethane (1.2 mL) was stirred at 120° C. for 2 hours using microwave irradiation. After dilution with ethyl acetate, the mixture was extracted with ethyl acetate. The combined organic layer was washed with water and a saturated sodium chloride solution, followed by drying over sodium sulfate. After evaporation, the obtained residue was purified by column chromatography (hexane:ethyl acetate), thereby obtaining 5-(4-bromo-3-chloro-5-methoxyphenyl)-1-methyl-1H-imidazole (50.0 mg).

### Step 3-5

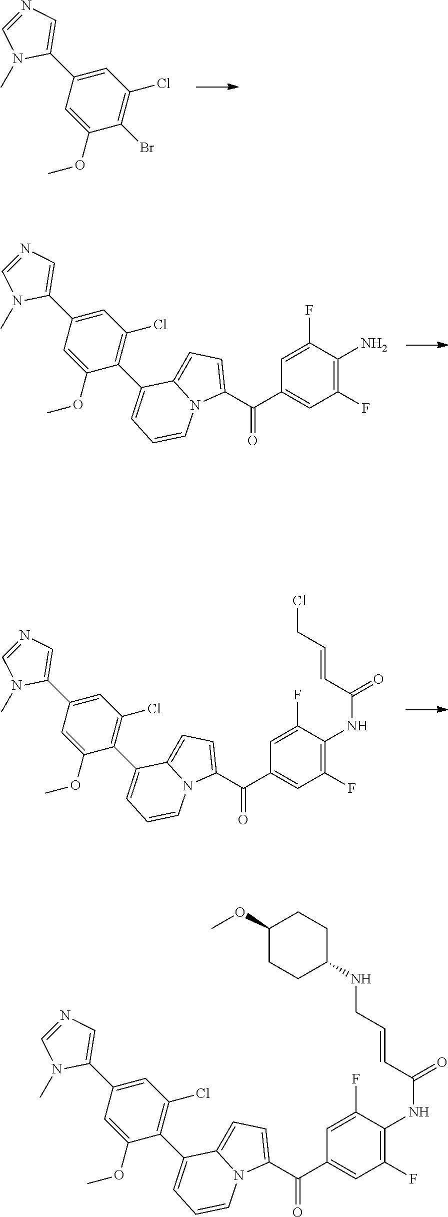 [SMILES: COc1cc(-c2cncn2C)cc(Cl)c1Br ; COc1cc(-c2cncn2C)cc(Cl)c1-c1cccn2c(C(=O)c3cc(F)c(N)c(F)c3)ccc12 ; COc1cc(-c2cncn2C)cc(Cl)c1-c1cccn2c(C(=O)c3cc(F)c(NC(=O)/C=C/CCl)c(F)c3)ccc12 ; COc1cc(-c2cncn2C)cc(Cl)c1-c1cccn2c(C(=O)c3cc(F)c(NC(=O)/C=C/CN[C@H]4CC[C@H](OC)CC4)c(F)c3)ccc12]

The procedures of Example 10 (steps 6-8) were performed except that 5-(4-bromo-3-chloro-5-methoxyphenyl)-1-methyl-1H-imidazole (50.0 mg) obtained in step 2 was used instead of 5-bromo-4-chloro-6-ethyl-1-methyl-1H-benzo[d]imidazole used in Example 10 (step 6), thereby obtaining title compound (5.28 mg).

### Example 43

### (E)-N-(4-(8-(4-chloro-6-ethynyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

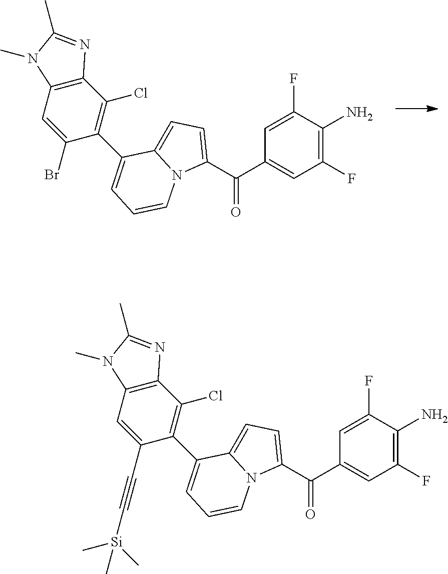 [SMILES: Cc1nc2c(Cl)c(-c3cccn4c(C(=O)c5cc(F)c(N)c(F)c5)ccc34)c(Br)cc2n1C ; Cc1nc2c(Cl)c(-c3cccn4c(C(=O)c5cc(F)c(N)c(F)c5)ccc34)c(C#C[Si](C)(C)C)cc2n1C]

To a mixture of (4-amino-3,5-difluorophenyl)(8-(6-bromo-4-chloro-1,2-dimethyl-1H-benzo[d]imidazol-5-yl)indolizin-3-yl)methanone (100 mg, 0.189 mmol), trimethylsilylacetylene (80.0 μL, 0.566 mmol), CuI (3.60 mg, 0.0189 mmol) and Pd(dppf)Cl₂ (15.4 mg, 0.0189 mmol) in DMF (1.00 mL) was added triethylamine (158 μL. 0.556 mmol). After reacting by microwave apparatus at 120° C. for 2 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining (4-amino-3,5-difluorophenyl)(8-(4-chloro-1,2-dimethyl-6-((trimethylsilyl)ethynyl)-1H-benzo[d]imidazol-5-yl)indolizin-3-yl)methanoneyl)indolizin-3-yl)methanone (56.1 mg, 0.103 mmol).

### Step 2

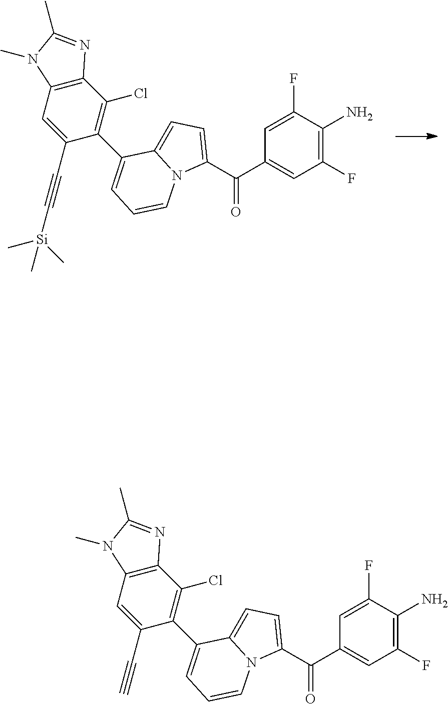 [SMILES: Cc1nc2c(Cl)c(-c3cccn4c(C(=O)c5cc(F)c(N)c(F)c5)ccc34)c(C#C[Si](C)(C)C)cc2n1C ; C#Cc1cc2c(nc(C)n2C)c(Cl)c1-c1cccn2c(C(=O)c3cc(F)c(N)c(F)c3)ccc12]

Tetrabutylammonium fluoride (IM THF solution, 208 μl, 0.208 mmol) was added to a solution of (4-amino-3,5-difluorophenyl)(8-(4-chloro-1,2-dimethyl-6-((trimethylsilyl)ethynyl)-1H-benzo[d]imidazol-5-yl)indolizin-3-yl)methanoneyl)indolizin-3-yl)methanone (57.0 mg, 0.104 mmol) in THE (1.00 mL). After the mixture was stirred at ambient temperature for 20 minutes, the mixture was evaporated. The residue was purified by column chromatography on silica gel (EtOAc-hexane), thereby obtaining (4-amino-3,5-difluorophenyl)(8-(4-chloro-6-ethynyl-1,2-dimethyl-1H-benzo[d]imidazol-5-yl)indolizin-3-yl)methanone (38.5 mg, 0.0811 mmol).

### Step 3

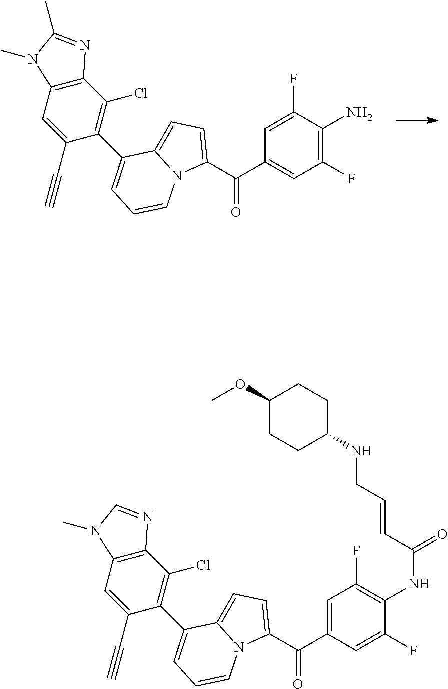 [SMILES: C#Cc1cc2c(nc(C)n2C)c(Cl)c1-c1cccn2c(C(=O)c3cc(F)c(N)c(F)c3)ccc12 ; C#Cc1cc2c(ncn2C)c(Cl)c1-c1cccn2c(C(=O)c3cc(F)c(NC(=O)/C=C/CN[C@H]4CC[C@H](OC)CC4)c(F)c3)ccc12]

A solution of (4-amino-3,5-difluorophenyl)(8-(4-chloro-6-ethynyl-1,2-dimethyl-1H-benzo[d]imidazol-5-yl)indolizin-3-yl)methanone (42.8 mg, 0.0901 mmol), 4-chlorocrotonic acid (21.7 mg, 0.180 mmol) and propylphosphonic anhydride solution (50 wt % in THF. 169 μL, 0.270 mmol) in DMF (1.00 mL) was stirred at ambient temperature for 20 minutes. Then triethylamine (75.1 μL, 0.541 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(8-(4-chloro-6-ethynyl-1,2-dimethyl-1H-benzo[d]imidazol-5-yl)indolizine-3-carbonyl)-2,6-difluorophenyl)but-2-enamide (52.0 mg, 0.0901 mmol), trans-4-methoxycyclohexanamine hydrochloride (29.8 mg, 0.180 mmol), KI (44.8 mg, 0.270 mmol) and K₃PO4 (76.5 mg, 0.360 mmol) in DMF (1.00 mL) was stirred at 35° C. for 1 hour. To the resulting mixture was added saturated aqueous NaHCO₃, and extracted with EtOAc. and washed successively with H₂O then brine, dried over sodium sulfate, and evaporated. The residue was purified by preparative HPLC (water:acetonitrile (0.1% formic acid)) to give the title compound (11.6 mg, 0.0173 mmol).

### Example 44

### (E)-N-(2,6-difluoro-4-(8-(4-fluoro-6-methoxy-1,2-dimethyl-1H-benzo[d]imidazol-5-yl)indolizine-3-carbonyl)phenyl-4-(((1r,4r)-4-methoxycyclohexyl)amino)but-2-enamide

The title compound was obtained using the same procedure as that of other examples.

### Example 45

### (E)-N-(4-(7-(1,2-dimethyl-6-(trifluoromethyl)-1H-benzo[d]imidazol-5-yl)-1H-indole-3-carbonyl)-2,6-difluorophenyl)-4-(((1r,4r)-4-methoxycyclohexyl)amino)but-2-enamide

### Step 1

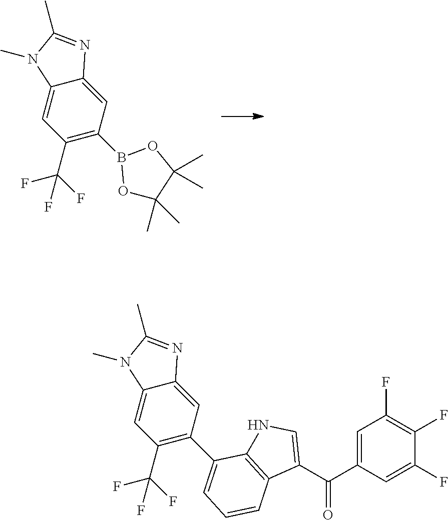

To a mixture of 1,2-dimethyl-5-(4,4,5,5-tetramethyl-1,3,2-dioxaborolan-2-yl)-6-(trifluoromethyl)benzimidazole (420 mg, 0.988 mmol), (7-bromo-1H-indol-3-yl)(3,4,5-trifluorophenyl)methanone (250 mg, 0.713 mmol) and Pd(PPh₃)₄ (81.6 mg, 0.0713 mmol) in 1,2-dimethoxyethane (2.00 mL) was added 2M aqueous sodium carbonate solution (700 μL, 1.41 mmol). After reacting by microwave apparatus at 120° C. for 2 hours, the mixture was diluted with EtOAc and water. The organic layer was washed successively with H₂O then brine, dried over sodium sulfate, and evaporated. The residue was purified by column chromatography on silica gel (acetone-hexane), thereby obtaining (7-(1,2-dimethyl-6-(trifluoromethyl)-1H-benzo[d]imidazol-5-yl)-1H-indol-3-yl)(3,4,5-trifluorophenyl)methanone (490 mg, 1.01 mmol).

### Step 2

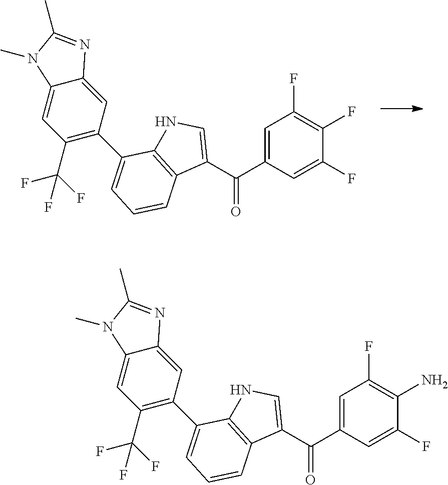 [SMILES: Cc1nc2cc(-c3cccc4c(C(=O)c5cc(F)c(F)c(F)c5)c[nH]c34)c(C(F)(F)F)cc2n1C ; Cc1nc2cc(-c3cccc4c(C(=O)c5cc(F)c(N)c(F)c5)c[nH]c34)c(C(F)(F)F)cc2n1C]

To a mixture of (7-(1,2-dimethyl-6-(trifluoromethyl)-1H-benzo[d]imidazol-5-yl)-1H-indol-3-yl)(3,4,5-trifluorophenyl)methanone (344 mg, 0.711 mmol) in 1,4-dioxane (3.4 mL) was added ammonium hydroxide (14 mL, 212 mmol). After reacting by microwave apparatus at 130° C. for 14 hours, the mixture was diluted with EtOAc and water. The organic layer was washed successively with H₂O then brine, dried over sodium sulfate, and evaporated. The residue was purified by column chromatography on silica gel (acetone-hexane), thereby obtaining (4-amino-3,5-difluorophenyl)(7-(1,2-dimethyl-6-(trifluoromethyl)-1H-benzo[d]imidazol-5-yl)-1H-indol-3-yl)methanone (167 mg, 0.340 mmol).

### Step 3

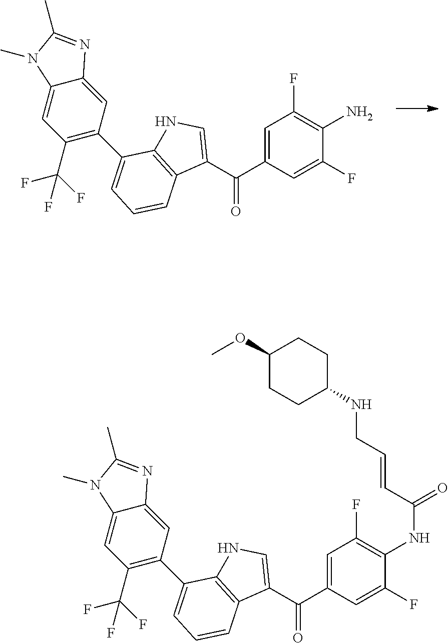 [SMILES: Cc1nc2cc(-c3cccc4c(C(=O)c5cc(F)c(N)c(F)c5)c[nH]c34)c(C(F)(F)F)cc2n1C ; CO[C@H]1CC[C@H](NC/C=C/C(=O)Nc2c(F)cc(C(=O)c3c[nH]c4c(-c5cc6nc(C)n(C)c6cc5C(F)(F)F)cccc34)cc2F)CC1]

A solution of (4-amino-3,5-difluorophenyl)(7-(1,2-dimethyl-6-(trifluoromethyl)-1H-benzo[d]imidazol-5-yl)-1H-indol-3-yl)methanone (167 mg, 0.352 mmol), 4-chlorocrotonic acid (83.1 mg, 0.692 mmol) and propylphosphonic anhydride solution (50 wt % in THF, 646 μL, 1.03 mmol) in DMF (3.00 mL) was stirred at ambient temperature for 20 minutes. Then triethylamine (287 μL, 2.07 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(7-(1,2-dimethyl-6-(trifluoromethyl)-1H-benzo[d]imidazol-5-yl)-1H-indole-3-carbonyl)-2,6-difluorophenyl)but-2-enamide (202 mg, 0.352 mmol), trans-4-methoxycyclohexanamine hydrochloride (114 mg, 0.683 mmol). KI (171 mg, 1.03 mmol) and K₃PO₄ (292 mg, 1.38 mmol) in DMF (4.00 mL) was stirred at 35° C. for 1 hour. To the resulting mixture was added saturated aqueous NaHCO₃, and extracted with EtOAc. and washed successively with H₂O then brine, dried over sodium sulfate, and evaporated. The residue was purified by preparative HPLC (water:acetonitrile (0.1% formic acid)) to give the title compound (48.8 mg, 0.0721 mmol).

### Example 46

### (E)-4-(tert-butylamino)-N-(2,6-difluoro-4-(8-(4-fluoro-6-methoxy-1,2-dimethyl-1H-benzo[d]imidazol-5-yl)indolizine-3-carbonyl)phenyl)but-2-enamide

The title compound was obtained using the same procedure as that of other examples.

### Example 47, 48

### (E)-N-(4-(8-(4-chloro-2-((R)-1-methoxyethyl)-1,6-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

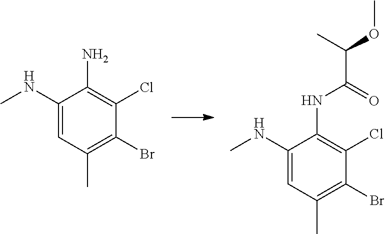

A solution of 4-bromo-3-chloro-N1,5-dimethylbenzene-1,2-diamine (200 mg), (R)-2-methoxypropanoic acid (0.0834 mL, 0.882 mmol), 1-hydroxybenzotriazole hydrate (153 mg, 1.00 mmol), 1-(3-Dimethylaminopropyl)-3-ethylcarbodiimide hydrochloride (192 mg, 1.00 mol), N,N-Diisopropylethylamine (0.341 mL, 2.00 mmol) in DMF (2 mL) was stirred at RT overnight. (R)-2-methoxypropanoic acid (0.0985 mL, 1.04 mmol) and propylphosphonic anhydride solution (50 wt % in THF, 1.25 mL, 2.00 mmol) were added and the mixture was stirred at RT for 20 min. Then triethylamine (0.445 mL, 3.21 mmol) was added thereto, and the mixture was stirred at RT overnight. To the resulting mixture was added EtOAc, saturated aqueous NaHCO₃, and water, and the organic layer was washed successively with H₂O, saturated aqueous NaHCO₃ then brine, dried over sodium sulfate, and evaporated to give crude (R)—N-(3-bromo-2-chloro-4-methyl-6-(methylamino)phenyl)-2-methoxypropanamide (266 mg).

### Step 2

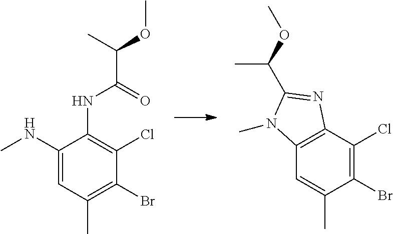

A solution of crude (R)—N-(3-bromo-2-chloro-4-methyl-6-(methylamino)phenyl)-2-methoxypropanamide (266 mg) in AcOH (6.82 mL) was stirred at RT overnight. The mixture was evaporated and the residue was diluted with H₂O and saturated aqueous NaHCO₃, and extracted with EtOAc. The organic layer was washed with H₂O, saturated aqueous NaHCO₃, and brine, dried over sodium sulfate, and evaporated. The residue was purified by column chromatography on silica gel (Hexane-EtOAc (10% CHCl₃)), thereby obtaining (R)-5-bromo-4-chloro-2-(1-methoxyethyl)-1,6-dimethyl-1H-benzo[d]imidazole (202 mg).

### Step 3

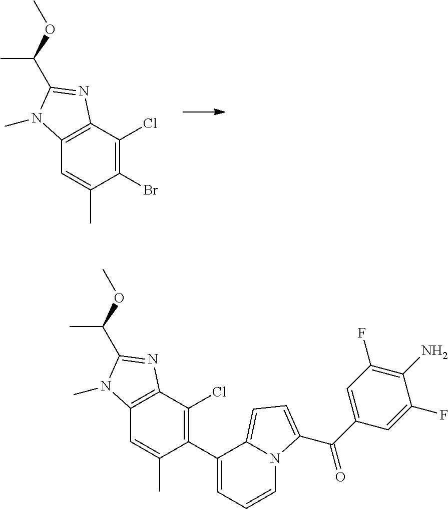 [SMILES: CO[C@H](C)c1nc2c(Cl)c(Br)c(C)cc2n1C ; CO[C@H](C)c1nc2c(Cl)c(-c3cccn4c(C(=O)c5cc(F)c(N)c(F)c5)ccc34)c(C)cc2n1C]

The procedure of Example 18 (Step 2) was performed except that (R)-5-bromo-4-chloro-2-(1-methoxyethyl)-1,6-dimethyl-1H-benzo[d]imidazole (100 mg) was used instead of 5-bromo-4-chloro-1,2,6-trimethyl-1H-benzo[d]imidazole in Example 18 (Step 2), thereby obtaining crude (R)-(4-amino-3,5-difluorophenyl)(8-(4-chloro-2-(1-methoxyethyl)-1,6-dimethyl-1H-benzo[d]imidazol-5-yl)indolizin-3-yl)methanone (210 mg).

### Step 4

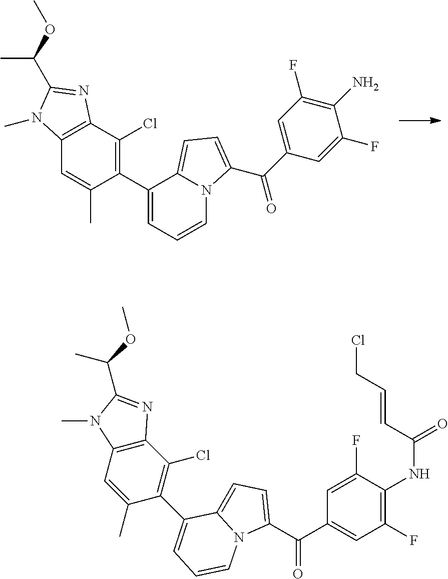 [SMILES: CO[C@H](C)c1nc2c(Cl)c(-c3cccn4c(C(=O)c5cc(F)c(N)c(F)c5)ccc34)c(C)cc2n1C ; CO[C@H](C)c1nc2c(Cl)c(-c3cccn4c(C(=O)c5cc(F)c(NC(=O)/C=C/CCl)c(F)c5)ccc34)c(C)cc2n1C]

The procedure of Example 2 (step 2) was performed except that crude (R)-(4-amino-3,5-difluorophenyl)(8-(4-chloro-2-(1-methoxyethyl)-1,6-dimethyl-1H-benzo[d]imidazol-5-yl)indolizin-3-yl)methanone (210 mg) obtained in step 3 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2), thereby obtaining crude (R,E)-4-chloro-N-(4-(8-(4-chloro-2-(1-methoxyethyl)-1,6-dimethyl-1H-benzo[d]imidazol-5-yl)indolizine-3-carbonyl)-2,6-difluorophenyl)but-2-enamide (234 mg).

### Step 5

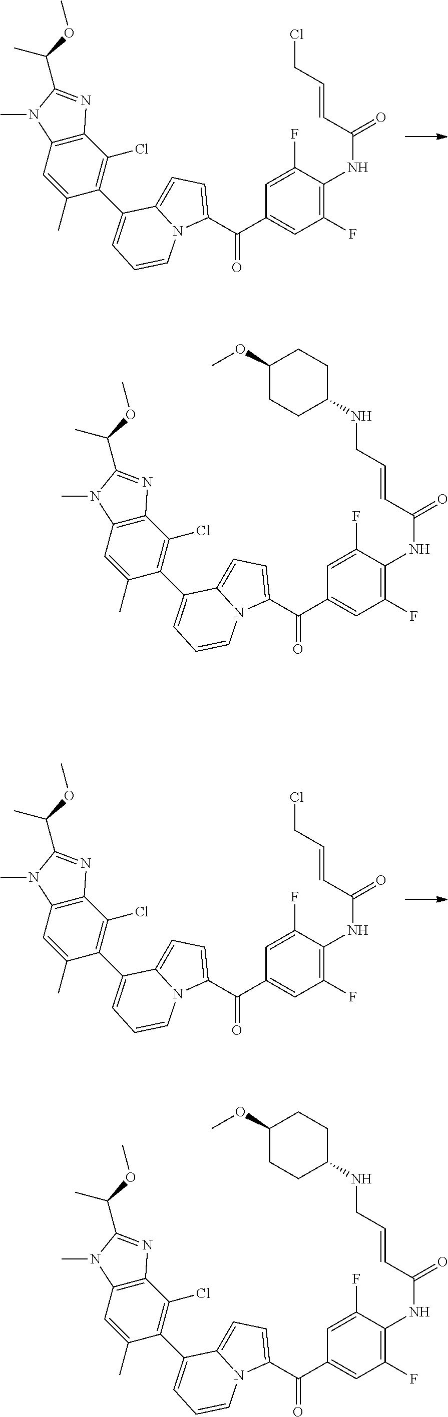 [SMILES: CO[C@H](C)c1nc2c(Cl)c(-c3cccn4c(C(=O)c5cc(F)c(NC(=O)/C=C/CCl)c(F)c5)ccc34)c(C)cc2n1C ; CO[C@H](C)c1nc2c(Cl)c(-c3cccn4c(C(=O)c5cc(F)c(NC(=O)/C=C/CN[C@H]6CC[C@H](OC)CC6)c(F)c5)ccc34)c(C)cc2n1C ; CO[C@H](C)c1nc2c(Cl)c(-c3cccn4c(C(=O)c5cc(F)c(NC(=O)/C=C/CCl)c(F)c5)ccc34)c(C)cc2n1C ; CO[C@H](C)c1nc2c(Cl)c(-c3cccn4c(C(=O)c5cc(F)c(NC(=O)/C=C/CN[C@H]6CC[C@H](OC)CC6)c(F)c5)ccc34)c(C)cc2n1C]

The procedure of Example 2 (step 3) was performed except that crude (R,E)-4-chloro-N-(4-(8-(4-chloro-2-(1-methoxyethyl)-1,6-dimethyl-1H-benzo[d]imidazol-5-yl)indolizine-3-carbonyl)-2,6-difluorophenyl)but-2-enamide (117 mg) obtained in step 4 was used instead of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide in Example 2 (step 3), thereby obtaining the title diastereomeric compound (52.3 mg). The diastereomer was separated by CHIRAL ART SB (hexane-ethanol (0.1% triethyl amine)) to give the title compound (Example 47, 17.4 mg; single isomer, 1^{st}-eluting isomer and Example 48, 17.1 mg; single isomer. 2^{nd}-eluting isomer).

### Example 49, 50

### (R,E)-4-(tert-butylamino)-N-(4-(8-(4-chloro-2-(1-methoxyethyl)-1,6-dimethyl-1H-benzo[d]imidazol-5-yl)indolizine-3-carbonyl)-2,6-difluorophenyl)but-2-enamide

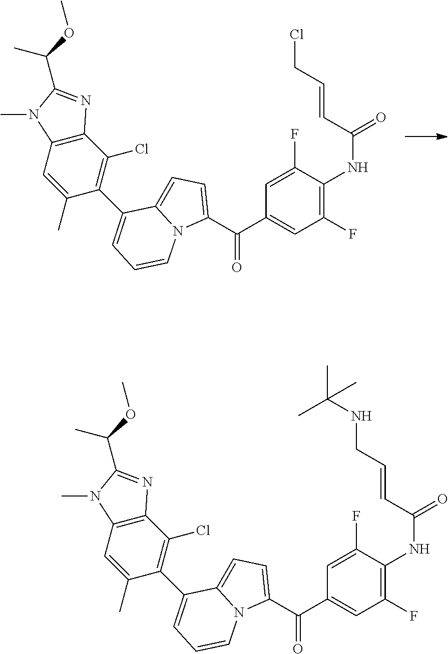 [SMILES: CO[C@H](C)c1nc2c(Cl)c(-c3cccn4c(C(=O)c5cc(F)c(NC(=O)/C=C/CCl)c(F)c5)ccc34)c(C)cc2n1C ; CO[C@H](C)c1nc2c(Cl)c(-c3cccn4c(C(=O)c5cc(F)c(NC(=O)/C=C/CNC(C)(C)C)c(F)c5)ccc34)c(C)cc2n1C]

The procedure of Example 18 (step 4) was performed except that crude (R,E)-4-chloro-N-(4-(8-(4-chloro-2-(I-methoxyethyl)-1,6-dimethyl-1H-benzo[d]imidazol-5-yl)indolizine-3-carbonyl)-2,6-difluorophenyl)but-2-enamide (117 mg) was used instead of (E)-4-chloro-N-(4-(8-(4-chloro-1,2,6-trimethyl-1H-benzo[d]imidazol-5-yl)indolizine-3-carbonyl)-2,6-difluorophenyl)but-2-enamide in Example 18 (step 4), thereby obtaining the diastereomic title compound (41.7 mg). The diastereomer was separated by CHIRAL ART SB (hexane-ethanol (0.1% triethyl amine)) to give the title compound (Example 49, 14.4 mg; single isomer, 1^{st}-eluting isomer and Example 50, 14.9 mg; single isomer. 2^{nd}-eluting isomer).

### Example 51

### (E)-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)-4-(((1r,4r)-4-methoxycyclohexyl)amino)but-2-enamide

### Step 1

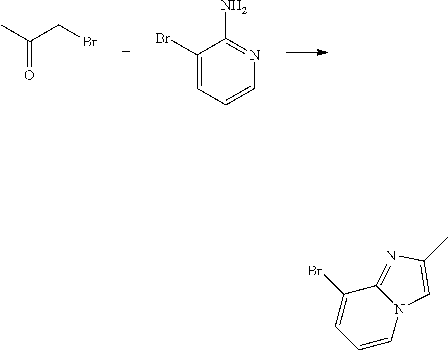

A solution of 1-bromopropan-2-one (840 μL) and 3-bromopyridin-2-amine (1.73 g) in ethanol (10 mL) was stirred at 70° C. for 48 hours. The solvent was evaporated under reduced pressure. After dilution with ethyl acetate, the mixture was washed with a saturated sodium bicarbonate solution and a saturated sodium chloride solution, followed by drying over sodium sulfate. The solvent was evaporated under reduced pressure, thereby obtaining crude 8-bromo-2-methylimidazo[1,2-a]pyridine(2.10 g).

### Step 2

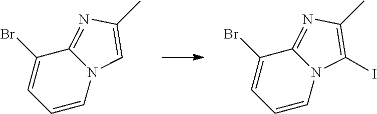

A solution of crude 8-bromo-2-methylimidazo[1,2-a]pyridine (2.10 g) obtained in step 1 and N-Iodosuccinimide (2.50 g) in acetonitrile (20 mL) was stirred at 30° C. for 1 hour. After the solvent was reduced by half under reduced pressure, the precipitate was collected by filtration. After washing with acetonitrile, the residue was dried at 50° C. under reduced pressure, thereby obtaining crude 8-bromo-3-iodo-2-methylimidazo[1,2-a]pyridine (2.59 g).

### Step 3

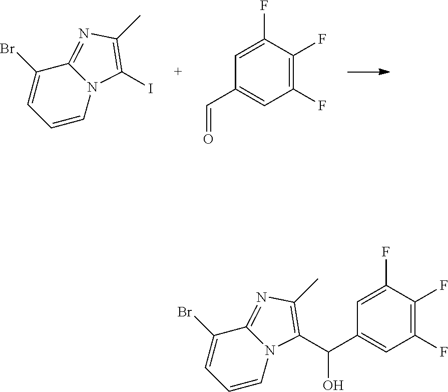 [SMILES: Cc1nc2c(Br)cccn2c1I ; O=Cc1cc(F)c(F)c(F)c1 ; Cc1nc2c(Br)cccn2c1C(O)c1cc(F)c(F)c(F)c1]

A solution of crude 8-bromo-3-iodo-2-methylimidazo[1,2-a]pyridine (1.91 g) obtained in step 2 in tetrahydrofuran (20 mL) was cooled to −10° C. To the reaction mixture was added isopropylmagnesium chloride-lithium chloride complex (tetrahydrofuran solution (abt. 14%), 8.72 mL). After stirring at −10° C. for 1 hour. 3,4,5-trifluorobenzaldehyde (1.82 g) was added to the reaction mixture. A reaction was performed at −10° C. for 1 hour. After warming up to room temperature, the reaction mixture was quenched with a saturated ammonium chloride solution. After the mixture was extracted with ethyl acetate, the organic layer was washed with a saturated sodium chloride solution, followed by drying over sodium sulfate. After evaporation, the obtained residue was purified by column chromatography (hexane:ethyl acetate), thereby obtaining (8-bromo-2-methylimidazo[1,2-a]pyridin-3-yl)(3,4,5-trifluorophenyl)methanol (1.50 g).

### Step 4

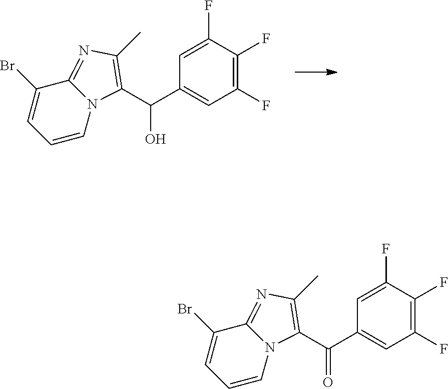 [SMILES: Cc1nc2c(Br)cccn2c1C(O)c1cc(F)c(F)c(F)c1 ; Cc1nc2c(Br)cccn2c1C(=O)c1cc(F)c(F)c(F)c1]

A solution of (8-bromo-2-methylimidazo[1,2-a]pyridin-3-yl)(3,4,5-trifluorophenyl)methanol (1.50 g) obtained in step 3 and manganese dioxide (1.78 g) in ethyl acetate (30 mL) was stirred at 60° C. for 14 hours. The insoluble matter was filtered off, followed by evaporating the solvent under reduced pressure. The obtained residue was purified by column chromatography (hexane:ethyl acetate), thereby obtaining (8-bromo-2-methylimidazo[1,2-a]pyridin-3-yl)(3,4,5-trifluorophenyl)methanone (1.48 g).

### Step 5

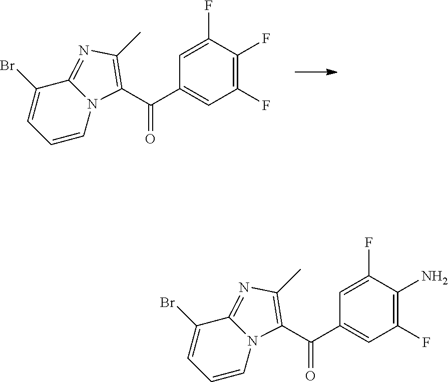 [SMILES: Cc1nc2c(Br)cccn2c1C(=O)c1cc(F)c(F)c(F)c1 ; Cc1nc2c(Br)cccn2c1C(=O)c1cc(F)c(N)c(F)c1]

A solution of (8-bromo-2-methylimidazo[1,2-a]pyridin-3-yl)(3,4,5-trifluorophenyl)methanone (1.48 g) obtained in step 4 and 28% ammonia solution (6 mL) in 1,4-dioxane (10 mL) was stirred at 140° C. for 8 hours using microwave irradiation. After dilution with ethyl acetate and water, the mixture was extracted with ethyl acetate. The combined organic layer was washed with a saturated sodium chloride solution, followed by drying over sodium sulfate. After evaporation, the obtained residue was purified by column chromatography (hexane:ethyl acetate), thereby obtaining (4-amino-3,5-difluorophenyl)(8-bromo-2-methylimidazo[1,2-a]pyridin-3-yl)methanone (1.12 g).

### Step 6

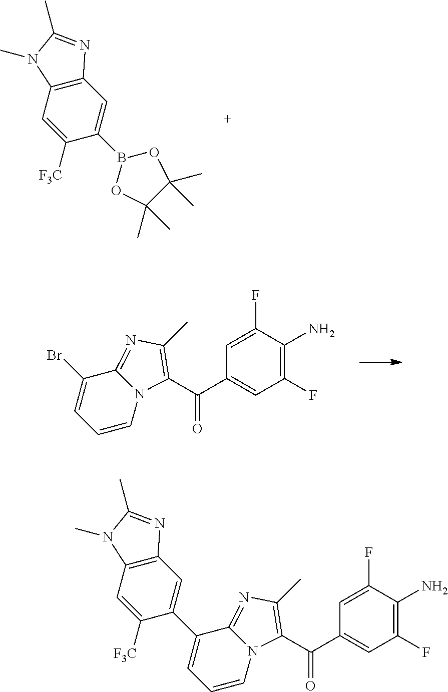

A solution of (4-amino-3,5-difluorophenyl)(8-bromo-2-methylimidazo[1,2-a]pyridin-3-yl)methanone (162 mg) obtained in step 5, 1,2-dimethyl-5-(4,4,5,5-tetramethyl-1,3,2-dioxaborolan-2-yl)-6-(trifluoromethyl)-1H-benzo[d]imidazole (116 mg), tetrakis(triphenylphosphine)palladium(0) (39.3 mg), 2.0 M sodium bicarbonate in water (340 μL) in 1,2-dimethoxyethane (1.5 mL) was stirred at 140° C. for 40 minutes using microwave irradiation. After dilution with ethyl acetate and water, the mixture was extracted with ethyl acetate. The combined organic layer was washed with water and a saturated sodium chloride solution, followed by drying over sodium sulfate. After evaporation, the obtained residue was purified by column chromatography (chloroform:ethanol), thereby obtaining (4-amino-3,5-difluorophenyl)(8-(1,2-dimethyl-6-(trifluoromethyl)-1H-benzo[d]imidazol-5-yl)-2-methlimidazo[1,2-a]pyridin-3-yl)methanone (70.0 mg).

### Step 7

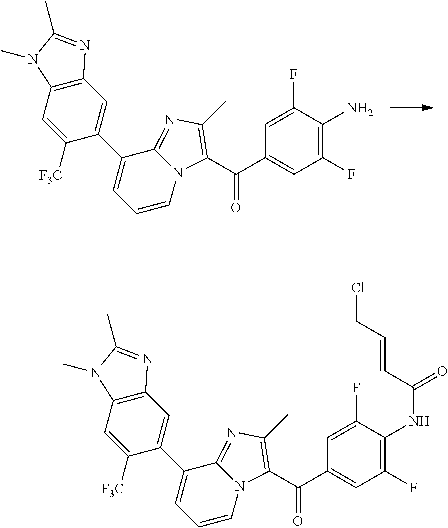 [SMILES: Cc1nc2c(-c3cc4nc(C)n(C)c4cc3C(F)(F)F)cccn2c1C(=O)c1cc(F)c(N)c(F)c1 ; Cc1nc2c(-c3cc4nc(C)n(C)c4cc3C(F)(F)F)cccn2c1C(=O)c1cc(F)c(NC(=O)/C=C/CCl)c(F)c1]

(E)-4-chlorobut-2-enoic acid (33.8 mg) and 1-propanephosphonic acid anhydride cyclic trimer (a 1.7M THF solution, 263 μL) were added to a solution of (4-amino-3,5-difluorophenyl)(8-(1,2-dimethyl-6-(trifluoromethyl)-1H-benzo[d]imidazol-5-yl)-2-methylimidazo[1,2-a]pyridin-3-yl)methanone (70.0 mg) obtained in step 6 in DMF (0.8 mL), followed by stirring at room temperature for 20 minutes. Triethylamine (117 μL) was added to the reaction mixture. A reaction was performed at room temperature for 2 hours. After water was added to the reaction mixture, extraction was performed with ethyl acetate, followed by washing the organic layer with a saturated sodium chloride solution. The washed organic layer was dried over sodium sulfate. After evaporation, the obtained residue was purified by column chromatography (chloroform:ethanol), thereby obtaining (E)-4-chloro-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide (59.7 mg).

### Step 8

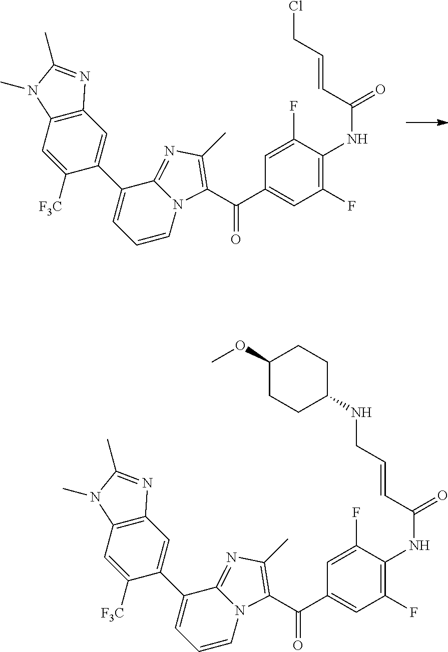 [SMILES: Cc1nc2c(-c3cc4nc(C)n(C)c4cc3C(F)(F)F)cccn2c1C(=O)c1cc(F)c(NC(=O)/C=C/CCl)c(F)c1 ; CO[C@H]1CC[C@H](NC/C=C/C(=O)Nc2c(F)cc(C(=O)c3c(C)nc4c(-c5cc6nc(C)n(C)c6cc5C(F)(F)F)cccn34)cc2F)CC1]

(1r,4r)-4-methoxycyclohexan-1-amine hydrochloride (17.6 mg), tripotassium phosphate (45.1 mg), and potassium iodide (26.5 mg) were added to a solution of (E)-4-chloro-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide (32.0 mg) obtained in step 7 in DMF (500 μL). A reaction was performed at room temperature for 2 hours. After water was added to the reaction mixture, extraction was performed with ethyl acetate. The combined organic layer was washed with a saturated sodium chloride solution. After the washed layer was dried over sodium sulfate, the solvent was evaporated under reduced pressure. The residue was purified by preparative reversed-phase HPLC (water:acetonitrile (0.1% formic acid)). Saturated sodium bicarbonate water was added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (11.0 mg).

### Example 52

### (E)-4-(tert-butylamino)-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide

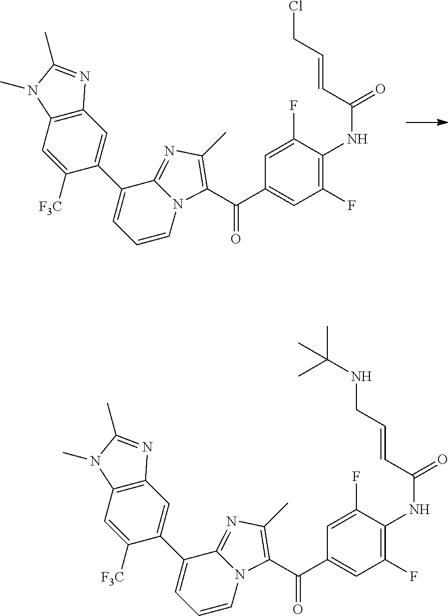 [SMILES: Cc1nc2c(-c3cc4nc(C)n(C)c4cc3C(F)(F)F)cccn2c1C(=O)c1cc(F)c(NC(=O)/C=C/CCl)c(F)c1 ; Cc1nc2c(-c3cc4nc(C)n(C)c4cc3C(F)(F)F)cccn2c1C(=O)c1cc(F)c(NC(=O)/C=C/CNC(C)(C)C)c(F)c1]

tert-Butylamine (18 μL), tripotassium phosphate (29.2 mg), and potassium iodide P322C3 (22.8 mg) were added to a solution of (E)4-chloro-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide (27.6 mg) obtained in Example 51 (step 7) in DMF (500 μL). A reaction was performed at room temperature for 2 hours. After water was added to the reaction mixture, extraction was performed with ethyl acetate. The combined organic layer was washed with a saturated sodium chloride solution. After the washed layer was dried over sodium sulfate, the solvent was evaporated under reduced pressure. The residue was purified by preparative reversed-phase HPLC (water:acetonitrile (0.1% formic acid)). Saturated sodium bicarbonate water was added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (8.93 mg).

### Example 53

### (E)-4-(tert-butylamino)-N-(2,6-difluoro-4-(8-(4-methoxy-1,2-dimethyl-6-(trifluoromethyl)-1H-benzo[d]imidazol-5-yl)indolizine-3-carbonyl)phenyl)but-2-enamide

### Step 1

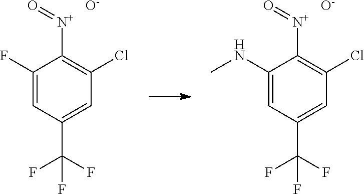

Methylamine (2M THF solution, 31.0 mL, 61.6 mmol) was added to a solution of 1-chloro-3-fluoro-2-nitro-5-(trifluoromethyl)benzene (5.00 g. 20.5 mmol) in THF (6.25 mL). After stirring at room temperature for 1 hour, the reaction mixture was concentrated under reduced pressure, thereby obtaining crude 3-chloro-N-methyl-2-nitro-5-(trifluoromethyl)aniline (5.23 g. 61.6 mmol).

### Step 2

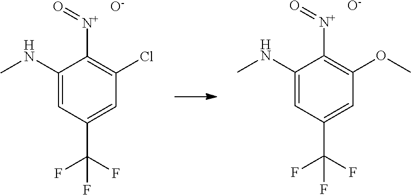

Sodium Methoxide (17.4 g. 323 mmol) was added to a solution of 3-chloro-N-methyl-2-nitro-5-(trifluoromethyl)aniline (8.22 g, 32.3 mmol) in MeOH (108 mL). After stirring at 80° C. for 24 hours, the reaction mixture was diluted with EtOAc, washed with saturated aqueous NH₄Cl and brine then dried over Na₂SO₄ and evaporated. The residue was purified by column chromatography on silica gel (EtOAc-hexane), thereby obtaining 3-methoxy-N-methyl-2-nitro-5-(trifluoromethyl)aniline (7.08 g, 284 mmol).

### Step 3

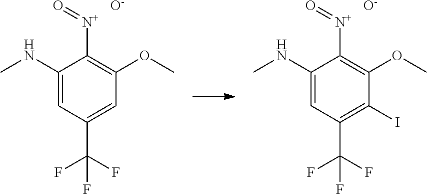

N-Iodosuccinimide (7.00 g, 31.1 mmol) was added to a solution of 3-methoxy-N-methyl-2-nitro-5-(trifluoromethyl)aniline (7.08 g, 28.3 mmol) in AcOH (70.0 mL). After stirring at 40° C. for 14 hours, the mixture was cooled to room temperature and H₂O was added to reaction mixture. The resulting solid was collected by filtration, and vacuum-dried at 60° C. for 4 hours, thereby obtaining crude 4-iodo-3-methoxy-N-methyl-2-nitro-5-(trifluoromethyl)aniline (10.3 g. 25.7 mmol).

### Step 4

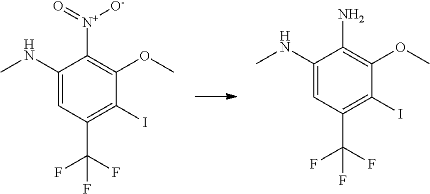

NH4Cl (3.67 g, 68.5 mmol) and iron (7.65 g, 137 mmol) were added to a solution of 4-iodo-3-methoxy-N-methyl-2-nitro-5-(trifluoromethyl)aniline (10.3 g. 27.4 mmol) in MeOH (39.0 mL), THF (39.0 mL) and H₂O (19.5 mL). After stirring at 60° C. for 2 hours, the mixture was cooled to ambient temperature and EtOAc was added to the reaction mixture then filtered through a Celite pad. The filtrate was washed with H₂O and brine then dried over Na₂SO₄ and evaporated. The residue was purified by column chromatography on silica gel (EtOAc-hexane), thereby obtaining 4-iodo-3-methoxy-N1-methyl-5-(trifluoromethyl)benzene-1,2-diamine (7.77 g, 22.7 mmol).

### Step 5

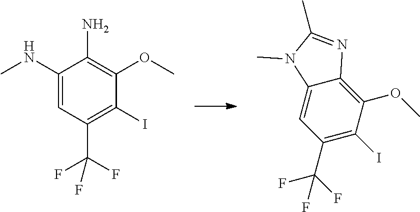

conc. HCl (187 μL, 2.25 mmol) and AcOH (1.29 mL, 22.5 mmol) were added to a solution of 4-iodo-3-methoxy-N1-methyl-5-(trifluoromethyl)benzene-1,2-diamine (7.77 g. 22.5 mmol) in trimethyl orthoacetate (40.0 mL, 314 mmol). After stirring at ambient temperature for 62 hours, the reaction was quenched with 5M aqueous NaOH (5.45 mL, 27.3 mmol). The reaction mixture was diluted with EtOAc, washed with H₂O and brine then dried over Na₂SO₄ and evaporated. The residue was dissolved in EtOAc (8.00 mL) at 50° C. and heptane (32.0 mL) was added then cooled to ambient temperature slowly to give the precipitation. The precipitate was collected by filtration, washing with further heptane, and vacuum-dried at 60° C. for 2 hours, thereby obtaining 5-iodo-4-methoxy-1,2-dimethyl-6-(trifluoromethyl)-1H-benzo[d]imidazole (5.50 g. 14.9 mmol). The filtrate was evaporated and purified by column chromatography on silica gel (EtOAc-hexane), thereby obtaining 5-iodo-4-methoxy-1,2-dimethyl-6-(trifluoromethyl)-1H-benzo[d]imidazole (1.56 g, 4.28 mmol).

### Step 6

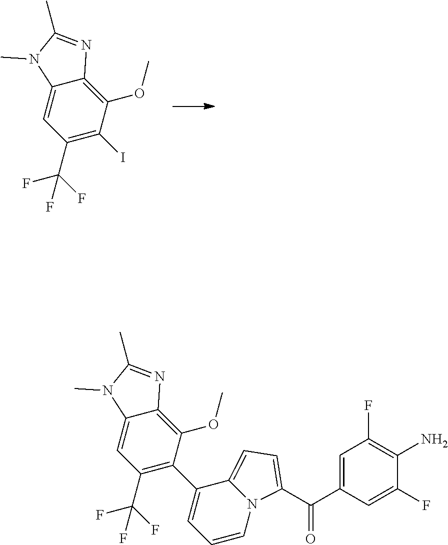

To a mixture of 5-iodo-4-methoxy-1,2-dimethyl-6-(trifluoromethyl)-1H-benzo[d]imidazole (414 mg, 1.68 mmol). (4-amino-3,5-difluorophenyl)(8-(5,5-dimethyl-1,3,2-dioxaborinan-2-yl)indolizin-3-yl)methanone (645 mg, 1.68 mmol) and Pd(PPh₃)4 (129 mg, 0.118 mmol) in 1,2-dimethoxyethane (6.2 mL) was added 2M aqueous sodium carbonate solution (2.24 mL, 4.47 mmol). After reacting by microwave apparatus at 120° C. for 2 hours, the mixture was diluted with EtOAc and water. The organic layer was washed successively with H₂O then brine, dried over sodium sulfate, and evaporated. The residue was purified by column chromatography on silica gel (acetone-hexane), thereby obtaining (4-amino-3,5-difluorophenyl)(8-(4-methoxy-1,2-dimethyl-6-(trifluoromethyl)-1H-benzo[d]imidazol-5-yl)indolizin-3-yl)methanone (543 mg, 1.06 mmol).

### Step 7

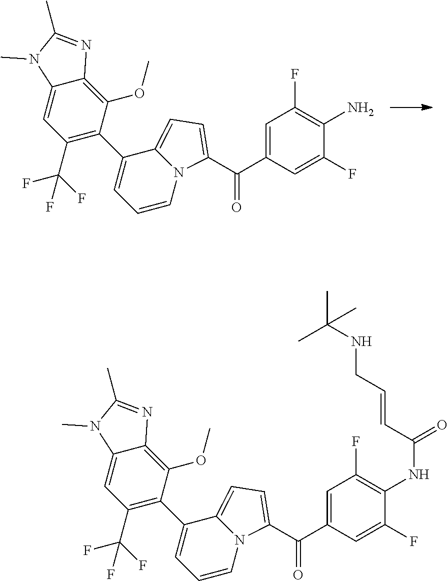 [SMILES: COc1c(-c2cccn3c(C(=O)c4cc(F)c(N)c(F)c4)ccc23)c(C(F)(F)F)cc2c1nc(C)n2C ; COc1c(-c2cccn3c(C(=O)c4cc(F)c(NC(=O)/C=C/CNC(C)(C)C)c(F)c4)ccc23)c(C(F)(F)F)cc2c1nc(C)n2C]

A solution of (4-amino-3,5-difluorophenyl)(8-(4-methoxy-1,2-dimethyl-6-(trifluoromethyl)-1H-benzo[d]imidazol-5-yl)indolizin-3-yl)methanone (90.0 mg, 0.182 mmol). 4-chlorocrotonic acid (42.2 mg, 0.364 mmol) and propylphosphonic anhydride solution (50 wt % in THF, 146 μL, 0.546 mmol) in DMF (1.00 mL) was stirred at ambient temperature for 20 minutes. Then triethylamine (146 μL, 1.05 mmol) was added thereto, and the mixture was stirred at 35° C. for 30 minutes. To the resulting mixture was added saturated aqueous NaHCO₃, and extracted with EtOAc. and washed successively with H₂O then brine, dried over sodium sulfate, and evaporated. The residue was used for next reaction without further purification.

A solution of (E)-4-chloro-N-(2,6-difluoro-4-(8-(4-methoxy-1,2-dimethyl-6-(trifluoromethyl)-1H-benzo[d]imidazol-5-yl)indolizine-3-carbonyl)phenyl)but-2-enamide (36.0 mg, 0.0587 mmol), tert-butylamine (12.2 μL, 0.124 mmol), and KI (29.1 mg, 0.182 mmol) in DMF (1.00 mL) was stirred at 35° C. for 1 hour. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (7.45 mg, 0.0112 mmol).

### Example 54

### (E)-N-(4-(8-(1,2-dimethyl-6-(trifluoromethyl)-1H-benzo[d]imidazol-5-yl)indolizine-3-carbonyl)-2,6-difluorophenyl)-4-(((1r,3r)-3-methoxycyclobutyl)amino)but-2-enamide

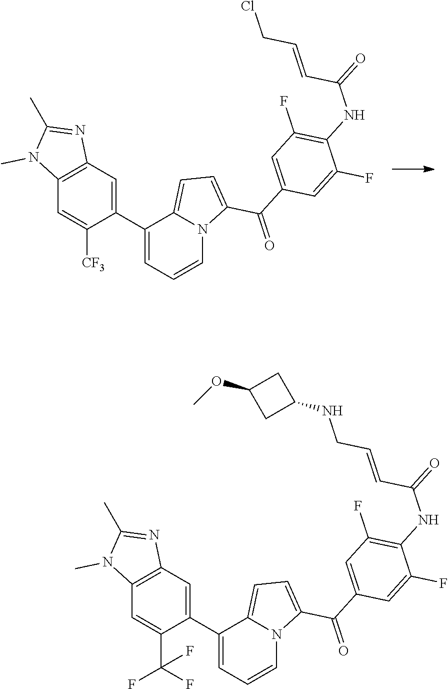 [SMILES: Cc1nc2cc(-c3cccn4c(C(=O)c5cc(F)c(NC(=O)/C=C/CCl)c(F)c5)ccc34)c(C(F)(F)F)cc2n1C ; CO[C@H]1C[C@H](NC/C=C/C(=O)Nc2c(F)cc(C(=O)c3ccc4c(-c5cc6nc(C)n(C)c6cc5C(F)(F)F)cccn34)cc2F)C1]

The procedure of Example 2 (Step 3) was performed except that trans 3-methoxycyclobutanamine hydrochloride (17.6 mg) was used instead of trans-4-methoxycyclohexanamine hydrochloride in Example 2 (Step 3), thereby obtaining the title compound (30.2 mg).

### Example 55

### (E)-N-(4-(8-(1,2-dimethyl-6-(trifluoromethyl)-1H-benzo[d]imidazol-5-indolizine-3-carbonyl)-2,6-difluorophenyl)-4-(((1r,4r)-4-hydroxycyclohexyl)amino)but-2-enamide

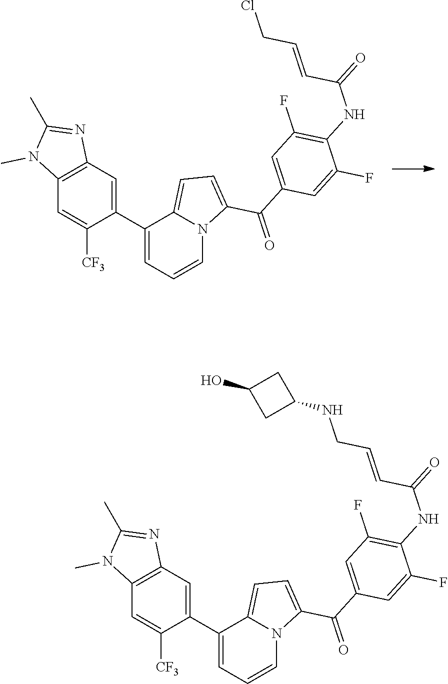 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CN[C@H]6C[C@H](O)C6)c(F)c5)ccc34)c(C(F)(F)F)cc2n1C]

The procedure of Example 2 (Step 3) was performed except that trans 4-aminocyclohexanol hydrochloride (19.4 mg) was used instead of trans-4-methoxycyclohexanamine hydrochloride in Example 2 (Step 3), thereby obtaining the title compound (25.2 mg).

### Example 56-1 and 56-2

### (E)-4-(tert-butylamino)-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide

### Step 1

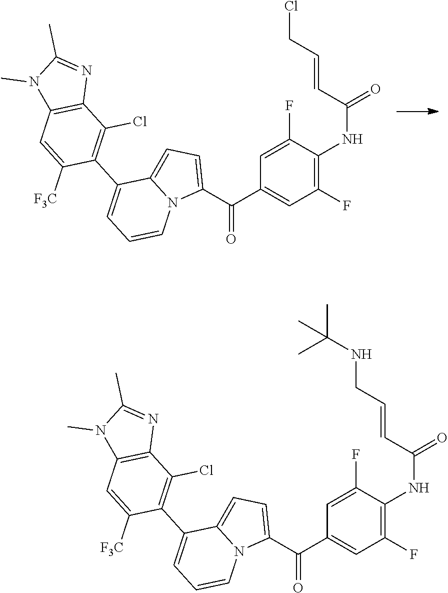 [SMILES: Cc1nc2c(Cl)c(-c3cccn4c(C(=O)c5cc(F)c(NC(=O)/C=C/CCl)c(F)c5)ccc34)c(C(F)(F)F)cc2n1C ; Cc1nc2c(Cl)c(-c3cccn4c(C(=O)c5cc(F)c(NC(=O)/C=C/CNC(C)(C)C)c(F)c5)ccc34)c(C(F)(F)F)cc2n1C]

The procedure of Example 52 was performed except that (E)-4-chloro-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide (105 mg) obtained in step 3 (Example 32) was used instead of (E)-4-chloro-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide used in Example 52, thereby obtaining the title compound (25.0 mg).

### Step 2

(E)-4-(tert-butylamino)-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide (25.0 mg) obtained in step 1 was dissolved in EtOH and purified by preparative HPLC (CHIRALPAK ID, hexane-ethanol (0.1% triethyl amine)) to afford (E)-4-(tert-butylamino)-N-(4-(8-(4-chloro-1,2-dimethyl-6-trifluoromethyl)-1H-benzo[d]imidazol-5-yl)indolizine-3-carbonyl)-2,6-difluorophenyl)but-2-enamide (Example 56-1, 10.5 mg, single isomer, 1^{st}-eluting isomer) and (E)-4-(tert-butylamino)-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide (Example 56-2, 10.5 mg, single isomer, 2^{nd}-eluting isomer).

### Example 57-1 and 57-2

### (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((tetrahydro-2H-pyran-4-yl)amino)but-2-enamide

### Step 1

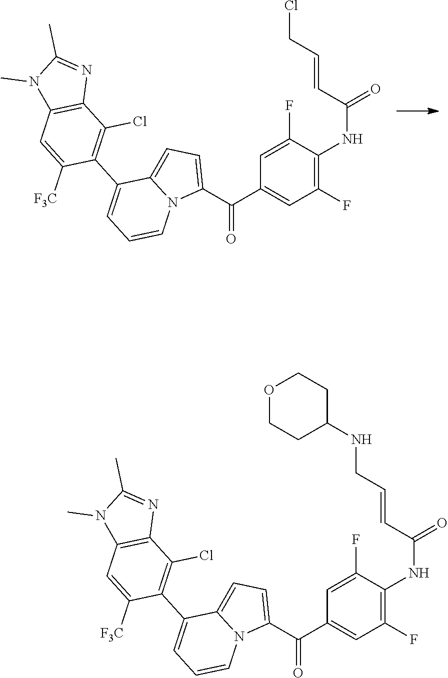 [SMILES: Cc1nc2c(Cl)c(-c3cccn4c(C(=O)c5cc(F)c(NC(=O)/C=C/CCl)c(F)c5)ccc34)c(C(F)(F)F)cc2n1C ; Cc1nc2c(Cl)c(-c3cccn4c(C(=O)c5cc(F)c(NC(=O)/C=C/CNC6CCOCC6)c(F)c5)ccc34)c(C(F)(F)F)cc2n1C]

The procedure of Example 52 was performed except that (E)-4-chloro-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide (69 mg) obtained in step 3 (Example 32) and tetrahydro-2H-pyran-4-amine were used instead of (E)-4-chloro-N-(4-(8-(1,2-dimethyl-6-(trifluoromethyl)-1H-benzo[d]imidazol-5-yl)-2-methylimidazo[1,2-a]pyridine-3-carbonyl)-2,6-difluorophenyl)but-2-enamide used in Example 52 and (1r,4r)-4-methoxycyclohexan-1-amine hydrochloride, thereby obtaining the title compound (10.0 mg).

### Step 2

(E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((tetrahydro-2H-pyran-4-yl)amino)but-2-enamide (10.0 mg) obtained in step 1 was dissolved in EtOH and purified by preparative HPLC (CHIRAL ART SB, hexane-ethanol (0.1% triethyl amine)) to afford (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((tetrahydro-2H-pyran-4-yl)amino)but-2-enamide (Example 57-1, 3.48 mg, single isomer. 1^{st}-eluting isomer) and (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((tetrahydro-2H-pyran-4-yl)amino)but-2-enamide (Example 57-2, 3.07 mg, single isomer, 2^{nd}-eluting isomer).

### Example 58

### (E)-N-(4-(8-(4-chloro-2-(hydroxymethyl)-1,6-dimethyl-1H-benzo[d]imidazol-5-yl)indolizine-3-carbonyl)-2,6-difluorophenyl-4-(((1r,4r)-4-methoxycyclohexyl)amino)but-2-enamide

The procedure of Example 58 was performed in the same manner as Example 59 to give the title compound.

### Example 59

### (4-chloro-5-(3-(3,5-difluoro-4-((E)-4-(((1r,4r)-4-methoxycyclohexyl)amino)but-2-enamido)benzoyl)indolizin-8-yl)-1,6-dimethyl-1H-benzo[d]imidazol-2-yl)methyl (E)-4-(((1r,4r)-4-methoxycyclohexyl)amino)but-2-enoate

### Step 1

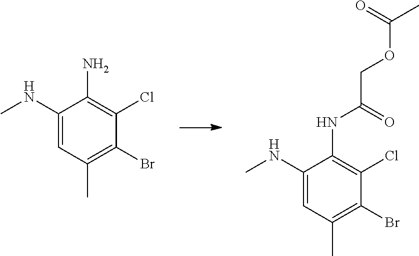 [SMILES: CNc1cc(C)c(Br)c(Cl)c1N ; CNc1cc(C)c(Br)c(Cl)c1NC(=O)COC(C)=O]

A solution of 4-bromo-3-chloro-N1,5-dimethylbenzene-1,2-diamine (100 mg), 2-acetoxyacetic acid (61.5 mg, 0.521 mmol), propylphosphonic anhydride solution (50 wt % in THF, 0.626 mL, 1.00 mmol) was stirred at RT for 15 min. Then triethylamine (0.223 mL, 1.60 mmol) was added thereto, and the mixture was stirred at RT overnight. To the resulting mixture was added EtOAc, saturated aqueous NaHCO₃, and water, and the organic layer was washed successively with H₂O, and brine, dried over sodium sulfate, and evaporated to give crude 2-((3-bromo-2-chloro-4-methyl-6-(methylamino)phenyl)amino)-2-oxoethyl acetate (141 mg).

### Step 2

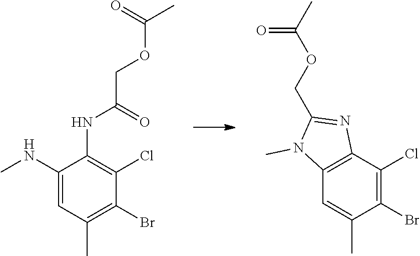 [SMILES: CNc1cc(C)c(Br)c(Cl)c1NC(=O)COC(C)=O ; CC(=O)OCc1nc2c(Cl)c(Br)c(C)cc2n1C]

The procedure of Example 47.48 (Step 2) was performed except that crude 2-((3-bromo-2-chloro-4-methyl-6-(methylamino)phenyl)amino)-2-oxoethyl acetate (141 mg) was used instead of (R)—N-(3-bromo-2-chloro-4-methyl-6-(methylamino)phenyl)-2-methoxypropanamide in Example 47, 48 (Step 2), thereby obtaining (5-bromo-4-chloro-1,6-dimethyl-1H-benzo[d]imidazol-2-yl)methyl acetate (92.5 mg).

### Step 3

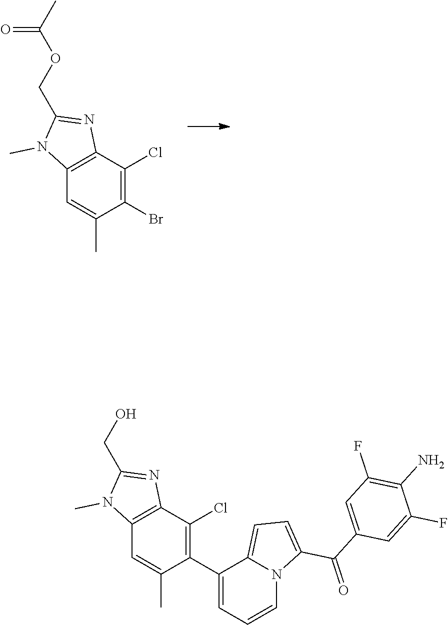 [SMILES: CC(=O)OCc1nc2c(Cl)c(Br)c(C)cc2n1C ; Cc1cc2c(nc(CO)n2C)c(Cl)c1-c1cccn2c(C(=O)c3cc(F)c(N)c(F)c3)ccc12]

To a mixture of (4-amino-3,5-difluorophenyl)(8-(5,5-dimethyl-1,3,2-dioxaborinan-2-yl)indolizin-3-yl)methanone (139 mg,), (5-bromo-4-chloro-1,6-dimethyl-1H-benzo[d]imidazol-2-yl)methyl acetate (90.0 mg) and Pd(PPh₃)₄ (31.4 mg) in 1,2-dimethoxyethane (2.0 mL) was added 2M aqueous sodium carbonate solution (0.271 mL). After reacting by microwave apparatus at 120° C. for 3 hours. 2M aqueous sodium carbonate solution (0.271 mL) was added. After reacting by microwave apparatus at 120° C. for 1 hour, the mixture was diluted with water, and extracted with EtOAc. The organic layer was washed with brine, dried over sodium sulfate and evaporated under reduced pressure. The residue was purified by column chromatography on silica gel (CHCl₃-methanol) to give (4-amino-3,5-difluorophenyl)(8-(4-chloro-2-(hydroxymethyl)-1,6-dimethyl-1H-benzo[d]imidazol-5-yl)indolizin-3-yl)methanone (102 mg).

### Step 4

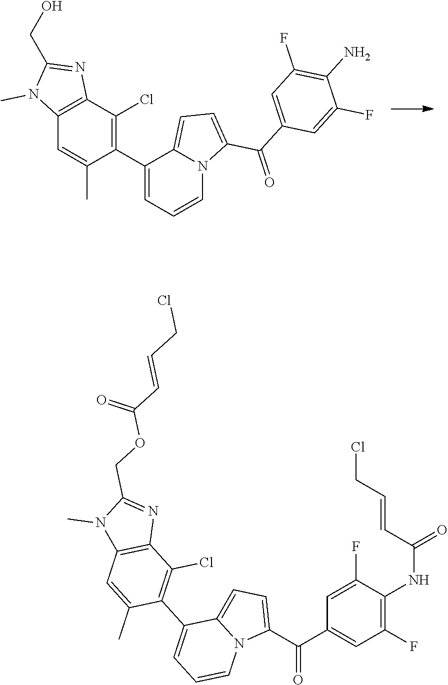 [SMILES: Cc1cc2c(nc(CO)n2C)c(Cl)c1-c1cccn2c(C(=O)c3cc(F)c(N)c(F)c3)ccc12 ; Cc1cc2c(nc(COC(=O)/C=C/CCl)n2C)c(Cl)c1-c1cccn2c(C(=O)c3cc(F)c(NC(=O)/C=C/CCl)c(F)c3)ccc12]

The procedure of Example 2 (step 2) was performed except that (4-amino-3,5-difluorophenyl)(8-(4-chloro-2-(hydroxymethyl)-1,6-dimethyl-1H-benzo[d]imidazol-5-yl)indolizin-3-yl)methanone (100 mg) obtained in step 3 was used instead of (4-amino-3,5-difluorophenyl)(8-(1,2-dimethyl-6-(trifluoromethyl)-1H-benzo[d]imidazol-5-yl)indolizin-3-yl)methanone used in Example 2 (step 2), thereby obtaining (4-chloro-5-(3-(4-((E)-4-chlorobut-2-enamido)-3,5-difluorobenzoyl)indolizin-8-yl)-1,6-dimethyl-1H-benzo[d]imidazol-2-yl)methyl (E)-4-chlorobut-2-enoate (6.60 mg).

### Step 5

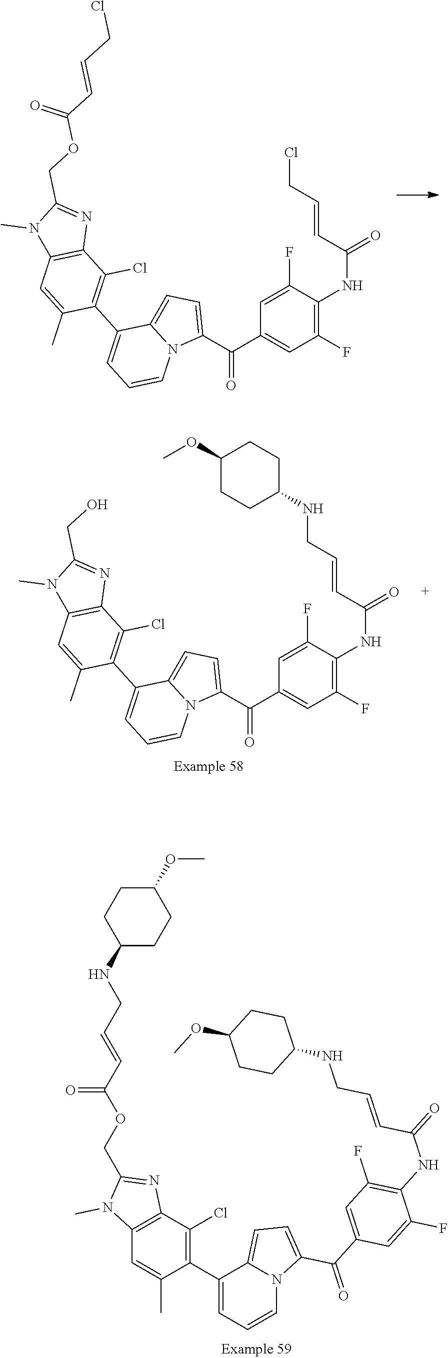 [SMILES: Cc1cc2c(nc(COC(=O)/C=C/CCl)n2C)c(Cl)c1-c1cccn2c(C(=O)c3cc(F)c(NC(=O)/C=C/CCl)c(F)c3)ccc12 ; CO[C@H]1CC[C@H](NC/C=C/C(=O)Nc2c(F)cc(C(=O)c3ccc4c(-c5c(C)cc6c(nc(CO)n6C)c5Cl)cccn34)cc2F)CC1 ; CO[C@H]1CC[C@H](NC/C=C/C(=O)Nc2c(F)cc(C(=O)c3ccc4c(-c5c(C)cc6c(nc(COC(=O)/C=C/CN[C@H]7CC[C@H](OC)CC7)n6C)c5Cl)cccn34)cc2F)CC1]

A solution of (4-chloro-5-(3-(4-((E)-4-chlorobut-2-enamido)-3,5-difluorobenzoyl)indolizin-8-yl)-1,6-dimethyl-1H-benzo[d]imidazol-2-yl)methyl (E)-4-chlorobut-2-enoate (6.60 mg), trans-4-methoxycyclohexanamine hydrochloride (3.98 mg. 0.0241 mmol), KI (4.79 mg, 0.0289 mmol) and K₃PO₄ (10.2 mg, 0.0481 mmol) in DMF (1.0 mL) was stirred at 35° C. for 7 hours and at RT overnight. 7M NH₃ in MeOH (0.2 ml) was added and the mixture was stirred at RT for 40 min. 7M NH₃ in MeOH (0.3 ml) was added and the mixture was stirred at RT for 40 min and at 35° C. for 90 min. To the resulting mixture was added H₂O and EtOAc. and the organic layer was washed successively with H₂O then brine, dried over sodium sulfate, and evaporated. The residue was purified by preparative HPLC (water:acetonitrile (0.1% formic acid)). Saturated sodium bicarbonate water and H₂O were added to the purified fractions containing the title compound, followed by extraction with ethyl acetate. The extract was washed with a saturated sodium chloride solution and dried over sodium sulfate. The solvent was evaporated under reduced pressure, thereby obtaining the title compound (Example 58; 1.53 mg. Example 59; 2.08 mg).

### Example 60-1 and 60-2

### (E)-N-(2,6-difluoro-4-(8-(4-methoxy-1,2-dimethyl-6-(trifluoromethyl)-1H-benzo[d]imidazol-5-yl)indolizine-3-carbonyl)phenyl)-4-(((1r,4r)-4-methoxycyclohexyl)amino)but-2-enamide

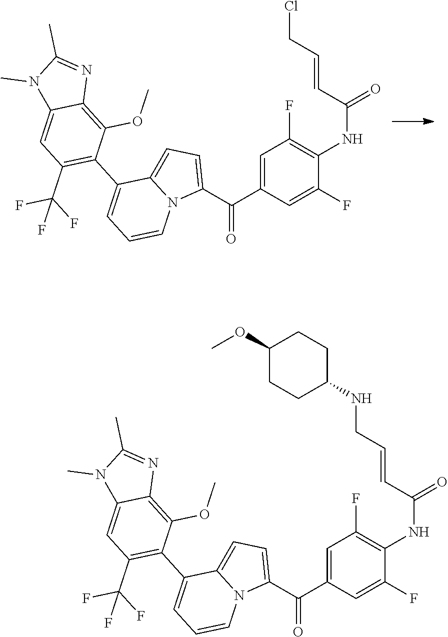 [SMILES: COc1c(-c2cccn3c(C(=O)c4cc(F)c(NC(=O)/C=C/CCl)c(F)c4)ccc23)c(C(F)(F)F)cc2c1nc(C)n2C ; COc1c(-c2cccn3c(C(=O)c4cc(F)c(NC(=O)/C=C/CN[C@H]5CC[C@H](OC)CC5)c(F)c4)ccc23)c(C(F)(F)F)cc2c1nc(C)n2C]

A solution of (E)-4-chloro-N-(2,6-difluoro-4-(8-(4-methoxy-1,2-dimethyl-6-(trifluoromethyl)-1H-benzo[d]imidazol-5-yl)indolizine-3-carbonyl)phenyl)but-2-enamide (180 mg, 0.292 mmol), trans-4-methoxycyclohexanamine hydrochloride (96.7 mg, 0.584 mmol), KI (145 mg, 0.887 mmol) and K₃PO₄ (248 mg, 1.17 mmol) in DMF (5.00 mL) was stirred at 35° C. for 3 hours. To the resulting mixture was added saturated aqueous NaHCO₃, and extracted with EtOAc, and washed successively with H₂O then brine, dried over sodium sulfate, and evaporated. The residue was purified by column chromatography on silica gel (methanol-EtOAc-hexane), thereby obtaining the racemate title compound (148 mg, 0.215 mmol). The racemate was separated by CHIRALPAK IG (hexane-ethanol(0.1% triethyl amine)) to give the title compound (53.7 mg, single isomer. 1^{st}-eluting isomer) and (50.2 mg, single isomer. 2^{nd}-eluting isomer).

### Example 61-1 and 61-2

### (E)-N-(2,6-difluoro-4-(8-(4-methoxy-1,2-dimethyl-6-(trifluoromethyl)-1H-benzo[d]imidazol-5-yl)indolizine-3-carbonyl)phenyl)-4-((tetrahydro-2H-pyran-4-yl)amino)but-2-enamide

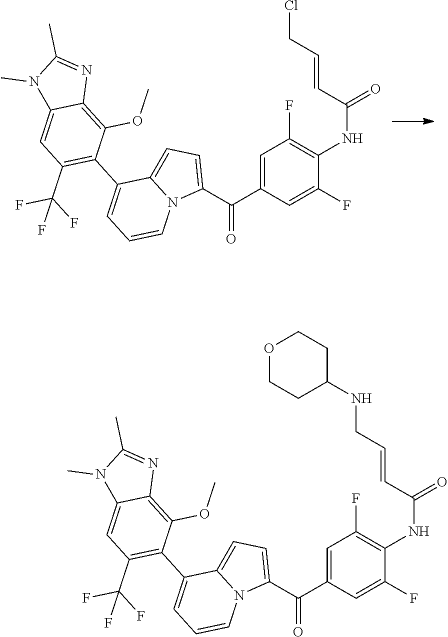 [SMILES: COc1c(-c2cccn3c(C(=O)c4cc(F)c(NC(=O)/C=C/CCl)c(F)c4)ccc23)c(C(F)(F)F)cc2c1nc(C)n2C ; COc1c(-c2cccn3c(C(=O)c4cc(F)c(NC(=O)/C=C/CNC5CCOCC5)c(F)c4)ccc23)c(C(F)(F)F)cc2c1nc(C)n2C]

A solution of (E)-4-chloro-N-(2,6-difluoro-4-(8-(4-methoxy-1,2-dimethyl-6-(trifluoromethyl)-1H-benzo[d]imidazol-5-yl)indolizine-3-carbonyl)phenyl)but-2-enamide (180 mg, 0.292 mmol). 4-aminotetrahydropyran (62.0 μL, 0.584 mmol), and KI (145 mg. 0.887 mmol) in DMF (5.00 mL) was stirred at 35° C. for 3 hours. To the resulting mixture was added saturated aqueous NaHCO₃, and extracted with EtOAc, and washed successively with H₂O then brine, dried over sodium sulfate, and evaporated. The residue was purified by column chromatography on silica gel (methanol-EtOAc-hexane), thereby obtaining the racemate title compound (137 mg, 0.202 mmol). The racemate was separated by CHIRALPAK IG (hexane-ethanol(0.1% triethyl amine)) to give the title compound (52.9 mg, single isomer. 1^{st}-eluting isomer) and (52.3 mg, single isomer, 2^{nd}-eluting isomer).

### Example 62

### (E)-N-(4-(8-(1,2-dimethyl-6-(trifluoromethyl)-1H-benzo[d]imidazol-5-yl)indolizine-3-carbonyl)-2,6-difluorophenyl)-4-((1-methylcyclopropyl)amino)but-2-enamide

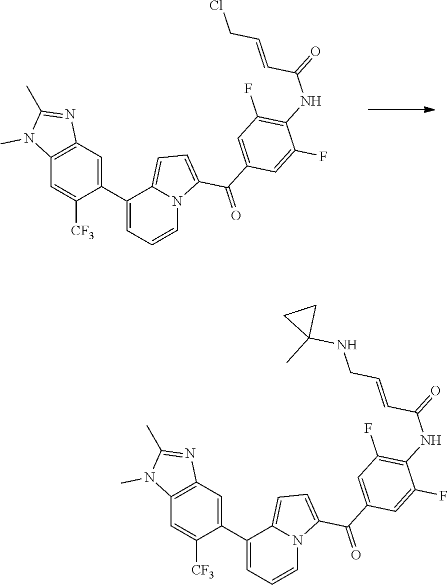 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6(C)CC6)c(F)c5)ccc34)c(C(F)(F)F)cc2n1C]

The procedure of Example 2 (Step 3) was performed except that 1-methylcyclopropanamine hydrochloride (9.16 mg) was used instead of trans-4-methoxycyclohexanamine hydrochloride in Example 2 (Step 3), thereby obtaining the title compound (2.66 mg).

### Example 63

### (E)-N-(4-(8-(1,2-dimethyl-6-(trifluoromethyl)-1H-benzo[d]imidazol-5-yl)indolizine-3-carbonyl)-2,6-difluorophenyl)-4-((2-hydroxycyclohexyl)amino)but-2-enamide

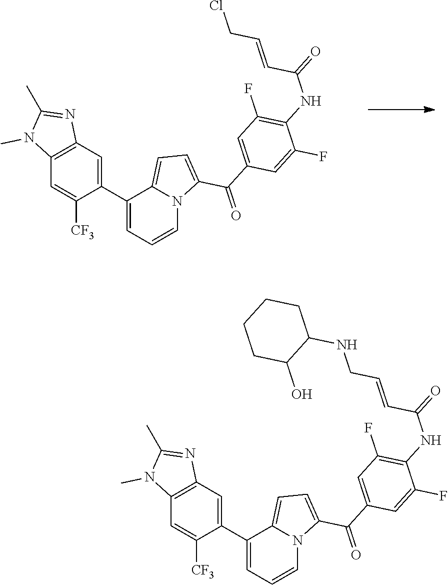 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCCCC6O)c(F)c5)ccc34)c(C(F)(F)F)cc2n1C]

The procedure of Example 2 (Step 3) was performed except that 2-aminocyclohexanol (9.81 mg) was used instead of trans-4-methoxycyclohexanamine hydrochloride in Example 2 (Step 3), thereby obtaining the title compound (4.29 mg).

### Example 64

(E)-N-(4-(8-(4-chloro-6-(difluoromethyl)-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

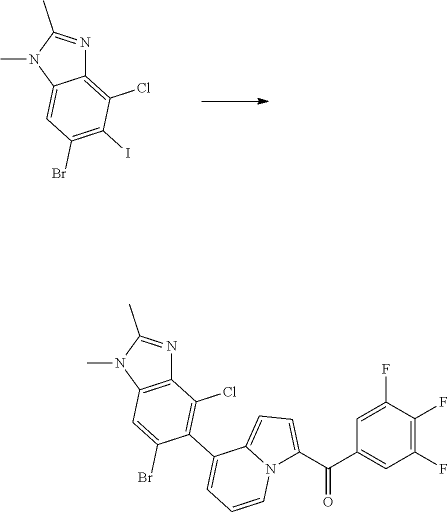 [SMILES: Cc1nc2c(Cl)c(I)c(Br)cc2n1C ; Cc1nc2c(Cl)c(-c3cccn4c(C(=O)c5cc(F)c(F)c(F)c5)ccc34)c(Br)cc2n1C]

To a mixture of 6-bromo-4-chloro-5-iodo-1,2-dimethyl-1H-benzo[d]imidazole (200 mg, 0.519 mmol), (3,4,5-trifluorophenyl)(8-(5,5-dimethyl-1,3,2-dioxaborinan-2-yl)indolizin-3-yl)methanone (281 mg, 0.726 mmol) and Pd(PPh₃)₄ (60.0 mg, 0.0519 mmol) in 1,2-dimethoxyethane (2.00 mL) was added 2M aqueous sodium carbonate solution (519 μL, 1.04 mmol). After reacting by microwave apparatus at 110° C. for 2 hours, the mixture was diluted with EtOAc and water. The organic layer was washed successively with H₂O then brine, dried over sodium sulfate, and evaporated. The residue was purified by column chromatography on silica gel (acetone-hexane), thereby obtaining (8-(6-bromo-4-chloro-1,2-dimethyl-1H-benzo[d]imidazol-5-yl)indolizin-3-yl)(3,4,5-trifluorophenyl)methanone (345 mg, 0.648 mmol).

### Step 2

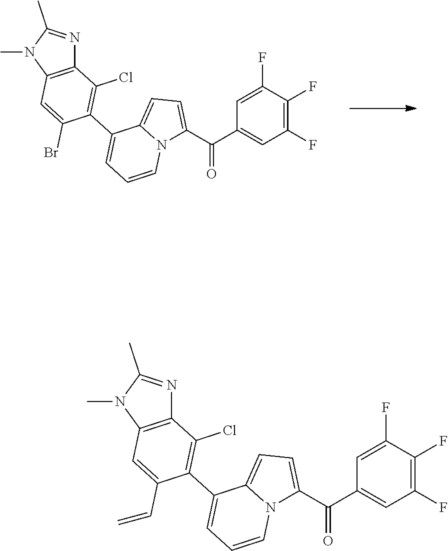 [SMILES: Cc1nc2c(Cl)c(-c3cccn4c(C(=O)c5cc(F)c(F)c(F)c5)ccc34)c(Br)cc2n1C ; C=Cc1cc2c(nc(C)n2C)c(Cl)c1-c1cccn2c(C(=O)c3cc(F)c(F)c(F)c3)ccc12]

To a mixture of (8-(6-bromo-4-chloro-1,2-dimethyl-1H-benzo[d]imidazol-5-yl)indolizin-3-yl)(3,4,5-trifluorophenyl)methanone (100 mg, 0.189 mmol), potassium vinyl trifluoroborate (75.9 mg, 0.566 mmol) and Pd(dppf)Cl₂ (15.4 mg, 0.0189 mmol) in 1,2-dimethoxyethane (700 μL) was added 2M aqueous sodium carbonate solution (330 μL, 0.661 mmol). After reacting by microwave apparatus at 100° C. for 3 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining (8-(4-chloro-1,2-dimethyl-6-vinyl-1H-benzo[d]imidazol-5-yl)indolizin-3-yl)(3,4,5-trifluorophenyl)methanone (44.2 mg, 0.0927 mmol).

### Step 3

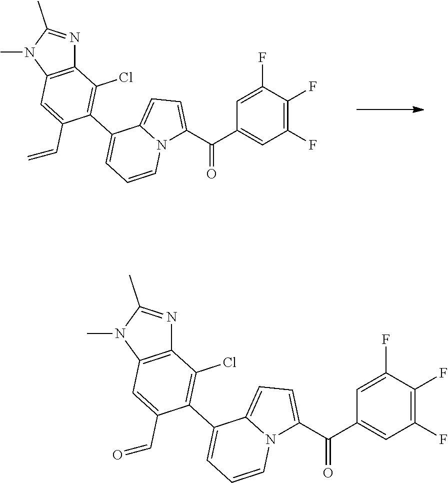 [SMILES: C=Cc1cc2c(nc(C)n2C)c(Cl)c1-c1cccn2c(C(=O)c3cc(F)c(F)c(F)c3)ccc12 ; Cc1nc2c(Cl)c(-c3cccn4c(C(=O)c5cc(F)c(F)c(F)c5)ccc34)c(C=O)cc2n1C]

A solution of (8-(4-chloro-1,2-dimethyl-6-vinyl-1H-benzo[d]imidazol-5-yl)indolizin-3-yl)(3,4,5-trifluorophenyl)methanone (87.5 mg, 0.160 mmol), osmium tetroide (9.16 mg, 0.0160 mmol) and 1,4-lutidine (37.2 μL, 0.321 mmol) in 1,4-dioxane (500 μL) was stirred at ambient temperature for 5 minutes. Then sodium periodate (137 mg, 0.642 mmol) in H₂O (100 μL) was added thereto, and the mixture was stirred at ambient temperature for 1 hour. To the resulting mixture was added saturated aqueous NaHCO₃, NaHSO₃, and extracted with EtOAc. and washed successively with H₂O then brine, dried over sodium sulfate, and evaporated. The residue was purified by column chromatography on silica gel (EtOAc-hexane), thereby obtaining 4-chloro-1,2-dimethyl-5-(3-(3,4,5-trifluorobenzoyl)indolizin-8-yl)-1H-benzo[d]imidazole-6-carbaldehyde (71.8 mg, 0.149 mmol).

### Step 4

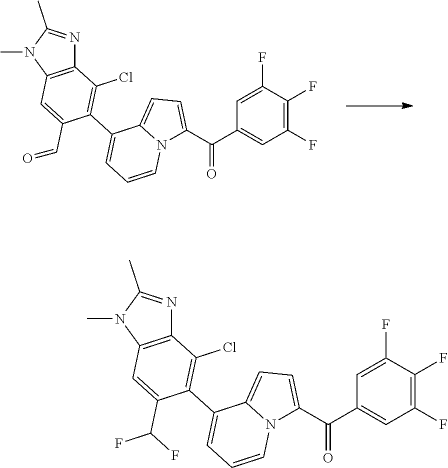 [SMILES: Cc1nc2c(Cl)c(-c3cccn4c(C(=O)c5cc(F)c(F)c(F)c5)ccc34)c(C=O)cc2n1C ; Cc1nc2c(Cl)c(-c3cccn4c(C(=O)c5cc(F)c(F)c(F)c5)ccc34)c(C(F)F)cc2n1C]

N,N-diethylaminosulfur trifluoride (197 μL, 1.49 mmol) was added to a solution of 4-chloro-1,2-dimethyl-5-(3-(3,4,5-trifluorobenzoyl)indolizin-8-yl)-1H-benzo[d]imidazole-6-carbaldehyde (81.8 mg, 0.149 mmol) in CH₂Cl₂ (300 μL), and the mixture was stirred at ambient temperature for 1 hour. To the resulting mixture was added saturated aqueous NaHCO₃, and extracted with CHCl₃, and washed successively with H₂O then brine, dried over sodium sulfate, and evaporated. The residue was purified by column chromatography on silica gel (EtOAc-hexane), thereby obtaining (8-(4-chloro-6-(difluoromethyl)-1,2-dimethyl-1H-benzo[d]imidazol-5-yl)indolizin-3-yl)(3,4,5-trifluorophenyl)methanone (39.6 mg, 0.0786 mmol).

### Step 5

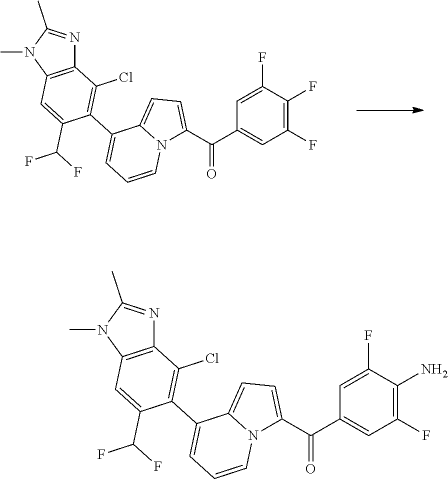 [SMILES: Cc1nc2c(Cl)c(-c3cccn4c(C(=O)c5cc(F)c(F)c(F)c5)ccc34)c(C(F)F)cc2n1C ; Cc1nc2c(Cl)c(-c3cccn4c(C(=O)c5cc(F)c(N)c(F)c5)ccc34)c(C(F)F)cc2n1C]

To a mixture of (8-(4-chloro-6-(difluoromethyl)-1,2-dimethyl-1H-benzo[d]imidazol-5-yl)indolizin-3-yl)(3,4,5-trifluorophenyl)methanone (40.0 mg, 0.0790 mmol) in 1,4-dioxane (400 μL) was added ammonium hydroxide (1.60 mL, 24.0 mmol). After reacting by microwave apparatus at 130° C. for 14 hours, the mixture was diluted with EtOAc and water. The organic layer was washed successively with H₂O then brine, dried over sodium sulfate, and evaporated. The residue was purified by column chromatography on silica gel (EtOAc-hexane), thereby obtaining (4-amino-3,5-difluorophenyl)(8-(4-chloro-6-(difluoromethyl)-1,2-dimethyl-1H-benzo[d]imidazol-5-yl)indolizin-3-yl)methanone (12.1 mg, 0.0242 mmol).

### Step 6

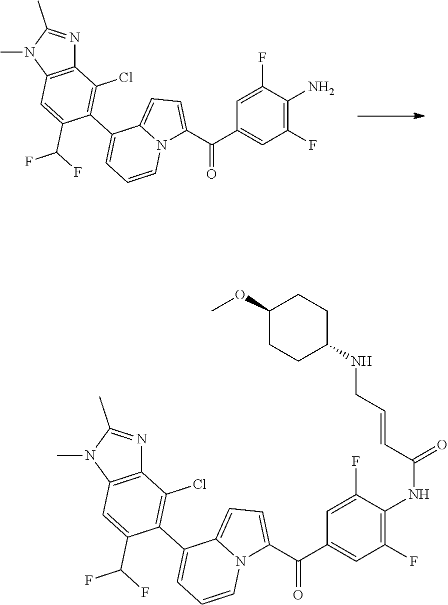 [SMILES: Cc1nc2c(Cl)c(-c3cccn4c(C(=O)c5cc(F)c(N)c(F)c5)ccc34)c(C(F)F)cc2n1C ; CO[C@H]1CC[C@H](NC/C=C/C(=O)Nc2c(F)cc(C(=O)c3ccc4c(-c5c(C(F)F)cc6c(nc(C)n6C)c5Cl)cccn34)cc2F)CC1]

A solution of (4-amino-3,5-difluorophenyl)(8-(4-chloro-6-(difluoromethyl)-1,2-dimethyl-1H-benzo[d]imidazol-5-yl)indolizin-3-yl)methanone (12.1 mg, 0.0242 mmol). 4-chlorocrotonic acid (5.82 mg, 0.0483 mmol) and propylphosphonic anhydride solution (50 wt % in THF, 45.3 μL, 0.0725 mmol) in DMF (1.00 mL) was stirred at ambient temperature for 20 minutes. Then triethylamine (20.1 μL, 0.145 mmol) was added thereto, and the mixture was stirred at 35° C. for 30 minutes. To the resulting mixture was added saturated aqueous NaHCO₃, and extracted with EtOAc, and washed successively with H₂O then brine, dried over sodium sulfate, and evaporated. The residue was used for next reaction without further purification.

A solution of (E)-4-chloro-N-(4-(8-(4-chloro-6-(difluoromethyl)-1,2-dimethyl-1H-benzo[d]imidazol-5-yl)indolizine-3-carbonyl)-2,6-difluorophenyl)but-2-enamide (14.6 mg, 0.0242 mmol), trans-4-methoxycyclohexanamine hydrochloride (8.02 mg, 0.0484 mmol). KI (12.1 mg, 0.0726 mmol) and K₃PO₄ (20.5 mg, 0.968 mmol) in DMF (1.00 mL) was stirred at 35° C. for 1 hour. To the resulting mixture was added saturated aqueous NaHCO₃, and extracted with EtOAc, and washed successively with H₂O then brine, dried over sodium sulfate, and evaporated. The residue was purified by preparative HPLC (water:acetonitrile (0.1% formic acid)) to give the title compound (3.23 mg, 0.00464 mmol).

### Example 65

(E)-N-(4-(8-(1,2-dimethyl-6-(trifluoromethyl)-1H-benzo[d]imidazol-5-yl)indolizine-3-carbonyl)-2,6-difluorophenyl)-4-((2-(hydroxy methyl)cyclohexyl)amino)but-2-enamide

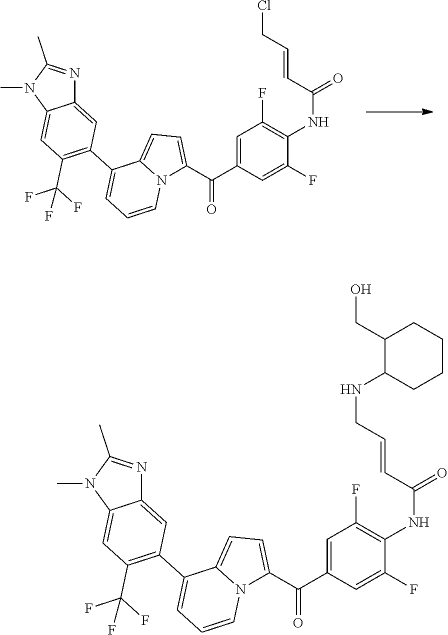 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCCCC6CO)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (12.0 mg, 0.0202 mmol), (2-aminocyclohexyl)methanol (5.30 μL, 0.0402 mmol), and KI (10.2 mg, 0.0603 mmol) in DMSO (1.00 mL) was stirred at 35° C. for 1 hour. The resulting mixture was purified by preparative HPLC (water:acetonitrile (0.1% formic acid)) to give the title compound (3.76 mg, 0.00551 mmol).

### Example 66

### (E)-N-(4-(8-(1,2-dimethyl-6-(trifluoromethyl)-1H-benzo[d]imidazol-5-yl)indolizine-3-carbonyl)-2,6-difluorophenyl)-4-(2,2-dimethyltetrahydro-2H-pyran-4-yl)amino)but-2-enamide

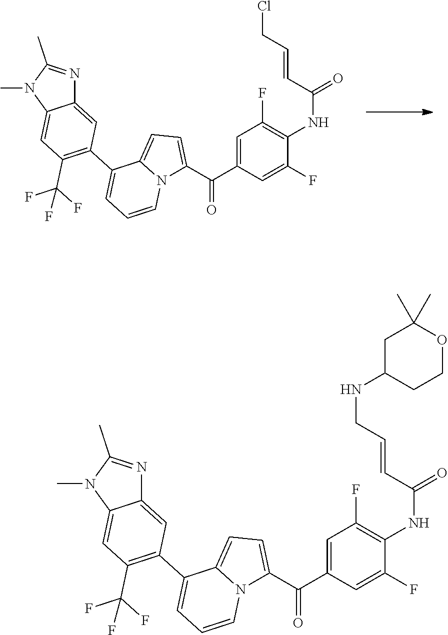 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCOC(C)(C)C6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (12.0 mg, 0.0202 mmol), 2,2-dimethyloxan-4-amine (5.0 μL, 0.0402 mmol), and KI (10.2 mg, 0.0603 mmol) in DMSO (1.00 mL) was stirred at 35° C. for 1 hour. The resulting mixture was purified by preparative HPLC (water:acetonitrile (0.1% formic acid)) to give the title compound (4.66 mg, 0.00691 mmol).

### Example 67

### (E)-N-(4-(8-(4-chloro-2-(hydroxymethyl)-1-methyl-6-(trifluoromethyl)-1H-benzo[d]imidazol-5-yl)indolizine-3-carbonyl)-2,6-difluorophenyl)-4-(((1r,4r)-4-methoxycyclohexyl)amino)but-2-enamide

The procedure of Example 67 was performed in the same manner as Example 58.

### Example 68

(E)-N-(4-(8-(4-chloro-2-(fluoromethyl)-1-methyl-6-(trifluoromethyl)-1H-benzo[d]imidazol-5-yl)indolizine-3-carbonyl)-2,6-difluorophenyl)-4-((1r,4r)-4-methoxycyclohexyl)amino)but-2-enamide

### Step 1

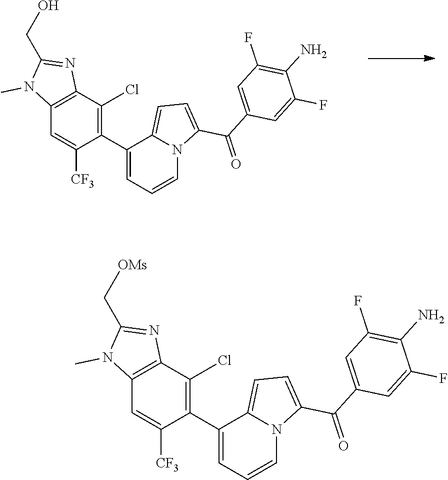 [SMILES: Cn1c(CO)nc2c(Cl)c(-c3cccn4c(C(=O)c5cc(F)c(N)c(F)c5)ccc34)c(C(F)(F)F)cc21 ; Cn1c(COS(C)(=O)=O)nc2c(Cl)c(-c3cccn4c(C(=O)c5cc(F)c(N)c(F)c5)ccc34)c(C(F)(F)F)cc21]

To a mixture of (4-amino-3,5-difluorophenyl)(8-(4-chloro-2-(hydroxymethyl)-1-methyl-6-(trifluoromethyl)-1H-benzo[d]imidazol-5-yl)indolizin-3-yl)methanone (70 mg, 0.131 mmol) and triethylamine (0.0272 mL, 0.196 mmol) in CH₂Cl₂ (2 mL) was added methanesulfonyl chloride (13.1 mL, 0.169 mmol) at 0° C. and the mixture was stirred at 0° C. for 15 min. To the resulting mixture was added EtOAc, saturated aqueous NaHCO₃, and water, and the organic layer was washed successively with saturated aqueous NaHCO₃ and brine, dried over sodium sulfate, and evaporated to give crude (5-(3-(4-amino-3,5-difluorobenzoyl)indolizin-8-yl)-4-chloro-1-methyl-6-(trifluoromethyl)-1H-benzo[d]imidazol-2-yl)methyl methanesulfonate (78.5 mg).

### Step 2

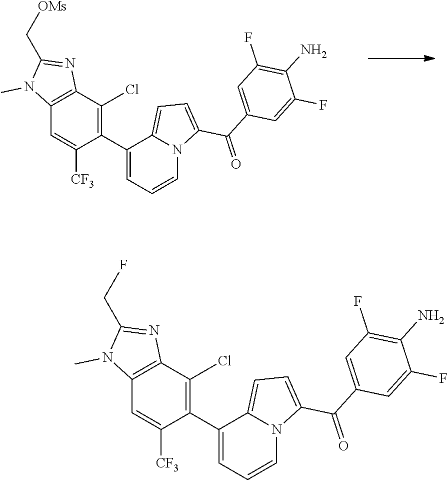 [SMILES: Cn1c(COS(C)(=O)=O)nc2c(Cl)c(-c3cccn4c(C(=O)c5cc(F)c(N)c(F)c5)ccc34)c(C(F)(F)F)cc21 ; Cn1c(CF)nc2c(Cl)c(-c3cccn4c(C(=O)c5cc(F)c(N)c(F)c5)ccc34)c(C(F)(F)F)cc21]

To a mixture of crude (5-(3-(4-amino-3,5-difluorobenzoyl)indolizin-8-yl)-4-chloro-1-methyl-6-(trifluoromethyl)-1H-benzo[d]imidazol-2-yl)methyl methanesulfonate (78.5 mg) in THF (2 mL) was added TBAF (0.128 mL, 0.128 mmol) and the mixture was stirred at RT for 60 min. To the resulting mixture was added EtOAc and water, and the organic layer was washed successively with H₂O and brine, dried over sodium sulfate, and evaporated. The residue was purified by column chromatography on silica gel (hexane-acetone) to give (4-amino-3,5-difluorophenyl)(8-(4-chloro-2-(fluoromethyl)-1-methyl-6-(trifluoromethyl)-1H-benzo[d]imidazol-5-yl)indolizin-3-yl)methanone (15.2 mg). Following steps were performed in the same manner as Example 12 to give the title compound.

### Example 69

### (E)-N-(4-(8-(1,2-dimethyl-6-(trifluoromethyl)-1H-benzo[d]imidazol-5-yl)indolizine-3-carbonyl)-2,6-difluorophenyl)-4-((1-(hydroxymethyl)cyclohexyl)amino)but-2-enamide

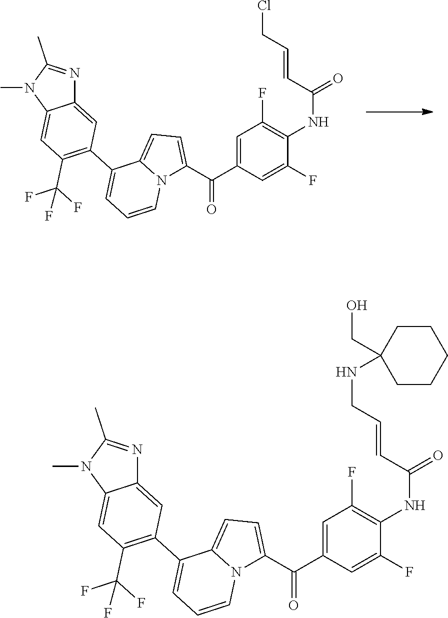 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6(CO)CCCCC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol), (1-aminocyclohexyl)methanol (6.60 μL, 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.1% formic acid)) to give the title compound (11.5 mg, 0.0172 mmol).

### Example 70

### (E)-N-(4-(8-(1,2-dimethyl-6-(trifluoromethyl)-1H-benzo[d]imidazol-5-yl)indolizine-3-carbonyl)-2,6-difluorophenyl)-4-((4-(hydroxymethyl)tetrahydro-2H-pyran-4-yl)amino)but-2-enamide

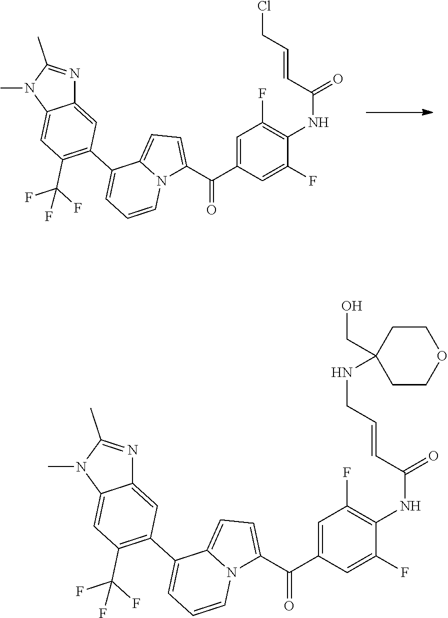 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6(CO)CCOCC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol). (4-Amino-4-tetrahydropyranyl)methanol (7.00 μL, 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.10% formic acid)) to give the title compound (9.80 mg, 0.0144 mmol).

### Example 71-1 and 71-2

### (E)-N-(4-(8-(4-chloro-6-isopropyl-1,2-dimethyl-1H-benzo[d]imidazol-5-yl)indolizine-3-carbonyl)-2,6-difluorophenyl)-4-(((1r,4r)-4-methoxycyclohexyl)amino)but-2-enamide

### Step 1

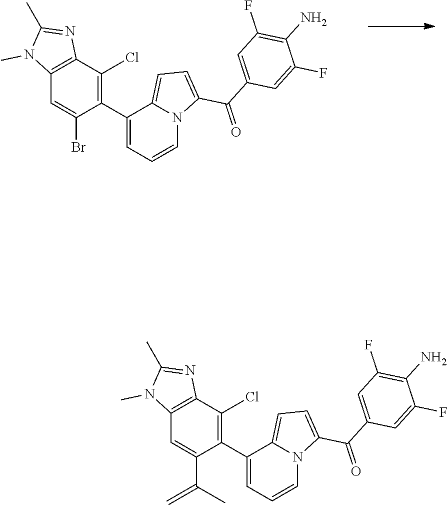 [SMILES: Cc1nc2c(Cl)c(-c3cccn4c(C(=O)c5cc(F)c(N)c(F)c5)ccc34)c(Br)cc2n1C ; C=C(C)c1cc2c(nc(C)n2C)c(Cl)c1-c1cccn2c(C(=O)c3cc(F)c(N)c(F)c3)ccc12]

To a mixture of (4-amino-3,5-difluorophenyl)(8-(6-bromo-4-chloro-1,2-dimethyl-1H-benzo[d]imidazol-5-yl)indolizin-3-yl)methanone (80.0 mg, 0.151 mmol). 2-isopropenyl-4,4,5,5-tetramethyl-1,3,2-dioxaborolane (50.8 mg, 0.302 mmol) and Pd(PPh₃)₄ (17.5 mg, 0.0151 mmol) in 1,2-dimethoxyethane (1.0 mL) was added 2M aqueous sodium carbonate solution (0.151 mL, 0.302 mmol). After reacting by microwave apparatus at 100° C. for 14 hours, the mixture was diluted with EtOAc and water. The organic layer was washed with brine, dried over sodium sulfate and evaporated under reduced pressure. The residue was purified by column chromatography on silica gel (EtOAc-hexane) to give (4-amino-3,5-difluorophenyl)(8-(4-chloro-1,2-dimethyl-6-(prop-1-en-2-yl)-1H-benzo[d]imidazol-5-yl)indolizin-3-yl)methanone (38.9 mg).

### Step 2

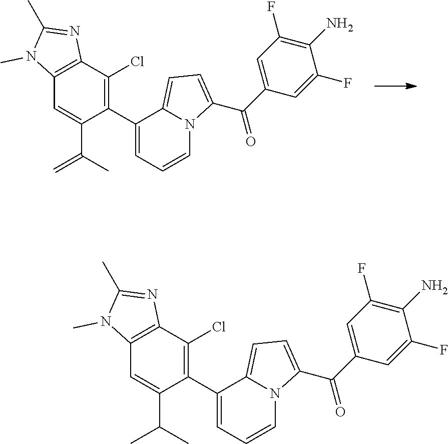 [SMILES: C=C(C)c1cc2c(nc(C)n2C)c(Cl)c1-c1cccn2c(C(=O)c3cc(F)c(N)c(F)c3)ccc12 ; Cc1nc2c(Cl)c(-c3cccn4c(C(=O)c5cc(F)c(N)c(F)c5)ccc34)c(C(C)C)cc2n1C]

A mixture of (4-amino-3,5-difluorophenyl)(8-(4-chloro-1,2-dimethyl-6-(prop-1-en-2-yl)-1H-benzo[d]imidazol-5-yl)indolizin-3-yl)methanone (38.9 mg) and Platinum on carbon (5 wt. %, 47.5 mg) in methanol (2.0 mL) was stirred at RT for 9 hours under hydrogen atmosphere. The reaction was filtered through Celite, rinsing with CHCl₃, and the filtrate was evaporated. The residue was purified by column chromatography on silica gel (EtOAc-hexane) to give (4-amino-3,5-difluorophenyl)(8-(4-chloro-6-isopropyl-1,2-dimethyl-1H-benzo[d]imidazol-5-yl)indolizin-3-yl)methanone (34.2 mg). Additional steps were performed in the same manner as Example 12 to obtain the title compound. The racemate of Example 71 was separated by CHIRALPAK IE (hexane-ethanol (0.1% triethyl amine)) to give the title compound (Example 71-1, 1.63 mg; single isomer. 1^{st}-eluting isomer and Example 71-2, 1.84 mg; single isomer. 2^{nd}-eluting isomer).

### Example 72

### (E)-N-(4-(8-(1,2-dimethyl-6-(trifluoromethyl)-1H-benzo[d]imidazol-5-yl)indolizine-3-carbonyl)-2,6-difluorophenyl)-4-((1-methyl-2-oxopiperidin-4-yl)amino)but-2-enamide

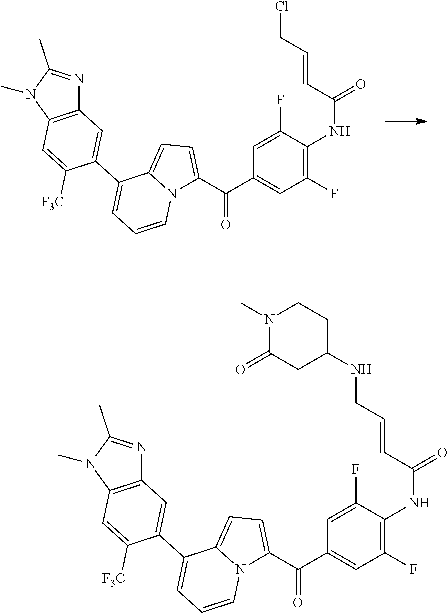 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCN(C)C(=O)C6)c(F)c5)ccc34)c(C(F)(F)F)cc2n1C]

4-amino-1-methyl-piperidin-2-one hydrochloride (14.8 mg), tripotassium phosphate (38.2 mg), and potassium iodide (22.4 mg) were added to a solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (31.0 mg) obtained in step 7 (Example 51) in DMF (500 μL). A reaction was performed at room temperature for 2 hours. After water was added to the reaction mixture, extraction was performed with ethyl acetate. The combined organic layer was washed with a saturated sodium chloride solution. After the washed layer was dried over sodium sulfate, the solvent was evaporated under reduced pressure. The residue was purified by column chromatography (chloroform:ethanol), thereby obtaining the title compound (12.0 mg).

### Example 73

### (E)-N-(4-(8-(2-(2-amino-2-oxoethyl)-4-chloro-1,6-dimethyl-1H-benzo[d]imidazol-5-yl)indolizine-3-carbonyl)-2,6-difluorophenyl)-4-(tert-butylamino)but-2-enamide

The procedure of Example 73 was performed in the same manner as Example 74.

### Example 74

### (E)-4-(tert-butylamino)-N-(4-(8-(4-chloro-2-(cyanomethyl)-1,6-dimethyl-1H-benzo[d]imidazol-5-yl)indolizine-3-carbonyl)-2,6-difluorophenyl)but-2-enamide

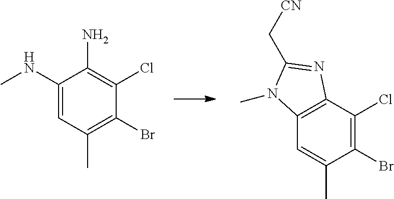 [SMILES: CNc1cc(C)c(Br)c(Cl)c1N ; Cc1cc2c(nc(CC#N)n2C)c(Cl)c1Br]

The procedure was performed in the same manner as Example 58.

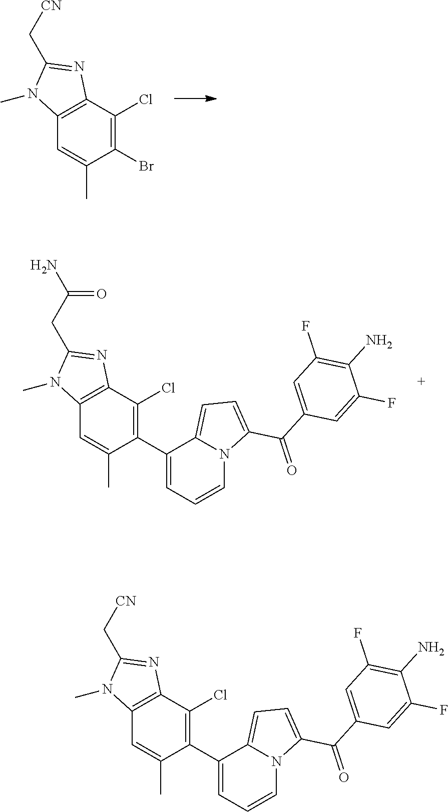 [SMILES: Cc1cc2c(nc(CC#N)n2C)c(Cl)c1Br ; Cc1cc2c(nc(CC(N)=O)n2C)c(Cl)c1-c1cccn2c(C(=O)c3cc(F)c(N)c(F)c3)ccc12 ; Cc1cc2c(nc(CC#N)n2C)c(Cl)c1-c1cccn2c(C(=O)c3cc(F)c(N)c(F)c3)ccc12]

To a mixture of (4-amino-3,5-difluorophenyl)(8-(5,5-dimethyl-1,3,2-dioxaborinan-2-yl)indolizin-3-yl)methanone (69.5 mg,), 2-(5-bromo-4-chloro-1,6-dimethyl-1H-benzo[d]imidazol-2-yl)acetonitrile (45.0 mg) and Pd(PPh3)4 (17.4 mg) in 1,2-dimethoxyethane (0.50 mL) was added 2M aqueous sodium carbonate solution (0.151 mL). After reacting by microwave apparatus at 120° C. for 3 hours, the mixture was diluted with water and EtOAc. The organic layer was washed with brine, dried over sodium sulfate and evaporated under reduced pressure. The residue was purified by column chromatography on silica gel (CHCl₃-methanol) to give 2-(5-(3-(4-amino-3,5-difluorobenzoyl)indolizin-8-yl)-4-chloro-1,6-dimethyl-1H-benzo[d]imidazol-2-yl)acetamide (22.1 mg) and 2-(5-(3-(4-amino-3,5-difluorobenzoyl)indolizin-8-yl)-4-chloro-1,6-dimethyl-1H-benzo[d]imidazol-2-yl)acetonitrile (39.2 mg). Following steps were performed in the same manner as Example 18, and Example 73 was obtained from 2-(5-(3-(4-amino-3,5-difluorobenzoyl)indolizin-8-yl)-4-chloro-1,6-dimethyl-1H-benzo[d]imidazol-2-yl)acetamide and Example 74 was obtained from 2-(5-(3-(4-amino-3,5-difluorobenzoyl)indolizin-8-yl)-4-chloro-1,6-dimethyl-1H-benzo[d]imidazol-2-yl)acetonitrile.

### Example 75

(E)-N-(4-(8-(4-chloro-1,2,6-trimethyl-1H-benzo[d]imidazol-5-yl)-1-iodoindolizine-3-carbonyl)-2,6-difluorophenyl)-4-(((1r,4r)-4-methoxycyclohexyl)amino)but-2-enamide

### Step 1

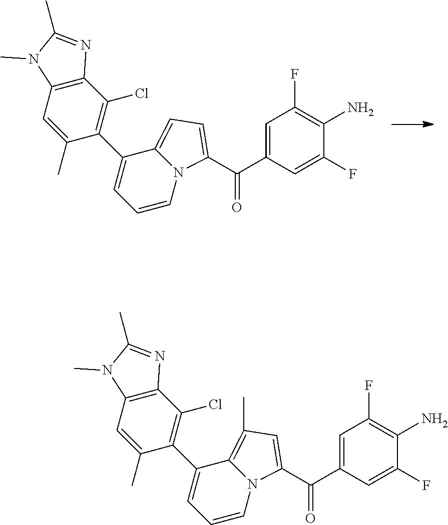 [SMILES: Cc1cc2c(nc(C)n2C)c(Cl)c1-c1cccn2c(C(=O)c3cc(F)c(N)c(F)c3)ccc12 ; Cc1cc2c(nc(C)n2C)c(Cl)c1-c1cccn2c(C(=O)c3cc(F)c(N)c(F)c3)cc(C)c12]

A mixture of (4-amino-3,5-difluorophenyl)(8-(4-chloro-1,2,6-trimethyl-1H-benzo[d]imidazol-5-yl)indolizin-3-yl)methanone (1.0 g, 2.2 mmol) and THF (10 ml) was added NIS (500 mg, 2.2 mmol) and stirred at rt for 1 hour. The resulting mixture was diluted with EtOAc, washed with water, saturated Na₂SO₃, saturated NaHCO₃, and 20% NaCl. The EtOAc solution was dried over Na₂SO₄, filtered, and concentrated. The residue was purified by column chromatography (SiO2, 0-10% MeOH in EtOAc) to give (4-amino-3,5-difluorophenyl)(8-(4-chloro-1,2,6-trimethyl-1H-benzo[d]imidazol-5-yl)-1-iodoindolizin-3-yl)methanone.

### Step 2

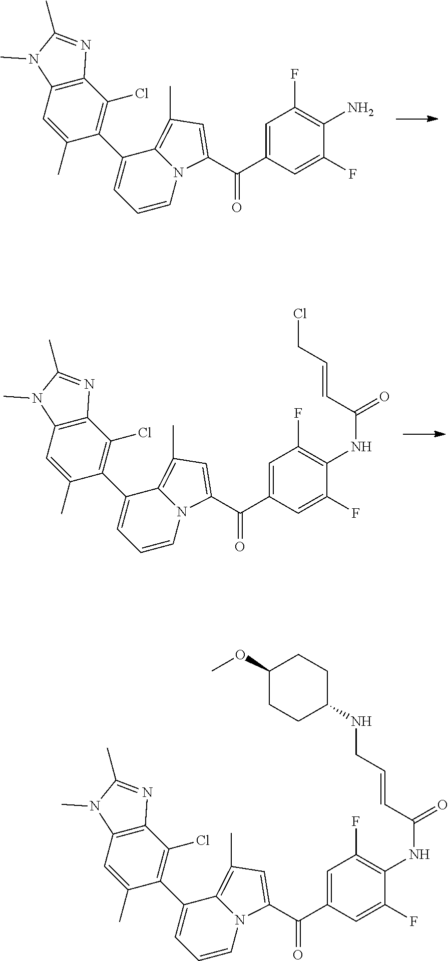 [SMILES: Cc1cc2c(nc(C)n2C)c(Cl)c1-c1cccn2c(C(=O)c3cc(F)c(N)c(F)c3)cc(C)c12 ; Cc1cc2c(nc(C)n2C)c(Cl)c1-c1cccn2c(C(=O)c3cc(F)c(NC(=O)/C=C/CCl)c(F)c3)cc(C)c12 ; CO[C@H]1CC[C@H](NC/C=C/C(=O)Nc2c(F)cc(C(=O)c3cc(C)c4c(-c5c(C)cc6c(nc(C)n6C)c5Cl)cccn34)cc2F)CC1]

A mixture of (4-amino-3,5-difluorophenyl)(8-(4-chloro-1,2,6-trimethyl-1H-benzo[d]imidazol-5-yl)-1-iodoindolizin-3-yl)methanone (43 mg, 72 μmol), (E)-4-chlorobut-2-enoic acid (18 mg, 150 μmol), triethylamine 90 μL (650 μmol) in DMA (1 mL) was added T3P (1.6 M solution in THF. 180 μL, 290 μmol) and the reaction was stirred at rt for 1.5 hours. The mixture was diluted with water and extracted 3 times with EtOAc. The EtOAc solution was dried over Na₂SO₄, filtered, and concentrated to give crude (E)-4-chloro-N-(4-(8-(4-chloro-1,2,6-trimethyl-1H-benzo[d]imidazol-5-yl)-1-iodoindolizine-3-carbonyl)-2,6-difluorophenyl)but-2-enamide.

The crude material was dissolved in DMF 0.5 ml and then 4-methoxycyclohexanamine; hydrochloride (36 mg, 220 μmol), KI (36 mg, 220 μmol), and K₃P04 (92 mg, 430 μmol) were added. The mixture was stirred at rt for 1 hour, diluted with DMSO 1 ml, and filtered. The mixture was purified by HPLC (ODS. MeCN-water. 0.1% HCO2H) and the fractions were combined, diluted with EtOAc, washed with saturated NaHCO₃ and 20% NaCl, dried over Na₂SO₄, filtered, and concentrated to give the title compound.

### Example 76

(E)-N-(4-(8-(1,2-dimethyl-6-(trifluoromethyl)-1H-benzo[d]imidazol-5-yl)indolizine-3-carbonyl)-2,6-difluorophenyl)-4-((1-fluoro-2-methylpropan-2-yl)amino)but-2-enamide

The procedure of Example 76 was performed in the same manner as Example 2 (Step 3) to give the title compound.

### Example 77

### (E)-N-(4-(8-(1,2-dimethyl-6-(trifluoromethyl)-1H-benzo[d]imidazol-5-yl)indolizine-3-carbonyl)-2,6-difluorophenyl)-4-((tetrahydrofuran-3-yl)amino)but-2-enamide

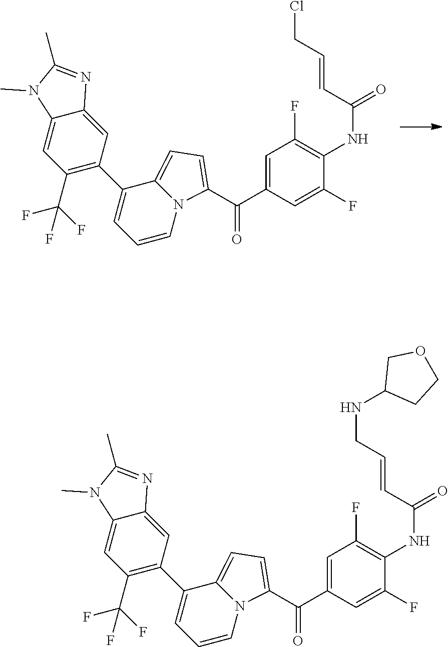 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCOC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol). 3-aminotetrahydrofuran (5.05 μL, 0.0522 mmol), and KI (12.7 mg. 0.0781 mmol) in DMSO (1.00 mL) was stirred at 35° C. for 1 hour. The resulting mixture was purified by preparative HPLC (water:acetonitrile (0.1% formic acid)) to give the title compound (14.4 mg, 0.0232 mmol).

### Example 78

(E)-N-(4-(8-(1,2-dimethyl-6-(trifluoromethyl)-1H-benzo[d]imidazol-5-yl)indolizine-3-carbonyl)-2,6-difluorophenyl)-4-((tetrahydro-2H-pyran-3-yl)amino)but-2-enamide

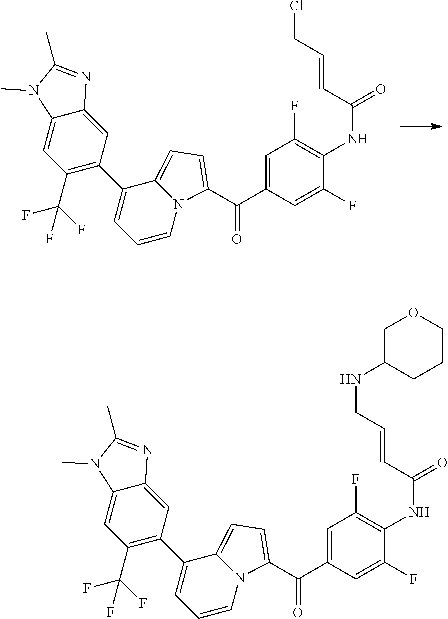 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCCOC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol), tetrahydro-2H-pyran-3amine (6.04 μL, 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.1% formic acid)) to give the title compound (13.7 mg, 0.0216 mmol).

### Example 79

(E)-4-((8-oxabicyclo[3.2.1]octan-3-yl)amino)-N-(4-(8-(1,2-dimethyl-6-(trifluoromethyl)-1H-benzo[d]imidazol-5-yl)indolizine-3-carbonyl)-2,6-difluorophenyl)but-2-enamide

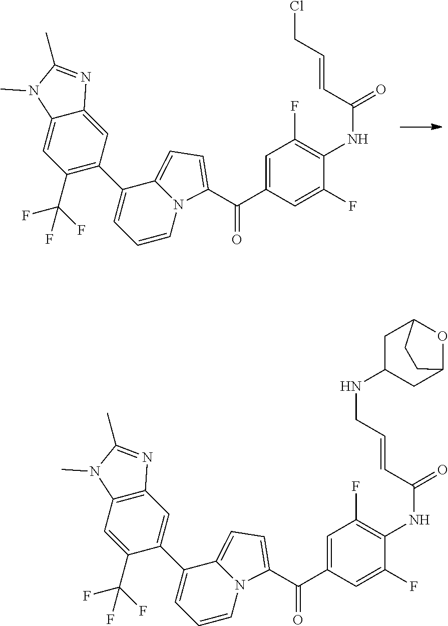 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CC7CCC(C6)O7)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol), 8-oxabicyclo[3.2.1]octan-3-amine (6.62 μL, 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.1% formic acid)) to give the title compound (13.8 mg, 0.0205 mmol).

### Example 80

### (E)-N-(4-(8-(1,2-dimethyl-6-(trifluoromethyl)-1H-benzo[d]imidazol-5-yl)indolizine-3-carbonyl)-2,6-difluorophenyl)-4-(((1r,4r)-4-fluorocyclohexyl)amino)but-2-enamide

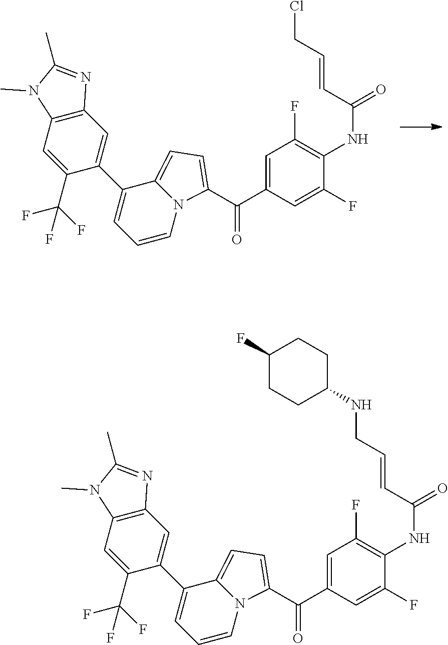 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CN[C@H]6CC[C@H](F)CC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (10 mg, 0017 mmol), trans-4-fluoro-cyclohexylamine hydrochloride (8 mg, 0.05 mmol). KI (9 mg, 0.05 mmol) and K₃PO₄ (18 mg, 0.085 mmol) in DMSO (200 μL) was stirred at 30° C. for 2 hours The reaction mixture was diluted with DMSO then filtered and purified by preparative HPLC (water:acetonitrile (0.1% formic acid)). The purified fractions were washed with sat. NaHCO₃ aq., extracted with CHCl₃/MeOH then dried over Na₂SO₄ and evaporated, thereby obtaining the title compound (5.31 mg, 47%).

### Example 81

(E)-N-(4-(8-(1,2-dimethyl-6-(trifluoromethyl)-1H-benzo[d]imidazol-5-indolizine-3-carbonyl)-2,6-difluorophenyl)-4-(((1s,4s)-4-fluorocyclohexyl)amino)but-2-enamide

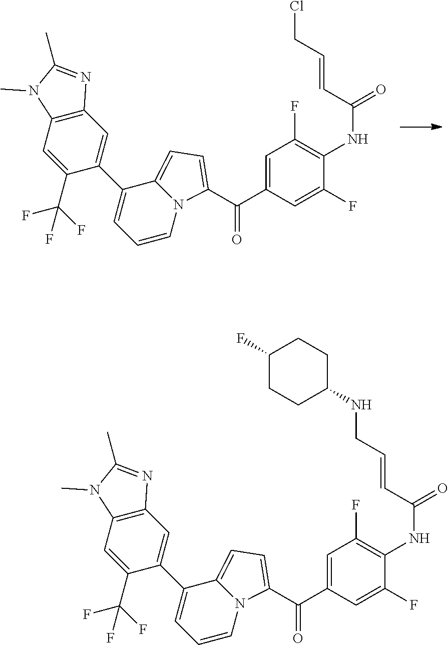 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CN[C@H]6CC[C@@H](F)CC6)c(F)c5)ccc34)c(C(F)(F)F)cc2n1C]

The procedure of Example 80 was performed except that cis-4-fluoro-cyclohexylamine hydrochloride (8 mg, 0.05 mmol) was used instead of trans-4-fluoro-cyclohexylamine hydrochloride in Example 80, thereby obtaining the title compound (5.44 mg).

### Example 82

### (E)-4-(tert-butylamino)-N-(4-(8-(4-chloro-1,6-dimethyl-2-(2,2,2-trifluoroethyl)-1H-benzo[d]imidazol-5-yl)indolizine-3-carbonyl)-2,6-difluorophenyl)but-2-enamide

The procedure of Example 82 was performed in the same manner as Example 74 to give the title compound.

### Example 83-1 and 83-2

### (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-(((1r,4r)-4-methoxycyclohexyl)amino)but-2-enamide

### Step 1

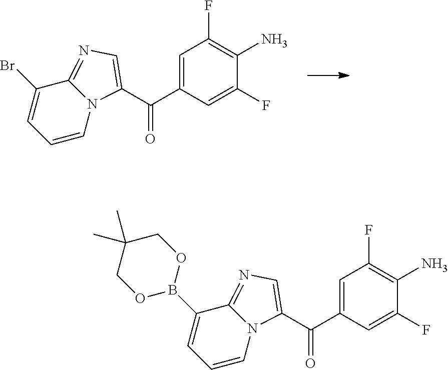 [SMILES: Nc1c(F)cc(C(=O)c2cnc3c(Br)cccn23)cc1F ; CC1(C)COB(c2cccn3c(C(=O)c4cc(F)c(N)c(F)c4)cnc23)OC1]

A solution of (4-amino-3,5-difluorophenyl)(8-bromoimidazo[1,2-a]pyridin-3-yl)methanone (200 mg). 5,5,5′,5′-tetramethyl-2,2′-bi(1,3,2-dioxaborinane) (167 mg), 1,1′-bis(diphenylphosphino)ferrocene]dichloropalladium(II) complex with dichloromethane (23.2 mg) and potassium acetate (167 mg) in DMSO (4.0 mL) was stirred at 80° C. for 20 hours. After dilution with ethyl acetate, the mixture was washed with water and a saturated sodium chloride solution, followed by drying over sodium sulfate. The solvent was evaporated under reduced pressure, thereby obtaining crude (4-amino-3,5-difluorophenyl)(8-(5,5-dimethyl-1,3,2-dioxaborinan-2-yl)imidazo[1,2-a]pyridin-3-yl)methanone (223 mg).

### Step 2

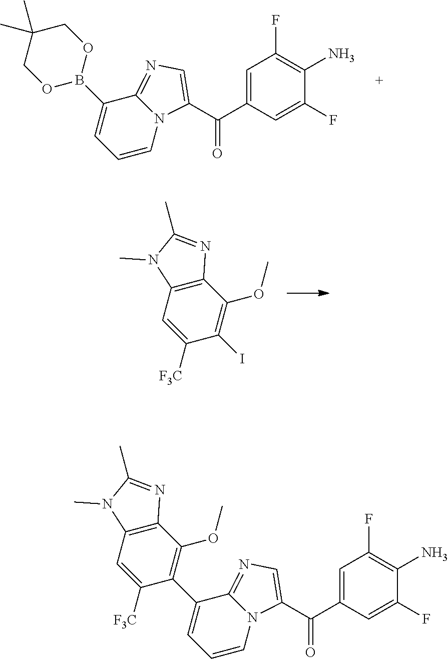

A solution of crude (4-amino-3,5-difluorophenyl)(8-(5,5-dimethyl-1,3,2-dioxaborinan-2-yl)imidazo[1,2-a]pyridin-3-yl)methanone (120 mg) obtained in step 1, 5-iodo-4-methoxy-1,2-dimethyl-6-(trifluoromethyl)-1H-benzo[d]imidazole (100 mg), (2-dicyclohexylphosphino-2′,6′-dimethoxybiphenyl) [2-(2′-amino-1,1′-biphenyl)]palladium(II) methanesulfonate (19.1 mg) and 0.5 M tripotassium phosphate solution in water (1.63 mL) in tetrahydrofuran (10.0 mL) was stirred at 80° C. for 1 hour. After dilution with ethyl acetate, the mixture was washed with water and a saturated sodium chloride solution, followed by drying over sodium sulfate. After evaporation, the residue was purified by column chromatography (chloroform:ethanol), thereby obtaining (4-amino-3,5-difluorophenyl)(8-(4-methoxy-1,2-dimethyl-6-(trifluoromethyl)-1H-benzo[d]imidazol-5-yl)imidazo[1,2-a]pyridin-3-yl)methanone (66 mg).

### Step 3, 4

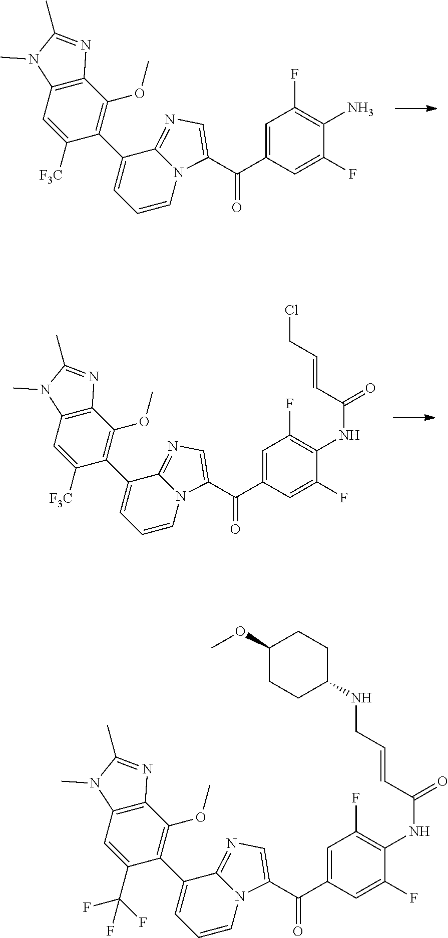 [SMILES: COc1c(-c2cccn3c(C(=O)c4cc(F)c(N)c(F)c4)cnc23)c(C(F)(F)F)cc2c1nc(C)n2C ; COc1c(-c2cccn3c(C(=O)c4cc(F)c(NC(=O)/C=C/CCl)c(F)c4)cnc23)c(C(F)(F)F)cc2c1nc(C)n2C ; COc1c(-c2cccn3c(C(=O)c4cc(F)c(NC(=O)/C=C/CN[C@H]5CC[C@H](OC)CC5)c(F)c4)cnc23)c(C(F)(F)F)cc2c1nc(C)n2C]

The procedure of Example 10 (steps 7 and 8) was performed except that (4-amino-3,5-difluorophenyl)(8-(4-methoxy-1,2-dimethyl-6-(trifluoromethyl)-1H-benzo[d]imidazol-5-yl)imidazo[1,2-a]pyridin-3-yl)methanone (66 mg) obtained in step 2 was used instead of (4-amino-3,5-difluorophenyl)(8-(4-chloro-6-ethyl-1-methyl-1H-benzo[d]imidazol-5-yl)indolizin-3-yl)methanone used in Example 10 (step 7), thereby obtaining title compound (34.0 mg).

### Step 5

(E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-(((1r,4r)-4-methoxycyclohexyl)amino)but-2-enamide (22.0 mg) obtained in step 4 was dissolved in EtOH and purified by preparative HPLC (CHIRALPAK IC, hexane-ethanol (0.1% triethyl amine)) to afford (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-(((1r,4r)-4-methoxycyclohexyl)amino)but-2-enamide (Example 83-1, 8.20 mg, single isomer. 1^{st}-eluting isomer) and (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-(((1r,4r)-4-methoxycyclohexyl)amino)but-2-enamide (Example 83-2, 8.10 mg, single isomer, 2^{nd}-eluting isomer).

### Example 84

### (E)-N-(4-(8-(1,2-dimethyl-6-(trifluoromethyl)-1H-benzo[d]imidazol-5-yl)indolizine-3-carbonyl)-2,6-difluorophenyl)-4-((4-fluorobicyclo[2.2.1]heptan-1-yl)amino)but-2-enamide

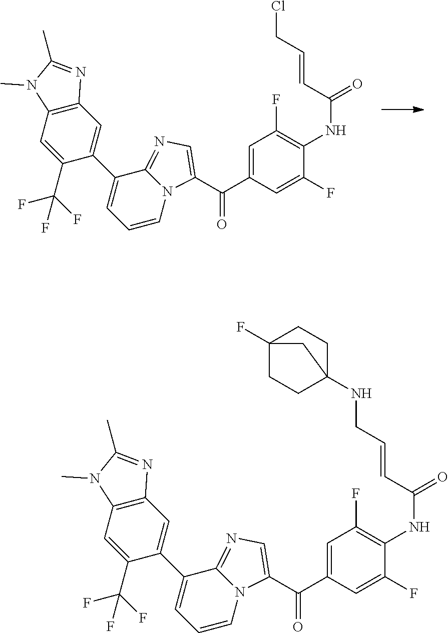 [SMILES: Cc1nc2cc(-c3cccn4c(C(=O)c5cc(F)c(NC(=O)/C=C/CCl)c(F)c5)cnc34)c(C(F)(F)F)cc2n1C ; Cc1nc2cc(-c3cccn4c(C(=O)c5cc(F)c(NC(=O)/C=C/CNC67CCC(F)(CC6)C7)c(F)c5)cnc34)c(C(F)(F)F)cc2n1C]

The procedure of Example 80 was performed except that 4-fluorobicyclo[2.2.1]heptan-1-amine hydrochloride (8 mg, 0.05 mmol) was used instead of trans-4-fluoro-cyclohexylamine hydrochloride in Example 80, thereby obtaining the title compound (3.14 mg).

### Example 85

(E)-N-(4-(8-(1,2-dimethyl-6-(trifluoromethyl)-1H-benzo[d]imidazol-5-yl)indolizine-3-carbonyl)-2,6-difluorophenyl)-4-((4-fluorobicyclo[2.2.2]octan-1-yl)amino)but-2-enamide

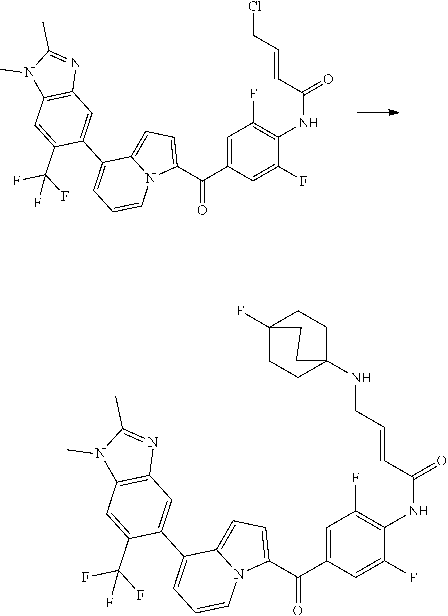 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC67CCC(F)(CC6)CC7)c(F)c5)ccc34)c(C(F)(F)F)cc2n1C]

The procedure of Example 80 was performed except that 4-fluorobicyclo[2.2.2]octan-1-amine hydrochloride (9 mg, 0.05 mmol) was used instead of trans-4-fluoro-cyclohexylamine hydrochloride in Example 80, thereby obtaining the title compound (5.37 mg).

### Example 86

### (E)-4-(tert-butylamino)-N-(4-(8-(4-chloro-2-(methoxymethyl)-1,6-dimethyl-1H-benzo[d]imidazol-5-yl)indolizine-3-carbonyl)-2,6-difluorophenyl)but-2-enamide

The procedure of Example 86 was performed in the same manner as Example 74 to give the title compound.

### Example 87

(E)-N-(4-(8-(4-chloro-1,2,6-trimethyl-1H-benzo[d]imidazol-5-yl)-1-methylindolizine-3-carbonyl)-2,6-difluorophenyl)-4-(((1r,4r)-4-methoxycyclohexyl)amino)but-2-enamide

### Step 1

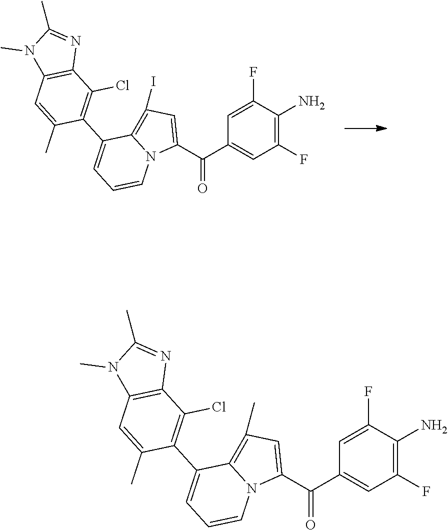 [SMILES: Cc1cc2c(nc(C)n2C)c(Cl)c1-c1cccn2c(C(=O)c3cc(F)c(N)c(F)c3)cc(I)c12 ; Cc1cc2c(nc(C)n2C)c(Cl)c1-c1cccn2c(C(=O)c3cc(F)c(N)c(F)c3)cc(C)c12]

Trimethylboroxyn (0.042 mL), tetrakistripheniylphosphineparaddium (15.6 mg), potassium carbonate (74.9 mg) was added to (4-amino-3,5-difluorophenyl)(8-(4-chloro-1,2,6-trimethyl-1H-benzo[d]imidazol-5-yl)-1-iodoindolizin-3-yl)methanone (80.0 mg) in 1,4-dioxane (1.0 mL). After stirring at 100° C. for 1 hour, the reaction mixture was diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-methylindolizin-3-yl)methanone (66.0 mg).

### Step 2

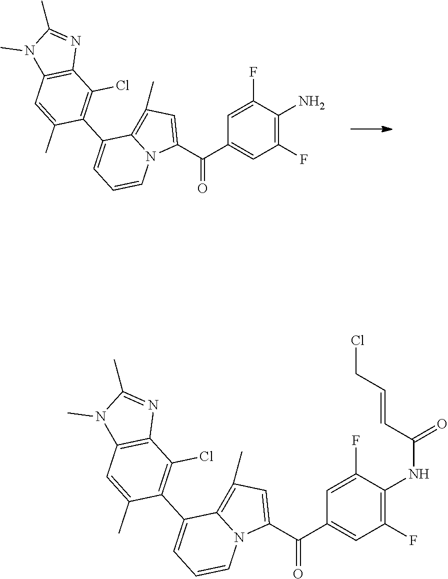 [SMILES: Cc1cc2c(nc(C)n2C)c(Cl)c1-c1cccn2c(C(=O)c3cc(F)c(N)c(F)c3)cc(C)c12 ; Cc1cc2c(nc(C)n2C)c(Cl)c1-c1cccn2c(C(=O)c3cc(F)c(NC(=O)/C=C/CCl)c(F)c3)cc(C)c12]

(E)-4-chlorobut-2-enoic acid (16.4 mg) and T3P (86.4 mg) in THF (0.17 mL) was added to a solution of (4-amino-3,5-difluorophenyl)(8-(4-chloro-1,2,6-trimethyl-1H-benzo[d]imidazol-5-yl)-1-methylindolizin-3-yl)methanone (66 mg) in DMF (1.0 mL), then triethylamine (0.19 mL) was added to the mixture. After stirring at room temperature for 30 min. (E)-4-chlorobut-2-enoic acid (16.4 mg) and T3P (86.4 mg) in THF (0.17 mL) was added to the mixture. After stirring at room temperature for another 30 min. the reaction mixture was diluted with EtOAc and water, extracted with EtOAc. The separated organic phase was washed with brine and dried over sodium sulfate, filtered, concentrated, thereby obtaining (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide (91.4 mg) as a crude product.

### Step 3

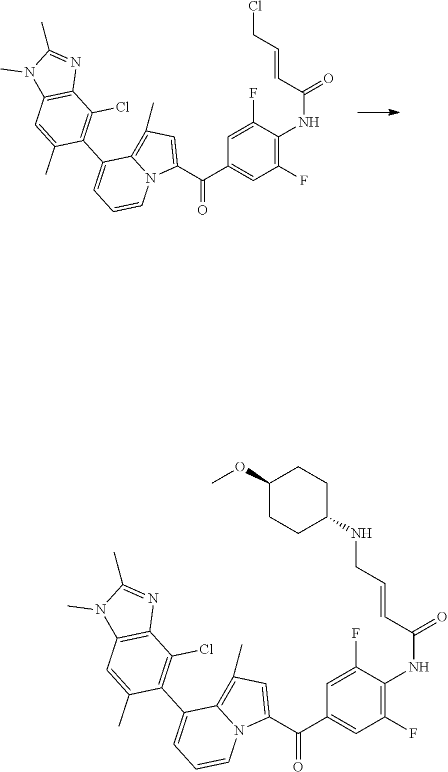 [SMILES: Cc1cc2c(nc(C)n2C)c(Cl)c1-c1cccn2c(C(=O)c3cc(F)c(NC(=O)/C=C/CCl)c(F)c3)cc(C)c12 ; CO[C@H]1CC[C@H](NC/C=C/C(=O)Nc2c(F)cc(C(=O)c3cc(C)c4c(-c5c(C)cc6c(nc(C)n6C)c5Cl)cccn34)cc2F)CC1]

Potassium iodide (173 mg), potassium phosphate tribasic (67.7 mg), 4-methoxycyclohexanamine hydrochloride (67.6 mg) was added to (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide (91.5 mg) in DMF (1 mL). After stirring at 40° C. for 1.5 hours, the reaction mixture was diluted with EtOAc and water, extracted with EtOAc. The separated organic phase was washed with brine, dried over sodium sulfate, filtered, concentrated. The residue was purified by preparative reversed-phase HPLC (water:acetonitrile (0.10% formic acid)). Saturated sodium bicarbonate water was added to the purified fractions containing the title compound, followed by extraction with ethyl acetate. The extract was washed with brine and dried over sodium sulfate. The solvent was evaporated under reduced pressure, thereby obtaining the title compound (34.0 mg).

### Example 88

### (E)-N-(4-(8(4-chloro-1,2,6-trimethyl-1H-benzo[d]imidazol-5-yl)-1-vinylindolizine-3-carbonyl)-2,6-difluorophenyl)-4-(((1r,4r)-4-methoxycyclohexyl)amino)but-2-enamide

### Step 1

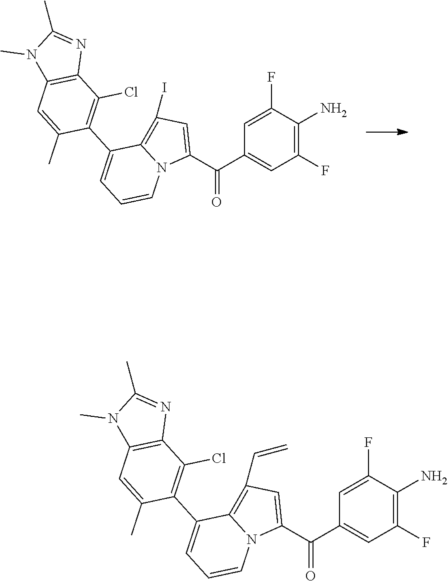 [SMILES: Cc1cc2c(nc(C)n2C)c(Cl)c1-c1cccn2c(C(=O)c3cc(F)c(N)c(F)c3)cc(I)c12 ; C=Cc1cc(C(=O)c2cc(F)c(N)c(F)c2)n2cccc(-c3c(C)cc4c(nc(C)n4C)c3Cl)c12]

Potassium trifluoro(vinyl)boranuide (36.3 mg). [1,1′-Bis(diphenylphosphino)ferrocene]dichloropalladium(II), complex with dichloromethane (11.1 mg), sodium carbonate (a 2.0 M aqueous solution, 0.22 mL) was added to (4-amino-3,5-difluorophenyl)(8-(4-chloro-1,2,6-trimethyl-1H-benzo[d]imidazol-5-yl)-1-iodoindolizin-3-yl)methanone (80.0 mg) in 1,4-dioxane (1.0 mL). After stirring at 100° C. for 4 hours, the reaction mixture was diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-vinylindolizin-3-yl)methanone (61.3 mg).

### Step 2

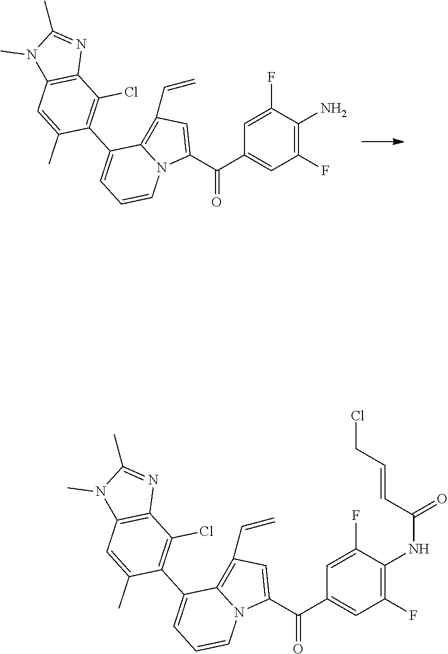 [SMILES: C=Cc1cc(C(=O)c2cc(F)c(N)c(F)c2)n2cccc(-c3c(C)cc4c(nc(C)n4C)c3Cl)c12 ; C=Cc1cc(C(=O)c2cc(F)c(NC(=O)/C=C/CCl)c(F)c2)n2cccc(-c3c(C)cc4c(nc(C)n4C)c3Cl)c12]

The procedure of Example 87 (step 2) was performed except that (4-amino-3,5-difluorophenyl)(8-(4-chloro-1,2,6-trimethyl-1H-benzo[d]imidazol-5-yl)-1-vinylindolizin-3-yl)methanone (20.0 mg) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-vinylindolizine-3-carbonyl)-2,6-difluorophenyl)but-2-enamide (29.6 mg) as a crude product.

### Step 3

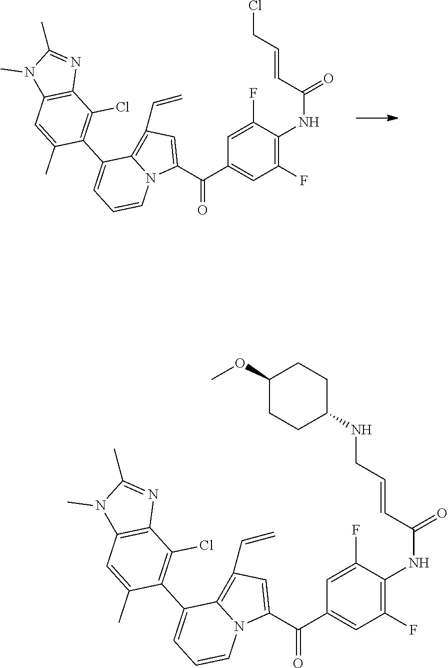 [SMILES: C=Cc1cc(C(=O)c2cc(F)c(NC(=O)/C=C/CCl)c(F)c2)n2cccc(-c3c(C)cc4c(nc(C)n4C)c3Cl)c12 ; C=Cc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12]

The procedure of Example 87 (step 3) was performed except that ((E)-4-chloro-N-(4-(8-(4-chloro-1,2,6-trimethyl-1H-benzo[d]imidazol-5-yl)-1-vinylindolizine-3-carbonyl)-2,6-difluorophenyl)but-2-enamide (29.6 mg) was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound (8.5 mg).

### Example 89

(E)-N-(4-(8-(4-chloro-1,2,6-trimethyl-1H-benzo[d]imidazol-5-yl)-1-ethylindolizine-3-carbonyl)-2,6-difluorophenyl)-4-(((1r,4r)-4-methoxycyclohexyl)amino)but-2-enamide

### Step 1

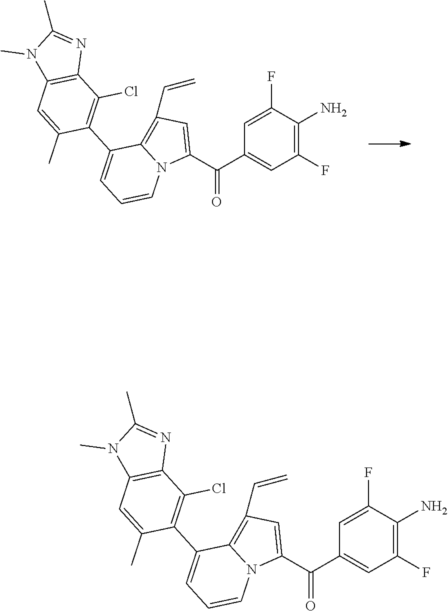 [SMILES: C=Cc1cc(C(=O)c2cc(F)c(N)c(F)c2)n2cccc(-c3c(C)cc4c(nc(C)n4C)c3Cl)c12 ; C=Cc1cc(C(=O)c2cc(F)c(N)c(F)c2)n2cccc(-c3c(C)cc4c(nc(C)n4C)c3Cl)c12]

Palladium on carbon (20.0 mg) was added to (4-amino-3,5-difluorophenyl)(8-(4-chloro-1,2,6-trimethyl-1H-benzo[d]imidazol-5-yl)-1-vinylindolizin-3-yl)methanone (19.6 mg) in ethanol (1.0 mL). After stirring at room temperature for 2 hours, the reaction mixture was filtered through a Celite pad, washed with methanol and organic solvents are evaporated under reduced pressure. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-ethylindolizin-3-yl)methanone (15.2 mg).

### Step 2

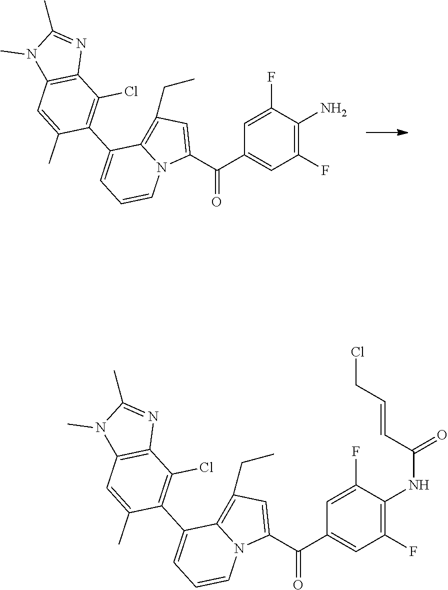 [SMILES: CCc1cc(C(=O)c2cc(F)c(N)c(F)c2)n2cccc(-c3c(C)cc4c(nc(C)n4C)c3Cl)c12 ; CCc1cc(C(=O)c2cc(F)c(NC(=O)/C=C/CCl)c(F)c2)n2cccc(-c3c(C)cc4c(nc(C)n4C)c3Cl)c12]

The procedure of Example 87 (step 2) was performed except that (4-amino-3,5-difluorophenyl)(8-(4-chloro-1,2,6-trimethyl-1H-benzo[d]imidazol-5-yl)-1-ethylindolizin-3-yl)methanone (15.2 mg) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-ethylindolizine-3-carbonyl)-2,6-difluorophenyl)but-2-enamide (22.8 mg) as a crude product.

### Step 3

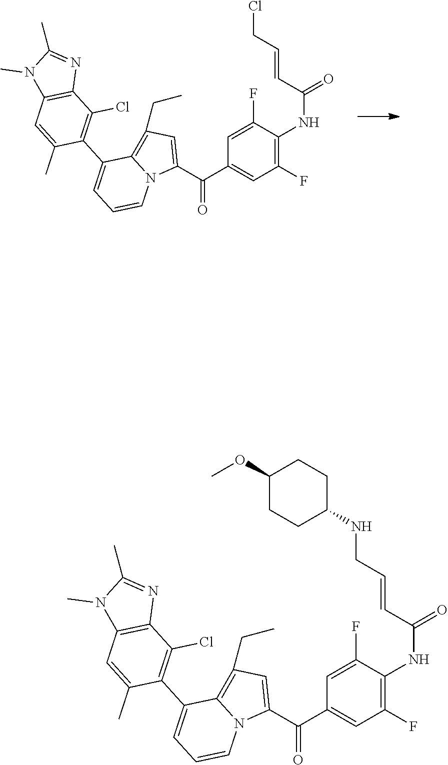 [SMILES: CCc1cc(C(=O)c2cc(F)c(NC(=O)/C=C/CCl)c(F)c2)n2cccc(-c3c(C)cc4c(nc(C)n4C)c3Cl)c12 ; CCc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12]

The procedure of Example 87 (step 3) was performed except that (E)-4-chloro-N-(4-(8-(4-chloro-1,2,6-trimethyl-1H-benzo[d]imidazol-5-yl)-1-ethylindolizine-3-carbonyl)-2,6-difluorophenyl)but-2-enamide (22.8 mg) was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound (9.3 mg).

### Example 90

### (E)-N-(4-(8-(1,2-dimethyl-6-(trifluoromethyl)-1H-benzo[d]imidazol-5-yl)indolizine-3-carbonyl-2,6-difluorophenyl)-4-(((1R,3R)-3-fluorocyclohexyl)amino)but-2-enamide and (E)-N-(4-(8-(1,2-dimethyl-6-(trifluoromethyl)-1H-benzo[d]imidazol-5-yl)indolizine-3-carbonyl)-2,6-difluorophenyl)-4-(((1S,3S)-3-fluorocyclohexyl)amino)but-2-enamide

The procedure of Example 90 was performed in the same manner as Example 2 (Step 3) to give the title compound.

### Example 91

### (R,E)-N-(4-(8-(1,2-dimethyl-6-(trifluoromethyl)-1H-benzo[d]imidazol-5-yl)indolizine-3-carbonyl)-2,6-difluorophenyl)-4-(tetrahydrofuran-3-yl)amino)but-2-enamide

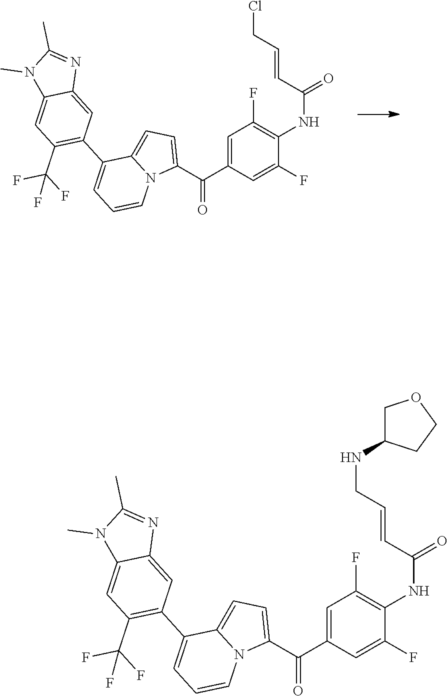 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CN[C@@H]6CCOC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol). (3R)-tetrahydrofuran-3-amine (5.04 μL. 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.1% formic acid)) to give the title compound (14.3 mg, 0.0231 mmol).

### Example 92

### (S,E)-N-(4-(8-(1,2-dimethyl-6-(trifluoromethyl-1H-benzo[d]imidazol-5-yl)indolizine-3-carbonyl)-2,6-difluorophenyl)-4-((tetrahydrofuran-3-yl)amino)but-2-enamide

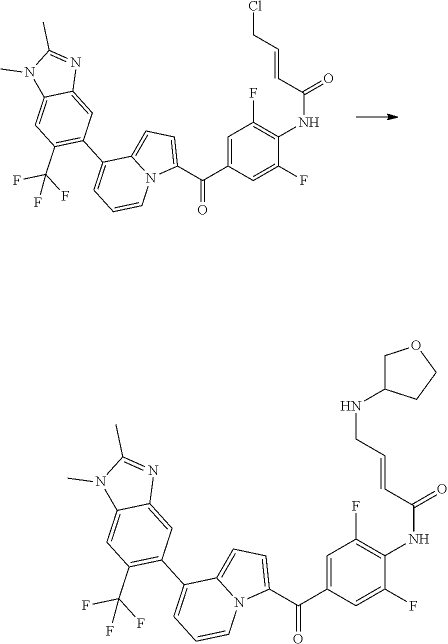 [SMILES: Cc1nc2cc(-c3cccn4c(C(=O)c5cc(F)c(NC(=O)/C=C/CCl)c(F)c5)ccc34)c(C(F)(F)F)cc2n1C ; Cc1nc2cc(-c3cccn4c(C(=O)c5cc(F)c(NC(=O)/C=C/CNC6CCOC6)c(F)c5)ccc34)c(C(F)(F)F)cc2n1C]

A solution of (E)-4-chloro-N-(4-(8-(1,2-dimethyl-6-(trifluoromethyl)-1H-benzo[d]imidazol-5-yl)indolizine-3-carbonyl)-2,6-difluorophenyl)but-2-enamide (15.0 mg, 0.0263 mmol). (3S)-tetrahydrofuran-3-amine (5.04 μL, 0.0522 mmol), and KI (12.7 mg, 0.0781 mmol) in DMSO (1.00 mL) was stirred at 35° C. for 1 hour. The resulting mixture was purified by preparative HPLC (water:acetonitrile (0.1% formic acid)) to give the title compound (10.5 mg, 0.0162 mmol).

### Example 93

(E)-N-(4-(8-(4-chloro-1,2,6-trimethyl-1H-benzo[d]imidazol-5-yl)-1-(hydroxymethyl)indolizine-3-carbonyl-2,6-difluorophenyl)-4-(((1r,4r)-4-methoxycyclohexyl)amino)but-2-enamide

### Step 1

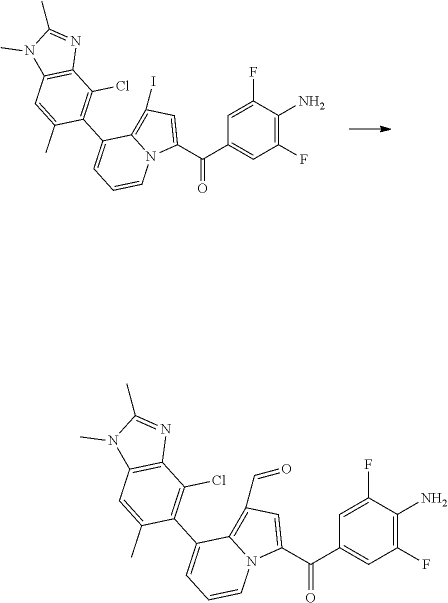 [SMILES: Cc1cc2c(nc(C)n2C)c(Cl)c1-c1cccn2c(C(=O)c3cc(F)c(N)c(F)c3)cc(I)c12 ; Cc1cc2c(nc(C)n2C)c(Cl)c1-c1cccn2c(C(=O)c3cc(F)c(N)c(F)c3)cc(C=O)c12]

2-isocyano-2-methyl-propane (0.077 mL), triethylsilane (0.065 mL), sodium carbonate (17.2 mg), diacetoxypalladium (0.9 mg) and JohnPhos (1.8 mg) was added to (4-amino-3,5-difluorophenyl)(8-(4-chloro-1,2,6-trimethyl-1H-benzo[d]imidazol-5-yl)-1-iodoindolizin-3-yl)methanone (80.0 mg) in DMF (1.0 mL). After stirring at 70° C. for 1 hour, the reaction mixture was diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining 3-(4-amino-3,5-difluorobenzoyl)-8-(4-chloro-1,2,6-trimethyl-1H-benzo[d]imidazol-5-yl)indolizine-1-carbaldehyde (77.4 mg).

### Step 2

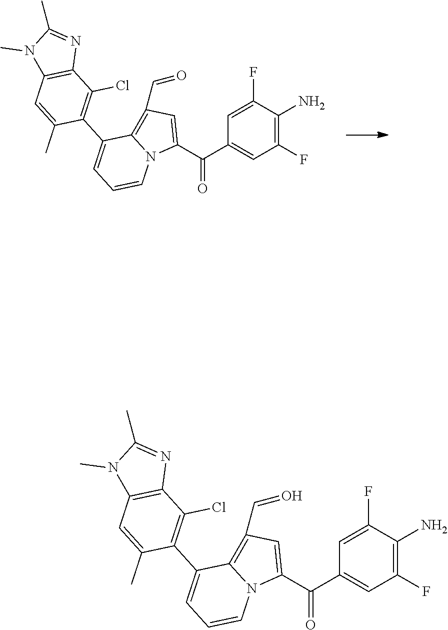 [SMILES: Cc1cc2c(nc(C)n2C)c(Cl)c1-c1cccn2c(C(=O)c3cc(F)c(N)c(F)c3)cc(C=O)c12 ; Cc1cc2c(nc(C)n2C)c(Cl)c1-c1cccn2c(C(=O)c3cc(F)c(N)c(F)c3)cc(C=O)c12]

Sodium triacetoxyborohydride (34.3 mg) was added to 3-(4-amino-3,5-difluorobenzoyl)-8-(4-chloro-1,2,6-trimethyl-1H-benzo[d]imidazol-5-yl)indolizine-1-carbaldehyde (66.5 mg) in THF (2.0 mL). After stirring at room temperature overnight, the solvent was removed under reduced pressure. The residue was diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-(hydroxymethyl)indolizin-3-yl)methanone (51.9 mg).

### Step 3

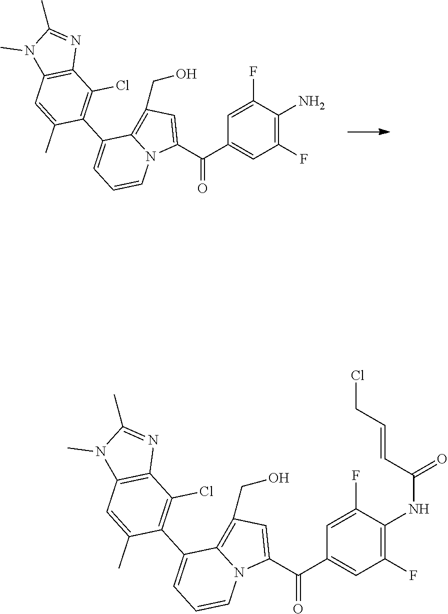 [SMILES: Cc1cc2c(nc(C)n2C)c(Cl)c1-c1cccn2c(C(=O)c3cc(F)c(N)c(F)c3)cc(CO)c12 ; Cc1cc2c(nc(C)n2C)c(Cl)c1-c1cccn2c(C(=O)c3cc(F)c(NC(=O)/C=C/CCl)c(F)c3)cc(CO)c12]

The procedure of Example 87 (step 2) was performed except that (4-amino-3,5-difluorophenyl)(8-(4-chloro-1,2,6-trimethyl-1H-benzo[d]imidazol-5-yl)-1-(hydroxymethyl)indolizin-3-yl)methanone (51.9 mg) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-(hydroxymethyl)indolizine-3-carbonyl)-2,6-difluorophenyl)but-2-enamide (64.5 mg) as a crude product.

### Step 4

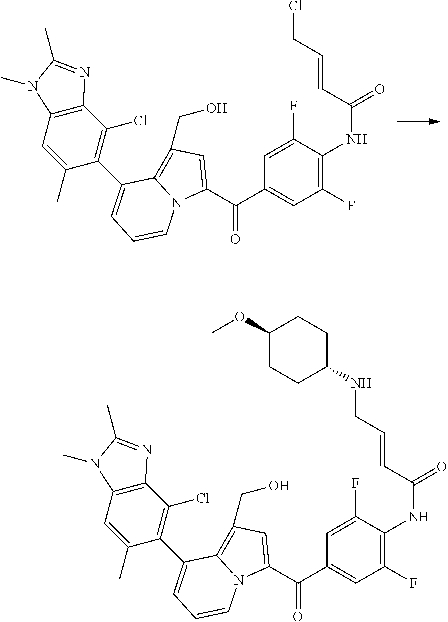 [SMILES: Cc1cc2c(nc(C)n2C)c(Cl)c1-c1cccn2c(C(=O)c3cc(F)c(NC(=O)/C=C/CCl)c(F)c3)cc(CO)c12 ; CO[C@H]1CC[C@H](NC/C=C/C(=O)Nc2c(F)cc(C(=O)c3cc(CO)c4c(-c5c(C)cc6c(nc(C)n6C)c5Cl)cccn34)cc2F)CC1]

The procedure of Example 87 (step 3) was performed except that (E)-4-chloro-N-(4-(8-(4-chloro-1,2,6-trimethyl-1H-benzo[d]imidazol-5-yl)-1-(hydroxymethyl)indolizine-3-carbonyl)-2,6-difluorophenyl)but-2-enamide (62.7 mg) was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound (9.2 mg).

### Example 94

### (E)-N-(4-(8-(1,2-dimethyl-6-(trifluoromethyl)-1H-benzo[d]imidazol-5-yl)indolizine-3-carbonyl)-2,6-difluorophenyl)-4-(((1R,3S)-3-fluorocyclohexyl)amino)but-2-enamide and (E)-N-(4-(8-(1,2-dimethyl-6-(trifluoromethyl)-1H-benzo[d]imidazol-5-yl)indolizine-3-carbonyl)-2,6-difluorophenyl-4-(((1S,3R)-3-fluorocyclohexyl)amino)but-2-enamide

The procedure of Example 94 was performed in the same manner as Example 2 (Step 3) to give the title compound.

### Example 95

### (E)-N-(4-(2-cyclopropyl-8-(1,2-dimethyl-6-(trifluoromethyl)-1H-benzo[d]imidazol-5-yl)imidazo[1,2-a]pyridine-3-carbonyl)-2,6-difluorophenyl)-4-(((1r,4r)-4-methoxycyclohexyl)amino)but-2-enamide

### Step 1, 2

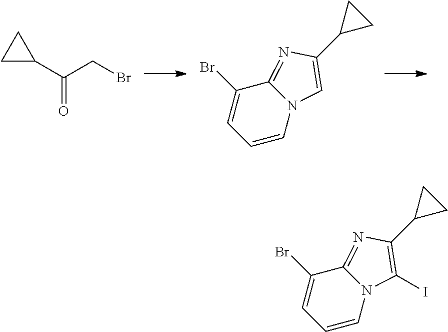 [SMILES: O=C(CBr)C1CC1 ; Brc1cccn2cc(C3CC3)nc12 ; Brc1cccn2c(I)c(C3CC3)nc12]

The procedures of Example 51 (steps 1, 2) were performed except that 2-bromo-1-cyclopropylethan-1-one (5.0 g) was used instead of 1-bromopropan-2-one in step 1 (Example 51), thereby obtaining 8-bromo-2-cyclopropyl-3-iodoimidazo[1,2-a]pyridine (6.0 g).

### Step 3

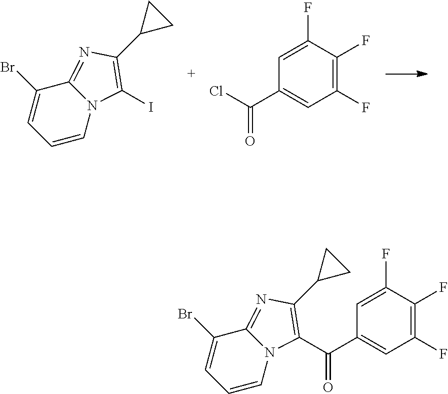 [SMILES: Brc1cccn2c(I)c(C3CC3)nc12 ; O=C(Cl)c1cc(F)c(F)c(F)c1 ; O=C(c1cc(F)c(F)c(F)c1)c1c(C2CC2)nc2c(Br)cccn12]

A solution of crude 8-bromo-2-cyclopropyl-3-iodoimidazo[1,2-a]pyridine (310 mg) obtained in step 2 in tetrahydrofuran (4 mL) was cooled to −10° C. To the reaction mixture was added isopropylmagnesium chloride-lithium chloride complex (tetrahydrofuran solution (abt. 14%), 0.723 mL). After stirring at −10° C. for 1 hour. 3,4,5-trifluorobenzoyl chloride (163 mg) was added to the reaction mixture. A reaction was performed at −10° C. for 1 hour. After warming up to room temperature, the reaction mixture was quenched with a saturated ammonium chloride solution. After the mixture was extracted with ethyl acetate, the organic layer was washed with a saturated sodium chloride solution, followed by drying over sodium sulfate. After evaporation, the obtained residue was purified by column chromatography (hexane:ethyl acetate), thereby obtaining (8-bromo-2-cyclopropylimidazo[1,2-a]pyridin-3-yl)(3,4,5-trifluorophenyl)methanone (200 mg).

### Step 4

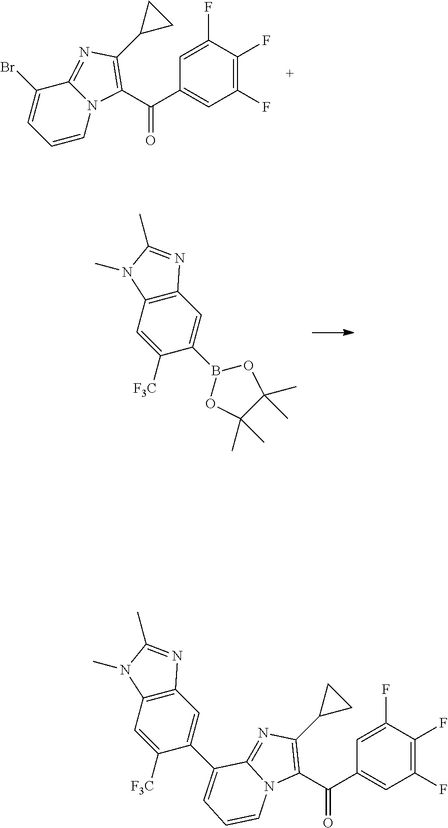

A solution of (8-bromo-2-cyclopropylimidazo[1,2-a]pyridin-3-yl)(3,4,5-trifluorophenyl)methanone (100 mg) obtained in step 3, 1,2-dimethyl-5-(4,4,5,5-tetramethyl-1,3,2-dioxaborolan-2-yl)-6-(trifluoromethyl)-1H-benzo[d]imidazole (110 mg), tetrakis(triphenylphosphine)palladium(0) (29.2 mg). 2.0 M sodium bicarbonate in water (253 IL) in 1,2-dimethoxyethane (1.5 mL) was stirred at 140° C. for 40 minutes using microwave irradiation. After dilution with ethyl acetate and water, the mixture was extracted with ethyl acetate. The combined organic layer was washed with water and a saturated sodium chloride solution, followed by drying over sodium sulfate. After evaporation, the obtained residue was purified by column chromatography (chloroform:ethanol), thereby obtaining (2-cyclopropyl-8-(1,2-dimethyl-6-(trifluoromethyl)-1H-benzo[d]imidazol-5-yl)imidazo[1,2-a]pyridin-3-yl)(3,4,5-trifluorophenyl)methanone (128 mg).

### Step 5

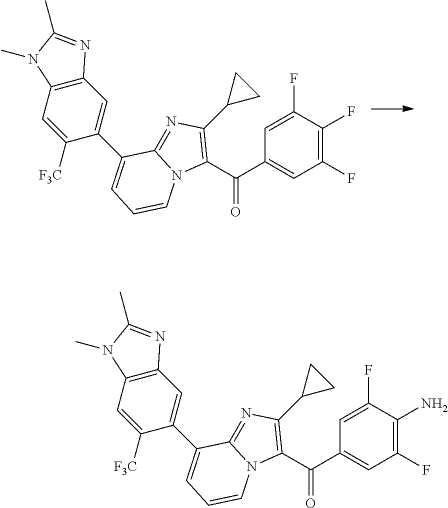 [SMILES: Cc1nc2cc(-c3cccn4c(C(=O)c5cc(F)c(F)c(F)c5)c(C5CC5)nc34)c(C(F)(F)F)cc2n1C ; Cc1nc2cc(-c3cccn4c(C(=O)c5cc(F)c(N)c(F)c5)c(C5CC5)nc34)c(C(F)(F)F)cc2n1C]

A solution of (2-cyclopropyl-8-(1,2-dimethyl-6-(trifluoromethyl)-1H-benzo[d]imidazol-5-yl)imidazo[1,2-a]pyridin-3-yl)(3,4,5-trifluorophenyl)methanone (130 mg) obtained in step 4 and 28% ammonia solution (2 mL) in 1,4-dioxane (2 mL) was stirred at 140° C. for 8 hours using microwave irradiation. After dilution with ethyl acetate and water, the mixture was extracted with ethyl acetate. The combined organic layer was washed with a saturated sodium chloride solution, followed by drying over sodium sulfate. After evaporation, the obtained residue was purified by column chromatography (hexane:ethyl acetate), thereby obtaining (4-amino-3,5-difluorophenyl)(2-cyclopropyl-8-(1,2-dimethyl-6-(trifluoromethyl)-1H-benzo[d]imidazol-5-yl)imidazo[1,2-a]pyridin-3-yl)methanone (101 mg).

### Step 6, 7

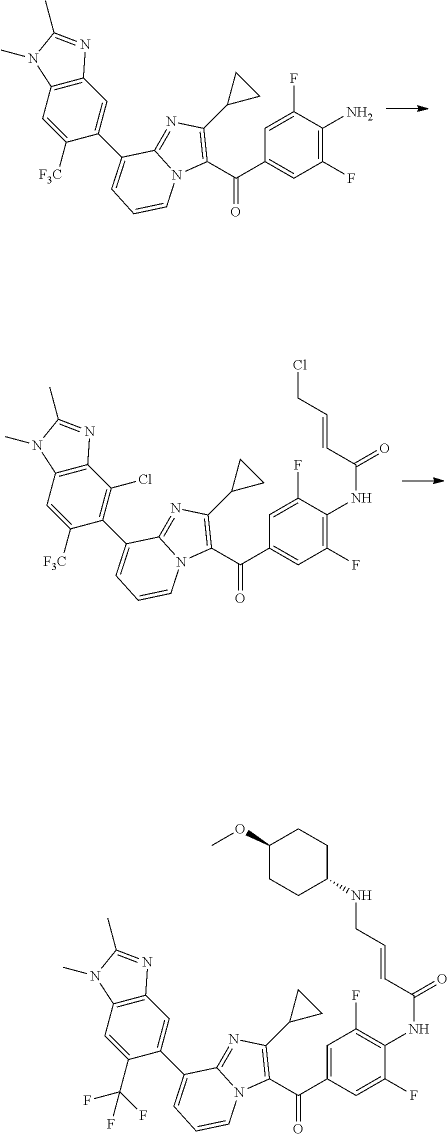

The procedures of Example 51 (steps 7, 8) were performed except that (4-amino-3,5-difluorophenyl)(2-cyclopropyl-8-(1,2-dimethyl-6-(trifluoromethyl)-1H-benzo[d]imidazol-5-yl)imidazo[1,2-a]pyridin-3-yl)methanone (101 mg) obtained in step 5 was used instead of (4-amino-3,5-difluorophenyl)(8-(1,2-dimethyl-6-(trifluoromethyl)-1H-benzo[d]imidazol-5-yl)-2-methylimidazo[1,2-a]pyridin-3-yl)methanone in Example 51 (step 7), thereby obtaining the title compound (94.7 mg).

### Example 96

### (E)-N-(4-(8-(4-chloro-1,2,6-trimethyl-1H-benzo[d]imidazol-5-yl)-1-((E)-prop-1-en-1-yl)indolizine-3-carbonyl)-2,6-difluorophenyl)-4-(((1r,4r)-4-methoxycyclohexyl)amino)but-2-enamide

The title compound (9.9 mg) was obtained using the same procedure as that of Example 88.

### Example 97

### (E)-N-(4-(8-(4-chloro-1,2,6-trimethyl-1H-benzo[d]imidazol-5-yl)-1-propylindolizine-3-carbonyl)-2,6-difluorophenyl)-4-(((1r,4r)-4-methoxycyclohexyl)amino)but-2-enamide

The title compound (14.9 mg) was obtained using the same procedure as that of Example 89.

### Example 98

### (R,E)-4-(tert-butylamino)-N-(4-(8-(4-chloro-2-(1-hydroxyethyl)-1,6-dimethyl-1H-benzo[d]imidazol-5-yl)indolizine-3-carbonyl)-2,6-difluorophenyl)but-2-enamide

The procedure of Example 98 was performed in the same manner as Example 58 to give the title compound.

### Example 99

### (E)-N-(4-(8-(1,2-dimethyl-6-(trifluoromethyl)-1H-benzo[d]imidazol-5-yl)-2-ethylimidazo[1,2-a]pyridine-3-carbonyl)-2,6-difluorophenyl)-4-(((1r,4r)-4-methoxycyclohexyl)amino)but-2-enamide

### Step 1, 2

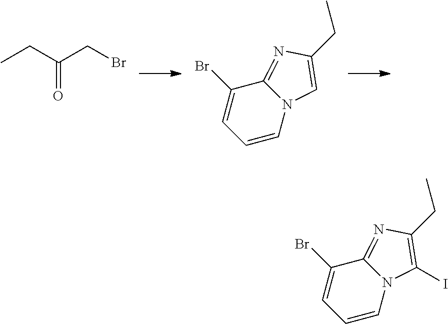

The procedures of Example 51 (steps 1, 2) were performed except that 1-bromobutan-2-one (4.6 g) was used instead of 1-bromopropan-2-one in Example 51 (step 1), thereby obtaining 8-bromo-2-ethyl-3-iodoimidazo[1,2-a]pyridine (6.5 g).

### Step 3

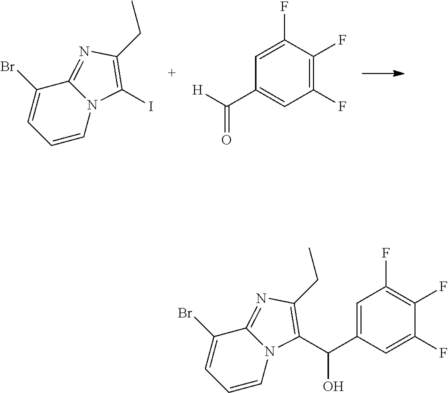 [SMILES: CCc1nc2c(Br)cccn2c1I ; O=Cc1cc(F)c(F)c(F)c1 ; CCc1nc2c(Br)cccn2c1C(O)c1cc(F)c(F)c(F)c1]

A solution of crude 8-bromo-2-ethyl-3-iodoimidazo[1,2-a]pyridine (1.60 g) obtained in step 2 in tetrahydrofuran (20 mL) was cooled to −10° C. To the reaction mixture was added isopropylmagnesium chloride-lithium chloride complex (tetrahydrofuran solution (abt. 14%). 3.86 mL). After stirring at −10° C. for 1 hour. 3,4,5-trifluorobenzaldehyde (730 mg) was added to the reaction mixture. A reaction was performed at −10° C. for 1 hour. After warming up to room temperature, the reaction mixture was quenched with a saturated ammonium chloride solution. After the mixture was extracted with ethyl acetate, the organic layer was washed with a saturated sodium chloride solution, followed by drying over sodium sulfate. After evaporation, the obtained residue was purified by column chromatography (hexane:ethyl acetate), thereby obtaining (8-bromo-2-ethylimidazo[1,2-a]pyridin-3-yl)(3,4,5-trifluorophenyl)methanol (1.50 g).

### Step 4

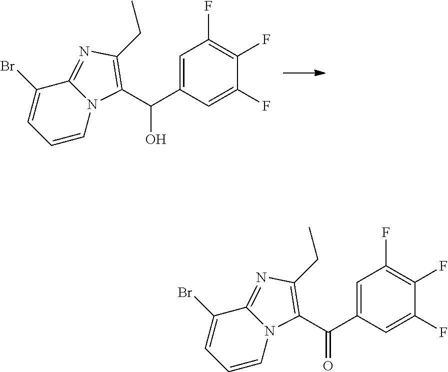 [SMILES: CCc1nc2c(Br)cccn2c1C(O)c1cc(F)c(F)c(F)c1 ; CCc1nc2c(Br)cccn2c1C(=O)c1cc(F)c(F)c(F)c1]

A solution of (8-bromo-2-ethylimidazo[1,2-a]pyridin-3-yl)(3,4,5-trifluorophenyl)methanol (1.50 g) obtained in step 3 and manganese dioxide (1.78 g) in ethyl acetate (30 mL) was stirred at 60° C. for 14 hours. The insoluble matter was filtered off, followed by evaporating the solvent under reduced pressure. The obtained residue was purified by column chromatography (hexane:ethyl acetate), thereby obtaining (8-bromo-2-ethylimidazo[1,2-a]pyridin-3-yl)(3,4,5-trifluorophenyl)methanone (1.28 g).

### Step 5, 6

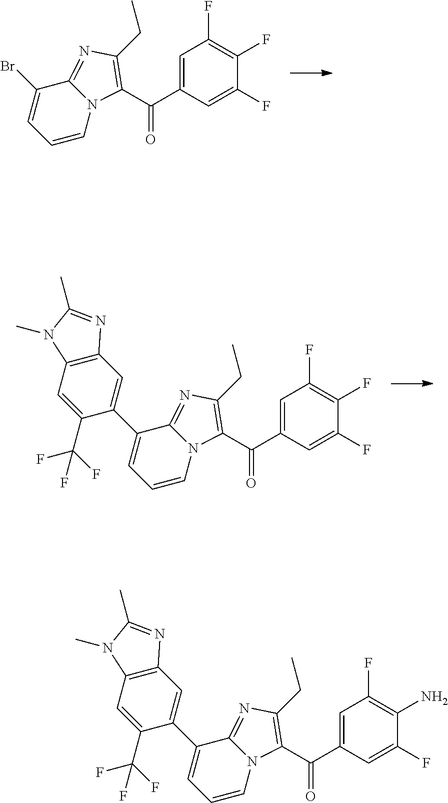 [SMILES: CCc1nc2c(Br)cccn2c1C(=O)c1cc(F)c(F)c(F)c1 ; CCc1nc2c(-c3cc4nc(C)n(C)c4cc3C(F)(F)F)cccn2c1C(=O)c1cc(F)c(F)c(F)c1 ; CCc1nc2c(-c3cc4nc(C)n(C)c4cc3C(F)(F)F)cccn2c1C(=O)c1cc(F)c(N)c(F)c1]

The procedures of Example 95 (steps 4, 5) were performed except that (8-bromo-2-ethylimidazo[1,2-a]pyridin-3-yl)(3,4,5-trifluorophenyl)methanone (130 mg) obtained in step 4 was used instead of (8-bromo-2-cyclopropylimidazo[1,2-a]pyridin-3-yl)(3,4,5-trifluorophenyl)methanone in Example 95 (step 4), thereby obtaining (4-amino-3,5-difluorophenyl)(8-(1,2-dimethyl-6-(trifluoromethyl)-1H-benzo[d]imidazol-5-yl)-2-ethylimidazo[1,2-a]pyridin-3-yl)methanone (126 mg).

### Step 7, 8

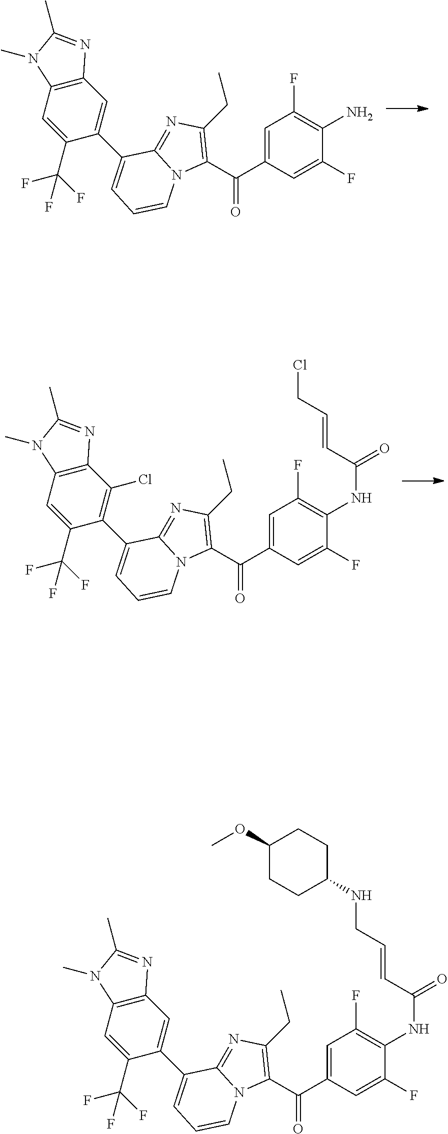

The procedures of Example 51 (steps 7, 8) were performed except that (4-amino-3,5-difluorophenyl)(8-(1,2-dimethyl-6-(trifluoromethyl)-1H-benzo[d]imidazol-5-yl)-2-ethylimidazo[1,2-a]pyridin-3-yl)methanone (126 mg) obtained in step 6 was used instead of (4-amino-3,5-difluorophenyl)(8-(1,2-dimethyl-6-(trifluoromethyl)-1H-benzo[d]imidazol-5-yl)-2-methylimidazo[1,2-a]pyridin-3-yl)methanone in Example 51 (step 7), thereby obtaining the title compound (69.7 mg).

### Example 100

### (E)-N-(4-(8-(4-chloro-1,2,6-trimethyl-1H-benzo[d]imidazol-5-yl)-1-ethylindolizine-3-carbonyl)-2,6-difluorophenyl)-4-(((1r,4r)-4-methoxycyclohexyl)amino)but-2-enamide

### Step 1

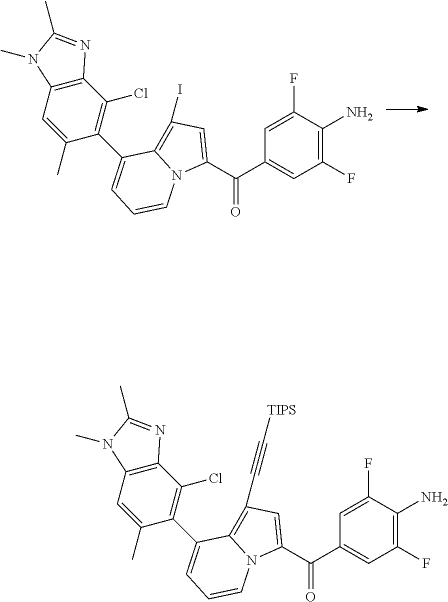

To a solution of (4-amino-3,5-difluorophenyl)(8-(4-chloro-1,2,6-trimethyl-1H-benzo[d]imidazol-5-yl)-1-iodoindolizin-3-yl)methanone (80.0 mg), bis(triphenylphosphine)palladium(II) dichloride (9.5 mg). Copper(I) iodide (2.6 mg) and triethyl amine (0.25 mL) in DMF (1.0 mL) was added ethynyl(triisopropyl)silane (0.91 mL) dropwise. After stirring at 60° C. for 3 hours, the reaction mixture was cooled to room temperature and diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-((triisopropylsilyl)ethynyl)indolizin-3-yl)methanone (60.9 mg).

### Step 2

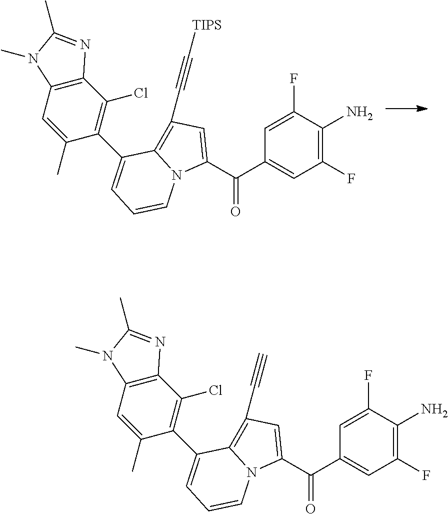

Tetrabutylammonium fluoride (a 1.0 M THF solution, 0.19 mL) was added to (4-amino-3,5-difluorophenyl)(8-(4-chloro-1,2,6-trimethyl-1H-benzo[d]imidazol-5-yl)-1-((triisopropylsilyl)ethynyl)indolizin-3-yl)methanone (60.0 mg) in THF (2.0 mL). After stirring at room temperature for 1 hour, the reaction mixture was diluted with EtOAc and water, extracted with EtOAc. The separated organic phase was washed with brine and dried over sodium sulfate, filtered, concentrated. The residue was purified by column chromatography (Hexane:ethyl acetate:methanol), thereby obtaining (4-amino-3,5-difluorophenyl)(8-(4-chloro-1,2,6-trimethyl-1H-benzo[d]imidazol-5-yl)-1-ethnylindolizin-3-yl)methanone (29.2 mg).

### Step 3

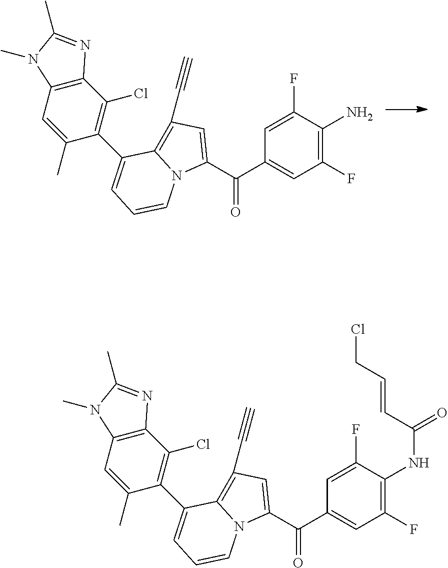 [SMILES: C#Cc1cc(C(=O)c2cc(F)c(N)c(F)c2)n2cccc(-c3c(C)cc4c(nc(C)n4C)c3Cl)c12 ; C#Cc1cc(C(=O)c2cc(F)c(NC(=O)/C=C/CCl)c(F)c2)n2cccc(-c3c(C)cc4c(nc(C)n4C)c3Cl)c12]

The procedure of Example 87 (step 2) was performed except that (4-amino-3,5-difluorophenyl)(8-(4-chloro-1,2,6-trimethyl-1H-benzo[d]imidazol-5-yl)-1-ethnylindolizin-3-yl)methanone (29.2 mg) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-ethnylindolizine-3-carbonyl)-2,6-difluorophenyl)but-2-enamide (30.3 mg) as a crude product.

### Step 4

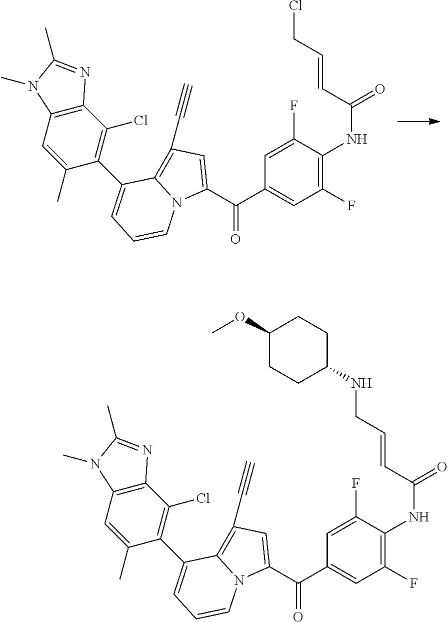 [SMILES: C#Cc1cc(C(=O)c2cc(F)c(NC(=O)/C=C/CCl)c(F)c2)n2cccc(-c3c(C)cc4c(nc(C)n4C)c3Cl)c12 ; C#Cc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12]

The procedure of Example 87 (step 3) was performed except that (E)-4-chloro-N-(4-(8-(4-chloro-1,2,6-trimethyl-1H-benzo[d]imidazol-5-yl)-1-ethnylindolizine-3-carbonyl)-2,6-difluorophenyl)but-2-enamide (30.3 mg) was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound (12.6 mg).

### Example 101

### (E)-N-(4-(8-(4-chloro-1,2,6-trimethyl-1H-benzo[d]imidazol-5-yl)-1-(1-methyl-1H-pyrazol-4-yl)indolizine-3-carbonyl)-2,6-difluorophenyl)-4-(((1r,4r)-4-methoxycyclohexyl)amino)but-2-enamide

The title compound (28.6 mg) was obtained using the same procedure as that of Example 88.

### Synthesis of (4-amino-3,5-difluorophenyl)(8-(5,5-dimethyl-1,3,2-dioxaborinan-2-yl)indolizin-3-yl)methanone

### Step 1

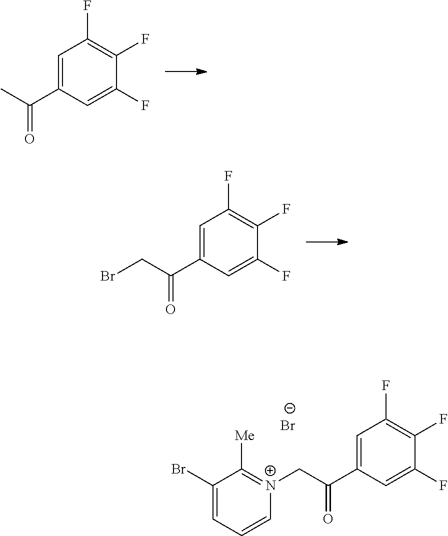 [SMILES: CC(=O)c1cc(F)c(F)c(F)c1 ; O=C(CBr)c1cc(F)c(F)c(F)c1 ; Cc1c(Br)ccc[n+]1CC(=O)c1cc(F)c(F)c(F)c1 ; [Br-]]

CuBr2 (4.23 g, 19.0 mmol) was added to a solution of 3′,4′,5′-trifluoroacetophenone (1.50 g. 8.61 mmol) in EtOAc (22.0 mL) and the reaction heated to 70° C. overnight. After cooling, the reaction was filtered through Celite and the filtrate washed sequentially with sat. aq. NaHCO₃ (3×) and brine, then dried (MgSO₄) and evaporated to give intermediate 2-bromo-1-(3,4,5-trifluorophenyl)ethan-1-one.

3-Bromo-2-methylpyridine (1.64 mL, 14.2 mmol) was added to a solution of 2-bromo-1-(3,4,5-trifluorophenyl)ethan-1-one (1.80 g, 7.11 mmol) in THF (10.0 mL) and the reaction heated to 80° C. for 4 hours Further 3-bromo-2-methylpyridine (0.820 mL. 7.11 mmol) was added and heating continued overnight. After cooling, heptane (15 mL) was added and the resultant precipitate collected via filtration, washing with further heptane, and dried in vacuo to give 3-Bromo-2-methyl-1-[2-oxo-2-(3,4,5-trifluorophenyl)ethyl]pyridin-1-ium bromide. MS: [M]+=344.

### Step 2

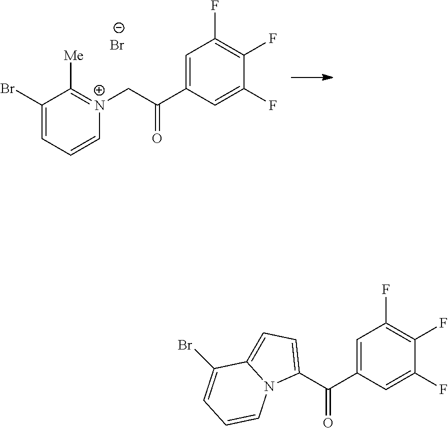 [SMILES: Cc1c(Br)ccc[n+]1CC(=O)c1cc(F)c(F)c(F)c1 ; [Br-] ; O=C(c1cc(F)c(F)c(F)c1)c1ccc2c(Br)cccn12]

A mixture of DMF (4.08 mL, 52.7 mmol) and dimethyl sulfate (4.99 mL, 52.7 mmol) was heated to 80° C. for 2 hours After cooling, this solution was added to a separate solution of 3-bromo-2-methyl-1-[2-oxo-2-(3,4,5-trifluorophenyl)ethyl]pyridin-1-ium bromide (2.80 g, 6.59 mmol) in DMF (10 mL) and the reaction stirred at RT for 30 min. N,N-Diisopropylethylamine (9.18 mL, 52.7 mmol) was added and the resulting suspension allowed to stand for 30 min. H₂O (˜50 mL) was added and, after standing for a further 30 min, the precipitate was collected via filtration, washing with further water, and the solid dried in vacuo. This solid was re-crystallised from boiling EtOAc (˜25 mL) to give 8-Bromo-3-(3,4,5-trifluorobenzoyl)indolizine. MS: [M+H]+=354.

### Step 3

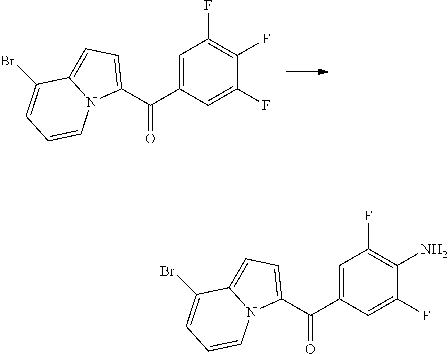 [SMILES: O=C(c1cc(F)c(F)c(F)c1)c1ccc2c(Br)cccn12 ; Nc1c(F)cc(C(=O)c2ccc3c(Br)cccn23)cc1F]

A 5 L pressure vessel was charged with 8-Bromo-3-(3,4,5-trifluorobenzoyl)indolizine (208 g, 0.59 mol). THF (2 L), and 28% NH₃ aq (830 ml), and heated at 135° C. with stirring for 24 hours. The mixture was cooled to rt, poured into ice-water (1 L), and stirred for 2 hours. The precipitate was collected by filtration and rinsed with water (1 L) to give a crude solid. The solid was added EtOAc (1 L) and heated at 65° C. To the mixture was slowly added hexane (2 L) then cooled down to rt. After stirring at rt for 20 hours, the solid was collected by filtration, rinsed with Hexane/EtOAc (3/1), dried in vacuo to give 4-(8-Bromoindolizine-3-carbonyl)-2,6-difluoroaniline. MS: [M+H]+=351.

### Step 4

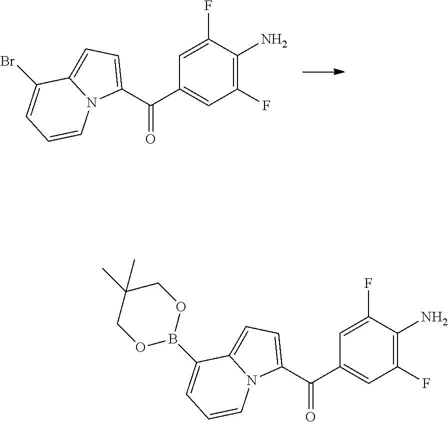 [SMILES: Nc1c(F)cc(C(=O)c2ccc3c(Br)cccn23)cc1F ; CC1(C)COB(c2cccn3c(C(=O)c4cc(F)c(N)c(F)c4)ccc23)OC1]

A 10 L 4-necked flask was charged with 4-(8-Bromoindolizine-3-carbonyl)-2,6-difluoroaniline (396 g. 1.13 mol), dry THF (3.8 L), and KOAc (188 g. 1.92 mol, 1.7 eq). The mixture was de-gassed (nitrogen gas was passed through the mixture at 50° C. for 40 min) and the mixture was added PdCl₂(dppf)-CH₂Cl₂ (28.6 g, 0.035 mol) and Bis(neopentyl Glycolato) diboron (331 g, 1.47 mol). The resulting mixture was stirred at 70° C. for 10 hours. After cooled down to rt, the mixture was poured into ice-water (4 L), added EtOAc (4 L), and filtered (celite). The filtrate was separated and the aq phase was re-extracted with EtOAc (2 L). Organic phases were combined and washed with water (3.5 L), sat NaCl aq (3.5 L), dried (Na₂SO₄), filtered, and concentrated. The resulting crude material was purified by column chromatography (SiO₂ 10 kg, CH₂Cl₂/EtOAc=10/1 to 3/1) and the resulting solid was further purified by washing with Hexane (500 ml) to give (4-amino-3,5-difluorophenyl)(8-(5,5-dimethyl-1,3,2-dioxaborinan-2-yl)indolizin-3-yl)methanone.

### Synthesis of 1,2-dimethyl-5-(4,4,5,5-tetramethyl-1,3,2-dioxaborolan-2-yl)-6-(trifluoromethyl)-1H-benzo[d]imidazole

### Step 1

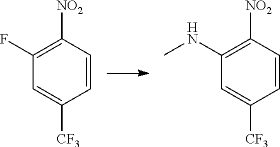

Methylamine (a 2M THF solution. 36 mL) was added to a solution of 2-fluoro-1-nitro-4-(trifluoromethyl)benzene (5.00 g) in THF (60 mL). After stirring at room temperature for 1 hour, the reaction mixture was concentrated under reduced pressure, thereby obtaining crude N-methyl-2-nitro-5-(trifluoromethyl)aniline.

### Step 2

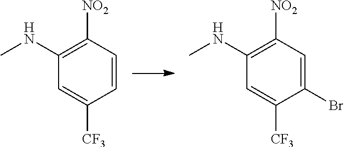 [SMILES: CNc1cc(C(F)(F)F)ccc1[N+](=O)[O-] ; CNc1cc(C(F)(F)F)c(Br)cc1[N+](=O)[O-]]

Crude N-methyl-2-nitro-5-(trifluoromethyl)aniline obtained in step 1 and N-bromosuccinimide (5.11 g) were added to acetic acid (80 ml). After the mixture was refluxed for 1 hour, the mixture was cooled to room temperature and poured into water. The resulting solid was collected by filtration, thereby obtaining crude 4-bromo-N-methyl-2-nitro-5-(trifluoromethyl)aniline (6.63 g).

### Step 3

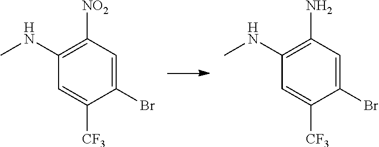 [SMILES: CNc1cc(C(F)(F)F)c(Br)cc1[N+](=O)[O-] ; CNc1cc(C(F)(F)F)c(Br)cc1N]

A suspension of crude 4-bromo-N-methyl-2-nitro-5-(trifluoromethyl)aniline (6.63 g) obtained in step 2 and iron powder (6.19 g) in a 2M ammonium chloride aqueous solution (55 mL). THE (110 mL), and methanol (110 mL) was stirred at 70° C. for 3 hours. The reaction mixture was concentrated under reduced pressure and the resulting mixture was diluted with water and ethyl acetate, followed by filtrating off the insoluble matter. The organic layer was separated and washed with a saturated sodium chloride solution, followed by drying over sodium sulfate. The solvent was evaporated under reduced pressure, and the obtained residue was purified by column chromatography (hexane:ethyl acetate), thereby obtaining 4-bromo-N1-methyl-5-(trifluoromethyl)benzene-1,2-diamine (5.25 g).

### Step 4

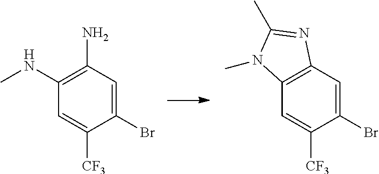 [SMILES: CNc1cc(C(F)(F)F)c(Br)cc1N ; Cc1nc2cc(Br)c(C(F)(F)F)cc2n1C]

Concentrated hydrochloric acid (610 μL) was added to a suspension of 4-bromo-N1-methyl-5-(trifluoromethyl)benzene-1,2-diamine (19.8 g) obtained in step 3 in 1,1,1-trimethoxyethane (130 mL), followed by stirring at room temperature for 1 hour. The reaction mixture was neutralized with a 5M sodium hydroxide aqueous solution. Water was added to the mixture, and extraction was performed with ethyl acetate, followed by washing the organic layer with a saturated sodium chloride solution. The washed layer was dried over sodium sulfate, and the solvent was evaporated under reduced pressure. The obtained residue was purified by column chromatography (hexane:ethyl acetate), thereby obtaining 5-bromo-1,2-dimethyl-6-(trifluoromethyl)-1H-benzo[d]imidazole (19.3 g).

### Step 5

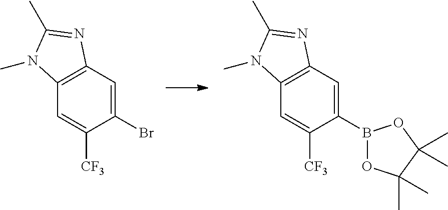

A suspension of 5-bromo-1,2-dimethyl-6-(trifluoromethyl)-1H-benzo[d]imidazole (12.6 g) obtained in step 4, potassium acetate (11.0 g), bis(pinacolato)diboron (17.5 g), (1,1′-bis(diphenylphosphino)ferrocene)palladium(II) dichloride dichloromethane adduct (3.51 g) in DMSO (190 mL) was stirred at 105° C. for 12 hours. Ethyl acetate and water were added to the reaction mixture, and the resulting mixture was filtered through celite. The aqueous layer was extracted with ethyl acetate, and the organic layer was washed with water and a saturated sodium chloride solution. The washed organic layer was dried over sodium sulfate, and the solvent was evaporated under reduced pressure, followed by purifying the obtained residue by column chromatography (hexane:ethyl acetate), thereby obtaining 1,2-dimethyl-5-(4,4,5,5-tetramethyl-1,3,2-dioxaborolan-2-yl)-6-(trifluoromethyl)-1H-benzo[d]imidazole (6.61 g).

### Synthesis of 2-ethyl-1-methyl-5-(4,4,5,5-tetramethyl-1,3,2-dioxaborolan-2-yl)-6-(trifluoromethyl)-1H-benzo[d]imidazole

### Step 1

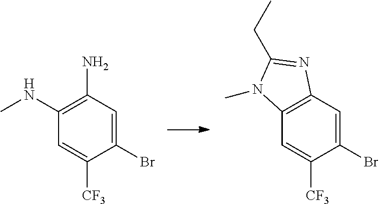 [SMILES: CNc1cc(C(F)(F)F)c(Br)cc1N ; CCc1nc2cc(Br)c(C(F)(F)F)cc2n1C]

Concentrated hydrochloric acid (300 μL) was added to a suspension of 4-bromo-N1-methyl-5-(trifluoromethyl)benzene-1,2-diamine (5.25 g) in 1,1,1-triethoxypropane (30 mL), followed by stirring at room temperature overnight. The solvent was evaporated under reduced pressure, and the obtained residue was purified by column chromatography (hexane:ethyl acetate), thereby obtaining 5-bromo-2-ethyl-1-methyl-6-(trifluoromethyl)-1H-benzo[d]imidazole (5.22 g).

### Step 2

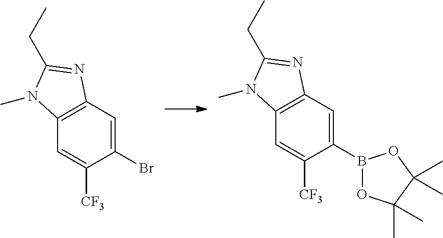

A suspension of 5-bromo-2-ethyl-1-methyl-6-(trifluoromethyl)-1H-benzo[d]imidazole (1.00 g) obtained in step 1, potassium acetate (959 mg), bis(pinacolato)diboron (1.24 g). (1,1′-bis(diphenylphosphino)ferrocene)palladium(II) dichloride dichloromethane adduct (266 mg) in DMSO (15 mL) was stirred at 100° C. for 6 hours. Ethyl acetate and water were added to the reaction mixture to separate the organic layer, and the organic layer was washed with a saturated sodium chloride solution. The washed organic layer was dried over sodium sulfate, and the solvent was evaporated under reduced pressure, followed by purifying the obtained residue by column chromatography (hexane:ethyl acetate), thereby obtaining 2-ethyl-1-methyl-5-(4,4,5,5-tetramethyl-1,3,2-dioxaborolan-2-yl)-6-(trifluoromethyl)-1H-benzo[d]imidazole (942 mg).

### Synthesis of 5-iodo-4-methoxy-1,2,6-trimethyl-1H-benzo[d]imidazole

### Step 1

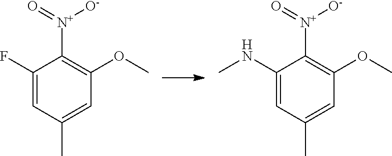

Methylamine (2M THF solution. 38.2 mL, 76.4 mmol) was added to a 1-fluoro-3-methoxy-5-methyl-2-nitrobenzene (4.04 g, 21.8 mmol). After the reaction mixture was heated at 70° C. by microwave irradiation for 12 hours, the mixture was cooled to room temperature. The mixture was diluted with EtOAc, washed with H₂O and brine then dried over Na₂SO₄ and evaporated, thereby obtaining crude 3-methoxy-N,5-dimethyl-2-nitroaniline (4.29 g, 100%).

### Step 2

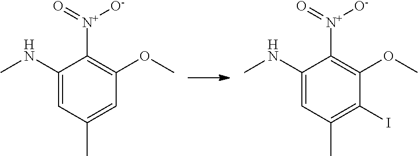

N-Iodosuccinimide (6.72 g, 29.9 mmol) was added to a solution of 3-methoxy-N,5-dimethyl-2-nitroaniline (5.33 g, 27.2 mmol) in DMF (53.3 mL). After stirring at 45° C. for 2 hours, the mixture was cooled to room temperature and H₂O and saturated aqueous NaHCO₃ was added to the mixture. The resulting solid was collected by filtration, and vacuum-dried at 40° C., thereby obtaining crude 4-iodo-3-methoxy-N,5-dimethyl-2-nitroaniline (8.48 g, 97%).

### Step 3

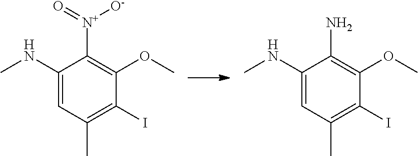

NH₄Cl (3.52 g, 65.8 mmol) and iron (7.35 g, 132 mmol) were added to a solution of 4-iodo-3-methoxy-N,5-dimethyl-2-nitroaniline (8.48 g, 26.3 mmol) in MeOH (43.8 mL). THF (43.8 mL) and H₂O (21.9 mL). After stirring at 60° C. for 3 hours, the mixture was cooled to room temperature and diluted with EtOAc then filtered through a Celite pad.

The filtrate was washed with H₂O and brine then dried over Na₂SO₄ and evaporated. The residue was purified by column chromatography on silica gel (hexane/EtOAc), thereby obtaining 4-iodo-3-methoxy-N1,5-dimethylbenzene-1,2-diamine (6.68 g, 87%).

### Step 4

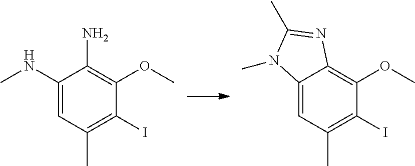

conc. HCl (95.4 μL, 1.15 mmol) and AcOH (656 μL, 11.5 mmol) were added to a solution of 4-iodo-3-methoxy-N1,5-dimethylbenzene-1,2-diamine

(3.35 g, 11.5 mmol) in trimethyl orthoacetate (20.4 mL, 160 mmol). After stirring at room temperature for 14 hours, the reaction was quenched with 10 mol/L aqueous NaOH (1.33 mL, 12.7 mmol). The reaction mixture was diluted with EtOAc, washed with H₂O and brine then dried over Na₂SO₄ and evaporated. The residue was purified by column chromatography on silica gel (hexane/EtOAc), thereby obtaining 5-iodo-4-methoxy-1,2,6-trimethyl-1H-benzo[d]imidazole (2.09 g, 58%).

### Synthesis of (7-bromo-1H-indol-3-yl)(3,4,5-trifluorophenyl)methanone

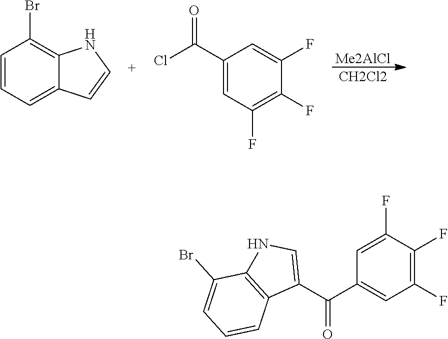

Dimethylaluminum chloride solution (1.0 M in hexanes, 49 mL, 49 mmol) was added to an ice bath cooled solution of 7-bromo-1H-indole (4.80 g. 24.5 mmol) in DCM (100 mL) and stirred at ambient temperature for 30 min. 3,4,5-trifluorobenzoyl chloride (4.81 mL, 36.7 mmol) was added thereto, and the mixture was stirred at 0° C. for 20 min. 2N Hydrochloric acid was slowly added thereto and the mixture was diluted with the mixture of chloroform (80 mL) and ethanol (20 mL). The organic layer was separated and washed with aqueous NaHCO₃ solution and brine, and then dried (Na₂SO₄) and evaporated. The residue was diluted with IPE and hexane, and the precipitate was collected via filtration to give the title compound (4.54 g). The remained filtrate was evaporated and purified by column chromatography on silica gel (gradient elution. 10-50%, EtOAc-hexane) to give (7-bromo-1H-indol-3-yl)(3,4,5-trifluorophenyl)methanone (2.52 g). MS: [M+H]⁺=354.356.

### Example 102-1 and 102-2

### (E)-N-(4-(2-ethyl-8-(4-methoxy-1,2-dimethyl-6-(trifluoromethyl)-1H-benzo[d]imidazol-5-yl)imidazo[1,2-a]pyridine-3-carbonyl)-2,6-difluorophenyl)-4-(((1r,4r)-4-methoxycyclohexyl)amino)but-2-enamide

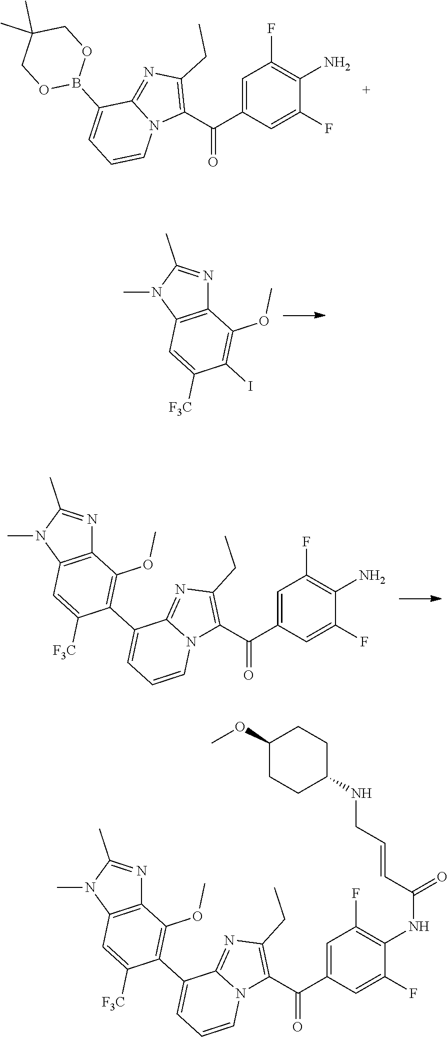

The procedure of Example 102 was performed in a similar manner as Examples 83 and 99 to give the racemic title compound. The racemate (40.0 mg) was dissolved in EtOH and purified by preparative HPLC (CHIRALPAK IC, hexane-EtOH (0.1% Et N)) to afford (E)-N-(4-(2-ethyl-8-(4-methoxy-1,2-dimethyl-6-(trifluoromethyl)-1H-benzo[d]imidazol-5-yl)imidazo[1,2-a]pyridine-3-carbonyl)-2,6-difluorophenyl)-4-(((1r,4r)-4-methoxycyclohexyl)amino)but-2-enamide (Example 102-1, single isomer. 1st-eluting isomer) and (E)-N-(4-(2-ethyl-8-(4-methoxy-1,2-dimethyl-6-(trifluoromethyl)-1H-benzo[d]imidazol-5-yl)imidazo[1,2-a]pyridine-3-carbonyl)-2,6-difluorophenyl)-4-(((1r,4r)-4-methoxycyclohexyl)amino)but-2-enamide (Example 102-2, single isomer. 2nd-eluting isomer).

### Example 103

### (E)-N-(4-(8-(4-chloro-1,2,6-trimethyl-1H-benzo[d]imidazol-5-yl)-1-(methoxymethyl)indolizine-3-carbonyl)-2,6-difluorophenyl)-4-(((1r,4r)-4-methoxycyclohexyl)amino)but-2-enamide

### Step 1

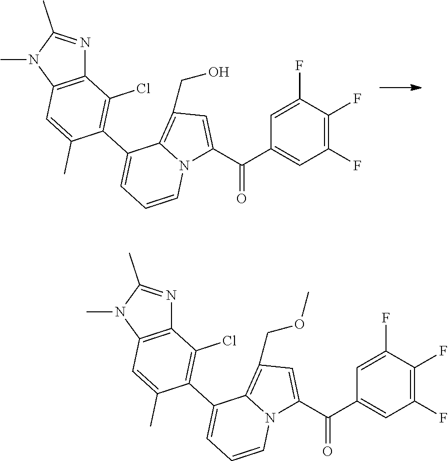 [SMILES: Cc1cc2c(nc(C)n2C)c(Cl)c1-c1cccn2c(C(=O)c3cc(F)c(F)c(F)c3)cc(CO)c12 ; COCc1cc(C(=O)c2cc(F)c(F)c(F)c2)n2cccc(-c3c(C)cc4c(nc(C)n4C)c3Cl)c12]

To a mixture of (8-(4-chloro-1,2,6-trimethyl-1H-benzo[d]imidazol-5-yl)-1-(hydroxymethyl)indolizin-3-yl)(3,4,5-trifluorophenyl)methanone (53.0 mg, 0.106 mmol) and methyl iodide (0.265 mL, 0.426 mmol) in DMF (1.0 mL) was added sodium hydride (60 wt %, 8.52 mg, 0.213 mmol). After stirring the mixture at room temperature for 1 hour, methyl iodide (0.265 mL, 0.426 mmol) and sodium hydride (60 wt %, 8.52 mg, 0.213 mmol) was added thereto. The mixture was stirred at room temperature overnight, and diluted with EtOAc and sat. NaHCO₃ aq. The separated organic layer was washed with brine, dried over Na₂SO₄, and evaporated. The residue was purified by column chromatography on silica gel (hexane-EtOAc-MeOH) to obtain (8-(4-chloro-1,2,6-trimethyl-1H-benzo[d]imidazol-5-yl)-1-(methoxymethyl)indolizin-3-yl)(3,4,5-trifluorophenyl)methanone.

### Step 2

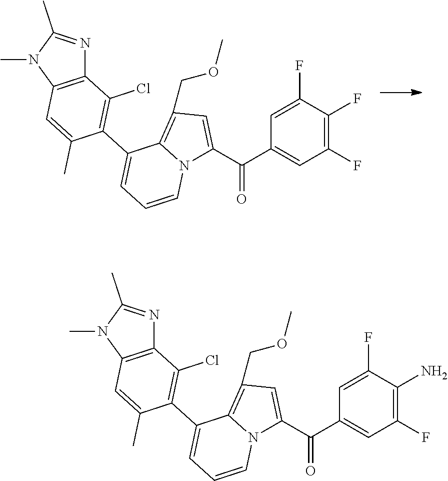 [SMILES: COCc1cc(C(=O)c2cc(F)c(F)c(F)c2)n2cccc(-c3c(C)cc4c(nc(C)n4C)c3Cl)c12 ; COCc1cc(C(=O)c2cc(F)c(N)c(F)c2)n2cccc(-c3c(C)cc4c(nc(C)n4C)c3Cl)c12]

To a solution of (8-(4-chloro-1,2,6-trimethyl-1H-benzo[d]imidazol-5-yl)-1-(methoxymethyl)indolizin-3-yl)(3,4,5-trifluorophenyl)methanone (35.4 mg, 0.0692 mmol) in 1,4-dioxane (2.0 mL) was added 28-30% NH₃ aq. (3.0 mL). The reaction mixture was heated at 130° C. under microwave irradiation for 12 hours. After cooling, the mixture was diluted with EtOAc, washed with brine then dried over Na₂SO₄ and evaporated. The residue was purified by column chromatography on silica gel (hexane-EtOAc/EtOH(4/1)) to obtain (4-amino-3,5-difluorophenyl)(8-(4-chloro-1,2,6-trimethyl-1H-benzo[d]imidazol-5-yl)-1-(methoxymethyl)indolizin-3-yl)methanone.

### Step 3

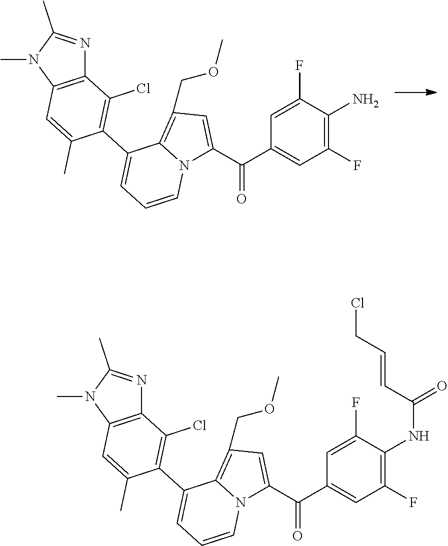 [SMILES: COCc1cc(C(=O)c2cc(F)c(N)c(F)c2)n2cccc(-c3c(C)cc4c(nc(C)n4C)c3Cl)c12 ; COCc1cc(C(=O)c2cc(F)c(NC(=O)/C=C/CCl)c(F)c2)n2cccc(-c3c(C)cc4c(nc(C)n4C)c3Cl)c12]

The procedure of Example 87 (step 2) was performed except that (4-amino-3,5-difluorophenyl)(8-(4-chloro-1,2,6-trimethyl-1H-benzo[d]imidazol-5-yl)-1-(methoxymethyl)indolizin-3-yl)methanone (23.1 mg, 0.0454 mmol) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-(methoxymethyl)indolizine-3-carbonyl)-2,6-difluorophenyl)but-2-enamide.

### Step 4

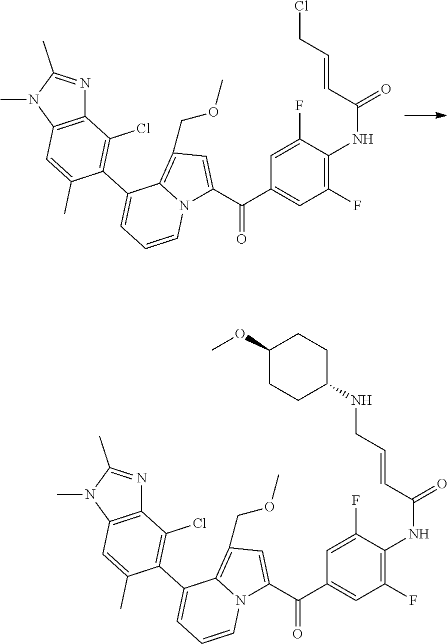 [SMILES: COCc1cc(C(=O)c2cc(F)c(NC(=O)/C=C/CCl)c(F)c2)n2cccc(-c3c(C)cc4c(nc(C)n4C)c3Cl)c12 ; COCc1cc(C(=O)c2cc(F)c(NC(=O)/C=C/CN[C@H]3CC[C@H](OC)CC3)c(F)c2)n2cccc(-c3c(C)cc4c(nc(C)n4C)c3Cl)c12]

The procedure of Example 87 (step 3) was performed except that (E)-4-chloro-N-(4-(8-(4-chloro-1,2,6-trimethyl-1H-benzo[d]imidazol-5-yl)-1-(methoxymethyl)indolizine-3-carbonyl)-2,6-difluorophenyl)but-2-enamide (8.80 mg, 0.0144 mmol) was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound.

### Example 104

### (E)-N-(4-(8(4-chloro-1,2-dimethyl-6-(trifluoromethyl)-1H-benzo[d]imidazol-5-yl)indolizine-3-carbonyl)-2,3,6-trifluorophenyl)-4-(((1r,4r)-4-methoxycyclohexyl)amino)but-2-enamide

The procedure was performed in the same manner as Example 32 and synthesis of (4-amino-3,5-difluorophenyl)(8-(5,5-dimethyl-1,3,2-dioxaborinan-2-yl)indolizin-3-yl)methanone, except that 1-(2,3,4,5-tetrafluorophenyl)ethan-1-one was used instead of 3′,4′,5′-trifluoroacetophenone to give the title compound.

### Example 105-1 and 105-2

### (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-((1-methylcyclopropyl)amino)but-2-enamide

The procedure of Example 105 was performed in the same manner as Example 83 to give the racemic title compound. The racemate (30.0 mg) was dissolved in EtOH and purified by preparative HPLC (CHIRALPAK IC, hexane-EtOH (0.1% Eₜ₃N)) to afford (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-((1-methylcyclopropyl)amino)but-2-enamide (Example 105-1, single isomer. 1st-eluting isomer) and (E)-N-(2,6-difluoro-4-(8-(4-methoxy-1,2-dimethyl-6-(trifluoromethyl)-1H-benzo[d]imidazol-5-yl)imidazo[1,2-a]pyridine-3-carbonyl)phenyl)-4-((1-methylcyclopropyl)amino)but-2-enamide (Example 105-2, single isomer. 2nd-eluting isomer).

### Example 106

### 8-(4-chloro-1,2,6-trimethyl-1H-benzo[d]imidazol-5-yl)-3-(3,5-difluoro-4-((E)-4-(((1r,4r)-4-methoxycyclohexyl)amino)but-2-enamido)benzol)-N,N-dimethylindolizine-1-carboxamide

### Step 1-2

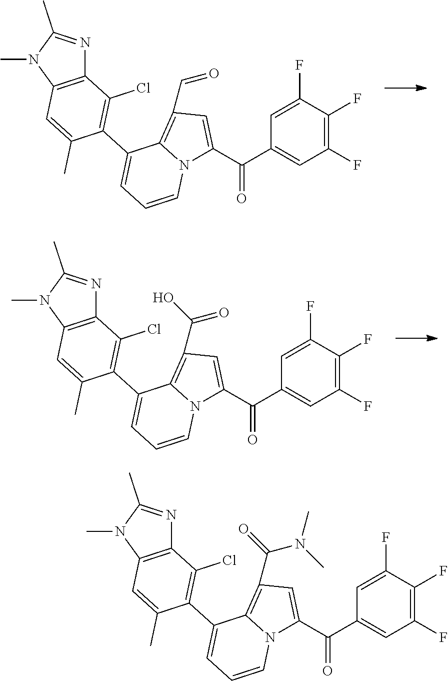

To a mixture of 8-(4-chloro-1,2,6-trimethyl-1H-benzo[d]imidazol-5-yl)-3-(3,4,5-trifluorobenzoyl)indolizine-1-carbaldehyde (50.0 mg, 0.101 mmol), NaPH₂O₄ aq. (0.70M. 1.01 mL, 0.706 mmol), 2-methyl-2-butene (0.534 mL, 5.04 mmol), sulfamic acid (1% mg, 2.02 mmol) in tert-butanol (0.50 mL) and THF (0.50 mL) was added sodium chlorite (219 mg, 0.471 mmol), and stirred at room temperature for 15 minutes. EtOAc and sat. NaHCO₃ aq. were added thereto, and the separated organic layer was washed with brine, dried over Na₂SO₄, and evaporated to obtain 8-(4-chloro-1,2,6-trimethyl-1H-benzo[d]imidazol-5-yl)-3-(3,4,5-trifluorobenzoyl)indolizine-1-carboxylic acid. The resulting crude product was diluted with DMF (1.0 mL), then dimethylamine (2.0M, 0.0757 mL, 0.151 mmol), 1-(3-dimethylaminopropyl)-3-ethylcarbodiimide hydrochloride (29.0 mg, 0.151 mmol), 1-hydroxybenzotriazole hydrate (23.2 mg, 0.151 mmol) and triethylamine (0.211 mL, 0.151 mmol) were added thereto. After stirring at room temperature for 1 hour and 50° C. for 30 minutes, the mixture was diluted with EtOAc and sat. NaHCO₃ aq., and the separated organic layer was washed with brine, dried over Na₂SO₄, and evaporated. The residue was purified by column chromatography on silica gel (hexane-EtOAc-MeOH) to obtain 8-(4-chloro-1,2,6-trimethyl-1H-benzo[d]imidazol-5-yl)-N,N-dimethyl-3-(3,4,5-trifluorobenzoyl)indolizine-1-carboxamide.

### Step 3

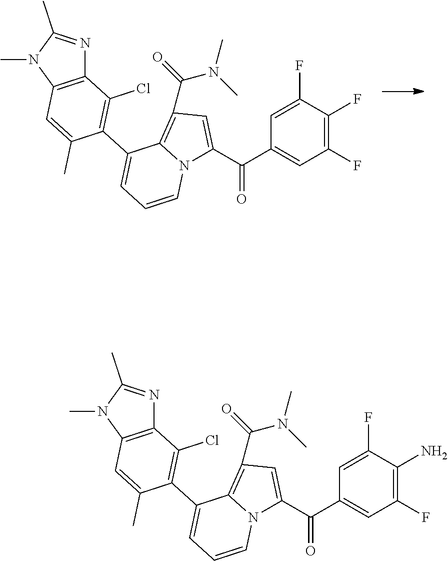 [SMILES: Cc1cc2c(nc(C)n2C)c(Cl)c1-c1cccn2c(C(=O)c3cc(F)c(F)c(F)c3)cc(C(=O)N(C)C)c12 ; Cc1cc2c(nc(C)n2C)c(Cl)c1-c1cccn2c(C(=O)c3cc(F)c(N)c(F)c3)cc(C(=O)N(C)C)c12]

The procedure of Example 103 (step 2) was performed except that 8-(4-chloro-1,2,6-trimethyl-1H-benzo[d]imidazol-5-yl)-N,N-dimethyl-3-(3,4,5-trifluorobenzoyl)indolizine-1-carboxamide (33.2 mg, 0.0616 mmol) was used instead of (8-(4-chloro-1,2,6-trimethyl-1H-benzo[d]imidazol-5-yl)-1-(methoxymethyl)indolizin-3-yl)(3,4,5-trifluorophenyl)methanone, thereby obtaining 3-(4-amino-3,5-difluorobenzoyl)-8-(4-chloro-1,2,6-trimethyl-1H-benzo[d]imidazol-5-yl)-N,N-dimethylindolizine-1-carboxamide.

### Step 4-5

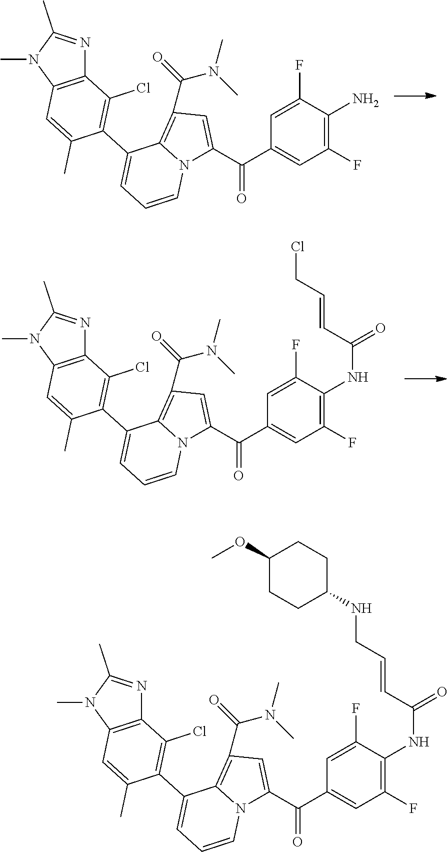 [SMILES: Cc1cc2c(nc(C)n2C)c(Cl)c1-c1cccn2c(C(=O)c3cc(F)c(N)c(F)c3)cc(C(=O)N(C)C)c12 ; Cc1cc2c(nc(C)n2C)c(Cl)c1-c1cccn2c(C(=O)c3cc(F)c(NC(=O)/C=C/CCl)c(F)c3)cc(C(=O)N(C)C)c12 ; CO[C@H]1CC[C@H](NC/C=C/C(=O)Nc2c(F)cc(C(=O)c3cc(C(=O)N(C)C)c4c(-c5c(C)cc6c(nc(C)n6C)c5Cl)cccn34)cc2F)CC1]

The procedure of Example 87 (step 2) was performed except that 3-(4-amino-3,5-difluorobenzoyl)-8-(4-chloro-1,2,6-trimethyl-1H-benzo[d]imidazol-5-yl)-N,N-dimethylindolizine-1-carboxamide (27.1 mg, 0.0506 mmol) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-8-(4-chloro-1,2,6-trimethyl-1H-benzo[d]imidazol-5-yl)-3-(4-(4-chlorobut-2-enamido)-3,5-difluorobenzoyl)-N,N-dimethylindolizine-1-carboxamide. Then the procedure of Example 87 (step 3) was performed except that (E)-8-(4-chloro-1,2,6-trimethyl-1H-benzo[d]imidazol-5-yl)-3-(4-(4-chlorobut-2-enamido)-3,5-difluorobenzoyl)-N,N-dimethylindolizine-1-carboxamide was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound.

### Example 107

### 8-(4-chloro-1,2,6-trimethyl-1H-benzo[d]imidazol-5-yl)-3-(3,5-difluoro-4-((E)-4-(((1r,4r)-4-methoxycyclohexyl)amino)but-2-enamido)benzoyl)indolizine-1-carboxamide

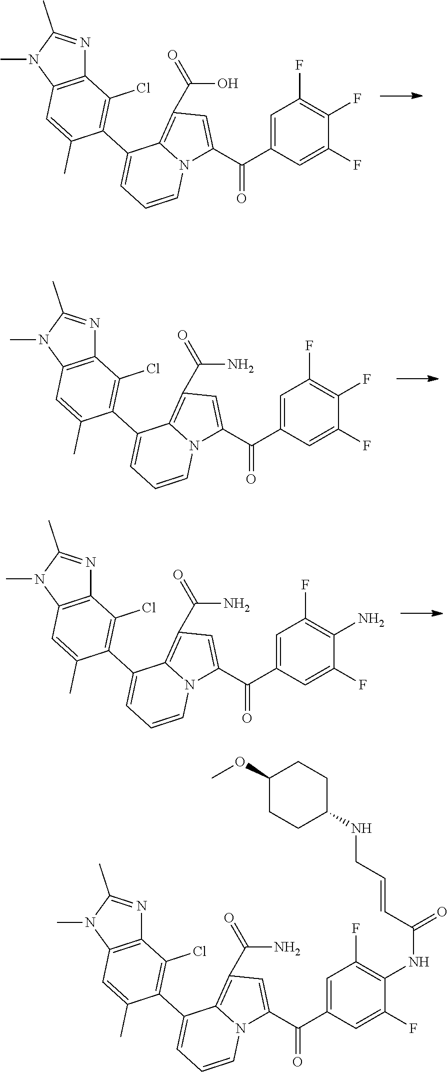

The procedure of Example 107 was performed in the same manner as Example 106 to give the title compound.

### Example 108

### (E)-N-(4-(8-(4-chloro-1,2,6-trimethyl-1H-benzo[d]imidazol-5-yl)-1-(cyanomethyl)indolizine-3-carbonyl)-2,6-difluorophenyl)-4-(((1r,4r)-4-methoxycyclohexyl)amino)but-2-enamide

### Step 1

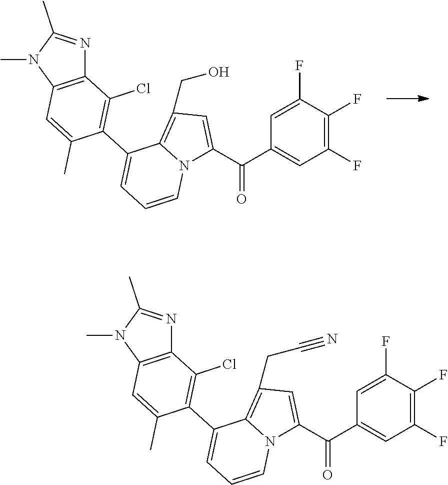 [SMILES: Cc1cc2c(nc(C)n2C)c(Cl)c1-c1cccn2c(C(=O)c3cc(F)c(F)c(F)c3)cc(CO)c12 ; Cc1cc2c(nc(C)n2C)c(Cl)c1-c1cccn2c(C(=O)c3cc(F)c(F)c(F)c3)cc(CC#N)c12]

(8-(4-chloro-1,2,6-trimethyl-1H-benzo[d]imidazol-5-yl)-1-(hydroxymethyl)indolizin-3-yl)(3,4,5-trifluorophenyl)methanone (30 mg, 0.0603 mmol) in dichloromethane (2.0 mL) was slowly added to the mixture of trimethylsilylformonitrile (0.0321 mL, 0.241 mmol) in dichloromethane (35.2 mL) at 0° C. After stirring at the same temperature for 1 hour. EtOAc and sat. NaHCO₃ aq. were added thereto. The organic layer was separated and washed with brine, followed by drying over Na₂SO₄ and evaporation. The residue was purified by column chromatography (hexane:EtOAc/EtOH(4/1)) to obtain 2-(8-(4-chloro-1,2,6-trimethyl-1H-benzo[d]imidazol-5-yl)-3-(3,4,5-trifluorobenzoyl)indolizin-1-yl)acetonitrile.

### Step 2

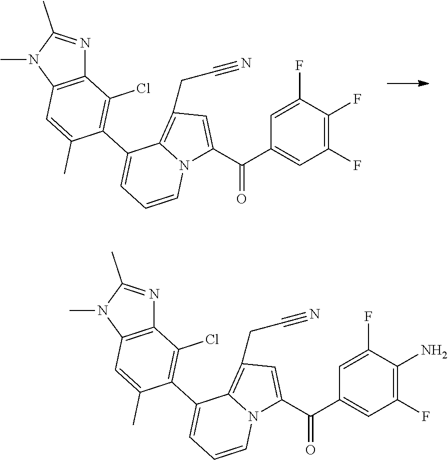 [SMILES: Cc1cc2c(nc(C)n2C)c(Cl)c1-c1cccn2c(C(=O)c3cc(F)c(F)c(F)c3)cc(CC#N)c12 ; Cc1cc2c(nc(C)n2C)c(Cl)c1-c1cccn2c(C(=O)c3cc(F)c(N)c(F)c3)cc(CC#N)c12]

The procedure of Example 103 (step 2) was performed except that 2-(8-(4-chloro-1,2,6-trimethyl-1H-benzo[d]imidazol-5-yl)-3-(3,4,5-trifluorobenzoyl)indolizin-1-yl)acetonitrile (24.8 mg, 0.0489 mmol) was used instead of (8-(4-chloro-1,2,6-trimethyl-1H-benzo[d]imidazol-5-yl)-1-(methoxymethyl)indolizin-3-yl)(3,4,5-trifluorophenyl)methanone, thereby obtaining 2-(3-(4-amino-3,5-difluorobenzoyl)-8-(4-chloro-1,2,6-trimethyl-1H-benzo[d]imidazol-5-yl)indolizin-1-yl)acetonitrile.

### Step 3-4

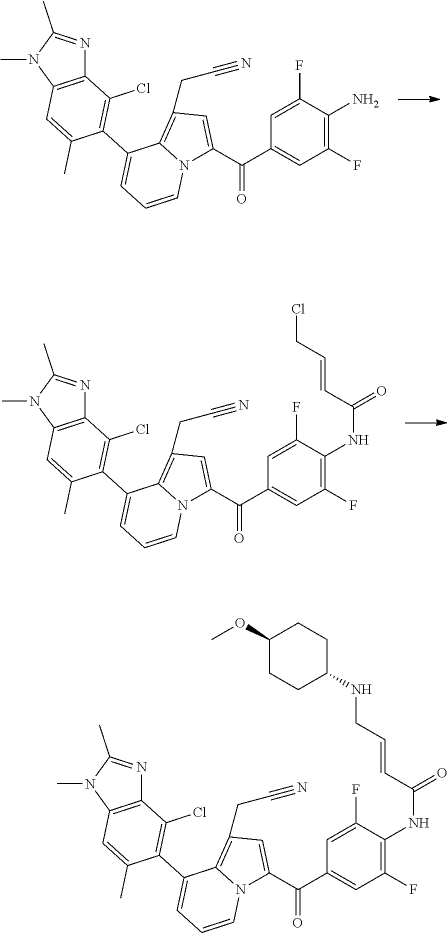 [SMILES: Cc1cc2c(nc(C)n2C)c(Cl)c1-c1cccn2c(C(=O)c3cc(F)c(N)c(F)c3)cc(CC#N)c12 ; Cc1cc2c(nc(C)n2C)c(Cl)c1-c1cccn2c(C(=O)c3cc(F)c(NC(=O)/C=C/CCl)c(F)c3)cc(CC#N)c12 ; CO[C@H]1CC[C@H](NC/C=C/C(=O)Nc2c(F)cc(C(=O)c3cc(CC#N)c4c(-c5c(C)cc6c(nc(C)n6C)c5Cl)cccn34)cc2F)CC1]

The procedure of Example 87 (step 2) was performed except that 2-(3-(4-amino-3,5-difluorobenzoyl)-8-(4-chloro-1,2,6-trimethyl-1H-benzo[d]imidazol-5-yl)indolizin-1-yl)acetonitrile (22.0 mg, 0.0437 mmol) was used instead of (4-amino-3,5-difluorophenyl)(8-(4-chloro-1,2,6-trimethyl-1H-benzo[d]imidazol-5-yl)-1-methylindolizin-3-yl)methanone, thereby obtaining (E)-4-chloro-N-(4-(8-(4-chloro-1,2,6-trimethyl-1H-benzo[d]imidazol-5-yl)-1-(cyanomethyl)indolizine-3-carbonyl)-2,6-difluorophenyl)but-2-enamide. Then the procedure of Example 87 (step 3) was performed except that (E)-4-chloro-N-(4-(8-(4-chloro-1,2,6-trimethyl-1H-benzo[d]imidazol-5-yl)-1-(cyanomethyl)indolizine-3-carbonyl)-2,6-difluorophenyl)but-2-enamide was used instead of (E)-4-chloro-N-(4-(8-(4-chloro-1,2,6-trimethyl-1H-benzo[d]imidazol-5-yl)-1-methylindolizine-3-carbonyl)-2,6-difluorophenyl)but-2-enamide, thereby obtaining the title compound.

### Example 109-1 and 109-2

### (E)-N-(2,6-difluoro-4(8-(4-methoxy-1,2-dimethyl-6-(trifluoromethyl)-1H-benzo[d]imidazol-5-yl)-2-methylimidazo[1,2-a]pyridine-3-carbonyl)phenyl)-4-((1-methylcyclopropyl)amino)but-2-enamide

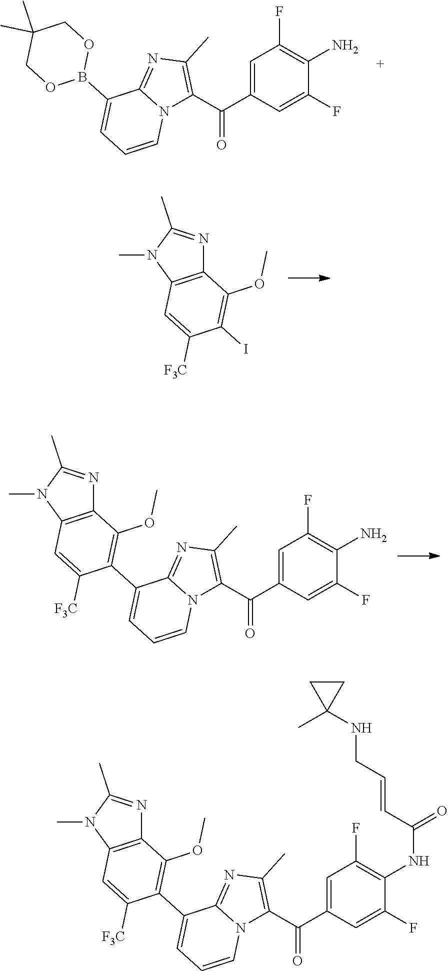

The procedure of Example 109 was performed in a similar manner as Examples 51 and 83 to give the racemic title compound. The racemate (30.6 mg) was dissolved in EtOH and purified by preparative HPLC (CHIRALPAK IC, hexane-EtOH (0.1% Et₃N)) to afford (E)-N-(2,6-difluoro-4-(8-(4-methoxy-1,2-dimethyl-6-(trifluoromethyl)-1H-benzo[d]imidazol-5-yl)-2-methylimidazo[1,2-a]pyridine-3-carbonyl)phenyl)-4-((1-methylcyclopropyl)amino)but-2-enamide (Example 109-1, single isomer, 1st-eluting isomer) and (E)-N-(2,6-difluoro-4-(8-(4-methoxy-1,2-dimethyl-6-(trifluoromethyl)-1H-benzo[d]imidazol-5-yl)-2-methylimidazo[1,2-a]pyridine-3-carbonyl)phenyl)-4-((1-methylcyclopropyl)amino)but-2-enamide (Example 109-2, single isomer. 2nd-eluting isomer).

### Example 110

### (E)-N-(2,6-difluoro-4-(8-(1-methyl-2-oxo-6-(trifluoromethyl)-2,3-dihydro-1H-benzo[d]imidazol-5-yl)indolizine-3-carbonyl)phenyl)-4-(((1r,4r)-4-methoxycyclohexyl)amino)but-2-enamide

### Step 1

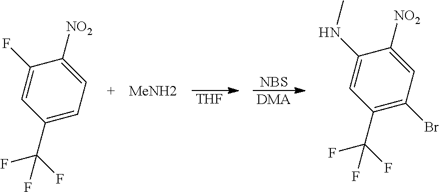

Methylamine (2M in THF solution, 30.1 mL, 60.2 mmol) was slowly added to a solution of 4-fluoro-3-nitrobenzotrifluoride (4.42 g, 20.1 mmol) in THF (4.2 mL) at 0° C. After stirring at room temperature for 30 minutes, water and EtOAc were added to the resulting mixture. The organic layer was separated, and washed with brine, followed by drying over sodium sulfate. After evaporation, to the obtained residue was added DMA (28.3 mL), and NBS (3.93 g, 22.1 mmol) was added thereto at room temperature. After stirring overnight, additional NBS (200 mg) was added thereto. After stirring at room temperature for 1.5 hours, water was added thereto, and the precipitates were collected by filtration and dried in vacuo at 50° C. to obtain 4-bromo-N-methyl-2-nitro-5-(trifluoromethyl)aniline.

### Step 2

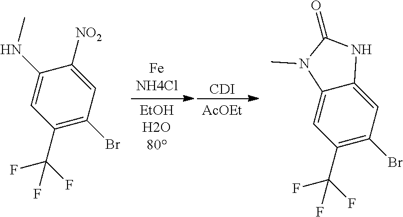 [SMILES: "AcOEt" ; CNc1cc(C(F)(F)F)c(Br)cc1[N+](=O)[O-] ; "CDI" ; Cn1c(=O)[nH]c2cc(Br)c(C(F)(F)F)cc21]

The mixture of 4-bromo-N-methyl-2-nitro-5-(trifluoromethyl)aniline (1.00 g, 3.34 mmol), iron powder (934 mg, 16.7 mmol) and ammonium chloride (894 mg, 16.7 mmol) in ethanol (20.1 mL) and water (4.0 mL) was stirred at 80° C. for 50 minutes. The resulting mixture was diluted with EtOAc. and the insoluble matters were removed by filtration. The filtrate was washed with saturated NaHCO₃ aq. and brine, dried over Na₂SO₄, and the solvent was removed by evaporation. To the residue was added EtOAc and CDI (2.0 g), and the mixture was stirred at 50° C. for 2 hours. The solvent of the mixture was evaporated, and the residue was diluted with water and 2N HCl aq. The precipitates were collected by filtration and dried in vacuo at 50° C. to obtain 5-bromo-1-methyl-6-(trifluoromethyl)-1,3-dihydro-2H-benzo[d]imidazol-2-one.

### Step 3

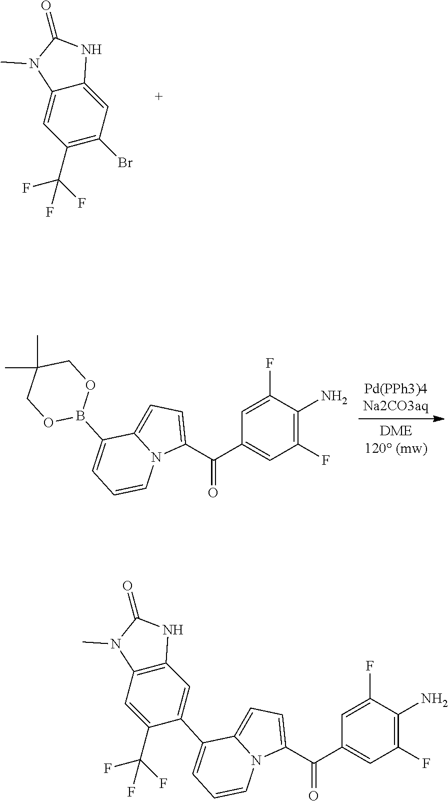 [SMILES: Cn1c(=O)[nH]c2cc(Br)c(C(F)(F)F)cc21 ; CC1(C)COB(c2cccn3c(C(=O)c4cc(F)c(N)c(F)c4)ccc23)OC1 ; Cn1c(=O)[nH]c2cc(-c3cccn4c(C(=O)c5cc(F)c(N)c(F)c5)ccc34)c(C(F)(F)F)cc21]

To a mixture of (4-amino-3,5-difluorophenyl)(8-(5,5-dimethyl-1,3,2-dioxaborinan-2-yl)indolizin-3-yl)methanone (110 mg), 5-bromo-1-methyl-6-(trifluoromethyl)-1,3-dihydro-2H-benzo[d]imidazol-2-one (59 mg) and Pd(PPh₃)₄ (18.5 mg) in 1,2-dimethoxyethane (2.0 mL) was added 2M aqueous sodium carbonate solution (0.180 mL). After reacting by microwave apparatus at 120° C. for 2 hours, the mixture was diluted with EtOAc and water. The organic layer was washed with brine, dried over sodium sulfate and evaporated under reduced pressure. The residue was purified by column chromatography on silica gel (CHCl₃-ethanol) to give crude 5-(3-(4-amino-3,5-difluorobenzoyl)indolizin-8-yl)-1-methyl-6-(trifluoromethyl)-1,3-dihydro-2H-benzo[d]imidazol-2-one.

### Step 4

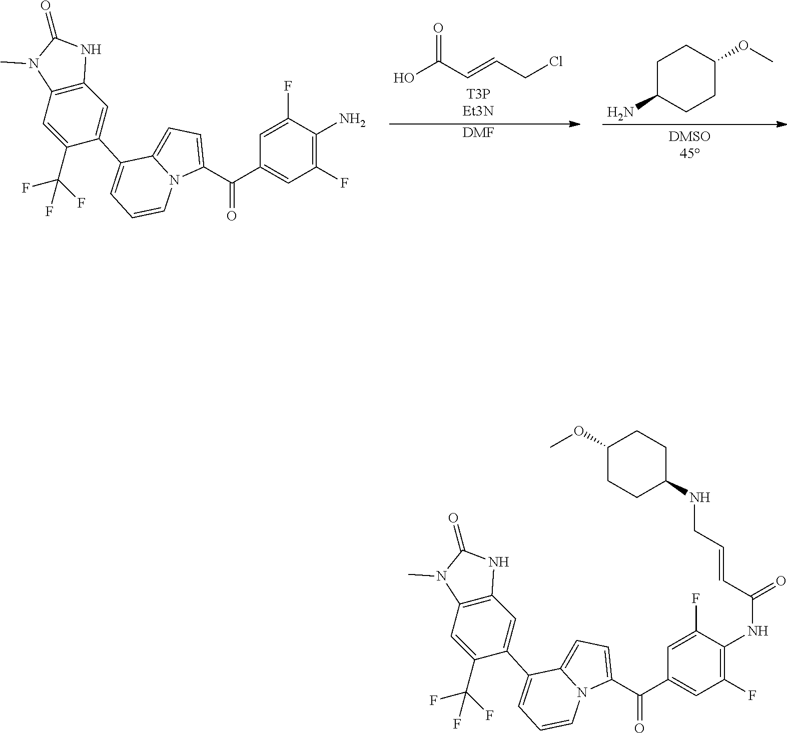

To a solution of crude 5-(3-(4-amino-3,5-difluorobenzoyl)indolizin-8-yl)-1-methyl-6-(trifluoromethyl)-1,3-dihydro-2H-benzo[d]imidazol-2-one (119 mg) and triethyl amine (0.759 mL, 5.57 mmol) in DMF (1.5 mL) was slowly added 0.6 mL of 4-chlorocrotonic acid T3P solution (prepared from (E)-4-chlorobut-2-enoic and T3P (50 wt % in THF)(1 mL/100 mg (4-chlorocrotonic acid))). And additional 4-chlorocrotonic acid T3P solution (0.9 mL) was added thereto (until the starting material almost disappeared). The resulting mixture was diluted with EtOAc, washed with NaHCO₃ aq. H₂O and brine then dried over Na₂SO₄ and evaporated. To the residue was added DMSO (1.5 mL) and trans-4-methoxycyclohexan-1-amine (135 μL), and the mixture was stirred at 45° C. for 2.5 hours. The mixture was purified by preparative reversed-phase HPLC (water:acetonitrile (0.1% formic acid)). Saturated sodium bicarbonate water was added to the purified fractions containing the title compound, followed by extraction with ethyl acetate. The extract was washed with brine and dried over Na₂SO₄. The solvent was evaporated under reduced pressure to obtain (E)-N-(2,6-difluoro-4-(8(1-methyl-2-oxo-6-(trifluoromethyl)-2,3-dihydro-1H-benzo[d]imidazol-5-yl)indolizine-3-carbonyl)phenyl)-4-(((1r,4r)-4-methoxycyclohexyl)amino)but-2-enamide.

### Example 111-1 and 111-2

### (E)-N-(2,6-difluoro-4-(8-(4-fluoro-6-methoxy-1,2-dimethyl-1H-benzo[d]imidazol-5-yl)-1-vinylindolizine-3-carbonyl)phenyl)-44-((1-methylcyclopropyl)amino)but-2-enamide

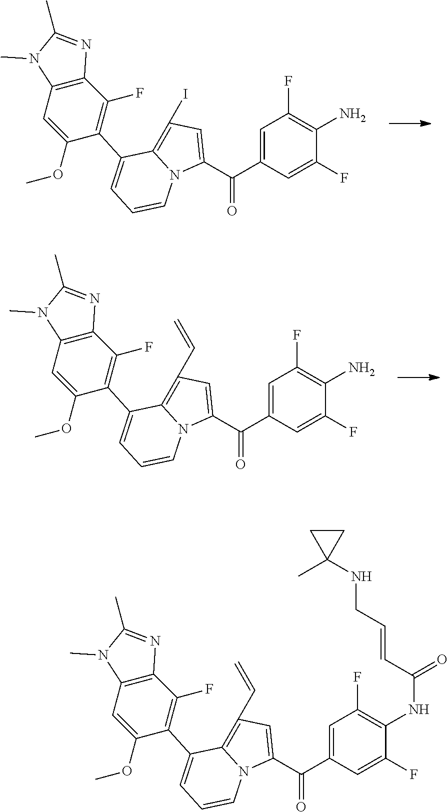 [SMILES: COc1cc2c(nc(C)n2C)c(F)c1-c1cccn2c(C(=O)c3cc(F)c(N)c(F)c3)cc(I)c12 ; C=Cc1cc(C(=O)c2cc(F)c(N)c(F)c2)n2cccc(-c3c(OC)cc4c(nc(C)n4C)c3F)c12 ; C=Cc1cc(C(=O)c2cc(F)c(NC(=O)/C=C/CNC3(C)CC3)c(F)c2)n2cccc(-c3c(OC)cc4c(nc(C)n4C)c3F)c12]

The procedure of Example 111 was performed in a similar manner as Example 87 to give the racemic title compound. The racemate (7.00 mg, 0.0112 mmol) was separated by CHIRALPAK-IC (DAICEL) (hexane-EtOH(0.1% Et₃N)), thereby obtaining the each isomer of the title compound as the 1^{st} elution (Example 111-1) and 2^{nd} elution (Example 111-2).

### Example 112-1 and 112-2

### (E)-N-(2,6-difluoro-4-(6-fluoro-8-(4-methoxy-1,2-dimethyl-6-(trifluoromethyl)-1H-benzo[d]imidazol-5-yl)imidazol[1,2-a]pyridine-3-carbonyl)phenyl)-4-(1-methylcyclopropyl)amino)but-2-enamide

### Step 1

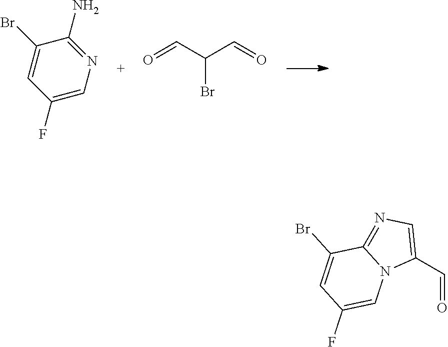 [SMILES: Nc1ncc(F)cc1Br ; O=CC(Br)C=O ; O=Cc1cnc2c(Br)cc(F)cn12]

A solution of 3-bromo-5-fluoropyridin-2-amine (1.66 g) and 2-bromomalonaldehyde (1.97 g) in ethanol (10 mL) and water (10 mL) was stirred at 110° C. for 30 minutes using microwave. The solvent was evaporated under reduced pressure. After dilution with ethyl acetate, the mixture was washed with a saturated sodium bicarbonate solution and a saturated sodium chloride solution, followed by drying over sodium sulfate. The solvent was evaporated under reduced pressure, thereby obtaining crude 8-bromo-6-fluoroimidazo[1,2-a]pyridine-3-carbaldehyde.

### Step 2

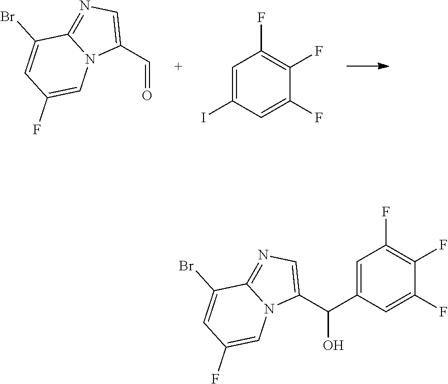 [SMILES: O=Cc1cnc2c(Br)cc(F)cn12 ; Fc1cc(I)cc(F)c1F ; OC(c1cc(F)c(F)c(F)c1)c1cnc2c(Br)cc(F)cn12]

A solution of 1,2,3-trifluoro-5-iodobenzene (7.63 g) in tetrahydrofuran (100 mL) was cooled to −10° C. To the reaction mixture was added isopropylmagnesium chloride-lithium chloride complex (tetrahydrofuran solution (abt. 14%). 28.5 mL). After stirring at −10° C. for 1 hour, crude 8-bromo-6-fluoroimidazo[1,2-a]pyridine-3-carbaldehyde (4.00 g) was added. A reaction was performed at −10° C. for 1 hour. After warming up to room temperature, the reaction mixture was quenched with a saturated ammonium chloride solution. After the mixture was extracted with ethyl acetate, the organic layer was washed with a saturated sodium chloride solution, followed by drying over sodium sulfate. After evaporation, the obtained residue was purified by column chromatography (hexane:ethyl acetate), thereby obtaining (8-bromo-6-fluoroimidazo[1,2-a]pyridin-3-yl)(3,4,5-trifluorophenyl)methanol.

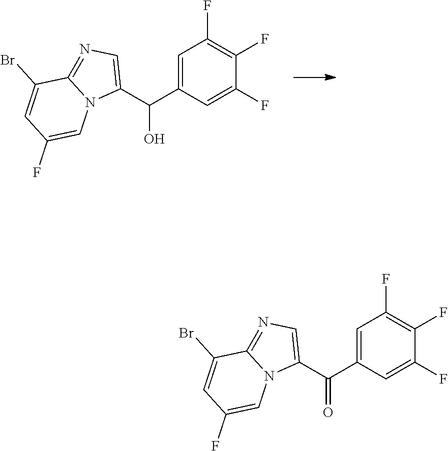 [SMILES: OC(c1cc(F)c(F)c(F)c1)c1cnc2c(Br)cc(F)cn12 ; O=C(c1cc(F)c(F)c(F)c1)c1cnc2c(Br)cc(F)cn12]

A solution of (8-bromo-6-fluoroimidazo[1,2-a]pyridin-3-yl)(3,4,5-trifluorophenyl)methanol (4.6 g) and manganese dioxide (5.33 g) in ethyl acetate (30 mL) was stirred at 60° C. for 14 hours. The insoluble matter was filtered off, followed by evaporating the solvent under reduced pressure. The obtained residue was purified by column chromatography (hexane:ethyl acetate), thereby obtaining (8-bromo-6-fluoroimidazo[1,2-a]pyridin-3-yl)(3,4,5-trifluorophenyl)methanone.

### Step 4

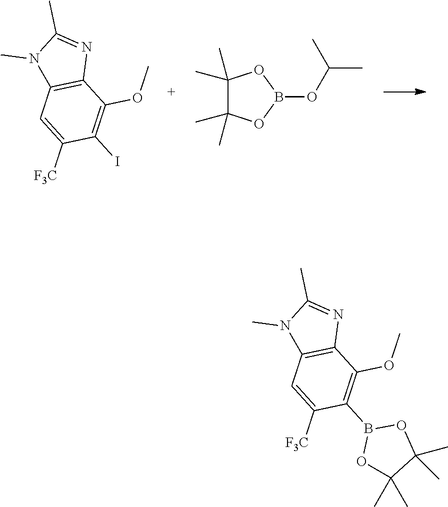

A solution of 5-iodo-4-methoxy-1,2-dimethyl-6-(trifluoromethyl)-1H-benzo[d]imidazole (900 mg) and 2-isopropoxy-4,4,5,5-tetramethyl-1,3,2-dioxaborolane (1.11 g) in tetrahydrofuran (20 mL) was cooled to −10° C. To the reaction mixture was added isopropylmagnesium chloride-lithium chloride complex (tetrahydrofuran solution (abt. 14%). 2.62 mL). A reaction was performed at −10° C. for 1 hour. After warming up to room temperature, the reaction mixture was quenched with a saturated ammonium chloride solution. After the mixture was extracted with ethyl acetate, the organic layer was washed with a saturated sodium chloride solution, followed by drying over sodium sulfate. After evaporation, the obtained residue was purified by column chromatography (hexane:ethyl acetate), thereby obtaining 4-methoxy-1,2-dimethyl-5-(4,4,5,5-tetramethyl-1,3,2-dioxaborolan-2-yl)-6-(trifluoromethyl)-1H-benzo[d]imidazole.

### Step 5

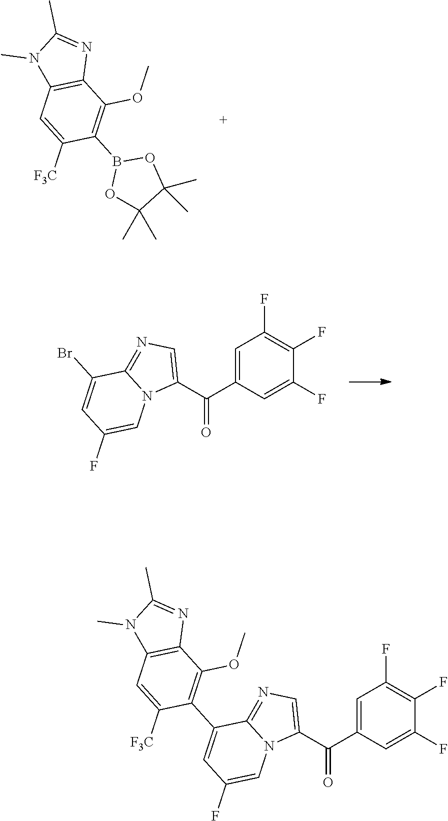

A solution of 4-methoxy-1,2-dimethyl-5-(4,4,5,5-tetramethyl-1,3,2-dioxaborolan-2-yl)-6-(trifluoromethyl)-1H-benzo[d]imidazole (133 mg) obtained in step 4, (8-bromo-6-fluoroimidazo[1,2-a]pyridin-3-yl)(3,4,5-trifluorophenyl)methanone (132 mg) obtained in step 3. SPhos Pd G3 (37.7 mg), and 2.0 M K₃PO₄ in water (356 μL) in tetrahydrofuran (7.0 mL) was stirred at 100° C. for 5 hours using microwave irradiation. After dilution with ethyl acetate and water, the mixture was extracted with ethyl acetate. The combined organic layer was washed with water and a saturated sodium chloride solution, followed by drying over sodium sulfate. After evaporation, the obtained residue was purified by column chromatography (hexane:acetone), thereby obtaining (6-fluoro-8-(4-methoxy-1,2-dimethyl-6-(trifluoromethyl)-1H-benzo[d]imidazol-5-yl)imidazo[1,2-a]pyridin-3-yl)(3,4,5-trifluorophenyl)methanone.

### Step 6

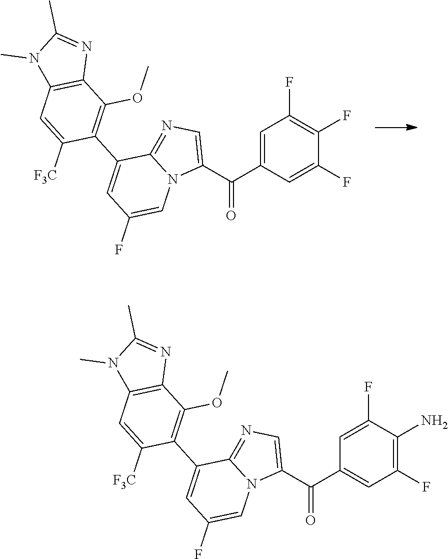 [SMILES: COc1c(-c2cc(F)cn3c(C(=O)c4cc(F)c(F)c(F)c4)cnc23)c(C(F)(F)F)cc2c1nc(C)n2C ; COc1c(-c2cc(F)cn3c(C(=O)c4cc(F)c(N)c(F)c4)cnc23)c(C(F)(F)F)cc2c1nc(C)n2C]

A solution of (6-fluoro-8-(4-methoxy-1,2-dimethyl-6-(trifluoromethyl)-1H-benzo[d]imidazol-5-yl)imidazol[1,2-a]pyridin-3-yl)(3,4,5-trifluorophenyl)methanone (63.0 mg) obtained in step 5 and 28% ammonia solution (2.0 mL) in 1,4-dioxane (2.0 mL) was stirred at 140° C. for 8 hours using microwave irradiation. After dilution with ethyl acetate and water, the mixture was extracted with ethyl acetate. The combined organic layer was washed with a saturated sodium chloride solution, followed by drying over sodium sulfate. After evaporation, the obtained residue was purified by column chromatography (chloroform:ethanol), thereby obtaining (4-amino-3,5-difluorophenyl)(6-fluoro-8-(4-methoxy-1,2-dimethyl-6-(trifluoromethyl)-1H-benzo[d]imidazol-5-yl)imidazo[1,2-a]pyridin-3-yl)methanone.

### Step 7

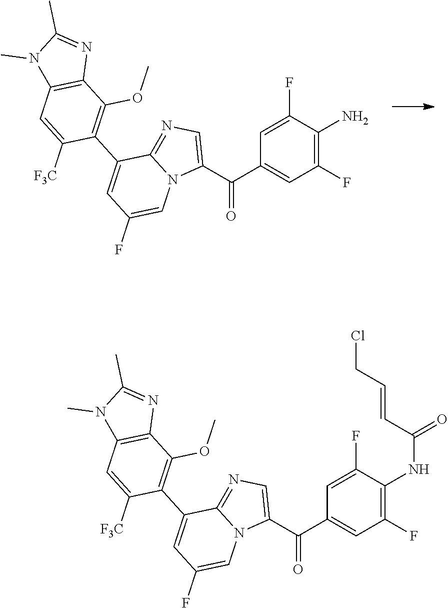 [SMILES: COc1c(-c2cc(F)cn3c(C(=O)c4cc(F)c(N)c(F)c4)cnc23)c(C(F)(F)F)cc2c1nc(C)n2C ; COc1c(-c2cc(F)cn3c(C(=O)c4cc(F)c(NC(=O)/C=C/CCl)c(F)c4)cnc23)c(C(F)(F)F)cc2c1nc(C)n2C]

(E)-4-chlorobut-2-enoic acid (12.3 mg) and 1-propanephosphonic acid anhydride cyclic trimer (a 1.7M THF solution, 123 μL) were added to a solution of (4-amino-3,5-difluorophenyl)(6-fluoro-8-(4-methoxy-1,2-dimethyl-6-(trifluoromethyl)-1H-benzo[d]imidazol-5-yl)imidazol[1,2-a]pyridin-3-yl)methanone (42.0 mg) obtained in step 6 in DMF (0.8 mL), followed by stirring at room temperature for 20 minutes. Triethylamine (44.0 μL) was added to the reaction mixture. A reaction was performed at room temperature for 2 hours. After water was added to the reaction mixture, extraction was performed with ethyl acetate, followed by washing the organic layer with a saturated sodium chloride solution. The washed organic layer was dried over sodium sulfate. After evaporation, the obtained residue was purified by column chromatography (chloroform:ethanol), thereby obtaining (E)-4-chloro-N-(2,6-difluoro-4-(6-fluoro-8-(4-methoxy-1,2-dimethyl-6-(trifluoromethyl)-1H-benzo[d]imidazo-5-yl)imidazo[1,2-a]pyridine-3-carbonyl)phenyl)but-2-enamide.

### Step 8

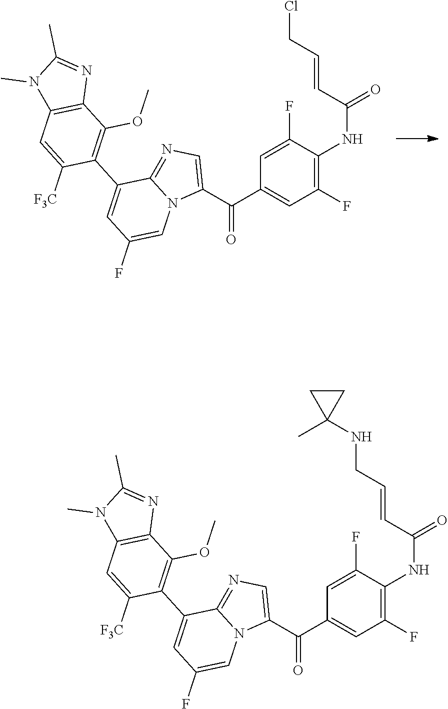 [SMILES: COc1c(-c2cc(F)cn3c(C(=O)c4cc(F)c(NC(=O)/C=C/CCl)c(F)c4)cnc23)c(C(F)(F)F)cc2c1nc(C)n2C ; COc1c(-c2cc(F)cn3c(C(=O)c4cc(F)c(NC(=O)/C=C/CNC5(C)CC5)c(F)c4)cnc23)c(C(F)(F)F)cc2c1nc(C)n2C]

1-methylcyclopropan-1-amine (17.2 μL) and potassium iodide (20.1 mg) were added to a solution of (E)-4-chloro-N-(2,6-difluoro-4-(6-fluoro-8-(4-methoxy-1,2-dimethyl-6-(trifluoromethyl)-1H-benzo[d]imidazol-5-yl)imidazo[1,2-a]pyridine-3-carbonyl)phenyl)but-2-enamide (27.0 mg) in DMSO (900 μL). A reaction was performed at room temperature for 2 hours. After water was added to the reaction mixture, extraction was performed with ethyl acetate. The combined organic layer was washed with a saturated sodium chloride solution. After the washed layer was dried over sodium sulfate, the solvent was evaporated under reduced pressure. The obtained residue was purified by column chromatography (chloroform:ethanol), thereby obtaining the racemic title compound (21.0 mg). The racemate (18.8 mg) was dissolved in EtOH and purified by preparative HPLC (CHIRALPAK IC, hexane-EtOH (0.1% Et₃N)) to afford (E)-N-(2,6-difluoro-4-(6-fluoro-8-(4-methoxy-1,2-dimethyl-6-(trifluoromethyl)-1H-benzo[d]imidazol-5-yl)imidazo[1,2-a]pyridine-3-carbonyl)phenyl)-4-((1-methylcyclopropyl)amino)but-2-enamide (Example 112-1, single isomer, 1st-eluting isomer) and (E)-N-(2,6-difluoro-4-(6-fluoro-8-(4-methoxy-1,2-dimethyl-6-(trifluoromethyl)-1H-benzo[d]imidazol-5-yl)imidazo[1,2-a]pyridine-3-carbonyl)phenyl)-4-((1-methylcyclopropyl)amino)but-2-enamide (Example 112-2, single isomer, 2nd-eluting isomer).

### Example 113

(E)-N-(2,6-difluoro-4-(8-(4-fluoro-6-methoxy-1-(2-methoxyethyl)-2-methyl-1H-benzo[d]imidazol-5-vi)indolizine-3-carbonyl)phenyl)-4-((1-methylcyclopropyl)amino)but-2-enamide

### Step 1

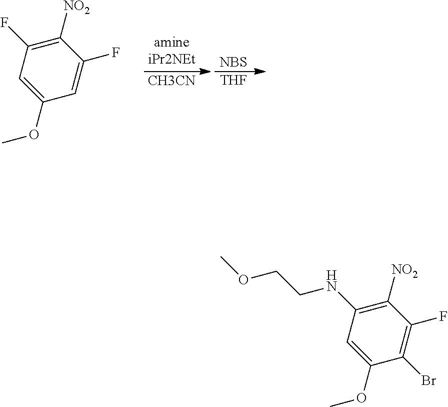

2-Methoxyethylamine (3.31 mL, 38 mmol) and N,N-diisopropylethylamine (13.3 mL, 76.1 mmol) were slowly added to a solution of 1,3-difluoro-5-methoxy-2-nitrobenzene (6.54 g. 34.6 mmol) in acetonitrile (150 mL) at room temperature. After stirring at room temperature overnight, water and EtOAc were added to the resulting mixture. The organic layer was separated, and washed with brine, followed by drying over sodium sulfate. After evaporation, to the obtained residue was added THF (100 mL), and NBS (6.16 g. 34.6 mmol) was added thereto at 0° C. After stirring for 10 minutes, the precipitates were collected by filtration and washed with diisopropylether to obtain 4-bromo-3-fluoro-5-methoxy-N-(2-methoxyethyl)-2-nitroaniline (4.67 g, 42%). The filtrates were diluted with EtOAc and washed with saturated NaHCO₃ aq. and brine, dried over Na₂SO₄ and evaporated. The residue was diluted with diisopropylether, and collected by filtration to obtain 4-bromo-3-fluoro-5-methoxy-N-(2-methoxyethyl)-2-nitroaniline.

### Step 2

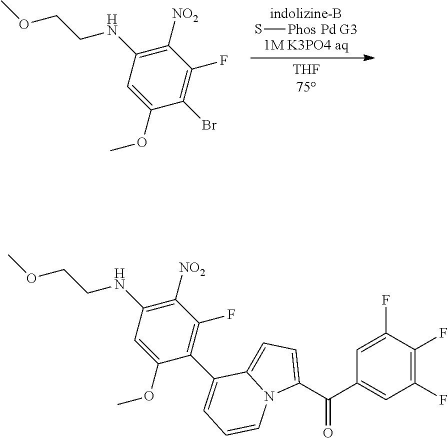

A mixture of 4-bromo-3-fluoro-5-methoxy-N-(2-methoxyethyl)-2-nitroaniline (4.49 g, 10 mmol, 72 wt %), (8-(5,5-dimethyl-1,3,2-dioxaborinan-2-yl)indolizin-3-yl)(3,4,5-trifluorophenyl)methanone (5.00 g, 12.9 mmol) and S-Phos Pd G3 (360 mg) in THF (70 mL) and 1M K₃PO₄ aq. (15 mL, 15.0 mmol) was evacuated and N₂ back-filled before heating to 75° C. for 1 hour. After cooling, the reaction mixture was diluted with EtOAc and brine. The organic layer was separated and dried over sodium sulfate. After evaporation, the resulting residue was suspended with diisopropyl ether and collected by filtration to obtain (8-(2-fluoro-6-methoxy-4-((2-methoxyethyl)amino)-3-nitrophenyl)indolizin-3-yl)(3,4,5-trifluorophenyl)methanone.

### Step 3

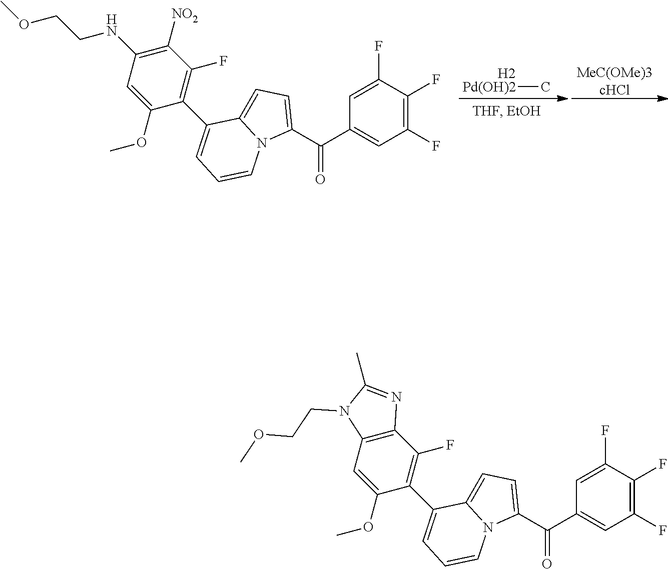

To a solution of (8-(2-fluoro-6-methoxy-4-((2-methoxyethyl)amino)-3-nitrophenyl)indolizin-3-yl)(3,4,5-trifluorophenyl)methanone (1.03 g, 2.00 mmol) in THF (11.3 mL) and ethanol (11.3 mL) was added palladium hydroxide on carbon (520 mg, 20 wt. % loading (dry basis)). The mixture was stirred at room temperature overnight under hydrogen atmosphere. After nitrogen purging, the mixture was filtered, rinsing with EtOAc and MeOH. The filtrate was evaporated, and to the residue was added trimethyl orthoacetate (12 mL) and conc. HCl aq. (0.1 mL). After adding sat. NaHCO₃ aq. and EtOAc, the organic layer was separated, dried over Na₂SO₄, and evaporated to obtain (8-(4-fluoro-6-methoxy-1-(2-methoxyethyl)-2-methyl-1H-benzo[d]imidazol-5-yl)indolizin-3-yl)(3,4,5-trifluorophenyl)methanone.

### Step 4

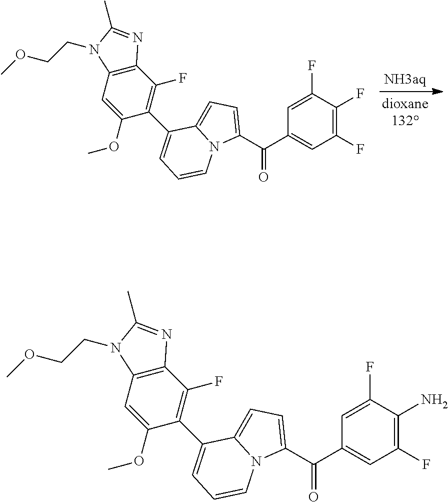 [SMILES: COCCn1c(C)nc2c(F)c(-c3cccn4c(C(=O)c5cc(F)c(F)c(F)c5)ccc34)c(OC)cc21 ; COCCn1c(C)nc2c(F)c(-c3cccn4c(C(=O)c5cc(F)c(N)c(F)c5)ccc34)c(OC)cc21]

The mixture of (8-(4-fluoro-6-methoxy-1-(2-methoxyethyl)-2-methyl-1H-benzo[d]imidazol-5-yl)indolizin-3-yl)(3,4,5-trifluorophenyl)methanone (1.05 g). 1,4-dioxane (6.0 mL) and 28-30% NH₃ aq. (12 mL) was heated at 132° C. by microwave irradiation for 14 hours. After cooling, the mixture was diluted with EtOAc, washed with brine, then dried over Na₂SO₄ and evaporated. The residue was purified by column chromatography on silica gel (hexane-EtOAc/EtOH(4/1)) to obtain (4-amino-3,5-difluorophenyl)(8-(4-fluoro-6-methoxy-1-(2-methoxyethyl)-2-methyl-1H-benzo[d]imidazol-5-yl)indolizin-3-yl)methanone.

### Step 5

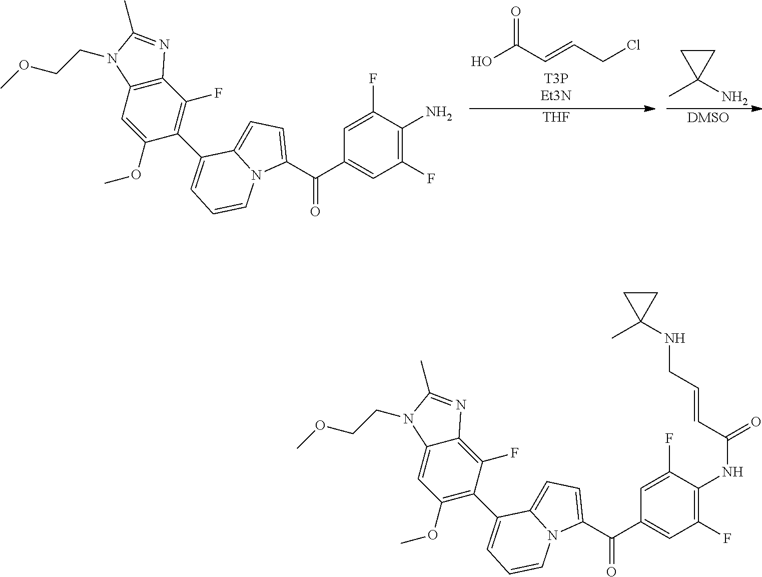

To the mixture of (4-amino-3,5-difluorophenyl)(8-(4-fluoro-6-methoxy-1-(2-methoxyethyl)-2-methyl-1H-benzo[d]imidazol-5-yl)indolizin-3-yl)methanone (76.2 mg, 0.150 mmol), (E)-4-chlorobut-2-enoic acid (23.5 mg, 0.195 mmol) and T3P (50 wt % in THF, 0.468 mL) in THF (1 mL) was added triethylamine (0.163 mL, 1.20 mmol). After being stirred at room temperature for 30 minutes, the resulting mixture was diluted with EtOAc, washed with NaHCO₃ aq., H₂O and brine then dried over Na₂SO₄ and evaporated. To the residue was added DMSO (0.9 mL), 1-methylcyclopropan-1-amine (0.143 mL, 1.80 mmol), and the mixture was stirred at 40° C. for 3 hours. The mixture was purified by preparative HPLC (water:acetonitrile (0.1% formic acid)) to give (E)-N-(2,6-difluoro-4-(8-(4-fluoro-6-methoxy-1-(2-methoxyethyl)-2-methyl-1H-benzo[d]imidazol-5-yl)indolizine-3-carbonyl)phenyl)-4-((1-methylcyclopropyl)amino)but-2-enamide.

### Example 114

### (E)-N-(2,6-difluoro-4-(8-(5-fluoro-7-methoxy-1-methyl-4-oxo-1,4-dihydroquinazolin-6-yl)indolizine-3-carbonyl)phenyl-4-((1-methylcyclopropyl)amino)but-2-enamide

### Step 1

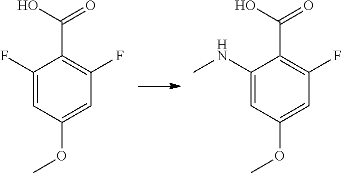

Methylamine (2M THF solution, 10 mL, 19.9 mmol) was added to a solution of 2,6-difluoro-4-methoxybenzoic acid (500 mg, 2.66 mmol) in DMSO (8.9 mL). The reaction mixture was heated at 130° C. by microwave irradiation for 4 hours. After cooling, the reaction mixture was alkalinized with NaOH aq. and washed with EtOAc. The aqueous layer was acidified with HCl aq. and extracted with EtOAc. The organic layer was dried over Na₂SO₄ and evaporated, thereby obtaining crude 2-fluoro-4-methoxy-6-(methylamino)benzoic acid.

### Step 2

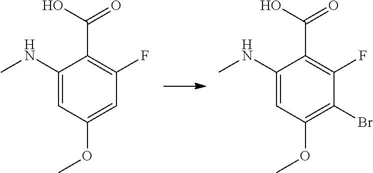

N-Bromosuccinimide (249 mg, 1.40 mmol) was added to a solution of crude 2-fluoro-4-methoxy-6-(methylamino)benzoic acid (266 mg, 1.34 mmol) in THF (6.7 mL).

After stirring at room temperature for 30 minutes, the mixture was diluted with EtOAc, washed with H₂O, 20% Na₂S₂O₃ aq. and brine then dried over Na₂SO₄ and evaporated, thereby obtaining crude 3-bromo-2-fluoro-4-methoxy-6-(methylamino)benzoic acid.

### Step 3

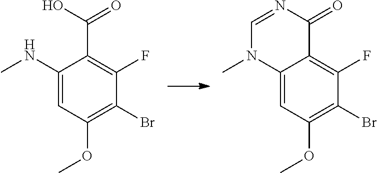 [SMILES: CNc1cc(OC)c(Br)c(F)c1C(=O)O ; COc1cc2c(c(F)c1Br)c(=O)ncn2C]

A mixture of crude 3-bromo-2-fluoro-4-methoxy-6-(methylamino)benzoic acid (341 mg, 1.23 mmol), ammonium acetate (952 mg, 12.3 mmol) and triethyl orthoformate (4.1 mL, 24.5 mmol) in MeOH (4.1 μm) was stirred at 100° C. for 6 hours. After cooling, the precipitated solid was collected by filtration, rinsing with H₂O, and vacuumed at 50° C., thereby obtaining 6-bromo-5-fluoro-7-methoxy-1-methylquinazolin-4(1H)-one.

### Step 4

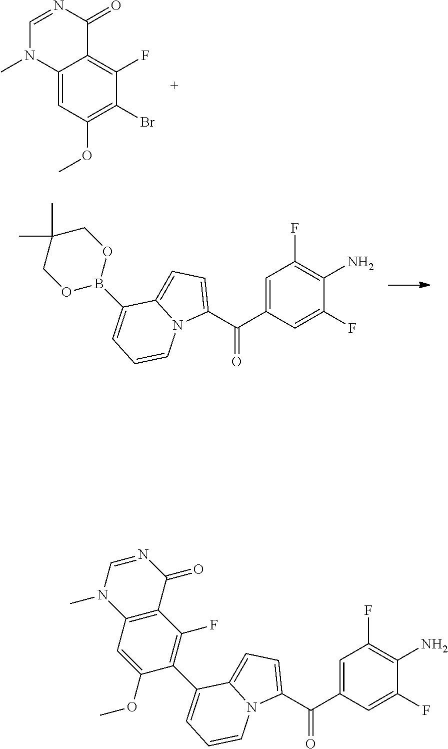

A mixture of 6-bromo-5-fluoro-7-methoxy-1-methylquinazolin-4(1H)-one (100 mg, 0.348 mmol), (4-amino-3,5-difluorophenyl)(8-(5,5-dimethyl-1,3,2-dioxaborinan-2-yl)indolizin-3-yl)methanone (201 mg, 0.522 mmol) and SPhos Pd G3 (25 mg, 35 μmol) in toluene (1.7 mL) and 2M K₃PO₄ aq. (392 μL, 0.784 mmol) was evacuated and N₂ back-filled before heating at 80° C. for 3 hours. After cooling. EtOAc was added to the reaction mixture then collected by filtration, rinsing with EtOAc and H₂O, and vacuumed at 50° C., thereby obtaining crude 6-(3-(4-amino-3,5-difluorobenzoyl)indolizin-8-yl)-5-fluoro-7-methoxy-1-methylquinazolin-4(1H)-one.

### Step 5-6

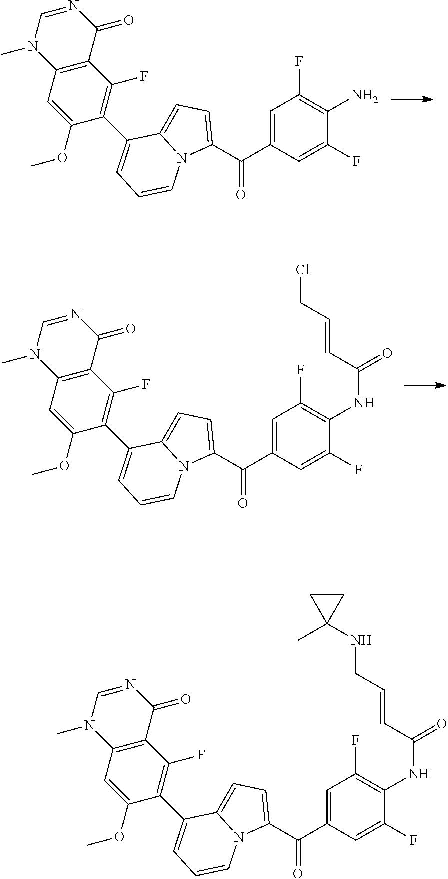 [SMILES: COc1cc2c(c(F)c1-c1cccn3c(C(=O)c4cc(F)c(N)c(F)c4)ccc13)c(=O)ncn2C ; COc1cc2c(c(F)c1-c1cccn3c(C(=O)c4cc(F)c(NC(=O)/C=C/CCl)c(F)c4)ccc13)c(=O)ncn2C ; COc1cc2c(c(F)c1-c1cccn3c(C(=O)c4cc(F)c(NC(=O)/C=C/CNC5(C)CC5)c(F)c4)ccc13)c(=O)ncn2C]

The procedure was performed in the same manner as Example 113 (step 5) to afford the title compound.

### Example 115

(E)-N-(4-(1-(1,2-dimethyl-6-(trifluoromethyl)-1H-benzo[d]imidazol-5-yl)pyrrolo[1,2-a]pyrazine-6-carbonyl)-2,6-difluorophenyl)-4-(((1r,4r)-4-methoxycyclohexyl)amino)but-2-enamide

### Step 1

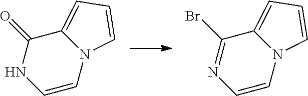 [SMILES: O=c1[nH]ccn2cccc12 ; Brc1nccn2cccc12]

To a mixture of pyrrolo[1,2-a]pyrazin-1(2H)-one (2.60 g. 19.4 mmol) in MeCN (27.7 mL) was added phosphoryl bromide (3.94 mL, 38.8 mmol) at ambient temperature and heated to 80° C. for 6 hours. The reaction mixture was slowly poured onto ice cold water, neutralized with aqueous ammonium hydroxide, and extracted with EtOAc. and washed successively with H₂O then brine, dried over sodium sulfate, and evaporated. The residue was purified by column chromatography on silica gel (EtOAc-hexane), thereby obtaining 1-bromopyrrolo[1,2-a]pyrazine.

### Step 2

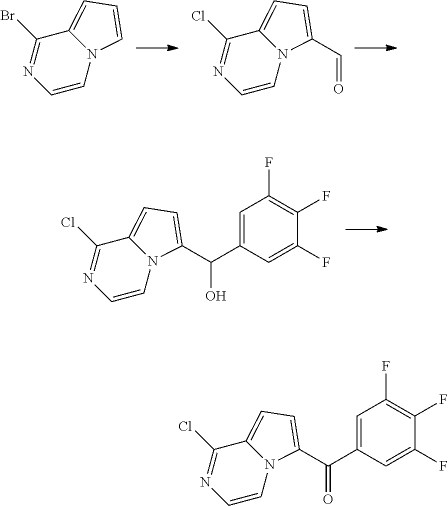 [SMILES: Brc1nccn2cccc12 ; O=Cc1ccc2c(Cl)nccn12 ; OC(c1cc(F)c(F)c(F)c1)c1ccc2c(Cl)nccn12 ; O=C(c1cc(F)c(F)c(F)c1)c1ccc2c(Cl)nccn12]

Phosphoryl trichloride (1.07 mL, 11.7 mmol) was added to DMF (1.81 mL, 23.4 mmol) at 0° C. The reaction mixture was stirred at ambient temperature for 50 minutes. This solution was added to a separate solution of 1-bromopyrrolo[1,2-a]pyrazine (1.77 g. 8.98 mmol) in DMF (12.8 mL) at 0° C. The reaction mixture was stirred at ambient temperature for 2 hours. The reaction mixture was slowly poured onto ice cold water, neutralized with a saturated aqueous NaHCO₃, and extracted with EtOAc, and washed successively with H₂O then brine, dried over sodium sulfate, and evaporated. The residue was used for the next reaction.

A solution of 1,2,3-trifluoro-5-iodo-benzene (1.34 mL, 10.8 mmol) in tetrahydrofuran (30.0 mL) was cooled to 0° C. To the reaction mixture was added isopropylmagnesium chloride-lithium chloride complex (tetrahydrofuran solution (abt. 14%). 12.4 mL, 16.2 mmol). After stirring at ambient temperature for 10 minutes. 1-chloropyrrolo[1,2-a]pyrazine-6-carbaldehyde (2.02 g, 8.98 mmil) was added to the reaction mixture at 0° C. A reaction was performed at ambient temperature for 1 hour. To the resulting mixture was added saturated aqueous ammonium chloride, and extracted with EtOAc, and washed successively with H₂O then brine, dried over sodium sulfate, and evaporated. The residue was used for the next reaction.

Manganese dioxide (7.79 g, 89.6 mmol) was added to a solution of (1-chloropyrrolo[1,2-a]pyrazin-6-yl)(3,4,5-trifluorophenyl)methanol (2.80 g. 8.96 mmol) in EtOAc (44.8 mL). After the mixture was stirred at 65° C. for 2.5 days, the mixture was cooled to ambient temperature. EtOAc was added to the reaction mixture, and insoluble matter was filtered off through celite pad, and the mixture was extracted with EtOAc and H₂O, and washed successively with H₂O then brine, dried over sodium sulfate, and evaporated. The residue was purified by column chromatography on silica gel (EtOAc-hexane), thereby obtaining (1-chloropyrrolo[1,2-a]pyrazin-6-yl)(3,4,5-trifluorophenyl)methanone.

### Step 3

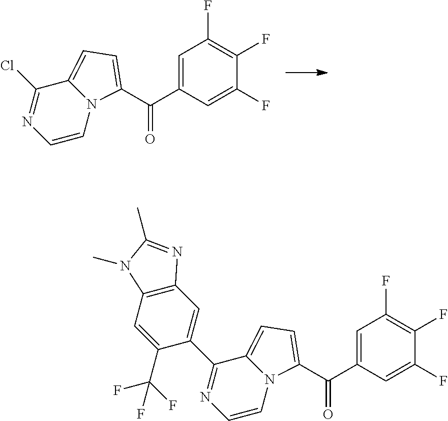 [SMILES: O=C(c1cc(F)c(F)c(F)c1)c1ccc2c(Cl)nccn12 ; Cc1nc2cc(-c3nccn4c(C(=O)c5cc(F)c(F)c(F)c5)ccc34)c(C(F)(F)F)cc2n1C]

To a mixture of (1-chloropyrrolo[1,2-a]pyrazin-6-yl)(3,4,5-trifluorophenyl)methanone (300 mg, 0.966 mmol), 1,2-dimethyl-5-(4,4,5,5-tetramethyl-1,3,2-dioxaborolan-2-yl)-6-(trifluoromethyl)benzimidazole (573 mg, 1.45 mmol) and Pd(PPh₃)₄ (112 mg, 0.0966 mmol) in 1,2-dimethoxyethane (4.80 mL) was added 2M aqueous sodium carbonate solution (966 μL, 1.93 mmol). After reacting by microwave apparatus at 120° C. for 1 hour, the mixture was diluted with EtOAc and water, extracted with EtOAc, and washed successively with H₂O then brine, dried over sodium sulfate, and evaporated. The residue was purified by column chromatography on silica gel (acetone-hexane), thereby obtaining (1-(1,2-dimethyl-6-(trifluoromethyl)-1H-benzo[d]imidazol-5-yl)pyrrolo[1,2-a]pyrazin-6-yl)(3,4,5-trifluorophenyl)methanone.

### Step 4

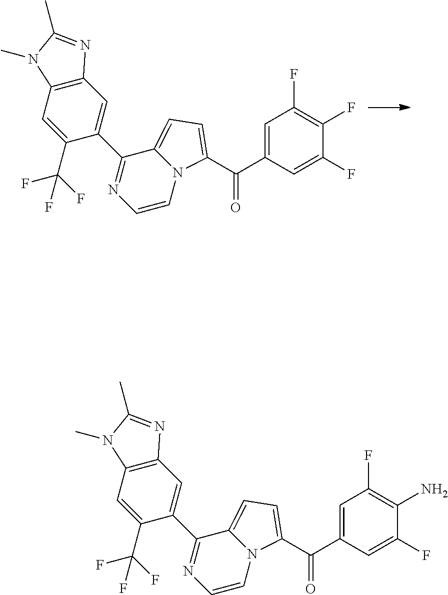 [SMILES: Cc1nc2cc(-c3nccn4c(C(=O)c5cc(F)c(F)c(F)c5)ccc34)c(C(F)(F)F)cc2n1C ; Cc1nc2cc(-c3nccn4c(C(=O)c5cc(F)c(N)c(F)c5)ccc34)c(C(F)(F)F)cc2n1C]

To a mixture of (1-(1,2-dimethyl-6-(trifluoromethyl)-1H-benzo[d]imidazol-5-yl)pyrrolo[1,2-a]pyrazin-6-yl)(3,4,5-trifluorophenyl)methanone (150 mg, 0.310 mmol) in 1,4-dioxane (9.00 mL) was added ammonium hydroxide (9.00 mL, 76.0 mmol). After reacting by microwave apparatus at 135° C. for 4 hours, the mixture was diluted with EtOAc and water, extracted with EtOAc, and washed successively with H₂O then brine, dried over sodium sulfate, and evaporated, thereby obtaining crude (4-amino-3,5-difluorophenyl)(1-(1,2-dimethyl-6-(trifluoromethyl)-1H-benzo[d]imidazol-5-yl)pyrrolo[1,2-a]pyrazin-6-yl)methanone.

### Step 5

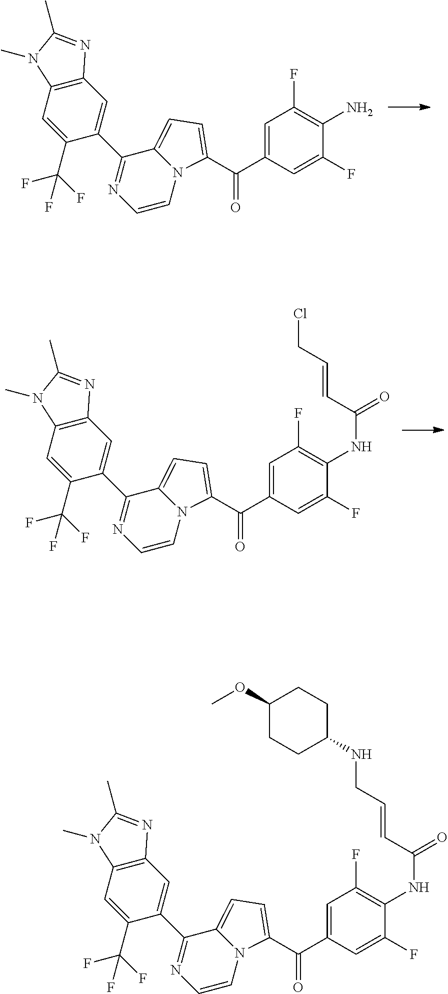 [SMILES: Cc1nc2cc(-c3nccn4c(C(=O)c5cc(F)c(N)c(F)c5)ccc34)c(C(F)(F)F)cc2n1C ; Cc1nc2cc(-c3nccn4c(C(=O)c5cc(F)c(NC(=O)/C=C/CCl)c(F)c5)ccc34)c(C(F)(F)F)cc2n1C ; CO[C@H]1CC[C@H](NC/C=C/C(=O)Nc2c(F)cc(C(=O)c3ccc4c(-c5cc6nc(C)n(C)c6cc5C(F)(F)F)nccn34)cc2F)CC1]

A solution of (4-amino-3,5-difluorophenyl)(1-(1,2-dimethyl-6-(trifluoromethyl)-1H-benzo[d]imidazol-5-yl)pyrrolo[1,2-a]pyrazin-6-yl)methanone (50.0 mg, 0.103 mmol), 4-chlorocrotonic acid (14.9 mg, 0.124 mmol) and propylphosphonic anhydride solution (50 wt % in THF. 193 μL, 0.309 mmol) in DMF (1.00 mL) was stirred at ambient temperature for 10 minutes. Then triethylamine (85.8 μL, 0.618 mmol) was added thereto, and the mixture was stirred at ambient temperature for 30 minutes. To the resulting mixture was added saturated aqueous NaHCO₃, and extracted with EtOAc, and washed successively with H₂O then brine, dried over sodium sulfate, and evaporated. The residue was used for the next reaction.

A solution of (E)-4-chloro-N-(4-(1-(1,2-dimethyl-6-(trifluoromethyl)-1H-benzo[d]imidazol-5-yl)pyrrolo[1,2-a]pyrazine-6-carbonyl)-2,6-difluorophenyl)but-2-enamide (60.6 mg, 0.103 mmol), trans-4-methoxycyclohexanamine hydrochloride (34.2 mg, 0.206 mmol), KI (51.3 mg, 0.309 mmol) and K₃PO₄ (87.5 mg, 0.412 mmol) in DMSO (1.00 mL) was stirred at 35° C. for 2 hours. The resulting mixture was purified by preparative HPLC (water:acetonitrile (0.1% formic acid)) to give the title compound.

### Example 116

### mono(5-(1-(3,5-difluoro-4-((E)-4-(((S)-tetrahydrofuran-3-yl)ammonio)but-2-enamido)benzoyl)-1H-indol-4-yl)-4-methoxy-1,2-dimethyl-6-(trifluoromethyl)-1H-benzo[d]imidazol-3-ium) mono(2,2,2-trifluoroacetate)

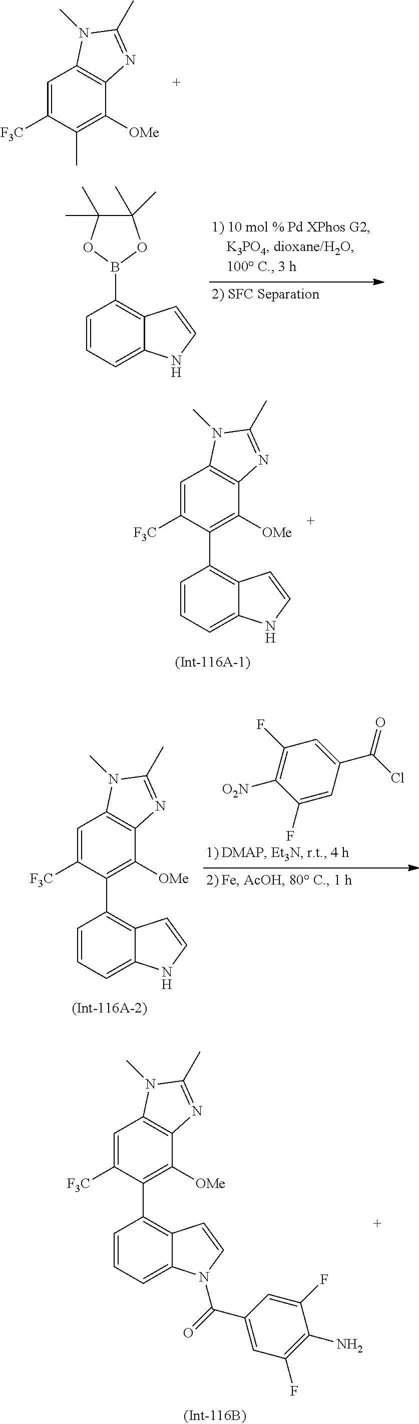

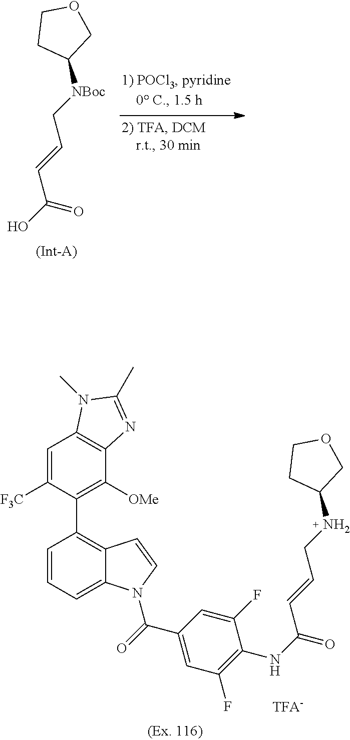

### Step 1

A 25 mL round bottom flask with stir bar was charged with 5-iodo-4-methoxy-1,2-dimethyl-6-(trifluoromethyl)-1H-benzo[d]imidazole (3 g. 8.11 mmol), 4-(4,4,5,5-tetramethyl-1,3,2-dioxaborolan-2-yl)-1H-indole (2.365 g, 9.73 mmol), potassium phosphate tribasic (518 mg, 2.443 mmol), and SPhos Pd G2 (584 mg, 0.811 mmol). 1,4-Dioxane (32.4 ml) and water (8.11 ml) were added via syringe, and the reaction flask was fitted with a septum. The reaction was sparged with N₂ for 15 min and placed in a preheated (100° C.) aluminum block. The reaction was allowed to stir for 3 hours. After 3 hours. TLC analysis indicated that the reaction was complete. The reaction was cooled to room temperature and quenched with water (5 mL). The reaction was poured into a separatory funnel containing 30 mL EtOAc and 30 mL water. The funnel was stoppered, shaken, and the layers were separated. The aqueous phase was washed 2 additional times with EtOAc (30 mL each). The combined organics were washed with brine (50 mL), dried over sodium sulfate, filtered, and concentrated. The residue was purified by column chromatography on silica gel (25 to 75% EtOac in hexanes). 5-(1H-indol-4-yl)-4-methoxy-1,2-dimethyl-6-(trifluoromethyl)-1H-benzo[d]imidazole (Int-116A) was obtained. The atropisomers of Int-116A were separated by preparatory supercritical fluid chromatography (Lux-2, 21×250 mm. 5 μM column. 25% MeOH w/0.1% NH₄OH modifier). The second eluting peak (Int-116A-2) was taken forward as the active atropisomer.

### Step 2

A vial with stir bar was charged with 3,5-difluoro-4-nitrobenzoic acid (90 mg, 0.441 mmol) and placed under nitrogen. Dry dichloromethane (1.2 mL), pyridine (59.4 dl, 0.735 mmol), and 2 drops of DMF were added via syringe. Oxalyl chloride (38.6 μl, 0.441 mmol) was added via syringe and bubbling was observed. The reaction was stirred for 1 h. A separate vial with stir bar was charged with 541H-indol-4-yl)-4-methoxy-1,2-dimethyl-6-(trifluoromethyl)-1H-benzo[d]imidazole (Int-116A-2) (132 mg, 0.367 mmol). 4-dimethylaminopyridine (4.49 mg, 0.037 mmol), triethylamine (102 μl. 0.735 mmol), and dichloromethane (1.2 mL). The solution of acid chloride was added to this solution, and the reaction was stirred at r.t. for 18 hours. After 18 hours, water (10 mL) was added, and the contents were transferred to a separatory funnel. The layers were shaken and separated. The aqueous phase was washed with DCM (2×10 mL). The combined organics were washed with brine (20 mL), dried over MgSO₄, filtered, and concentrated. The residue was purified by column chromatography on silica gel (0% to 50% EtOAc in hexanes). (3,5-difluoro-4-nitrophenyl)(4-(4-methoxy)-1,2-dimethyl-6-(trifluoromethyl)-1H-benzo[d]imidazol-5-yl)-1H-indol-1-yl)methanone was isolated. [M+H]⁺ m/z: 545.

A vial with stir bar was charged with (3,5-difluoro-4-nitrophenyl)(4-(4-methoxy-1,2-dimethyl-6-(trifluoromethyl)-1H-benzo[d]imidazol-5-yl)-1H-indol-1-yl)methanone (182 mg, 0.334 mmol) and iron powder (187 mg, 3.34 mmol). Acetic acid (3.343 mL) was added, and the vial was fitted with a septum. The reaction was heated for 1 h at 60° C. in an aluminum block. After 1 hour, the blood red reaction mixture was cooled to room temperature and diluted with EtOAc. The mixture was filtered through celite and concentrated. The residue was purified by column chromatography on silica gel (33% acetone in hexanes) to give (4-amino-3,5-difluorophenyl)(4-(4-methoxy-1,2-dimethyl-6-(trifluoromethyl)-1H-benzo[d]imidazol-5-yl)-1H-indol-1-yl)methanone (Int-116B). [M+H]⁺ m/z: 515.

### Step 3

A vial with stir bar was charged with (4-amino-3,5-difluorophenyl)(4-(4-methoxy-1,2-dimethyl-6-(trifluoromethyl)-1H-benzo[d]imidazol-5-yl)-1H-indol-1-yl)methanone (62 mg, 0.121 mmol) and (S,E)-4-((tert-butoxycarbonyl)(tetrahydrofuran-3-yl)amino)but-2-enoic acid (Int-A below) (131 mg, 0.482 mmol). The vial was fitted with a septum and placed under nitrogen. Pyridine (1.2 mL) was added via syringe, and the reaction was cooled to 0° C. Phosphorus oxychloride (44.9 μl. 0.482 mmol) (IM solution in pyridine) was added via syringe, and the reaction was warmed to room temperature over 1.5 h. After LCMS analysis indicated reaction was complete, water (2 mL) was added followed by EtOAc (2 mL). The layers were transferred to a separatory funnel, the funnel was shaken, and the layers were separated. The aqueous layer was washed with EtOAc (2×2 mL). The combined organics were dried over MgSO₄, filtered, and concentrated. The crude reaction mixture was purified via silica gel chromatography (33% to 75% EtOAc in hexanes) to obtain tert-butyl ((E)-4-((2,6-difluoro-4-(4-(4-methoxy-1,2-dimethyl-6-(trifluoromethyl)-1H-benzo[d]imidazol-5-yl)-1H-indole-1-carbonyl)phenyl)amino)-4-oxobut-2-en-1-yl)((S)-tetrahydrofuran-3-yl)carbamate. [M+H]⁺ m/z: 768.

Tert-butyl ((E)-4-((2,6-difluoro-4-(4-(4-methoxy-1,2-dimethyl-6-(trifluoromethyl)-1H-benzo[d]imidazol-5-yl)-1H-indole-1-carbonyl)phenyl)amino)-4-oxobut-2-en-1-yl)((S)-tetrahydrofuran-3-yl)carbamate (49 mg, 0.064 mmol) was placed in a vial with stir bar and dissolved in DCM (425 μl). TFA (213 μl) was added dropwise, and the reaction was allowed to stir for 30 min. After 30 min. the reaction was concentrated in vacuo. The crude material was subjected to HPLC purification with a TFA modifier to yield mono(5-(1-(3,5-difluoro-4-((E)-4-(((S)-tetrahydrofuran-3-yl)ammonio)but-2-enamido)benzoyl)-1H-indol-4-yl)-4-methoxy-1,2-dimethyl-6-(trifluoromethyl)-1H-benzo[d]imidazol-3-ium) mono(2,2,2-trifluoroacetate). (Ex. 116) ¹H NMR (499 MHz, MeOD) δ 8.48 (d, J=8.4 Hz, 1H), 7.99 (s, 1H), 7.57 (d, J=7.6 Hz, 2H), 7.49 (t, J=7.9 Hz, 1H), 7.41 (d, J=3.8 Hz, 1H), 7.28 (d, J=7.2 Hz, 1H), 7.01-6.87 (m, 1H), 6.63 (d, J=15.4 Hz, 1H), 6.22 (d, J=3.7 Hz, 1H), 4.16-3.92 (m, 9H), 3.89-3.81 (m, 1H), 3.77 (q, J=8.3 Hz, 1H), 3.71 (s, 3H), 2.84 (s, 3H), 2.44 (m, 1H), 2.07 (m, 1H), [M+H]⁺ m/z: 668.

### Synthesis of (S,E)-4-(((tert-butoxycarbonyl)(tetrahydrofuran-3-yl)amino)but-2-enoic acid (Int-A)

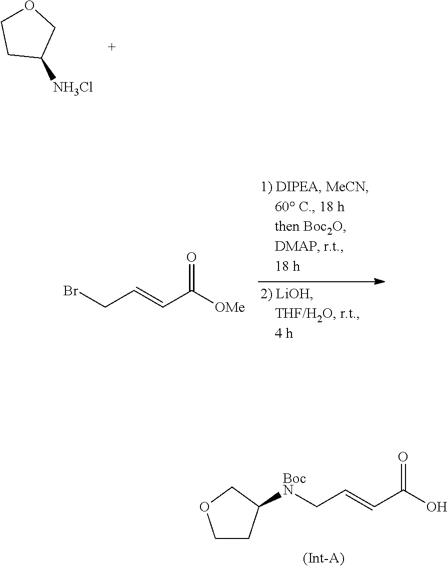

A 500 mL round bottom flask was charged with (S)-tetrahydrofuran-3-amine and HCl (5.32 g, 43.0 mmol). MeCN (196 ml) was added, and the reaction was fitted with a reflux condenser, septum, and exit needle. The reaction was heated to 60° C. to dissolve the salt. N,N-Diisopropylethyamine (7.38 ml, 42.2 mmol) was added via syringe. After approx. 1 h. the salt was dissolved. N,N-Diisopropylethyamine (7.38 ml, 42.2 mmol) was added via syringe, and methyl (E)-4-bromobut-2-enoate (4.61 ml. 39.1 mmol) was added slowly dropwise. The reaction stirred at 60° C. for 18 h. After 18 hours, the reaction was cooled to room temperature. The reflux condenser was removed, and 4-dimethylaminopyridine (0.478 g, 3.91 mmol) and di-tert-butyl dicarbonate (8.53 g, 39.1 mmol) was added. The reaction stirred at room temperature for 18 h. After 18 h, the reaction was concentrated in vacuo. The crude reaction mixture was dissolved in EtOAc (100 mL) and water (200 mL). The mixture was poured into a separatory funnel, shaken, and separated. The aqueous phase was washed 2× with EtOAc (100 mL each), and the combined organics were washed with brine (200 mL). The combined organics were dried over MgSO₄, filtered, and concentrated to provide a dark orange oil. The crude reaction mixture was purified via silica gel chromatography (0% to 25% EtoAc in hexanes) to yield methyl (R,E)-4-((tert-butoxycarbonyl)(tetrahydrofuran-3-yl)amino)but-2-enoate. [M+Na]⁺ m/z: 308.

Methyl (S,E)-4-((tert-butoxycarbonyl)(tetrahydrofuran-3-yl)amino)but-2-enoate (6.78 g. 23.76 mmol) was added to a 250 mL round bottom flask with stir bar. THF (47.5 ml) and water (47.5 ml) were added, followed by lithium hydroxide monohydrate (3.99 g. 95 mmol), added in one portion. The reaction was allowed to stir at room temperature for 4 hours. The reaction was diluted with EtOAc and water (50 mL each). 1 M Citric acid was added until the reaction mixture was pH=7. The reaction was transferred into a separatory funnel, and the layers were shaken and separated. The aqueous phase was washed with EtOAc (2×100 mL). The combined organics were washed with brine (200 mL), dried over MgSO₄, filtered and concentrated. (S,E)-4-((tert-butoxycarbonyl)(tetrahydrofuran-3-yl)amino)but-2-enoic acid (Int-A). [M+Na]⁺ m/z: 294.

### Example 117

### (E)-N-(2,6-difluoro-4-(8-(4-methoxy-1-methyl-6-(trifluoromethyl-1H-indazol-5-yl)indolizine-3-carbonyl)phenyl)-4-(((1r,4r)-4-methoxycyclohexyl)amino)but-2-enamide

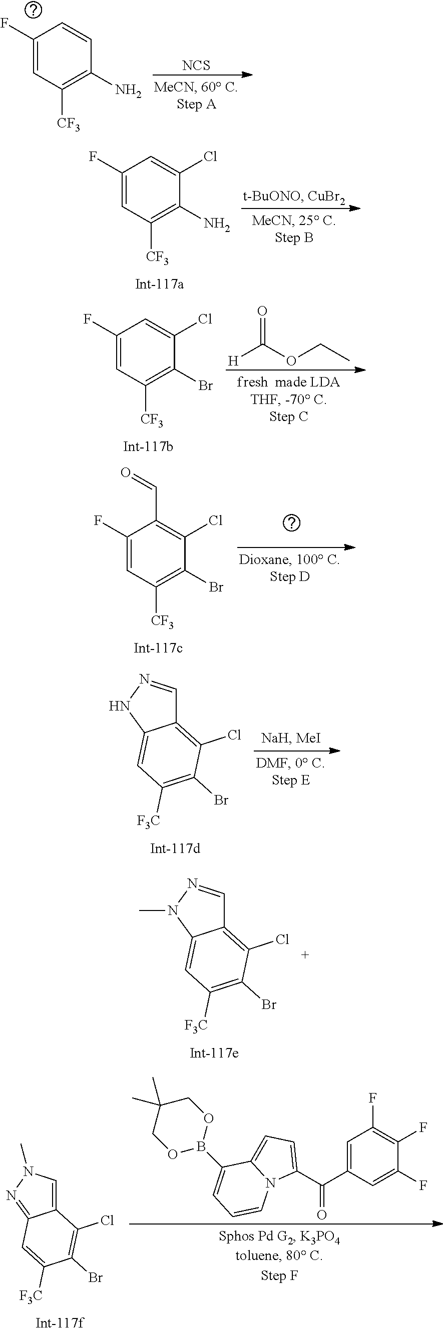

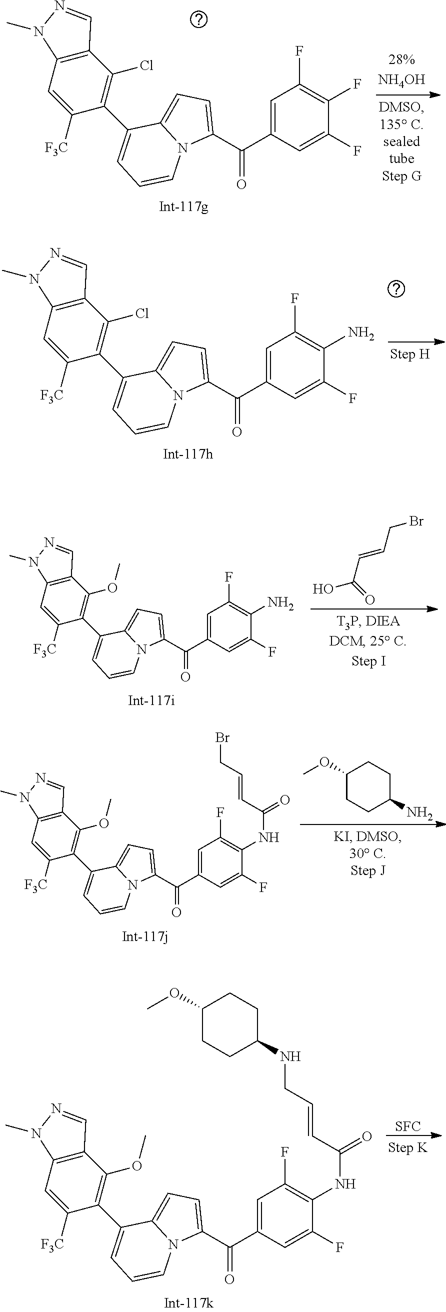

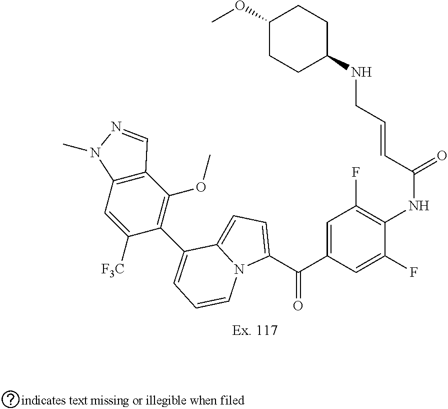 [SMILES: COc1c(-c2cccn3c(C(=O)c4cc(F)c(NC(=O)/C=C/CN[C@H]5CC[C@H](OC)CC5)c(F)c4)ccc23)c(C(F)(F)F)cc2c1cnn2C]

### Step A: 2-chloro-fluoro-6-(trifluoromethyl)aniline (Int-117a)

To a solution of 4-fluoro-2-(trifluoromethyl)aniline (40.0 g, 223 mmol) in MeCN (200 mL) was added 1-chloropyrrolidine-235-dione (32.8 g, 246 mol) at 25° C. Then this reaction solution was stirred at 60° C. for 6 h. The reaction mixture was quenched with H₂O (200 mL). The aqueous layer was extracted with EtOAc (200 mL×2). The combined organic layers were dried over Na₂SO₄ and filtered. The filtrate was concentrated under reduced pressure to give the residue which was purified by flash silica gel chromatography (ISCO; 120 g Agela Silica Flash Column. Eluent of 100% Pet. ether gradient a 30 mL/min) to give 2-chloro-4-fluoro-6-(trifluoromethyl)aniline (Int-117a). MS (ESI) [M+H]⁺: m/z 214.

### Step B: 2-bromo-1-chloro-5-fluoro-3-(trifluoromethyl)benzene (Int-117b)

To a solution of 2-chloro-4-fluoro-6-(trifluoromethyl)aniline (40.0 g, 187 mmol) and tert-butyl nitrite (30.9 g, 300 mmol) in MeCN (600 mL) was added copper(II) bromide (66.9 g. 300 mmol) at 0° C. under N₂ protection. Then this reaction solution was stirred at 25° C. for 5 h. The reaction mixture was quenched with H₂O (200 mL) and brine (200 mL) at 0° C. The reaction mixture was filtered, and the filtrate was extracted with EtOAc (600 mL×2). The combined organic layers were dried over Na₂SO₄ and filtered. The filtrate was concentrated under reduced pressure to give the residue which was purified by flash silica gel chromatography (ISCO; 120 g Agela Silica Flash Column, Eluent of 100% Pet. ether gradient @ 30 mL/min) to give 2-bromo-1-chloro-5-fluoro-3-(trifluoromethyl)benzene (Int-117b). ¹H NMR (400 MHz, CDCl₃) δ 7.45 (dd, J=2.7, 7.6 Hz, 1H), 7.39 (dd, J=2.7, 8.3 Hz, 1H).

### Step C: 3-bromo-2-chloro-6-fluoro-4-(trifluoromethyl)benzaldehyde (Int-117c)

To a solution of diisopropylamine (17.4 mL, 124 mmol) in THF (230 mL) was added n-butyllithium (43.1 mL, 108 mmol) (2.5 M in THF) at −70° C. under N₂ protection. The reaction mixture was stirred at −70° C. for 15 min. A solution of 2-bromo-1-chloro-5-fluoro-3-(trifluoromethyl)benzene (23.0 g, 83 mmol) in THF (50 mL) was added. The reaction mixture was stirred at −70° C. for 30 min. Then ethyl formate (10.2 mL, 124 mmol) was added. The reaction mixture was stirred at −70° C. for another 1 h. The reaction mixture was quenched with brine (200 mL) and saturated aqueous NH4Cl (200 mL) at 0° C. The aqueous layer was extracted with EtOAc (300 mL×2). The combined organic layers were dried over Na₂SO₄ and filtered. The filtrate was concentrated under reduced pressure to give the residue which was purified by flash silica gel chromatography (ISCO; 120 g Agela Silica Flash Column, Eluent of 10% EtOAc @ 30 mL/min) to give 3-bromo-2-chloro-6-fluoro-4-(trifluoromethyl)benzaldehyde (Int-117c). ¹H NMR (400 MHz, CDCl₃) δ 10.41 (s, 1H), 7.53 (d, J=10.0 Hz, 1H).

### Step D: 5-bromo-4-chloro-6-(trifluoromethyl)-1H-indazole (Int-117d)

To a solution of 3-bromo-2-chloro-6-fluoro-4-(trifluoromethyl)benzaldehyde (16.0 g. 52.4 mmol) in dioxane (25 mL) was added hydrazine (13.5 mL, 367 mmol) (85% in H₂O). Then reaction mixture was stirred at 100° C. for 16 h. The reaction mixture was concentrated to give the residue which was diluted with Pet. ether (30 mL) and EtOAc (10 mL), and yellow precipitate was formed. The solid was collected by filtration and dried in vacuum to give 5-bromo-4-chloro-6-(trifluoromethyl)-1H-indazole (Int-117d). MS (ESI) [M+H]⁺: m/z 298.8, 300.8

### Step E: 5-bromo-4-chloro-1-methyl-6-(trifluoromethyl)-1H-indazole (Int-117e) and 5-bromo-4-chloro-2-methyl-6-(trifluoromethyl)-2H-indazole (Int-117f)

To a solution of 5-bromo-4-chloro-6-(trifluoromethyl)-1H-indazole (10.3 g, 34.4 mmol) in DMF (150 mL) was added NaH (2.75 g, 68.8 mmol) (60% in mineral oil) at 0° C. under N₂ protection. The reaction mixture was stirred at 0° C. for 5 min. then Mel (2.6 mL, 41.3 mmol) was added. The reaction mixture was stirred at 0° C. for 1 h. The reaction mixture was quenched with brine (100 mL) and saturated aqueous NH₄Cl (100 mL) at 0° C. The aqueous layer was extracted with EtOAc (50 mL×2). The combined organic layers were dried over Na₂SO₄ and filtered. The filtrate was concentrated under reduced pressure to give the residue which was purified by flash silica gel chromatography (ISCO; 120 g Agela Silica Flash Column. Eluent of 0-15% EtOAc/Pet. ether @30 mL/min) to give 5-bromo-4-chloro-1-methyl-6-(trifluoromethyl)-1H-indazole (Int-117e) and 5-bromo-4-chloro-2-methyl-6-(trifluoromethyl)-2H-indazole (Int-117f). MS (ESI) [M+H]⁺: m/z 313.0, 315.0

### Step F: (8-(4-chloro-1-methyl-6-(trifluoromethyl)-1H-indazol-5-yl)indolizin-3-yl)(3,4,5-trifluorophenyl)methanone (Int-117g)

A mixture of 5-bromo-4-chloro-1-methyl-6-(trifluoromethyl)-1H-indazole (1.50 g, 4.78 mmol), (8-(5,5-dimethyl-1,3,2-dioxaborinan-2-yl)indolizin-3-yl)(3,4,5-trifluorophenyl)methanone (2.13 g, 5.50 mmol), chloro(2-dicyclohexylphosphino-2′,6′-dimethoxy-1,1′-biphenyl)[2-(2′-amino-1,1′-biphenyl)]palladium(II) (0.345 g, 0.478 mmol) and potassium phosphate tribasic (4.8 mL, 9.57 mmol) (2 M in H₂O) in toluene (30 mL) was degassed and backfilled with N₂ 3 times. The reaction mixture was stirred at 80° C. for 1.5 h. The reaction mixture was diluted with EtOAc (20 mL) and H₂O (20 mL). The mixture was filtered, and the aqueous layer was separated and extracted with EtOAc (20 mL×3). The combined organic layers were washed with brine (20 mL), dried over MgSO₄ and filtered. The filtrate was concentrated under reduced pressure to give the residue which was purified by flash silica gel chromatography (ISCO®; 20 g Agela® Silica Flash Column, Eluent of 0˜42% EtOAc/Pet. ether gradient @ 30 mL/min, dry loaded) to give (8-(4-chloro-1-methyl-6-(trifluoromethyl)-1H-indazol-5-yl)indolizin-3-yl)(3,4,5-trifluorophenyl)methanone (Int-117g). MS (ESI) [M+H]⁺: m/z 508.1

### Step G: (4-amino-3,5-difluorophenyl)(8-(4-chloro-1-methyl-6-(trifluoromethyl)-1H-indazol-5-yl)indolizin-3-yl)methanone (Int-117h)

To a solution of (8-(4-chloro-1-methyl-6-(trifluoromethyl)-1H-indazol-5-yl)indolizin-3-yl)(3,4,5-trifluorophenyl)methanone (200 mg, 0.394 mmol) in DMSO (2 mL) was added ammonium hydroxide solution (11.3 mL, 91 mmol) (28%). The reaction mixture was stirred at 135° C. for 24 h using a sealed tube. The reaction mixture was diluted with EtOAc (10 mL) and H₂O (5 mL). The aqueous layer was separated and extracted with EtOAc (10 mL×3). The combined organic layers were washed with brine (15 mL), dried over Na₂SO₄ and filtered. The filtrate was concentrated under reduced pressure to give the residue which was purified by Prep-TLC (SiO₂, Pet. ether/EtOAc=2:1, v/v) to give (4-amino-3,5-difluorophenyl)(8-(4-chloro-1-methyl-6-trifluoromethyl)-1H-indazol-5-yl)indolizin-3-yl)methanone (Int-117h). MS (ESI) [M+H]⁺: m/z 505.1

### Step H: (4-amino-3,5-difluorophenyl)(8-(4-methoxy-1-methyl-6-(trifluoromethyl)-1H-indazol-5-yl)indolizin-3-yl)methanone (Int-117i)

A mixture of (4-amino-3,5-difluorophenyl)(8-(4-chloro-1-methyl-6-(trifluoromethyl)-1H-indazol-5-yl)indolizin-3-yl)methanone (100 mg, 0.198 mmol), Cs₂CO₃ (129 mg, 0.396 mmol), MeOH (0.16 mL, 3.96 mmol) and (2-(Di-1-adamantylphosphino)morpholinobenzene)[2-(2′-amino-1,1′-biphenyl)]palladium(II) methanesulfonate (28.1 mg, 0.034 mmol) in dioxane (2.0 mL) was set up in a glove box. The reaction mixture was stirred at 80° C. for 12 h. The reaction mixture was diluted with EtOAc (10 mL) and filtered. The filtrate was concentrated under reduced pressure to give the residue which was purified by Prep-TLC (SiO₂, Pet. ether/EtOAc=5:1, v/v) to give (4-amino-3,5-difluorophenyl)(8-(4-methoxy-1-methyl-6-trifluoromethyl)-1H-indazol-5-yl)indolizin-3-yl)methanone (Int-117i). MS (ESI) [M+H]⁺: m/z 501.2

### Step I: (E)-4-bromo-N-(2,6-difluoro-4-(8-(4-methoxy-1-methyl-6-(trifluoromethyl)-1H-indazol-5-yl)indolizine-3-carbonyl)phenyl)but-2-enamide (Int-117j)

To a solution of (4-amino-3,5-difluorophenyl)(8-(4-methoxy-1-methyl-6-(trifluoromethyl)-1H-indazol-5-yl)indolizin-3-yl)methanone (40.0 mg, 0.080 mmol), (E)-4-bromobut-2-enoic acid (52.8 mg, 0.320 mmol) and DIEA (0.14 mL, 0.799 mmol) in DCM (1.5 mL) was added T3P solution (203 mg, 0.320 mmol) (50% in EtOAc). The reaction mixture was stirred at 25° C. for 1 h. The reaction mixture was diluted with DCM (2 mL) and NaHCO₃ (2 mL). The aqueous layer was separated and was extracted with DCM (2 mL×3). The combined organic layers were washed with brine (2 mL), dried over Na₂SO₄ and filtered. The filtrate was concentrated under reduced pressure to give (E)-4-bromo-N-(2,6-difluoro-4-(8-(4-methoxy-1-methyl-6-(trifluoromethyl)-1H-indazol-5-yl)indolizine-3-carbonyl)phenyl)but-2-enamide (Int-117j). MS (ESI) [M+H]⁺: m/z 647.0, 649.0

### Step J: (E)-N-(2,6-difluoro-4-(8-(4-methoxy-1-methyl-6-(trifluoromethyl)-1H-indazol-5-yl)indolizine-3-carbonyl)phenyl)-4-(((1r,4r)-4-methoxycyclohexyl)amino)but-2-enamide (Int-117k)

To a solution of (E)-4-bromo-N-(2,6-difluoro-4-(8-(4-methoxy-1-methyl-6-(trifluoromethyl)-1H-indazol-5-yl)indolizine-3-carbonyl)phenyl)but-2-enamide (51.7 mg, 0.080 mmol) and (1r,4r)-4-methoxycyclohexan-1-amine (41.3 mg, 0.319 mmol) in DMSO (0.5 mL) was added KI (26.5 mg, 0.160 mmol). The reaction mixture was stirred at 30° C. for 1 h. LC-MS showed all the starting material was consumed completely. The reaction mixture was filtered, and the filtrate was purified by Prep-HPLC (Column Welch Xtimate C18 150*25 mm*5 um. Condition water (TFA)-ACN Begin B 33. End B 53 Gradient Time (min) 11, 100% B Hold Time (min) 2 FlowRate (mL/min) 25. Injections 2) to give (E)-N-(2,6-difluoro-4-(8-(4-methoxy-1-methyl-6-(trifluoromethyl)-1H-indazol-5-yl)indolizine-3-carbonyl)phenyl)-4-(((1r,4r)-4-methoxycyclohexyl)amino)but-2-enamide (Int-117k). MS (ESI) [M+H]⁺: m/z 696.2

### Step K: (E)-N-(2,6-difluoro-4-(8-(4-methoxy-1-methyl-6-(trifluoromethyl)-1H-indazol-5-yl)indolizine-3-carbonyl)phenyl)-4-(((1r,4r)-4-methoxycyclohexyl)amino)but-2-enamide (Ex. 117)

Racemic (E)-N-(2,6-difluoro-4-(8-(4-methoxy-1-methyl-6-(trifluoromethyl)-1H-indazol-5-yl)indolizine-3-carbonyl)phenyl)-4-(((1r,4r)-4-methoxycyclohexyl)amino)but-2-enamide (19 mg, 0.027 mmol) was separated by preparative SFC (Column DAICEL CHIRALPAK AD (250 mm*30 mm. 10 um). Condition 0.1% NH₃H₂O EtOH Begin B 50%. End B 50% Gradient Time (min), 100% B Hold Time (min) FlowRate (mL/min) 80, Injections 45) to afford (E)-N-(2,6-difluoro-4-(8-(4-methoxy-1-methyl-6-(trifluoromethyl)-1H-indazol-5-yl)indolizine-3-carbonyl)phenyl)-4-(((1r,4r)-4-methoxycyclohexyl)amino)but-2-enamide (Ex. 117, the second eluting isomer from SFC). MS (ESI) [M+H]⁺: m/z 696.2 ¹H NMR (400 MHz, MeOD) δ 9.94 (d, J=6.85 Hz, 1H), 8.43 (s, 1H), 7.78 (s, 1H), 7.49 (d, J=8.07 Hz, 2H), 7.37 (d, J=4.89 Hz, 1H), 7.21-7.25 (m, 1H), 7.15-7.20 (m, 1H), 7.00 (td, J=5.93, 15.53 Hz, 1H), 6.37 (d, J=15.41 Hz, 1H), 6.09 (d, J=4.65 Hz, 1H), 4.18 (s, 3H), 4.06 (s, 3H), 3.55 (d, J=5.38 Hz, 2H), 3.34-3.36 (m, 3H). 3.13-3.26 (m, 1H), 2.56-2.69 (m, 1H), 2.02-2.13 (m, 4H), 1.20-1.26 (m, 4H).

### Example 118

### (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide

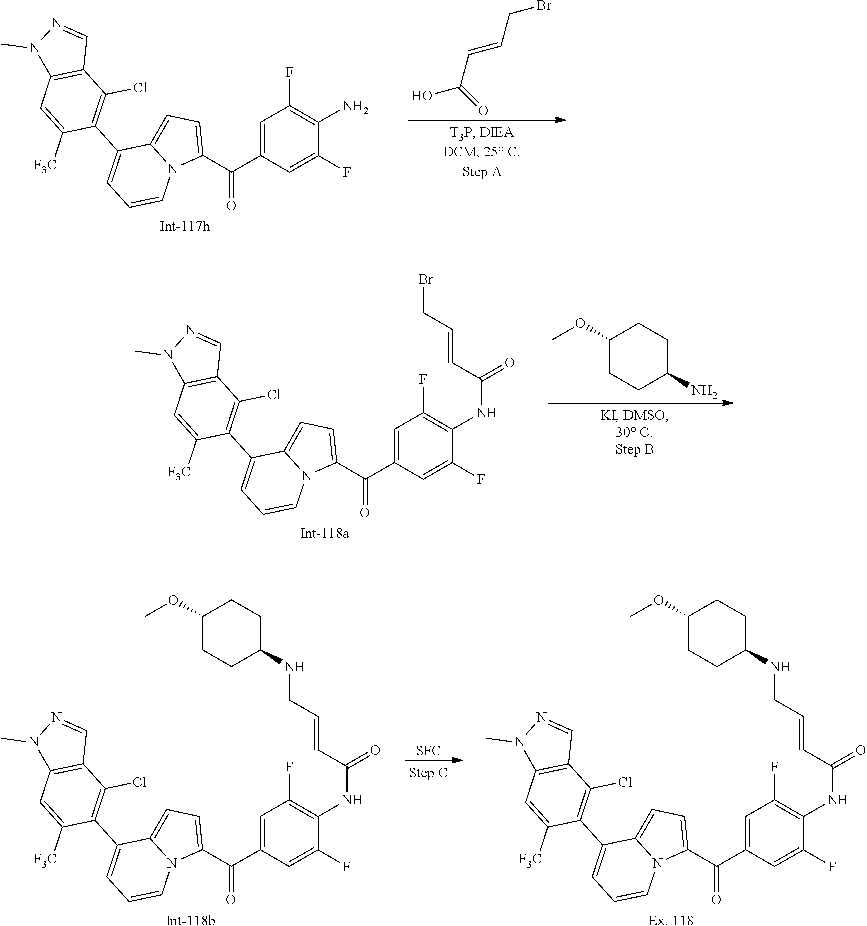

### Step A: (E)-4-bromo-N-(4-(8-(4-chloro-1-methyl-6-(trifluoromethyl)-1H-indazol-5-yl)indolizine-3-carbonyl)-2,6-difluorophenyl)but-2-enamide (Int-118a)

To a solution of (4-amino-3,5-difluorophenyl)(8-(4-chloro-1-methyl-6-(trifluoromethyl)-1H-indazol-5-yl)indolizin-3-yl)methanone (Int-117h)(70.0 mg, 0.139 mmol). (E)-4-bromobut-2-enoic acid (68.6 mg, 0.416 mmol) and DIEA (0.24 mL, 1.39 mmol) in DCM (1.5 mL) was added T3P solution (353 mg, 0.555 mmol) (50% in EtOAc). The reaction mixture was stirred at 25° C. for 1 h. The reaction mixture was diluted with DCM (2 mL) and NaHCO₃ (2 mL). The aqueous layer was separated and extracted with DCM (2 mL×3). The combined organic layers were washed with brine (2 mL), dried over Na₂SO₄ and filtered. The filtrate was concentrated under reduced pressure to give (E)-4-bromo-N-(4-(8-(4-chloro-1-methyl-6-(trifluoromethyl)-1H-indazol-5-yl)indolizine-3-carbonyl)-2,6-difluorophenyl)but-2-enamide (Int-118a). MS (ESI) [M+H]⁺: m/z 651.0, 653.0

### Step B: (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide (Int-118b)

To a solution of (E)-4-bromo-N-(4-(8-(4-chloro-1-methyl-6-(trifluoromethyl)-1H-indazol-5-yl)indolizine-3-carbonyl)-2,6-difluorophenyl)but-2-enamide (86.0 mg, 0.132 mmol) and (1r,4r)-4-methoxycyclohexan-1-amine (85.0 mg, 0.660 mmol) in DMSO (0.5 mL) was added KI (43.8 mg, 0.264 mmol). The reaction mixture was stirred at 30° C. for 1 h. The reaction mixture was filtered and was purified by Prep-HPLC (Column Welch Xtimate C18 150*25 mm*5 um, Condition water (TFA)-ACN Begin B 35. End B 55 Gradient Time (min) 11, 100% B Hold Time (min) 2 FlowRate (mL/min) 25. Injections 7) to give (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide (Int-118b). MS (ESI) [M+H]⁺: m/z 700.2

### Step C: (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide (Ex. 118)

Racemic (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide (21 mg, 0.030 mmol) was separated by preparative SFC (Column DAICEL CHIRALPAK AD-H (250 mm*30 mm. 5 um), Condition 0.1% NH₃H₂O EtOH Begin B 50%, End B 50% Gradient Time (min), 100% B Hold Time (min). FlowRate (mL/min) 80, Injections 60) to afford (E)-N-(4-(8-(4-chloro-1-methyl-6-(trifluoromethyl)-1H-indazol-5-yl)indolizine-3-carbonyl)-2,6-difluorophenyl)-4-(((1r,4r)-4-methoxycyclohexyl)amino)but-2-enamide (Ex. 118, the second eluting isomer from SFC). MS (ESI) [M+H]⁺: m/z 700.2 ¹H NMR (400 MHz, MeOD) δ 10.00 (d, J=7.09 Hz, 1H), 8.23 (d, J=6.11 Hz, 2H), 7.49 (d, J=7.83 Hz, 2H), 7.42 (d, J=4.89 Hz, 1H), 7.26-7.30 (m, 1H), 7.19-7.25 (m, 1H), 7.01 (td, J=5.96, 15.47 Hz, 1H), 6.34 (d, J=15.41 Hz, 1H), 6.06 (d, J=4.65 Hz, 1H), 4.24 (s, 3H), 3.49 (d, J=5.14 Hz, 2H), 3.33-3.35 (m, 3H), 3.13-3.24 (m, 1H), 2.54 (dd, J=2.81, 6.97 Hz, 1H), 2.00-2.11 (m, 4H), 1.17-1.25 (m, 4H).

### Example 119

### (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide

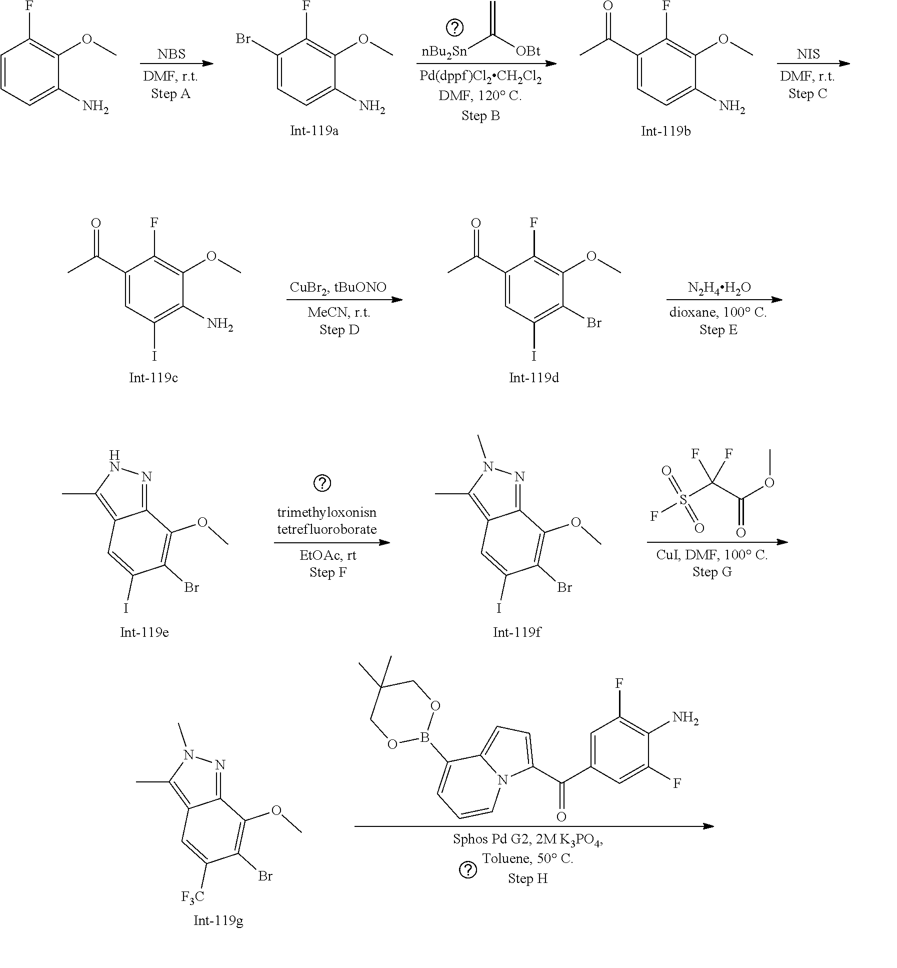

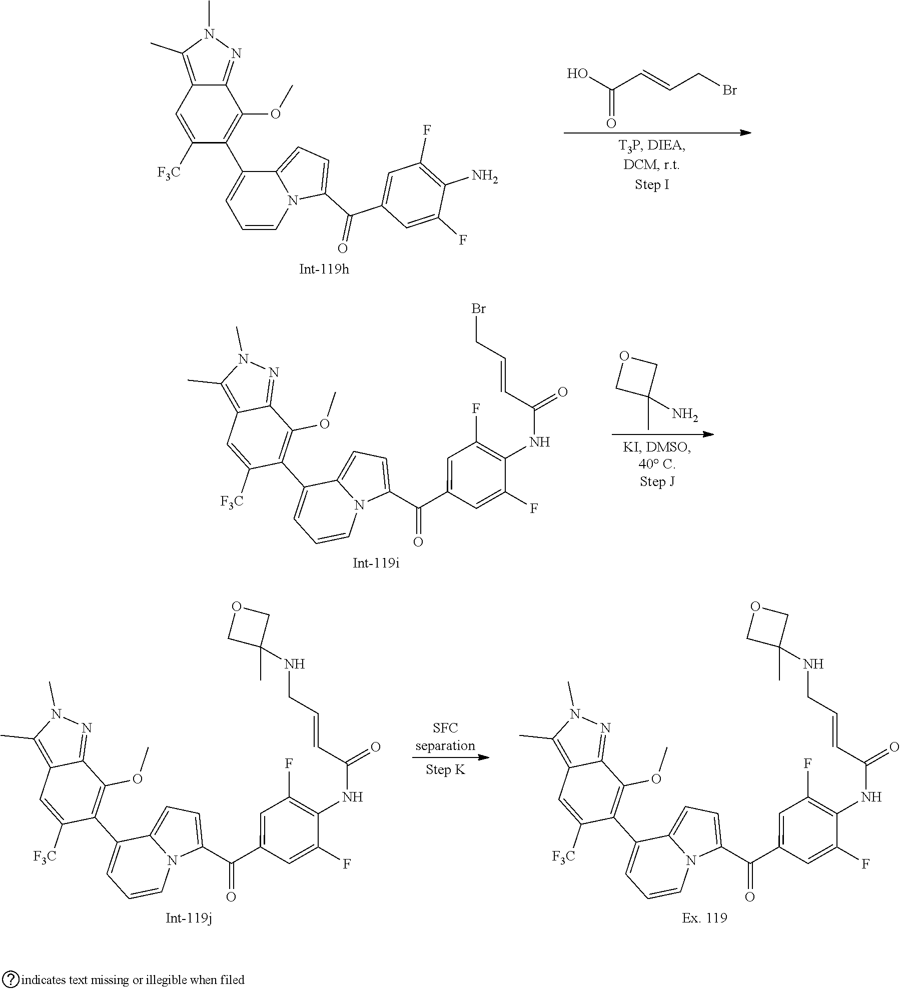

### Step A: 4-bromo-3-fluoro-2-methoxyaniline (Int-119a)

To a stirred solution of 3-fluoro-2-methoxyaniline (2 g, 14.17 mmol) in DMF (20 mL) was added NBS (2.396 g, 13.46 mmol) at 20° C. After addition, the reaction was stirred at 20° C. for 16 h. Water (300 mL) was added to the reaction, and then the reaction was extracted with EtOAc (50 mL*2). The combined organic layer was washed with brine, dried over Na₂SO₄, filtered and concentrated under reduced pressure to give crude. The crude product was purified by flash silica gel chromatography (ISCO®; Agela® Flash Column Silica-CS (12 g) Eluent of 0˜20% Ethyl acetate/Petroleum ether gradient @ 40 mL/min) to give 4-bromo-3-fluoro-2-methoxyaniline (Int-119a). ¹H NMR (400 MHz, MeOD) δ 6.95 (dd, J=7.21, 8.68 Hz, 1H), 6.46 (dd, J=1.59, 8.68 Hz, 1H), 3.82 (d, J=0.73 Hz, 3H).

### Step B: 1-(4-amino-2-fluoro-3-methoxyphenyl)ethan-1-one (Int-119b)

To a stirred solution of 4-bromo-3-fluoro-2-methoxyaniline (2 g, 9.09 mmol) in DMF (30 mL) was added [1,1′-bis(diphenylphosphinoferrocene]dichloropalladium (II) (0.665 g. 0.909 mmol), tributyl(1-ethoxyvinyl)stannane (3.99 ml. 11.82 mmol) at 20° C.; after the addition, the reaction was stirred at 120° C. for 16 h. After cooling to room temperature, 1N aqueous hydrochloric acid (5 mL) was added to the reaction mixture, and the mixture was stirred at room temperature for 1 h. The reaction mixture was quenched with water (30 mL), extracted with ethyl acetate (3*30 mL). The combined organic layer was washed with saturated brine, dried over anhydrous magnesium sulfate, and concentrated under reduced pressure. The crude product was purified by flash silica gel chromatography (ISCO®; Agela® Flash Column Silica-CS (12 g) Eluent of 0˜30% Ethyl acetate/Petroleum ether gradient @ 40 mL/min) to give 1-(4-amino-2-fluoro-3-methoxyphenyl)ethan-1-one (Int-119b). ¹H NMR (400 MHz, CDCl₃) δ 7.49 (q, J=8.15 Hz, 1H), 6.47 (br t, J=8.44 Hz, 1H), 3.90 (br d, J=8.31 Hz, 3H), 2.54 (s, 3H).

### Step C: 1-(4-amino-2-fluoro-5-iodo-3-methoxyphenyl)ethan-1-one (Int-119c)

To a stirred solution of 1-(4-amino-2-fluoro-3-methoxyphenyl)ethan-1-one (4.5 g, 24.57 mmol) in DMF (50 mL) was added 1-iodopyrrolidine-2,5-dione (5.25 g, 23.34 mmol) in DMF (20 mL) dropwise at 20° C.; after the addition was finished, the reaction was stirred at 20° C. for 16 h. The reaction was quenched with water (20 mL), extracted with EtOAc (100 mL*2). The combined organic layer was washed with brine, dried over Na₂SO₄, filtered and concentrated under reduced pressure to give crude. The crude product was purified by flash silica gel chromatography (ISCO®; Agela® Flash Column Silica-CS (20 g) Eluent of 0-70% Ethyl acetate/Petroleum ether gradient @ 80 mL/min) to give 1-(4-amino-2-fluoro-5-iodo-3-methoxyphenyl)ethan-1-one (Int-119c). ¹H NMR (400 MHz, MeOD) δ 7.90 (d, J=7.34 Hz, 1H), 3.86 (s, 3H), 2.44-2.52 (m, 3H).

### Step D: 1-(4-bromo-2-fluoro-5-iodo-3-methoxyphenyl)ethan-1-one (Int-119d)

To a stirred solution of 1-(4-amino-2-fluoro-5-iodo-3-methoxyphenyl)ethan-1-one (1 g, 3.24 mmol) in MeCN (10 mL) was added copper(II) bromide (1.445 g, 6.47 mmol) and tert-butyl nitrite (0.500 g, 4.85 mmol) at 20° C.; after the addition was finished, the reaction was stirred at 20° C. for 16 h. The crude product was purified by HPLC (Pe. ether/ethyl acetate 5:1 as eluent) to give 1-(4-bromo-2-fluoro-5-iodo-3-methoxyphenyl)ethan-1-one (Int-119d). ¹H NMR (400 MHz, CDCl₃) δ 8.08 (d, J=7.09 Hz, 1H), 3.97 (s, 3H), 2.64 (s, 3H).

### Step E: 6-bromo-5-iodo-7-methoxy-3-methyl-2H-indazole (Int-119e)

To a solution of 1-(4-bromo-2-fluoro-5-iodo-3-methoxyphenyl)ethan-1-one (8 g. 21.45 mmol) in dioxane (80 mL) was added hydrazinium hydroxide (4.42 g. 75 mmol. 85%). Then the mixture was stirred at 100° C. for 16 h. Then the reaction mixture was concentrated, and the residue was purified by flash silica gel chromatography (ISCO®: 24 g SepaFlash® Silica Flash Column. Eluent of 25% ethyl acetate in petroleum ether gradient @30 mL/min) to give 6-bromo-5-iodo-7-methoxy-3-methyl-2H-indazole (Int-119e). MS (ESI) [M+H]⁺: m/z 366.9, 368.9

### Step F: 6-bromo-5-iodo-7-methoxy-2,3-dimethyl-2H-indazole (Int-119f)

To a solution of 6-bromo-5-iodo-7-methoxy-3-methyl-2H-indazole (6 g, 16.35 mmol) in EtOAc (100 mL) was added trimethyloxonium tetrafluoroborate (7.25 g, 49.0 mmol). Then the mixture was stirred at 25° C. for 3 h. Then the reaction was quenched with water (100 mL), basified with aqueous NaHCO₃ to pH about 8 and extracted with EtOAc (100 mL*3). The combined organic layers were dried over Na₂SO₄, filtered and concentrated. The residue was purified by flash silica gel chromatography (ISCO®; 24 g SepaFlash® Silica Flash Column, Eluent of 30% ethyl acetate in petroleum ether gradient @ 30 mL/min) to give 6-bromo-5-iodo-7-methoxy-2,3-dimethyl-2H-indazole (Int-119f). MS (ESI) [M+H]⁺: m/z 380.8, 382.8

### Step G: 6-bromo-7-methoxy-2,3-dimethyl-5-(trifluoromethyl)-2H-indazole (Int-119g)

To a solution of 6-bromo-5-iodo-7-methoxy-2,3-dimethyl-2H-indazole (3.8 g, 9.97 mmol) in DMF (40 mL) was added copper (I) iodide (5.70 g, 29.9 mmol) and methyl 2,2-difluoro-2-(fluorosulfonyl)acetate (11.50 g, 59.8 mmol). The mixture was stirred at 85° C. for 2 h. The reaction was cooled to room temperature, quenched with water (30 mL), extracted with EtOAc (80 mL*3). The combined organic layer was washed with brine, dried over Na₂SO₄, filtered and concentrated under reduced pressure to give crude. The crude product was purified by flash silica gel chromatography (ISCO®; Agela® Flash Column Silica-CS (40 g) Eluent of 0˜30% Ethyl acetate/Petroleum ether gradient @ 40 mL/min) to give 6-bromo-7-methoxy-2,3-dimethyl-5-(trifluoromethyl)-2H-indazole (Int-119g). MS (ESI) [M+H]J: m/z 323.1, 325.1

### Step H: (4-amino-3,5-difluorophenyl)(8-(7-methoxy-2,3-dimethyl-5-(trifluoromethyl)-2H-indazol-6-yl)indolizin-3-yl)methanone (Int-119h)

To a solution of 6-bromo-7-methoxy-2,3-dimethyl-5-(trifluoromethyl)-2H-indazole (5(0) mg, 1.547 mmol) and (4-amino-3,5-difluorophenyl)(8-(5,5-dimethyl-1,3,2-dioxaborinan-2-yl)indolizin-3-yl)methanone (773 mg, 2.012 mmol) in toluene (10 mL) was added 2M aqueous K₃PO₄ (2.321 mL, 4.64 mmol) and chloro(2-dicyclohexylphosphino-2′,6′-dimethoxy-1,1′-biphenyl)[2-(2′-amino-1,1′-biphenyl)]palladium(II) (55.8 mg, 0.077 mmol) at 25° C. under N₂ atmosphere. The mixture was stirred at 80° C. for 2 h. The mixture was cooled, diluted with water (10 mL), extracted with EtOAc (3×10 mL), dried over Na₂SO₄, filtered and the solvent was evaporated under reduced pressure to give the crude product. The crude product was purified by flash silica gel chromatography (ISCO®; 20 g SepaFlash®, Silica Flash Column, Eluent of 0˜30% EtOAc/Pet. ether gradient @ 40 mL/min) to give (4-amino-3,5-difluorophenyl)(8-(7-methoxy-2,3-dimethyl-5-(trifluoromethyl)-2H-indazol-6-yl)indolizin-3-yl)methanone (Int-119h). MS (ESI) [M+H]⁺: m/z 515.1

### Step I: (E)-4-bromo-N-(2,6-difluoro-4-(8-(7-methoxy-2,3-dimethyl-5-(trifluoromethyl)-2H-indazol-6-yl)indolizine-3-carbonyl)phenyl)but-2-enamide (Int-119i)

To a solution of (4-amino-3,5-difluorophenyl)(8-(7-methoxy-2,3-dimethyl-5-(trifluoromethyl)-2H-indazol-6-yl)indolizin-3-yl)methanone (200 mg, 0.389 mmol) and (E)-4-bromobut-2-enoic acid (128 mg, 0.778 mmol) in DCM (4 mL) was added T3P (990 mg, 1.555 mmol) and DIEA (0.407 mL, 2.333 mmol) at 25° C. The mixture was stirred at 25° C. for 1 h. The mixture was diluted with water (3 mL), extracted with DCM (3×3 mL), dried over Na₂SO₄, and filtered, and the solvent was evaporated under reduced pressure to give the crude product. The crude product was purified by flash silica gel chromatography (ISCO®; 4 g SepaFlash® Silica Flash Column, Eluent of 0˜30% EtOAc/Pet. ether gradient @ 40 mL/min) to give (E)-4-bromo-N-(2,6-difluoro-4-(8-(7-methoxy-2,3-dimethyl-5-(trifluoromethyl)-2H-indazol-6-yl)indolizine-3-carbonyl)phenyl)but-2-enamide (Int-119i). MS (ESI) [M+H]⁺: m/z 661.0, 663.0

### Step J: (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide (Int-119j)

To a solution of (E)-4-bromo-N-(2,6-difluoro-4-(8-(7-methoxy-2,3-dimethyl-5-(trifluoromethyl)-2H-indazol-6-yl)indolizine-3-carbonyl)phenyl)but-2-enamide (90 mg. 0.136 mmol). 3-methyloxetan-3-amine, and HCl (84 mg, 0.680 mmol) in DMSO (1.5 mL) was added DIEA (0.119 mL, 0.680 mmol) and KI (45.2 mg, 0.272 mmol) at 25° C. under N₂ atmosphere. The mixture was stirred at 40° C. for 1 h. The mixture was cooled, diluted with water (2 mL), extracted with DCM (3×2 mL), dried over Na₂SO₄, and filtered, and the solvent was evaporated under reduced pressure to give the crude product. The crude product was purified by flash silica gel chromatography (ISCO®; 4 g SepaFlash® Silica Flash Column. Eluent of 0˜80% EtOAc/Pet. ether gradient @ 40 mL/min) to give (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide (Int-119j). MS (ESI) [M+H]⁺: m/z 668.2

### Step K: (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide (Ex. 119)

Racemic (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide (100 mg, 0.099 mmol) was separated by preparative SFC (Column: DAICEL CHIRALPAK AD (250 mm*30 mm, 10 um), Condition: 0.1% NH₃H₂O/EtOH. Mobile phase: A: CO₂ B: EtOH (0.1% NH₃H₂O), Gradient: from 40% of B, Flow rate: 80 mL/min, Column temp: 40° C.) to give (E)-N-(2,6-difluoro-4-(8-(7-methoxy-2,3-dimethyl-5-(trifluoromethyl)-2H-indazol-6-yl)indolizine-3-carbonyl)phenyl)-4-((3-methyloxetan-3-yl)amino)but-2-enamide (Ex. 119, the first eluting isomer from SFC). MS (ESI) [M+H]⁺: m/z 668.2 ¹H NMR (500 MHz, CD3OD) δ 9.97 (d, J=7.0 Hz, 1H), 7.97 (s, 1H), 7.52 (d, J=7.9 Hz, 2H), 7.40 (d, J=4.9 Hz, 1H), 7.33-7.18 (m, 2H), 7.11-6.90 (m, 1H), 6.66 (br d, J=15.4 Hz, 1H), 6.17 (d, J=4.7 Hz, 1H), 4.81 (br d, J=7.6 Hz, 2H). 4.60 (d, J=7.8 Hz, 2H), 4.25-4.15 (n, 3H), 4.03-3.95 (m, 5H), 2.76 (s, 3H), 1.85-1.68 (m, 3H).

### Example 120

### mono(7-(3-(3,5-difluoro-4-((E)-4-(((1r,4r)-4-methoxycyclohexyl)ammonio)but-2-enamido)benzoyl)indolizin-8-yl)-6-(trifluoromethyl)quinolin-1-ium) mono(2,2,2-trifluoroacetate)

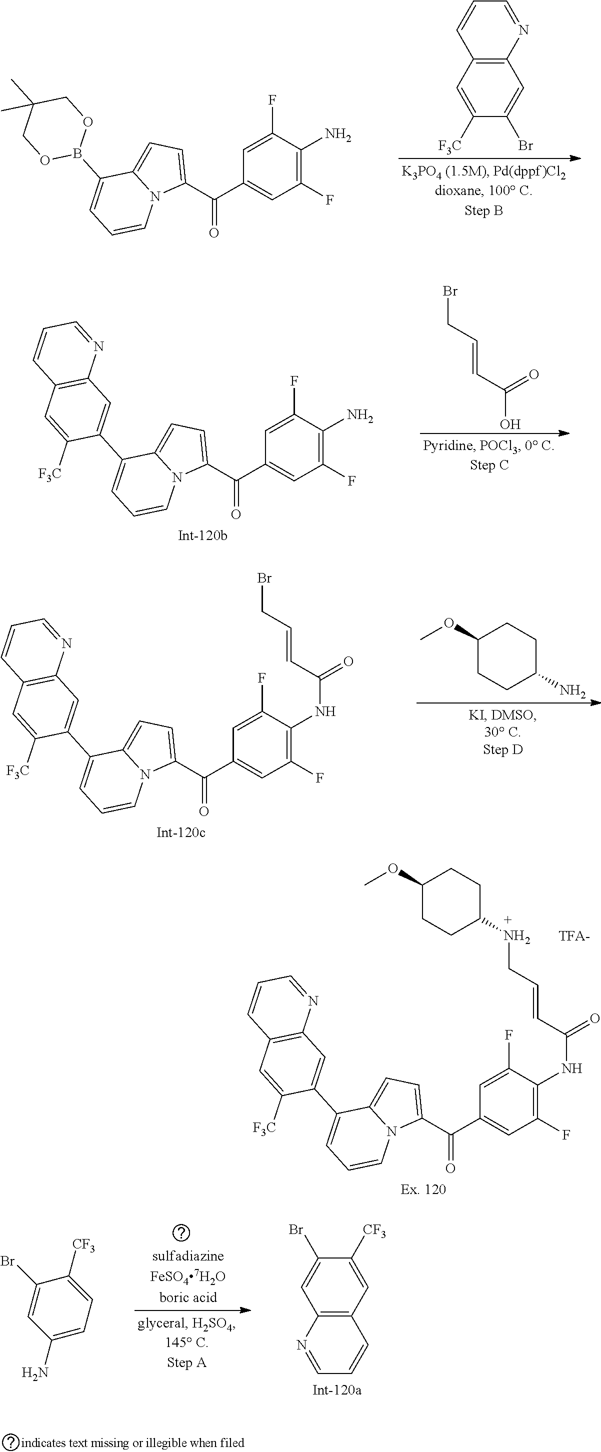

### Step A: 7-bromo-6-(trifluoromethyl)quinoline (Int-120a)

To a stirred solution of 3-bromo-4-(trifluoromethyl)aniline (160 mg, 0.667 mmol) in propane-1,2,3-triol (5 mL, 68.5 mmol) were added sulfadiazine (250 mg, 1.000 mmol), and iron(II) sulfate heptahydrate (37.1 mg, 0.133 mmol), followed by boric acid (63.9 mg, 1.067 mmol) at 25° C. The reaction mixture was cooled to 0° C. and conc. H₂SO₄ (0.5 mL) was added to the reaction mixture. The resulting mixture was heated at 145° C. for 3 h. The reaction was quenched with cold water (10 mL) and neutralized with NaHCO₃ solid until pH 8. The aqueous layer was extracted with CH₂Cl₂ (2×20 mL), and the combined organic phases were dried over anhydrous Na₂SO₄, filtered and concentrated in vacuo. The crude product was purified by flash silica gel chromatography (ISCO®; 4 g SepaFlash® Silica Flash Column. Eluent of 7% ethyl acetate in petroleum ether gradient @ 25 mU/min) to give 7-bromo-6-(trifluoromethyl)quinoline (Int-120a). MS (ESI) [M+H]⁺: m/z 276.0, 278.0

### Step B: (4-amino-3,5-difluorophenyl)(8-(6-(trifluoromethyl)quinolin-7-yl)indolizin-3-yl)methanone (Int-120b)

To a solution of (4-amino-3,5-difluorophenyl)(8-(5,5-dimethyl-1,3,2-dioxaborinan-2-yl)indolizin-3-yl)methanone (100 mg, 0.260 mmol) in 1,4-dioxane (1 mL) was added 7-brom-6-(trifluoromethyl)quinoline (71.9 ng, 0.260 mmol), [1,1′-bis(diphenylphosphino)ferrocene)dichloropalladium (II) (19.05 mg, 0.026 mmol) and potassium phosphate tribasic (0.069 mL, 0.104 mmol. 1.5M in H₂O) at 25° C. under N₂ atmosphere. The mixture was stirred at 10) ° C. for 2 h. The mixture was cooled, diluted with H₂O (2 mL), extracted with EtOAc (5×3 mL), dried over Na₂SO₄, and filtered, and the solvent was evaporated under reduced pressure to give the crude product. The crude product was purified by preparative TLC (SiO₂, petroleum ether:ethyl acetate=2:1) to give (4-amino-3,5-difluorophenyl)(8-(6-(trifluoromethyl)quinolin-7-yl)indolizin-3-yl)methanone (Int-120b). MS (ESI) [M+H+MeCN]⁺: m/z 509.1

### Step C: (E)-4-bromo-N-(2,6-difluoro-4-(8-(6-(trifluoromethyl)quinolin-7-yl)indolizine-3-carbonyl)phenyl)but-2-enamide (Int-120c)

To a solution of (E)-4-bromobut-2-enoic acid (40 mg, 0.242 mmol) in pyridine (0.4 mL) was added POCl₃ (0.04 mL, 0.429 mmol) at 0° C., and the reaction solution was stirred at 0° C. for 10 min. Then (4-amino-3,5-difluorophenyl)(8-(6-(trifluoromethyl)quinolin-7-yl)indolizin-3-yl)methanone (46 mg, 0.098 mmol, dissolved in pyridine (0.2 mL) was added to the reaction solution at 0° C., and the mixture was stirred at 0° C. for 30 min. The mixture was quenched with water (1 mL), extracted with EtOAc (3×2 mL), dried over Na₂SO₄, and filtered, and the solvent was evaporated under reduced pressure to give the crude product. The crude product was purified by preparative TLC (SiO₂, petroleum ether:ethyl acetate=2:1) to give (E)-4-bromo-N-(2,6-difluoro-4-(8-(6-(trifluoromethyl)quinolin-7-yl)indolizine-3-carbonyl)phenyl)but-2-enamide (Int-120c). MS (ESI) [M+H]⁺: m/z 614.0, 616.0

### Step D: mono(7-(3-(3,5-difluoro-4-((E)-4-(((1r,4r)-4-methoxycyclohexyl)ammonio)but-2-enamido)benzoyl)indolizin-8-yl)-6-(trifluoromethyl)quinolin-1-ium) mono(2,2,2-trifluoroacetate) (Ex. 120)

To a solution of (E)-4-bromo-N-(2,6-difluoro-4-(8-(6-(trifluoromethyl)quinolin-7-yl)indolizine-3-carbonyl)phenyl)but-2-enamide (10 mg, 0.016 mmol) in DMSO (0.1 mL) was added (1r,4r)-4-methoxycyclohexan-1-amine (10.52 mg, 0.081 mmol) and KI (8.11 mg, 0.049 mmol) at 25° C. The mixture was stirred at 30° C. for 2 h. The mixture was cooled, and the solvent was purified directly by reverse preparative HPLC (Column: Waters Xbridge BEH C18 100*25 mm*5 um; Condition: water (TFA)-ACN; Begin B-End B: 15-35; Gradient Time (min): 12; 100% B Hold Time (min): 2: FlowRate (mL/min): 25) to give mono(7-(3-(3,5-difluoro-4-((E)-4-(((1r,4r)-4-methoxycyclohexyl)ammonio)but-2-enamido)benzoyl)indolizin-8-yl)-6-(trifluoromethyl)quinolin-1-ium) mono(2,2,2-trifluoroacetate) (Ex. 120). MS (ESI) [M+H]⁺: m/z 663.3 ¹H NMR (400 MHz, MeOD) δ 10.02 (d, J=7.0 Hz, 1H), 9.11 (d, J=3.0 Hz, 1H), 8.61-8.79 (m, 2H), 8.17 (s, 1H), 7.79 (dd, J=8.4, 4.4 Hz, 1H), 7.55 (d, J=8.0 Hz, 2H), 7.46 (d, J=4.9 Hz, 1H), 7.39 (d, J=6.9 Hz, 1H), 7.23-7.33 (m, 1H), 6.86-7.03 (m, 1H), 6.61 (d, J=15.4 Hz, 1H), 6.22 (d, J=4.8 Hz, 1H), 3.88-4.00 (in, 1H), 3.96 (d, J=6.6 Hz, 1H), 3.38 (s, 3H), 3.12-3.29 (m, 2H), 2.22 (br d, J=9.1 Hz, 2H), 2.12-2.32 (m, 1H), 2.12-2.34 (m, 1H), 1.42-1.54 (m, 1H), 1.39-1.57 (m, 1H), 1.26-1.37 (m, 2H).

### Example 121

### (1r,4r)-N-((E)-4-((2,6-difluoro-4-(8-(2-methyl-1-oxo-6-(trifluoromethyl)-1,2,3,4-tetrahydroisoquinolin-7-yl)indolizine-3-carbonyl)phenyl)amino)-4-oxobut-2-en-1-yl)-4-methoxycyclohexan-1-aminium 2,2,2-trifluoroacetate

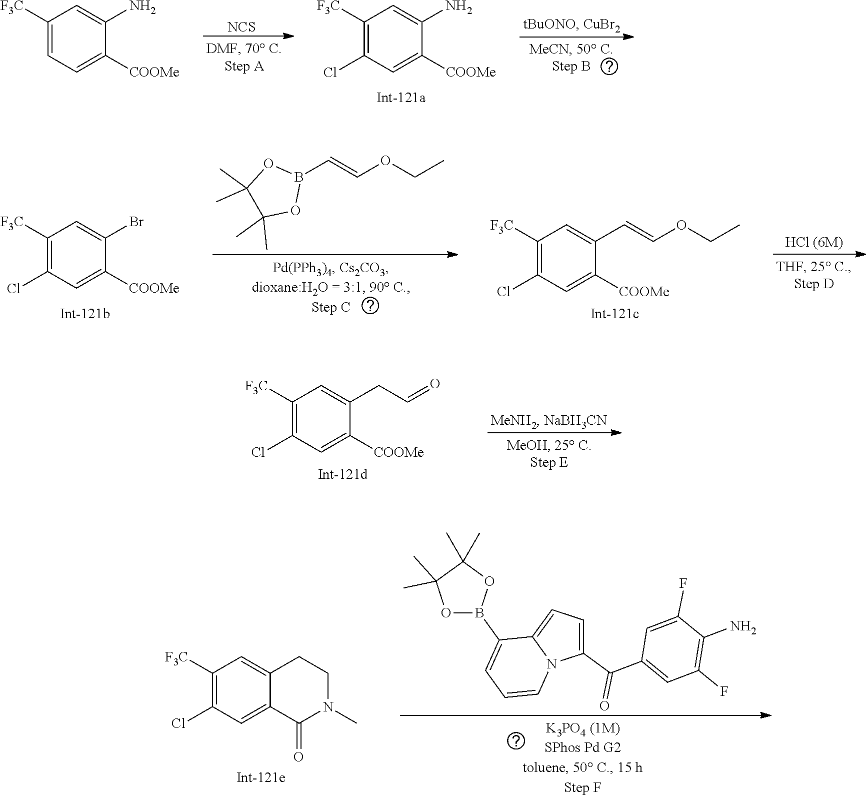

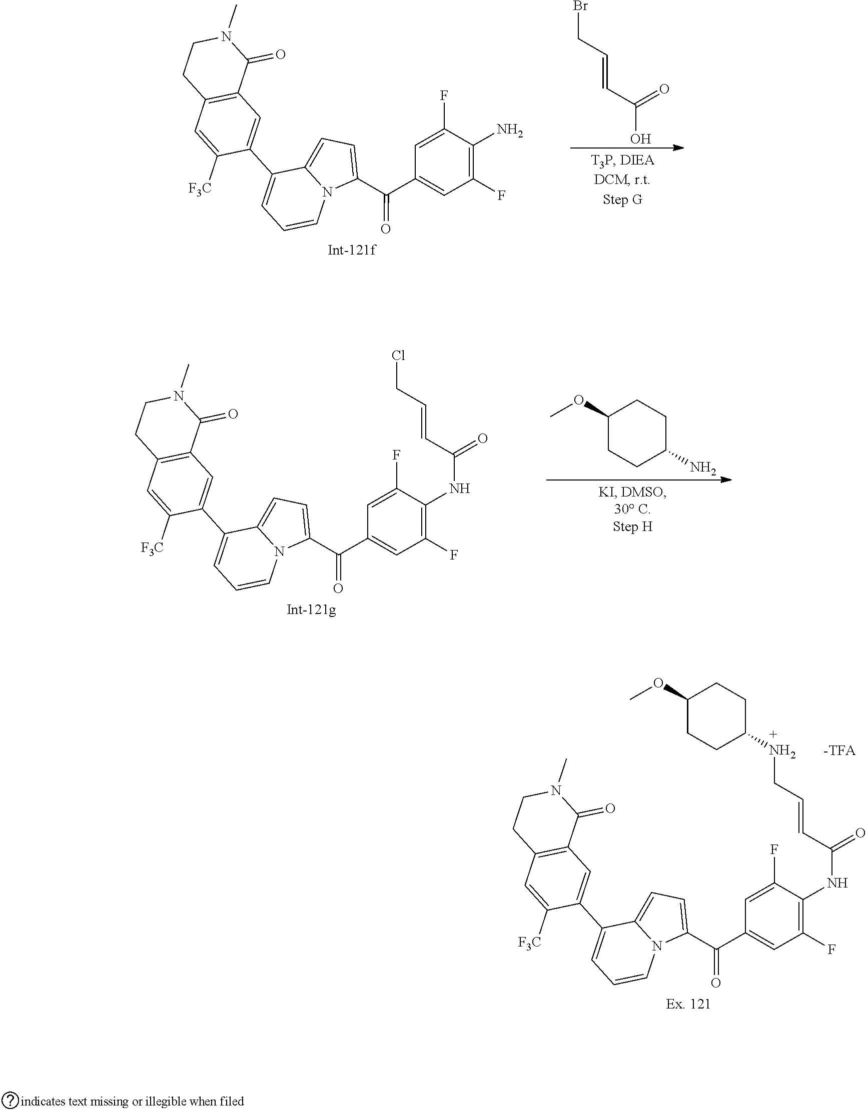

### Step A: methyl 2-amino-5-chloro-4-(trifluoromethyl) benzoate (Int-121a)

To a solution of methyl 2-amino-4-(trifluoromethyl) benzoate (10 g. 45.6 mmol) in DMF (100 mL) was added NCS (6.40 g, 47.9 mmol) at 25° C. The mixture was stirred at 70° C. for 2 h. The mixture was cooled, diluted with EtOAc (100 mL), washed with water (10 mL×3), dried over Na₂SO₄, and filtered, and the solvent was evaporated under reduced pressure to give the crude product. The crude product was purified by flash silica gel chromatography (ISCO®; 120 g SepaFlash® Silica Flash Column, Eluent of 5% ethyl acetate in petroleum ether gradient @ 25 mL/min) to give methyl 2-amino-5-chloro-4-(trifluoromethyl)benzoate (Int-121a). MS (ESI) [M+H+MeCN]⁺: m/z 295.0

### Step B: methyl 2-bromo-5-chloro-4-(trifluoromethyl) benzoate (Int-121b)

To a solution of methyl 2-amino-5-chloro-4-(trifluoromethyl) benzoate (2 g, 7.89 mmol) in MeCN (20 mL) was added copper(Il) bromide (5.28 g, 23.66 mmol). The mixture was stirred at 25° C. for 15 min and tert-butyl nitrite (1.626 g. 14.20 mmol. 90%) was added dropwise over 5 min. The reaction mixture was stirred at 25° C. for 30 min. then heated at 50° C. for 3 h. The mixture was concentrated in vacuo, and the residue was diluted with EtOAc (100 mL), and washed with water (20 mL) and brine (10 mL). The organic layer was dried over Na₂SO₄, and filtered, and the solvent was evaporated under reduced pressure to give the crude product. The crude product was purified by flash silica gel chromatography (ISCO®; 20 g SepaFlash® Silica Flash Column. Eluent of 4% ethyl acetate in petroleum ether gradient (a 25 mL/min) to give methyl 2-bromo-5-chloro-4-(trifluoromethyl)benzoate (Int-121b). ¹H NMR (400 MHz, CDCl₃) δ 7.98 (s, 1H), 7.92 (s, 1H), 3.98 (s, 3H).

### Step C: methyl (E)-5-chloro-2-(2-ethoxyvinyl)-4-(trifluoromethyl)benzoate (Int-121c)

To a solution of methyl 2-bromo-5-chloro-4-(trifluoromethyl)benzoate (2 g, 6.30 mmol) in 1,4-dioxane (20 mL) and water (6 mL) was added (E)-2-(2-ethoxyvinyl)-4,4,5,5-tetramethyl-1,3,2-dioxaborolane (1.77 g, 8.94 mmol), cesium carbonate (6.16 g. 18.90 mmol) and tetrakis(triphenylphosphine)palladium(0) (0.728 g, 0.630 mmol) at 25° C. under N2 atmosphere. The mixture was stirred at 90° C. for 3 h under N₂ atmosphere. LCMS showed the starting material was consumed and desired peak was formed. The mixture was cooled, diluted with EtOAc (100 mL), washed with water (20 mL), dried over Na₂SO₄, and filtered, and the solvent was evaporated under reduced pressure to give the crude product. The crude product was purified by flash silica gel chromatography (ISCO®; 20 g SepaFlash® Silica Flash Column, Eluent of 100% petroleum ether gradient @ 25 mL/min) to give methyl (E)-5-chloro-2-(2-ethoxyvinyl)-4-(trifluoromethyl)benzoate (Int-121c). MS (ESI) [M+H]⁺: m/z 309.0

### Step D: methyl 5-chloro-2-(2-oxoethyl)-4-(trifluoromethyl)benzoate (Int-121d)

To a solution of methyl (E)-5-chloro-2-(2-ethoxyvinyl)-4-(trifluoromethyl)benzoate (600 mg, 1.944 mmol) in THF (3 mL) was added aqueous HCl (3.24 mL, 19.44 mmol, 6M in water) at 25° C. The mixture was stirred at 25° C. for 1 h. The mixture was cooled, diluted with EtOAc (20 mL), washed with water (5 mL), dried over Na₂SO₄, and filtered, and the solvent was evaporated under reduced pressure to give the crude product. The crude product was purified by flash silica gel chromatography (ISCO®; 12 g SepaFlash®, Silica Flash Column, Eluent of 15% ethyl acetate in petroleum ether gradient @25 mL/min) to give methyl 5-chloro-2-(2-oxoethyl)-4-(trifluoromethyl)benzoate (Int-121d). MS (ESI) [M+H]⁺: m/z 281.0

### Step E: 7-chloro-2-methyl-6-(trifluoromethyl)-3,4-dihydroisoquinolin-1(2H)-one (Int-121e)

To a solution of methyl 5-chloro-2-(2-oxoethyl)-4-(trifluoromethyl)benzoate (500 mg, 1.782 mmol) in MeOH (5 mL) was added methylamine hydrochloride (601 mg. 8.91 mmol) at 25° C. The mixture was stirred at 25° C. for 30 min. Then to the reaction solution was added sodium cyanoborohydride (560 mg, 8.91 mmol), and the mixture was stirred at 25° C. for 15 h. The mixture was evaporated under reduced pressure to give the crude product. The crude product was purified by flash silica gel chromatography (ISCO®; 12 g SepaFlash® Silica Flash Column, Eluent of 8% ethyl acetate in petroleum ether gradient @ 25 mL/min) to give 7-chloro-2-methyl-6-(trifluoromethyl)-3,4-dihydroisoquinolin-1(2H)-one (Int-121e). MS (ESI) [M+H]⁺: m/z 264.0

### Step F: 7-(3-(4-amino-3,5-difluorobenzoyl)indolizin-8-yl)-2-methyl-6-(trifluoromethyl)-3,4-dihydroisoquinolin-1(2H)-one (Int-121f)

To a solution of (4-amino-3,5-difluorophenyl)(8-(4,4,5,5-tetramethyl-1,3,2-dioxaborolan-2-yl)indolizin-3-yl)methanone (54.4 mg, 0.137 mmol) in toluene (0.5 mL) was added 7-chloro-2-methyl-6-(trifluoromethyl)-3,4-dihydroisoquinolin-1(2H)-one (Int-121e)(30 mg, 0.114 mmol), chloro(2-dicyclohexylphosphino-2′,6′-dimethoxy-1,1′-biphenyl)[2-(2′-amino-1,1′-biphenyl)]palladium(II) (8.20 mg, 0.011 mmol) and potassium phosphate tribasic (0.228 mL, 0.228 mmol, 1M in water) at 25° C. under N₂ atmosphere. The mixture was stirred at 60° C. for 15 h under N₂ atmosphere. TLC (SiO₂; petroleum ether:ethyl acetate=1:1) showed starting material was consumed and new spots were observed. The mixture was cooled, diluted with EtOAc (10 mL), washed with water (3 mL), dried over Na₂SO₄, and filtered, and the solvent was evaporated under reduced pressure to give the crude product. The crude product was purified by preparative TLC (SiO₂, petroleum ether:ethyl acetate=1:1) to give 7-(3-(4-amino-3,5-difluorobenzoyl)indolizin-8-yl)-2-methyl-6-(trifluoromethyl)-3,4-dihydroisoquinolin-1(2H)-one (Int-121f). MS (ESI) [M+H]⁺: m/z 500.3

### Step G: (E)-4-chloro-N-(2,6-difluoro-4-(8-(2-methyl-1-oxo-6-(trifluoromethyl)-1,2,3,4-tetrahydroisoquinolin-7-yl)indolizine-3-carbonyl)phenyl)but-2-enamide (Int-121g)

To a solution of 7-(3-(4-amino-3,5-difluorobenzoyl)indolizin-8-yl)-2-methyl-6-(trifluoromethyl)-3,4-dihydroisoquinolin-1(2H)-one (50 mg, 0.100 mmol) (contained HCl) in DCM (1 mL) was added 4-bromocrotonic acid (132 mg, 0.801 mmol), 1-propanephosphonic anhydride solution (510 mg, 0.801 mmol) and N,N-diisopropylethylamine (129 mg, 1.001 mmol) at 25° C. The mixture was stirred at 25° C. for 15 h. The mixture was cooled, diluted with EtOAc (5 mL), washed with water (2 mL), dried over Na₂SO₄, and filtered, and the solvent was evaporated under reduced pressure to give the crude product (E)-4-chloro-N-(2,6-difluoro-4-(8-(2-methyl-1-oxo-6-(trifluoromethyl)-1,2,3,4-tetrahydroisoquinolin-7-yl)indolizine-3-carbonyl)phenyl)but-2-enamide (Int-121g). MS (ESI) [M+H]⁺: m/z 602.1

### Step H: (1r,4r)-N-((E)-4-((2,6-difluoro-4-(8-(2-methyl-1-oxo-6-(trifluoromethyl)-1,2,3,4-tetrahydroisoquinolin-7-yl)indolizine-3-carbonyl)phenyl)amino)-4-oxobut-2-en-1-yl)-4-methoxycyclohexan-1-aminium 2,2,2-trifluoroacetate (Ex. 121)

To a solution of (E)-4-chloro-N-(2,6-difluoro-4-(8-(2-methyl-1-oxo-6-(trifluoromethyl)-1,2,3,4-tetrahydroisoquinolin-7-yl)indolizine-3-carbonyl)phenyl)but-2-enamide (32 mg, 0.053 mmol) in DMSO (0.3 mL) was added (1r,4r)-4-methoxycyclohexan-1-amine (27.5 mg, 0.213 mmol) and KI (17.65 mg, 0.106 mmol) at 25° C. The mixture was stirred at 30° C. for 1 h. LCMS showed starting material was consumed and desired peak was formed. The mixture was evaporated under reduced pressure to give the crude product. The crude product was purified by reverse preparative HPLC (Column: Waters Xbridge BEH C18 100*25 mm*5 um; Condition: water (TFA)-ACN; Begin B-End B: 30--50; Gradient Time (min): 12: 100/B Hold Time (min): 2; FlowRate (mL/min): 25) to give (1r,4r)-N-((E)-4-((2,6-difluoro-4-(8-(2-methyl-1-oxo-6-(trifluoromethyl)-1,2,3,4-tetrahydroisoquinolin-7-yl)indolizine-3-carbonyl)phenyl)amino)-4-oxobut-2-en-1-yl)-4-methoxycyclohexan-1-aminium 2,2,2-trifluoroacetate (Ex. 121). MS (ESI) [M+H]⁺: m/z 695.2 ¹H NMR (400 MHz, MeOD) δ 9.96 (d, J=6.9 Hz, 1H), 8.0 (s, 1H), 7.85 (s, 1H), 7.52 (d, J=7.8 Hz, 2H), 7.43 (d, J=4.9 Hz, 1H), 7.23-7.26 (m, 1H), 7.18-7.22 (m, 1H), 6.93 (dt, J=15.3, 6.8 Hz, 1H), 6.59 (d, J=15.4 Hz, 1H), 6.17 (d, J=4.9 Hz, 1H), 3.94 (d, J=6.9 Hz, 2H), 3.74 (t, J=6.7 Hz, 2H), 3.36 (s, 3H), 3.20-3.25 (m, 3H), 3.13-3.19 (m, 4H), 2.20 (br d, J=10.4 Hz, 4H), 1.46 (q, J=11.6 Hz, 2H), 1.27-1.34 (m, 2H).

### Example 122

### (1r,4r)-N-((E)-4-((2,6-difluoro-4-(8-(2-methyl-3-oxo-6-(trifluoromethyl)isoindolin-5-yl)indolizine-3-carbonyl)phenylamino)-4-oxobut-2-en-1-yl)-4-methoxycyclohexan-1-aminium 2,2,2-trifluoroacetate

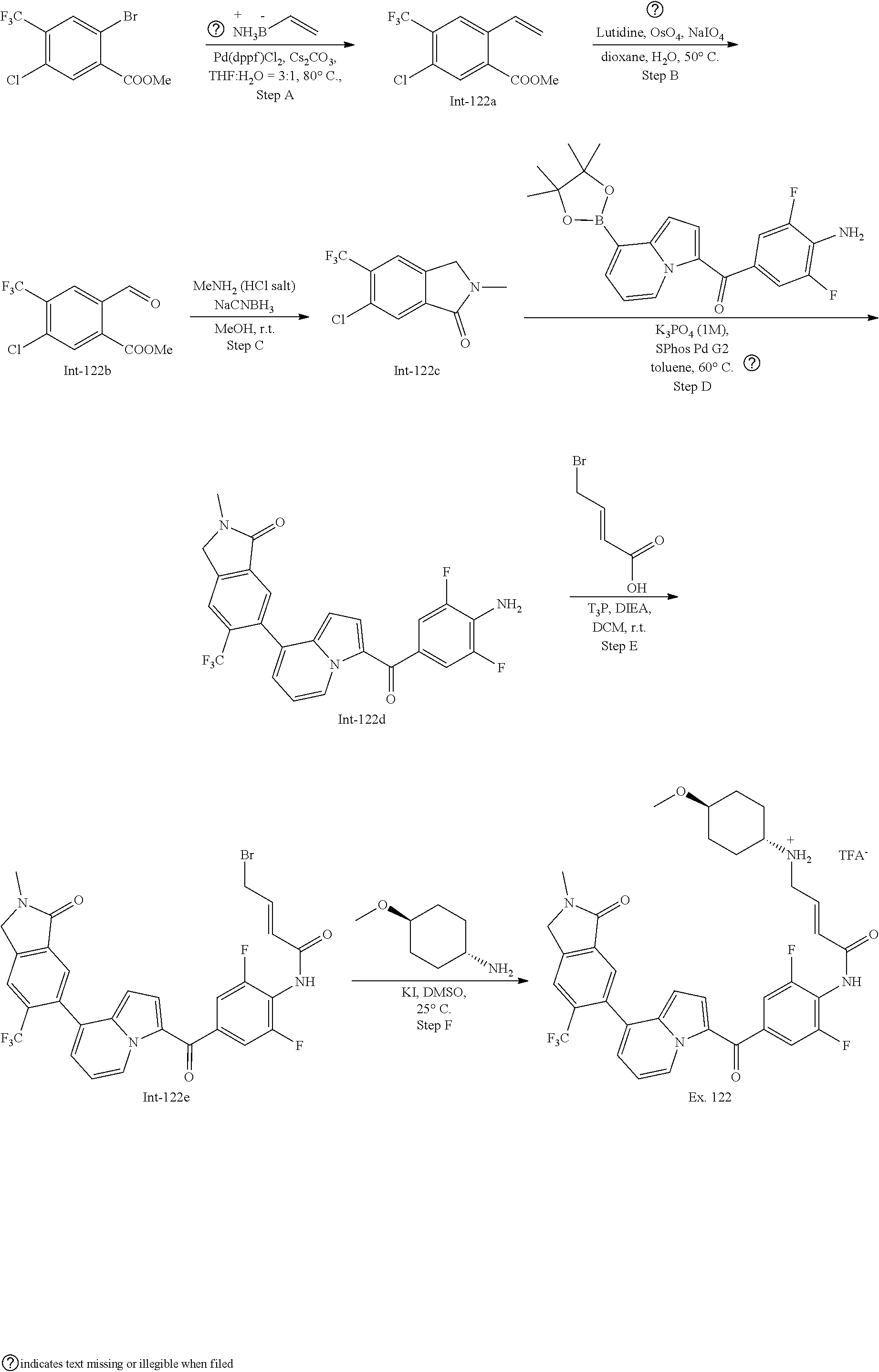

### Step A: methyl 5-chloro-4-(trifluoromethyl)-2-vinylbenzoate (Int-122a)

To a solution of methyl 2-bromo-5-chloro-4-(trifluoromethyl)benzoate (2 g, 6.30 mmol) in THF (18 mL) and water (6 mL) was added 1,1′-bis(diphenylphosphino)ferrocene-palladium(II)dichloride dichloromethane complex (0.514 g. 0.630 mmol), Cs₂CO₃ (6.16 g, 18.90 mmol) and poataaium vinyltrifluoroborate (0.844 g. 6.30 mmol) at 25° C. under N₂ atmosphere. The mixture was stirred at 80° C. for 15 h under N₂ atmosphere. The mixture was cooled, diluted with EtOAc (100 mL), washed with water (10 mL), dried over Na₂SO₄, and filtered, and the solvent was evaporated under reduced pressure to give the crude product. The crude product was purified by flash silica gel chromatography (ISCO®; 40 g SepaFlash® Silica Flash Column. Eluent of 5% ethyl acetate in petroleum ether gradient @ 25 mL/min) to give methyl 5-chloro-4-(trifluoromethyl)-2-vinylbenzoate (Int-122a). ¹H NMR (400 MHz, CDCl₃) δ 7.98 (s, 1H), 7.89 (s, 1H), 7.42 (dd, J=17.5, 11.0 Hz, 1H), 5.74 (d, J=17.5 Hz, 1H), 5.49 (d, J=11.1 Hz, 1H), 3.95 (s, 3H). F

### Step B: methyl 5-chloro-2-formyl-4-(trifluoromethyl)benzoate (Int-122b)

To a solution of methyl 5-chloro-4-(trifluoromethyl)-2-vinylbenzoate (500 mg, 1.889 mmol) in 1,4-dioxane (20 mL) and water (8 mL) was added 2,6-lutidine (405 mg. 3.78 mmol), osmium tetroxide (48.0 mg, 0.189 mmol) and sodium periodate (2425 mg. 11.34 mmol) at 25° C. The mixture was stirred at 25° C. for 1 h. The mixture was cooled, diluted with EtOAc (30 mL), washed with saturated sodium sulfite solution (8 mL), dried over Na₂SO₄, and filtered, and the solvent was evaporated under reduced pressure to give the crude product. The crude product was purified by flash silica gel chromatography (ISCO®; 12 g SepaFlash® Silica Flash Column. Eluent of 5% ethyl acetate in petroleum ether gradient @ 25 mL/min) to give methyl 5-chloro-2-formyl-4-(trifluoromethyl)benzoate (Int-122b). MS (ESI) [M+H]⁺: m/z 267.0

### Step C: 6-chloro-2-methyl-5-(trifluoromethyl)isoindolin-1-one (Int-122c)

To a solution of methyl 5-chloro-2-formyl-4-(trifluoromethyl)benzoate (100 mg. 0.375 mmol) in MeOH (1 mL) was added methylamine hydrochloride (127 mg, 1.875 mmol) at 25° C.; the mixture was stirred at 25° C. for 30 min. Then NaBH₃CN (118 mg. 1.875 mmol) was added to the solution, and the mixture was stirred at 25° C. for 15 h. The reaction solution was purified by preparative TLC (SiO₂, petroleum ether:ethyl acetate=3:1) to give 6-chloro-2-methyl-5-(trifluoromethyl)isoindolin-1-one (Int-122c). MS (ESI) [M+H]⁺: m/z 249.9

### Step D: 6-(3-(4-amino-3,5-difluorobenzoyl)indolizin-8-yl)-2-methyl-5-(trifluoromethyl)isoindolin-1-one (Int-122d)

To a solution of 6-chloro-2-methyl-5-(trifluoromethyl)isoindolin-1-one (40 mg, 0.160 mmol) in toluene (1 mL) was added (4-amino-3,5-difluorophenyl)(8-(4,4,5,5-tetramethyl-1,3,2-dioxaborolan-2-yl)indolizin-3-yl)methanone (77 mg, 0.192 mmol). SPhos Pd G2 (11.55 mg, 0.016 mmol) and potassium phosphate tribasic (0.32 mL, 0.320 mmol, 1 M in H₂O) at 25° C. under N₂ atmosphere. The mixture was stirred at 60° C. for 15 h. The mixture was cooled, diluted with EtOAc (2 mL), washed with H₂O (0.5 mL), dried over Na₂SO₄, and filtered, and the solvent was evaporated under reduced pressure. The residue was purified by preparative TLC (SiO₂, petroleum ether:ethyl acetate=2:1) to give 6-(3-(4-amino-3,5-difluorobenzoyl)indolizin-8-yl)-2-methyl-5-(trifluoromethyl)isoindolin-1-one (Int-122d). MS (ESI) [M+H]⁺: m/z 486.1

### Step E: (E)-4-bromo-N-(2,6-difluoro-4-(8-(2-methyl-3-oxo-6-(trifluoromethyl)isoindolin-5-yl)indolizine-3-carbonyl)phenyl)but-2-enamide (Int-122e)

To a solution of 6-(3-(4-amino-3,5-difluorobenzoyl)indolizin-8-yl)-2-methy-5-(trifluoromethyl)isoindolin-1-one (30 mg, 0.062 mmol) in DCM (0.5 mL) was added (E)-4-bromobut-2-enoic acid (40.8 mg, 0.247 mmol). DIEA (0.130 mL, 0.742 mmol) and 1-propanephosphonic anhydride solution (157 mg, 0.247 mmol. 50% wt in EtOAc) at 25° C. The mixture was stirred at 25° C. for 15 h. Then (E)-4-bromobut-2-enoic acid (40.8 mg. 0.247 mmol). DIEA (0.130 mL, 0.742 mmol) and 1-propanephosphonic anhydride solution (157 mg, 0.247 mmol, 50% wt in EtOAc) were added to the solution, and the mixture was stirred at 25° C. for 2 h. The mixture was diluted with DCM (3 mL), washed with H₂O (1 mL), dried over Na₂SO₄, and filtered, and the solvent was evaporated under reduced pressure. The residue was purified by preparative TLC (SiO₂, petroleum ether:ethyl acetate=2:1) to give (E)-4-bromo-N-(2,6-difluoro-4-(8-(2-methyl-3-oxo-6-(trifluoromethyl)isoindolin-5-yl)indolizine-3-carbonyl)phenyl)but-2-enamide (Int-122e). MS (ESI) [M+H]⁺: m/z 631.9, 633.9

### Step F: (1r,4r)-N-((E)-4-((2,6-difluoro-4-(8-(2-methyl-3-oxo-6-(trifluoromethyl)isoindolin-5-yl)indolizine-3-carbonyl)phenyl)amino)-4-oxobut-2-en-1-yl)-4-methoxycyclohexan-1-aminium 2,2,2-trifluoroacetate (Ex. 122)

To a solution of (E)-4-bromo-N-(2,6-difluoro-4-(8-(2-methyl-3-oxo-6-(trifluoromethyl)isoindolin-5-yl)indolizine-3-carbonyl)phenyl)but-2-enamide (20 mg, 0.032 mmol) in DMSO (1 mL) was added KI (15.75 mg, 0.095 mmol) and (1r,4r)-4-methoxycyclohexan-1-amine (4.90 mg, 0.038 mmol) at 25° C. The mixture was stirred at 25° C. for 2 h. The reaction solution was purified by reverse preparative HPLC (Column: Waters Xbridge BEH C 18 100*25 mm*5 um; Condition: water (TFA)-ACN; Begin B-End B: 26-46; Gradient Time (min): 12; 100% B Hold Time (min): 2: Flow Rate (mL/min): 25) to give (1r,4r)-N-((E)-4-((2,6-difluoro-4-(8-(2-methyl-3-oxo-6-(trifluoromethyl)isoindolin-5-yl)indolizine-3-carbonyl)phenyl)amino)-4-oxobut-2-en-1-yl)-4-methoxycyclohexan-1-aminium 2,2,2-trifluoroacetate (Ex. 122). MS (ESI) [M+H]⁺: m/z 681.2 ¹H NMR (400 MHz, MeOD) δ 9.99 (d, J=7.0 Hz, 1H), 8.18 (s, 1H), 7.85 (s, 1H), 7.54 (d, J=7.9 Hz, 2H), 7.45 (d, J=4.9 Hz, 1H), 7.30 (d, J=7.0 Hz, 1H), 7.21-7.27 (m, 1H), 6.94 (dt, J=15.4, 6.8 Hz, 1H), 6.61 (d, J=15.4 Hz, 1H), 6.17 (d, J=4.9 Hz, 1H), 4.72 (s, 2H), 3.96 (d, J=7.3 Hz, 2H), 3.38 (s, 3H), 3.28 (s, 3H), 3.21-3.26 (m, 1H), 3.15-3.21 (m, 1H), 2.22 (br d, J=12.1 Hz, 4H), 1.43-1.53 (m, 2H), 1.29-1.39 (m, 2H).

### Example 123

### (E)-N-(4-((2,6-difluoro-4-(8-(7-hydroxy-2,3-dimethyl-5-(trifluoromethyl)-2H-indazol-6-yl)indolizine-3-carbonyl)phenyl)amino)-4-oxobut-2-en-1-yl)-3-methyloxetan-3-aminium 2,2,2-trifluoroacetate

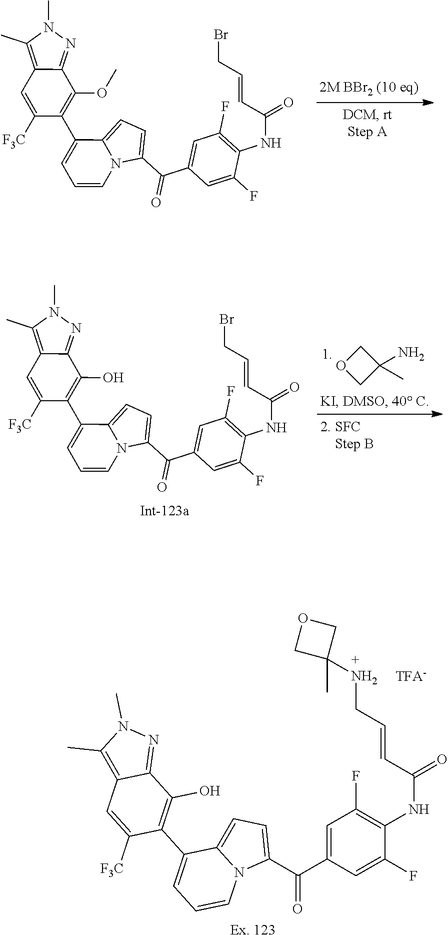

### Step A: (E)-4-bromo-N-(2,6-difluoro-4-(8-(7-hydroxy-2,3-dimethyl-5-(trifluoromethyl)-2H-indazol-6-yl)indolizine-3-carbonyl)phenyl)but-2-enamide (Int-123a)

To a solution of (E)-4-bromo-N-(2,6-difluoro-4-(8-(7-methoxy-2,3-dimethyl-5-(trifluoromethyl)-2H-indazol-6-yl)indolizine-3-carbonyl)phenyl)but-2-enamide (90 mg. 0.136 mmol) in CH₂Cl₂ (1.8 mL) was added BBr₃ (0.680 mL, 1.361 mmol) at 30° C. The mixture was stirred at 30° C. for 1 h. The mixture was quenched with sat.aq.NaHCO₃ (2 mL), extracted with DCM (3×3 mL), dried over Na₂SO₄, and filtered, and the solvent was evaporated under reduced pressure to give (E)-4-bromo-N-(2,6-difluoro-4-(8-(7-hydroxy-2,3-dimethyl-5-(trifluoromethyl)-2H-indazol-6-yl)indolizine-3-carbonyl)phenyl)but-2-enamide (Int-123a). MS (ESI) [M+H]⁺: m/z 647.0, 649.0

### Step B: (E)-N-(4-((2,6-difluoro-4-(8-(7-hydroxy-2,3-dimethyl-5-(trifluoromethyl)-2H-indazol-6-yl)indolizine-3-carbonyl)phenyl)amino)-4-oxobut-2-en-1-yl)-3-methyloxetan-3-aminium 2,2,2-trifluoroacetate (Ex. 123)

To a solution of (E)-4-bromo-N-(2,6-difluoro-4-(8-(7-hydroxy-2,3-dimethyl-5-(trifluoromethyl)-2H-indazol-6-yl)indolizine-3-carbonyl)phenyl)but-2-enamide (100 mg, 0.154 mmol) in DMSO (2 mL) was added 3-methyloxetan-3-amine hydrochloride (95 mg, 0.772 mmol). DIEA (0.08 mL) and potassium iodide (51.3 mg, 0.309 mmol) at 25° C. under N₂ atmosphere. The mixture was stirred at 40° C. for 1 h. The mixture was cooled, diluted with water (5 mL), extracted with DCM (5×2 mL), dried over Na₂SO₄, and filtered, and the solvent was evaporated under reduced pressure to give the crude product. The crude product was purified by reverse preparative HPLC (Column: YMC-Actus Triart C18 150*30 mm*5 um; Condition: water (0.1% TFA)-MeCN; Begin B--End B: 40-60; Gradient Time (min): 10.5; 100% B Hold Time (min): 1.5; FlowRate (mL/min): 25) to give the racemic product (60 mg) as yellow solid.

The racemate was separated by preparative SFC (Column:DAICEL CHIRALCEL OD (250 mm*30 mm. 10 um); Condition:0.1% NH₃H₂O EtOH; Begin B--End B: 55--55; Flow Rate 80 mL/min) followed by repurify by Prep-HPLC (Cloumn:Boston Green ODS 150*30 mm*5 um; Condition: water (TFA)-ACN; Gradient Time (min):10; Begin B--End B: 28--58: Flow Rate 25 mL/min) to (E)-N-(4-((2,6-difluoro-4-(8-(7-hydroxy-2,3-dimethyl-5-(trifluoromethyl)-2H-indazol-6-yl)indolizine-3-carbonyl)phenyl)amino)-4-oxobut-2-en-1-yl)-3-methyloxetan-3-aminium 2,2,2-trifluoroacetate (Ex. 123, the first eluting isomer from SFC). MS (ESI) [M+H]⁺: m/z 654.1 ¹H NMR (400 MHz, MeOD) δ 9.96 (br d, J=6.8 Hz, 1H), 7.73 (s, 1H), 7.50 (br d, J=8.1 Hz, 2H), 7.39-7.35 (m, 1H), 7.31-7.26 (m, 1H), 7.24-7.14 (m, 1H), 7.04-6.89 (m, 1H), 6.65 (br d, J=15.2 Hz, 1H), 6.15 (d, J=4.6 Hz, 1H), 4.80 (br d, J=7.8 Hz, 2H), 4.58 (d, J=7.8 Hz, 2H), 4.15 (s, 3H), 3.97 (br d, J=6.8 Hz, 2H), 2.70 (d, J=1.7 Hz, 3H), 1.74 (s, 3H).

### Example 124

### (1r,4r)-N-((E)-4-((4-(8-(4-chloro-2-methyl-6-(trifluoromethyl)-2H-indazol-5-yl)indolizine-3-carbonyl)-2,6-difluorophenyl)amino)-4-oxobut-2-en-1-yl)-4-methoxycyclohexan-1-aminium 2,2,2-trifluoroacetate

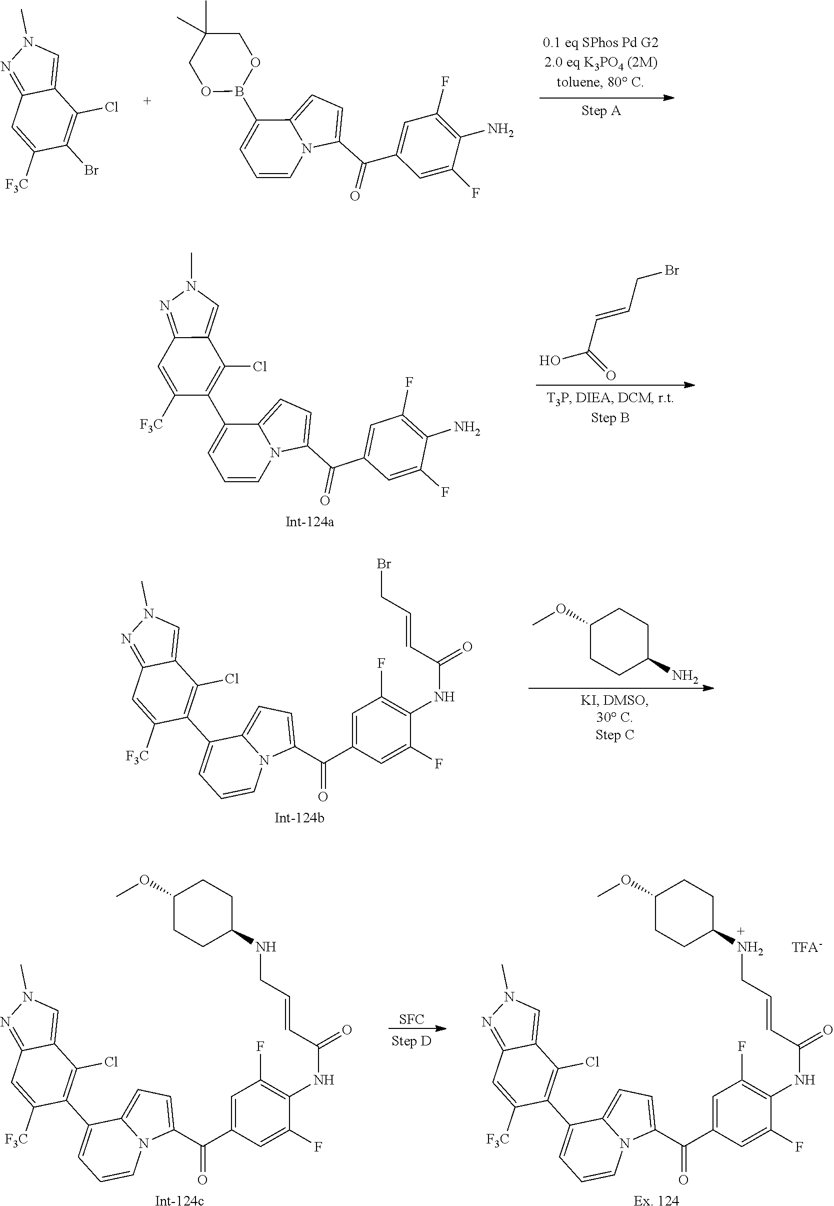

### Step A: (4-amino-3,5-difluorophenyl)(8-(4-chloro-2-methyl-6-(trifluoromethyl)-2H-indazol-5-yl)indolizin-3-yl)methanone (Int-124a)

A mixture of 5-bromo-4-chloro-2-methyl-6-(trifluoromethyl)-2H-indazole (100 mg, 0.319 mmol), (4-amino-3,5-difluorophenyl)(8-(5,5-dimethyl-1,3,2-dioxaborinan-2-yl)indolizin-3-yl)methanone (135 mg, 0.351 mmol), SPhos Pd G2 (22.99 mg, 0.032 mmol) and 2M aqueous K₃PO₄ (0.319 mL, 0.638 mmol) in toluene (1.5 mL) was degassed and backfilled with N₂ 3 times. The reaction mixture was stirred at 80° C. for 1.5 h. The reaction mixture was extracted with EtOAc (20 mL×3). The combined organic layers were washed with brine (20 mL), dried over MgSO₄ and filtered. The filtrate was concentrated under reduced pressure to give the residue which was purified by pre-TLC (SiO₂, Pet. ether/EtOAc=2:1) to give (4-amino-3,5-difluorophenyl)(8-(4-chloro-2-methyl-6-(trifluoromethyl 1)-2H-indazol-5-yl)indolizin-3-yl)methanone (Int-124a). MS (ESI) [M+H]⁺: m/z 505.1

### Step B: (E)-4-bromo-N-(4-(8-(4-chloro-2-methyl-6-(trifluoromethyl)-2H-indazol-5-yl)indolizine-3-carbonyl)-2,6-difluorophenyl)but-2-enamide (Int-124b)

To a solution of (4-amino-3,5-difluorophenyl)(8-(4-chloro-2-methyl-6-(trifluoromethyl)-2H-indazol-5-yl)indolizin-3-yl)methanone (63 mg, 0.125 mmol) in DCM (2 mL) was added (E)-4-bromobut-2-enoic acid (61.8 mg, 0.374 mmol), I-propanephosphonic anhydride solution (318 mg, 0.499 mmol) (50% in EtOAc) and DIEA (0.218 mL, 1.248 mmol). The reaction mixture was stirred at 25° C. for 16 h. The reaction mixture was quenched with water (2 mL) and extracted with DCM (10 mL×3). The combined organic layers were washed with brine (15 mL), dried over Na₂SO₄ and filtered. The filtrate was concentrated under reduced pressure to give the residue which was purified by Prep-TLC (SiO₂, Pet. ether/EtOAc=2:1, v/v) to give (E)-4-bromo-N-(4-(8-(4-chloro-2-methyl-6-(trifluoromethyl)-2H-indazol-5-yl)indolizine-3-carbonyl)-2,6-difluorophenyl)but-2-enamide (Int-124b). MS (ESI) [M+H]⁺: m/z 651.1, 653.1

### Step C: (E)-N-(4-(8-(4-chloro-2-methyl-6-(trifluoromethyl)-2H-indazol-5-yl)indolizine-3-carbonyl)-2,6-difluorophenyl)-4-(((1r,4r)-4-methoxycyclohexyl)amino)but-2-enamide (Int-124c)

To a solution of (E)-4-bromo-N-(4-(8-(4-chloro-2-methyl-6-(trifluoromethyl)-2H-indazol-5-yl)indolizine-3-carbonyl)-2,6-difluorophenyl)but-2-enamide (62 mg, 0.095 mmol) and KI (31.6 mg, 0.190 mmol) in DMSO (0.5 mL) was added (1r,4r)-4-methoxycyclohexan-1-amine (24.58 mg, 0.190 mmol). The reaction mixture was stirred at 30° C. for 15 h. The reaction mixture was extracted with EtOAc (10 mL×3). The combined organic layers were washed with brine (15 mL), dried over Na₂SO₄ and filtered. The filtrate was concentrated under reduced pressure to give the residue which was purified by Prep-TLC (SiO₂, DCM/MeOH=10:1, v/v) to give (E)-N-(4-(8-(4-chloro-2-methyl-6-(trifluoromethyl)-2H-indazol-5-yl)indolizine-3-carbonyl)-2,6-difluorophenyl)-4-(((1r,4r)-4-methoxycyclohexyl)amino)but-2-enamide (Int-124c). MS (ESI) [M+H]⁺: m/z 700.2

### Step D: (1r,4r)-N-((E)-4-((4-(8-(4-chloro-2-methyl-6-(trifluoromethyl)-2H-indazol-5-yl)indolizine-3-carbonyl)-2,6-difluorophenyl)amino)-4-oxobut-2-en-1-yl)-4-methoxycyclohexan-1-aminium 2,2,2-trifluoroacetate (Ex. 124)

The racemic (E)-N-(4-(8-(4-chloro-2-methyl-6-(trifluoromethyl)-2H-indazol-5-yl)indolizine-3-carbonyl)-2,6-difluorophenyl)-4-(((1r,4r)-4-methoxycyclohexyl)amino)but-2-enamide (43 mg, 0.061 mmol) was separated by preparative SFC (Column DAICEL CHIRALPAK IG (250 mm*30 mm, 10 um) Condition 0.1% NH₃H₂O EtOH Begin B 60% End B 60% Gradient Time (min) 100% B Hold Time (min) FlowRate (mL/min) 80 Injections 30) to give (1r,4r)-N-((E)-4-((4-(8-(4-chloro-2-methyl-6-(trifluoromethyl)-2H-indazol-5-yl)indolizine-3-carbonyl)-2,6-difluorophenyl)amino)-4-oxobut-2-en-1-yl)-4-methoxycyclohexan-1-aminium 2,2,2-trifluoroacetate (Ex. 124, the second eluting isomer from SFC). MS (ESI) [M+H]⁺: m/z 700.1 ¹H NMR (400 MHz, MeOD) δ 10.01 (d, 3=6.9 Hz, 1H), 8.55 (s, 1H), 8.20 (s, 1H), 7.53 (d, J=7.9 Hz, 2H), 7.43 (d, J=4.9 Hz, 1H), 7.36-7.29 (m, 1H), 7.28-7.21 (m, 1H), 6.94 (td, J=6.8, 15.3 Hz, 1H), 6.61 (d, J=15.4 Hz, 1H), 6.12 (d, 3=4.8 Hz, 1H), 4.36 (s. 3H), 3.96 (br d, J=6.7 Hz, 2H), 3.38 (s, 3H), 3.29-3.07 (m, 2H), 2.22 (br d, J=9.2 Hz, 4H), 1.62-1.41 (m, 2H), 1.40-1.20 (m, 2H).

### Example 125

### (1r,4r)-N-((E)-4-((2,6-difluoro-4-(8-(2-methyl-1-oxo-6-(trifluoromethyl)-1,2-dihydroisoquinolin-7-yl)indolizine-3-carbonyl)phenyl)amino)-4-oxobut-2-en-1-yl)-4-methoxycyclohexan-1-aminium 2,2,2-trifluoroacetate

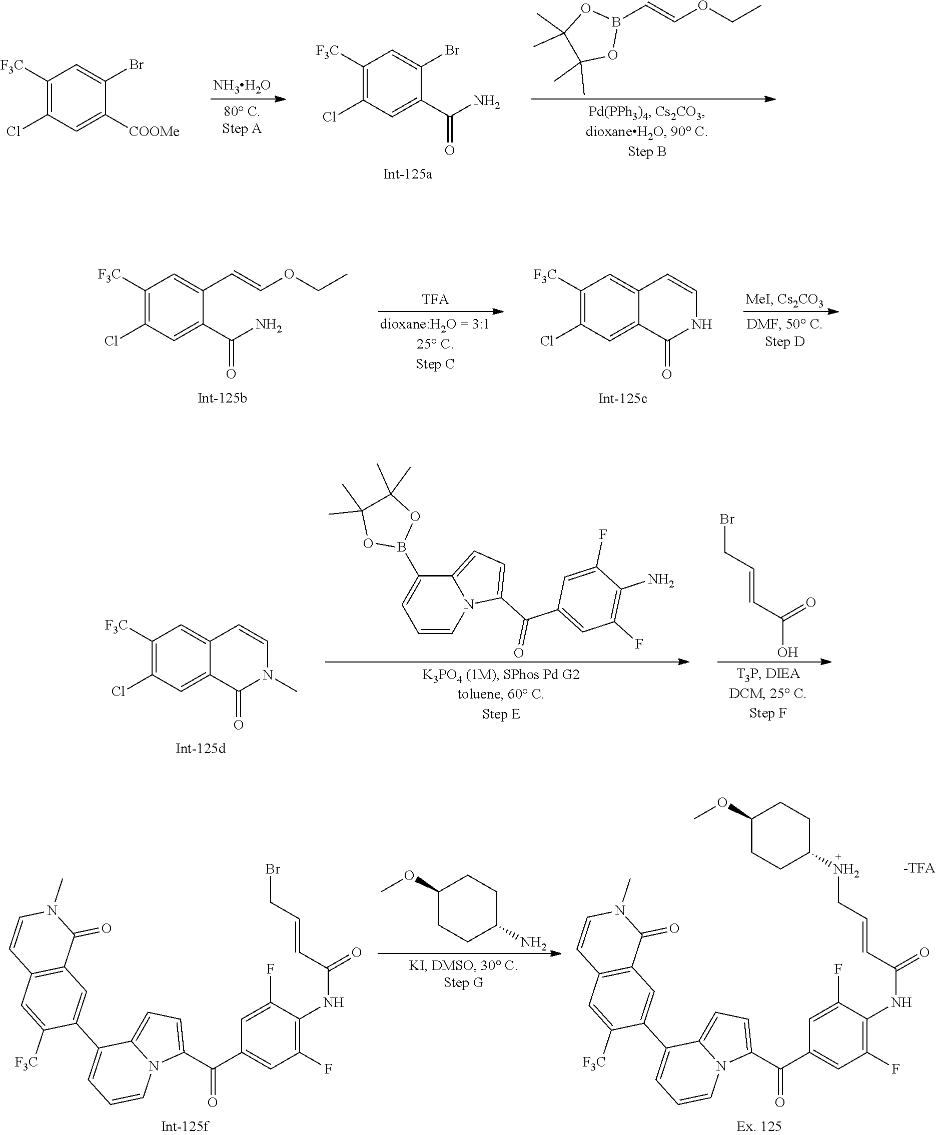

### Step A: 2-bromo-5-chloro-4-trifluoromethyl)benzamide (Int-125a)

To a solution of methyl 2-bromo-5-chloro-4-(trifluoromethyl) benzoate (2 g. 6.30 mmol) was added concentrated ammonium hydroxide solution (20 mL, 130 mmol) at 25° C. The mixture was stirred at 80° C. for 15 h. The mixture was cooled and evaporated under reduced pressure to give the crude product. The crude product was purified by flash silica gel chromatography (ISCO®; 20 g SepaFlash® Silica Flash Column. Eluent of 30 ethyl acetate in petroleum ether gradient @ 25 mL/min) to give 2-bromo-5-chloro-4-(trifluoromethyl)benzamide (Int-125a). MS (ESI) [M+H+MeCN]⁺: m/z 343, 345.

### Step B: (E)-5-chloro-2-(2-ethoxyvinyl)-4-(trifluoromethyl)benzamide (Int-125b)

To a solution of 2-bromo-5-chloro-4-(trifluoromethyl)benzamide (580 mg, 1.918 mmol) in 1,4-dioxane (6 mL) and water (2 mL) was added (E)-1-ethoxyethene-2-boronic acid pinacol ester (949 mg, 4.79 mmol), tetrakis(triphenylphosphine) palladium(0) (222 mg, 0.192 mmol) and Cs₂CO₃ (1874 mg, 5.75 mmol) at 25° C. under N₂ atmosphere. The mixture was stirred at 90° C. for 3 h under N₂ atmosphere. The mixture was cooled, diluted with EtOAc (50 mL), washed with water (10 mL), dried over Na₂SO₄, and filtered, and the solvent was evaporated under reduced pressure to give the crude product. The crude product was purified by flash silica gel chromatography (ISCO®; 20 g SepaFlash® Silica Flash Column. Eluent of 10% ethyl acetate in petroleum ether gradient @z 25 mL/min) to give (E)-5-chloro-2-(2-ethoxyvinyl)-4-(trifluoromethyl)benzamide (Int-125b). MS (ESI) [M+H]⁺: m/z 294.

### Step C: 7-chloro-6-(trifluoromethyl)isoquinolin-1(2H)-one (Int-125c)

To a solution of (E)-5-chloro-2-(2-ethoxyvinyl)-4-trifluoromethyl)benzamide (490 mg, 1.669 mmol) in 1,4-dioxane (6 mL) and water (2 mL) was added TFA (1 mL. 1.669 mmol) at 0° C. The mixture was stirred at 25° C. for 3 h. The mixture was cooled, diluted with EtOAc (30 mL), washed with water (10 mL), dried over Na₂SO₄, and filtered, and the solvent was evaporated under reduced pressure to give the crude product. The crude product was purified by flash silica gel chromatography (ISCO®; 4 g SepaFlash® Silica Flash Column. Eluent of 30% ethyl acetate in petroleum ether gradient @ 25 mL/min) to give 7-chloro-6-(trifluoromethyl)isoquinolin-1(2H)-one (Int-125c). MS (ESI) [M+H+MeCN]⁺: m/z 289.

### Step D: 7-chloro-2-methyl-6-(trifluoromethyl) isoquinolin-1(2H)-one (Int-125d)

To a solution of 7-chloro-6-(trifluoromethyl) isoquinolin-1(2H)-one (150 mg. 0.606 mmol) in DMF (1.5 mL) was added Cs₂CO₃ (296 mg, 0.909 mmol) and Mel (0.057 mL, 0.909 mmol) at 25° C. The mixture was stirred at 50° C. for 3 h. The mixture was cooled, diluted with EtOAc (50 mL), washed with water (5×2 mL), dried over Na₂SO₄, and filtered, and the solvent was evaporated under reduced pressure to give the crude product. The crude product was purified by flash silica gel chromatography (ISCO®; 4 g SepaFlash® Silica Flash Column, Eluent of 20% ethyl acetate in petroleum ether gradient @ 25 mL/min) to give 7-chloro-2-methyl-6-(trifluoromethyl)isoquinolin-1(2H)-one (Int-125d). MS (ESI) [M+H]⁺: m/z 262.

### Step E: 7-(3-(4-amino-3,5-difluorobenzoyl)indolizin-8-yl)-2-methyl-6-(trifluoromethyl)isoquinolin-1(21)-one (Int-125e)

To a solution of (4-amino-3,5-difluorophenyl)(8-(4,4,5,5-tetramethyl-1,3,2-dioxaborolan-2-yl)indolizin-3-yl)methanone (200 mg, 0.502 mmol) in toluene (2 mL) was added 7-chloro-2-methyl-6-(trifluoromethyl)isoquinolin-1(2H)-one (158 mg, 0.603 mmol), chloro(2-dicyclohexylphosphino-2′,6-dimethoxy-1,1′-biphenyl)[2-(2′-amino-1,1′-biphenyl)]palladium(II) (36.2 mg, 0.050 mmol) and potassium phosphate tribasic (0.095 mL, 0.143 mmol. 1 M in H₂O) at 25° C. under N₂ atmosphere. The mixture was stirred at 60° C. for 12 h. The mixture was cooled, diluted with EtOAc (20 mL), washed with H₂O (5 mL), dried over Na₂SO₄, and filtered, and the solvent was evaporated under reduced pressure to give the crude product. The crude product was purified by flash silica gel chromatography (ISCO®; 4 g SepaFlash® Silica Flash Column. Eluent of 20% ethyl acetate in petroleum ether gradient @ 25 mL/min) to give 7-(3-(4-amino-3,5-difluorobenzoyl)indolizin-8-yl)-2-methyl-6-(trifluoromethyl)isoquinolin-1(2H)-one (Int-125e). MS (ESI) [M+H]⁺: m/z 498.

### Step F: (E)-4-bromo-N-(2,6-difluoro-4-(8-(2-methyl-1-oxo-6-(trifluoromethyl)-1,2-dihydroisoquinolin-7-yl)indolizine-3-carbonyl)phenyl)but-2-enamide (Int-125f)

To a solution of 7-(3-(4-amino-3,5-difluorobenzoyl)indolizin-8-yl)-2-methyl-6-(trifluoromethyl)isoquinolin-1(2H)-one (20 mg, 0.040 mmol) in DCM (0.5 mL) was added 4-bromocrotonic acid (53.1 mg, 0.322 mmol). 1-propanephosphonic anhydride solution (102 mg, 0.322 mmol) and N,N-diisopropylethylamine (52.0 mg, 0.402 mmol) at 25° C. The mixture was stirred at 25° C. for 15 h. The mixture was cooled, diluted with EtOAc (6 mL), washed with water (2 mL), dried over Na₂SO₄, and filtered, and the solvent was evaporated under reduced pressure to give the crude product (E)-4-bromo-N-(2,6-difluoro-4-(8-(2-methyl-1-oxo-6-(trifluoromethyl)-1,2-dihydroisoquinolin-7-yl)indolizine-3-carbonyl)phenyl)but-2-enamide (Int-125f). MS (ESI) [M+H]⁺: m/z 644, 646.

### Step G: (1r,4r)-N-((E)-4-((2,6-difluoro-4-(8-(2-methyl-1-oxo-6-(trifluoromethyl)-1,2-dihydroisoquinolin-7-yl)indolizine-3-carbonyl)phenyl)amino)-4-oxobut-2-en-1-yl)-4-methoxycyclohexan-1-aminium 2,2,2-trifluoroacetate (Ex. 125)

To a solution of (E)-4-bromo-N-(2,6-difluoro-4-(8-(2-methyl-1-oxo-6-(trifluoromethyl)-1,2-dihydroisoquinolin-7-yl)indolizine-3-carbonyl)phenyl)but-2-enamide (25 mg, 0.039 mmol) in DMSO (0.2 mL) was added (1r,4r)-4-methoxycyclohexan-1-amine (20.05 mg, 0.155 mmol) and KI (12.88 mg, 0.078 mmol) at 25° C. The mixture was stirred at 30° C. for 2 h. The mixture was evaporated under reduced pressure to give the crude product. The crude product was purified by reverse preparative HPLC (Column: Waters Xbridge BEH C18 100*25 mm*5 um; Condition: water (TFA)-ACN; Begin B--End B: 25-45; Gradient Time (min): 12: 100% B Hold Time (min): 2; FlowRate (mL/min): 25) to give (1r,4r)-N-((E)-4-((2,6-difluoro-4-(8-(2-methyl-1-oxo-6-(trifluoromethyl)-1,2-dihydroisoquinolin-7-yl)indolizine-3-carbonyl)phenyl)amino)-4-oxobut-2-en-1-yl)-4-methoxycyclohexan-1-aminium 2,2,2-trifluoroacetate (Ex. 125). MS (ESI) [M+H]⁺: m/z 693. ¹H NMR (400 MHz, MeOD) δ 9.98 (d, J=7.0 Hz, 1H), 8.38 (s, 1H), 8.25 (s, 1H), 7.58 (d, J=7.3 Hz, 1H), 7.52 (d, J=7.9 Hz, 2H), 7.43 (d, J=4.9 Hz, 1H), 7.31 (d, J=7.0 Hz, 1H), 7.17-7.25 (m, 1H), 6.88-6.96 (m, 2H), 6.59 (d, J=15.4 Hz, 1H), 6.18 (d, J=4.9 Hz, 1H), 3.94 (d, J=6.7 Hz, 2H), 3.66 (s, 2H), 3.64-3.67 (m, 1H), 3.36 (s, 3H), 3.21-3.26 (m, 1H), 3.13-3.19 (m, 1H), 2.20 (br d, J=10.1 Hz, 4H), 1.40-1.51 (m, 2H), 1.31 (br d, J=12.8 Hz, 2H).

### Example 126

### (E)-N-(4-(4-(4-chloro-1,2-dimethyl-6-(trifluoromethyl)-1H-benzo[d]imidazol-5-yl)-1H-indole-1-carbonyl)-2,6-difluorophenyl)-4-(((1r,4r)-4-methoxycyclohexyl)amino)but-2-enamide

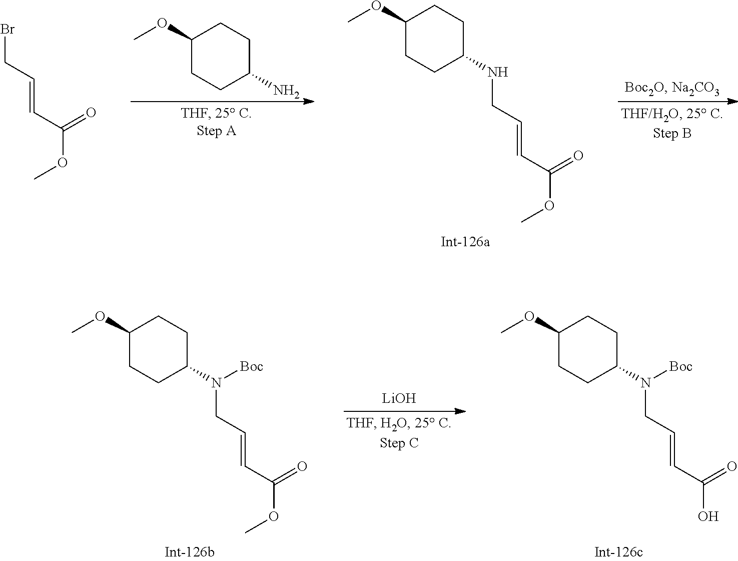

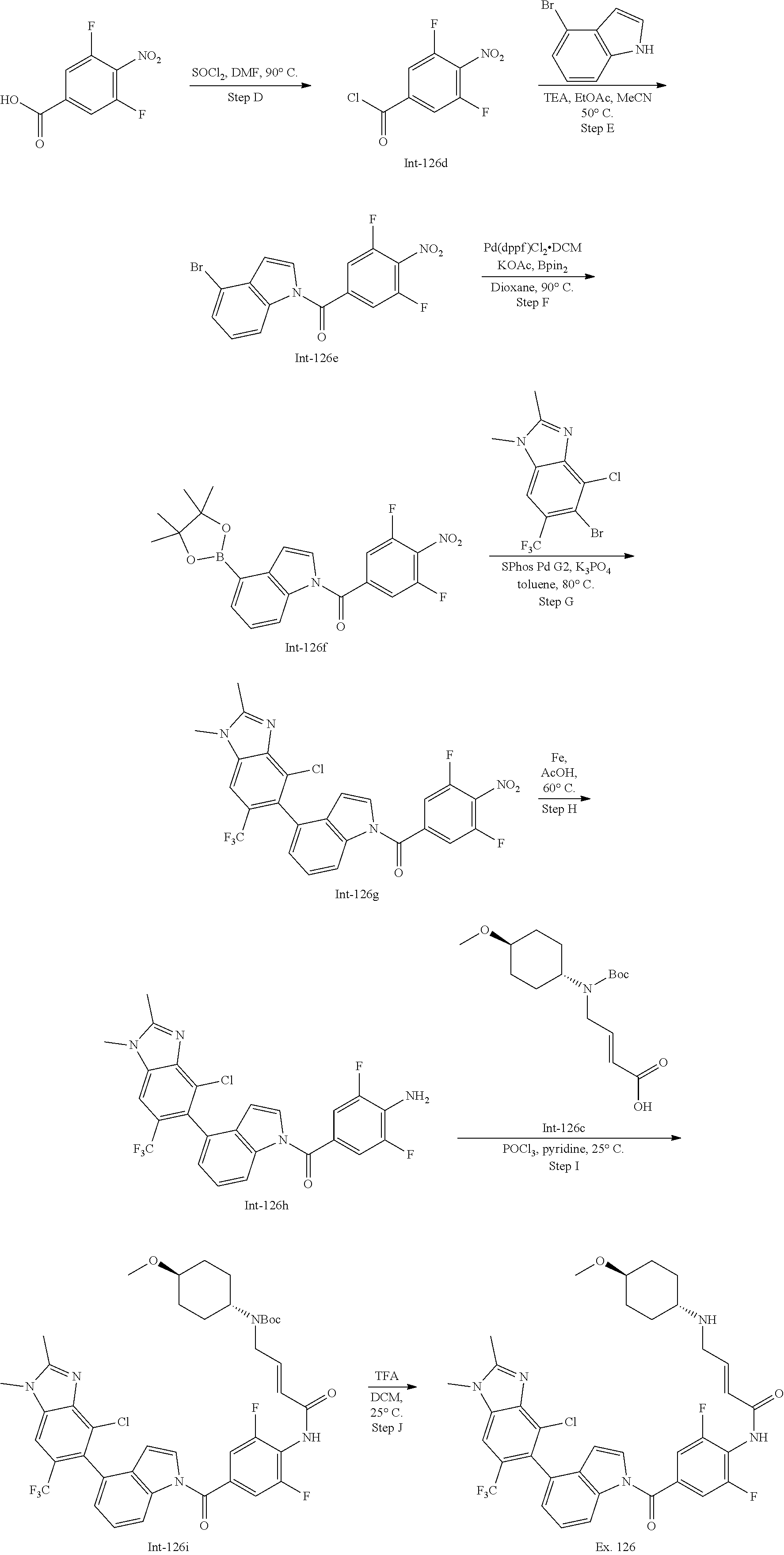

### Step A: methyl (E)-4-(((1r,4r)-4-methoxycyclohexyl)amino)but-2-enoate (Int-126a)

To a solution of (1r,4r)-4-methoxycyclohexan-1-amine (144 mg, 1.12 mmol) in THF (1.5 mL) was added methyl (E)-4-bromobut-2-enoate (100 mg, 0.559 mmol). The reaction mixture was stirred at 25° C. for 12 h. The reaction mixture was concentrated under reduced pressure to remove most solvent. The residue was diluted with DCM (8 mL). The organic layer was washed with brine (1 mL×2), dried over Na₂SO₄ and filtered. The filtrate was concentrated under reduced pressure to give methyl (E)-4-(((1r,4r)-4-methoxycyclohexyl)amino)but-2-enoate (Int-126a). MS (ESI) [M+H]⁺: m/z 228.

### Step B: methyl (E)-4-((tert-butoxycarbonyl)((1r,4r)-4-methoxycyclohexyl)amino)but-2-enoate (Int-126b)

To a solution of methyl (E)-4-(((1r,4r)-4-methoxycyclohexyl)amino)but-2-enoate (127 mg, 0.559 mmol) and sodium carbonate (118 mg, 1.12 mmol) in THF (1 mL) and H₂O (1 mL) was added (Boc)₂O (0.26 mL, 1.12 mmol). The reaction mixture was stirred at 25° C. for 4 h. The reaction mixture was diluted with EtOAc (5 mL) and H₂O (5 mL).

The aqueous layer was separated and extracted with EtOAc (5 mL×3). The combined organic layers were dried over Na₂SO₄ and filtered. The filtrate was concentrated under reduced pressure to give the residue which was purified by flash silica gel chromatography (ISCO®; 4 g Agela® Silica Flash Column, Eluent of 0˜16% EtOAc/Pet. ether gradient @ 30 mL/min) to give methyl (IE)-4-((tert-butoxycarbonyl)((1r,4r)-4-methoxycyclohexyl)amino)but-2-enoate (Int-126b). MS (ESI) [M+H]⁺: m/z 328.

### Step C: (E)-4-((tert-butoxycarbonyl)((1r,4r)-4-methoxycyclohexyl)amino)but-2-enoic acid (Int-126c)

To a solution of methyl (E)-4-((tert-butoxycarbonyl)((1r,4r)-4-methoxycyclohexyl)amino)but-2-enoate (170 mg, 0.519 mmol) in THF (1 mL) and H₂O (1 mL) was added lithium hydroxide monohydrate (87.0 mg, 2.08 mmol). The reaction mixture was stirred at 25° C. for 3 h. The reaction mixture was diluted with H₂O (1 mL) and EtOAc (1 mL). The mixture was acidified by citric acid until pH 7. The reaction mixture was concentrated to remove most solvent. The residue was purified by Prep-HPLC (Column Waters Xbridge BEH C18 100*25 mm*5 um, Condition water (TFA)-ACN Begin B 23. End B 43 Gradient Time (min) 12, 100% B Hold Time (min) 2 FlowRate (mL/min) 25) to give (E)-4-((tert-butoxycarbonyl)((1r,4r)-4-methoxycyclohexyl)amino)but-2-enoic acid (Int-126c). MS (ESI) [M+H]⁺: m/z 314.

### Step D: 3,5-difluoro-4-nitrobenzoyl chloride (Int-126d)

To a solution of 3,5-difluoro-4-nitrobenzoic acid (0.90 g, 4.43 mmol) in thionyl chloride (10 mL, 137 mmol) was added DMF (0.03 mL, 0.443 mmol). The reaction mixture was stirred at 90° C. for 1 h. The reaction mixture was concentrated under reduced pressure to give 3,5-difluoro-4-nitrobenzoyl chloride (Int-126d) which was used in the next step without further purification.

### Step E: (4-bromo-1H-indol-1-yl)(3,5-difluoro-4-nitrophenyl)methanone (Int-126e)

To a solution of 4-bromo-1H-indole (450 mg, 2.30 mmol) in EtOAc (2.5 mL) and MeCN (2.5 mL) were added TEA (1.92 mL, 13.8 mmol). DMAP (84.0 mg, 0.689 mmol) and 3,5-difluoro-4-nitrobenzoyl chloride (915 mg, 4.13 mmol). The mixture was stirred at 50° C. for 12 h. The reaction mixture was concentrated to give the residue which was purified by flash silica gel chromatography (ISCO®; 12 g Agela® Silica Flash Column, Eluent of 0˜5% EtOAc/Pet. ether gradient @ 30 mL/min) to give (4-bromo-1H-indol-1-yl)(3,5-difluoro-4-nitrophenyl)methanone (Int-126e). ¹H NMR (400 MHz, CDCl3) δ 8.35 (d, J=8.34 Hz, 1H), 7.55 (d, J=7.87 Hz, 111), 7.46-7.52 (m, 2H), 7.32 (t. J=799 Hz, 1H), 7.22 (d, J=4.22 Hz, 1H), 6.82 (d, J=3.93 Hz, 1H),

### Step F: (3,5-difluoro-4-nitrophenyl)(4-(4,4,5,5-tetramethyl-1,3,2-dioxaborolan-2-yl)-1H-indol-1-yl)methanone (Int-126f)

A mixture of (4-bromo-1H-indol-1-yl)(3,5-difluoro-4-nitrophenyl)methanone (294 mg, 0.771 mmol). 1,1′-bis(diphenylphosphino)ferrocene-palladium(II)dichloride dichloromethane complex (63.0 mg, 0.077 mmol), bis(pinacolato)diboron (294 mg, 1.16 mmol) and potassium acetate (227 mg, 2.31 mmol) in dioxane (1.5 mL) was bubbled with N2 for 1 min. The reaction mixture was stirred at 90° C. for 4 h. The reaction mixture was diluted with EtOAc (5 mL) and filtered. The filtrate was concentrated under reduced pressure to give the residue which was purified by flash silica gel chromatography (ISCO®; 4 g Agela® Silica Flash Column. Eluent of 0˜12% EtOAc/Pet. ether gradient @ 30 mL/min. dry loaded) to give (3,5-difluoro-4-nitrophenyl)(4-(4,4,5,5-tetramethyl-1,3,2-dioxaborolan-2-yl)-1H-indol-1-yl)methanone (int-126f). MS (ESI) [M+H]⁺: m/z 429.

### Step G: (4-(4-chloro-1,2-dimethyl-6-(trifluoromethyl)-1H-benzo[d]imidazol-5-yl)-1H-indol-1-yl)(3,5-difluoro-4-nitrophenyl)methanone (Int-126g)

A mixture of 5-bromo-4-chloro-1,2-dimethyl-6-(trifluoromethyl)-1H-benzo[d]imidazole (127 mg, 0.388 mmol), (3,5-difluoro-4-nitrophenyl)(4-(4,4,5,5-tetramethyl-1,3,2-dioxaborolan-2-yl)-1H-indol-1-yl)methanone (183 mg, 0.427 mmol), potassium phosphate tribasic (0.78 mL, 0.776 mmol) (IM in water) and (2-dicyclohexylphosphino-2′,6′-dimethoxybiphenyl) [2-(2′-amino-1,1′-biphenyl)]palladium(II) methanesulfonate (30.3 mg, 0.039 mmol) in toluene (4 mL) was bubbled with N₂ for 1 min. The reaction mixture was stirred at 80° C. for 12 h. The reaction mixture was diluted with EtOAc (5 mL) and H₂O (3 mL). The aqueous layer was separated and extracted with EtOAc (5 mL×3). The combined organic layers were washed with brine (5 mL), dried over Na₂SO₄ and filtered. The filtrate was concentrated under reduced pressure to give the residue which was purified by preparatory TLC (SiO₂, Pet. ether/EtOAc=2:1, v/v) to give (4-(4-chloro-1,2-dimethyl-6-(trifluoromethyl)-1H-benzo[d]imidazol-5-yl)-1H-indol-1-yl)(3,5-difluoro-4-nitrophenyl)methanone (int-126g). MS (ESI) [M+H]⁺: m/z 549.

### Step H: (4-amino-3,5-difluorophenyl)(4-(4-chloro-1,2-dimethyl-6-(trifluoromethyl)-1H-benzo[d]imidazol-5-yl)-1H-indol-1-yl)methanone (Int-126h)

To a solution of (4-(4-chloro-1,2-dimethyl-6-(trifluoromethyl)-1H-benzo[d]imidazol-5-yl)-1H-indol-1-yl)(3,5-difluoro-4-nitrophenyl)methanone (72.4 mg, 0.132 mmol) in AcOH (1.5 mL) was added iron powder (73.7 mg, 1.32 mmol). The reaction mixture was stirred at 60° C. for 1 h. The reaction mixture was filtered. The filtrate was concentrated under reduced pressure to give the residue which was purified by Prep-TLC (SiO₂. Pet. ether/Acetone=2:1, v/v) to give (4-amino-3,5-difluorophenyl)(4-(4-chloro-1,2-dimethyl-6-(trifluoromethyl)-1H-benzo[d]imidazol-5-yl)-1H-indol-1-yl)methanone (Int-126h). MS (ESI) [M+H]⁺: m/z 519.

### Step I: tert-butyl ((P)-4-((4-(4-(4-chloro-1,2-dimethyl-6-(trifluoromethyl)-1H-benzo[d]imidazol-5-yl)-1H-indole-1-carbonyl)-2,6-difluorophenyl)amino)-4-oxobut-2-en-1-yl)((1r,4r)-4-methoxycyclohexyl)carbamate (Int-126i)

To a solution of (4-amino-3,5-difluorophenyl)(4-(4-chloro-1,2-dimethyl-6-(trifluoromethyl)-1H-benzo[d]imidazol-5-yl)-1H-indol-1-yl)methanone (50.0 mg, 0.096 mmol) and (f)-4-((tert-butoxycarbonyl)((1r,4r)-4-methoxycyclohexyl)amino)but-2-enoic acid (60.4 mg, 0.193 mmol) in pyridine (0.5 mL) was added POCl₃ (15 mg, 0.096 mmol). The reaction mixture was stirred at 25° C. for 1 h. The reaction was quenched with H₂O (2 mL) and EtOAc (2 mL). The aqueous layer was separated and extracted with EtOAc (2 mL×3). The combined organic layers were washed with brine (2 mL), dried over Na₂SO₄ and filtered. The filtrate was concentrated under reduced pressure to give the residue which was purified by Prep-TLC (SiO₂. Pet. ether/EtOAc=1:1, v/v) to give tert-butyl ((E)-4-((4-(4-(4-chloro-1,2-dimethyl-6-(trifluoromethyl)-1H-benzo[d]imidazol-5-yl)-1H-indole-1-carbonyl)-2,6-difluorophenyl)amino)-4-oxobut-2-en-1-yl)((1r,4r)-4-methoxycyclohexyl)carbamate (Int-126i). MS (ESI) [M+H]⁺: m/z 814.

### Step J: (E)-N-(4-(4-(4-chloro-1,2-dimethyl-6-(trifluoromethyl)-1H-benzo[d]imidazol-5-yl)-1H-indole-1-carbonyl)-2,6-difluorophenyl)-4-(((1r,4r)-4-methoxycyclohexyl)amino)but-2-enamide (Ex. 126)

To a solution of tert-butyl ((E)-4-((4-(4-(4-chloro-1,2-dimethyl-6-(trifluoromethyl)-1H-benzo[d]imidazol-5-yl)-1H-indole-1-carbonyl)-2,6-difluorophenyl)amino)-4-oxobut-2-en-1-yl)((1r,4r)-4-methoxycyclohexyl)carbamate (42.0 mg, 0.052 mmol) in DCM (1.0 mL) was added TFA (0.5 mL). The reaction mixture was stirred at 25° C. for 30 min. The reaction mixture was concentrated under reduced pressure (25° C.) to give the residue which was purified by Prep-HPLC (Column Waters Xbridge BEH C18 100*25 mm*5 um, Condition water (FA)-ACN Begin B 12. End B 32 Gradient Time (min) 12, 100% B Hold Time (min) 2 FlowRate (mL/min) 25. Injections 1) to give (E)-N-(4-(4-(4-chloro-1,2-dimethyl-6-(trifluoromethyl)-1H-benzo[d]imidazol-5-yl)-1H-indole-1-carbonyl)-2,6-difluorophenyl)-4-(((1r,4r)-4-methoxycyclohexyl)amino)but-2-enamide (Ex. 126). MS (ESI) [M+H]⁺: m/z 714.2 ¹H NMR (400 MHz, CDCl₃) δ 8.50 (d, 3=8.34 Hz, 1H), 7.72 (s, 1H), 7.50 (t, J=7.87 Hz, 1H), 7.41 (d, J=7.39 Hz, 2H), 7.22-7.26 (m, 2H), 7.11 (td, J=5.10, 15.32 Hz, 1H), 6.31 (d, J=15.38 Hz, 1H), 6.14 (d, J=3.70 Hz, 1H), 3.88 (s, 3H), 3.55 (d, J=4.41 Hz, 2H), 3.35 (s. 3H), 3.08-3.19 (m, 1H), 2.74 (s, 3H), 2.57-2.67 (m, 1H), 2.10 (d, J=9.30 Hz, 2H), 2.01-2.04 (m, 2H), 1.19-1.30 (m, 4H).

### Example 127

### mono((E)-4-chloro-5-(3-(4-(4-((2,2-dimethylcyclobutyl)ammonio)but-2-enamido)-3,5-difluorobenzoyl)indolizin-8-yl)-1,2-dimethyl-6-(trifluoromethyl)-1H-benzo[d]imidazol-3-ium) mono(2,2,2-trifluoroacetate)

### Step 1

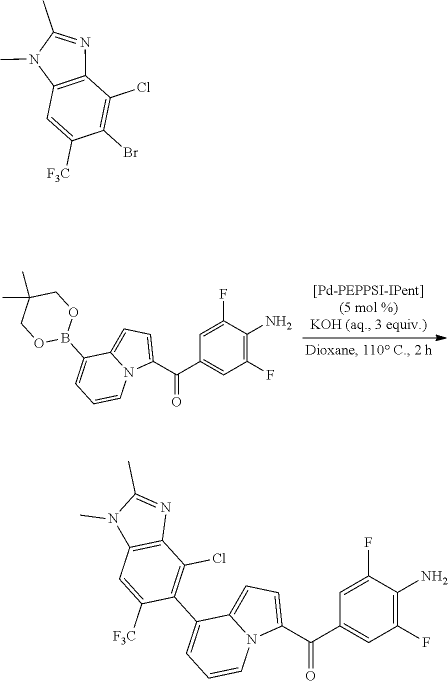 [SMILES: Cc1nc2c(Cl)c(Br)c(C(F)(F)F)cc2n1C ; CC1(C)COB(c2cccn3c(C(=O)c4cc(F)c(N)c(F)c4)ccc23)OC1 ; Cc1nc2c(Cl)c(-c3cccn4c(C(=O)c5cc(F)c(N)c(F)c5)ccc34)c(C(F)(F)F)cc2n1C]

5-bromo-4-chloro-1,2-dimethyl-6-(trifluoromethyl)-1H-benzo[d]imidazole (900 mg, 2.75 mmol), (4-amino-3,5-difluorophenyl)(8-(5,5-dimethyl-1,3,2-dioxaborinan-2-yl)indolizine-3-yl)methanone (2111 mg, 5.50 mmol), and potassium hydroxide (463 mg, 8.24 mmol) was dissolved in dioxane (27.5 mL), and dichloro[1,3-bis(2,6-di-3-pentylphenyl)indolizin-2-ylidene](3-chloropyridyl)palladium(II) (65.4 mg, 0.082 mmol) was added and the reaction heated to 110° C. for 2 hours, split up between two 40 mL microwave vials.

The vials were removed from heat and cooled to room temperature and combined. The reaction mixture was diluted with 100 mL water, and the solid formed was collected on a frit. The solid was dried under vacuum. The dried solid was transferred to an Erlenmeyer flask, and 15 mL EtOAc and 4 mL THF were added. The slurry was stirred for 30 minutes, and the insoluble material was filtered away. The filtrate was concentrated and loaded onto a 120 g silica gel column eluting from 0-10% methanol in CH₂Cl₂ to yield desired product as a yellow oil.

The purified material was resolved by SFC ((R,R)-Whelk-O, 21×250 mm, 5 um; Conditions: 40% MeOH with 0.1% NH₄OH) to afford peak 2, the second-luting isomer, as (4-amino-3,5-difluorophenyl)(8-(4-chloro-1,2-dimethyl-6-(trifluoromethyl)-1H-benzo[d]indolizin-5-yl)indolizine-3-yl)methanone.

### Step 2

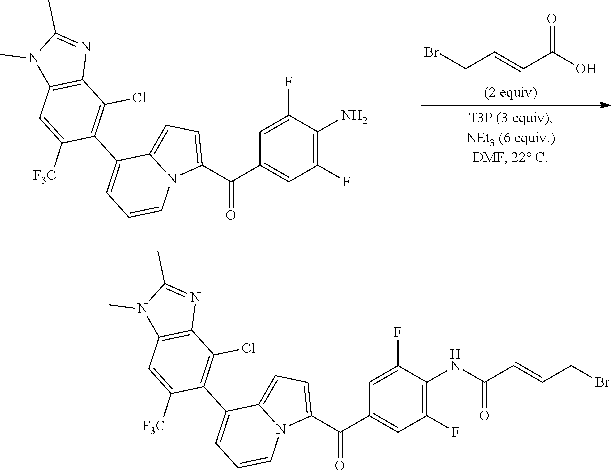

(E)-4-bromobut-2-enoic acid (137 mg, 0.829 mmol) and T3P (50% wt in EtOAc) (740 μl, 1.243 mmol) were added to a solution of (4-amino-3,5-difluorophenyl)(8-(4-chloro-1,2-dimethyl-6-(trifluoromethyl)-1H-benzo[d]imidazol-5-yl)indolizin-3-yl)methanone (215 mg, 0.414 mmol) in DMF (2072 μl). The reaction mixture was stirred for 20 minutes. Triethylamine (347 μl, 2.486 mmol) was added and the reaction was stirred for an additional 2 hours. 10 mL of water were added, followed by 15 mL EtOAc. The biphasic mixture was separated, and the organic layer was washed with water (10 mL) and brine (10 mL), dried over MgSO₄, filtered, and concentrated under vacuum. The crude residue was purified by silica gel column chromatography (0-10% MeOH in DCM) to yield (E)-4-bromo-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide.

### Step 3

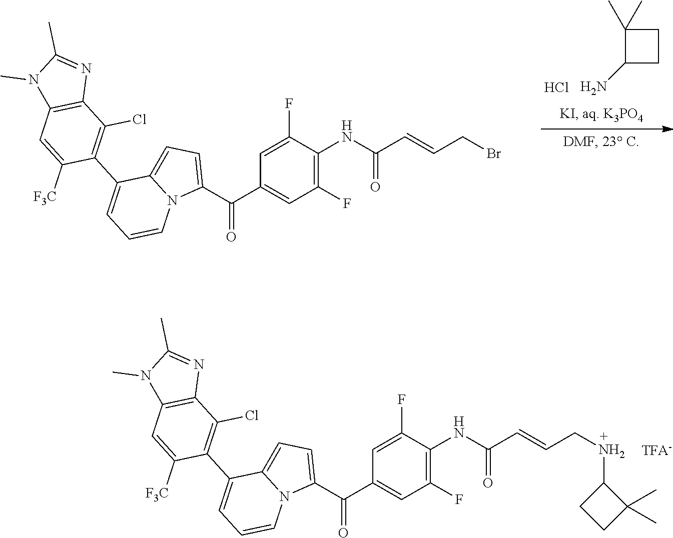

A vial was loaded with 2,2-dimethylcyclobutan-1-amine, HCl salt (8.14 mg, 0.06 mmol) and 60 μL of 0.316 M solution of (b)-4-bromo-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide (13.3 mg, 0.02 mmol) in DMF. Over this mixture, potassium iodide (10 mg, 0.06 mmol) was added, followed by a solution of tripotassium phosphate (1M. 0.06 mmol. 60 μL) via syringe. The reaction was stirred for 2.5 hours at 23° C. The reaction mixture was filtered and purified by HPLC under TFA modifier conditions. MS (ESI): [M+H]⁺ m/z: 684, 1H NMR (499 MHz, DMSO-d6) δ 10.35 (s, 1H), 9.92 (t, J=3.8 Hz, 1H), 8.95 (br. s, 1H), 8.71 (br. s. 1H), 8.20 (s, 1H), 7.60 (d, J=7.9 Hz, 2H), 7.42 (d, J=4.7 Hz, 1H), 7.31 (d, J=4.0 Hz, 2H), 6.91 (dt, J=15.5, 4.9 Hz, 1H), 6.53 (d, J=15.3 Hz, 1H), 6.01 (d, J=4.6 Hz, 1H), 3.92 (s, 3H), 3.77 (br. s, 2H), 2.65 (s, 3H). 2.17-2.00 (m, 1H), 1.98-1.59 (m, 1H), 1.62-1.59 (m, 1H), 1.25 (s, 3H), 1.17 (s, 3H).

### Examples 128,129 and 130

Ex. 128 to 130 below were made according to a similar procedure, using (E)-4-bromo-N-(4-(8-(4-chloro-1,2-dimethyl-6-(trifluoromethyl)-1H-benzo[d]imidazol-5-yl)indolizine-3-carbonyl)-2,6-difluorophenyl)but-2-enamide as starting material.

| | | | [M + H]⁺ |
|---|---|---|---|
| Int | Structure | Compound Name | Found |
| Ex. 128 | 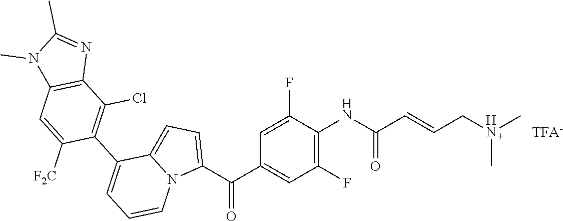 | mono((E)-4-chloro-5-(3-(4-(4- (dimethylammonio)but-2- enamido)-3,5- difluorobenzoyl)indolizin-8- yl)-1,2-dimethyl-6- (trifluoromethyl)-1H- benzo[d]imidazol-3-ium) mono(2,2,2-trifluoroacetate) | 630 |
| Ex. 129 | 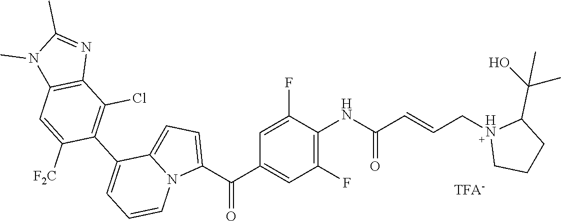 | mono((E)-4-chloro-5-(3-(3,5- difluoro-4-(4-(2-(2- hydroxypropan-2- yl)pyrrolidin-1-ium-1-yl)but- 2-enamido)benzoyl)indolizin- 8-yl)-1,2-dimethyl-6- (trifluoromethyl)-1H- benzo[d]imidazol-3-ium) mono(2,2,2-trifluoroacetate) | 714 |
| Ex. 130 | 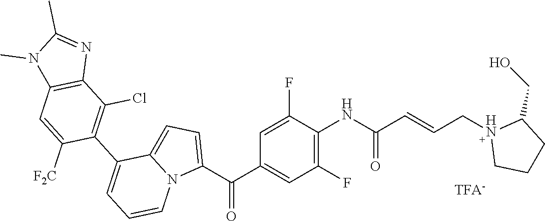 | mono(4-chloro-5-(3-(3,5- difluoro-4-((E)-4-((2S)-2- (hydroxymethyl)pyrrolidin-1- ium-1-yl)but-2- enamido)benzoyl)indolizin-8- yl)-1,2-dimethyl-6- (trifluoromethyl)-1H- benzo[d]imidazol-3-ium) mono(2,2,2-trifluoroacetate) | 686 |

### Example 131

### (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((3-methyltetrahydrofuran-3-yl)amino)but-2-enamide (Ex. 131a and Ex. 131b)

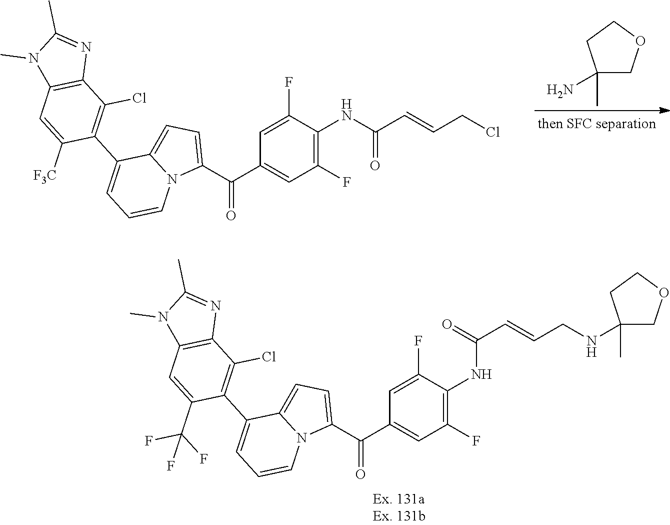

The procedure of Example 127 was performed except that 3-methyloxolan-3-amine was used, thereby obtaining (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((3-methyltetrahydrofuran-3-yl)amino)but-2-enamide. The crude mixture was purified by SFC (OD-H, 21×250 mm, 5 um; Conditions: 30% MeOH with 0.1% NH₄OH) to afford the desired compound as a mixture of two peaks (first eluting peak).

The mixture was resolved by an additional SFC purification (CCO F4, 21×250 mm, 5 um; Conditions: 40% IPA with 0.1% NH₄OH) to yield (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((3-methyltetrahydrofuran-3-yl)amino)but-2-enamide; Peak 1 (Ex. 131a). MS (ESI) [M+H]⁺: m/z 686.1; 1H NMR (499 MHz, DMSO-d6) δ 10.00 (s, 1H), 9.92 (t, J=3.8 Hz, 1H), 8.20 (s, 1H), 7.56 (d, J=7.9 Hz, 2H), 7.43 (d, J=4.7 Hz 1H), 7.31 (d, J=4.0 Hz, 2H), 6.91 (dt, J=15.5, 4.9 Hz, 1H), 6.38 (d, J=15.3 Hz, 1H), 6.00 (d, J=4.6 Hz, 1H), 3.92 (s, 3H), 3.85 (q, J=7.8 Hz, 1H), 3.74 (q, J=8.0 Hz, 1H), 3.54 (d, J=8.2 Hz, 2H), 3.36 (d, J=7.6 Hz, 3H), 2.66 (s, 3H), 1.89 (dt, J=13.7, 7.0 Hz, 1H), 1.73-1.63 (m, 1H), 1.21 (s, 3H). And (E)-N-(4-(8-(4-chloro-1,2-dimethyl-6-(trifluoromethyl)-1H-benzo[d]imidazol-5-yl)indolizine-3-carbonyl)-2,6-difluorophenyl)-4-((3-methyltetrahydrofuran-3-yl)amino)but-2-enamide; Peak 2 (Ex. 131b). MS (ESI) [M+H]⁺: m/z 686.1; 1H NMR (499 MHz, DMSO-d6) δ 10.02 (s, 1H), 9.92 (s, 1H), 8.21 (s, 1H), 7.57 (d, J=7.4 Hz, 2H), 7.43 (s, 1H), 7.31 (s, 2H), 6.91 (d, J=15.9 Hz, 1H), 6.38 (d, J=15.1 Hz, 1H), 6.00 (s, 1H), 3.92 (s, 3H), 3.84 (s, 1H), 3.75 (s, 1H), 3.54 (d, J=7.7 Hz, 2H), 3.38 (br. s. 3H), 2.66 (s, 3H), 1.89 (s, 1H), 1.69 (s, 1H), 1.21 (s, 3H).

### Example 132

### (3S)—N-((E)-4-((2,6-difluoro-4-(8-(1-hydroxy-4-methyl-2,3-dihydro-1H-inden-5-indolizine-3-carbonyl)phenyl)amino)-4-oxobut-2-en-1-vi)tetrahydrofuran-3-aminium 2,2,2-trifluoroacetate

### Step 1

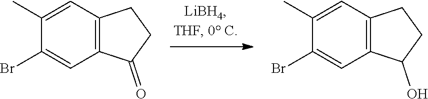

A vial was loaded with 6-bromo-5-methyl-1-indanone (161.5 mg, 0.718 mmol) and dissolved in 1 mL of THF. The solution was cooled to 0° C. with an ice bath and lithium borohydride was added (1 mL, 2.15 mmol). After 30 min. the ice bath was removed, and the reaction was allowed to stir at room temperature for 1.5 hours. The reaction was quenched with 2 mL of a NH₄Cl aqueous solution and extracted with DCM 3×5 mL, dried over Na₂SO₄ and evaporated under reduced pressure. The compound was used in the next step without further purification. 6-bromo-5-methyl-2,3-dihydro-1H-inden-1-ol was obtained. ¹H NMR (499 MHz, Chloroform-d) δ 7.55 (s, 1H), 7.13 (s, 1H), 5.17 (q, J=6.0 Hz, 1H), 3.05-2.83 (m, 1H), 2.82-2.59 (m, 1H), 2.57-2.42 (m, 1H). 2.39 (s, 3H), 2.02-1.84 (m, 1H). F

### Step 2

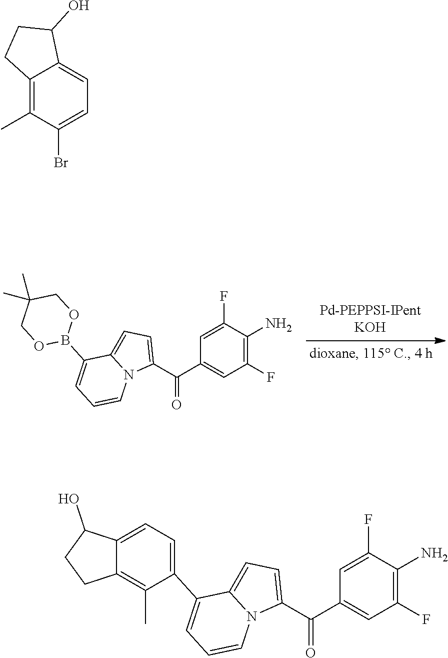 [SMILES: Cc1c(Br)ccc2c1CCC2O ; CC1(C)COB(c2cccn3c(C(=O)c4cc(F)c(N)c(F)c4)ccc23)OC1 ; Cc1c(-c2cccn3c(C(=O)c4cc(F)c(N)c(F)c4)ccc23)ccc2c1CCC2O]

A vial was loaded with 5-bromo-4-methyl-2,3-dihydro-1H-inden-1-ol (42.6 mg, 0.188 mmol). (4-amino-3,5-difluorophenyl)(8-(5,5-dimethyl-1,3,2-dioxaborinan-2-yl)indolizin-3-yl)methanone (144 mg, 0.375 mmol), ground potassium hydroxide (31.6 mg, 0.563 mmol) and dichloro[1,3-bis(2,6-Di-3-pentylphenyl)imidazol-2-ylidene](3-chloropyridyl)palladium(II) (4.5 mg, 5.6 μmol). The mixture was dissolved in dioxane (2 mL). The reaction was heated for 4 hours at 115° C. The vial was removed from heat and diluted with water and DCM. The layers were separated, and the aqueous layer was further extracted with DCM (2×10 mL). The combined organic layers were dried over Na₂SO₄ and concentrated under reduced pressure. The crude oil was loaded directly onto a 12 g silica gel column, eluting with a gradient of DCM and MeOH to obtain (4-amino-3,5-difluorophenyl)(8-(1-hydroxy-4-methyl-2,3-dihydro-1H-inden-5-yl)indolizin-3-yl)methanone.

### Step 3

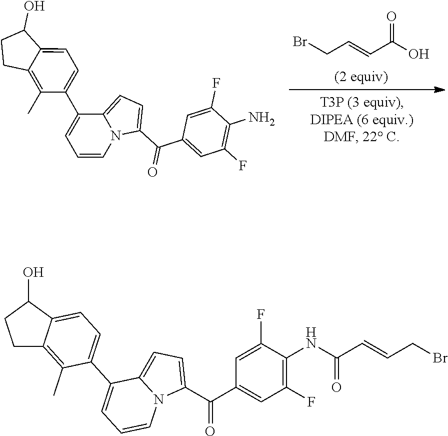 [SMILES: Cc1c(-c2cccn3c(C(=O)c4cc(F)c(N)c(F)c4)ccc23)ccc2c1CCC2O ; O=C(O)/C=C/CBr ; Cc1c(-c2cccn3c(C(=O)c4cc(F)c(NC(=O)/C=C/CBr)c(F)c4)ccc23)ccc2c1CCC2O]

A vial was loaded with (E)-4-bromobut-2-enoic acid (0.062 g. 0.376 mmol) and dissolved in 1 mL of DMF, and 2,4,6-tripropyl-1,3,5,2,4,6-trioxatriphosphinane 2,4,6-trioxide (336 μL. 0.564 mmol) was added via syringe as a 50% solution in ethyl acetate. The solution was added over another solution of (4-amino-3,5-difluorophenyl)(8-(1-hydroxy-4-methyl-2,3-dihydro-1H-inden-5-yl)indolizin-3-yl)methanone (0.079g. 0.188 mmol) in 0.5 mL of DMF. The mixture was stirred for 20 minutes, and DIPEA (0.197 mL. 1.128 mmol) was added via syringe. The mixture was stirred for 3 hours. The mixture was diluted with 10 mL of EtOAc, and 4 mL of water was added. The aqueous solution was extracted with EtOAc (3×10 mL), and the organic layers combined were washed with brine (2×5 mL). The solvent was evaporated and the crude mixture was taken to the next step without further purification. (E)-4-bromo-N-(2,6-difluoro-4-(8-(1-hydroxy-4-methyl-2,3-dihydro-1H-inden-5-yl)indolizine-3-carbonyl)phenyl)but-2-enamide were obtained.

### Step 4

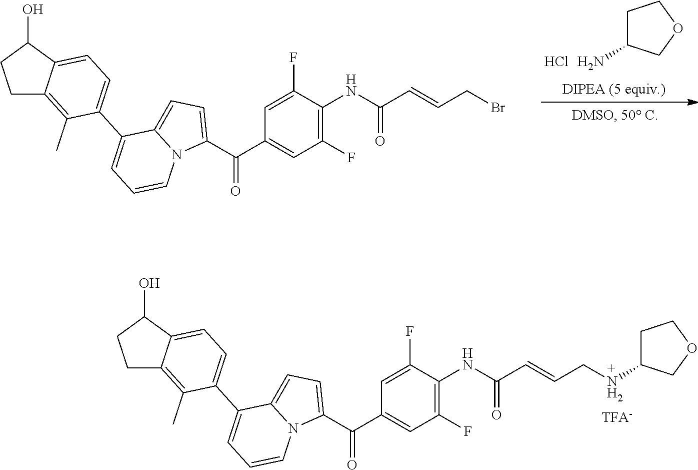 [SMILES: Cc1c(-c2cccn3c(C(=O)c4cc(F)c(NC(=O)/C=C/CBr)c(F)c4)ccc23)ccc2c1CCC2O ; Cl ; N[C@@H]1CCOC1 ; Cc1c(-c2cccn3c(C(=O)c4cc(F)c(NC(=O)/C=C/CN[C@@H]5CCOC5)c(F)c4)ccc23)ccc2c1CCC2O]

A vial was loaded with a crude mixture containing (E)-4-bromo-N-(2,6-difluoro-4-(8-(1-hydroxy-4-methyl-2,3-dihydro-1H-inden-5-yl)indolizine-3-carbonyl)phenyl)but-2-enamide. (S)-tetrahydrofuran-3-amine hydrochloride (88 mg, 0.712), 1 mL of DMSO and DIPEA (197 μL, 1.128 mmol). The mixture was stirred for 3 hours at 50° C. The mixture was filtered and submitted to HPLC purification under reverse phase. (3S)—N-((E)-4-((2,6-difluoro-4-(8-(1-hydroxy-4-methyl-2,3-dihydro-1H-inden-5-yl)indolizine-3-carbonyl)phenyl)amino)-4-oxobut-2-en-1-yl)tetrahydrofuran-3-aminium 2,2,2-trifluoroacetate was obtained (Ex. 132). MS (ESI): [M+H]⁺ m/z: 572, 1H NMR (499 MHz, DMSO) δ 10.35 (s, 1H), 9.87 (d, J=7.3 Hz, 1H), 9.09 (s, 2H), 7.59 (d, J=7.9 Hz, 2H), 7.44 (d, J=4.3 Hz, 1H), 7.26 (dd, J=15.8, 7.5 Hz, 3H), 6.82 (dt, J=13.5, 6.6 Hz, 1H), 6.54 (d, J=15.4 Hz, 1H), 6.22 (d, J=4.7 Hz, 1H), 5.09 (s, 1H), 3.91 (d, J=6.6 Hz, 5H). 3.78 (dd, J=11.0, 6.4 Hz, 2H), 3.73-3.62 (m, 2H), 2.96 (d, J=8.9 Hz, 1H), 2.77 (dt, J=15.9, 7.9 Hz, 1H), 2.38 (d, J=7.3 Hz, 1H), 2.26 (dq, J=14.2, 7.9 Hz, 1H), 2.10 (s, 3H), 2.02 (s, 1H), 1.84 (s, 1H).

### Test Example 1: KRASG12C Binding Assay

As test compounds for the KRASG12C binding assay, the compounds obtained in the “Preparation of Compounds” section were used. Sample solutions each containing a test compound were prepared in the form of a 10 mM DMSO solution.

Gppcp-bound recombinant K-Ras4B G12C mutant protein (amino acids 1-169, C51S/C80L/C118S, SEQ ID NO:1) was diluted with a buffer (1×TBS, 0.1 mM TCEP, 0.25 mM MgCl₂, 50 μM Gppcp) to prepare a 50 nM protein solution. K-Ras4B is the major splicing variant of K-Ras. Amino acid sequence of residues 1 to 150 including the compounds' binding region of K-Ras4B is exactly same as that of K-Ras4A, the other splicing variant (Oncotarget. 2016 Jul. 19; 7(29): 46717-46733). A sample solution containing 10 mM test compound was diluted ten-fold with DMSO to make a 1 mM sample solution, and then diluted twenty-fold with DMSO to make a 50 μM sample solution. 1 μL of a 50 μM sample solution was added to 100 μL of a 50 nM protein solution to adjust the final concentration of a test compound to be 0.5 μM in the mixture. The mixtures were stored in an incubator at 25° C. for 1 hour, and 10 μL of a 1×TBS solution containing 2% formic acid was added to stop the reaction, followed by LC-MS measurement. LC-MS measurement was performed using Xevo G2-S Q-Tof manufactured by Waters, and reverse-phase chromatography was performed with a desalting column. A mass spectrum of positive ions was obtained by electrospray. For a mass spectrum, a spectrum of polyvalent ions was collectively converted to a molecular weight by using OpenLynx software by the MaxEnt technique, and a compound binding rate was calculated from the ratio of the signal intensity of a peak that corresponds to the molecular weight of the protein to the signal intensity of a peak that corresponds to the molecular weight of the protein conjugated with the compound.

The binding assay of the test compounds was conducted at a final concentration of 0.5 μM. A binding rate of 80% or more is rated “A”, a binding rate of 60% or more and less than 80% is rated “B”, a binding rate of 40% or more and less than 60% is rated “C”, a binding rate of 20% or more and less than 40% is rated “D”, and a binding rate of less than 20% is rated “E”. N.D.=not determined. Binding rate % is the ratio of KRAS signal intensity at the molecular weight of one molecule adduct to the sum of all the KRAS signal intensity (unbound form and adducts) in mass spectrum. Thus, binding rate 100% means that only the one molecule adduct was observed without any unbound KRAS. The following tables show the results.

TABLE 1

| Example | |
|---|---|
| 1 | A |
| 2 | A |
| 3 | A |
| 4 | A |
| 5 | A |
| 6 | B |
| 7 | B |
| 8 | A |
| 9 | A |
| 10 | A |
| 11 | A |
| 12-1 | A |
| 12-2 | B |
| 13-1 | A |
| 13-2 | A |
| 14-1 | A |
| 14-2 | A |
| 15-1 | A |
| 15-2 | A |
| 16 | A |
| 17 | A |
| 18-1 | A |
| 18-2 | A |
| 19-1 | A |
| 19-2 | A |
| 20-1 | A |
| 20-2 | A |
| 21 | A |
| 22-1 | A |
| 22-2 | A |
| 23 | A |
| 24 | A |
| 25 | A |
| 26 | A |
| 27-1 | A |
| 27-2 | A |
| 28-1 | A |
| 28-2 | A |
| 29 | A |
| 30-1 | A |
| 30-2 | A |
| 31 | A |
| 32-1 | A |
| 32-2 | A |
| 33 | A |
| 34-1 | A |
| 34-2 | B |
| 35-1 | A |
| 35-2 | A |
| 36-1 | B |
| 36-2 | A |
| 37 | A |
| 38 | A |
| 39-1 | A |
| 39-2 | A |
| 40-1 | A |
| 40-2 | A |
| 41-1 | A |
| 41-2 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56-1 | A |
| 56-2 | A |
| 57-1 | A |
| 57-2 | A |
| 58 | A |
| 59 | A |
| 60-1 | A |
| 60-2 | A |
| 61-1 | B |
| 61-2 | A |
| 62 | A |
| 63 | A |
| 64 | A |
| 65 | A |
| 66 | A |
| 67 | A |
| 68 | A |
| 69 | A |
| 70 | A |
| 71-1 | A |
| 71-2 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83-1 | A |
| 83-2 | B |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |

TABLE 1.2

| Example | |
|---|---|
| 102-1 | A |
| 102-2 | C |
| 103 | A |
| 104 | A |
| 105-1 | A |
| 105-2 | C |
| 106 | A |
| 107 | A |
| 108 | A |
| 109-1 | A |
| 109-2 | D |
| 110 | A |
| 111-1 | A |
| 111-2 | A |
| 112-1 | N.D. |
| 112-2 | A |
| 113 | A |
| 114 | A |
| 115 | B |

### Test Example 2: Evaluation of Inhibitory Activity of Compounds on Interaction Between KRAS G12C and cRAF (In Vitro)

Interaction between recombinant K-Ras4B G12C mutant protein (amino acids 1-169, SEQ ID NO:2) and cRAF RAS-binding domain (cRAF-RBD, amino acids 50-132, Jena Biosciences GmbH) was measured using Alpha technology (PerkinElmer Inc.).

Recombinant KRAS G12C protein with an N-terminal His-tag was expressed in E. coli and purified by affinity chromatography. To prepare GTP-bound form and GDP-bound form of KRAS G12C protein. 50 μM KRAS G12C protein was incubated with 1 mM GMPPNP (Guanosine-5′-[(β,γ)-imido]triphosphate. Tetralithium salt)(Jena Bioscience GmbH) and 1 mM GDP, respectively, in a loading buffer (20 mM Tris-HCl (pH 7.5), 50 mM NaCl, 1 mM DTT and 2.5 mM EDTA) for 1 hour on ice. After the incubation, MgCl2 was added to a final concentration of 10 mM, followed by incubation at room temperature for 30 minutes. The mixture was allowed to pass through a NAP-5 column to remove free nucleotides and purified, and the resultant nucleotide-bound KRAS G12C protein was used for compound evaluation.

For the measurement of the inhibitory activity of compounds on interaction between KRAS G12C and cRAF-RBD, GMPPNP-bound KRAS G12C protein was incubated with various concentrations of compound in a reaction buffer (20 mM Tris-HCl (pH 7.5), 100 mM NaCl, 1 mM MgCl₂, 1 mM TCEP. 0.1% Tween 20) at 25° C. for 1 hour. After the incubation, recombinant cRAF-RBD and Alpha detection reagents were added and incubated at room temperature for 1.5 hours for binding. Interaction of KRAS G12C and cRAF-RBD was monitored by measuring Alpha signal. Inhibition % was calculated using the signal from the reaction without test compound (DMSO control) as 0% inhibition and the signal from the reaction using GDP-bound KRAS G12C in place of GMPPNP-bound KRAS G12C as 100% inhibition. IC50 values were calculated from dose titration curve using curve fitting by XLfit software (IDBS). The following tables show the results.

TABLE 2

| Example | IC50 (μM) |
|---|---|
| 1 | 0.058 |
| 2 | 0.060 |
| 3 | 0.072 |
| 4 | 0.074 |
| 5 | 0.075 |
| 6 | 0.070 |
| 7 | 0.069 |
| 8 | 0.089 |
| 9 | 0.027 |
| 10 | 0.069 |
| 11 | 0.021 |
| 12-1 | 0.029 |
| 12-2 | 0.16 |
| 13-1 | 0.046 |
| 13-2 | 0.32 |
| 14-1 | 0.057 |
| 14-2 | 0.056 |
| 15-1 | 0.042 |
| 15-2 | 0.19 |
| 16 | 0.064 |
| 17 | 0.048 |
| 18-1 | 0.029 |
| 18-2 | 0.22 |
| 19-1 | 0.020 |
| 19-2 | 0.14 |
| 20-1 | 0.20 |
| 20-2 | 0.033 |
| 21 | 0.097 |
| 22-1 | 0.023 |
| 22-2 | 0.11 |
| 23 | 0.023 |
| 24 | 0.054 |
| 25 | 0.079 |
| 26 | 0.062 |
| 27-1 | 0.025 |
| 27-2 | 0.12 |
| 28-1 | 0.031 |
| 28-2 | 0.80 |
| 29 | 0.090 |
| 30-1 | 0.13 |
| 30-2 | 0.061 |
| 31 | 0.062 |
| 32-1 | 0.021 |
| 32-2 | 0.42 |
| 33 | 0.075 |
| 34-1 | 0.094 |
| 34-2 | 0.20 |
| 35-1 | 0.029 |
| 35-2 | 0.053 |
| 36-1 | 0.040 |
| 36-2 | 0.056 |
| 37 | 0.087 |
| 38 | 0.077 |
| 39-1 | 0.58 |
| 39-2 | 0.032 |
| 40-1 | 0.34 |
| 40-2 | 0.021 |
| 41-1 | 0.052 |
| 41-2 | 0.074 |
| 42 | 0.085 |
| 43 | 0.088 |
| 44 | 0.022 |
| 45 | 0.076 |
| 46 | 0.030 |
| 47 | 0.016 |
| 48 | 0.11 |
| 49 | 0.019 |
| 50 | 0.25 |
| 51 | 0.057 |
| 52 | 0.075 |
| 53 | 0.040 |
| 54 | 0.067 |
| 55 | 0.041 |
| 56-1 | 0.45 |
| 56-2 | 0.021 |
| 57-1 | 0.043 |
| 57-2 | 0.79 |
| 58 | 0.035 |
| 59 | 0.034 |
| 60-1 | 1.6 |
| 60-2 | 0.026 |
| 61-1 | 1.9 |
| 61-2 | 0.028 |
| 62 | 0.066 |
| 63 | 0.071 |
| 64 | 0.046 |
| 65 | 0.076 |
| 66 | 0.070 |
| 67 | 0.038 |
| 68 | 0.077 |
| 69 | 0.087 |
| 70 | 0.070 |
| 71-1 | 0.043 |
| 71-2 | 0.22 |
| 72 | 0.089 |
| 73 | 0.073 |
| 74 | 0.064 |
| 75 | 0.037 |
| 76 | 0.075 |
| 77 | 0.042 |
| 78 | 0.051 |
| 79 | 0.061 |
| 80 | 0.059 |
| 81 | 0.070 |
| 82 | 0.096 |
| 83-1 | 0.016 |
| 83-2 | 0.92 |
| 84 | 0.091 |
| 85 | 0.045 |
| 86 | 0.040 |
| 87 | 0.035 |
| 88 | 0.026 |
| 89 | 0.022 |
| 90 | 0.097 |
| 91 | 0.075 |
| 92 | 0.080 |
| 93 | 0.025 |
| 94 | 0.083 |
| 95 | 0.13 |
| 96 | 0.036 |
| 97 | 0.044 |
| 98 | 0.041 |
| 99 | 0.17 |
| 100 | 0.071 |
| 101 | 0.054 |

TABLE 2.2

| Example | IC50 (μM) |
|---|---|
| 102-1 | 0.05716 |
| 102-2 | 2.4384 |
| 103 | 0.03197 |
| 104 | 0.0789 |
| 105-1 | 0.0298 |
| 105-2 | 1.44974 |
| 106 | 0.0311 |
| 107 | 0.03763 |
| 108 | 0.04046 |
| 109-1 | 0.01919 |
| 109-2 | 1.206 |
| 110 | 0.08074 |
| 111-1 | 0.00871 |
| 111-2 | 0.01739 |
| 112-1 | 0.02017 |
| 112-2 | 0.36208 |
| 113 | 0.0449 |
| 114 | 0.01894 |
| 115 | 0.45245 |

### Test Example 3: Assay of Growth Inhibition Activity on KRAS-G12C Mutant Cell Line (MIA PaCa-2) (In Vitro)

MIA PaCa-2 cells (given by Sumitomo Dainippon Pharma Co., Ltd.), which are a KRAS-G12C mutant human pancreas cancer cell line, were suspended in a 10% fetal bovine serum-containing RPMI1640 medium (manufactured by Fujifilm Wako Pure Chemical Corporation.). The cell suspension was seeded into each well of a 384-well U bottom microplate and cultured in an incubator containing 5% CO2 gas at 37° C. for 1 day. The compounds obtained in the “Preparation of Compounds” section were used as test compounds and were dissolved in DMSO, respectively, and each test compound was diluted with DMSO to give a concentration 500 times the final concentration. The resultant solution of the test compound in DMSO was diluted with the medium used for suspending cells and added to each well of the cell-culture plate to give a DMSO final concentration of 0.2%, followed by culture in an incubator containing 5% CO2 gas at 37° C. for another 3 days. The cell count after 3-day culture in the presence of the test compound was measured using CellTiter-Glo 3D Reagent (manufactured by Promega Corporation). All wells were added with CellTiter-Glo 3D Reagent and mixed for 10 minutes. 30 minutes after mixing, luminescence was measured by a plate reader. The growth inhibition rate was calculated from the following equation, and the concentration of the test compound at which 50/o inhibition was achieved (IC50 (nM)) was determined. The following tables show the results.

$$Growth Inhibition Rate ( \% ) = ( C - T ) / ( C ) \times 100$$

T: the emission intensity in a well into which a test compound was added.
C: the emission intensity in a well into which a test compound was not added.

TABLE 3

| Example | IC50 (μM) |
|---|---|
| 1 | 0.091 |
| 2 | 0.057 |
| 3 | 0.052 |
| 4 | 0.042 |
| 5 | 0.045 |
| 6 | 0.094 |
| 7 | 0.16 |
| 8 | 0.13 |
| 9 | 0.082 |
| 10 | 0.094 |
| 11 | 0.068 |
| 12-1 | 0.079 |
| 12-2 | 0.27 |
| 13-1 | 0.032 |
| 13-2 | 0.22 |
| 14-1 | 0.028 |
| 14-2 | 0.042 |
| 15-1 | 0.046 |
| 15-2 | 0.22 |
| 16 | 0.048 |
| 17 | 0.032 |
| 18-1 | 0.029 |
| 18-2 | 0.23 |
| 19-1 | 0.047 |
| 19-2 | 0.16 |
| 20-1 | 0.20 |
| 20-2 | 0.059 |
| 21 | 0.18 |
| 22-1 | 0.072 |
| 22-2 | 0.41 |
| 23 | 0.52 |
| 24 | 0.054 |
| 25 | 0.10 |
| 26 | 0.082 |
| 27-1 | 0.12 |
| 27-2 | 0.53 |
| 28-1 | 0.051 |
| 28-2 | 0.94 |
| 29 | 0.12 |
| 30-1 | 0.36 |
| 30-2 | 0.12 |
| 31 | 0.064 |
| 32-1 | 0.014 |
| 32-2 | 0.99 |
| 33 | 0.14 |
| 34-1 | 0.13 |
| 34-2 | 0.31 |
| 35-1 | 0.035 |
| 35-2 | 0.073 |
| 36-1 | 0.024 |
| 36-2 | 0.060 |
| 37 | 0.24 |
| 38 | 0.078 |
| 39-1 | 0.35 |
| 39-2 | 0.044 |
| 40-1 | 0.34 |
| 40-2 | 0.073 |
| 41-1 | 0.032 |
| 41-2 | 0.050 |
| 42 | 0.61 |
| 43 | 0.16 |
| 44 | 0.023 |
| 45 | 0.067 |
| 46 | 0.024 |
| 47 | 0.075 |
| 48 | 0.23 |
| 49 | 0.049 |
| 50 | 0.22 |
| 51 | 0.12 |
| 52 | 0.19 |
| 53 | 0.022 |
| 54 | 0.13 |
| 55 | 0.088 |
| 56-1 | 1.1 |
| 56-2 | 0.029 |
| 57-1 | 0.020 |
| 57-2 | 1.1 |
| 58 | 0.069 |
| 59 | 0.23 |
| 60-1 | 1.6 |
| 60-2 | 0.015 |
| 61-1 | 2.3 |
| 61-2 | 0.010 |
| 62 | 0.14 |
| 63 | 0.40 |
| 64 | 0.025 |
| 65 | 0.19 |
| 66 | 0.097 |
| 67 | 0.051 |
| 68 | 0.067 |
| 69 | 0.34 |
| 70 | 0.21 |
| 71-1 | 0.13 |
| 71-2 | 0.57 |
| 72 | 0.10 |
| 73 | 0.30 |
| 74 | 0.065 |
| 75 | 0.23 |
| 76 | 0.30 |
| 77 | 0.071 |
| 78 | 0.12 |
| 79 | 0.11 |
| 80 | 0.085 |
| 81 | 0.071 |
| 82 | 0.26 |
| 83-1 | 0.016 |
| 83-2 | 0.85 |
| 84 | 0.12 |
| 85 | 0.11 |
| 86 | 0.081 |
| 87 | 0.14 |
| 88 | 0.13 |
| 89 | 0.10 |
| 90 | 0.20 |
| 91 | 0.10 |
| 92 | 0.12 |
| 93 | 0.13 |
| 94 | 0.28 |
| 95 | 0.28 |
| 96 | 0.13 |
| 97 | 0.32 |
| 98 | 0.042 |
| 99 | 0.17 |
| 100 | 0.096 |
| 101 | 0.093 |

TABLE 3.2

| Example | IC50 (μM) |
|---|---|
| 102-1 | 0.02014 |
| 102-2 | 1.46224 |
| 103 | 0.07481 |
| 104 | 0.09042 |
| 105-1 | 0.02313 |
| 105-2 | 0.94317 |
| 106 | 0.13434 |
| 107 | 0.56937 |
| 108 | 0.07769 |
| 109-1 | 0.02156 |
| 109-2 | 0.78449 |
| 110 | 0.18204 |
| 111-1 | 0.04647 |
| 111-2 | 0.19116 |
| 112-1 | 0.04261 |
| 112-2 | 0.53216 |
| 113 | 0.0559 |
| 114 | 1.07337 |
| 115 | 0.24844 |

### Test Example 4: Procedure for RAS:RAF-RBD Binding Assay

The RAF-Ras binding domain (RBD) protein interaction assay utilizes recombinant biotinylated KRAS protein containing a G12C mutation (SEQ ID NO:3) and the GST-tagged Ras binding domain of c-RAF (residues 50-132) from Jena Biosciences (catalog #PR-366). It should be noted that KRAS-G12C protein is pre-loaded with a GTP analog. GMP-PNP to mimic KRAS in the activated state. Compounds are added to KRAS and then after a 30-minute incubation time, the RAF-RBD and detection antibodies are added. Small molecule inhibitors that block the interaction of c-RAF-RBD prevent generation of a TR-FRET signal.

Biotinylated KRAS G12C protein is diluted to 20 nM in assay buffer (20 mM HEPES pH 7.5, 150 mM sodium chloride, 10 mM magnesium chloride, and 0.01% Tween20).

Each test compound (10 mM stock in DMSO) is diluted in DMSO to make a 10-point, 3-fold dilution series in a 384-well low dead volume microplate (Labcyte, catalog #LP-0200). Once titrations are made, 50 nL of the diluted compounds is acoustically dispensed into 384-well plates (Corning, catalog #3820) using an Echo 655 (Labcyte).

Each well of the assay plate receives 5 μL of Biotinylated KRAS G12C assay solution and is incubated at room temperature for 30 minutes. Each well then receives 5 μL of 100 nM GST-c-RAF RBD protein and a 1:100 dilution of both anti-GST-d2 (Cisbio catalog #61GSTDLA) and Strepavidin-Tb cryptate (Cisbio catalog #610SATLA) in assay buffer, and the plate is mixed and briefly centrifuged followed by a 60 minute incubation at room temperature.

The time-resolved fluorescence resonance energy transfer signal of both plates is measured on an Envision (PerkinElmer) plate reader: dichroic mirror=LANCE/DELFIA DUAL/Bias; Emission1=615 nm; Emission2=665 nm; delay time=60 ms. The signal of each well is determined as the ratio of the emission at 665 nm to that 615 nm. Percent effect of each well is determined after normalization to control wells containing DMSO (no effect) or a saturating concentration of inhibitor (max effect). The apparent effect as a function of compound concentration is fit to a four-parameter logistic equation. Table 4 shows the results.

TABLE 4

| Example | IC50 (μM) |
|---|---|
| 116 | 0.01483 |
| 117 | 0.05646 |
| 118 | 0.4612 |
| 119 | n/a |
| 120 | 0.4818 |
| 121 | 0.1684 |
| 122 | n/a |
| 123 | 0.07844 |
| 124 | n/a |
| 125 | 0.3984 |
| 126 | 0.0283 |
| 127 | 0.4026 |
| 128 | 0.1594 |
| 129 | 0.1009 |
| 130 | 0.09592 |
| 131a | 0.01343 |
| 131b | 0.01239 |
| 132 | 0.3003 |

### Sequences

Gppcp-bound recombinant K-Ras4B G12C mutant protein (amino acids 1-169,
C51S/C80L/C118S) (SEQ ID NO: 1):
GMTEYKLVVVGACGVGKSALTIQLIQNHFVDEYDPTIEDSYRKQVVIDGETSLLDI
LDTAGQEEYSAMRDQYMRTGEGFLLVFAINNTKSFEDIHHYREQIKRVKDSEDVP
MVLVGNKSDLPSRTVDTKQAQDLARSYGIPFIETSAKTRQGVDDAFYTLVREIRK
HKEK
Recombinant K-Ras4B G12C mutant protein (SEQ ID NO: 2):
MASSHHHHHHSSENLYFQGMTEYKLVVVGACGVGKSALTIQLIONHFVDEYDPTI
EDSYRKQVVIDGETCLLDILDTAGQEEYSAMRDQYMRTGEGFLCVFAINNTKSFE
DIHHYREQIKRVKDSEDVPMVLVGNKCDLPSRTVDTKQAQDLARSYGIPFIETSA
KTRQGVDDAFYTLVREIRKHKEK
Gppnp-bound recombinant K-Ras4B G12C mutant protein (SEQ ID NO: 3):
GLNDIFEAQKIEWHETEYKLVVVGACGVGKSALTIQLIQNHFVDEYDPTIEDSYRK
QVVIDGETSLLDILDTAGQEEYSAMRDQYMRTGEGFLLVFAINNTKSFEDIHHYRE
QIKRVKDSEDVPMVLVGNKSDLPSRTVDTKQAQDLARSYGIPFIETSAKTRQGVD
DAFYTLVREIRKHKEK

1. A compound selected from the group consisting of: 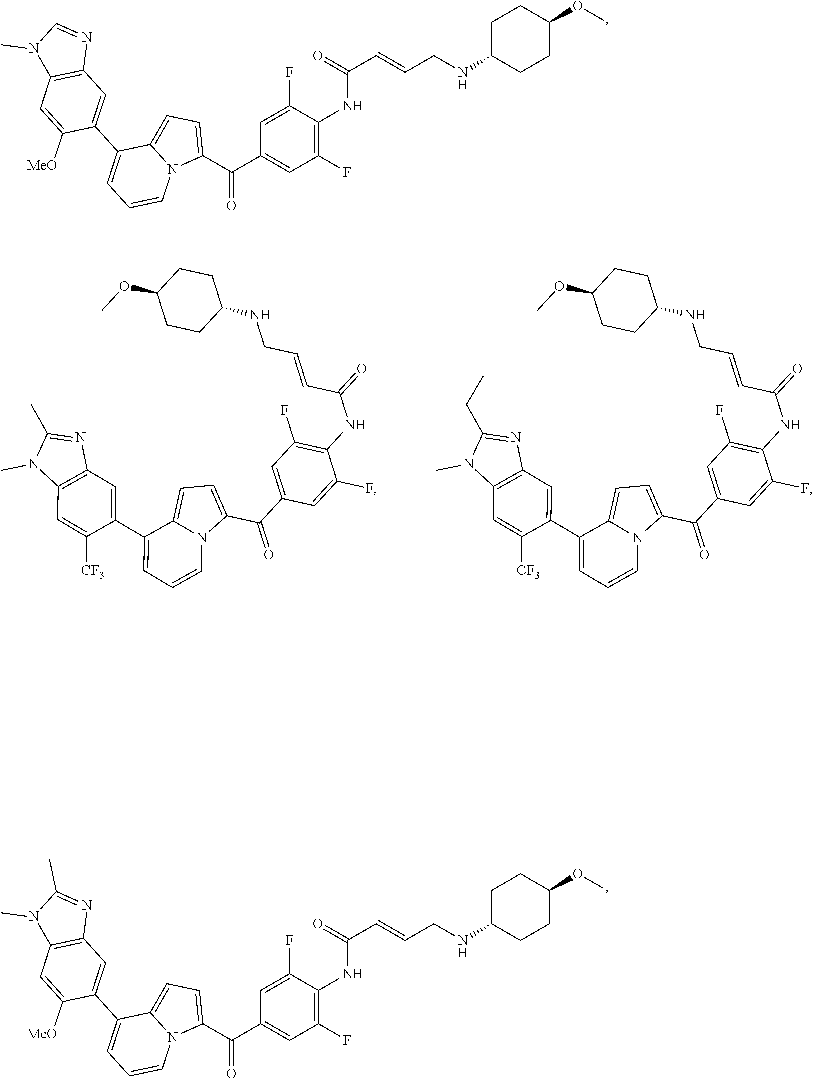 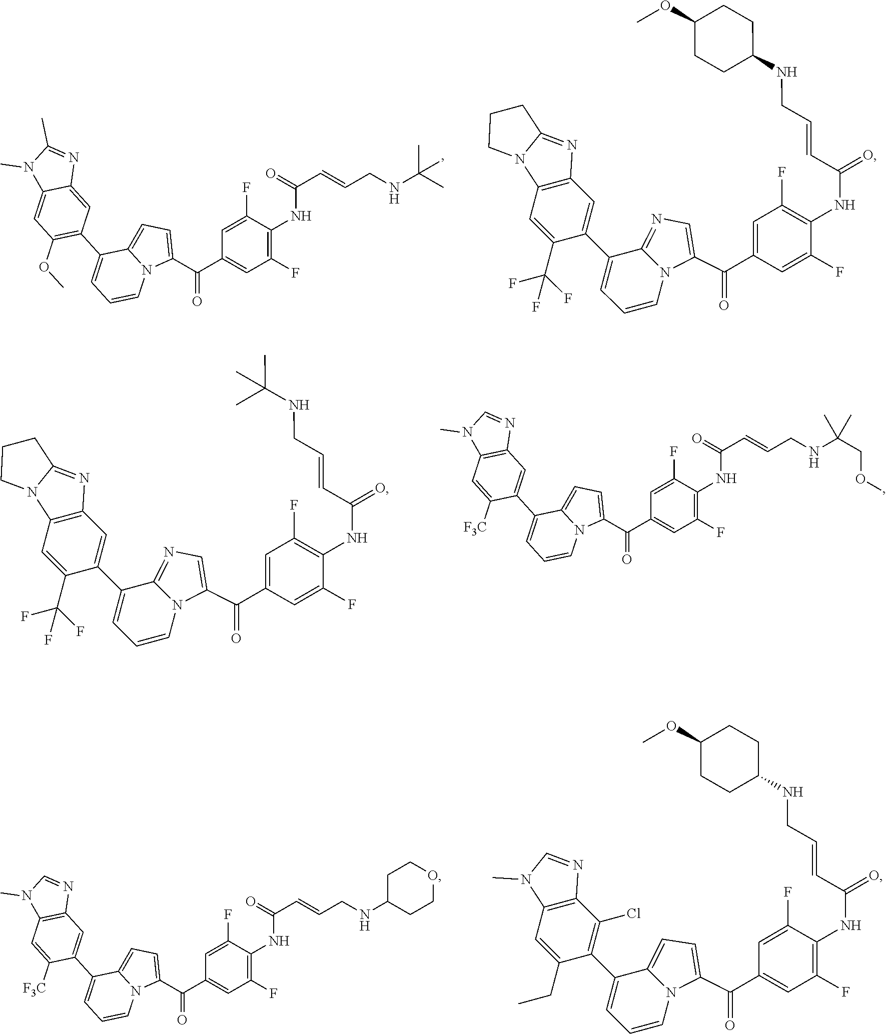 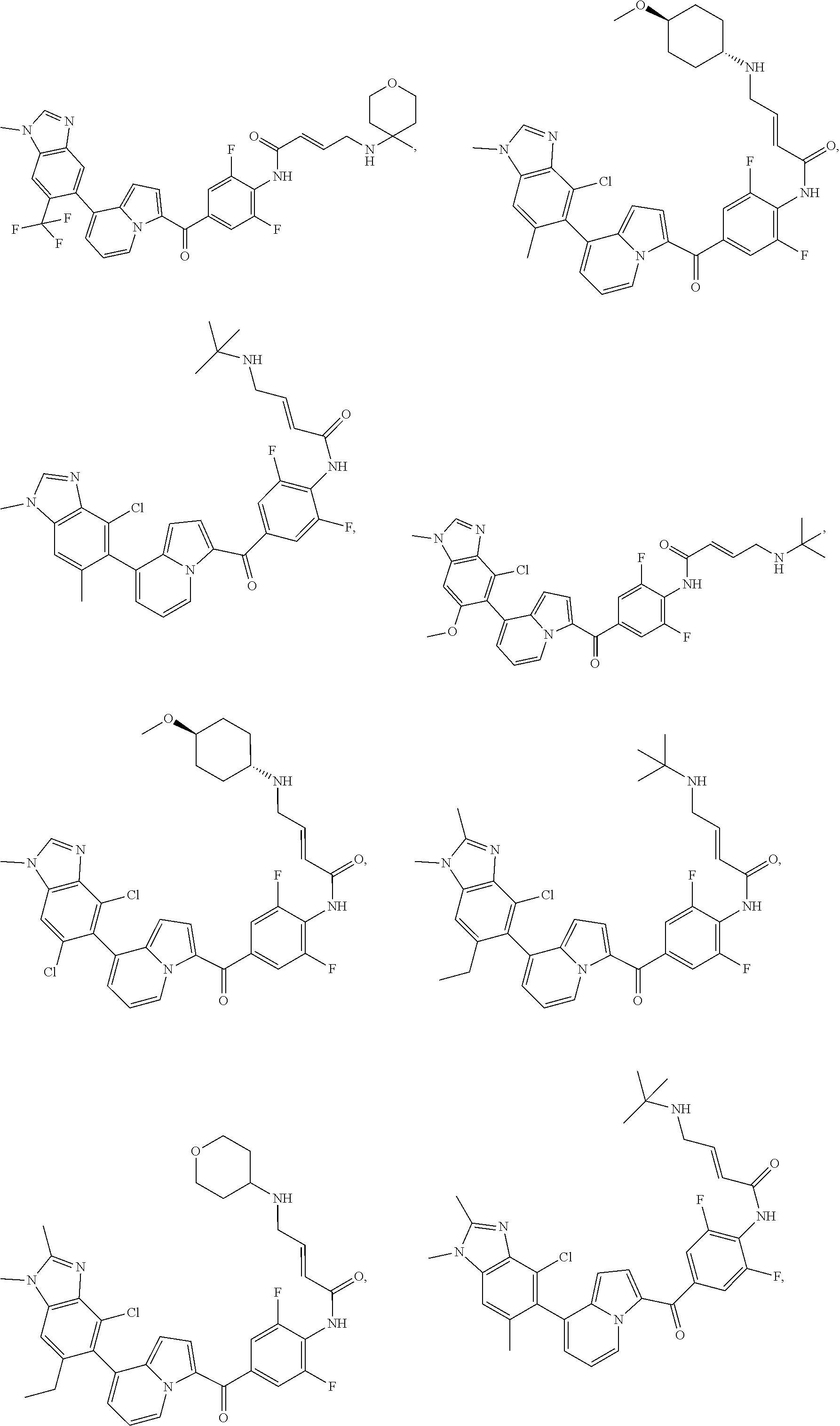 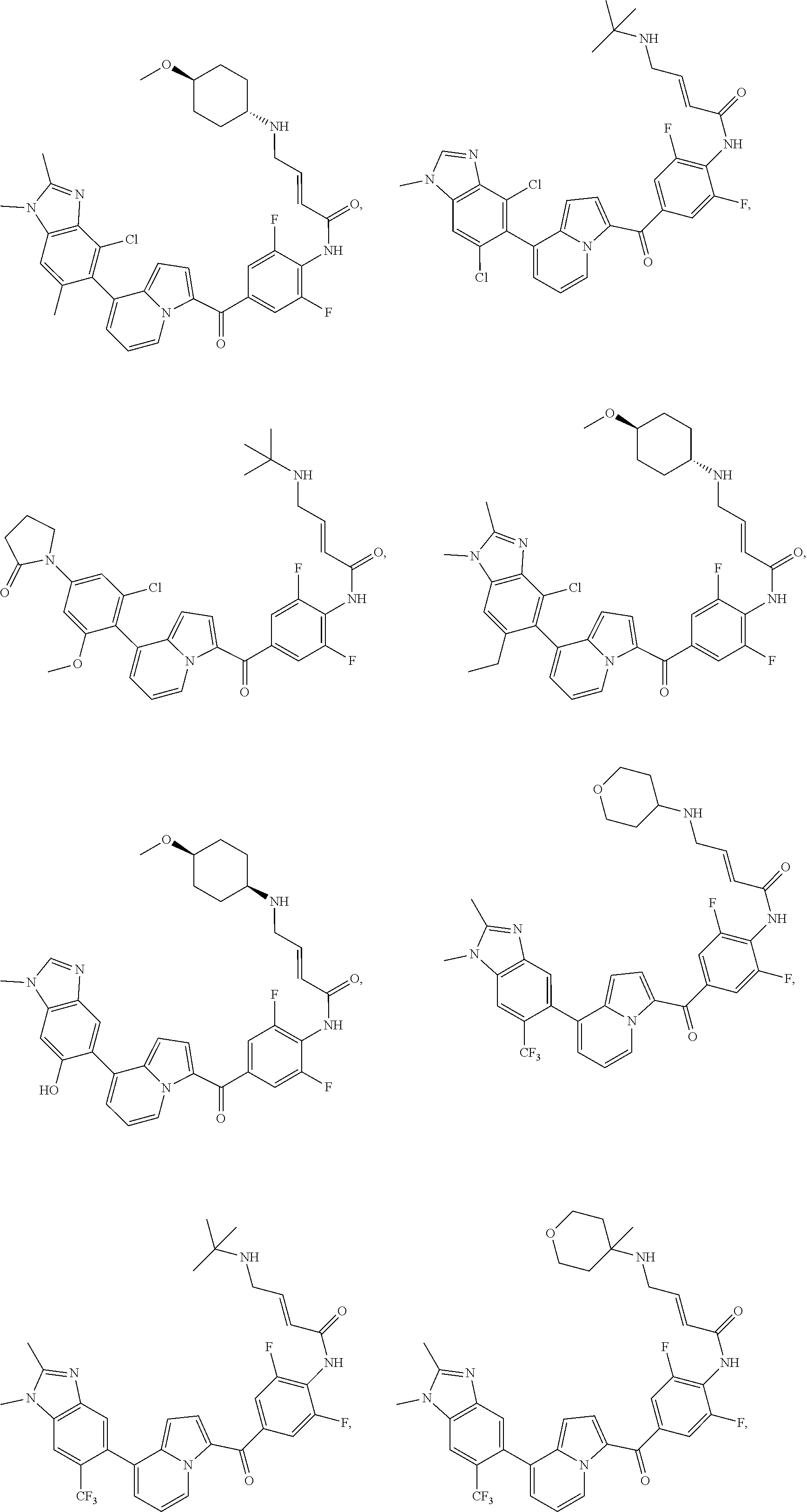 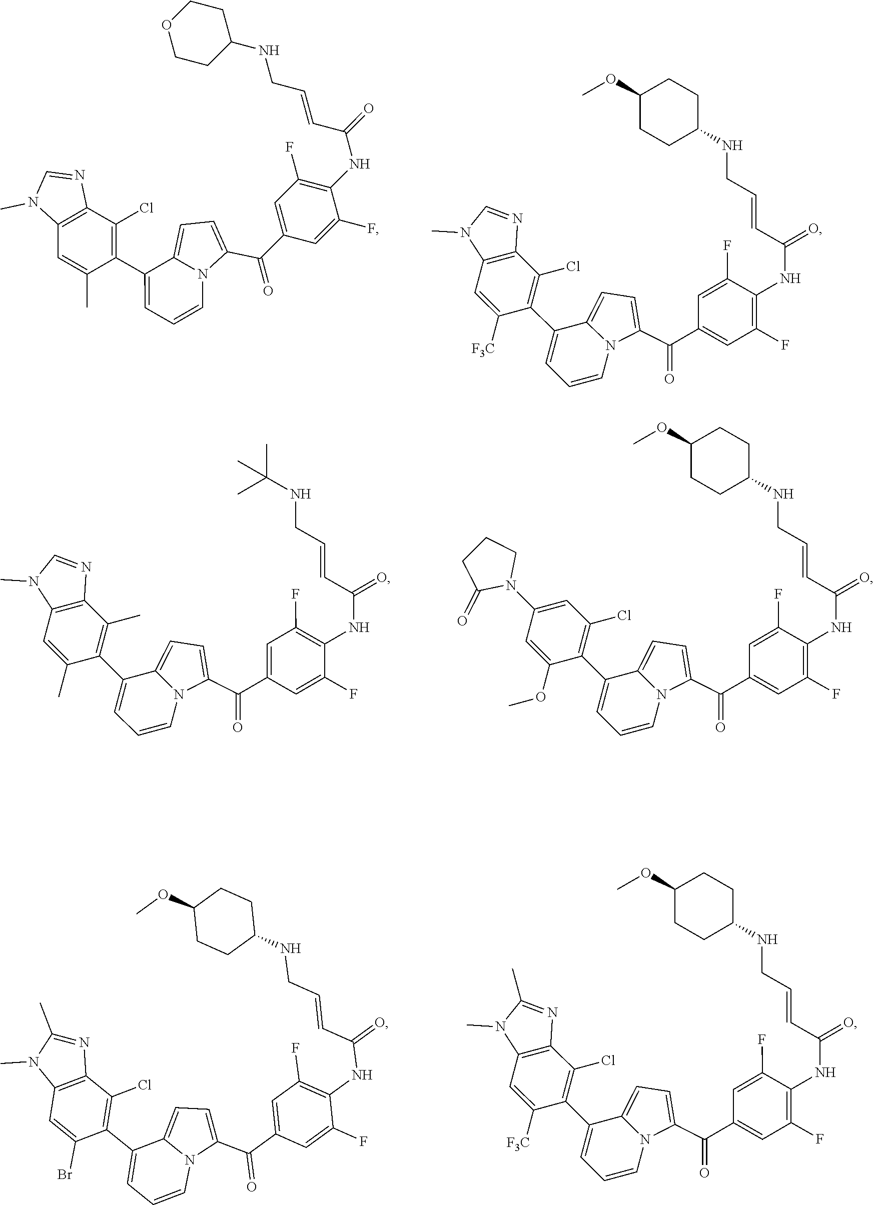 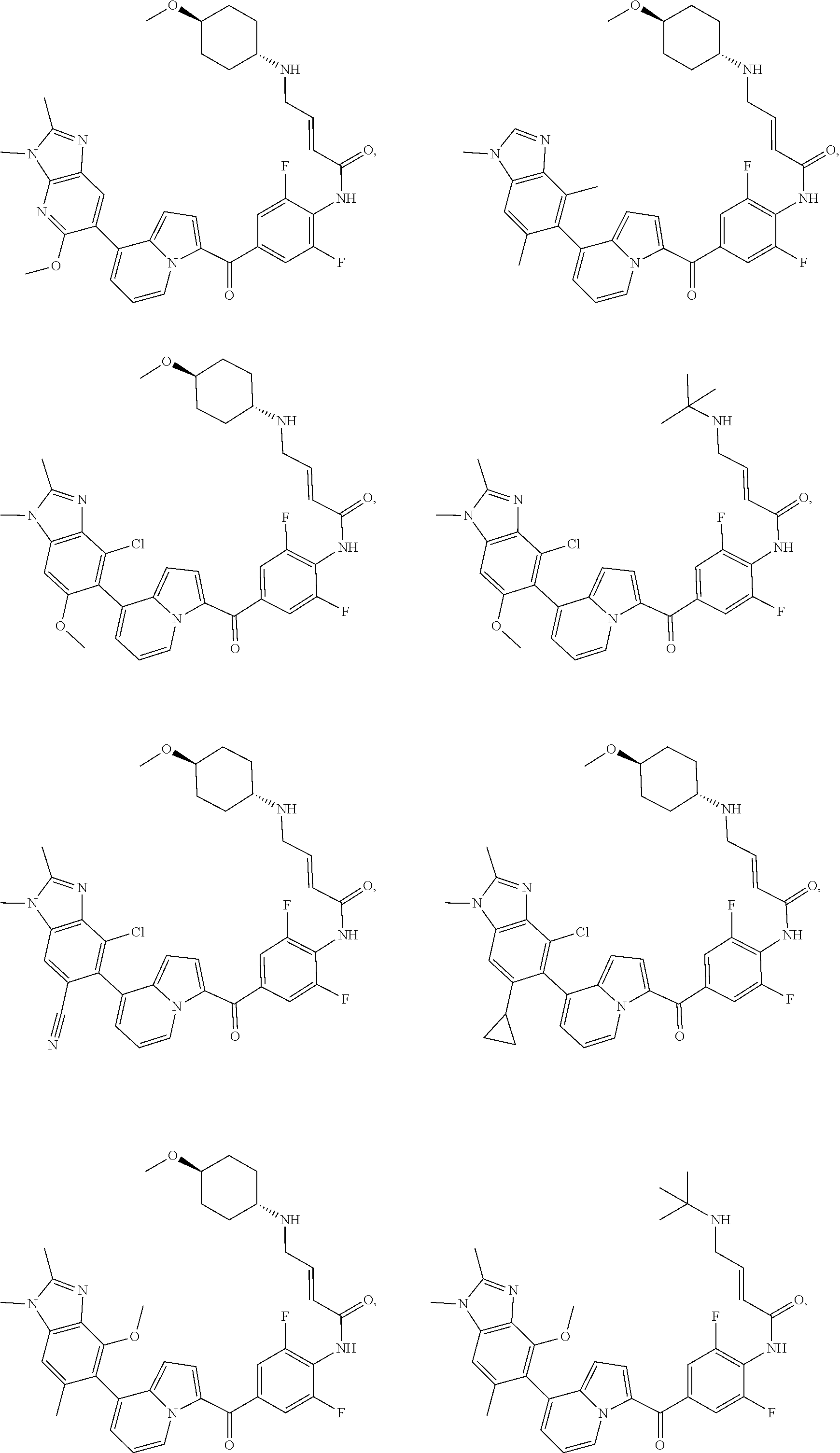 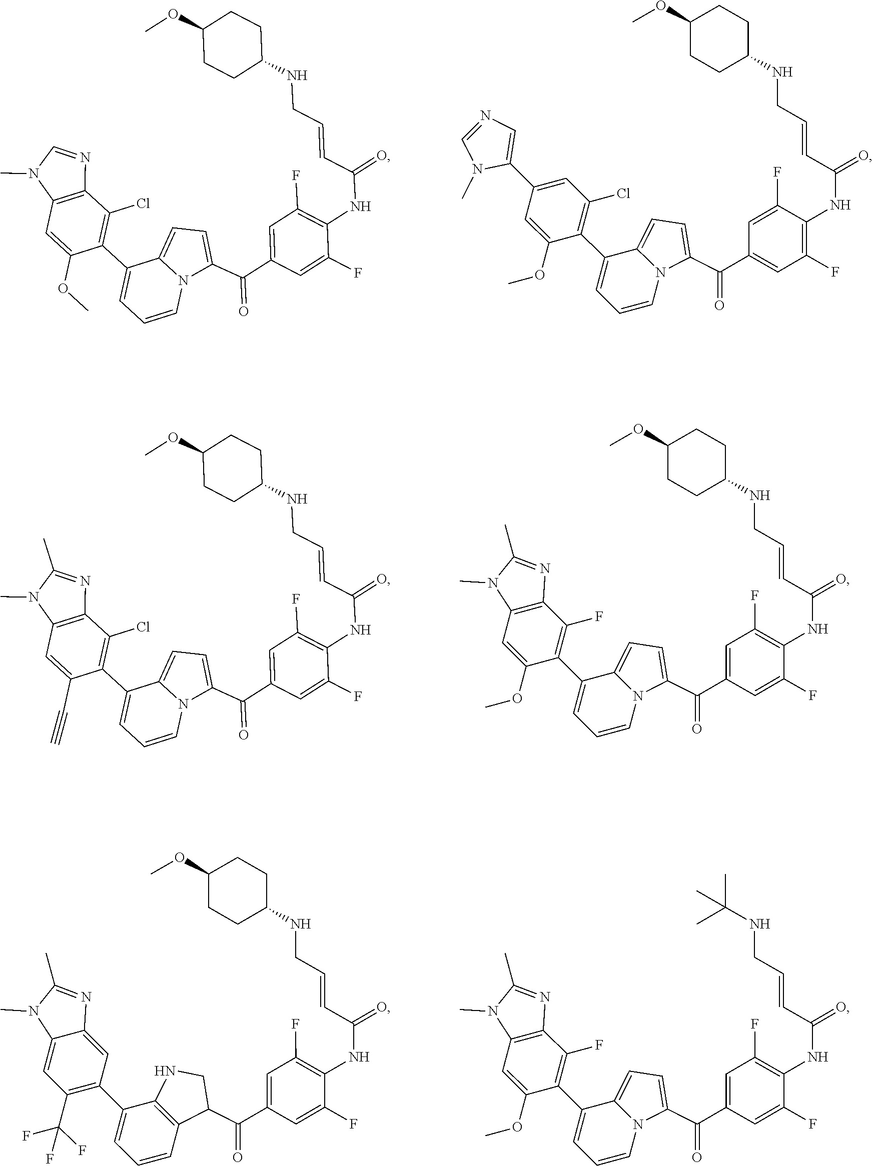 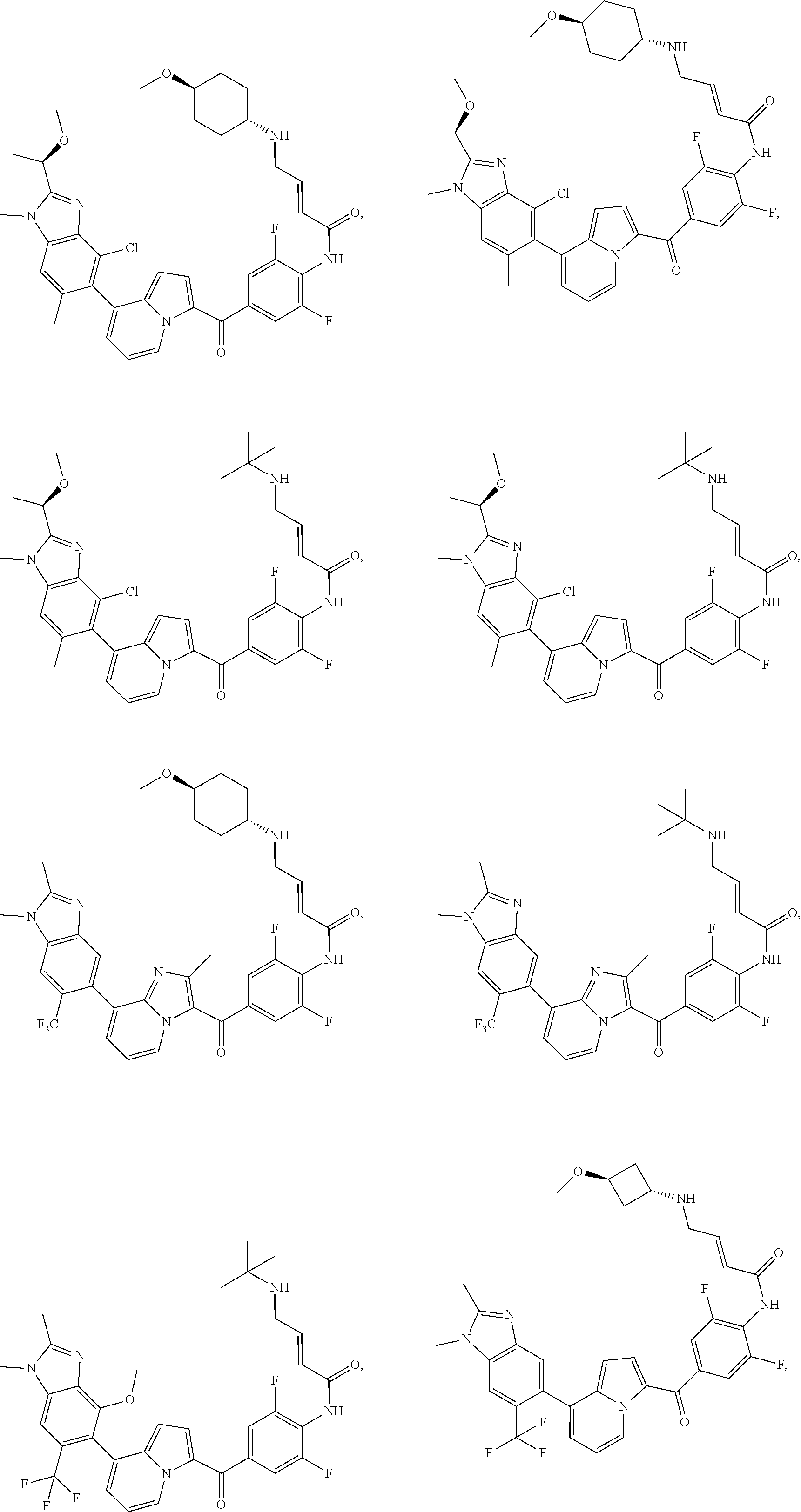 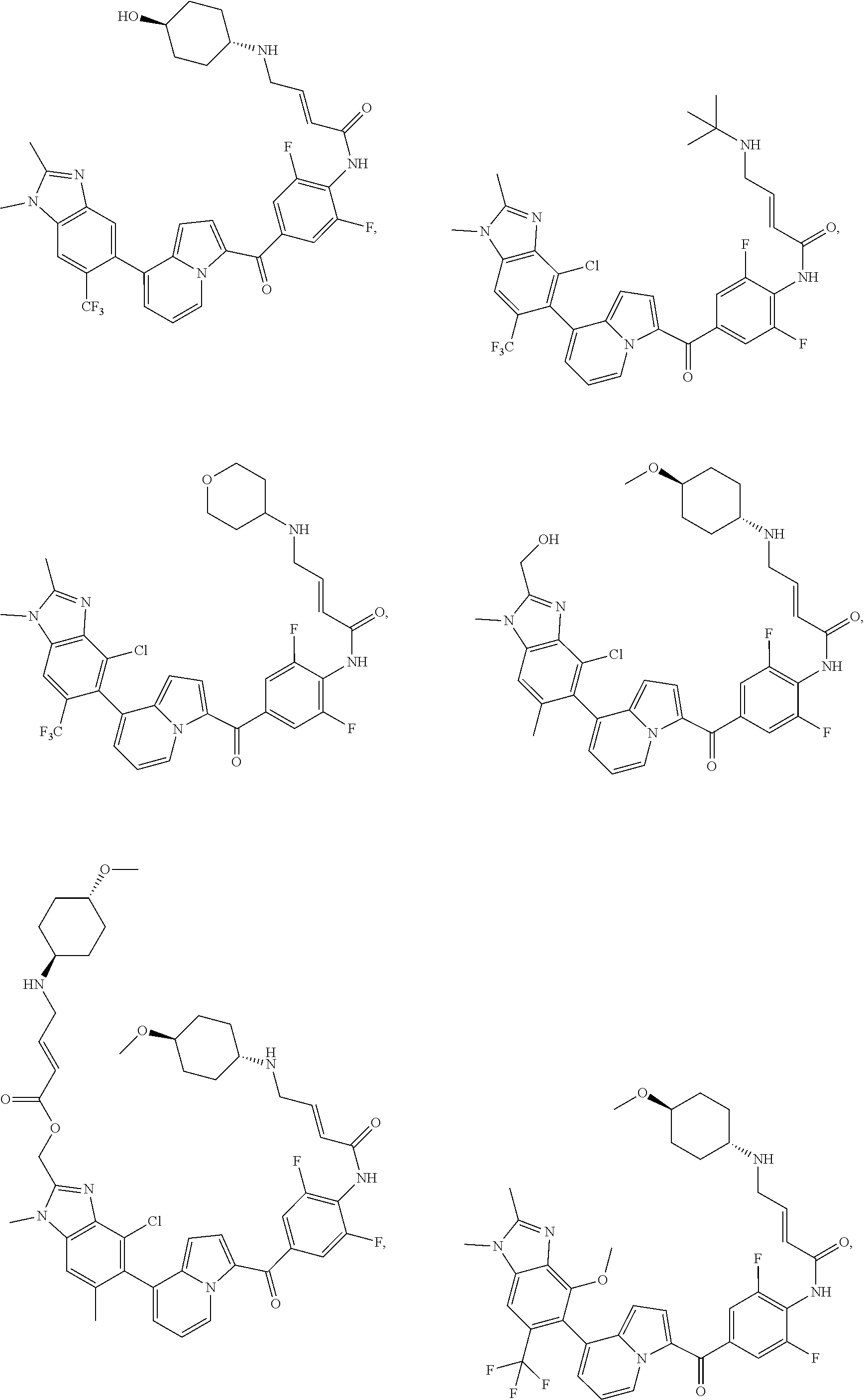 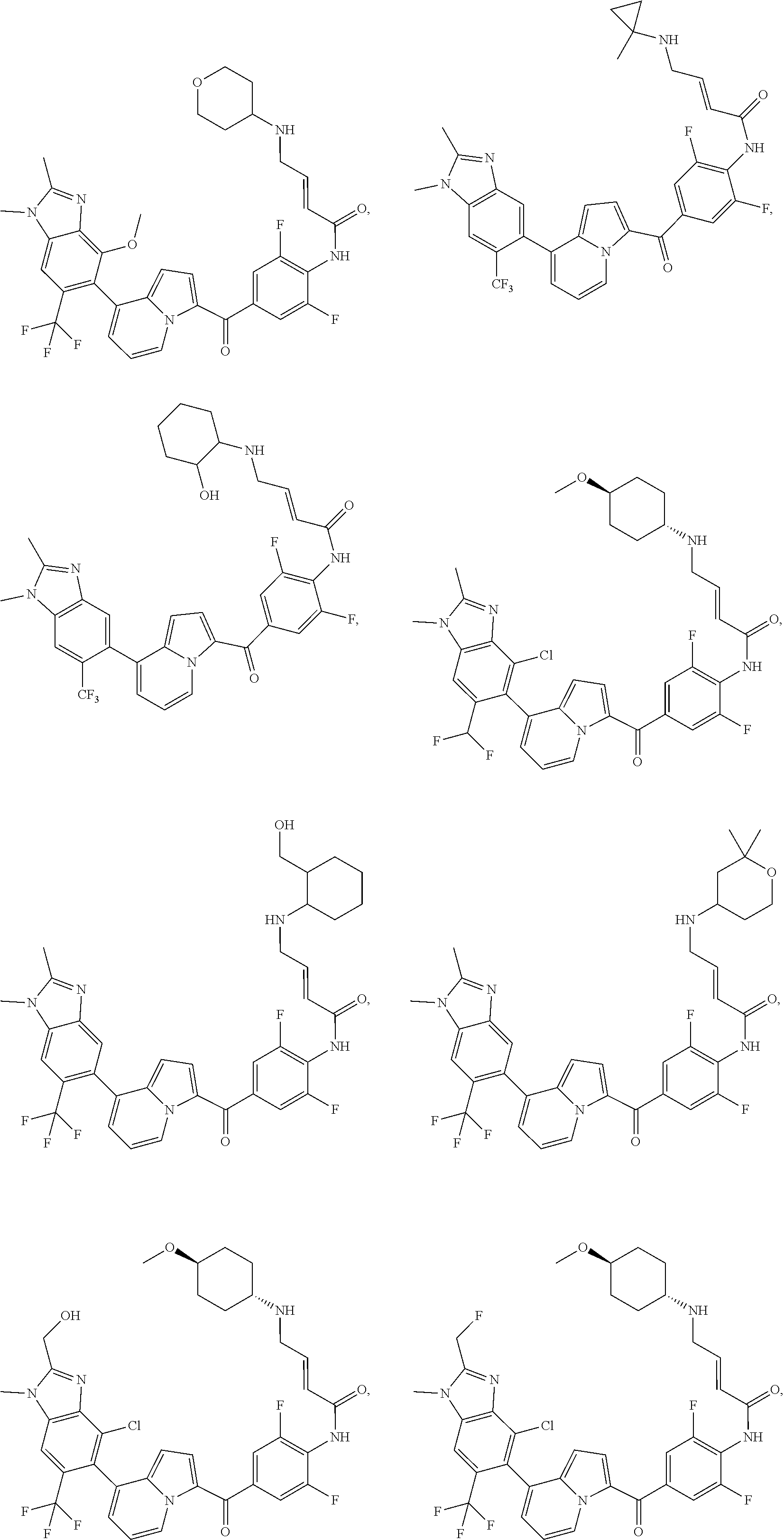 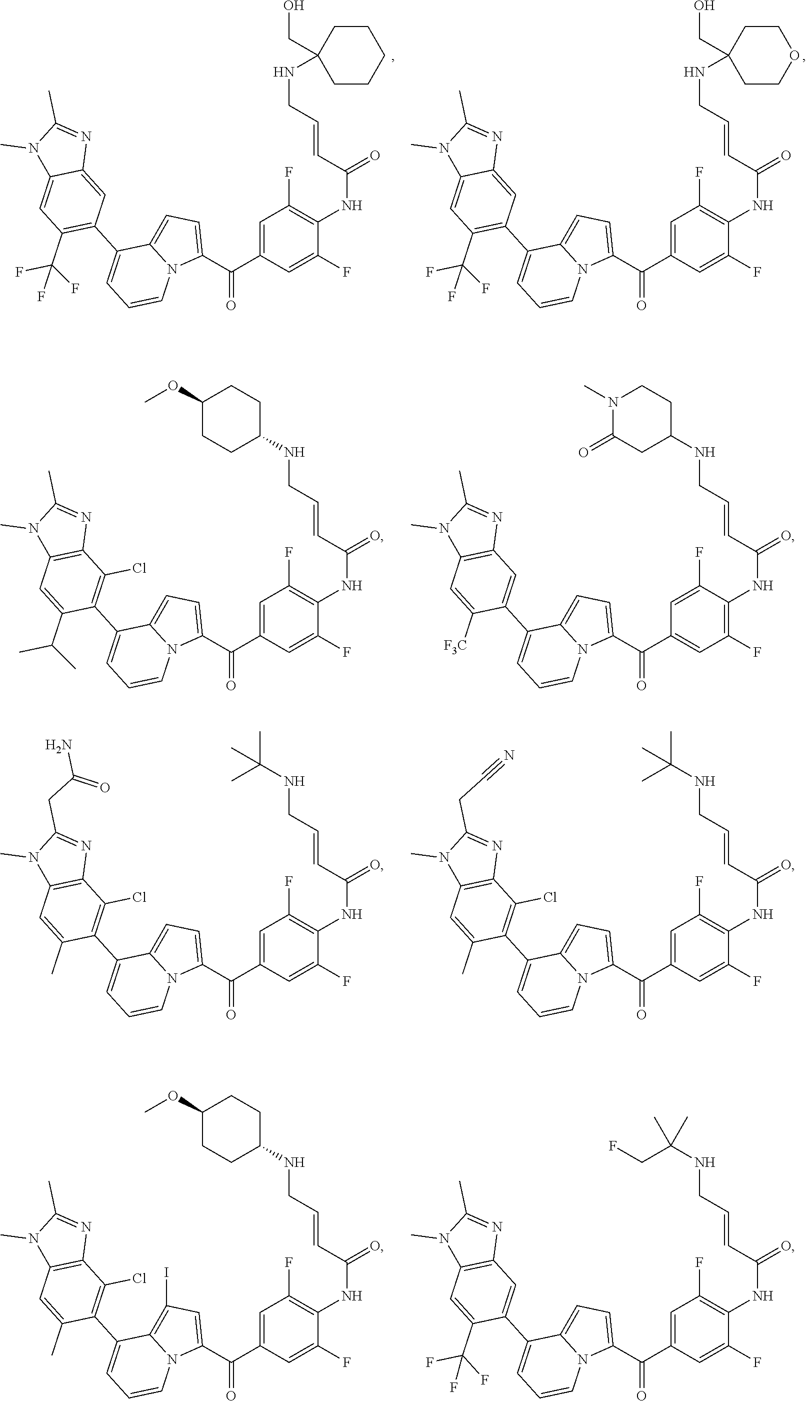 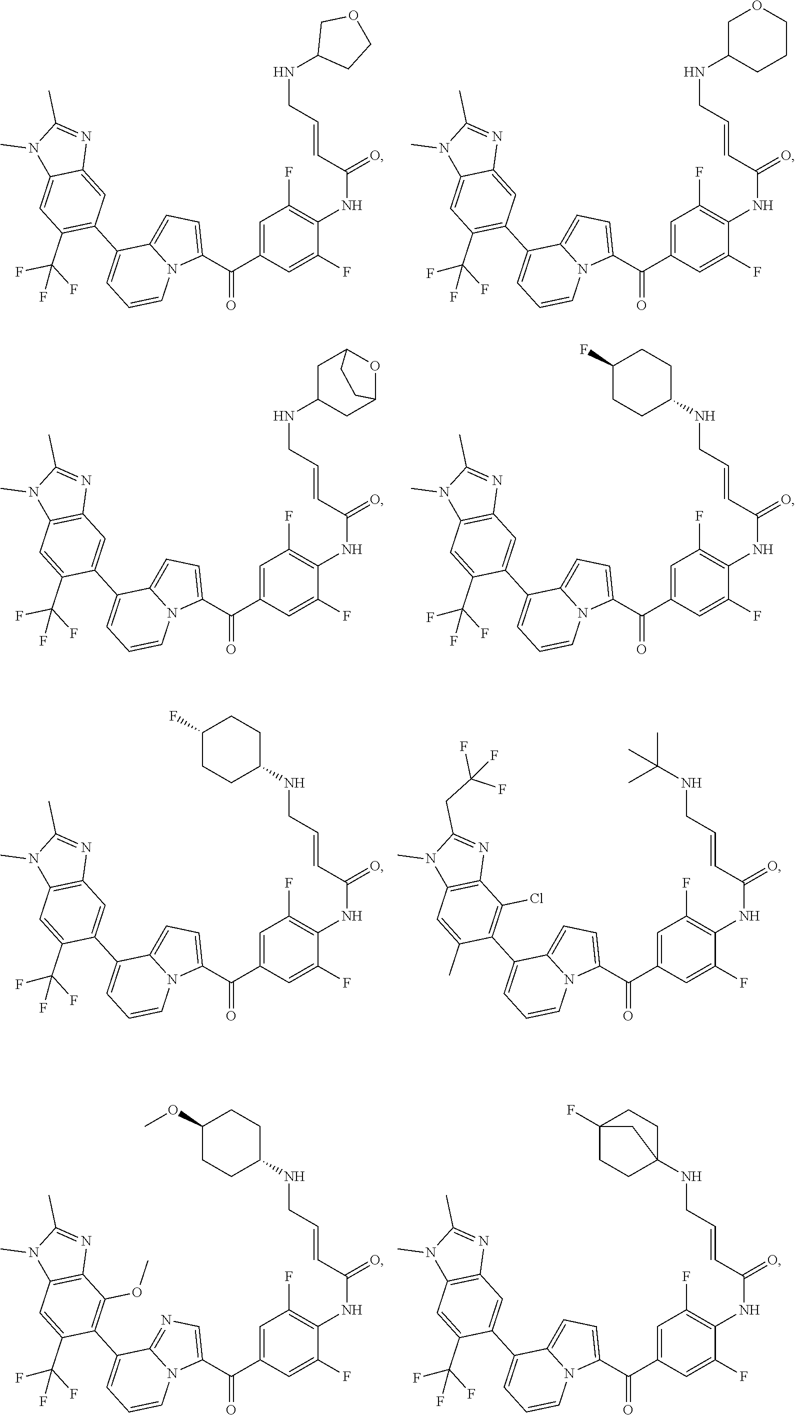 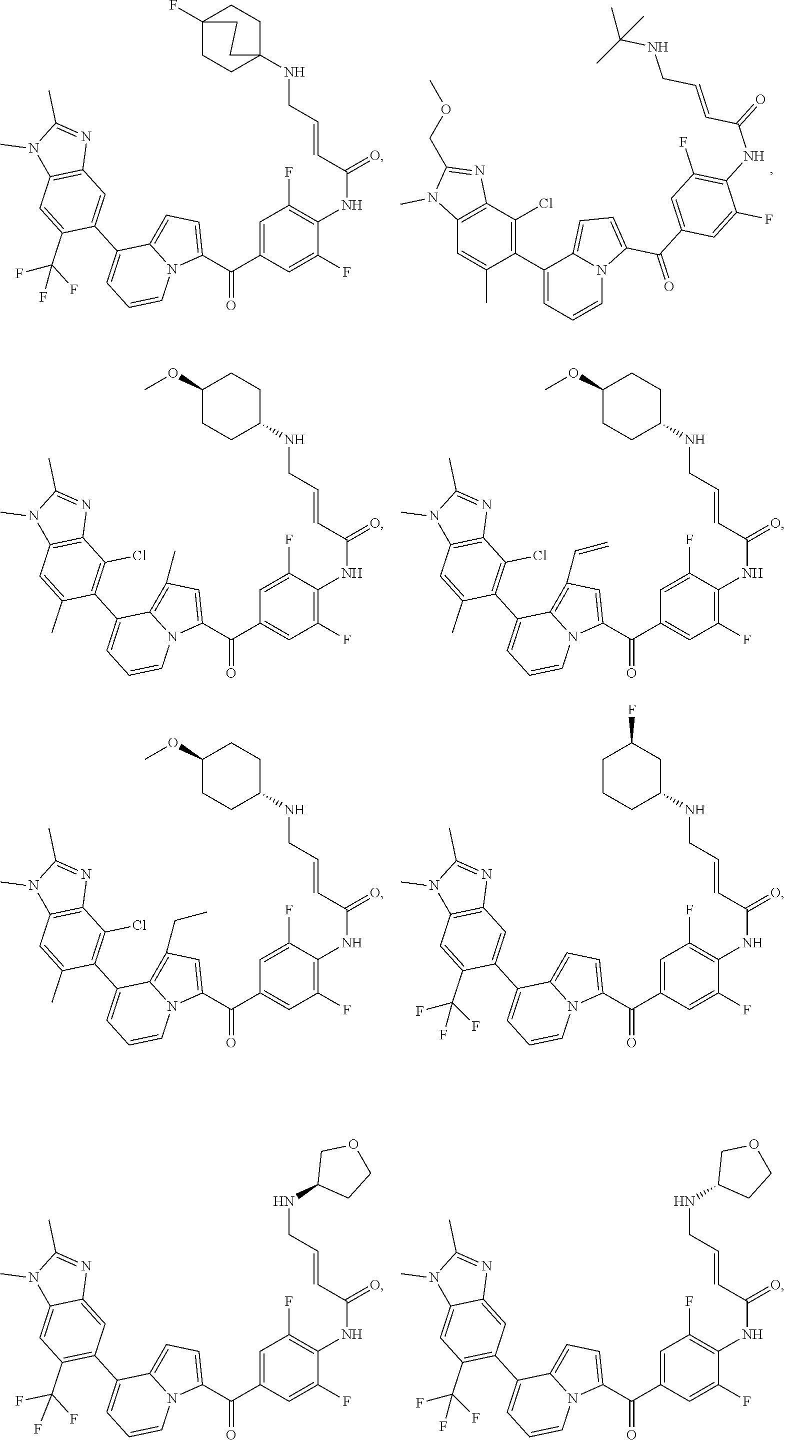 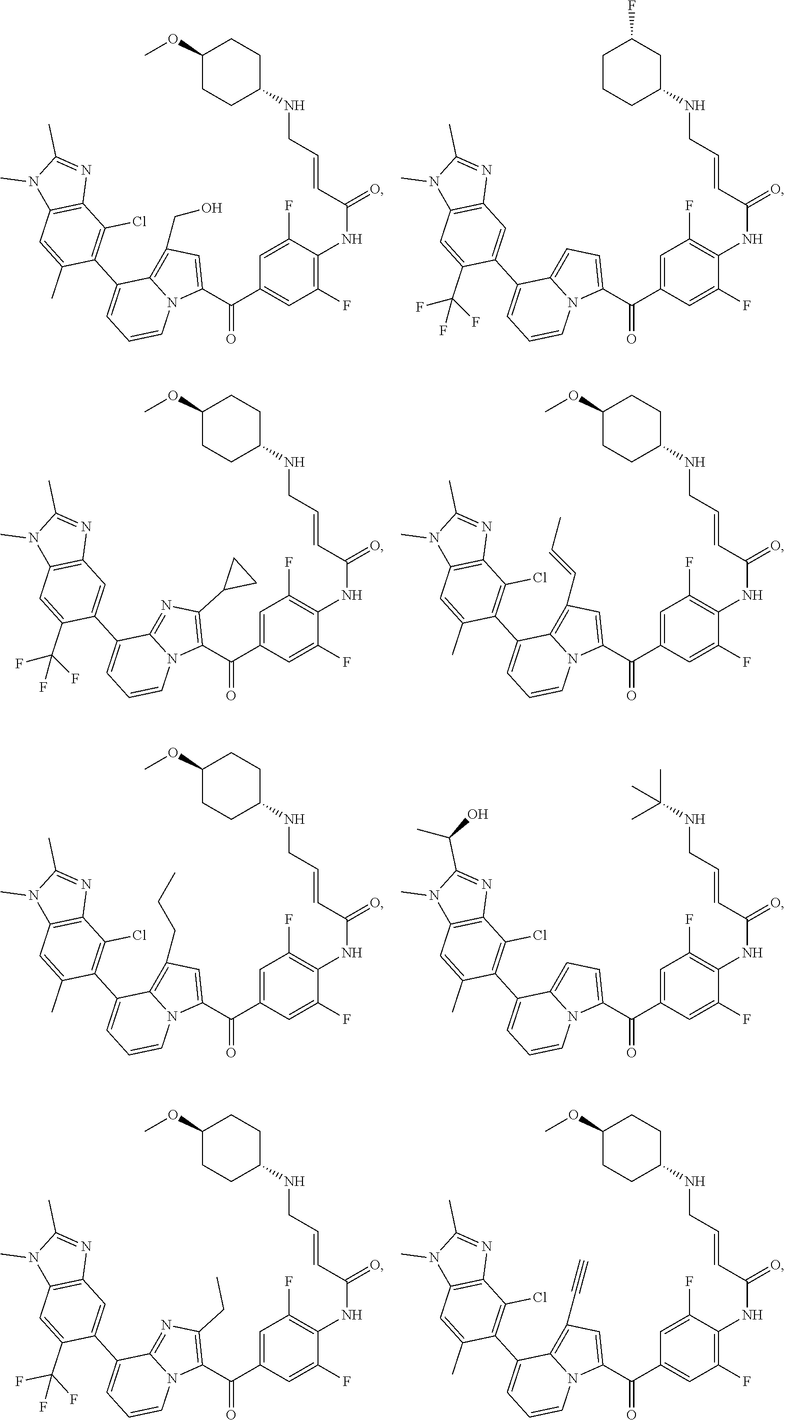 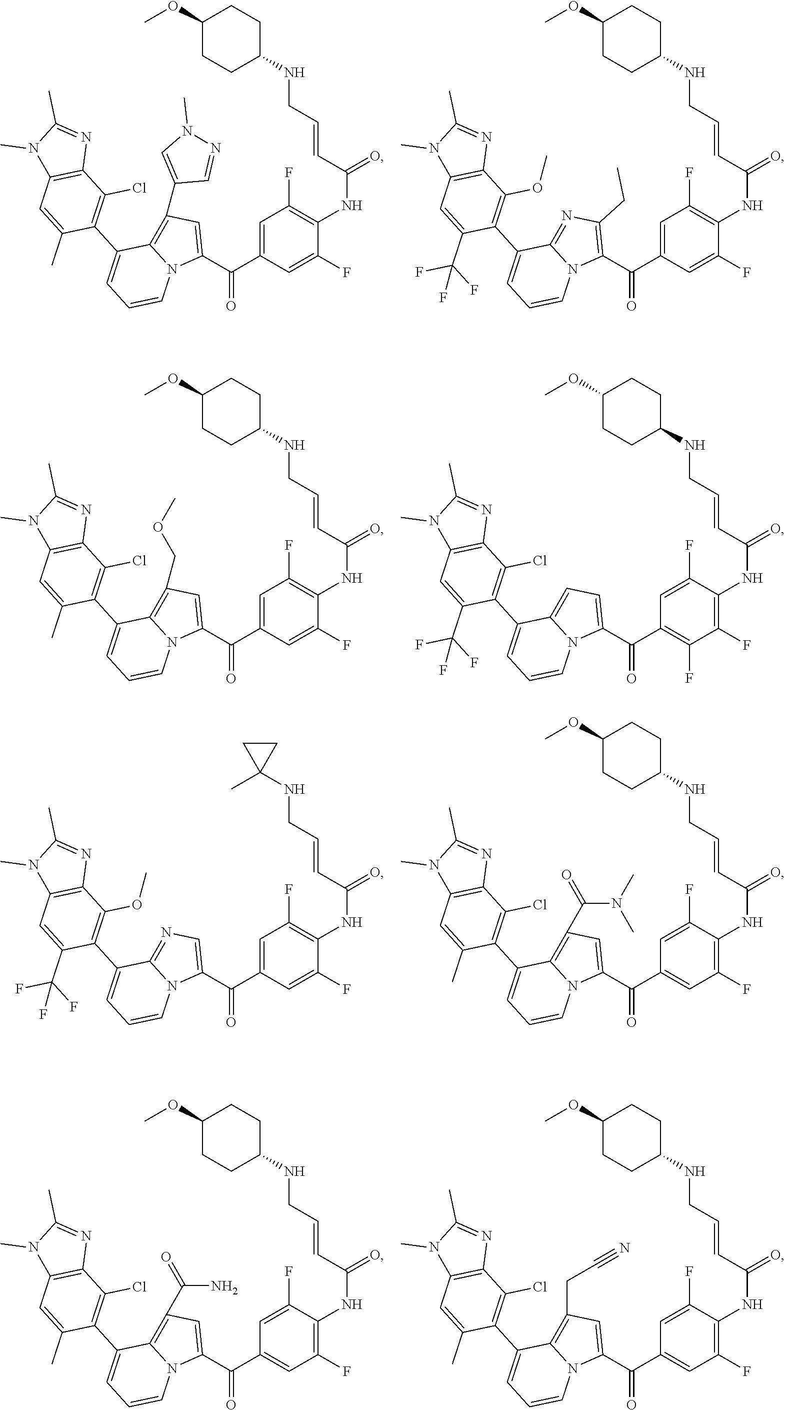 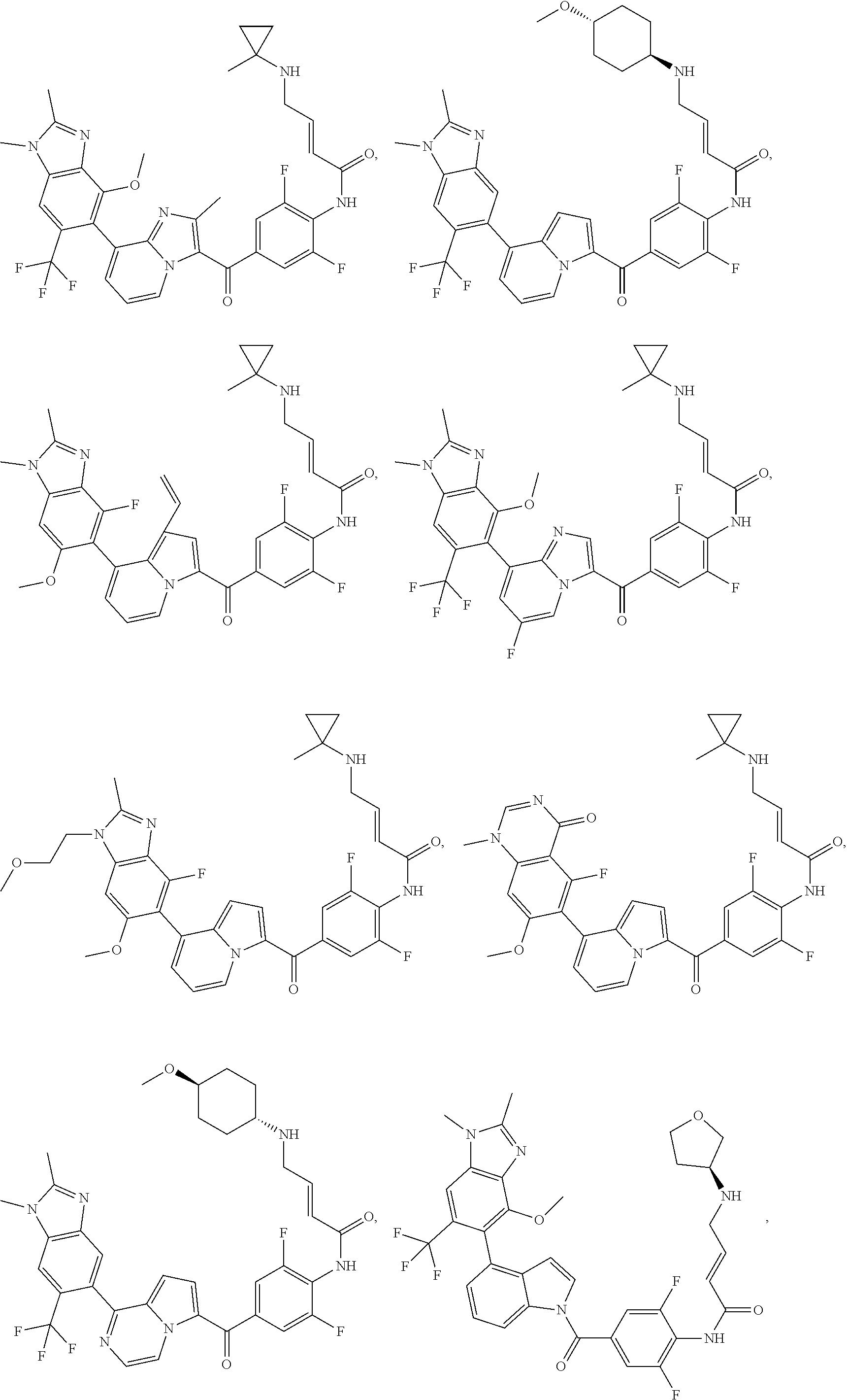 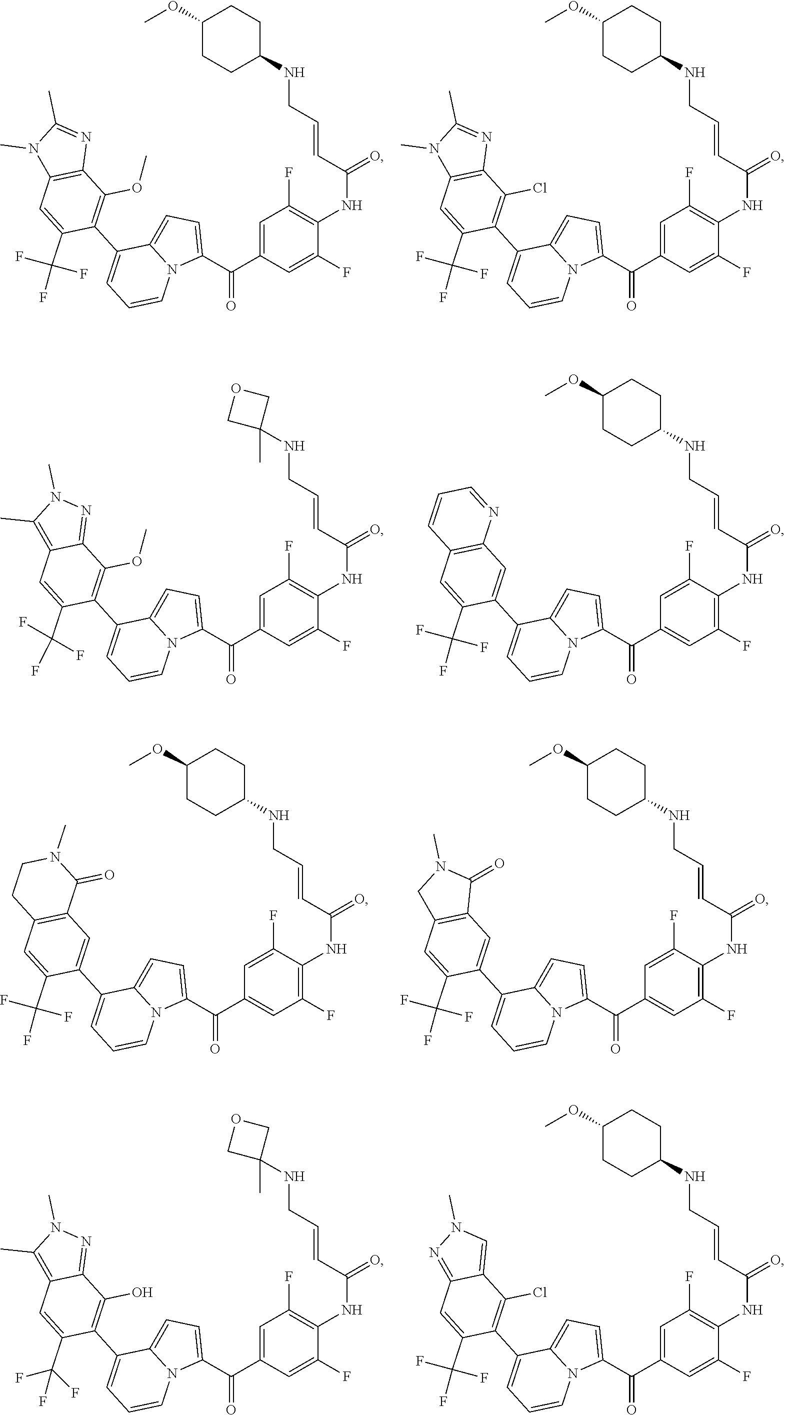 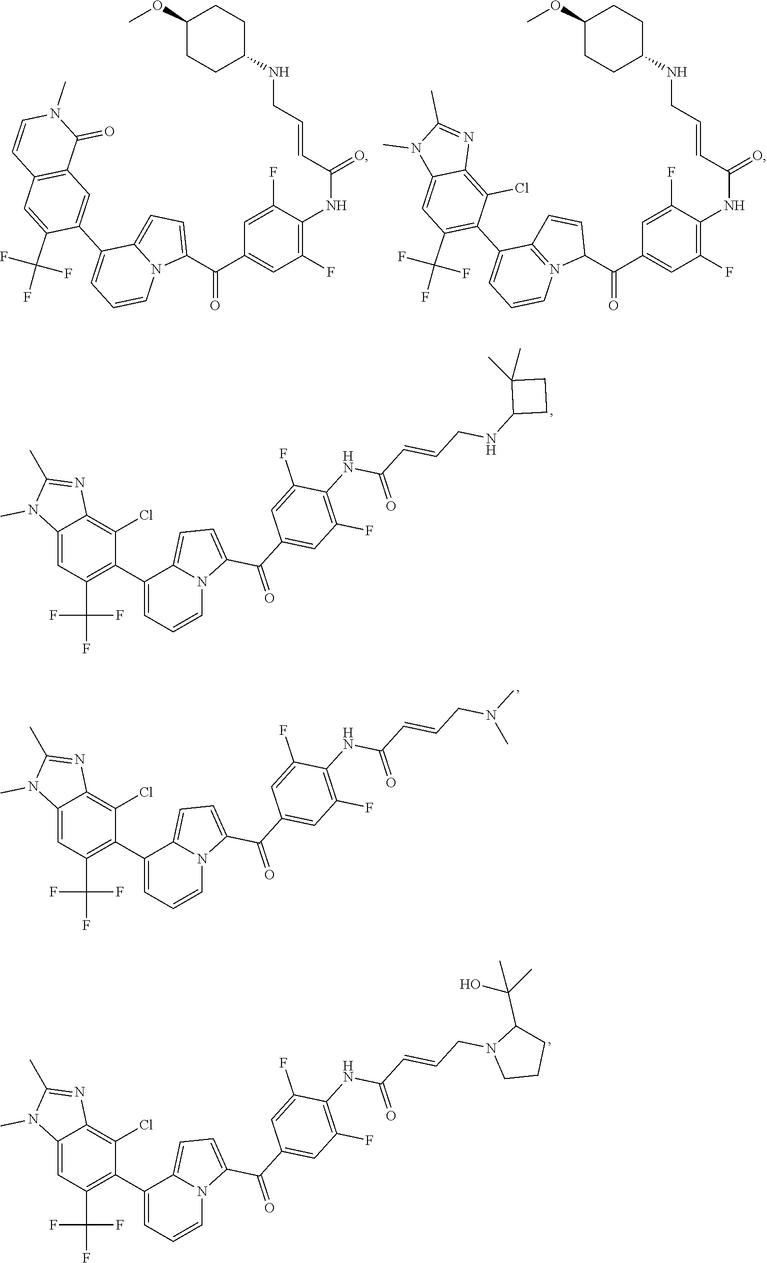 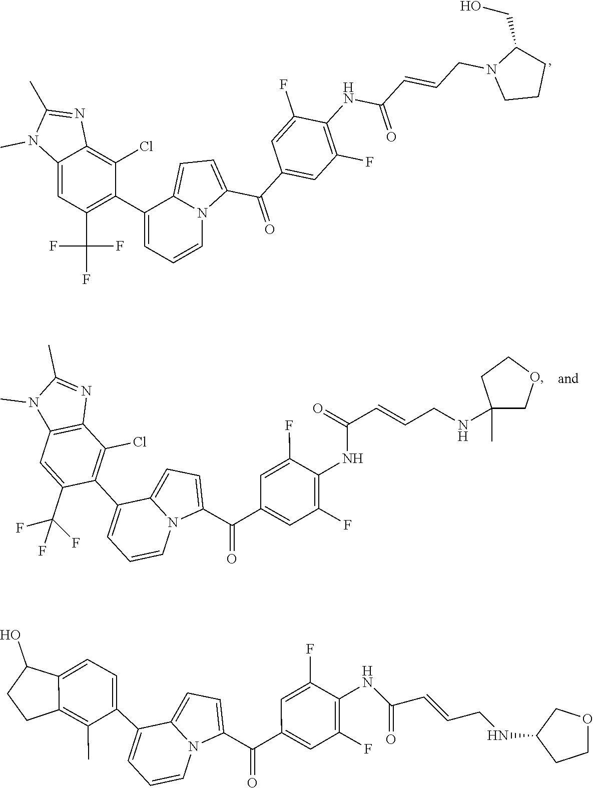 [SMILES: Cc1nc2c(Cl)c(-c3cccn4c(C(=O)c5cc(F)c(NC(=O)/C=C/CN6CCC[C@H]6CO)c(F)c5)ccc34)c(C(F)(F)F)cc2n1C ; Cc1nc2c(Cl)c(-c3cccn4c(C(=O)c5cc(F)c(NC(=O)/C=C/CNC6(C)CCOC6)c(F)c5)ccc34)c(C(F)(F)F)cc2n1C ; Cc1c(-c2cccn3c(C(=O)c4cc(F)c(NC(=O)/C=C/CN[C@H]5CCOC5)c(F)c4)ccc23)ccc2c1CCC2O]
or a pharmaceutically acceptable salt thereof.

2. A pharmaceutical composition comprising the compound of claim 1, or the pharmaceutically acceptable salt thereof, and a pharmaceutically acceptable carrier.

3. A pharmaceutical composition comprising the compound of claim 1 or the pharmaceutically acceptable salt thereof, an additional anti-cancer agent, and a pharmaceutically acceptable carrier.

4. A method of inhibiting KRAS G12C protein comprising contacting KRAS G12C protein with a compound of claim 1, or the pharmaceutically acceptable salt thereof, to inhibit the activity of the KRAS G12C protein.

5. A method of treating cancer comprising administering a therapeutically effective amount of a compound of claim 1, or the pharmaceutically acceptable salt thereof, to a subject in need of such treatment.

6. The method of claim 5, further comprising administering an additional active agent to the subject.

7.-13. (canceled)


SEQUENCE LISTING (3 sequences)

SEQ ID NO: 1 - protein, 170 aa, Artificial
  Synthetic Protein
GMTEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 2 - protein, 188 aa, Artificial
  Synthetic Protein
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 183 aa, Artificial
  Synthetic Protein
GLNDIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK